SEQUENCE LISTING (13 sequences)

SEQ ID NO: 1 - protein, 188 aa, Homo sapiens [sequence omitted]

SEQ ID NO: 2 - protein, 188 aa, Homo sapiens [sequence omitted]

SEQ ID NO: 3 - protein, 189 aa, Homo sapiens
MTEYKLVVVGASGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 4 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 5 - protein, 189 aa, Homo sapiens
MTEYKLVVVGASGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 7 - protein, 188 aa, Homo sapiens [sequence omitted]

SEQ ID NO: 8 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 9 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSED

SEQ ID NO: 10 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDD

SEQ ID NO: 11 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDD

SEQ ID NO: 12 - protein, 119 aa, Homo sapiens
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDEN

SEQ ID NO: 13 - protein, 120 aa, Homo sapiens
MAANKSKGQSSLALHKVIMVGSGGVGKSALTLQFMYDEFVEDYEPTKADSYRKKVVLDGEEVQIDILDTAGQEDYAAIRDNYFRSGEGFLLVFSITEHESFTATAEFREQILRVKAEEDK

(19) United States
(12) Patent Application Publication
(10) Pub. No.: US 2024/0116951 A1
(43) Pub. Date: Apr. 11, 2024

(54) HETEROCYCLIC COMPOUNDS AND USES THEREOF

(71) Applicant: Kumquat Biosciences Inc., San Diego, CA (US)

(72) Inventors: Xiaoming Li, San Diego, CA (US); Yuan Liu, San Diego, CA (US); Pingda Ren, San Diego, CA (US); Liansheng Li, San Diego, CA (US)

(21) Appl. No.: 18/311,850

(22) Filed: May 3, 2023

Related U.S. Application Data
(60) Provisional application No. 63/338,387, filed on May 4, 2022.
(60) Provisional application No. 63/491,723, filed on Mar. 22, 2023.

(51) Int. Cl. C07D 519/00; A61P 35/00; C07D 471/10; C07D 487/10

(52) U.S. Cl. CPC C07D 519/00; A61P 35/00; C07D 471/10; C07D 487/10

(57) ABSTRACT

The present disclosure provides compounds and pharmaceutically acceptable salts thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application claims the benefit of U.S. Provisional Application No. 63/338,387, filed May 4, 2022; and U.S. Provisional Application No. 63/491,723, filed Mar. 22, 2023, each incorporated herein by reference in its entirety.

### SEQUENCE LISTING

The instant application contains a Sequence Listing which has been submitted electronically in XML format and is hereby incorporated by reference in its entirety. Said XML copy, created on May 2, 2023, is named 56690_747_201_SL.xml and is 13,993 bytes in size.

### BACKGROUND

Cancer (e.g., tumor, neoplasm, metastases) is the second leading cause of death worldwide estimated to be responsible for about 10 million deaths each year. Many types of cancers are marked with mutations in one or more proteins involved in various signaling pathways leading to unregulated growth of cancerous cells. In some cases, about 25 to 30 percent (%) of tumors are known to harbor Rat sarcoma (Ras) mutations. In particular, mutations in the Kirsten Ras oncogene (K-Ras) gene are one of the most frequent Ras mutations detected in human cancers including lung adenocarcinomas (LUADs) and pancreatic ductal adenocarcinoma (PDAC).

Ras proteins have long been considered “undruggable,” due to, in part, high affinity to their substrate guanosine-5′-triphosphate (GTP) and/or their smooth surfaces without any obvious targeting region. The specific G12C Ras gene mutation has been identified as a druggable target to which a number of G12C specific inhibitors have been developed. However, such therapeutics are still of limited application due to drug resistance or relatively short duration of efficacy. In addition, drugging other mutant Ras molecules—including glycine to aspartate, glycine to valine, and glycine to serine at amino acid residue 12 or 13—remains difficult.

### SUMMARY

In view of the foregoing, there remains a considerable need for a new design of therapeutics and diagnostics that can specifically target Ras, including wildtype Ras, mutants and/or associated proteins of Ras to reduce Ras signaling output. Of particular interest are inhibitors, including pan Ras inhibitors capable of inhibiting two or more Ras mutants and/or wildtype Ras, as well as mutant-selective inhibitors targeting mutant Ras proteins such as Ras G12D, G12C, G12S, G13D, and/or G12V, for the treatment of Ras-associated diseases (e.g., cancer). Such compositions and methods can be particularly useful for treating a variety of diseases including, but not limited to, cancers and neoplasia conditions. The present disclosure addresses these needs, and provides additional advantages applicable for diagnosis, prognosis, and/or treatment for a wide diversity of diseases.

In an aspect is provided a compound of Formula (I), or a pharmaceutically acceptable salt or solvate thereof:

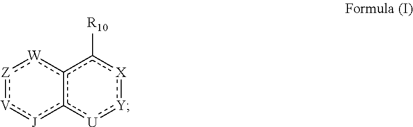

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), or S(O)₂;
U is N, C(R^{2c}), C(R^{2c})(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{2a};
R⁷ is

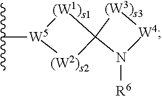

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20a};
R⁶ is -L²-R⁵ and wherein R⁶ is optionally capable of forming a covalent bond with a Ras amino acid;
L² is a bond, —O—, —N(R^{4d})—, —C(O)—, —S—, —S(O)₂—, —S(O)—, —P(O)R^{4d}—, —CR^{4c}R^{4c}, —OCR^{4c}R^{4c}—, —N(R^{4d})CR^{4c}R^{4c}—, —C(O)CR^{4c}R^{4c}—, —SCR^{4c}R^{4c}—, —S(O)₂CR^{4c}R^{4c}—, —S(O)CR^{4c}R^{4c}—, —P(O)R^{4d}CR^{4c}R^{4c}—, —CR^{4c}R^{4c}CR^{4c}R^{4c}, —CR^{4c}R^{4c}O—, —CR^{4c}R^{4c}N(R^{4d})—, —CR^{4c}R^{4c}C(O)—, —CR^{4c}R^{4c}S—, —CR^{4c}R^{4c}S(O)₂—, —CR^{4c}R^{4c}S(O)—, —CR^{4c}R^{4c}P(O)R^{4d}—, —N(R^{4d})C(O)—, —N(R^{4d})S(O)₂—, —N(R^{4d})S(O)—, —N(R^{4d})P(O)R^{4d}—, —C(O)N(R^{4d})—, —S(O)₂N(R^{4d})—, —S(O)N(R^{4d})—, —P(O)R^{4d}N(R^{4d})—, —OC(O)—, —OS(O)₂—, —OS(O)—, —OP(O)R^{4d}—, —C(O)O—, —S(O)₂O—, —S(O)O—, —P(O)R^{4d}O—, —CR^{4c}R^{4c}CR^{4c}CR^{4c}R^{4c}, —OCR^{4c}R^{4c}R^{4c}R^{4c}—, —N(R^{4d})CR^{4c}R^{4c}R^{4c}R^{4c}—, —C(O)CR^{4c}R^{4c}CR^{4c}R^{4c}—, —SCR^{4c}R^{4c}R^{4c}R^{4c}—, —S(O)₂CR^{4c}R^{4c}R^{4c}R^{4c}—, —S(O)CR^{4c}R^{4c}R^{4c}R^{4c}—, —P(O)R^{4c}R^{4c}R^{4c}R^{4c}R^{4c}—, —CR^{4c}R^{4c}R^{4c}R^{4c}O—, —CR^{4c}R^{4c}N(R^{4d})—, —CR^{4c}R^{4c}R^{4c}R^{4c}C(O)—, —CR^{4c}R^{4c}CR^{4c}R^{4c}S—, —CR^{4c}R^{4c}R^{4c}R^{4c}S(O)₂—, —CR^{4c}R^{4c}R^{4c}R^{4c}S(O)—, or —CR^{4c}R^{4c}R^{4c}R^{4c}P(O)R^{4d}—;
each R^{4c} is independently selected from hydrogen, halogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, —OC(O)R^{14a}, —N(R¹⁴)(R¹⁴), —N(R¹⁴)C(O)N(R¹⁴)(R¹⁴), —N(R¹⁴)C(O)OR¹⁴, —N(R¹⁴)C(O)R^{14a}, and —N(R¹⁴)S(O)₂R¹⁴, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R^{14a}, —S(O)₂N(R¹⁴)(R¹⁴), and —OC(O)R^{14a};
each R^{4d} is independently selected from hydrogen, —CN, C₁₋₆alkyl, C₂₋₆alkenyl, C₂₋₆alkynyl, C₁₋₆haloalkyl, C₁₋₆alkoxy, C₁₋₆haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, and —OC(O)R^{14a}, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆ alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R¹⁷ is -L¹-R¹⁹;
R^{17b} is -L^{1b}-R¹⁹;
   L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
   L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R²⁰ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═N)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R¹²)(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, —S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R² is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, —S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C_{2- 9}heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R^{11b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R^{20c}, R^{20d}, R^{20e}, R^{20f}, R^{20g}, R^{20h}, R^{20l}, R^{20k}, and R^{20m} are each independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉ heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.

In embodiments, R⁶ is capable of forming a covalent bond with a Ras amino acid sidechain. In embodiments, R⁶ is capable of forming a covalent bond with a KRas amino acid. In embodiments, R⁶ is capable of forming a covalent bond with the 12^{th} amino acid of a human KRas protein. In embodiments, R⁶ is capable of forming a covalent bond with the 12^{th} amino acid of a mutant KRas protein selected from KRas G12D, KRas G12C, and KRas G12S. In embodiments, R⁶ is capable of forming a covalent bond with the 13^{th} amino acid of a human KRas protein. In embodiments, R⁶ is capable of forming a covalent bond with the 13^{th} amino acid of a mutant KRas protein selected from KRas G13D, KRas G13C, and KRas G13S.

In embodiments, R⁶ is selected from the group consisting of

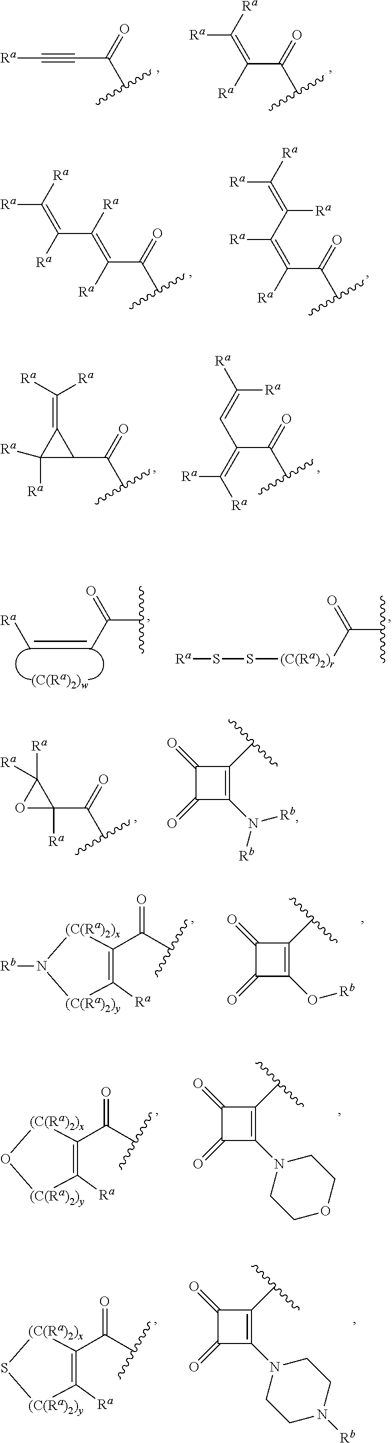

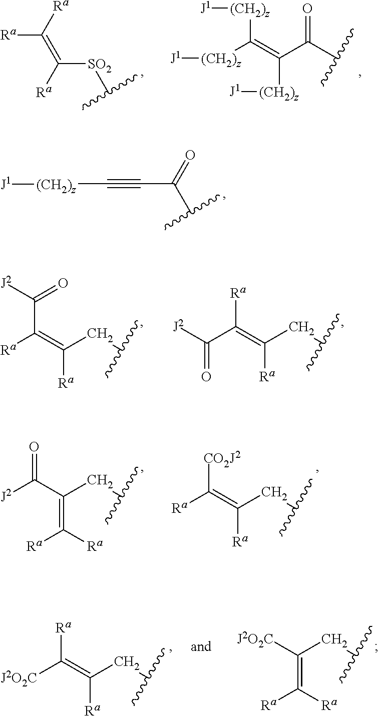

where each R^{a} is independently hydrogen, C₁₋₆ alkyl, carboxy, C₁₋₆carboalkoxy, phenyl, C₂₋₇carboalkyl, R^{1c}—(C(R^{b})₂)ᵣ—, R^{1c}—(C(R^{b})₂)_{w}-M-(C(R^{b})₂)ᵣ, (R^{d})(R^{e})CH-M- (C(R^{b})₂)ᵣ, or Het-J³-(C(R_{b})₂)ᵣ; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆ dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; each R^{1c} is independently —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; each M is independently —N(R^{b})—, —O—, —N[(C(R^{b})₂)_{w}NR^{b}R^{b}]—, or —N[(C(R^{b})₂)_{w}OR^{b}]—; each J³ is independently —N(R^{b})—, —O—, or a bond; each Het is independently a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; each r is independently 1-4; each w is independently 2-4; x is 0-1; y is 0-4, and each z is independently 1-6; wherein the sum of x+y is 2-4.

In embodiments, R⁶ is selected from the group consisting of

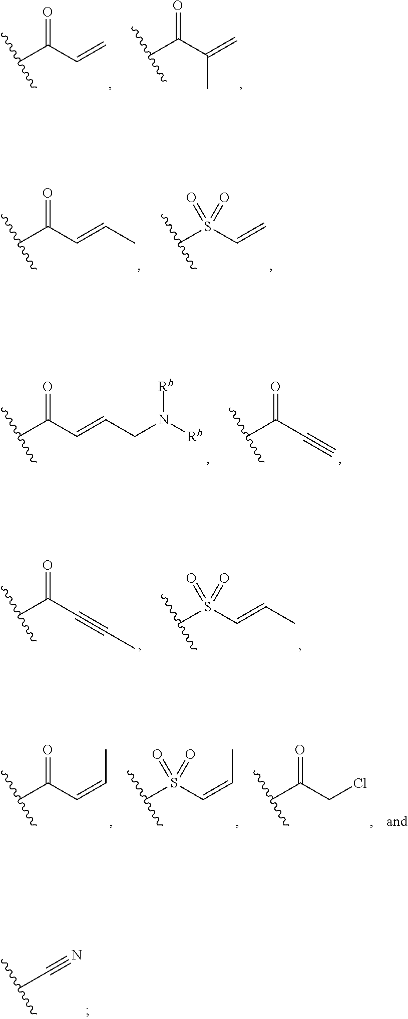

where each R^{b} is independently selected from the group consisting of hydrogen, hydroxyl, C₁-C₆ alkoxy, and C₁-C₆ alkyl.

In embodiments, L² is a bond, —C(O)NH—, —NHC(O)—, or —C(O)—; and
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k}.

In an aspect is provided a compound of Formula (II), or a pharmaceutically acceptable salt or solvate thereof:

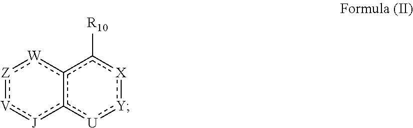

wherein
W is a N, C(R¹⁸), N(R^{18b}), C(R¹⁸)(R^{18a}), C(O), S(O), or S(O)₂;
Z is N, C(R¹⁸), N(R^{18b}), C(R¹⁸)(R^{18a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), S(O)₂, or C(O);
U is N, C(R²), C(R^{2c})(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is

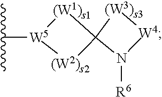

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20a};
R⁶ is -L²-R⁵ and wherein R⁶ is optionally capable of forming a covalent bond with a Ras amino acid;
L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R¹⁷ is -L¹-R¹⁹;
R^{17b} is -L^{1b}-R¹⁹;
   L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R¹), S(O)₂N(R^{1c}), S(O)N(R^{1c}), C(R^{1f})(R^{1g})O, C(R^{1f})(R^{1a})N(R^{1c}), and C(R)(R^{1g}); wherein the C₁-C₄alkyl, C₂-C₄alkenyl, and C₂-C₄alkynyl are optionally substituted with one, two, or three R²⁰ⁱ;
   L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, three, four, five, six, or seven R¹ⁱ
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═N)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R² is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C_{2- 9}heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R²;
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R²;
each R^{12c} is independently selected from hydrogen and R²;
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰;
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R^{18b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R^{20c}, R^{20d}, R^{20e}, R^{20f}, R^{20g}, R^{20h}, R²⁰ⁱ, R^{20k}, and R^{20m} are each independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉ heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.

In embodiments, R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹¹.

In embodiments, R⁶ is capable of forming a covalent bond with a Ras amino acid. In embodiments, R⁶ is capable of forming a covalent bond with a KRas amino acid. In embodiments, R⁶ is capable of forming a covalent bond with the 12^{th} amino acid of a human KRas protein. In embodiments, R⁶ is capable of forming a covalent bond with the 12^{th} amino acid of a mutant KRas protein selected from KRas G12D, KRas G12C, and KRas G12S. In embodiments, R⁶ is capable of forming a covalent bond with the 13^{th} amino acid of a human KRas protein. In embodiments, R⁶ is capable of forming a covalent bond with the 13^{th} amino acid of a mutant KRas protein selected from KRas G13D, KRas G13C, and KRas G13S.

In embodiments, R⁶ is selected from the group consisting of

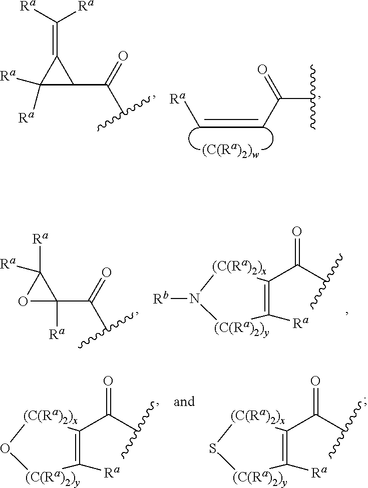

where each R^{a} is independently hydrogen, C₁₋₆ alkyl, carboxy, C₁₋₆carboalkoxy, phenyl, C₂₋₇carboalkyl, R^{1c}—(C(R^{b})₂)_{z}—, R^{e}—(C(R^{b})₂)_{w}-M-(C(R^{b})₂)ᵣ, (R^{d})(R^{e})CH-M- (C(R^{b})₂)ᵣ, or Het-J³-(C(R^{b})₂)ᵣ; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; each R^{c} is independently —NR^{b}R^{b} or —OR^{b}; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; each M is independently —N(R^{b})—, —O—, —N[(C(R^{b})₂)_{w}NR^{b}R^{b}]—, or —N[(C(R^{b})₂)_{w}OR^{b}]—; each J³ is independently —N(R^{b})—, —O—, or a bond; each Het is independently a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; each r is independently 1-4; each w is independently 2-4; x is 0-1; y is 0-4, and each z is independently 1-6; wherein the sum of x+y is 2-4.

In embodiments, R⁵ is a 5 or 6 membered partially unsaturated heterocycloalkyl or a 5 or 6 membered heteroaryl optionally substituted with one, two or three R^{20k}, wherein the partially unsaturated 5 or 6 membered heterocycloalkyl or 5 or 6 membered heteroaryl comprises one, two, or three ring nitrogen atoms; and is bonded to L² through a ring nitrogen.

In embodiments, R⁶ is

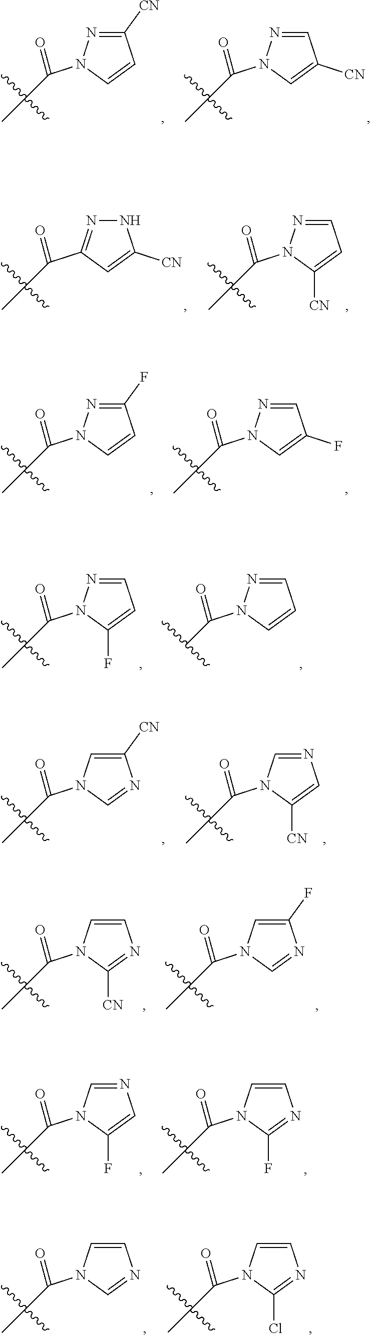

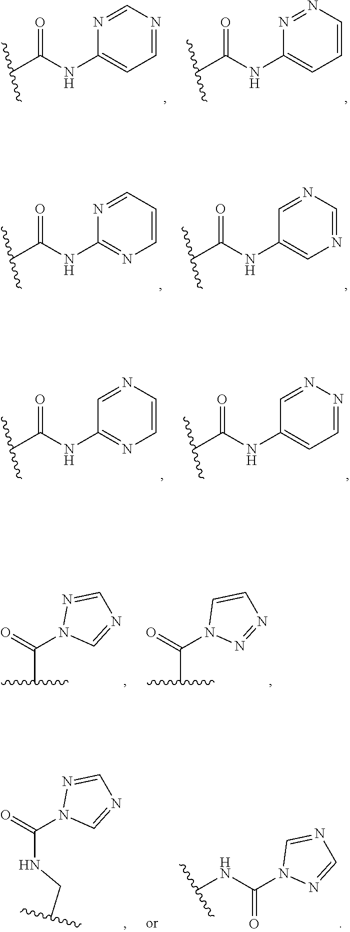

In embodiments, R⁶ is not capable of forming a covalent bond with the 12^{th} amino acid of a human KRas protein selected from KRas wildtype, KRas G12D, KRas G12C, KRas G12S, KRas G12V, KRas G13D, KRas G13C, KRas G13S, and KRas G13V. In embodiments, R⁶ is not capable of forming a covalent bond with the 13^{th} amino acid of human KRas protein selected from KRas wildtype, KRas G12D, KRas G12C, KRas G12S, KRas G12V, KRas G13D, KRas G13C, KRas G13S, and KRas G13V. In embodiments, R⁶ is not capable of forming a covalent bond with a KRas amino acid. In embodiments, R⁶ is not capable of forming a covalent bond with a Ras amino acid.

In embodiments, L² is a bond, —C(O)NH—, —NHC(O)—, or —C(O)—; and
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k}.

In embodiments, L² is —C(O)—; and R⁵ is a C₃₋₁₂cycloalkyl optionally substituted with one, two or three R^{2k}. In embodiments, L² is —C(O)—; and R⁵ is a cyclopropyl optionally substituted with one, two or three R^{20k} selected from halogen and CN.

In embodiments, R⁶ is

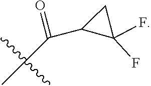 [SMILES: *C(=O)C1CC1(F)F]

In embodiments, R⁶ is

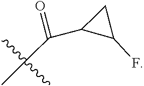 [SMILES: *C(=O)C1CC1F]

In embodiments, R⁶ is

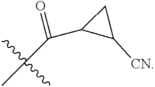 [SMILES: *C(=O)C1CC1C#N]

In embodiments, R⁶ is

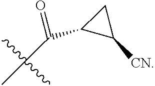 [SMILES: *C(=O)[C@@H]1C[C@H]1C#N]

In embodiments, L² is a bond, —C(O)NH—, —NHC(O)—, or —C(O)—;
L² is bonded to a carbon atom of R⁵; and
R⁵ is selected from —CN, C₁₋₆alkyl, C₂₋₆alkenyl, C₂₋₆alkynyl, C₃₋₁₂cycloalkyl, C₆₋₁₂aryl, and C₁₋₁₁heteroaryl, wherein C₁₋₆alkyl, C₂₋₆alkenyl, C₂₋₆alkynyl, C₃₋₁₂cycloalkyl, C₆₋₁₂aryl, and C₁₋₁₁heteroaryl, are optionally substituted with one, two, or three R^{20k};

In embodiments, L² is —C(O)—;
L² is bonded to a carbon atom of R⁵; and
R⁵ is selected from C₁₋₆alkyl, C₂₋₆alkenyl, C₂₋₆alkynyl, C₃₋₁₂cycloalkyl, C₆₋₁₂aryl, and C₁₋₁₁heteroaryl, wherein C₁₋₆alkyl, C₂₋₆alkenyl, C₂₋₆alkynyl, C₃₋₁₂cycloalkyl, C₆₋₁₂aryl, and C₁₋₁₁heteroaryl, are optionally substituted with one, two, or three R^{20k};

In embodiments, L² is —C(O)—;
L² is bonded to a carbon atom of R⁵; and
R⁵ is selected from C₁₋₆alkyl, C₂₋₆alkenyl, C₂₋₆alkynyl, C₃₋₁₀cycloalkyl, and 5-6 membered heteroaryl, wherein C₁₋₆alkyl, C₂₋₆alkenyl, C₂₋₆alkynyl, C₃₋₁₀cycloalkyl, and 5-6 membered heteroaryl are optionally substituted with one, two, or three R^{20k}.

In embodiments, L² is —C(O)—;
R⁵ is a heteroaryl having the formula:

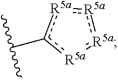

R^{5a} is independently O, S, CH, C(R^{20k}), N, NH, or N(R^{20k});
R⁵ comprises 0-3 independent R^{20k}; and
0-4 R^{5a} are independently N, NH, or N(R^{20k}.

In embodiments, L² is —C(O)—; and
R⁵ is a heteroaryl having the formula:

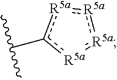

R^{5a} is independently CH, C(R^{20k}), N, NH, or N(R^{20k});
R⁵ comprises 0-3 independent R^{20k}; and
0-4 R^{5a} are independently N, NH, or N(R^{20k}.

In embodiments, L² is —C(O)—;
R⁵ is

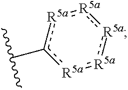

R^{5a} is independently CH, C(R^{20k}), CH(R^{20k}), CH₂, C(R^{20k})₂, N, NH, or N(R²⁰ⁱ);
R⁵ comprises 0-3 independent R^{20k}; and
0-4 R^{5a} are independently N, NH, or N(R^{20k}).

In embodiments, L² is —C(O)—; and R⁵ is selected from C₂₋₆alkenyl and C₂₋₆alkynyl, wherein C₂₋₆alkenyl and C₂₋₆alkynyl are optionally substituted with one, two, or three R^{20k}.

In an aspect is provided a compound of Formula (IV), or a pharmaceutically acceptable salt or solvate thereof:

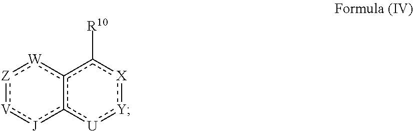

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16a}); wherein exactly one of V and J is C(R¹⁷), C(R¹⁷)(R^{16a}), or N(R^{17b});
Y is N, C(R²), C(R²)(R^{2c}), N(R^{2b}), S(O), S(O)₂, or C(O);
U is N, C(R²), C(R²)(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{2a};

R⁷ is

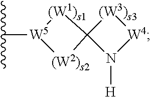

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6 and s2 is an integer from 2 to 3; or s1 is an integer from 2 to 6 and s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20a};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R^{8b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R¹⁷ is -L¹-R¹⁹;
R^{17b} is -L^{1b}-R¹⁹;
   L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R¹ⁱ)(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
   L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1a})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₀heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R^{16b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R² is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═Ni)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{2d};
each R^{12″} is independently selected from C₃₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₃₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R^{18b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
substituted with one, two, or three R^{20h};
each R^{20a}, R^{20b}, R^{20c}, R^{20d}, R^{20e}, R^{20f}, R^{20g}, R^{20h}, R²⁰ⁱ, and R^{20m} are each independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl; and
[symbol] indicates a single or double bond such that all valences are satisfied.

In embodiments, Y is C(O). In embodiments, Y is N. In embodiments, Y is C(R²). In embodiments, Y is C(R²)(R^{2c}). In embodiments, Y is S(O). In embodiments, Y is S(O)₂. In embodiments, X is N. In embodiments, X is C(R³). In embodiments, X is C(R³)(R³). In embodiments, X is N(R³). In embodiments, U is N. In embodiments, U is C(R^{2c}). In embodiments, U is C(R^{2c})(R^{2c}). In embodiments, U is N(R^{2b}). In embodiments, U is S(O). In embodiments, U is S(O)₂. In embodiments, U is C(O). In embodiments, W is a N. In embodiments, W is a C(R¹⁸). In embodiments, W is a N(R^{18b}). In embodiments, W is a C(R¹⁸)(R^{18a}). In embodiments, W is a C(O). In embodiments, W is a S(O). In embodiments, W is a S(O)₂. In embodiments, Z is N. In embodiments, Z is C(R⁸). In embodiments, Z is N(R^{16b}). In embodiments, Z is C(R¹⁶)(R^{16a}). In embodiments, Z is C(O). In embodiments, Z is S(O). In embodiments, Z is S(O)₂. In embodiments, V is N(R^{16b}). In embodiments, V is N. In embodiments, V is C(R¹⁶)(R^{16b}). In embodiments, V is C(R¹⁶). In embodiments, V is N(R^{17b}). In embodiments, V is C(R¹⁷)(R^{16a}). In embodiments, V is C(R¹⁷). In embodiments, J is N(R¹⁶). In embodiments, J is N. In embodiments, J is C(R¹⁶)(R^{16a}). In embodiments, J is C(R¹⁶). In embodiments, J is N(R^{17b}). In embodiments, J is C(R¹⁷)(R^{16a}). In embodiments, J is C(R¹⁷). In embodiments, L⁷ is a bond. In embodiments, W¹ and W³ are independently selected from NH, CH₂, C(O), S, O, S(O), and S(O)₂. In embodiments, W¹ and W³ are independently CH₂. In embodiments, W² is independently selected from a bond, NH, CH₂, C(O), S, O, S(O), and S(O)₂. In embodiments, W² is a bond. In embodiments, W² is CH₂. In embodiments, W⁴ is CH₂. In embodiments, W⁵ is N. In embodiments, W⁵ is CH. In embodiments, s1 is 1. In embodiments, s1 is 2. In embodiments, s1 is 3. In embodiments, s1 is 4. In embodiments, s1 is 5. In embodiments, s1 is 6. In embodiments, s2 is 1. In embodiments, s2 is 2. In embodiments, s2 is 3. In embodiments, s3 is 1. In embodiments, s3 is 2. In embodiments, s3 is 3. In embodiments, L¹ is a bond and Lib is a bond. In embodiments, R¹⁹ is selected from a bicyclic C₄₋₁₂cycloalkyl, bicyclic C₂₋₁₁heterocycloalkyl, bicyclic C₇₋₁₂aryl, and bicyclic C₂₋₁₂heteroaryl, wherein the C₄₋₁₂cycloalkyl, bicyclic C₂₋₁₁heterocycloalkyl, bicyclic C₇₋₁₂aryl, and bicyclic C₂₋₁₂heteroaryl are optionally substituted with one, two, three, four, five, six, or seven R¹ⁱ.

In embodiments, R¹⁹ is selected from abridged bicyclic C₄₋₁₂cycloalkyl, bridged bicyclic C₂₋₁₁heterocycloalkyl, bridged bicyclic C₇₋₁₂aryl, and bridged bicyclic C₂₋₁₂heteroaryl, wherein the bridged bicyclic C₄₋₁₂cycloalkyl, bridged bicyclic C₂₋₁₁heterocycloalkyl, bridged bicyclic C₇₋₁₂aryl, and bridged bicyclic C₂₋₁₂heteroaryl are optionally substituted with one, two, three, four, five, six, or seven R¹ⁱ.

In embodiments, R¹⁹ is selected from a fused bicyclic C₄₋₁₂cycloalkyl, fused bicyclic C₂₋₁₁heterocycloalkyl, fused bicyclic C₇₋₁₂aryl, and fused bicyclic C₂₋₁₂heteroaryl, wherein the fused bicyclic C₄₋₁₂cycloalkyl, fused bicyclic C₂₋₁₁heterocycloalkyl, fused bicyclic C₇₋₁₂aryl, and fused bicyclic C₂₋₁₂heteroaryl are optionally substituted with one, two, three, four, five, six, or seven R¹ⁱ.

In embodiments, R¹⁹ is

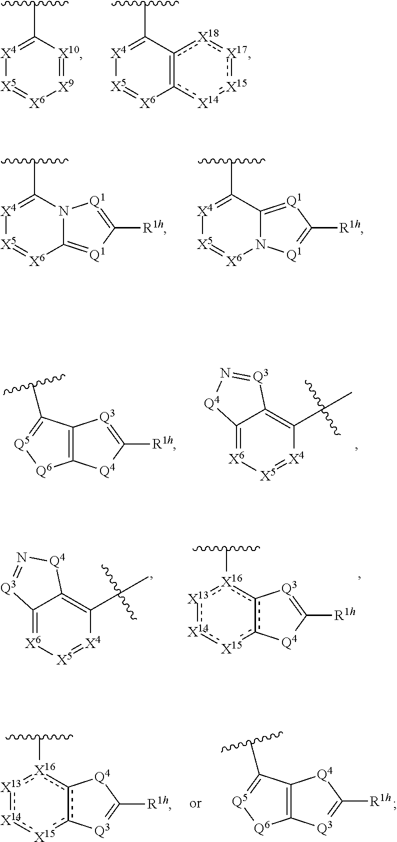

Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, X¹⁸ are independently selected from a C(O), C(R^{1a}), N, C(R^{1a})(R^{1b}), and N(R^{1c}); X¹⁶ are independently selected from C, N, and C(R^{1a});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments, R¹⁹ is selected from:

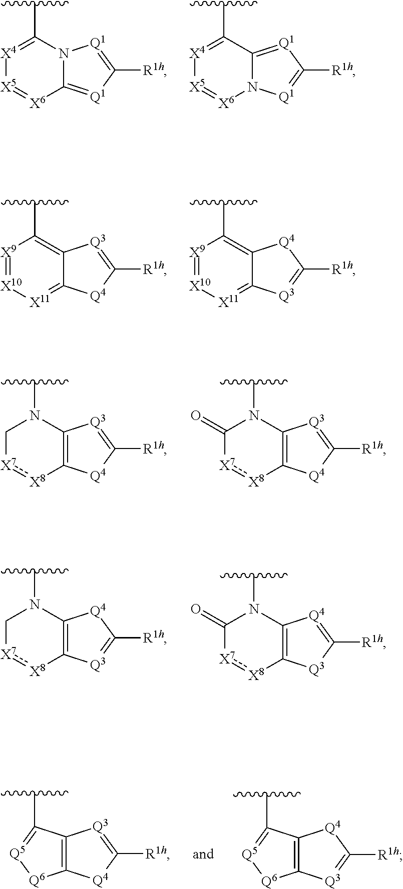

Q¹, Q³, and Q⁵ are independently N or C(R^{1d});
Q⁴ and Q⁶ are independently O, S, C(R^{1a})(R^{1b}), or N(R^{1c});
X⁴, X⁵, X⁶, X⁹, X¹⁰, and X¹¹ are independently selected from C(R^{1a}) and N;
X⁷ and X⁸ are independently selected from C(R^{1a}), C(R^{1a})(R^{1b}), N, and N(R^{1c});
each R^{1a}, R^{1b}, R^{1d}, and R^{1h} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments, R¹⁹ is selected from

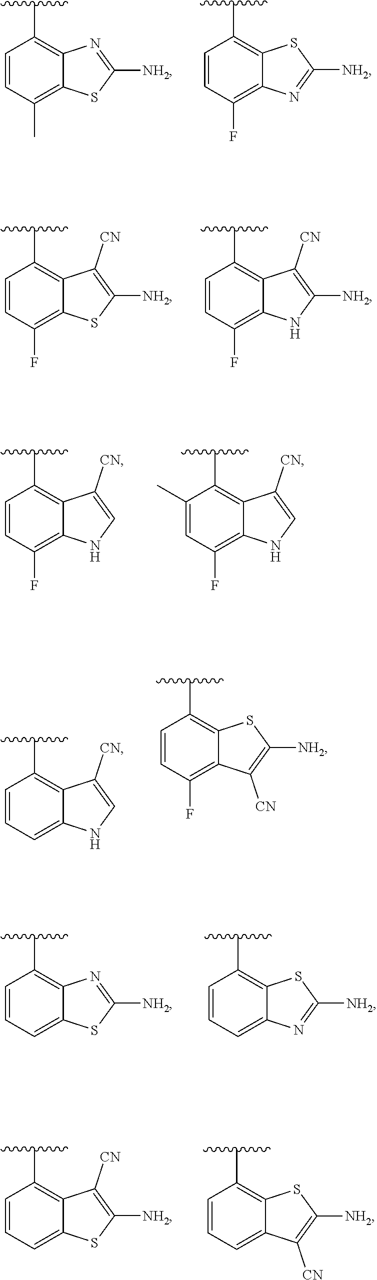

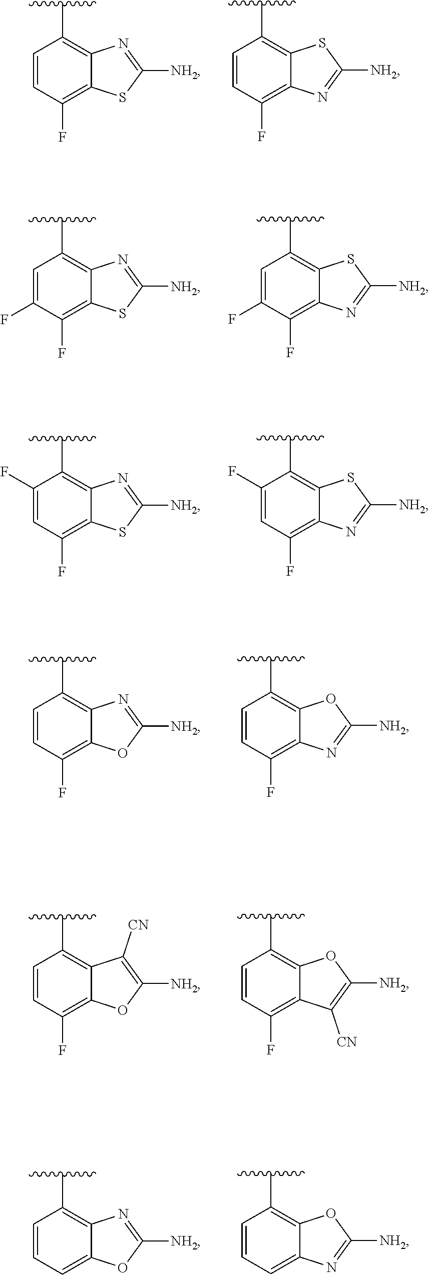

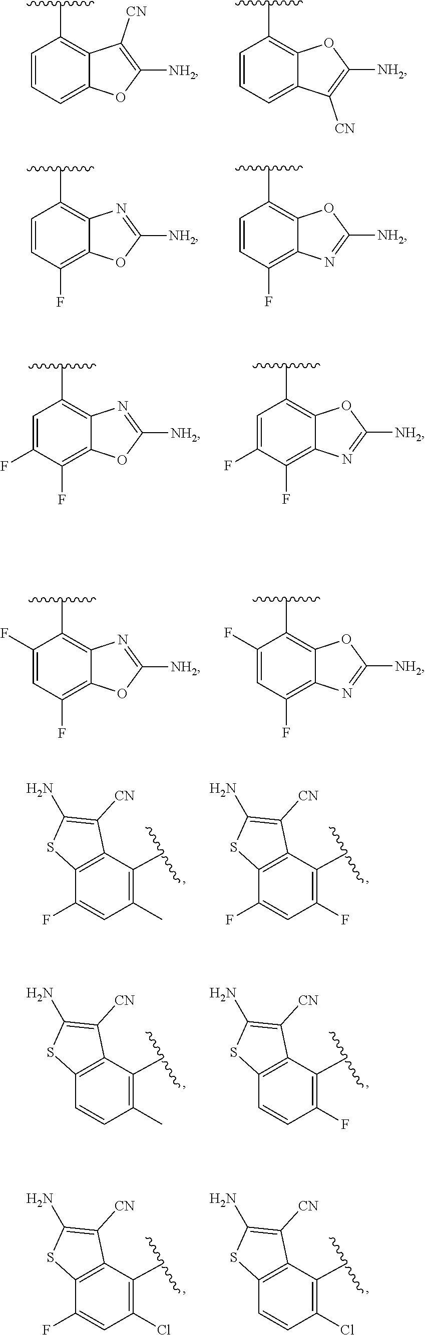

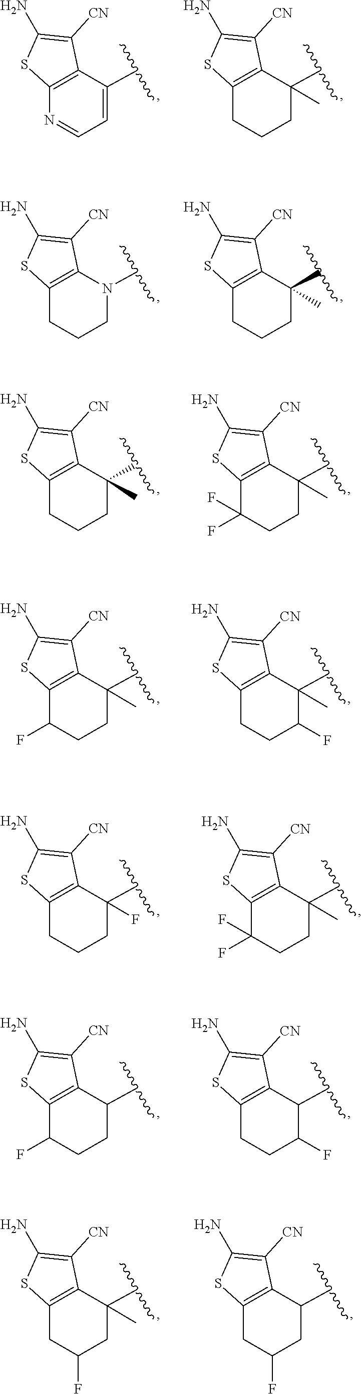

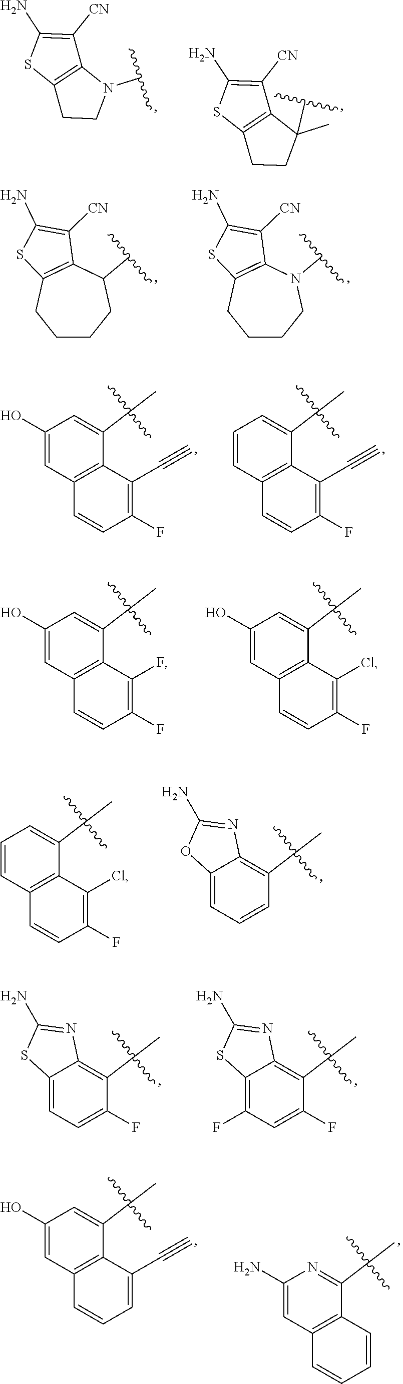

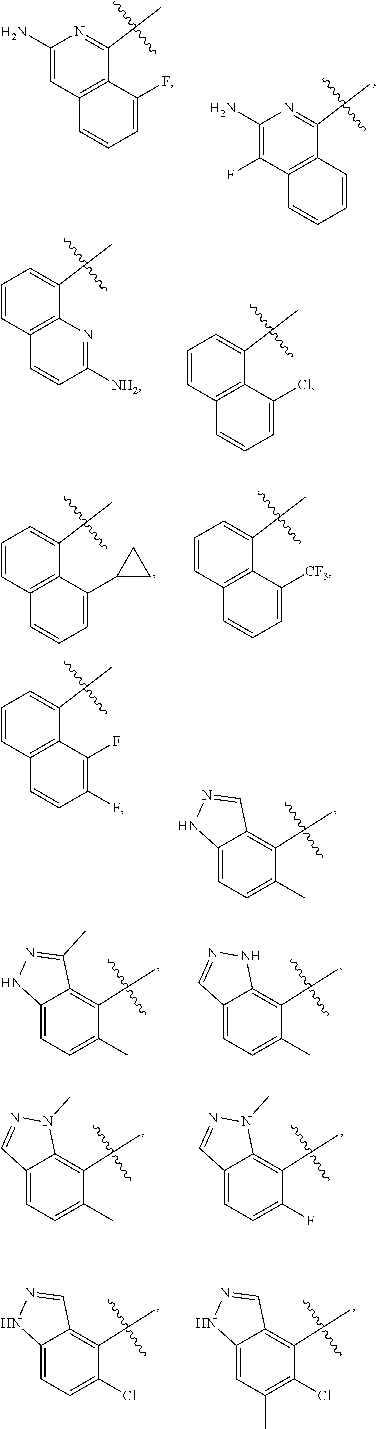

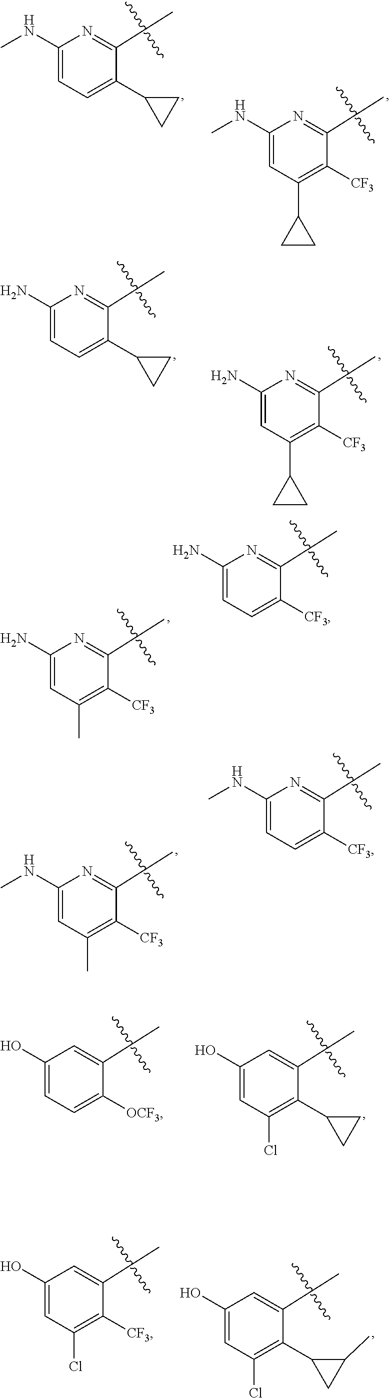

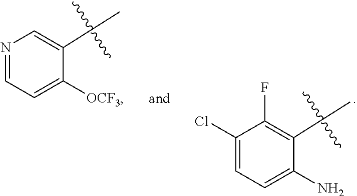 [SMILES: *c1cnccc1OC(F)(F)F ; *c1c(N)ccc(Cl)c1F]

In embodiments, R² is —OR^{12′}.

In embodiments, is selected from

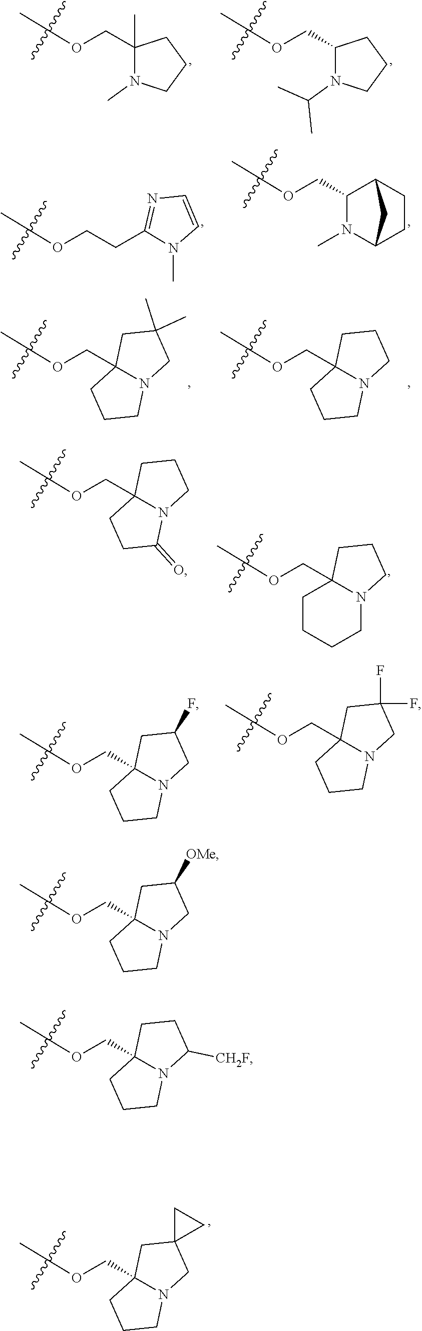

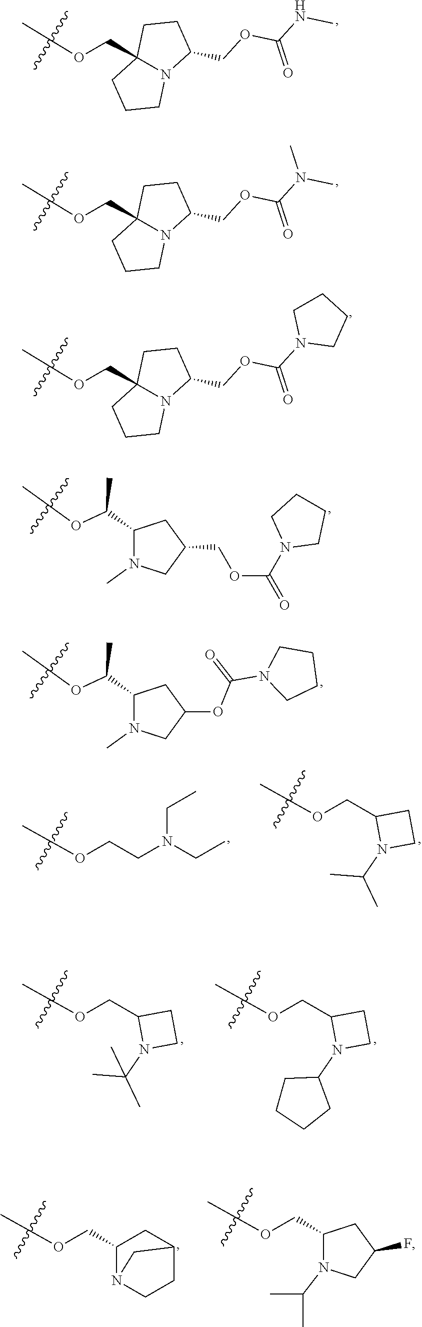

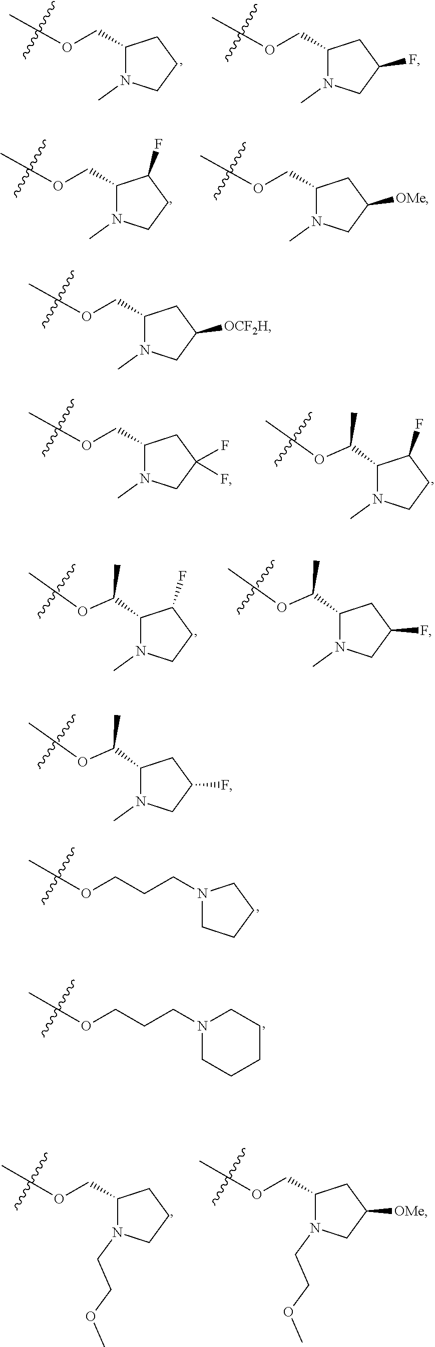

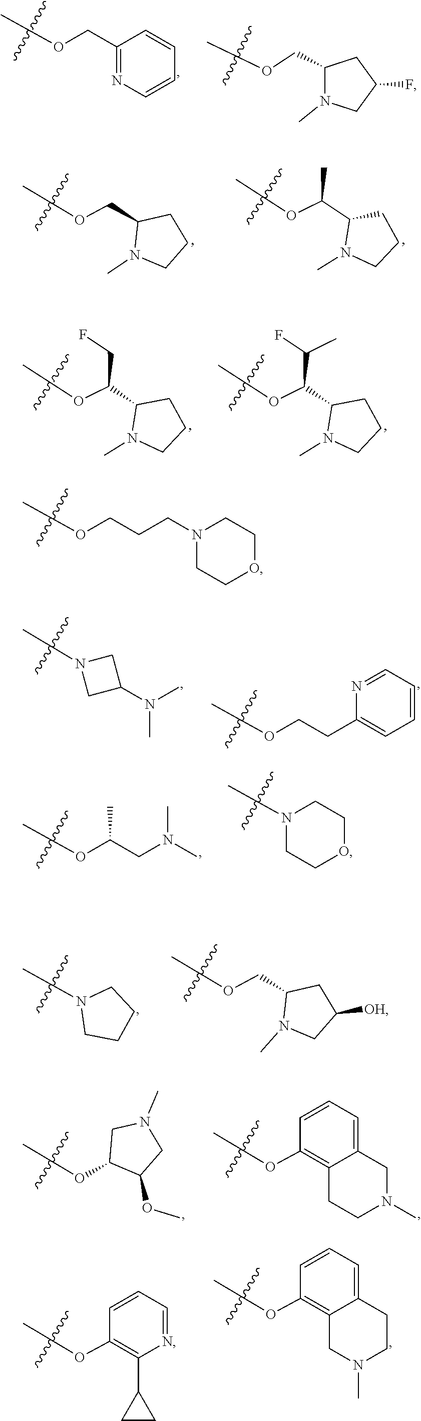

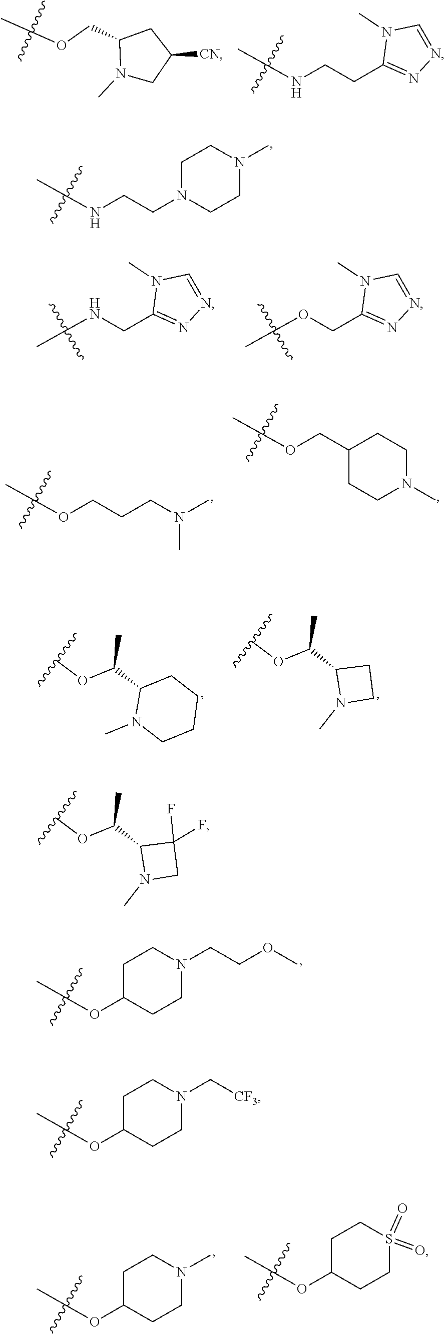

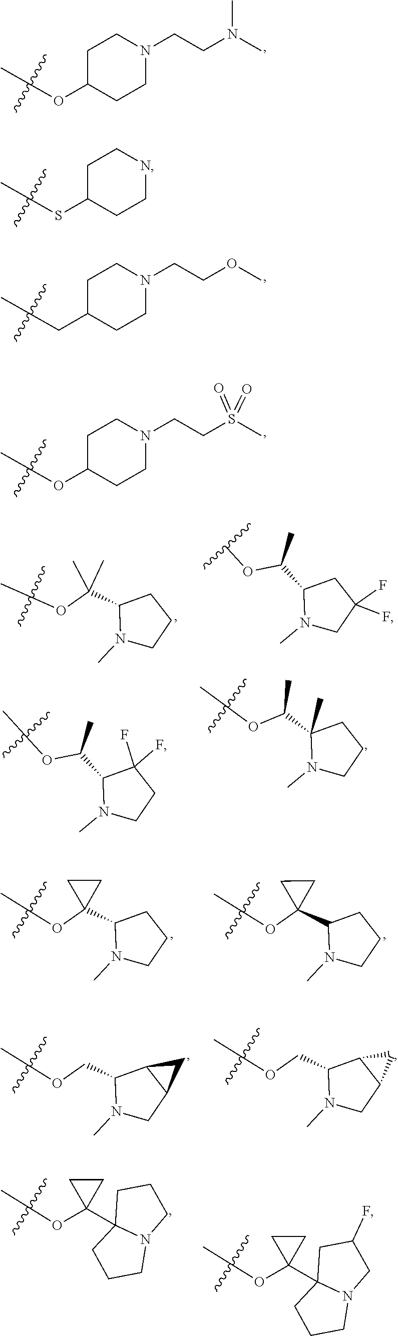

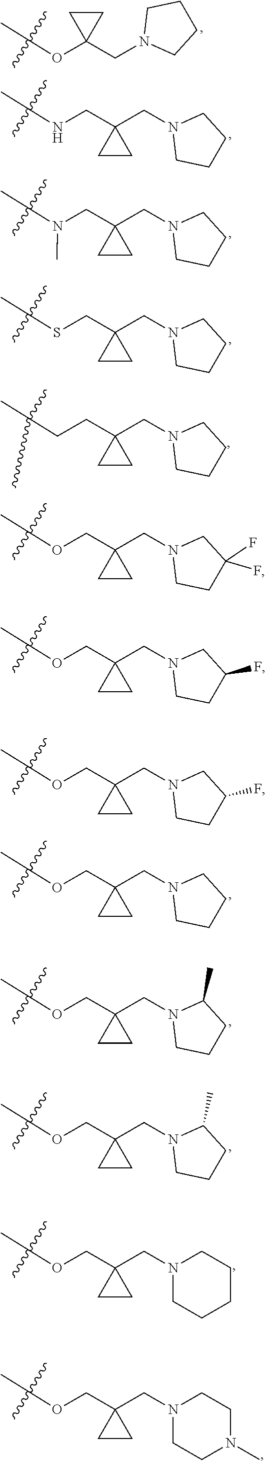

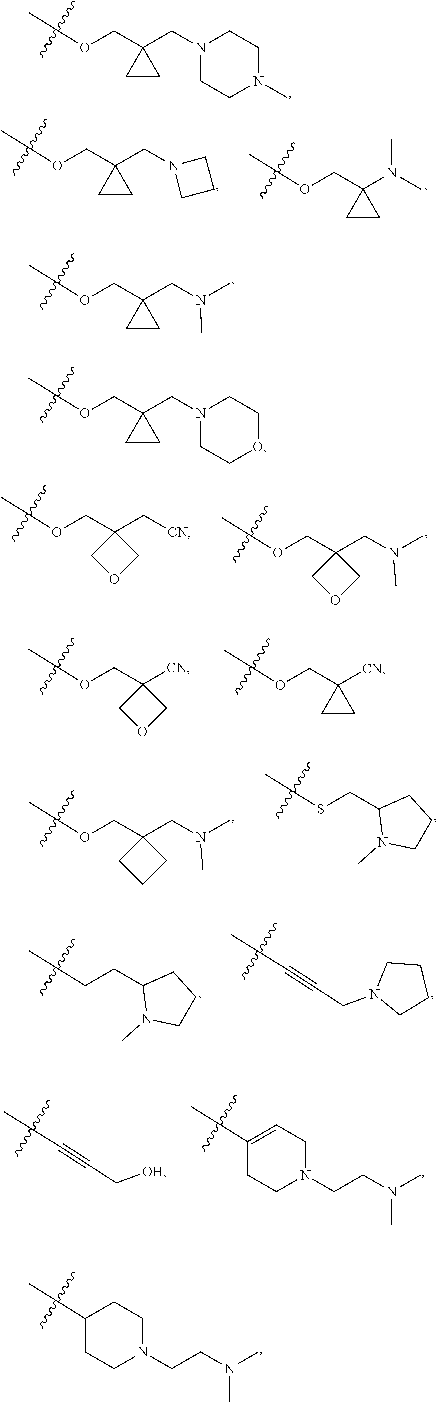

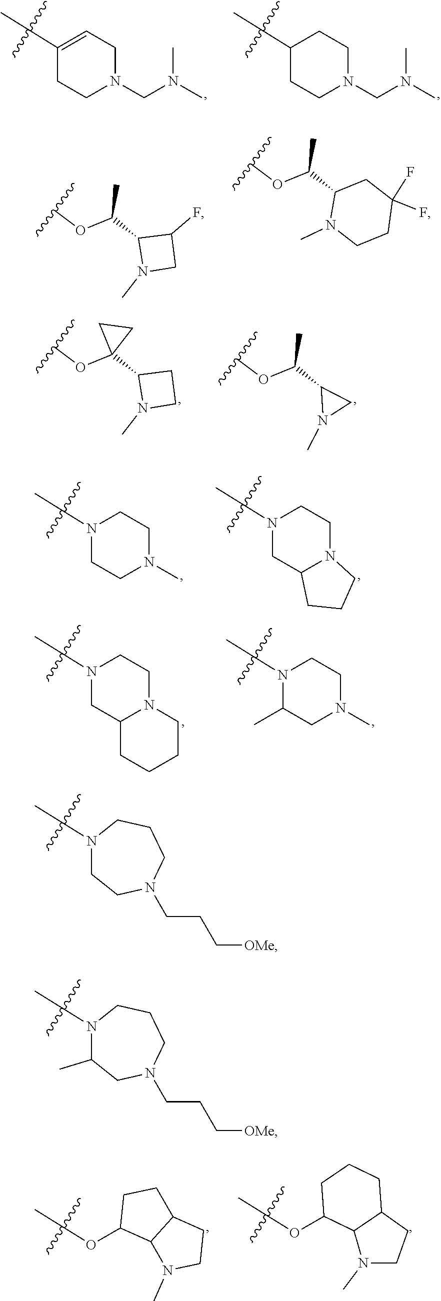

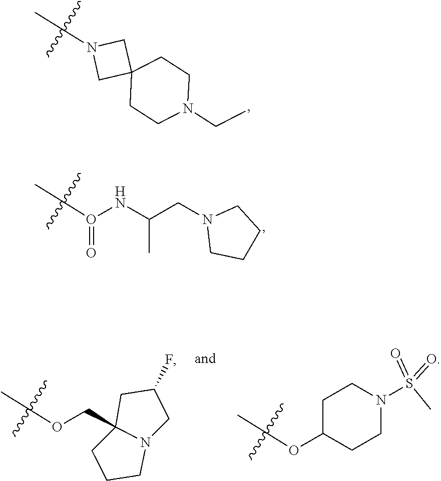

In an aspect is provided a compound having the formula A-L^{AB}-B wherein
A is a monovalent form of a compound described herein;
L^{AB} is a covalent linker bonded to A and B; and
B is a monovalent form of a degradation enhancer.

In embodiments of a compound described herein, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885.

In embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1.

In embodiments, L^{AB} is -L^{AB1}-L^{AB2}-L^{AB3}-L^{AB4}-L^{AB5}-;
L^{AB1}, L^{AB2}, L^{A}B3, L^{AB4}, and L^{AB5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆alkylene, (—O—C₁₋₆alkyl)₂-, (—C₁₋₆alkyl-O)₂—, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁heteroarylene, wherein C₁₋₆ alkylene, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁ heteroarylene, are optionally substituted with one, two, or three R^{20j}; wherein each C₁₋₆alkyl of (—O—C₁₋₆alkyl)₂- and (—C₁₋₆ alkyl-O)₂— is optionally substituted with one, two, or three R^{20j};
z is independently an integer from 0 to 10;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
each R^{20d}, R^{20e}, R^{20f}, and R^{20j} are each independently selected from halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl.

In embodiments, L^{AB} is —(O—C₂alkyl)_{z}- and z is an integer from 1 to 10. In embodiments, L^{AB} is —(C₂alkyl-O—)_{z}— and z is an integer from 1 to 10. In embodiments, L^{AB} is —(CH₂)_{zz1}L^{AB2}(CH₂O)_{zz2}—, wherein L¹² is a bond, a 5 or 6 membered heterocycloalkylene or heteroarylene, phenylene, —(C₂-C₄)alkynylene, —SO₂— or —NH—; and zz1 and zz2 are independently an integer from 0 to 10. In embodiments, L^{AB} is —(CH₂)_{zz1}(CH₂O)_{zz2}—, wherein zz1 and zz2 are each independently an integer from 0 to 10. In embodiments, L^{AB} is a PEG linker.

In embodiments, B is a monovalent form of a compound selected from

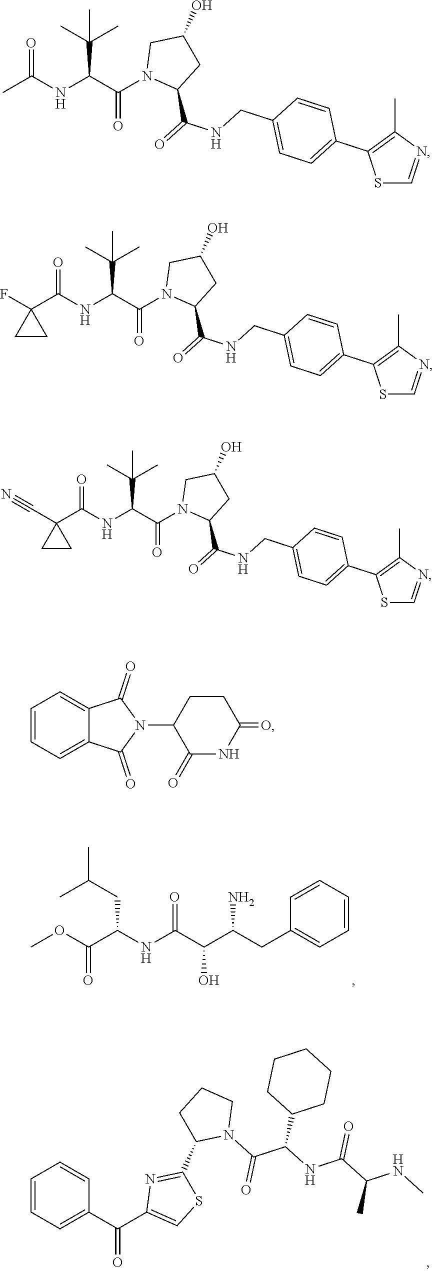 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)(C)C)cc1 ; Cc1ncsc1-c1ccc(CNC(=O)[C@@H]2C[C@@H](O)CN2C(=O)[C@@H](NC(=O)C2(C#N)CC2)C(C)(C)C)cc1 ; O=C1CCC(N2C(=O)c3ccccc3C2=O)C(=O)N1 ; COC(=O)[C@H](CC(C)C)NC(=O)[C@@H](O)[C@H](N)Cc1ccccc1 ; CN[C@@H](C)C(=O)N[C@H](C(=O)N1CCC[C@H]1c1nc(C(=O)c2ccccc2)cs1)C1CCCCC1]

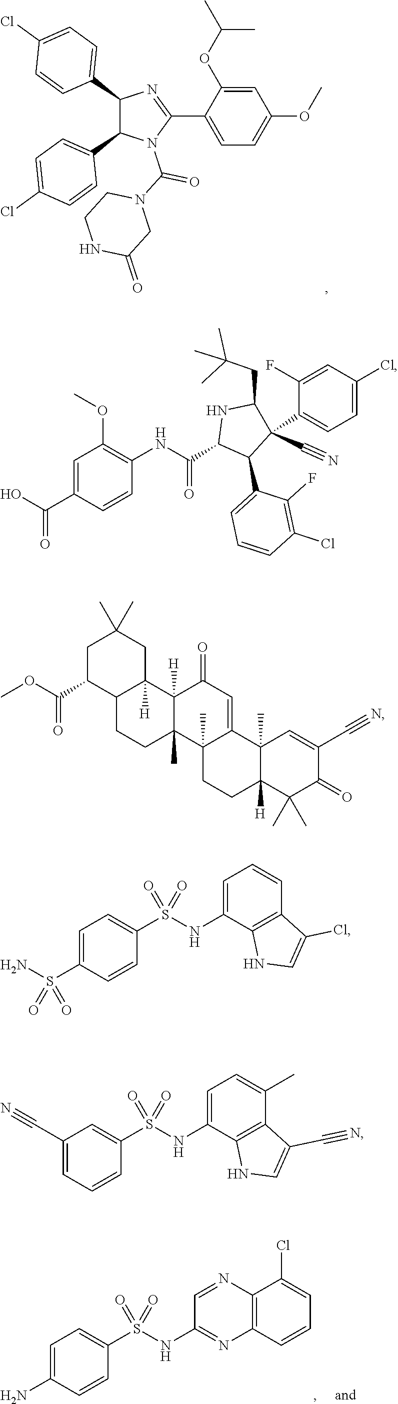

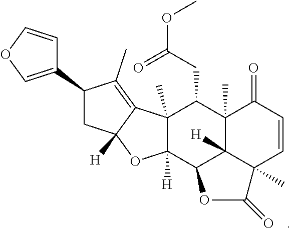

In an aspect is provided a compound of Formula (I-3), or a pharmaceutically acceptable salt or solvate thereof:

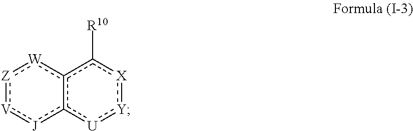

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), S(O), and S(O)₂;
s1 is an integer from 1 to 6;
s2 is an integer from 0 to 3;
s1 and s2 are not both 1; s1 and s2 are not both 2; and
all other variables of the formula (e.g., W, Z, V, J, Y, U, R¹⁰, L⁷, R⁷, W¹, W³, W⁵, s3, R¹, R⁶, L², R⁴, R⁵, R², R^{4c}, R^{4d}, R⁸, R^{8a}, R^{8b}, R¹⁷, R^{17b}, L¹, L^{1b}, R^{1c}, R^{1f}, R^{1g}, R^{1e}, R¹⁹, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (I), or embodiments thereof.

In an aspect is provided a compound of Formula (I-4), or a pharmaceutically acceptable salt or solvate thereof:

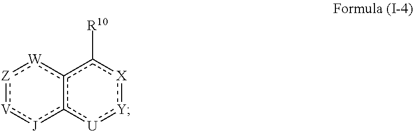

Wherein
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), ═C(R^{21b})₂, —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl), —S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl)-S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁ heteroaryl are optionally substituted with one, two, or three R^{20k};
R² is selected from halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉ heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₄₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₄₋₉heterocycloalkyl, C₆₋₁₀aryl, and C₁₋₉heteroaryl are optionally substituted with one, two, or three R^{20d};
each R^{20a}, R^{20b}, R^{20c}, R^{20d}, R^{20e}, R^{20f}, R^{20g}, R^{20h}, R²⁰ⁱ, R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), ═C(R^{21b})₂, —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²¹, —N(R²⁴)S(O)₂R²⁵, —C(O)R²¹, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
[symbol] indicates the location of attachment of the depicted chemical formula or atom to a substituent, a further component of a molecule, or an atom; and
R^{21b} is independently selected at each occurrence from hydrogen, halogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, or two R^{21b} are taken together with the carbon atom to which they are attached to form C₃₋₁₀cycloalkyl or C₂₋₉heterocycloalkyl; each of which is optionally substituted with one, two, or three substituents independently selected from halogen, C₁-3 alkyl, C₁-3 haloalkyl, and —OH;
[symbol] indicates a single or double bond such that all valences are satisfied; and
all other variables of the formula (e.g., W, Z, V, J, Y, U, R¹⁰, L⁷, R⁷, W¹, W³, W², W⁴, W⁵, s1, s2, s3, R¹, R⁶, L², R^{4c}, R^{4d}, R⁸, R^{8a}, R^{8b}, R¹⁷, R^{17b}, L¹, L^{1b}, R^{1e}, R^{1f}, R^{1g}, R^{1c}, R¹⁹, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R²¹, R²², R²³, R²⁴, and R²⁵) are as described for Formula (I-3), or embodiments thereof.

In an aspect is provided a compound of Formula (II-3), or a pharmaceutically acceptable salt or solvate thereof:

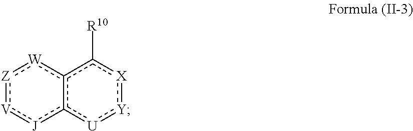

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
s1 is an integer from 1 to 6;
s2 is an integer from 0 to 3; and
all other variables of the formula (e.g., W, Z, V, J, U, Y, R¹⁰, L⁷, L², R⁷, R⁵, R¹⁹, R², W¹, W³, W², W⁵, s3, R¹, R⁴, R⁶, R⁸, R^{8a}, R^{8b}, R¹⁷, R^{17b}, L¹, L^{1b}, R^{1e}, R^{1f}, R^{1g}, R¹⁰, R¹ⁱ, R¹⁶, R^{16a}, R¹⁶, R^{12c}, R^{12b}, R^{12b}, X, R³, R¹², R^{12′}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (II), or embodiments thereof.

In an aspect is provided a compound of Formula (II′), or a pharmaceutically acceptable salt or solvate thereof:

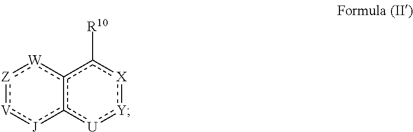

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
s1 is an integer from 1 to 6;
s2 is an integer from 0 to 3;
s3 is an integer from 1 to 3;
Y is N, C(R²), C(R²)(R^{2c}), N(R^{2b}), S(O), S(O)₂, or C(O);
R¹⁰ is -L⁷-R⁷;
R⁷ is

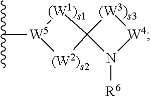

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R¹⁹ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl), —S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R¹ⁱ;
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates the location of attachment of the depicted chemical formula or atom to a substituent, a further component of a molecule, or an atom;
[symbol] indicates a single or double bond such that all valences are satisfied; and
all other variables of the formula (e.g., W, Z, V, J, U, L⁷, W¹, W³, W², W⁵, R¹, R⁴, R⁶, R⁸, R^{8a}, R^{8b}, R¹⁷, R¹⁷, L¹, L^{1b}, R^{1e}, R^{1f}, R^{1g}, R^{1c}, R¹, R¹⁶, R^{1a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R^{20l}, R^{21b}, R²², R²³, R²⁴, and R²⁵) are as described for Formula (I-4), or embodiments thereof.

In an aspect is provided a compound of Formula (II″), or a pharmaceutically acceptable salt or solvate thereof:

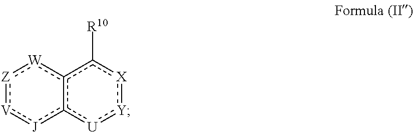

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
s1 is an integer from 1 to 6;
s2 is an integer from 0 to 3;
s3 is an integer from 1 to 3;
Y is N, C(R²), C(R²)(R^{2c}), N(R^{2b}), S(O), S(O)₂, or C(O);
R¹⁰ is -L⁷-R⁷;
R⁷ is

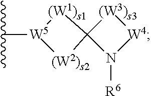

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl), —S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R¹ⁱ;
R² is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates the location of attachment of the depicted chemical formula or atom to a substituent, a further component of a molecule, or an atom; and [symbol] indicates a single or double bond such that all valences are satisfied.
all other variables of the formula (e.g., W, Z, V, J, U, L⁷, W¹, W³, W², W⁵, R¹, R⁴, R⁶, R⁸, R^{8a}, R^{8b}, R¹⁷, R^{17a}, L¹, L^{1b}, R^{1e}, R^{1f}, R^{1g}, R^{1c}, R¹ⁱ, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R²⁰, R²¹, R^{21b}, R²², R²³, R²⁴, and R²⁵) are as described for Formula (I-4), or embodiments thereof.

In an aspect is provided a compound of Formula (IV-3), or a pharmaceutically acceptable salt or solvate thereof:

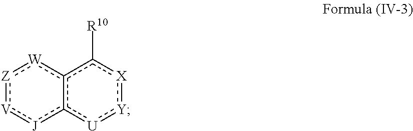

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), and C(R⁴)(R⁴);
s1 is an integer from 1 to 6;
s2 is an integer from 0 to 3;
s1+s2 is not equal to 2 or 4;
R¹⁹ is selected from a C₂₋₁₁heterocycloalkyl and C₂₋₁₂heteroaryl, wherein the C₂₋₁₁heterocycloalkyl and C₂₋₁₂heteroaryl are optionally substituted with one, two, three, four, five, six, or seven R²⁰ⁱ;
R² is hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d}; and
all other variables of the formula (e.g., W, Z, V, J, Y, U, R¹⁰, L⁷, R⁷, W¹, W³, W², W⁵, s3, R¹, R⁶, L², R⁴, R⁵, R^{4c}, R^{4d}, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R²⁰, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (IV), or embodiments thereof.

In an aspect is provided a compound of Formula (IV-4), or a pharmaceutically acceptable salt or solvate thereof:

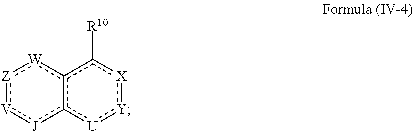

wherein
Y is N, C(R²), C(R²)(R^{2c}), N(R^{2b}), S(O), S(O)₂, or C(O);
R¹⁰ is -L⁷-R⁷;
R⁷ is

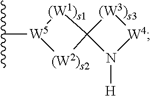

W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), or C(R⁴)(R⁴);
s1 is an integer from 1 to 6 and s2 is an integer selected from 2 and 3; or s1 is an integer from 2 to 6 and s2 is an integer from 1 to 3;
s1+s2 is not equal to 2 or 4;
R¹⁹ is selected from a C₂₋₁₁heterocycloalkyl and C₂₋₁₂heteroaryl, wherein the C₂₋₁₁heterocycloalkyl and C₂₋₁₂heteroaryl are optionally substituted with one, two, three, four, five, six, or seven R¹ⁱ;
R² is hydrogen, halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C_{4- 9}heterocycloalkyl)-R^{12b}, —(C₆₋₁₀aryl)-R^{12b}, or —(C₁₋₉heteroaryl)-R^{2b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates a single or double bond such that all valences are satisfied; and
all other variables of the formula (e.g., W, Z, V, J, U, L⁷, W¹, W³, W⁵, s3, R¹, R⁴, R⁸, R^{8a}, R^{8b}, R¹⁷, R¹⁷, L¹, L^{1b}, R^{1e}, R^{1f}, R^{1g}, R¹⁰, R¹¹, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

As applied to compounds according to any of the formulae provided herein, in some embodiments, s1 is 1 and s2 is 0, 2, or 3. In some embodiments, s1 is 2 and s2 is 0, 1, or 3. In some embodiments, s1 is 2 and s2 is 1 or 3. In some embodiments, s1 is 3 and s2 is 0 to 3. In some embodiments, s1 is 4 and s2 is 0 to 3. In some embodiments, s1 is 5 and s2 is 0 to 3. In some embodiments, s1 is 1, s2 is 1, and s3 is 1, 2, or 3. In some embodiments, s2 is 1 or 2. In some embodiments, s2 is 3. In some embodiments, s1 is 2, s2 is 1, and s3 is 1. In some embodiments, s2 is 1, 2 or 3 and s3 is 1, 2, or 3. In some embodiments, s2 is 1, and s3 is 1, 2, or 3.

In an aspect is provided a pharmaceutical composition comprising a compound described herein, or a pharmaceutically acceptable salt or solvate thereof, and a pharmaceutically acceptable excipient.

In an aspect is provided a method of treating cancer in a subject in need thereof, comprising administering to the subject a therapeutically effective amount of a compound described herein, or a pharmaceutically acceptable salt or solvate thereof.

In an aspect is provided a method of treating cancer in a subject comprising a Ras mutant protein, the method comprising: modifying the Ras mutant protein of said subject by administering to said subject a compound, wherein the compound is characterized in that upon contacting the Ras mutant protein, said Ras mutant protein is modified covalently at a residue corresponding to reside 12 of SEQ ID No: 1, such that said modified Ras mutant protein exhibits reduced Ras signaling output.

In embodiments, the cancer is a solid tumor. In embodiments, the cancer is a hematological cancer.

In an aspect is provided a method of modulating signaling output of a Ras protein, comprising contacting a Ras protein with an effective amount of a compound described herein, or a pharmaceutically acceptable salt or solvate thereof, thereby modulating the signaling output of the Ras protein.

In an aspect is provided a method of inhibiting cell growth, comprising administering an effective amount of a compound described herein, or a pharmaceutically acceptable salt or solvate thereof, to a cell expressing a Ras protein, thereby inhibiting growth of said cells.

In embodiments, the method includes administering an additional agent. In embodiments, the additional agent comprises (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR⁸, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of SHP pathway including SHP2 inhibitor (e.g., 6-(4-amino-4-methylpiperidin-1-yl)-3-(2,3-dichlorophenyl)pyrazin-2-amine, as well as SHP1 inhibitors, or (33) an inhibitor of Src, FLT3, HDAC, VEGFR, PDGFR, LCK, Bcr-Abl or AKT or (34) an inhibitor of KrasG12C mutant (e.g., including but not limited to AMG510, MRTX849, and any covalent inhibitors binding to the cysteine residue 12 of Kras, the structures of these compounds are publicly known) (e.g., an inhibitor of Ras G12C as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety), (35) a SHC inhibitor (e.g., PP2, AID371185), (36) a GAB inhibitor (e.g., GAB-0001), (37) a GRB inhibitor, (38) a PI-3 kinase inhibitor (e.g., Idelalisib, Copanlisib, Duvelisib, Alpelisib, Taselisib, Perifosine, Buparlisib, Umbralisib, NVP-BEZ235-AN), (39) a MARPK inhibitor, (40) CDK4/6 (e.g., palbociclib, ribociclib, abemaciclib), or (41) MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197), or (42) an inhibitor of SHP pathway including SHP2 inhibitor (e.g., 6-(4-amino-4-methylpiperidin-1-yl)-3-(2,3-dichlorophenyl)pyrazin-2-amine, RMC-4630,

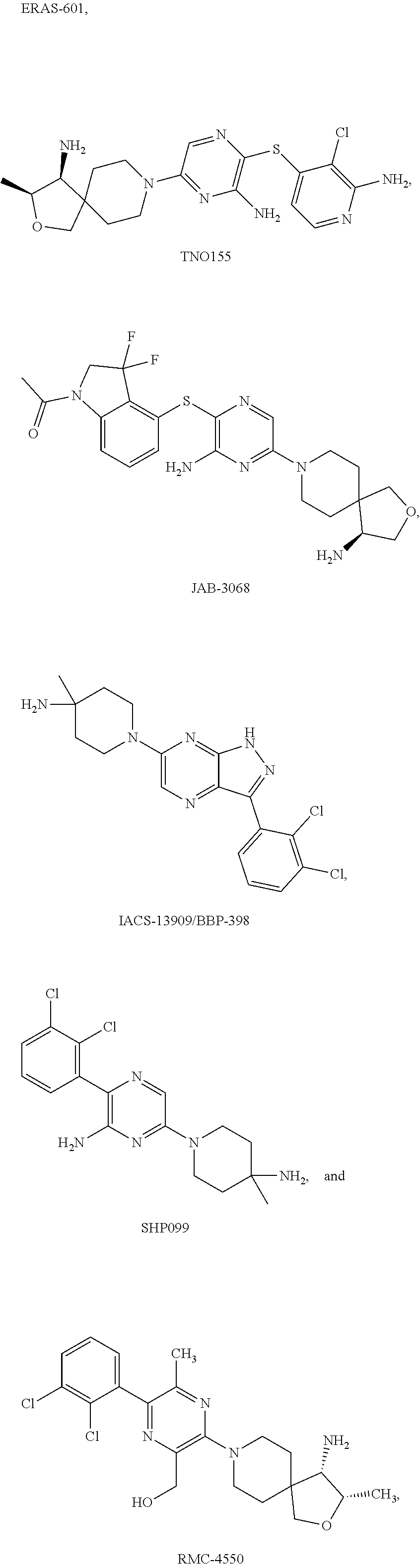

or a SHP1 inhibitor; (43) checkpoint immune blockade agents (e.g., anti-PD-1 and/or anti-PD-L1 antibody, including but not limited to Nivolumab, Pembrolizumab, Cemiplimab, Durvalumab, as well as anti-CLTA-4 antibody, including Ipilimurmab).

In embodiments, the additional agent comprises an inhibitor of SHP2 selected from RMC-4630, ERAS-601,

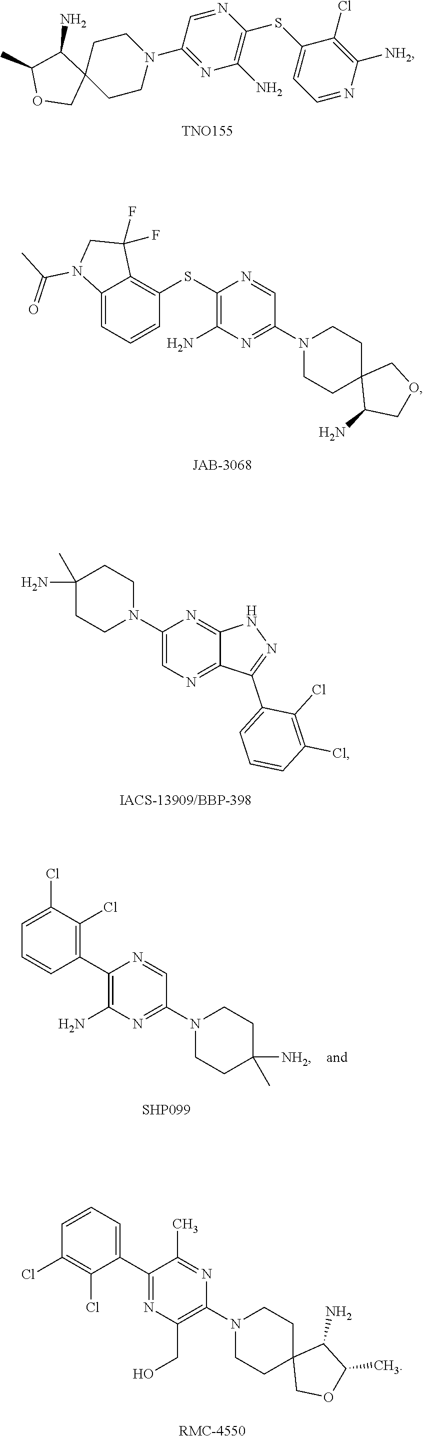

In embodiments, the additional agent comprises an inhibitor of SOS selected from

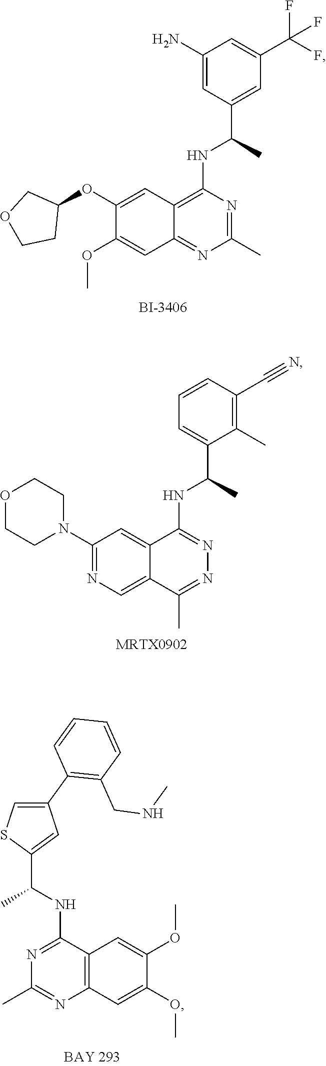

### RMC-5845, and BI-1701963.

In embodiments, the additional agent comprises an inhibitor of EGFR selected from afatinib, erlotinib, gefitinib, lapatinib, cetuximab panitumumab, osimertinib, olmutinib, and EGF-816. In embodiments, the additional agent comprises an inhibitor of MEK selected from trametinib, cobimetinib, binimetinib, selumetinib, refametinib, and AZD6244. In embodiments, the additional agent comprises an inhibitor of ERK selected from ulixertinib, MK-8353, LTT462, AZD0364, SCH772984, BIX02189, LY3214996, and ravoxertinib. In embodiments, the additional agent comprises an inhibitor of CDK4/6 selected from palbociclib, ribociclib, and abemaciclib. In embodiments, the additional agent comprises an inhibitor of BRAF selected from Sorafenib, Vemurafenib, Dabrafenib, Encorafenib, regorafenib, and GDC-879.

In an aspect is provided a compound of Formula (I), or a pharmaceutically acceptable salt or solvate thereof:

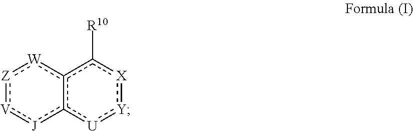

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), or S(O)₂;
U is N, C(R^{2c}), C(R^{2c})(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};

R⁷ is

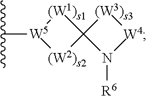

each W¹ and each W³ are independently selected from N(R¹), N(R⁴), C(R¹)(R¹), C(R¹)(R⁴), C(R⁴)(R⁴), C(O), S, O, S(O), and S(O)₂;
each W² is independently selected from N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), S(O), and S(O)₂;
W⁵ is selected from N, C(R¹), and C(R⁴);
s1 is 1 and s2 is 0, 2, or 3; s1 is 2 and s2 is 0, 1, or 3; s1 is 3 and s2 is 0 to 3; s1 is 4 and s2 is 0 to 3; s1 is 5 and s2 is 0 to 3; or s1 is 6 and s2 is 0 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵ and wherein R⁶ is optionally capable of forming a covalent bond with a Ras amino acid;
L² is a bond, —O—, —N(R^{4d})—, —C(O)—, —S—, —S(O)₂—, —S(O)—, —P(O)R^{4d}—, —CR^{4c}R^{4c}, —OCR^{4c}R^{4c}—, —N(R^{4d})CR^{4c}R^{4c}—, —C(O)CR^{4c}R^{4c}—, —SCR^{4c}R^{4c}—, —S(O)₂CR^{4c}R^{4c}—, —S(O)CR^{4c}R^{4c}—, —P(O)R^{4d}CR^{4c}R^{4c}—, —CR^{4c}R^{4c}R^{4c}R^{4c}, —CR^{4c}R^{4c}O—, —CR^{4c}R^{4c}N(R^{4d})—, —CR^{4c}R^{4c}C(O)—, —CR^{4c}R^{4c}S—, —CR^{4c}R^{4c}S(O)₂—, —CR^{4c}R^{4c}S(O)—, —CR^{4c}R^{4c}P(O)R^{4d}—, —N(R^{4d})C(O)—, —N(R^{4d})S(O)₂—, —N(R^{4d})S(O)—, —N(R^{4d})P(O)R^{4d}—, —C(O)N(R^{4d})—, —S(O)₂N(R^{4d})—, —S(O)N(R^{4d})—, —P(O)R^{4d}N(R^{4d})—, —OC(O)—, —OS(O)₂—, —OS(O)—, —OP(O)R^{4d}—, —C(O)O—, —S(O)₂O—, —S(O)O—, —P(O)R^{4d}O—, —CR^{4c}R^{4c}R^{4c}CR^{4c}R^{4c}—, —OCRR^{4c}R^{4c}R^{4c}—, —N(R^{4d})CR^{4c}R^{4c}R^{4c}R^{4c}—, —C(O)CR^{4c}R^{4c}CR^{4c}R^{4c}—, —SCR^{4c}R^{4c}R^{4c}R^{4c}—, —S(O)₂CR^{4c}R^{4c}CR^{4c}R^{4c}—, —S(O)CR^{4c}R^{4c}CR^{4c}R^{4c}—, —P(O)R^{4d}CR^{4c}R^{4c}CR^{4c}R^{4c}—, —CR^{4c}R^{4c}R^{4c}R^{4c}O—, —CR^{4c}R^{4c}CR^{4c}R^{4c}N(R^{4d})—, —CR^{4c}R^{4c}R^{4c}R^{4c}C(O)—, —CR^{4c}R^{4c}CR^{4c}R^{4c}S—, —CR^{4c}R^{4c}R^{4c}R^{4c}S(O)₂—, —CR^{4c}R^{4c}R^{4c}R^{4c}S(O)—, or —CR^{4c}R^{4c}R^{4c}R^{4c}P(O)R^{4d}—;
each R^{4c} is independently selected from hydrogen, halogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴)₉, —OCH₂C(O)OR¹⁴, —OC(O)R^{14a}, —N(R¹⁴)(R¹⁴), —N(R¹⁴)C(O)N(R¹⁴)(R¹⁴), —N(R¹⁴)C(O)OR¹⁴, —N(R¹⁴)C(O)R^{14a}, and —N(R¹⁴)S(O)₂R¹⁴, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
each R^{4d} is independently selected from hydrogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, and —OC(O)R^{14a}, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R¹⁷ is -L¹-R¹⁹;
R^{17b} is -L^{1b}-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R¹⁴), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R¹ (R^{1a})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰;
R^{2c} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R^{20c}, R^{20d}, R^{20e}, R^{20f}, R^{20g}, R^{20h}, R²⁰ⁱ, R^{20k}, and R^{20m} are each independently selected from halogen, oxo, —CN, C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.

A compound of Formula (II), or a pharmaceutically acceptable salt or solvate thereof:

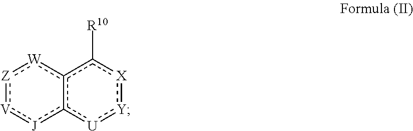

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R¹⁷), and N(R^{16b}); wherein exactly one of V and J is C(R¹⁷), C(R¹⁷)(R^{16a}), or N(R^{17b});
Y is N, C(R²), C(R²)(R¹), N(R^{2b}), S(O), S(O)₂, or C(O);
U is N, C(R¹), C(R¹)(R¹), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is

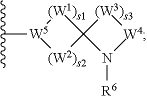

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵ and wherein R⁶ is optionally capable of forming a covalent bond with a Ras amino acid;
L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁ heterocycloalkyl, C₆₋₁₂aryl, and C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹², —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{2c};
R¹⁷ is -L¹-R¹⁹;
R^{17b} is -L^{1b}-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R^{1f})(R^{1g})N(R^{1c}), and C(R¹)(R¹⁹); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, three, four, five, six, or seven R¹ⁱ
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2e} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R^{20c}, R^{20d}, R^{20e}, R^{20f}, R^{20g}, R^{20h}, R², R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.

In embodiments, L² is —C(O)—. In embodiments, R⁵ is a 5 or 6 membered partially unsaturated heterocycloalkyl or a 5 or 6 membered heteroaryl, each optionally substituted with one, two or three R^{20k}, wherein the partially unsaturated 5 or 6 membered heterocycloalkyl or 5 or 6 membered heteroaryl comprises one, two, or three ring nitrogen atoms; and each is bonded to L² through a ring nitrogen. In embodiments, R⁵ is a 5 membered heteroaryl optionally substituted with one, two or three R^{20k}, wherein the 5 membered heteroaryl comprises one, two, or three ring nitrogen atoms; and the 5 membered heteroaryl is bonded to L² through a ring nitrogen.

In embodiments, L² is —C(O)—; and R⁵ is a C₃₋₁₂cycloalkyl optionally substituted with one, two or three R^{20k}. In embodiments, L² is —C(O)—; and R⁵ is a cyclopropyl optionally substituted with one, two or three R^{20k} selected from halogen and CN. In embodiments, L² is —C(O)—; and R⁵ is C₂₋₆alkenyl, wherein C₂₋₆alkenyl is optionally substituted with one, two, or three R^{20k}. In embodiments, L² is —C(O)—; and R⁵ is C₂₋₆alkynyl, wherein C₂₋₆alkynyl is optionally substituted with one, two, or three R^{20k}.

In embodiments, Y is C(O). In embodiments, Y is C(R²). In embodiments, X is N. In embodiments, X is C(R³). In embodiments, U is N. In embodiments, U is N(R^{2b}). In embodiments, W is a C(R¹⁸). In embodiments, W is a CH. In embodiments, W is a C(O). In embodiments, Z is N. In embodiments, Z is C(R⁸). In embodiments, Z is C(Cl). In embodiments, Z is N(R^{8b}). In embodiments, Z is N(cyclopropyl). In embodiments, V is C(R¹⁷). In embodiments, J is C(R¹⁶). In embodiments, J is C(F). In embodiments, L⁷ is a bond. In embodiments, W¹ is C(R⁴)₂. In embodiments, W¹ is independently selected from CH₂ and CH(CH₃). In embodiments, W¹ is independently selected from CH₂ and CH(CH₂CN). In embodiments, W¹ is CH₂. In embodiments, W³ is CH₂. In embodiments, W² is C(R⁴)₂. In embodiments, W² is independently selected from CH₂, CH(CH₂CN), CH(CHF₂), CH(CH₂CH₃), and CH(CH₃). In embodiments, W² is CH₂. In embodiments, W² is independently selected from CH(CH₂CH₃) and CH(CH₃). In embodiments, W⁴ is CH₂.

In embodiments, W⁵ is N. In embodiments, W⁵ is CH. In embodiments, s1 is 2. In embodiments, s1 is 3. In embodiments, s1 is 4. In embodiments, s2 is 1. In embodiments, s2 is 2. In embodiments, s3 is 1.

As applied to compounds according to any of the formulae provided herein, in some embodiments, s1 is 1 and s2 is 0, 2, or 3. In some embodiments, s1 is 2 and s2 is 0, 1, or 3. In some embodiments, s1 is 2 and s2 is 1 or 3. In some embodiments, s1 is 3 and s2 is 0 to 3. In some embodiments, s1 is 4 and s2 is 0 to 3. In some embodiments, s1 is 5 and s2 is 0 to 3. In some embodiments, s1 is 1, s2 is 1, and s3 is 1, 2, or 3. In some embodiments, s2 is 1 or 2. In some embodiments, s2 is 3. In some embodiments, s1 is 2, s2 is 1, and s3 is 1. In some embodiments, s2 is 1, 2 or 3 and s3 is 1, 2, or 3. In some embodiments, s2 is 1, and s3 is 1, 2, or 3.

In embodiments, R⁷ is selected from

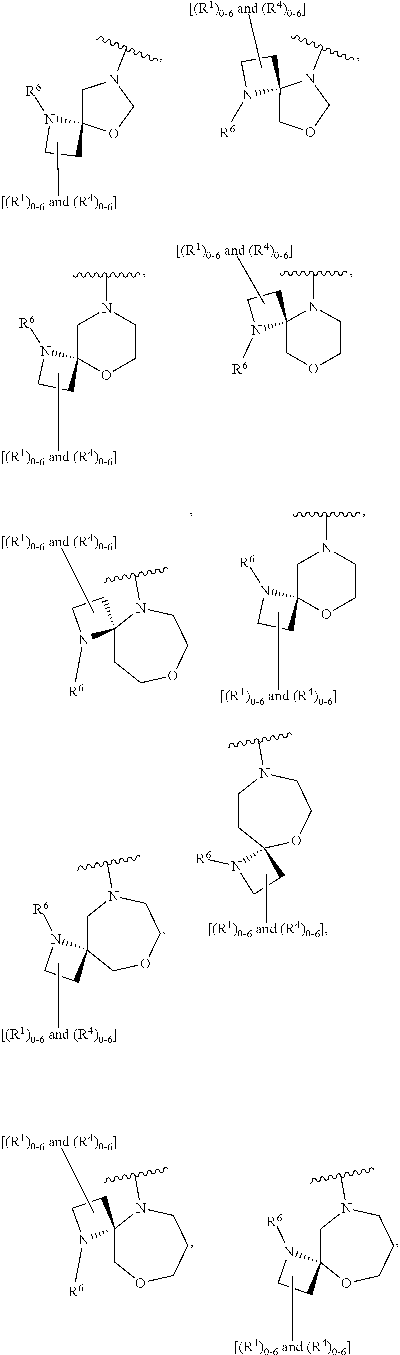

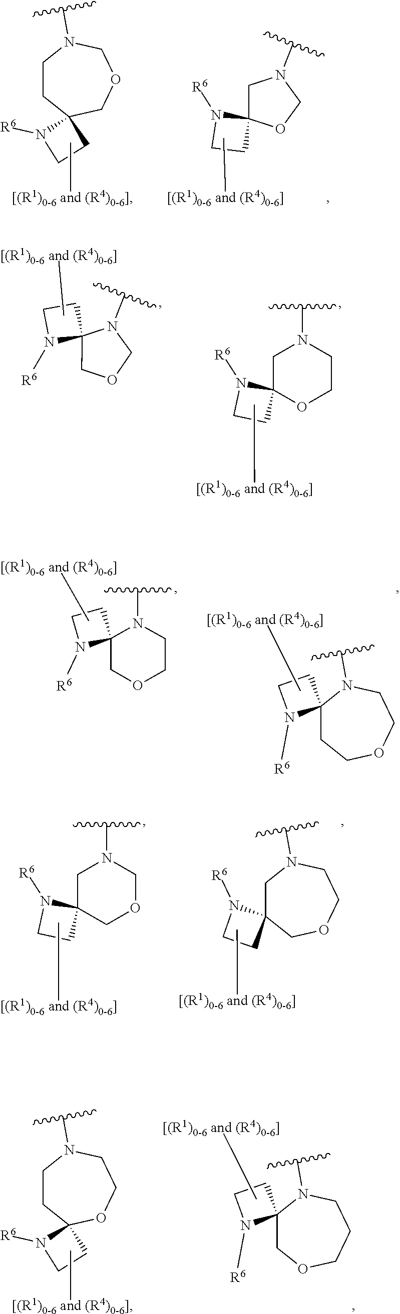

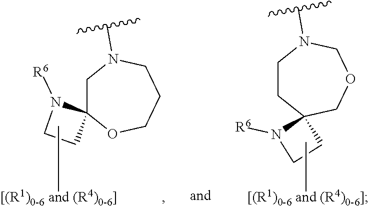

wherein R¹ and R⁴ substituents may be bonded to either spirocyclic ring.

In embodiments, R⁷ is selected from

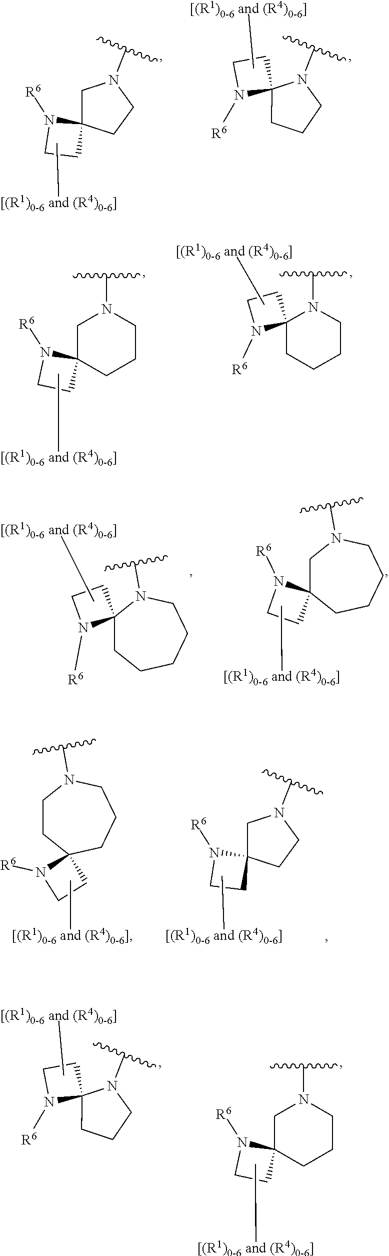

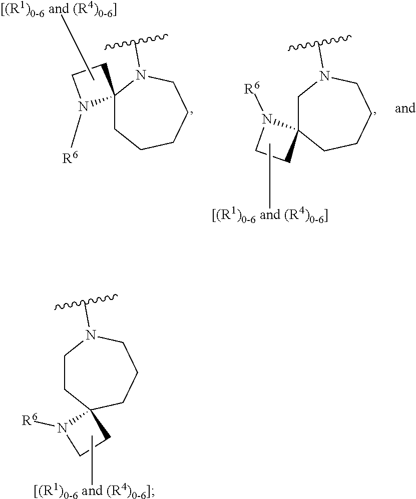

wherein R¹ and R⁴ substituents may be bonded to either spirocyclic ring.

In embodiments, R⁷ is selected from

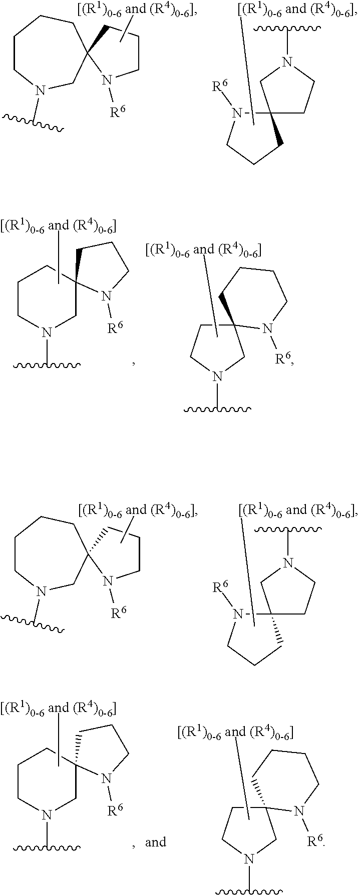

In embodiments, R⁷ is

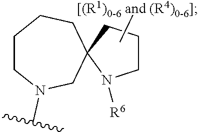

wherein R¹ and R⁴ substituents may be bonded to either spirocyclic ring.

In embodiments, R¹⁷ is -L¹-R¹⁹ or -L^{1b}-R¹⁹; L¹ is a bond, and Lib is a bond.

In embodiments, R¹⁹ is:

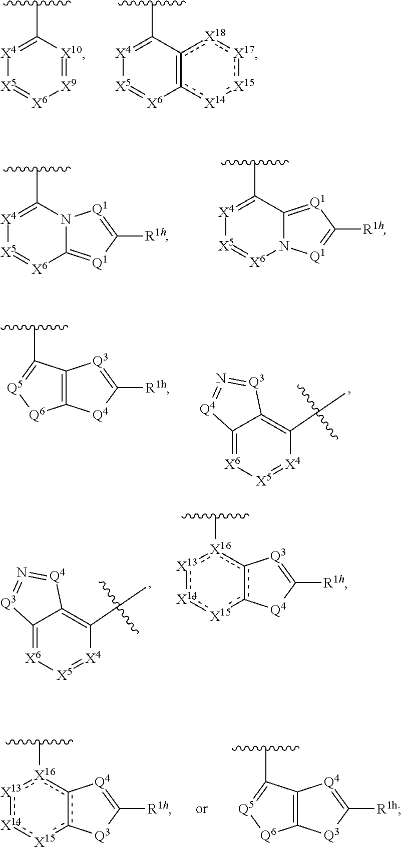

Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, X¹⁸ are independently selected from a C(O), C(R^{1a}), N, C(R^{1a})(R^{1b}), and N(R^{1c});
X¹⁶ is selected from C, N, and C(R^{1a});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments, R¹⁹ is selected from

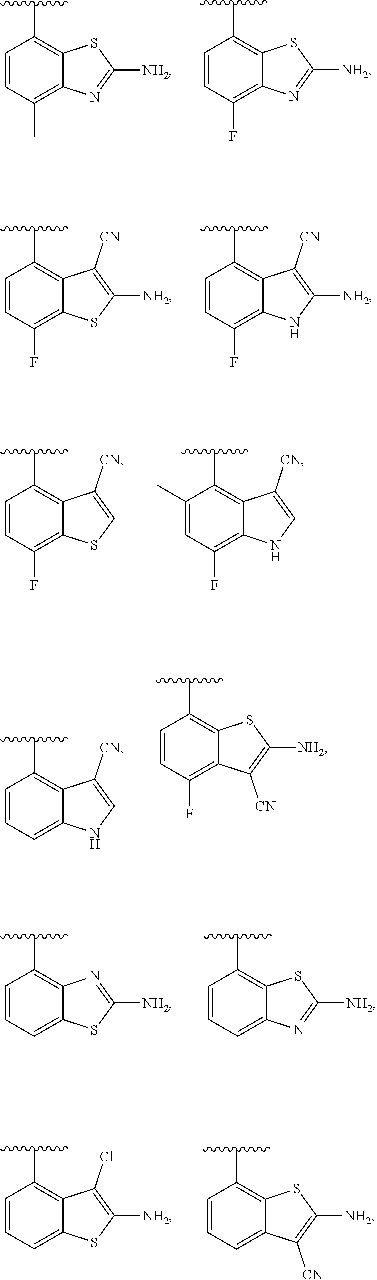

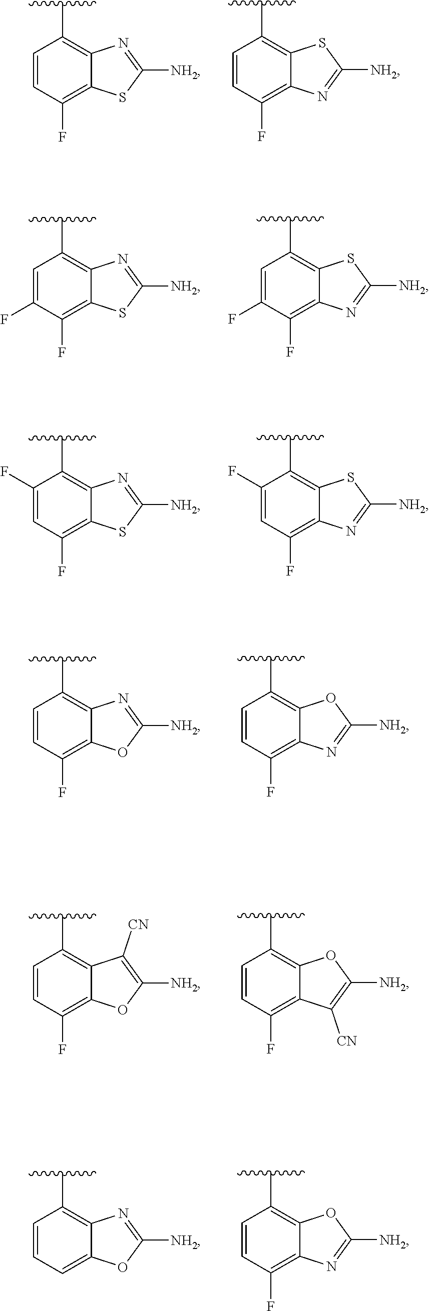

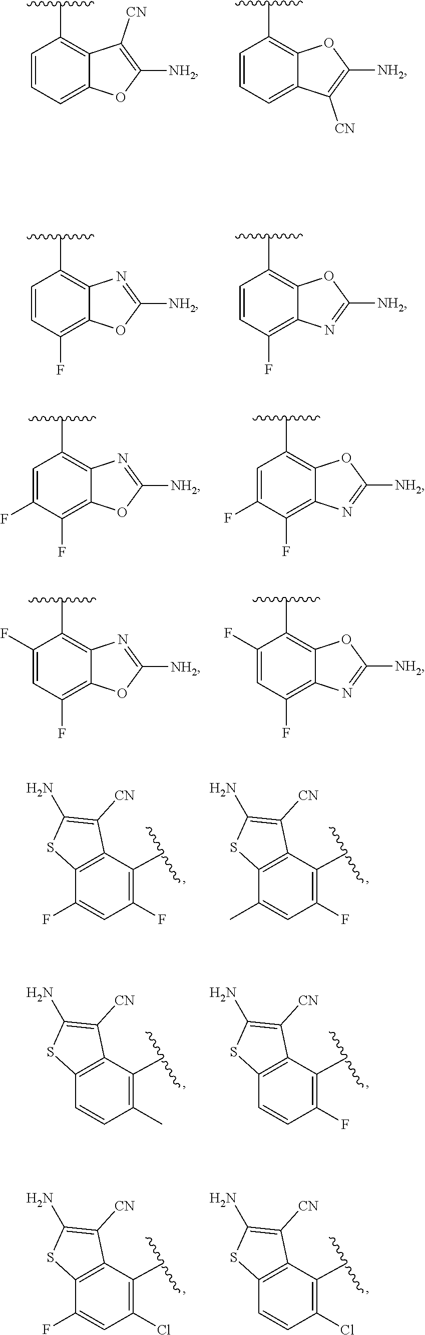

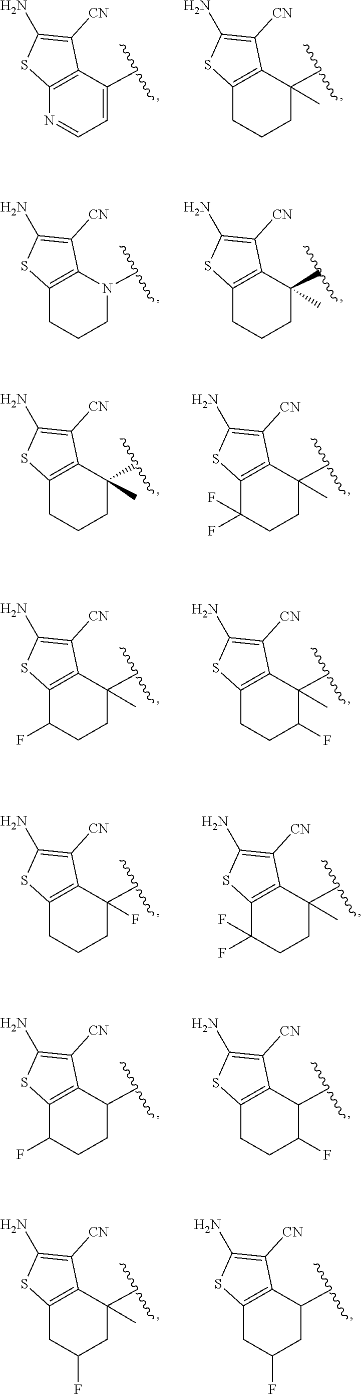

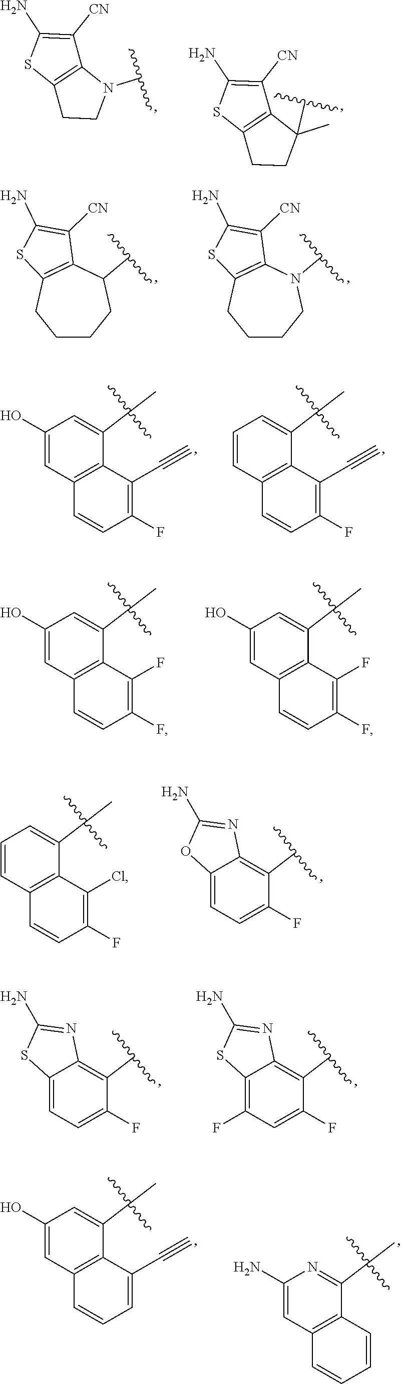

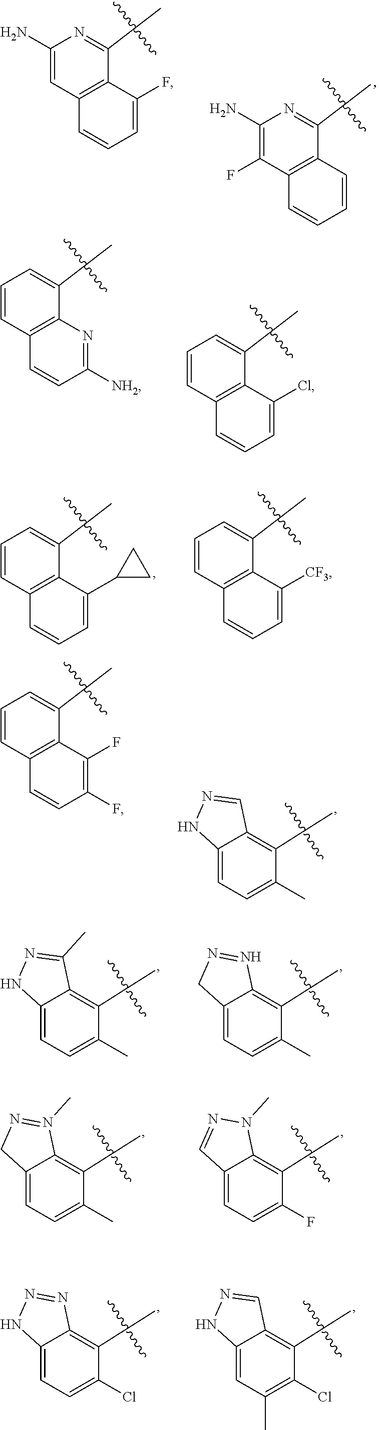

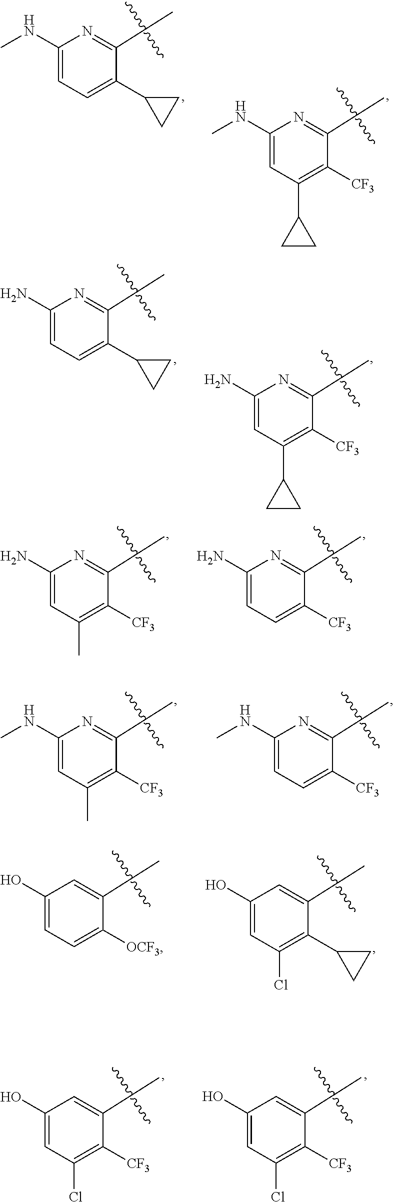

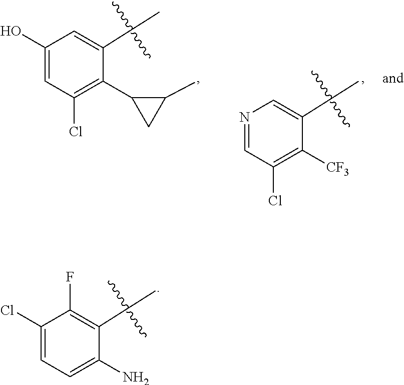 [SMILES: *c1cc(O)cc(Cl)c1C1CC1C ; *c1cncc(Cl)c1C(F)(F)F ; *c1c(N)ccc(Cl)c1F]

In embodiments, R¹⁹ is selected from

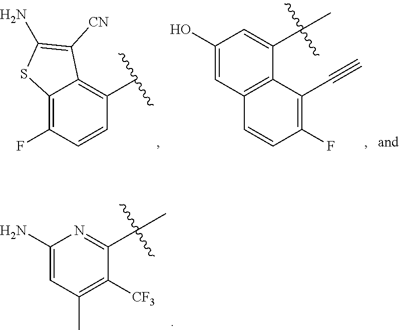 [SMILES: *c1ccc(F)c2sc(N)c(C#N)c12 ; *c1cc(O)cc2ccc(F)c(C#C)c12 ; *c1nc(N)cc(C)c1C(F)(F)F]

In embodiments, R¹⁹ is selected from

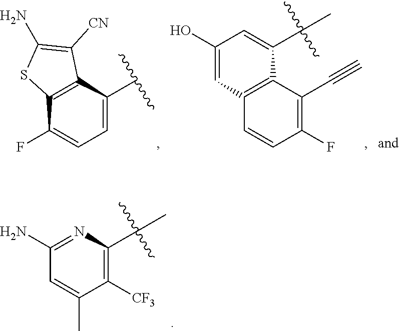

In embodiments, R is selected from

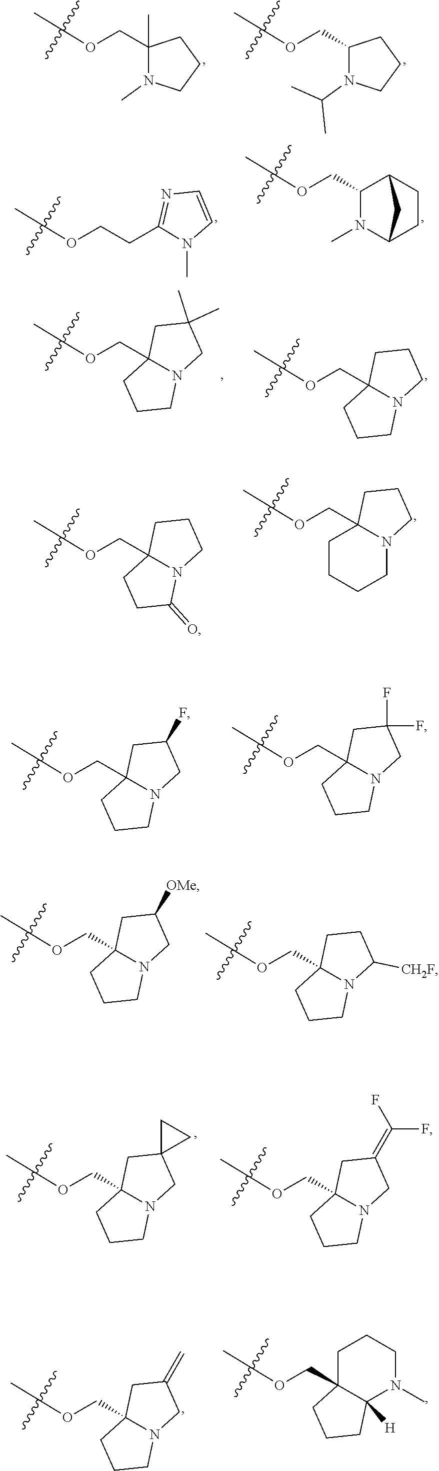

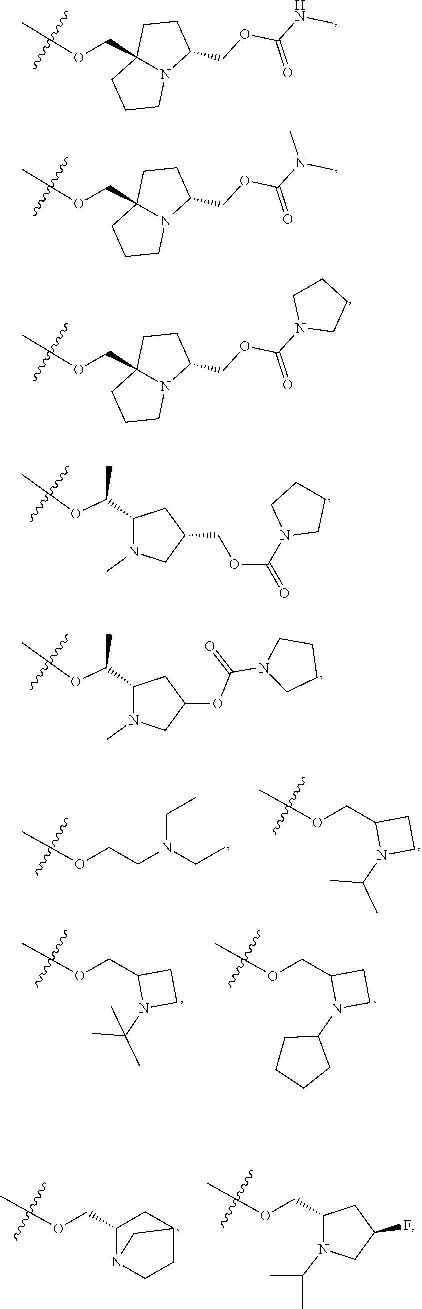

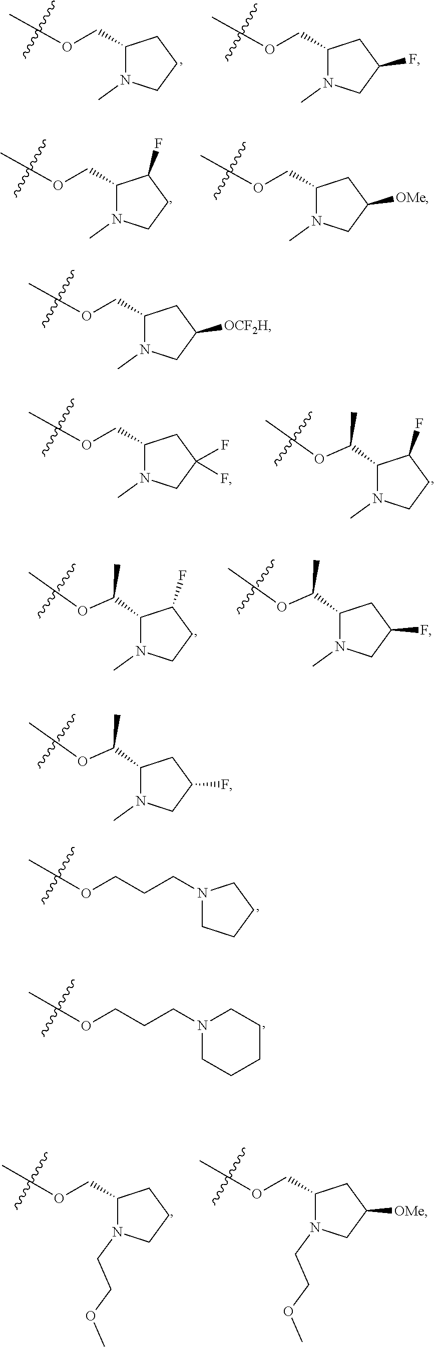

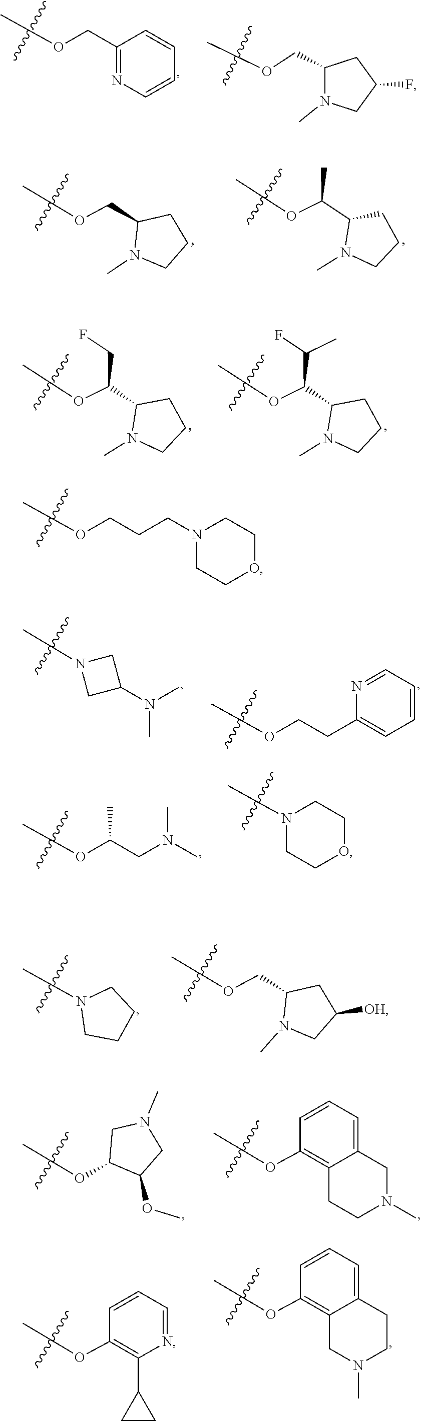

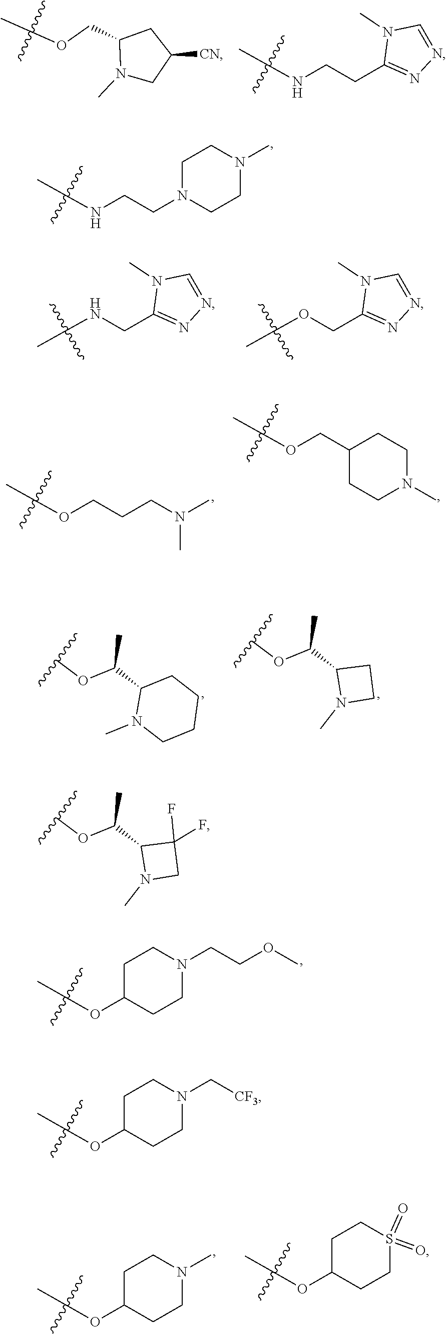

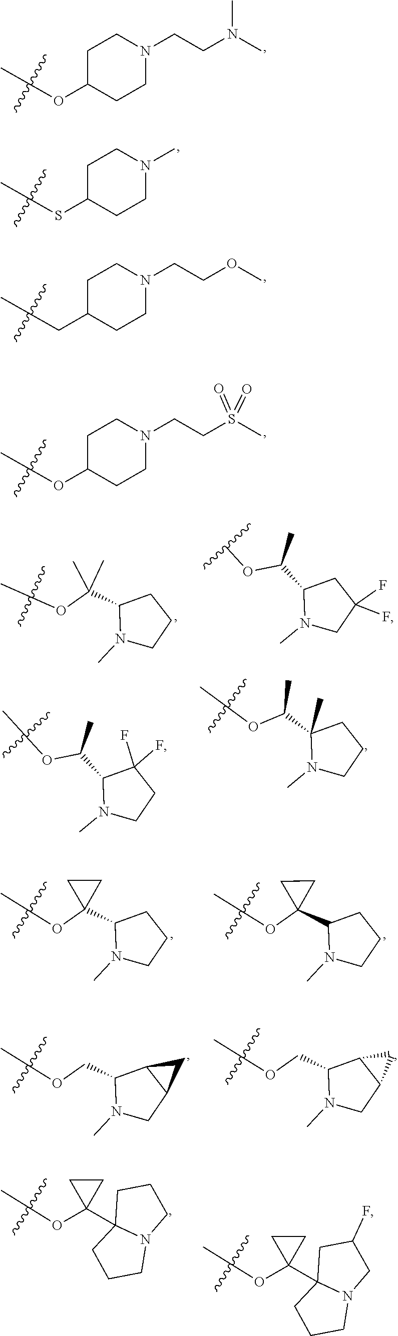

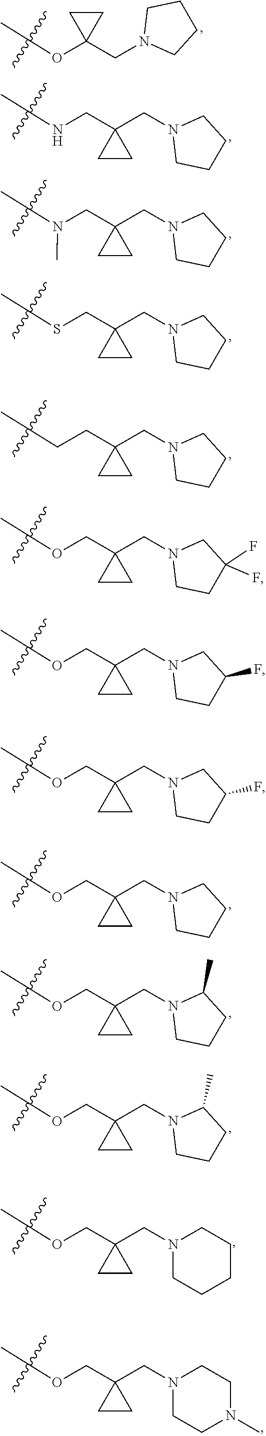

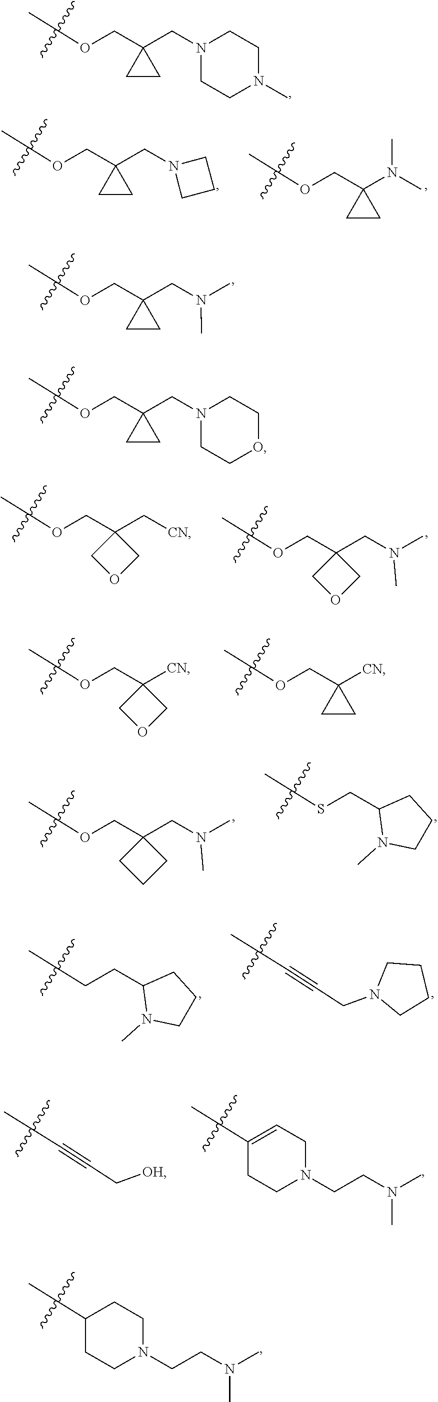

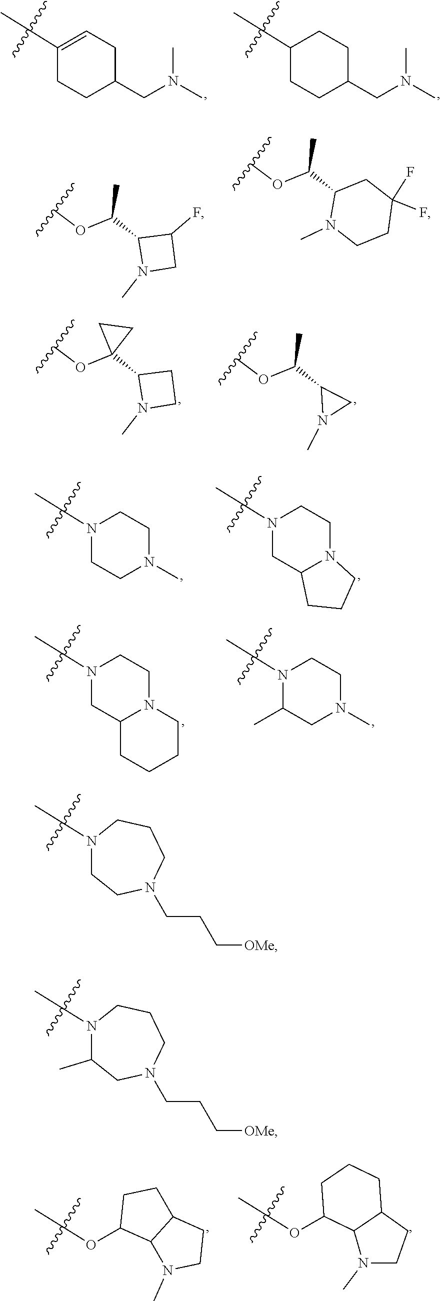

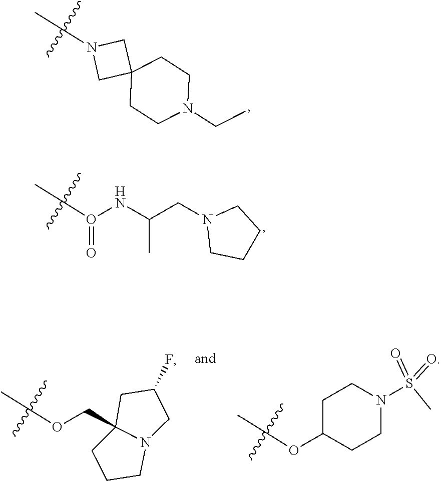

In embodiments, R² is selected from

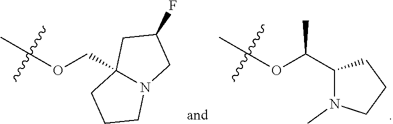

In an aspect is provided a pharmaceutical composition comprising a compound described herein, or a pharmaceutically acceptable salt or solvate thereof, and a pharmaceutically acceptable excipient.

In an aspect is provided a method of treating cancer in a subject in need thereof, comprising administering to the subject a therapeutically effective amount of a compound described herein, or a pharmaceutically acceptable salt or solvate thereof.

In embodiments, the cancer is a solid tumor or a hematological cancer.

In an aspect is provided a method of inhibiting cell growth, comprising administering an effective amount of a compound described herein, or a pharmaceutically acceptable salt or solvate thereof, to a cell expressing a Ras protein, thereby inhibiting growth of said cells.

In embodiments, the method comprises administering an additional agent.

In embodiments, additional agent comprises (1) an inhibitor of MEK; (2) an inhibitor of epidermal growth factor receptor (EGFR) and/or of mutants thereof; (3) an immunotherapeutic agent; (4) a taxane; (5) an anti-metabolite; (6) an inhibitor of FGFR1 and/or FGFR2 and/or FGFR3 and/or of mutants thereof; (7) a mitotic kinase inhibitor; (8) an anti-angiogenic drug; (9) a topoisomerase inhibitor; (10) a platinum-containing compound; (12) an inhibitor of c-MET and/or of mutants thereof; (13) an inhibitor of BCR-ABL and/or of mutants thereof; (14) an inhibitor of ErbB2 (Her2) and/or of mutants thereof; (15) an inhibitor of AXL and/or of mutants thereof; (16) an inhibitor of NTRK1 and/or of mutants thereof; (17) an inhibitor of RET and/or of mutants thereof; (18) an inhibitor of A-Raf and/or B-Raf and/or C-Raf and/or of mutants thereof; (19) an inhibitor of ERK and/or of mutants thereof; (20) an MDM2 inhibitor; (21) an inhibitor of mTOR20i; (23) an inhibitor of IGF1/2 and/or of IGF1-R; (24) an inhibitor of CDK9; (25) an inhibitor of farnesyl transferase; (26) an inhibitor of SHIP pathway; (27) an inhibitor of SRC; (28) an inhibitor of JAK; (29) a PARP inhibitor, (31) a ROS1 inhibitor; (32) an inhibitor of SHP pathway, or (33) an inhibitor of Src, FLT3, HDAC, VEGFR, PDGFR, LCK, Bcr-Abl or AKT; (34) an inhibitor of KrasG12C mutant; (35) a SHC inhibitor (e.g., PP2, AID371185); (36) a GAB inhibitor; (38) a PI-3 kinase inhibitor; (39) a MARPK inhibitor; (40) CDK4/6 inhibitor; (41) MAPK inhibitor; (42) SHP2 inhibitor; (43) checkpoint immune blockade agents; (44) or SOS1 inhibitor; or (45) a SOS 2 inhibitor.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a sequence alignment of various wild type Ras proteins including K-Ras, H-Ras, N-Ras, RalA, RalB, from top to bottom.

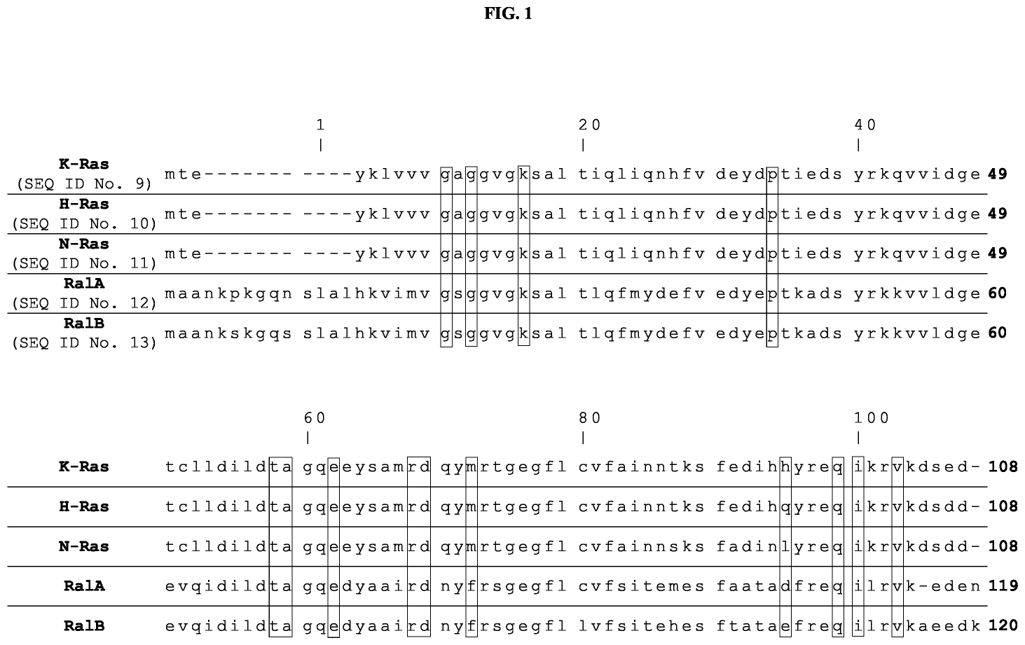

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4& Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6^{th} Edition (R. I. Freshney, ed. (2010)).

Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4^{th} Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to a straight or branched hydrocarbon chain radical consisting solely of carbon and hydrogen atoms, containing no unsaturation. In some embodiments, the “alkyl” group may have 1 to 18, 1 to 12, 1 to 10, 1 to 8, or 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆alkyl” or similar designations. By way of example only, “C₁-C₆alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, and hexyl. Alkyl groups can be substituted or unsubstituted. Depending on the structure, an alkyl group can be a monoradical or a diradical (i.e., an alkylene group).

An “alkoxy” refers to a “—O-alkyl” group, where alkyl is as defined herein.

The term “alkenyl” refers to a straight or branched hydrocarbon chain radical group consisting solely of carbon and hydrogen atoms, containing at least one carbon-carbon double bond. Non-limiting examples of an alkenyl group include —CH═CH₂, —C(CH₃)═CH₂, —CH═CHCH₃, —CH═C(CH₃)₂ and —C(CH₃)═CHCH₃. In some embodiments, an alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a straight or branched hydrocarbon chain radical group consisting solely of carbon and hydrogen atoms, containing at least one carbon-carbon triple bond. Non-limiting examples of an alkynyl group include —C≡CH, —C≡CCH₃, —C≡CCH₂CH₃ and —C≡CCH₂CH₂CH₃. In some embodiments, an alkynyl group can have 2 to 6 carbons. Alkynyl groups can be substituted or unsubstituted. Depending on the structure, an alkynyl group can be a monoradical or a diradical (i.e., an alkynylene group).

“Amino” refers to a —NH₂ group.

The term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂ group, where alkyl is as defined herein.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to a monocyclic aromatic ring wherein each of the atoms forming the ring is a carbon atom (e.g., phenyl) or a polycyclic ring system (e.g., bicyclic or tricyclic) wherein 1) at least one ring is carbocyclic and aromatic, 2) a bond to the remainder of the compound is directly bonded to a carbocyclic aromatic ring of the aryl ring system, and 3) the carbocyclic aromatic ring of the aryl ring system of 2) is not directly bonded (e.g., fused) to a heteroaryl ring in the polycyclic ring system. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group). As used herein, the aryl radical is a monocyclic, bicyclic, or tricyclic ring system. In embodiments, an aryl is a monocyclic ring. In embodiments, an aryl is a fused ring polycyclic system. In embodiments, an aryl is a bridged ring polycyclic system. In some embodiments the aryl is a “fused ring aryl” wherein the aryl ring is fused with a cycloalkyl or a heterocycloalkyl ring.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

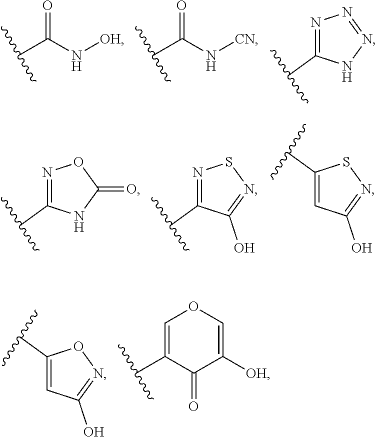

and the like.

The term “cycloalkyl” refers to a monocyclic carbocyclic saturated or partially unsaturated non-aromatic ring or a polycyclic carbocyclic (i.e., does not include heteroatom(s)) ring system (e.g., bicyclic or tricyclic) wherein 1) at least one ring is carbocyclic saturated or partially unsaturated and non-aromatic, 2) a bond to the remainder of the compound is directly bonded to a carbocyclic saturated or partially unsaturated non-aromatic ring of the ring system, and 3) the carbocyclic saturated or partially unsaturated non-aromatic ring of the ring system of 2) is not directly bonded (e.g., fused or spirocyclic) to a heterocycloalkyl ring in the polycyclic ring system. Cycloalkyls may be saturated or partially unsaturated. In some embodiments, a cycloalkyl ring is a spirocyclic cycloalkyl ring. In embodiments, a cycloalkyl is a monocyclic ring. In embodiments, a cycloalkyl is a fused ring polycyclic system. In embodiments, a cycloalkyl is a bridged ring polycyclic system. In embodiments, a cycloalkyl is a spirocyclic polycyclic ring system. In some embodiments, cycloalkyl groups include groups having from 3 to 10 ring atoms. Depending on the structure, a cycloalkyl group can be a monoradical or a diradical (i.e., a cycloalkylene group).

The terms “heteroaryl” or, alternatively, “heteroaromatic” refers to an monocyclic aryl group that includes one or more ring heteroatoms selected from nitrogen, oxygen and sulfur; or a polycyclic ring system (e.g., bicyclic or tricyclic) wherein 1) at least one ring is aromatic and includes one or more heteroatoms selected from nitrogen, oxygen and sulfur and 2) a bond to the remainder of the compound is directly bonded to an aromatic ring including one or more heteroatoms selected from nitrogen, oxygen and sulfur or an aromatic ring directly bonded (e.g., fused) to an aromatic ring including one or more heteroatoms selected from nitrogen, oxygen and sulfur, of the aryl ring system. As used herein, the heteroaryl radical may be a monocyclic, bicyclic, or tricyclic ring system, wherein at least one of the rings in the ring system is fully unsaturated (i.e., aromatic) and includes a heteroatom. In embodiments, a heteroaryl is a monocyclic ring. In embodiments, a heteroaryl is a fused ring polycyclic system. In embodiments, a heteroaryl is a bridged ring polycyclic system. In some embodiments is a “fused ring heteroaryl” wherein the heteroaryl ring is fused with a cycloalkyl, aryl, or heterocycloalkyl ring. An N-containing “heteroaromatic” or “heteroaryl” moiety refers to an aromatic group in which at least one of the skeletal atoms of the ring is a nitrogen atom. Depending on the structure, a heteroaryl group can be a monoradical or a diradical (i.e., a heteroarylene group).

A “heterocycloalkyl” group or “heteroalicyclic” group refers to a cycloalkyl group, wherein at least one skeletal ring atom of a saturated or partially unsaturated non-aromatic ring is a heteroatom selected from nitrogen, oxygen, phosphorus, and sulfur. A heterocycloalkyl refers to a monocyclic saturated or partially unsaturated non-aromatic ring including one or more heteroatoms or a polycyclic ring system (e.g., bicyclic or tricyclic) wherein 1) at least one ring is saturated or partially unsaturated, non-aromatic, and includes one or more heteroatoms and 2) a bond to the remainder of the compound is directly bonded to a ring of the ring system that is a saturated or partially unsaturated and non-aromatic ring that includes one or more heteroatoms or a non-aromatic ring directly bonded (e.g., fused) to a saturated or partially unsaturated and non-aromatic ring that includes one or more heteroatoms of the ring system. Heterocycloalkyls may be saturated or partially unsaturated. The term heterocycloalkyl also includes all ring forms of the carbohydrates, including but not limited to the monosaccharides, the disaccharides and the oligosaccharides. In some embodiments, a heterocycloalkyl ring is a spirocyclic heterocycloalkyl ring. In embodiments, a heterocycloalkyl is a monocyclic ring. In embodiments, a heterocycloalkyl is a fused ring polycyclic system. In embodiments, a heterocycloalkyl is a bridged ring polycyclic system. In embodiments, a heterocycloalkyl is a spirocyclic polycyclic ring system. Unless otherwise noted, heterocycloalkyls have from 2 to 13 carbons in the ring or ring system. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Depending on the structure, a heterocycloalkyl group can be a monoradical or a diradical (i.e., a heterocycloalkylene group).

The term “halo” or, alternatively, “halogen” means fluoro, chloro, bromo and iodo.

The abbreviations “Fmoc”, “Ac”, “Bn”, “PMB”, “Tr”, “Ts”, “Boc”, and “Cbz” are used in accordance with their well understood common meanings in Chemistry and mean the monovalent chemical substituents fluorenylmethyloxycarbonyl, acetyl, benzyl, p-methoxybenzyl, trityl or triphenylnethyl, tosyl, tert-butyloxycarbonyl, and carbobenzyloxy, respectively.

The term “haloalkyl” refers to an alkyl group that is substituted with one or more halogens. The halogens may the same or they may be different. Non-limiting examples of haloalkyls include —CH₂Cl, —CF₃, —CHF₂, —CH₂CF₃, —CF₂CF₃, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “oxo” refers to the ═O radical.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

The suffix “-di-yl” will be understood to mean the substituent or linker is a divalent substituent or linker.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

“Optional” or “optionally” means that a subsequently described event or circumstance may or may not occur and that the description includes instances when the event or circumstance occurs and instances in which it does not.

The term “optionally substituted” or “substituted” means, unless otherwise specified, that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L^{s}R^{s}, wherein each L^{s} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R^{s} is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), Morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “agent” or “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (also used interchangeably herein as K-Ras, K-ras, Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof. The terms “mutant Ras” and “Ras mutant,” as used interchangeably herein, refer to a Ras protein with one or more amino acid mutations, such as with respect to a common reference sequence such as a wild-type (WT) sequence. In some embodiments, a mutant Ras is selected from a mutant KRAS, mutant HRAS, mutant NRAS, mutant MRAS, mutant ERAS, mutant RRAS2, mutant RALA, mutant RALB, mutant RIT1, and any combination thereof, such as from a mutant KRAS, mutant HRAS, mutant NRAS, mutant RALA, mutant RALB, and any combination thereof. In some embodiments, a mutation can be an introduced mutation, a naturally occurring mutation, or a non-naturally occurring mutation. In some embodiments, a mutation can be a substitution (e.g., a substituted amino acid), insertion (e.g., addition of one or more amino acids), or deletion (e.g., removal of one or more amino acids). In some embodiments, two or more mutations can be consecutive, non-consecutive, or a combination thereof. In some embodiments, a mutation can be present at any position of Ras. In some embodiments, a mutation can be present at position 12, 13, 62, 92, 95, or any combination thereof of Ras of SEQ ID No. 2 when optimally aligned. In some embodiments, a mutant Ras may comprise about or at least about 1, 2, 3, 4, 5, 6, 7, 8, 9, 10, 11, 12, 13, 14, 15, 16, 17, 18, 19, 20, 25, 30, 35, 40, 45, 50, or more than 50 mutations. In some embodiments, a mutant Ras may comprise up to about 1, 2, 3, 4, 5, 6, 7, 8, 9, 10, 11, 12, 13, 14, 15, 16, 17, 18, 19, 20, 25, 30, 35, 40, 45, or 50 mutations. In some embodiments, the mutant Ras is about or up to about 500, 400, 300, 250, 240, 233, 230, 220, 219, 210, 208, 206, 204, 200, 195, 190, 189, 188, 187, 186, 185, 180, 175, 174, 173, 172, 171, 170, 169, 168, 167, 166, 165, 160, 155, 150, 125, 100, 90, 80, 70, 60, 50, or fewer than 50 amino acids in length. In some embodiments, an amino acid of a mutation is a proteinogenic, natural, standard, non-standard, non-canonical, essential, non-essential, or non-natural amino acid. In some embodiments, an amino acid of a mutation has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, a mutation comprises a reactive moiety. In some embodiments, a substituted amino acid comprises a reactive moiety. In some embodiments, a mutant Ras can be further modified, such as by conjugation with a detectable label. In some embodiments, a mutant Ras is a full-length or truncated polypeptide. For example, a mutant Ras can be a truncated polypeptide comprising residues 1-169 or residues 11-183 (e.g., residues 11-183 of a mutant RALA or mutant RALB).

As used herein, the term “corresponding to” or “corresponds to” as applied to an amino acid residue in a polypeptide sequence refers to the correspondence of such amino acid relative to a reference sequence when optimally aligned (e.g., taking into consideration of gaps, insertions and mismatches). For instance, the serine residue in a Ras G12S mutant refers to the serine corresponding to residue 12 of SEQ ID No. 1, which can serves as a reference sequence. A modified Ras mutant protein disclosed herein may comprise truncations at C-terminus, or truncations at the N-terminal end preceding the serine residue. The serine residue in such N-terminal truncated modified mutant is still considered corresponding to position 12 of SEQ ID No. 1. In addition, serine residue at position 12 of SEQ ID No. 1 finds a corresponding residue in SEQ ID Nos. 3 and 5. “Prodrug” as used herein is meant to indicate a compound that may be converted under physiological conditions or by solvolysis to a biologically active compound described herein. The term “prodrug” refers to a precursor of a biologically active compound that is pharmaceutically acceptable. A prodrug may be inactive when administered to a subject, but is converted in vivo to an active compound, for example, by hydrolysis. The prodrug compound may offer advantages of solubility, tissue compatibility and/or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. A “prodrug” can be any covalently bonded carriers, that release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound.

The terms “leaving group” is used herein in accordance with their well understood meanings in Chemistry and refers to an atom or group of atoms which breaks away from the rest of the molecule, taking with it the electron pair which used to be the bond between the leaving group and the rest of the molecule.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In embodiments, the degradation enhancer is capable of binding to an E3 ubiquitin ligase protein or a protein complex comprising an E3 ubiquitin ligase protein. In embodiments, the degradation enhancer is capable of binding to an E2 ubiquitin-conjugating enzyme. In embodiments, the degradation enhancer is capable of binding to a protein complex comprising an E2 ubiquitin-conjugating enzyme and an E3 ubiquitin ligase protein.

[symbol] indicates the location of attachment (e.g., location of a bond to another atom) of the depicted chemical formula or atom to a substituent, a further component of a molecule, or an atom. [symbol] may equivalently be located at the end of a bond or overlapping a bond. A waved line [symbol] drawn across a bond or at the end of a bond and a dashed bond “ [symbol] ” are used interchangeably herein to denote where a bond disconnection or attachment occurs.

[symbol]

### Compounds

In an aspect is provided a compound of Formula (I), or a pharmaceutically acceptable salt or solvate thereof:

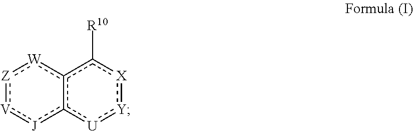

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16b}); wherein exactly one of V and J is C(R¹⁷), C(R¹⁷)(R^{16a}), or N(R¹⁷);
Y is N, C(R²), C(R²)(R¹), N(R^{2b}), S(O), or S(O)₂;
U is N, C(R¹), C(R¹)(R¹), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is

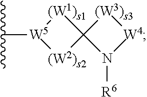

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵, wherein R⁶ is optionally capable of forming a covalent bond with a Ras amino acid;
L² is a bond, —O—, —N(R^{4d})—, —C(O)—, —S—, —S(O)₂—, —S(O)—, —P(O)R^{4d}—, CR^{4c}R^{4c}, —OCR^{4c}R^{4c}—, —N(R^{4d})CR^{4c}R^{4c}—, —C(O)CR^{4c}R^{4c}—, —SCR^{4c}R^{4c}—, —S(O)₂CR^{4c}R^{4c}—, —S(O)CR^{4c}R^{4c}—, —P(O)R^{4d}CR^{4c}R^{4c}—, —CR^{4c}R^{4c}R^{4c}R^{4c}, —CR^{4c}R^{4c}O—, —CR^{4c}R^{4c}N(R^{4d})—, —CR^{4c}R^{4c}C(O)—, —CR^{4c}R^{4c}S—, —CR^{4c}R^{4c}S(O)₂—, —CR^{4c}R^{4c}S(O)—, —CR^{4c}R^{4c}P(O)R^{4d}—, —N(R^{4d})C(O)—, —N(R^{4d})S(O)₂—, —N(R^{4d})S(O)—, —N(R^{4d})P(O)R^{4d}—, —C(O)N(R^{4d})—, —S(O)₂N(R^{4d})—, —S(O)N(R^{4d})—, —P(O)R^{4d}N(R^{4d})—, —OC(O)—, —OS(O)₂—, —OS(O)—, —OP(O)R^{4d}—, —C(O)O—, —S(O)₂O—, —S(O)O—, —P(O)R^{4d}O—, —CR^{4c}R^{4c}R^{4c}R^{4c}CR^{4c}R^{4c}—, —OCR^{4c}R^{4c}CR^{4c}R^{4c}—, —N(R^{4d})CR^{4c}R^{4c}R^{4c}R^{4c}, —C(O)CR^{4c}R^{4c}CR^{4c}R^{4c}—, —SCR^{4c}R^{4c}R^{4c}R^{4c}—, —S(O)₂CR^{4c}R^{4c}CR^{4c}R^{4c}—, —S(O)CR^{4c}R^{4c}CR^{4c}R^{4c}—, —P(O)R^{4d}CR^{4c}R^{4c}R^{4c}R^{4c}—, —CR^{4c}R^{4c}R^{4c}R^{4c}O—, —CR^{4c}R^{4c}CR^{4c}R^{4c}N(R^{4d})—, —CR^{4c}R^{4c}R^{4c}R^{4c}C(O)—, —CR^{4c}R^{4c}CR^{4c}R^{4c}S—, —CR^{4c}R^{4c}R^{4c}R^{4c}S(O)₂—, —CR^{4c}R^{4c}R^{4c}R^{4c}S(O)—, or —CR^{4c}R^{4c}R^{4c}P(O)R^{20a};
each R^{4c} is independently selected from hydrogen, halogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, —OC(O)R^{14a}, —N(R¹⁴)(R¹⁴), —N(R¹⁴)C(O)N(R¹⁴)(R¹⁴), —N(R¹⁴)C(O)OR¹⁴, —N(R¹⁴)C(O)R^{14a}, and —N(R¹⁴)S(O)₂R¹⁴, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
each R¹ is independently selected from hydrogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, and —OC(O)R^{14a}, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_g S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²¹;
R¹⁷ is -L¹-R¹⁹;
R^{17b} is -L^{1b}-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R¹), S(O)₂N(R^{1c}), S(O)N(R^{1c}), C(R^{1f})(R^{1g})O, C(R^{1f})(R^{1a})N(R^{1c}), and C(R¹)(R^{g}); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R¹⁹ are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2c} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR^{12′}, —SR^{12′}, —C(O)OR^{12′}, —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{2d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R²;
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R²⁰, R^{20d}, R^{20e}, R^{20f}, R^{20g}, R^{20h}, R²⁰ⁱ, R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
[symbol] indicates the location of attachment of the depicted chemical formula or atom to a substituent, a further component of molecule, or an atom; and
[symbol] indicates a single or double bond such that all valences are satisfied.

In an aspect is provided a compound of Formula (II), or a pharmaceutically acceptable salt or solvate thereof:

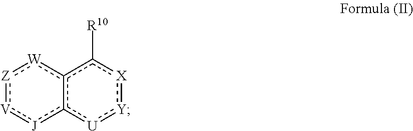

wherein
W, Z, V, J, U, R¹⁰, L⁷, W¹, W³, W², W⁴, W⁵, s1, s2, s3, R¹, R⁴, R⁶, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R^{16b}, R², R^{2b}, R^{12b}, X, R³, R¹², R^{12′}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵ are as described for Formula (I), including in embodiments of a compound of Formula (I);
Y is N, C(R²), C(R²)(R^{2c}), N(R^{2b}), S(O), S(O)₂, or C(O);
R⁷ is

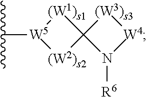

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, three, four, five, six, or seven R¹ⁱ;
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d}; and
[symbol] indicates a single or double bond such that all valences are satisfied.

In an aspect is provided a compound of Formula (IV), or a pharmaceutically acceptable salt or solvate thereof:

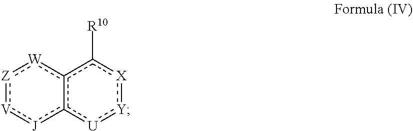

wherein
W, Z, V, J, U, R¹⁰, L⁷, W¹, W³, W², W⁴, W⁵, s3, R¹, R⁴, R⁸, R^{8a}, R^{8b}, R¹⁷, R^{17b}, L¹, L^{1b}, R^{1e}, R^{1f}, R^{1g}, R^{1c}, R¹⁹, R¹ⁱ, R¹⁶, R^{16a}, R¹⁶, R², R^{2c}, R^{2b}, R^{12b}, X, R³, R¹², R^{12′}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20m}, R²¹, R²², R²³, R²⁴, and R²⁵ are as described for Formula (I), including in embodiments of a compound of Formula (I);
Y is N, C(R²), C(R²)(R^{2c}), N(R^{2b}), S(O), S(O)₂, or C(O);
R⁷ is

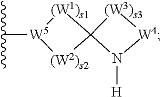

s1 is an integer from 1 to 6 and s2 is an integer from 2 to 3; or s1 is an integer from 2 to 6 and s2 is an integer from 1 to 3;
each R^{12″} is independently selected from C₃₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₃₋₆alkyl, C₂₋₆alkenyl, C₂₋₆alkynyl, C₃₋₁₀cycloalkyl, —C(R¹²)₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d}; and [symbol] indicates a single or double bond such that all valences are satisfied.

In an aspect is provided a compound of Formula (I′), or a pharmaceutically acceptable salt or solvate thereof:

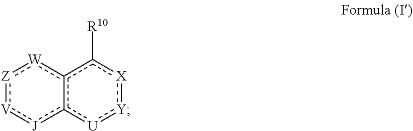

wherein
W, Z, V, J, Y, U, R¹⁰, L⁷, R⁷, W¹, W³, W², W⁴, W¹, s1, s2, s3, R¹, R⁶, L², R^{4c}, R^{4d}, R⁸, R^{8a}, R^{8b}, R¹⁷, R¹⁷, L¹, L^{1b}, R^{1e}, R¹, R^{1g}, R^{1e}, R¹⁹, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R²¹, R²², R²³, R²⁴, and R²⁵ are as described for Formula (I), including in embodiments of a compound of Formula (I);
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), ═C(R^{21b})₂, —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH—)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl), —S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl)-S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k};
R² is selected from halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₄₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₄₋₉heterocycloalkyl, C₆₋₁₀aryl, and C₁₋₉heteroaryl are optionally substituted with one, two, or three R^{20d};
each R^{20a}, R^{20b}, R²⁰, R^{20d}, R^{20e}, R^{20f}, R^{20g}, R^{20h}, R²⁰ⁱ, R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), ═C(R^{21b})₂, —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²¹, —N(R²⁴)S(O)₂R²⁵, —C(O)R²¹, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
[symbol] indicates the location of attachment of the depicted chemical formula or atom to a substituent, a further component of a molecule, or an atom;
R^{21b} is independently selected at each occurrence from hydrogen, halogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, or two R^{21b} are taken together with the carbon atom to which they are attached to form C₃₋₁₀cycloalkyl or C₂₋₉heterocycloalkyl; each of which is optionally substituted with one, two, or three substituents independently selected from halogen, C₁₋₃ alkyl, C₁₋₃ haloalkyl, and —OH; and
[symbol] indicates a single or double bond such that all valences are satisfied.

In an aspect is provided a compound of Formula (II′), or a pharmaceutically acceptable salt or solvate thereof:

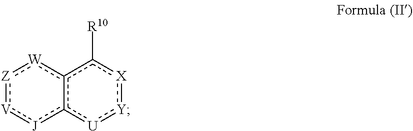

wherein
W, Z, V, J, U, L⁷, W¹, W³, W², W⁴, W⁵, s1, s2, s3, R¹, R⁴, R⁶, R⁸, R^{8a}, R^{8b}, R¹⁷, R^{17b}, L¹, L^{1b}, R^{1e}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
Y is N, C(R²), C(R²)(R^{2c}), N(R^{2b}), S(O), S(O)₂, or C(O);
R¹⁰ is -L⁷-R⁷;
R⁷ is

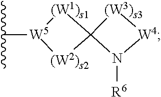

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R¹⁹ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl), —S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R¹ⁱ;
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates the location of attachment of the depicted chemical formula or atom to a substituent, a further component of a molecule, or an atom; and
[symbol] indicates a single or double bond such that all valences are satisfied.

In an aspect is provided a compound of Formula (II″), or a pharmaceutically acceptable salt or solvate thereof:

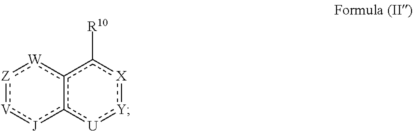

wherein
W, Z, V, J, U, L⁷, W¹, W³, W², W⁴, W⁵, s1 s2, s3, R, R⁴, R⁶, R⁸, R^{8a}, R^{8b}, R¹⁷, R¹⁷, L¹, L^{1b}, R^{1e}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
Y is N, C(R²), C(R²)(R^{2c}), N(R^{2b}), S(O), S(O)₂, or C(O);
R¹⁰ is -L⁷-R⁷;
R⁷ is

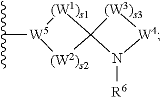

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁ heterocycloalkyl, C₆₋₁₂aryl, and C₁₋₁₁heteroaryl are optionally substituted with one, two, or three R^{20k};
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl), —S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R¹ⁱ;
R² is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates the location of attachment of the depicted chemical formula or atom to a substituent, a further component of a molecule, or an atom; and
[symbol] indicates a single or double bond such that all valences are satisfied.

In an aspect is provided a compound of Formula (IV′), or a pharmaceutically acceptable salt or solvate thereof:

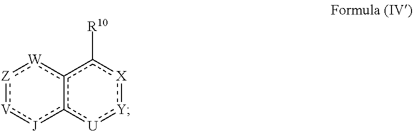

wherein
W, Z, V, J, U, L⁷, W³, W², W⁵, s3, R¹, R⁴, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{1a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
Y is N, C(R²), C(R²)(R^{2c}), N(R^{2b}), S(O), S(O)₂, or C(O);
R¹⁰ is -L⁷-R⁷;
R⁷ is

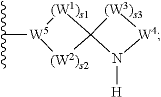

W⁴ is selected from C(R¹)(R¹), C(R¹)(R⁴), or C(R⁴)(R⁴);
s1 is an integer from 1 to 6 and s2 is an integer selected from 2 and 3; or s1 is an integer from 2 to 6 and s2 is an integer from 1 to 3;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
R² is hydrogen, halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉heteroaryl, —OR^{12″}, —SR^{12′}, —N(R^{12″″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₄₋₉ heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
each R^{12″} is independently selected from hydrogen, C₂₋₆alkyl, C₂₋₆alkenyl, C₂₋₆alkynyl, C₃₋₁₀cycloalkyl, —C(R^{12c})₂—C₄₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₄₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d}; and
[symbol] indicates a single or double bond such that all valences are satisfied.

In an aspect is provided a compound of Formula (IV′), or a pharmaceutically acceptable salt or solvate thereof:

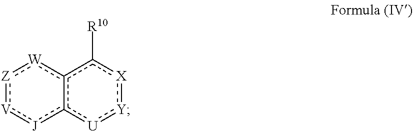

wherein
W, Z, V, J, U, L⁷, W³, W², W¹, s3, R¹, R⁴, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
Y is N, C(R²), C(R²)(R^{2c}), N(R^{2b}), S(O), S(O)₂, or C(O);
R¹⁰ is -L⁷-R⁷;
R⁷ is

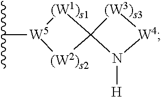

W⁴ is selected from C(R¹)(R¹), C(R¹)(R⁴), or C(R⁴)(R⁴);
s1 is an integer from 1 to 6 and s2 is an integer selected from 2 and 3; or s1 is an integer from 2 to 6 and s2 is an integer from 1 to 3;
s1, s2, and s3 are not all 2;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
R² is hydrogen, halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉heteroaryl, —OR^{12″}, —SR^{12′}, —N(R^{12″″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R⁹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₄₋₉ heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
each R^{12″} is independently selected from hydrogen, C₂₋₆alkyl, C₂₋₆alkenyl, C₂₋₆alkynyl, C₃₋₁₀cycloalkyl, —C(R^{12c})₂—C₄₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₄₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d}; and
[symbol] indicates a single or double bond such that all valences are satisfied.

In an aspect is provided a compound of Formula (Ia), or a pharmaceutically acceptable salt or solvate thereof:

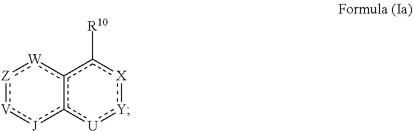

wherein
W, Z, V, J, U, R¹⁰, L⁷, R⁷, W, W³, W², W¹, s1, s2, s3, R¹, R⁴, R⁶, L², R⁵, R^{4c}, R^{4d}, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
Y is N, C(R²), C(R²)(R^{2c}), N(R^{2b}), S(O), S(O)₂, or C(O);
W⁴ is selected from C(R¹)(R¹), C(R¹)(R⁴), and C(R⁴)(R⁴);
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R¹⁹ is selected from C₂₋₁₁heterocycloalkyl and C₂₋₁₂heteroaryl, wherein the C₂₋₁₁heterocycloalkyl and C₂₋₁₂heteroaryl are optionally substituted with one, two, three, four, five, six, or seven R¹ⁱ; and
[symbol] indicates a single or double bond such that all valences are satisfied.

In an aspect is provided a compound of Formula (Ib), or a pharmaceutically acceptable salt or solvate thereof:

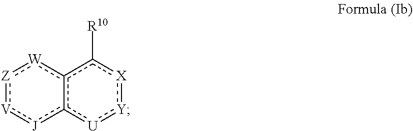

wherein
W, Z, V, J, U, R¹⁰, L⁷, R⁷, W¹, W³, W², W⁴, W⁵, s1, s2, s3, R¹, R⁴, R⁶, L², R⁵, R^{1c}, R^{4d}, R⁸, R^{8a}, R^{8b}, R¹⁷, R^{17b}, L₁, L^{1b}, R^{1e}, R^{1f}, R^{1g}, R^{1c}, R¹ⁱ, R¹⁶, R¹⁶, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20s}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
Y is N, C(R²), C(R²)(R^{2c}), N(R^{2b}), S(O), S(O)₂, or C(O);
R² is hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C_{2- 9}heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};

R¹⁹ is;

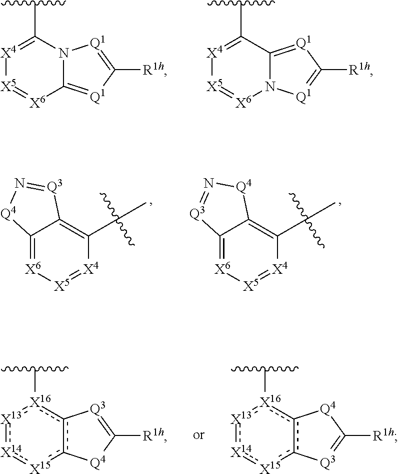

Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, X¹⁸ are independently selected from a C(O), C(R^{1a}), N, C(R^{1a})(R^{1b}), and N(R^{1c}); X¹⁶ are independently selected from C, N, and C(R^{1a});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ;
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; and
[symbol] indicates a single or double bond such that all valences are satisfied.

In an aspect is provided a compound of Formula (IVa), or a pharmaceutically acceptable salt or solvate thereof:

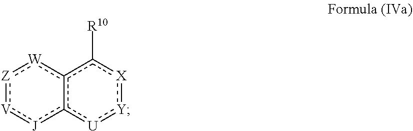

wherein
   W, Z, V, J, U, Y, L⁷, R⁷, W¹, W³, W², W¹, R⁴, R⁵, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (IV′), including in embodiments of a compound of Formula (IV′);
   R¹⁰ is -L⁷-R⁷;
   W⁴ is selected from C(R¹)(R¹), C(R¹)(R⁴), C(R⁴)(R⁴), C(O), S(O), and S(O)₂;
   s1 is an integer from 1 to 6;
   s2 is an integer from 1 to 3;
   s3 is an integer from 1 to 3;
   R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12″}, —SR^{12′}, —N(R^{12″″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
   each R^{12″} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
   each R^{12″″} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
   R¹⁹ is:

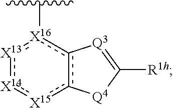

Q³ is C(R^{1d}); Q⁴ is S;
X¹³ is C(R^{1a}); X¹⁴ and X¹⁵ are independently C(R^{1a}); X¹⁶ is C;
each R^{1a}, R^{1d}, and R^{1h} is independently selected from hydrogen, halogen, —CN, C₁₋₆alkyl, C₂₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; and
[symbol] indicates a single or double bond such that all valences are satisfied.

A compound of Formula (IVa), or a pharmaceutically acceptable salt or solvate thereof:

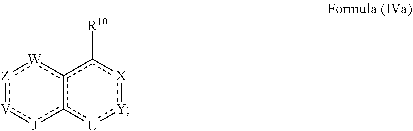

wherein
   W, Z, V, J, U, Y, L⁷, R⁷, W, W³, W², W⁵, R¹, R⁴, R⁸, R^{8a}, R^{8b}, R¹⁷, R¹⁷, L¹, L^{1b}, R^{1e}, R^{1f}, R^{1g}, R^{1c}, R¹ⁱ, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰, R^{20k}, R^{20m}, R²¹, R^{21b}, R²², R²³, R²⁴, and R²⁵ are as described for Formula (IV′), including in embodiments of a compound of Formula (IV′);
   R¹⁰ is -L⁷-R⁷;
   W⁴ is selected from C(R¹)(R¹), C(R¹)(R⁴), C(R⁴)(R⁴), C(O), S(O), and S(O)₂;
   s1 is an integer from 1 to 6;
   s2 is an integer from 1 to 3;
   s3 is an integer from 1 to 3;
   s1, s2, and s3 are not all 2;
   R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12″}, —SR^{12′}, —N(R^{12″″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═N)(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
   each R^{12″} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
   each R^{12″″} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R¹²)₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
   R¹⁹ is:

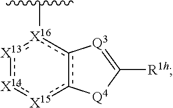

Q³ is C(R^{1d}); Q⁴ is S;
X¹³ is C(R^{1a}); X¹⁴ and X¹⁵ are independently C(R^{1a}); X¹⁶ is C;
each R^{1a}, R^{id}, and R^{1h} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₁₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₁₋₁₀aryl, and C₁₋₉heteroaryl are optionally substituted with one, two, or three R²⁰ⁱ; and
[symbol] indicates a single or double bond such that all valences are satisfied.

In an aspect is provided a compound of Formula (Ic), or a pharmaceutically acceptable salt or solvate thereof:

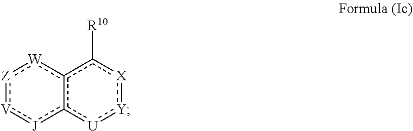

wherein
S1, s2, s3, R, R⁴, R⁶, R^{4c}, R^{4d}, R¹⁷, L^{1b}, R^{1e}, R^{1f}, R^{1g}, R^{1c}, R¹⁹, R¹ⁱ, R^{16a}, R¹⁶, R^{2c}, R^{2b}, R^{12b}, R³, R¹², R^{12′}, R^{12″}, R^{12c}, R¹³, R¹⁴, R^{14a}, R¹⁵, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
W is a C(R¹⁸) or C(O); Z is N, C(R⁸), or N(R^{1b}); V is C(R¹⁷); J is C(R¹⁶); Y is C(R²); U is N; X is N;
R¹⁰ is -L⁷-R⁷;
L⁷ is a bond;
R⁷ is

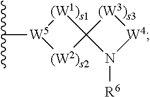

W¹, W², and W³ are independently selected from C(R¹)(R¹), C(R¹)(R⁴), C(R⁴)(R⁴), and O;
W⁴ is selected from C(R¹)(R¹), C(R¹)(R⁴), and C(R⁴)(R⁴);
W⁵ is selected from N and CH;
L² is selected from —C(O)—;
R⁵ is a 5-6 membered heteroaryl, optionally substituted with one, two, or three R^{20k}, wherein the 5-6 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen, halogen, C₁₋₆alkyl, C₂₋₆alkenyl, C₂₋₆alkynyl, C₃₋₁₀cycloalkyl, C₂₋₉heterocycloalkyl, —OR¹², —SR¹², and —N(R¹²)(R¹³), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R^{8b} is selected from hydrogen, C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
L¹ is a bond;
R¹⁶ is independently selected from hydrogen, halogen, C₁₋₆alkyl, C₂₋₆alkenyl, C₂₋₆alkynyl, C₃₋₁₀cycloalkyl, C₂₋₉heterocycloalkyl, —OR¹², —SR¹², and —N(R¹²)(R¹³), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20g};
R² is selected from C₁₋₆alkyl, C₂₋₆alkenyl, C₂₋₆alkynyl, C₃₋₁₀cycloalkyl, C₂₋₉heterocycloalkyl, —OR^{12′}, —SR^{12′}, and —N(R^{12″})(R¹³), wherein said C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20d};
R¹⁸ is independently selected from hydrogen, halogen, C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20h}; and
[symbol] indicates a single or double bond such that all valences are satisfied.

In an aspect is provided a compound of Formula (Id), or a pharmaceutically acceptable salt or solvate thereof:

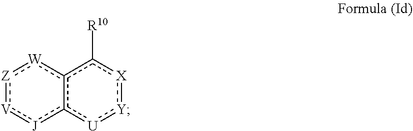

wherein
s1, s2, s3, R¹, R⁴, R⁶, R¹⁷, R^{12b}, R³, R¹², R^{12″}, R^{12c}, R¹³, R¹⁴, R^{14a}, R¹⁵, R^{20a}, R^{20b}, R^{20c}, R^{20d}, R^{20e}, R²⁰, R^{20g}, R^{20h}, R²⁰ⁱ, R^{20k}, R^{20m}, R²¹, R^{21b}, R²², R²³, R²⁴, and R²⁵ are as described for Formula (I′), including in embodiments of a compound of Formula (I′);
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

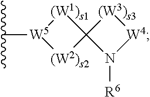

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
L² is —C(O)—; R⁵ is a 5 membered heteroaryl, optionally substituted with one, two, or three R^{20k}, wherein the 5 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen and halogen;
L¹ is a bond; R¹⁹ is selected from a C₆₋₁₂aryl and C₂₋₁₂heteroaryl, wherein the C₆₋₁₂aryl and C₂₋₁₂heteroaryl are optionally substituted with one, two, three, four, five, six, or seven R¹ⁱ; each R¹ⁱ is independently selected from halogen, oxo, —CN, C₁₋₆alkyl, C₁₋₆ haloalkyl, C₂₋₆alkenyl, C₂₋₆alkynyl, —OR¹², —N(R¹²)(R¹³), wherein C₁₋₆alkyl, C₂₋₆alkenyl, and C₂₋₆alkynyl are optionally substituted with one, two, or three R²⁰ⁱ;
R¹⁶ is independently selected from hydrogen and halogen; R² is —OR^{12′}; each R^{12′} is independently selected from —C(R^{12c})₂—C₂₋₉heterocycloalkyl, wherein —C(R^{12c})₂—C₂₋₉heterocycloalkyl is optionally substituted with one, two, or three R^{20d}; and
[symbol] indicates a single or double bond such that all valences are satisfied.

In an aspect is provided a compound of Formula (Id), or a pharmaceutically acceptable salt or solvate thereof:

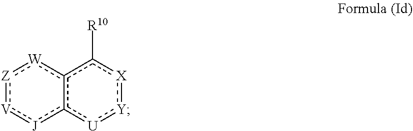

wherein
s1, s2, s3, R¹, R⁴, R⁶, R¹⁷, R^{12b}, R³, R¹², R^{12″}, R^{12c}, R¹³, R¹⁴, R^{14a}, R¹⁵, R^{20a}, R^{20b}, R^{20c}, R^{20d}, R^{20e}, R^{20f}, R^{20g}, R^{20h}, R²⁰ⁱ, R^{20m}, R²¹, R^{21b}, R²², R²³, R²⁴, and R²⁵ are as described for Formula (I′), including in embodiments of a compound of Formula (I′);
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

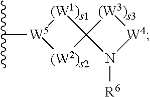

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
L² is —C(O)—; R⁵ is a 5 membered heteroaryl, optionally substituted with one, two, or three R^{20k}, wherein the 5 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen and halogen;
L¹ is a bond; R¹⁹ is selected from a C₆₋₁₂aryl and C₂₋₁₂heteroaryl, wherein the C₆₋₁₂aryl and C₂₋₁₂heteroaryl are optionally substituted with one, two, three, four, five, six, or seven R¹ⁱ; each R¹ⁱ is independently selected from halogen, oxo, —CN, C₁₋₆alkyl, C₁₋₆ haloalkyl, C₂₋₆alkenyl, C₂₋₆alkynyl, —OR¹², —N(R¹²)(R¹³), wherein C₁₋₆alkyl, C₂₋₆alkenyl, and C₂₋₆alkynyl are optionally substituted with one, two, or three R²⁰ⁱ;
R¹⁶ is independently selected from hydrogen and halogen; R² is —OR^{12′}; each R^{12′} is independently selected from —C(R^{12c})₂—C₂₋₉heterocycloalkyl, wherein —C(R^{12c})₂—C₂₋₉heterocycloalkyl is optionally substituted with one, two, or three R^{20d};
each R^{20k} is independently selected from halogen, —CN, —CH₂—CN, C₁₋₆alkyl, and C₃₋₁₀cycloalkyl, wherein C₁₋₆alkyl and C₃₋₁₀cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —SO₂N(R²²)(R²³), and —OC(O)R²⁵;
and [symbol] indicates a single or double bond such that all valences are satisfied.

In an aspect is provided a compound of Formula (Id), or a pharmaceutically acceptable salt or solvate thereof:

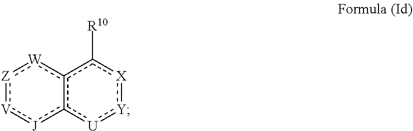

Wherein
s1, s2, s3, R¹, R⁴, R⁶, R¹⁷, R^{12b}, R³, R¹², R^{12″}, R^{12c}, R¹³, R¹⁴, R^{14a}, R¹⁵, R^{20a}, R^{20b}, R^{20c}, R^{20d}, R^{20e}, R²⁰, R^{20g}, R^{20h}, R²⁰ⁱ, R^{20m}, R²¹, R^{21b}, R²², R²³, R²⁴, and R²⁵ are as described for Formula (I′), including in embodiments of a compound of Formula (I′);
W is a CH; Z is N or C(R^{g}); V is C(R¹⁷); J is C(R¹⁶); Y is C(R²); U is N; X is N;
R¹⁰ is -L⁷-R⁷;
L⁷ is a bond;

R⁷ is

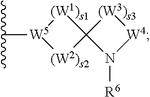

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
L² is —C(O)—; R⁵ is a 5 membered heteroaryl, optionally substituted with one, two, or three R^{20k}, wherein the 5 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen and halogen;
L¹ is a bond; R¹⁹ is selected from a C₆₋₁₂aryl and C₂₋₁₂heteroaryl, wherein the C₆₋₁₂aryl and C₂₋₁₂heteroaryl are optionally substituted with one, two, three, four, five, six, or seven R¹ⁱ; each R¹ⁱ is independently selected from halogen, oxo, —CN, C₁₋₆alkyl, C₁₋₆ haloalkyl, C₂₋₆alkenyl, C₂₋₆alkynyl, —OR¹², —N(R¹²)(R¹³), wherein C₁₋₆alkyl, C₂₋₆alkenyl, and C₂₋₆alkynyl are optionally substituted with one, two, or three R²⁰ⁱ;
R¹⁶ is independently selected from hydrogen and halogen; R² is —OR^{12′}; each R^{12′} is independently selected from —C(R^{12c})₂—C₂₋₉heterocycloalkyl, wherein —C(R^{12c})₂—C₂₋₉heterocycloalkyl is optionally substituted with one, two, or three R^{20d};
each R^{20k} is independently selected from halogen, —CN, —CH₂—CN, C₁₋₃alkyl, and C₃₋₄cycloalkyl, wherein C₁₋₃alkyl and C₃₋₄cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
and [symbol] indicates a single or double bond such that all valences are satisfied.

In an aspect is provided a compound of Formula (Ie), or a pharmaceutically acceptable salt or solvate thereof:

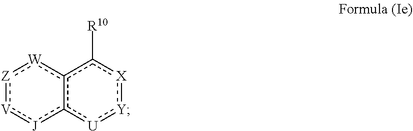

Wherein
s1, s2, s3, R¹, R⁴, R⁶, R¹², R¹³, R¹⁴, R^{14a}, R¹⁵, R^{20a}, R²¹, R^{21b}, R²², R²³, R²⁴, and R²⁵ are as described for Formula (I′), including in embodiments of a compound of Formula (I′);
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

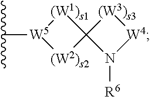

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH; L² is —C(O)—;
R⁵ is selected from

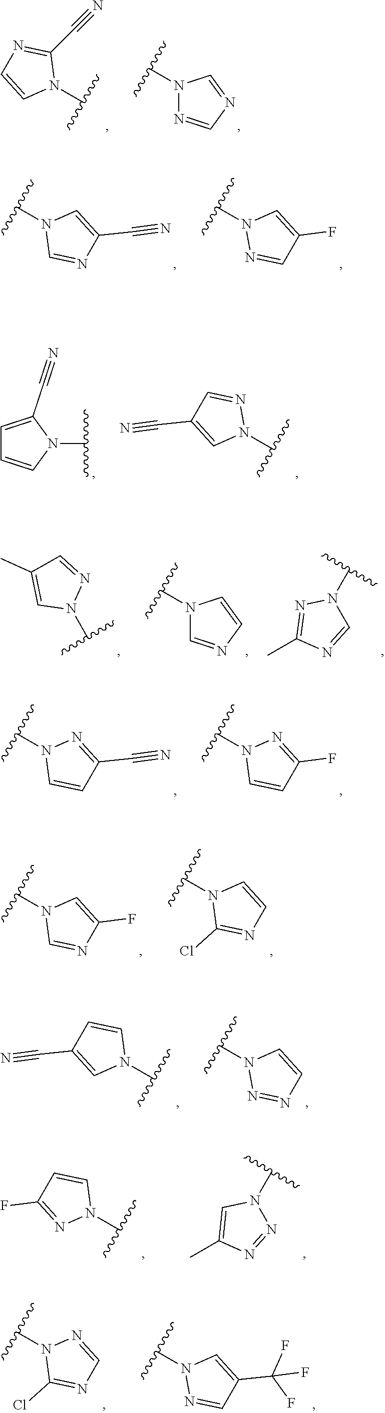

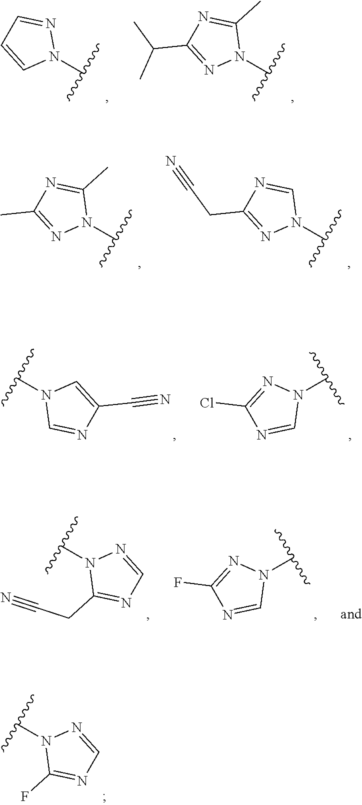

R⁸ is independently selected from

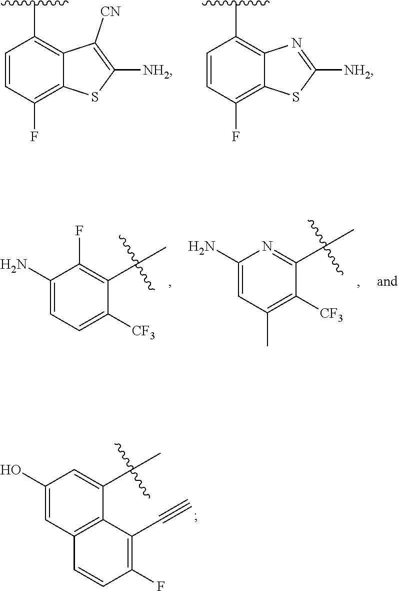 [SMILES: *c1ccc(F)c2sc(N)c(C#N)c12 ; *c1ccc(F)c2sc(N)nc12 ; *c1c(C(F)(F)F)ccc(N)c1F ; *c1nc(N)cc(C)c1C(F)(F)F ; *c1cc(O)cc2ccc(F)c(C#C)c12]

R¹⁶ is independently selected from hydrogen and halogen; R² is selected from

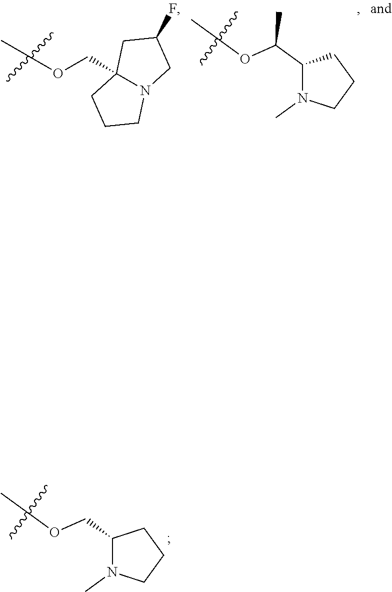

and [symbol] indicates a single or double bond such that all valences are satisfied.

In an aspect is provided a compound of Formula (If), or a pharmaceutically acceptable salt or solvate thereof:

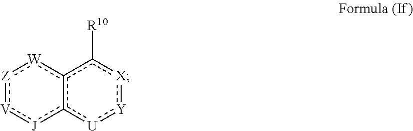

Wherein
s1, s2, s3, R¹, R⁴, R⁶, R¹², R¹³, R¹⁴, R^{14a}, R¹⁵, R^{20a}, R²¹, R^{21b}, R²², R²³, R²⁴, and R²⁵ are as described for Formula (I′), including in embodiments of a compound of Formula (I′);
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

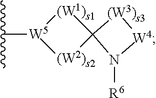

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH; L² is —C(O)—;
R⁵ is selected from

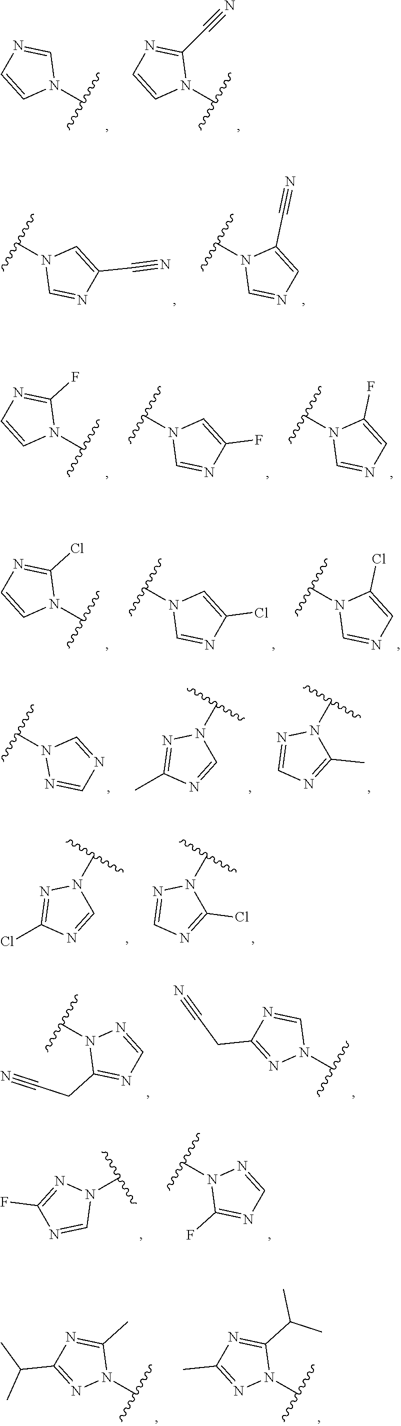

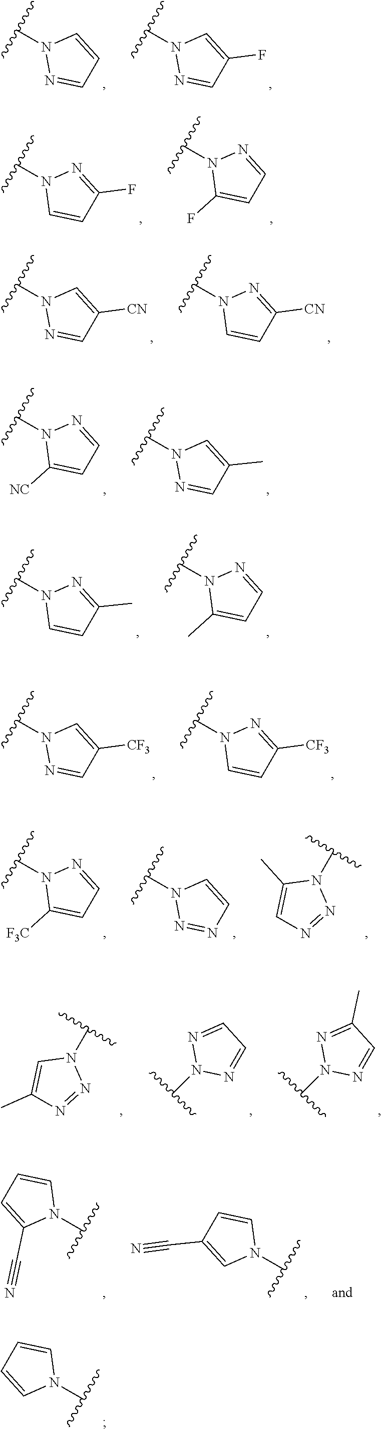

R⁸ is independently selected from hydrogen and halogen; R¹⁷ is selected from

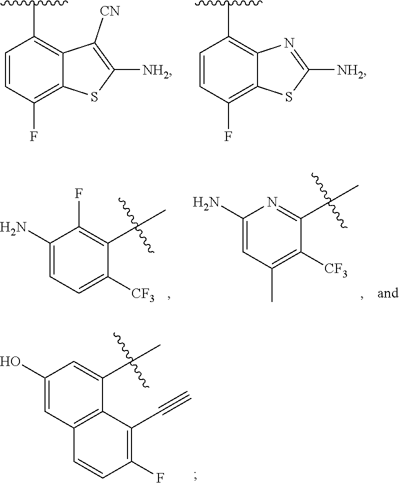 [SMILES: *c1ccc(F)c2sc(N)c(C#N)c12 ; *c1ccc(F)c2sc(N)nc12 ; *c1c(C(F)(F)F)ccc(N)c1F ; *c1nc(N)cc(C)c1C(F)(F)F ; *c1cc(O)cc2ccc(F)c(C#C)c12]

R¹⁶ is independently selected from hydrogen and halogen; R² is selected from

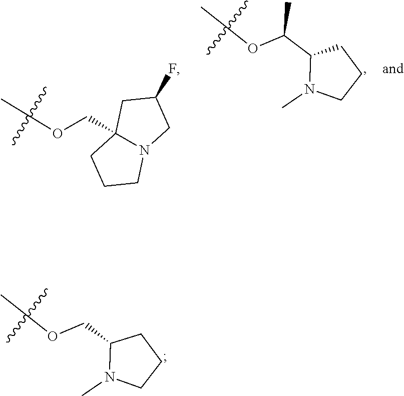

[symbol]

indicates a single or double bond such that all valences are satisfied.

In an aspect is provided a compound of Formula (Ig), or a pharmaceutically acceptable salt or solvate thereof:

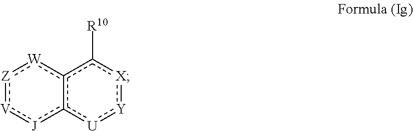

Wherein
R¹, R⁴, R⁶, R¹², R¹³, R¹⁴, R^{14a}, R¹⁵, R^{20a}, R²OC, R²¹, R^{21b}, R²², R²³, R²⁴, and R²⁵ are as described for Formula (I′), including in embodiments of a compound of Formula (I′);
W is a CH or C(O); Z is N, C(R¹), or N(R^{1b}); V is C(R¹⁷); J is C(R¹⁶); Y is C(R²); U is N; X is N;
R¹⁰ is -L⁷-R⁷;
L⁷ is a bond;
R⁷ is selected from

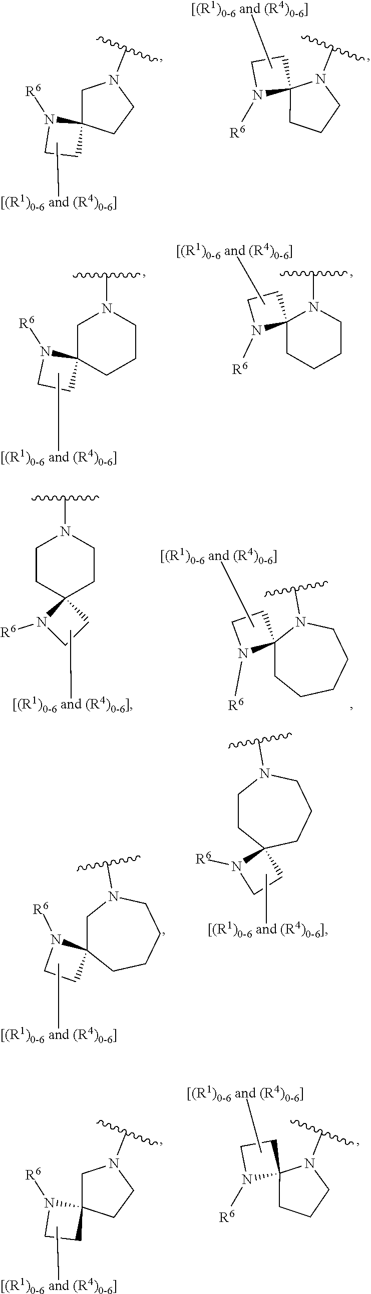

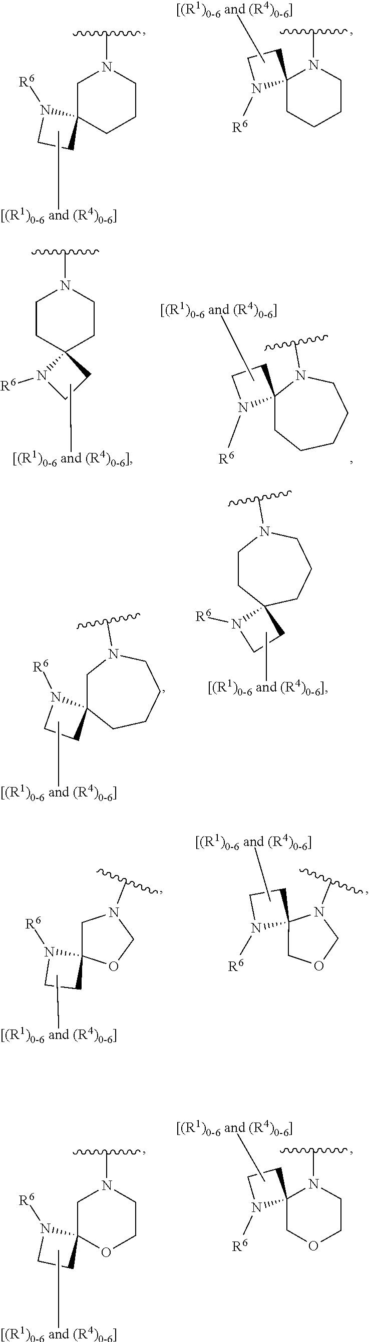

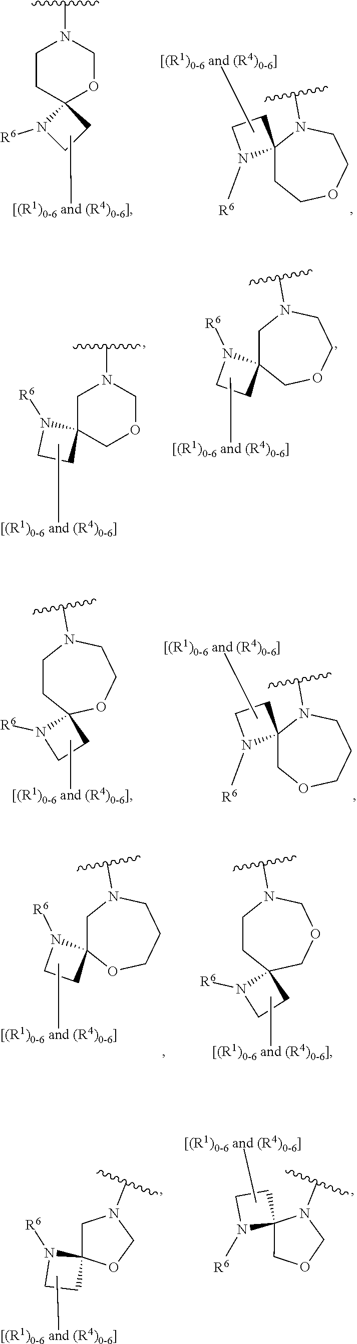

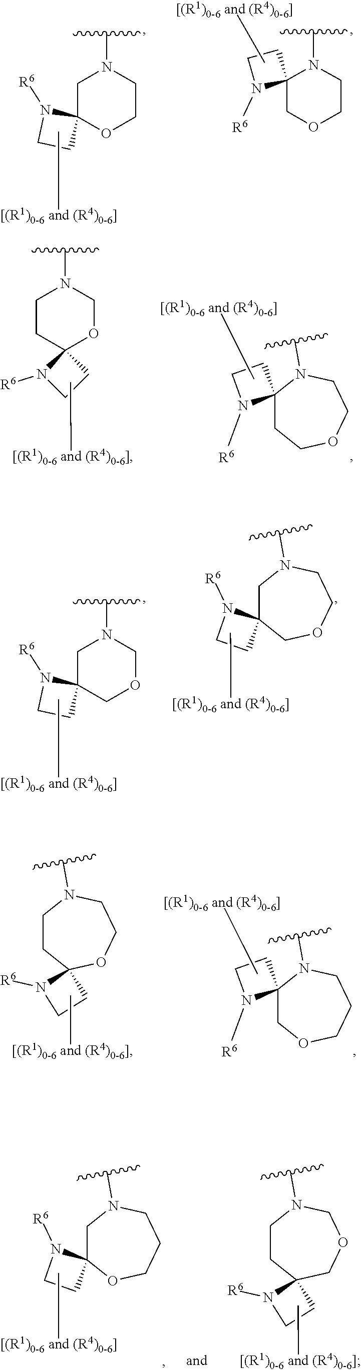

L² is —C(O)—, R⁵ is a selected from

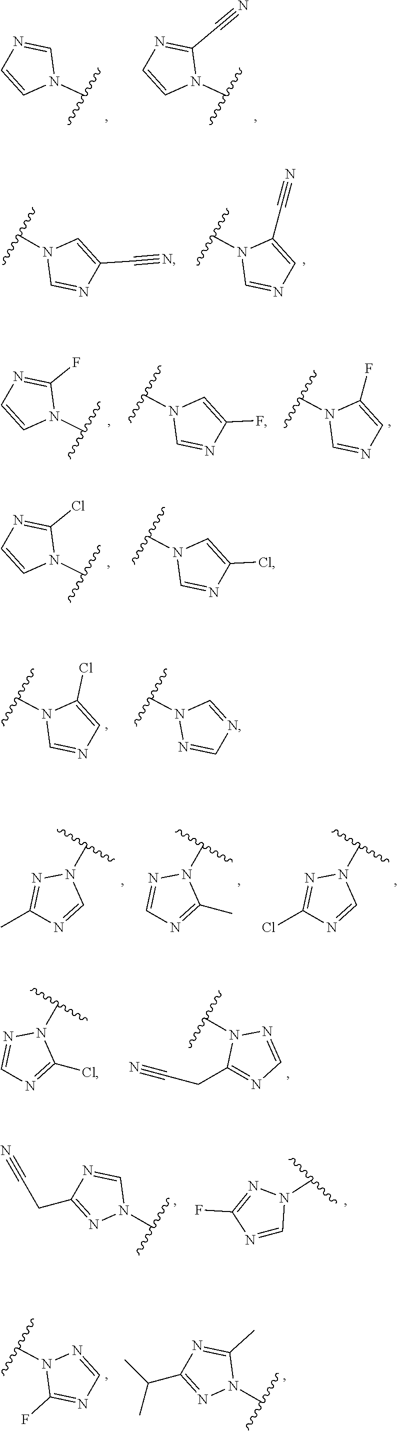

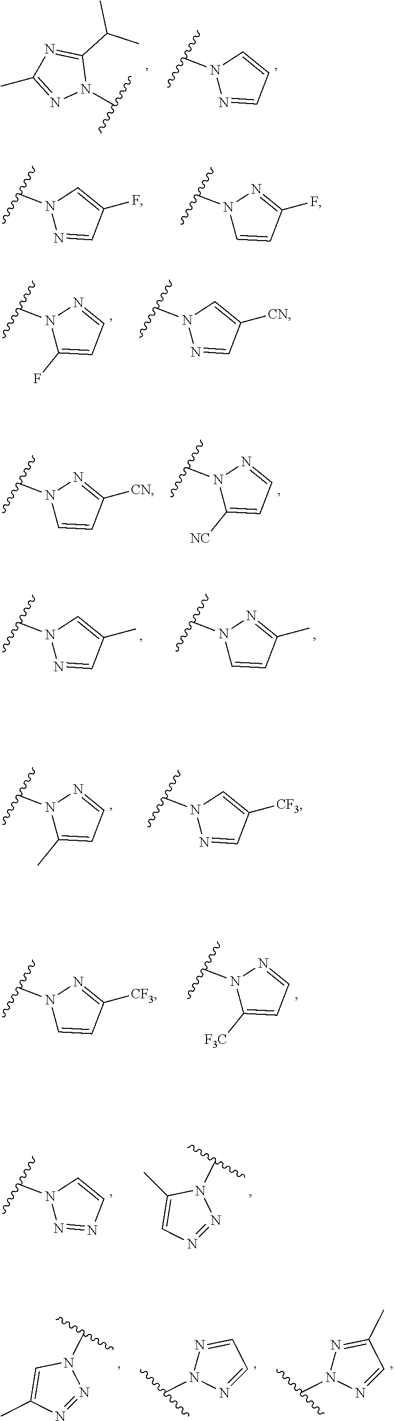

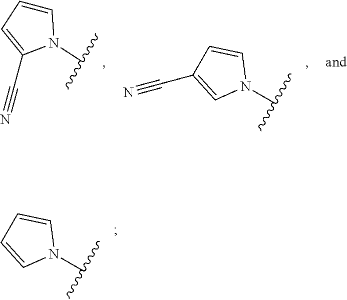 [SMILES: *n1cccc1C#N ; *n1ccc(C#N)c1 ; *n1cccc1]

R⁸ is independently selected from hydrogen or halogen;
R^{8b} is selected from C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R¹⁷ is selected from

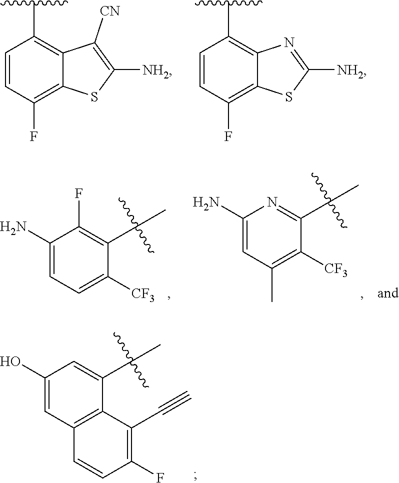 [SMILES: *c1ccc(F)c2sc(N)c(C#N)c12 ; *c1ccc(F)c2sc(N)nc12 ; *c1c(C(F)(F)F)ccc(N)c1F ; *c1nc(N)cc(C)c1C(F)(F)F ; *c1cc(O)cc2ccc(F)c(C#C)c12]

R¹⁶ is independently selected from hydrogen and halogen; R² is selected from

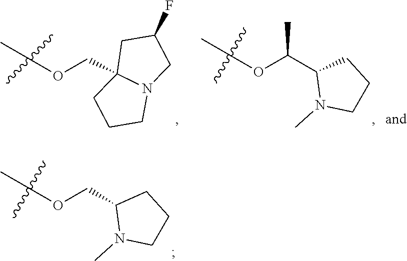

and [symbol] indicates a single or double bond such that all valences are satisfied.

In embodiments of Formula (Ig), [(R¹)₀₋₆ and (R⁴)₀₋₆] is equal to R⁴, and R⁴ is selected from halogen, methyl, and —CH₂CN. In embodiments of Formula (Ig), [(R¹)₀₋₆ and (R⁴)₀₋₆] is equal to [(R¹)₀ and (R⁴)O] (i.e., no R¹ or R⁴ substituent). In embodiments of Formula (Ig), [(R¹)₀₋₆ and (R⁴)₀₋₆] is equal to two R⁴, and each R⁴ is independently selected from halogen, methyl, and —CH₂CN.

In embodiments of Formula (I), (I-3), (I-4), (II-3), (I′), (Ia), (Ib), (Ic), (Id), (Ie), (If), (Ig), (II), (II′), (II″), (IV-3), (IV-4), (IV), (IV′), or (IVa), the compound has a formula selected from:

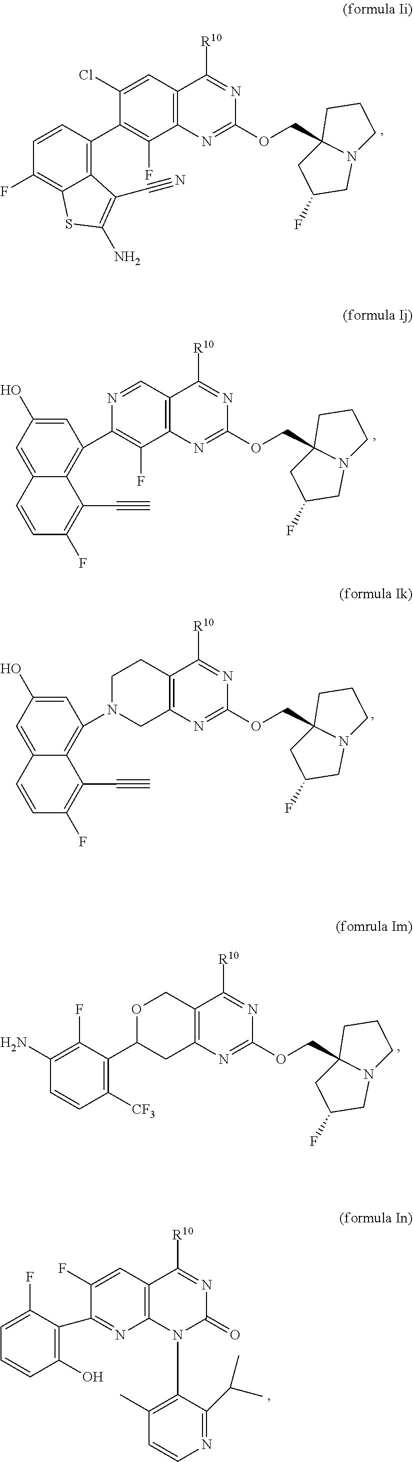

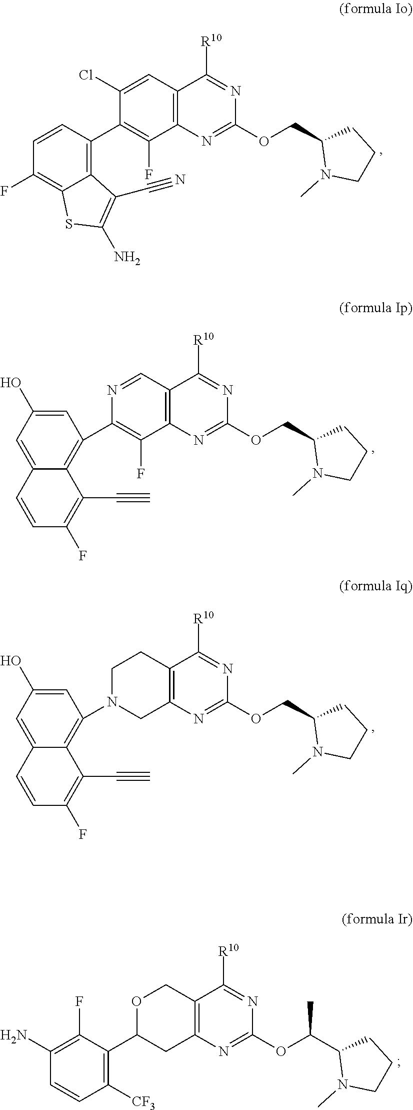

wherein R¹⁰ is as described herein, including in any aspect or embodiment.

In embodiments of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″),
R⁷ is

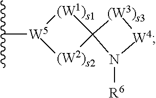

at least one W³ and/or W⁴ is C(R¹)(R¹), C(R¹)(R⁴), or C(R⁴)(R⁴);
R¹ and R⁴ are as described for Formula (I′), including in embodiments of a compound of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″); and at least one i) R¹ of W³ or W⁴ is not hydrogen or ii) one R⁴ of W³ or W⁴ is not hydrogen.

In embodiments of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″),
R⁷ is

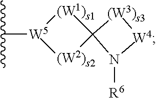

at least one W³ and/or W⁴ is C(R¹)(R¹), C(R¹)(R⁴), or C(R⁴)(R⁴).
R¹ and R⁴ are as described for Formula (I′), including in embodiments of a compound of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″). In embodiments, R¹ and R⁴ are hydrogen.

In embodiments of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″),
R⁷ is

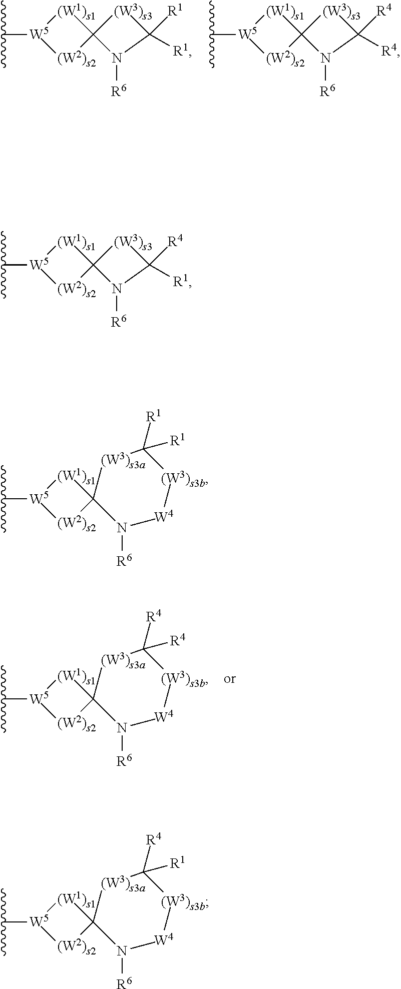

R¹ and R⁴ are as described for Formula (I′), including in embodiments of a compound of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″);
at least one R¹ or R⁴ shown in the formulae immediately above is not hydrogen; and
s3a is 0, 1, or 2; s3b is 0, 1, or 2; the sum of s3a and s3b is 0, 1, or 2.

In embodiments of Formula (IV-3), (IV-4), (IV), (IVa), or (IV′),
R⁷ is

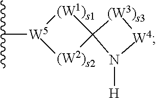

at least one W³ and/or W⁴ is C(R¹)(R¹), C(R¹)(R⁴), or C(R⁴)(R⁴).
R¹ and R⁴ are as described for Formula (I′), including in embodiments of a compound of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″); and at least one i) R¹ of W³ or W⁴ is not hydrogen or ii) one R⁴ of W³ or W⁴ is not hydrogen.

In embodiments of Formula (IV-3), (IV-4), (IV), (IVa), or (IV′),
R⁷ is

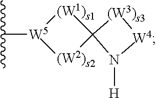

at least one W³ and/or W⁴ is C(R¹)(R¹), C(R¹)(R⁴), or C(R⁴)(R⁴).
R¹ and R⁴ are as described for Formula (I′), including in embodiments of a compound of Formula (I), (I-3), (I-4), (II-3), (I′), (Ia), (Ib), (Ic), (Id), (Ie), (If), (Ii), (Ij), (Ik), (Im), (In), (Io), (Ip), (Iq), (Ir), (II), (II′), or (II″). In embodiments, R¹ and R⁴ are hydrogen.

In embodiments of Formula (IV-3), (IV-4), (IV), (IVa), or (IV′),
R⁷ is

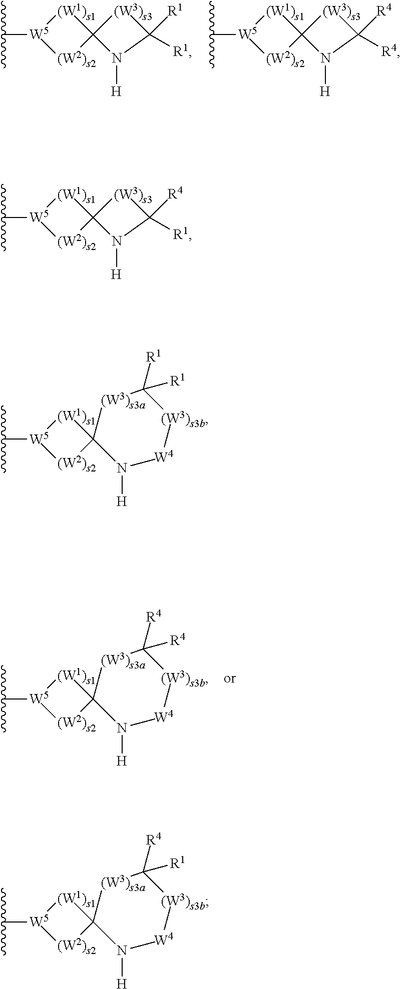

R¹ and R⁴ are as described for Formula (I′), including in embodiments of a compound of Formula (IV-3), (IV-4), (IV), (IVa), or (IV′);
at least one R¹ or R⁴ shown in the formulae immediately above is not hydrogen; and
s3a is 0, 1, or 2; s3b is 0, 1, or 2; the sum of s3a and s3b is 0, 1, or 2.

In an aspect is provided a compound of Formula (I-3), or a pharmaceutically acceptable salt or solvate thereof:

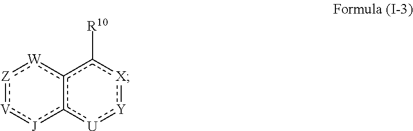

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), S(O), and S(O)₂;
s1 is 1 and s2 is 0, 2, or 3; s1 is 2 and s2 is 0, 1, or 3; s1 is 3 and s2 is 0 to 3; s1 is 4 and s2 is 0 to 3; s1 is 5 and s2 is 0 to 3; or s1 is 6 and s2 is 0 to 3; and
all other variables of the formula (e.g., W, Z, V, J, Y, U, R¹⁰, L⁷, R⁷, W¹, W³, W⁵, s3, R¹, R⁶, L², R⁴, R⁵, R², R^{4c}, R^{4d}, R⁸, R^{8a}, R^{Bb}, R¹⁷, R^{17b}, L¹, L^{1b}, R^{1e}, R^{1f}, R^{1g}, R^{1f}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R¹⁴, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (I), or embodiments thereof.

In an aspect is provided a compound of Formula (I-3), or a pharmaceutically acceptable salt or solvate thereof:

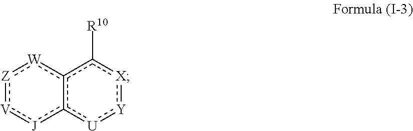

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), S(O), and S(O)₂;
s1 is an integer from 1 to 6;
s2 is an integer from 0 to 3;
s1 and s2 are not both 1; s1 and s2 are not both 2; and
all other variables of the formula (e.g., W, Z, V, J, Y, U, R¹⁰, L⁷, R⁷, W¹, W³, W⁵, s3, R¹, R⁶, L², R⁴, R⁵, R², R^{4c}, R^{4d}, R⁸, R^{8a}, R^{8b}, R¹⁷, R^{17b}, L¹, L^{1b}, R^{1e}, R^{1f}, R^{1g}, R^{1f}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (I), or embodiments thereof.

In an aspect is provided a compound of Formula (I-4), or a pharmaceutically acceptable salt or solvate thereof:

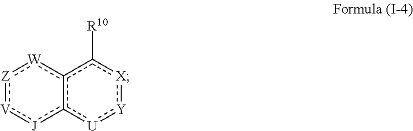

Wherein
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), ═C(R^{21b})₂, —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl), —S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl)-S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁ heteroaryl are optionally substituted with one, two, or three R^{20k};
R² is selected from halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉ heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₄₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₄₋₉heterocycloalkyl, C₆₋₁₀aryl, and C₁₋₉heteroaryl are optionally substituted with one, two, or three R^{20d};
each R^{20a}, R^{20b}, R^{20c}, R^{20d}, R^{20e}, R^{20f}, R^{20g}, R^{20h}, R²⁰ⁱ, R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), ═C(R^{21b})₂, —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²¹, —N(R²⁴)S(O)₂R²⁵, —C(O)R²¹, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
[symbol] indicates the location of attachment of the depicted chemical formula or atom to a substituent, a further component of a molecule, or an atom;
R^{21b} is independently selected at each occurrence from hydrogen, halogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, or two R^{21b} are taken together with the carbon atom to which they are attached to form C₃₋₁₀cycloalkyl or C₂₋₉heterocycloalkyl; each of which is optionally substituted with one, two, or three substituents independently selected from halogen, C₁₋₃ alkyl, C₁₋₃ haloalkyl, and —OH;
[symbol] indicates a single or double bond such that all valences are satisfied; and
all other variables of the formula (e.g., W, Z, V, J, Y, U, R¹⁰, L⁷, R⁷, W¹, W³, W², W⁴, W⁵, s1, s2, s3, R¹, R⁶, L², R^{4c}, R^{4d}, R⁸, R^{8a}, R^{8b}, R¹⁷, R^{17b}, L¹, L^{1b}, R^{1e}, R^{1f}, R^{1g}, R^{1c}, R¹⁹, R¹ⁱ, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R²¹, R²², R²³, R², and R²⁵) are as described for Formula (I-3), or embodiments thereof.

In another aspect is provided a compound of Formula (Ia), or a pharmaceutically acceptable salt or solvate thereof:

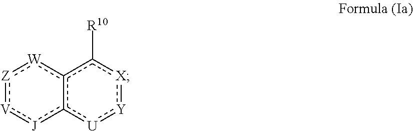

Wherein
Y is N, C(R²), C(R²)(R^{2c}), N(R^{2b}), S(O), S(O)₂, or C(O);
W² is independently selected from N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), and C(R⁴)(R⁴);
s1 is 1 and s2 is 0, 2, or 3; s1 is 2 and s2 is 0 to 3; s1 is 3 and s2 is 0 to 3; s1 is 4 and s2 is 0 to 3; s1 is 5 and s2 is 0 to 3; or s1 is 6 and s2 is 0 to 3;
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R¹⁹ is selected from C₂₋₁₁heterocycloalkyl and C₂₋₁₂heteroaryl, wherein the C₂₋₁₁heterocycloalkyl and C₂₋₁₂heteroaryl are optionally substituted with one, two, three, four, five, six, or seven R¹ⁱ;
[symbol] indicates a single or double bond such that all valences are satisfied; and
all other variables of the formula (e.g., W, Z, V, J, U, R¹¹, L⁷, R⁷, W¹, W³, W², W⁵, s3, R¹, R⁴, R⁶, L², R⁵, R^{4c}, R^{4a}, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In another aspect is provided a compound of Formula (Ib), or a pharmaceutically acceptable salt or solvate thereof:

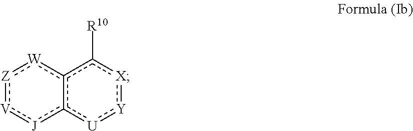

wherein
W² is independently selected from N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), S(O), and S(O)₂;
s1 is 1 and s2 is 0, 2, or 3; s1 is 2 and s2 is 0 to 3; s1 is 3 and s2 is 0 to 3; s1 is 4 and s2 is 0 to 3; s1 is 5 and s2 is 0 to 3; or s1 is 6 and s2 is 0 to 3; and
Y is N, C(R²), C(R²)(R^{2c}), N(R^{2b}), S(O), S(O)₂, or C(O);
R² is hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C_{2- 9}heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R¹⁹ is:

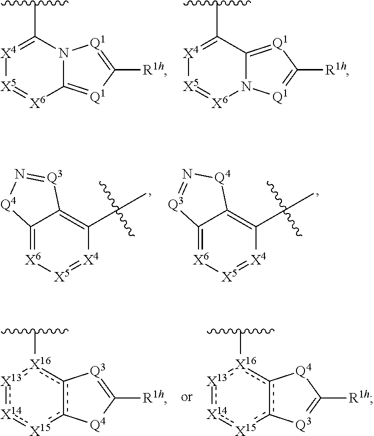

Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, X⁸ are independently selected from a C(O), C(R^{1a}), N, C(R^{1a})(R^{1b}), and N(R^{1c});
X¹⁶ are independently selected from C, N, and C(R^{1a});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ; or two Ria bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R¹¹, and R^{id} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ;
each R^{1e} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
[symbol] indicates a single or double bond such that all valences are satisfied; and
all other variables of the formula (e.g., W, Z, V, J, U, R¹⁰, L⁷, R⁷, W¹, W³, W², W⁵, s3, R¹, R⁴, R⁶, L², R⁸, R^{4c}, R^{4d}, R⁸, R^{8a}, R^{8b}, R¹⁷, R¹⁷, L¹, L^{1b}, R^{1e}, R^{1f}, R^{1g}, R^{1c}, R¹ⁱ, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In an aspect is provided a compound of Formula (Ic), or a pharmaceutically acceptable salt or solvate thereof:

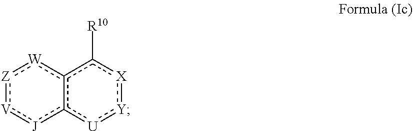

wherein
W is a C(R¹⁸) or C(O); Z is N, C(R⁸), or N(R^{8b}); V is C(R¹⁷); J is C(R¹⁶); Y is C(R²); U is N; X is N;
R¹⁰ is -L⁷-R⁷;
L⁷ is a bond;
R⁷ is

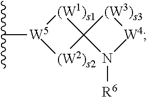

W¹, W², and W³ are independently selected from C(R¹)(R¹), C(R¹)(R⁴), C(R⁴)(R⁴), and O;
W⁴ is selected from C(R¹)(R¹), C(R¹)(R⁴), and C(R⁴)(R⁴);
W⁵ is selected from N and CH;
s1 is an integer from 1 to 6;
s2 is an integer from 1 to 3;
s3 is an integer from 1 to 3;
L² is selected from —C(O)—;
R⁵ is a 5-6 membered heteroaryl, optionally substituted with one, two, or three R^{20k}, wherein the 5-6 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen, halogen, C₁₋₆alkyl, C₂₋₆alkenyl, C₂₋₆alkynyl, C₃₋₁₀cycloalkyl, C₂₋₉heterocycloalkyl, —OR¹², —SR¹², and —N(R¹²)(R¹³), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R^{8b} is selected from hydrogen, C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
L¹ is a bond;
R¹⁶ is independently selected from hydrogen, halogen, C₁₋₆alkyl, C₂₋₆alkenyl, C₂₋₆alkynyl, C₃₋₁₀cycloalkyl, C₂₋₉heterocycloalkyl, —OR¹², —SR¹², and —N(R¹²)(R¹³), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20g};
R² is selected from C₁₋₆alkyl, C₂₋₆alkenyl, C₂₋₆alkynyl, C₃₋₁₀cycloalkyl, C₂₋₉heterocycloalkyl, —OR^{12′}, —SR^{12′}, and —N(R^{12″})(R¹³), wherein said C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20d};
R¹⁸ is independently selected from hydrogen, halogen, C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20h};
[symbol] indicates a single or double bond such that all valences are satisfied; and
all other variables of the formula (e.g., R¹, R⁴, R⁶, R^{4c}, R^{4d}, R¹⁷, L^{1b}, R^{1e}, R^{1f}, R^{1g}, R^{1c}, R¹⁹, R¹ⁱ, R^{16a}, R^{16b}, R^{2c}, R^{2b}, R^{12b}, R³, R¹², R^{12′}, R^{12″}, R^{12c}, R¹³, R¹⁴, R^{14a}, R¹⁵, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In an aspect is provided a compound of Formula (Id), or a pharmaceutically acceptable salt or solvate thereof:

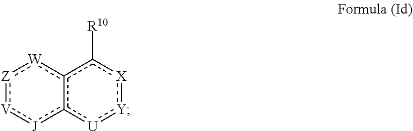

wherein
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

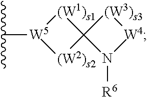

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
s1 is an integer from 1 to 6;
s2 is an integer from 1 to 3;
s3 is an integer from 1 to 3;
L² is —C(O)—; R⁵ is a 5 membered heteroaryl, optionally substituted with one, two, or three R^{20k}, wherein the 5 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen and halogen;
L¹ is a bond; R¹⁹ is selected from a C₆₋₁₂aryl and C₂₋₁₂heteroaryl, wherein the C₆₋₁₂aryl and C₂₋₁₂heteroaryl are optionally substituted with one, two, three, four, five, six, or seven R¹ⁱ; each R¹ⁱ is independently selected from halogen, oxo, —CN, C₁₋₆alkyl, C₁₋₆haloalkyl, C₂₋₆alkenyl, C₂₋₆alkynyl, —OR¹², —N(R¹²)(R¹³), wherein C₁₋₆alkyl, C₂₋₆alkenyl, and C₂₋₆alkynyl are optionally substituted with one, two, or three R²⁰ⁱ;
R¹⁶ is independently selected from hydrogen and halogen; R² is —OR^{12′}; each R^{12′} is independently selected from —C(R^{12c})₂—C_{2 -9}heterocycloalkyl, wherein —C(R^{12c})₂—C₂₋₉heterocycloalkyl is optionally substituted with one, two, or three R^{20d};
[symbol] indicates a single or double bond such that all valences are satisfied; and
all other variables of the formula (e.g., R¹, R⁴, R⁶, R¹⁷, R^{12b}, R³, R¹², R^{12″}, R^{12c}, R¹³, R¹⁴, R^{14a}, R¹⁵, R^{20a}, R^{20b}, R^{20c}, R^{20d}, R^{20e}, R²⁰, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In an aspect is provided a compound of Formula (Id), or a pharmaceutically acceptable salt or solvate thereof:

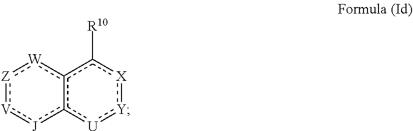

wherein
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

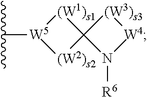

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
s1 is an integer from 1 to 6;
s2 is an integer from 1 to 3;
s3 is an integer from 1 to 3;
L² is —C(O)—; R⁵ is a 5 membered heteroaryl, optionally substituted with one, two, or three R^{20k}, wherein the 5 membered heteroaryl comprises one, two, or three ring nitrogen atoms and further wherein when R⁵ is directly bonded to a C(O) of L², L² is directly bonded to an N atom of R⁵;
R⁸ is independently selected from hydrogen and halogen;
L¹ is a bond; R¹⁹ is selected from a C₆₋₁₂aryl and C₂₋₁₂heteroaryl, wherein the C₆₋₁₂aryl and C₂₋₁₂heteroaryl are optionally substituted with one, two, three, four, five, six, or seven R¹ⁱ; each R¹ⁱ is independently selected from halogen, oxo, —CN, C₁₋₆alkyl, C₁₋₆haloalkyl, C₂₋₆alkenyl, C₂₋₆alkynyl, —OR¹², —N(R¹²)(R¹³), wherein C₁₋₆alkyl, C₂₋₆alkenyl, and C₂₋₆alkynyl are optionally substituted with one, two, or three R²⁰ⁱ;
R¹⁶ is independently selected from hydrogen and halogen; R² is —OR^{12′}; each R^{12′} is independently selected from —C(R^{12c})₂—C₂₋₉heterocycloalkyl, wherein —C(R^{12c})₂—C₂₋₉heterocycloalkyl is optionally substituted with one, two, or three R^{20d};
each R^{20k} is independently selected from halogen, —CN, —CH₂—CN, C₁₋₆alkyl, and C₃₋₁₀cycloalkyl, wherein C₁₋₆alkyl and C₃₋₁₀cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
[symbol] indicates a single or double bond such that all valences are satisfied; and
all other variables of the formula (e.g., R¹, R⁴, R⁶, R¹⁷, R^{12b}, R³, R¹², R^{12″}, R^{12c}, R¹³, R¹⁴, R^{14a}, R¹⁵, R^{20a}, R^{20b}, R^{20c}, R^{20d}, R^{20e}, R^{20f}, R^{20g}, R^{20h}, R²⁰ⁱ, R^{20m}, R²¹, R^{21b}, R²², R²³, R²⁴, and R²⁵) are as described for Formula (I-4), or embodiments thereof.

In an aspect is provided a compound of Formula (Id), or a pharmaceutically acceptable salt or solvate thereof:

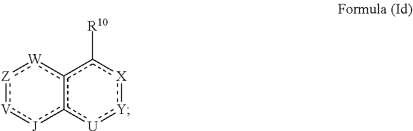

Wherein
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

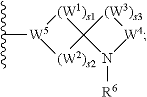

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
s1 is an integer from 1 to 6;
s2 is an integer from 1 to 3;
s3 is an integer from 1 to 3;
L² is —C(O)—; R⁵ is a 5 membered heteroaryl, optionally substituted with one, two, or three R^{20k}, wherein the 5 membered heteroaryl comprises one, two, or three ring nitrogen atoms and further wherein when R⁵ is directly bonded to a C(O) of L², L² is directly bonded to an N atom of R⁵;
R¹ is independently selected from hydrogen and halogen;
L¹ is a bond; R¹⁹ is selected from a C₆₋₁₂aryl and C₂₋₁₂heteroaryl, wherein the C₆₋₁₂aryl and C₂₋₁₂heteroaryl are optionally substituted with one, two, three, four, five, six, or seven R¹ⁱ; each R¹ⁱ is independently selected from halogen, oxo, —CN, C₁₋₆alkyl, C₁₋₆ haloalkyl, C₂₋₆alkenyl, C₂₋₆alkynyl, —OR¹², —N(R¹²)(R¹³), wherein C₁₋₆alkyl, C₂₋₆alkenyl, and C₂₋₆alkynyl are optionally substituted with one, two, or three R²⁰ⁱ;
R¹⁶ is independently selected from hydrogen and halogen; R² is —OR^{12′}; each R^{12′} is independently selected from —C(R^{12c})₂—C₂₋₉heterocycloalkyl, wherein —C(R^{12c})₂—C₂₋₉heterocycloalkyl is optionally substituted with one, two, or three R²⁰;
each R^{20k} is independently selected from halogen, —CN, —CH₂—CN, C₁₋₃alkyl, and C₃₋₄cycloalkyl, wherein C₁₋₃alkyl and C₃₋₄cycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R⁴)S(O)₂R²⁵, —C(O)R²⁵, —S(O)₂R²⁵, —S(O)₂N(R²²)(R²³), and —OC(O)R²⁵;
[symbol] indicates a single or double bond such that all valences are satisfied;
all other variables of the formula (e.g., R¹, R⁴, R⁶, R¹⁷, R^{12b}, R³, R¹², R^{12″}, R^{12c}, R¹³, R¹⁴, R^{14a}, R¹⁵, R^{20a}, R^{20b}, R^{20e}, R^{20d}, R^{20c}, R^{20f}, R^{20g}, R^{20h}, R²⁰ⁱ, R^{20m}, R²¹, R^{21b}, R²², R²³, R²⁴, and R²⁵) are as described for Formula (I-4), or embodiments thereof.

In an aspect is provided a compound of Formula (Ie), or a pharmaceutically acceptable salt or solvate thereof:

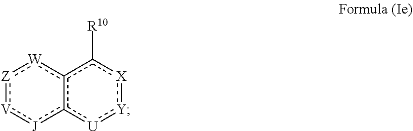

wherein
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

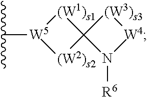

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
s1 is an integer from 1 to 6;
s2 is an integer from 1 to 3;
s3 is an integer from 1 to 3;
L² is —C(O)—;
R⁵ is selected from

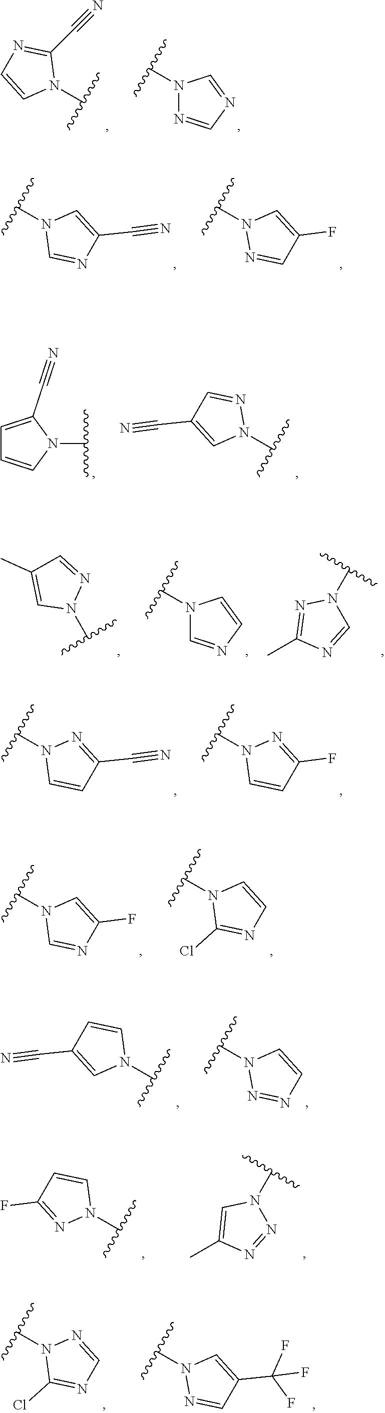

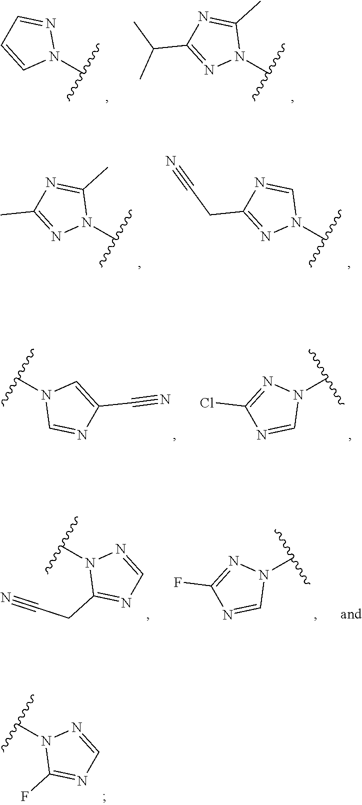

R⁸ is independently selected from hydrogen and halogen; R¹⁷ is selected from

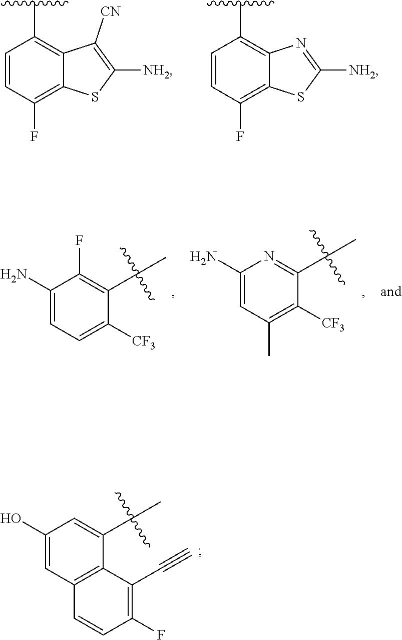 [SMILES: *c1ccc(F)c2sc(N)c(C#N)c12 ; *c1ccc(F)c2sc(N)nc12 ; *c1c(C(F)(F)F)ccc(N)c1F ; *c1nc(N)cc(C)c1C(F)(F)F ; *c1cc(O)cc2ccc(F)c(C#C)c12]

R¹⁶ is independently selected from hydrogen and halogen; R² is selected from

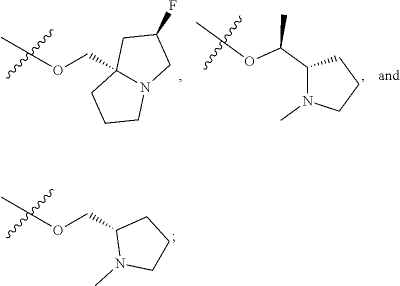

[symbol] indicates a single or double bond such that all valences are satisfied;
all other variables of the formula (e.g., R¹, R⁴, R⁶, R¹², R¹³, R¹⁴, R^{14a}, R¹⁵, R^{20a}, R²¹, R^{21b}, R²², R²³, R²⁴, and R²⁵) are as described for Formula (I-4), or embodiments thereof.

In an aspect is provided a compound of Formula (If), or a pharmaceutically acceptable salt or solvate thereof:

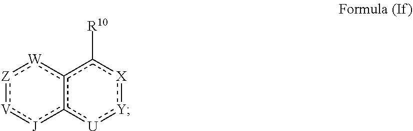

wherein
W is a CH; Z is N or C(R⁸); V is C(R¹⁷); J is C(R¹⁶); Y is C(R²); U is N; X is N;
R¹⁰ is -L⁷-R⁷;
L⁷ is a bond;
R⁷ is

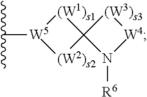

W¹, W², and W³ are independently selected from C(R¹)(R¹), C(R¹)(R⁴), C(R⁴)(R⁴), and O; W⁴ is selected from C(R¹)(R¹), C(R¹)(R⁴), and C(R⁴)(R⁴); W⁵ is selected from N and CH;
s1 is an integer from 1 to 6;
s2 is an integer from 1 to 3;
s3 is an integer from 1 to 3;
L² is —C(O)—;
R⁵ is selected form

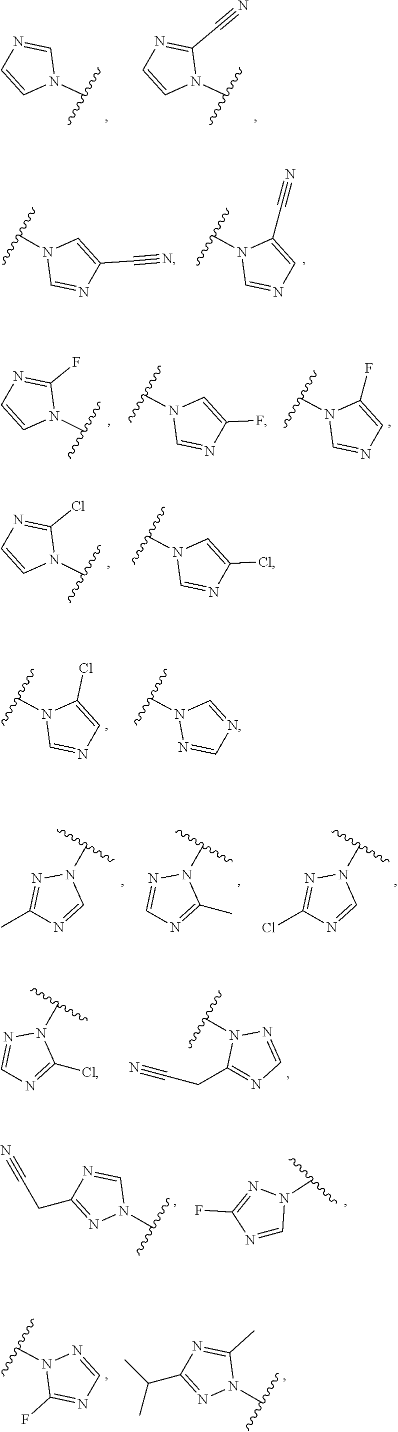

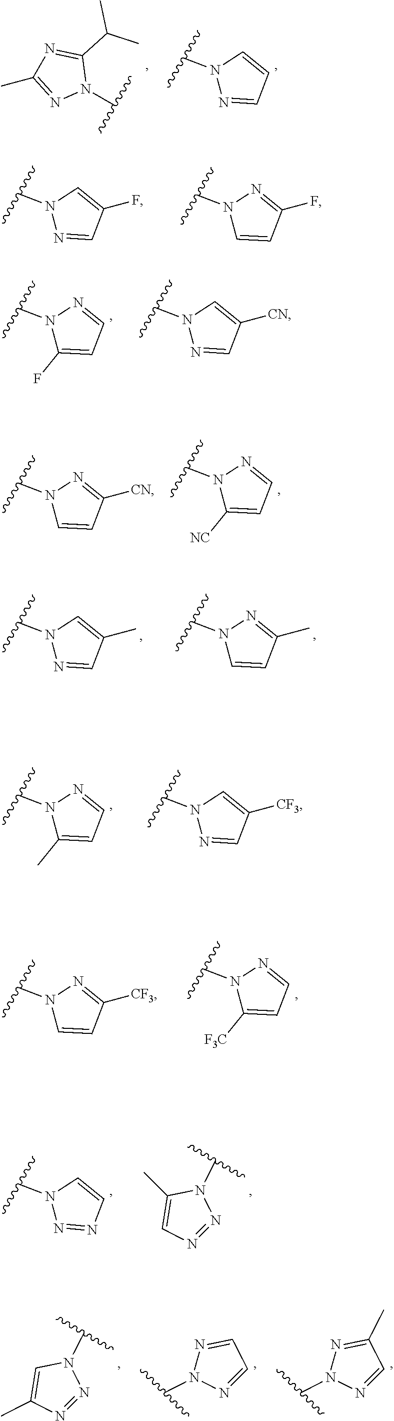

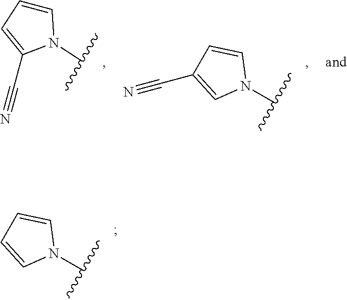 [SMILES: *n1cccc1C#N ; *n1ccc(C#N)c1 ; *n1cccc1]

R¹⁸ is independently selected from hydrogen and halogen; R¹⁷ is selected from

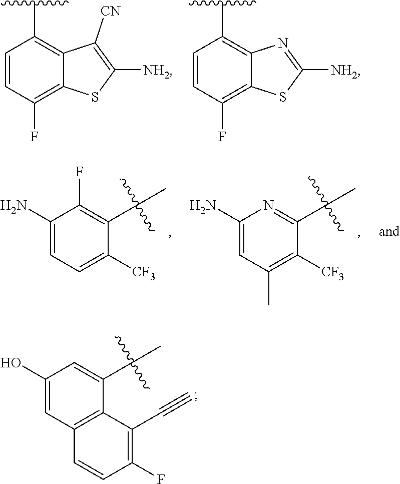 [SMILES: *c1ccc(F)c2sc(N)c(C#N)c12 ; *c1ccc(F)c2sc(N)nc12 ; *c1c(C(F)(F)F)ccc(N)c1F ; *c1nc(N)cc(C)c1C(F)(F)F ; *c1cc(O)cc2ccc(F)c(C#C)c12]

R¹⁶ is independently selected from hydrogen and halogen; R² is selected from

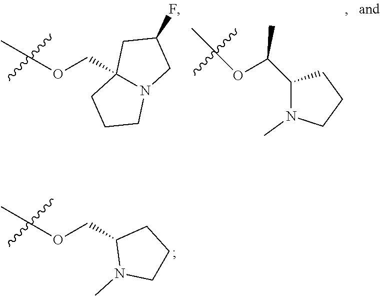

[symbol] indicates a single or double bond such that all valences are satisfied; and
all other variables of the formula (e.g., s1, s2, s3, R¹, R⁴, R⁶, R¹², R¹³, R¹⁴, R^{14a}, R¹⁵, R^{20a}, R²¹, R^{21b}, R²², R²³, R²⁴, and R²⁵) are as described for Formula (I-4), or embodiments thereof.

In an aspect is provided a compound of Formula (Ig), or a pharmaceutically acceptable salt or solvate thereof:

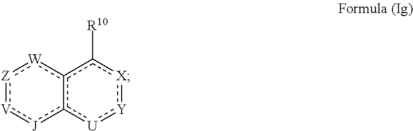

wherein
W is a CH or C(O); Z is N, C(R¹), or N(R^{1b}); V is C(R¹⁷); J is C(R¹⁶); Y is C(R²); U is N; X is N;
R¹⁰ is -L⁷-R⁷;
L⁷ is a bond;
R⁷ is selected from

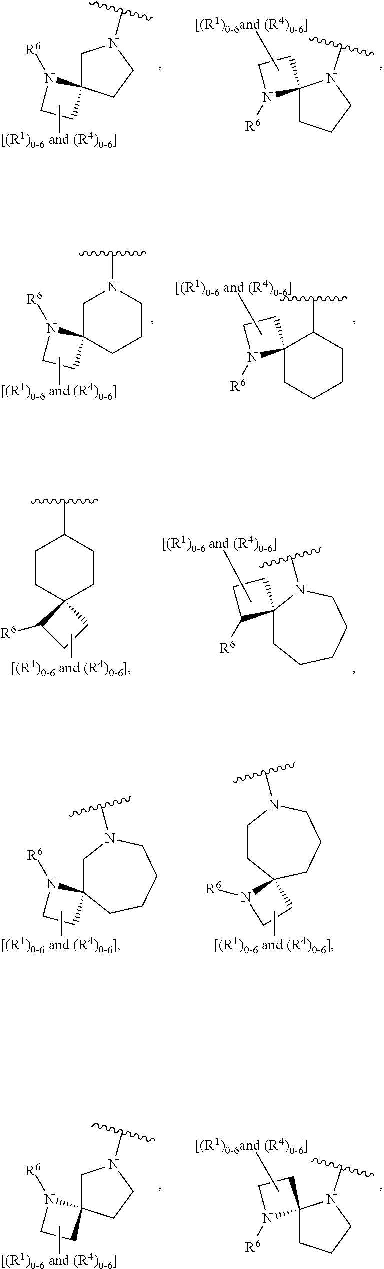

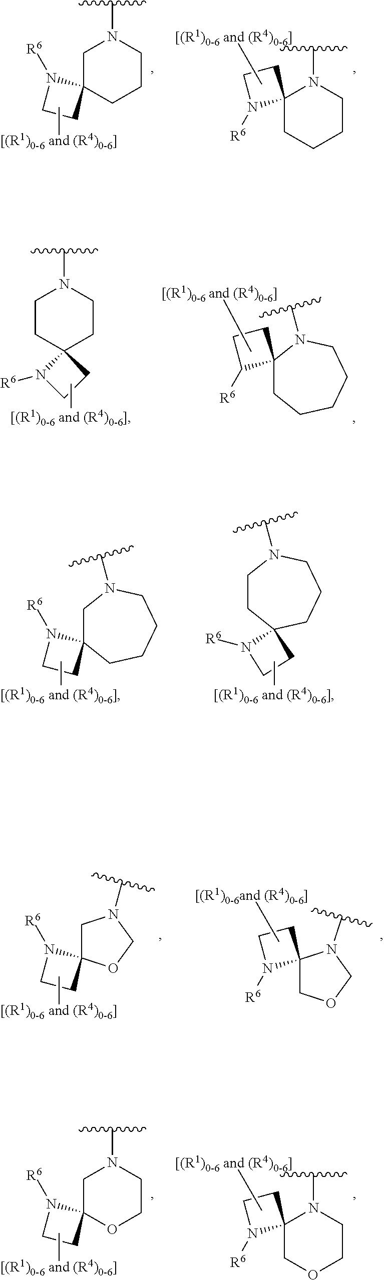

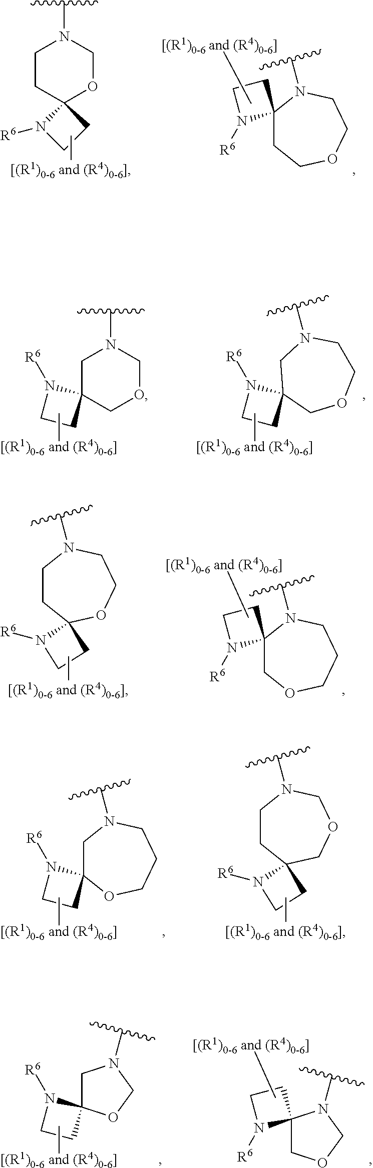

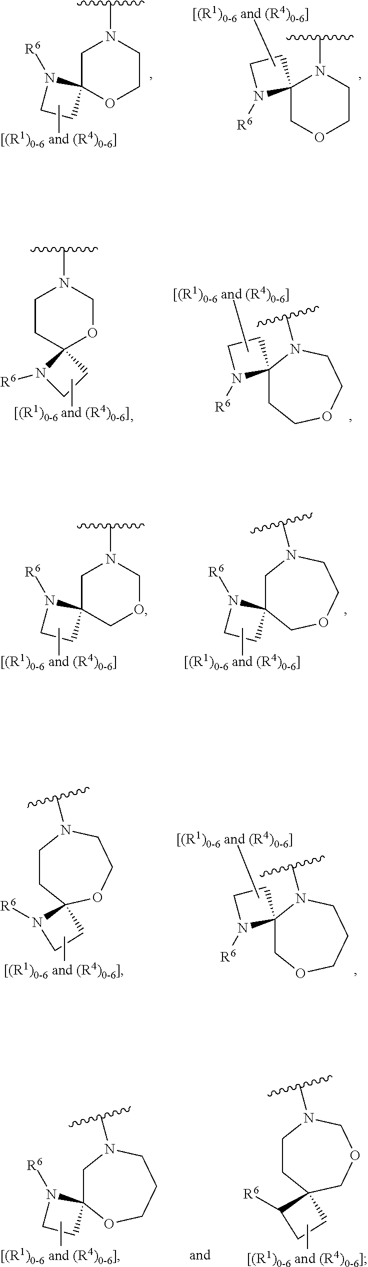

wherein R¹ and R⁴ substituents may be bonded to either spirocyclic ring;
L² is —C(O)—; R⁵ is a selected from

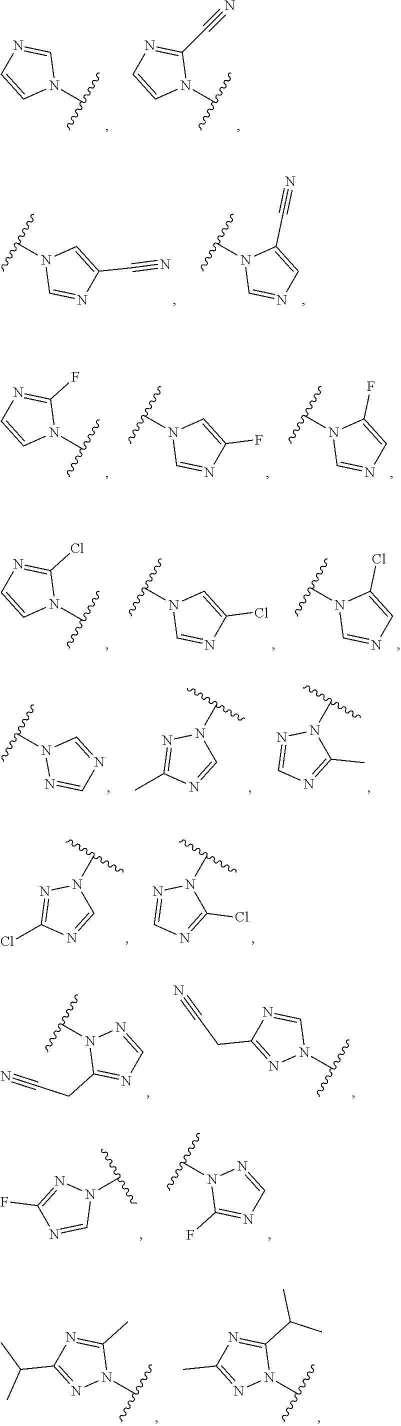

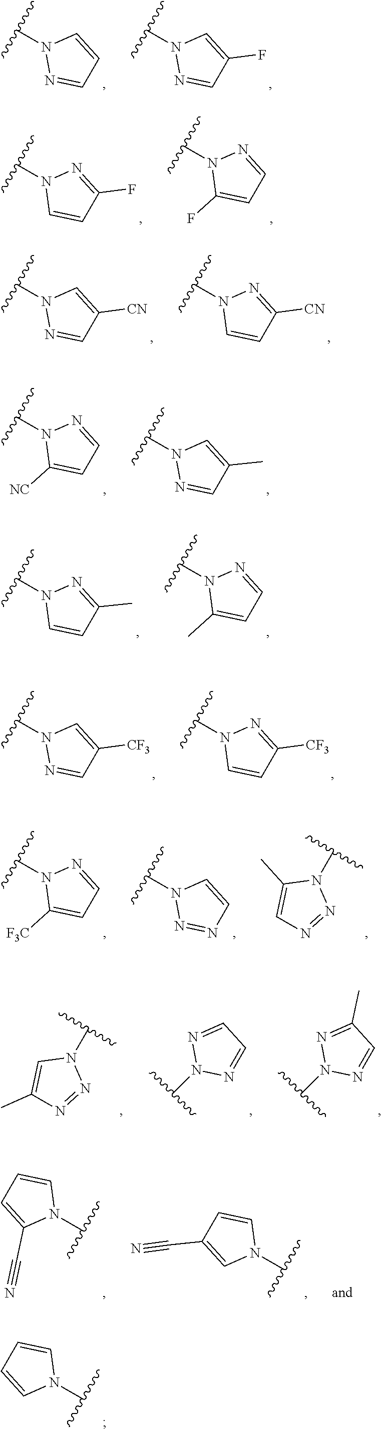

R⁸ is independently selected from hydrogen or halogen;
R^{8b} is selected from C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R¹⁷ is selected from

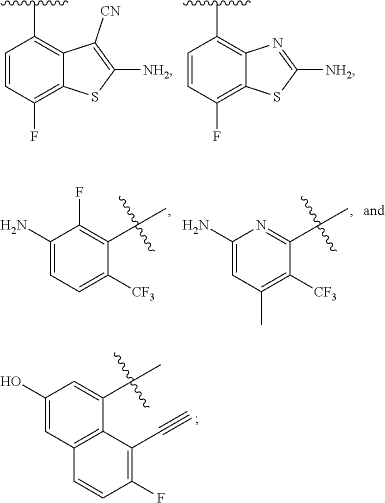

R¹⁶ is independently selected from hydrogen and halogen; R² is selected from

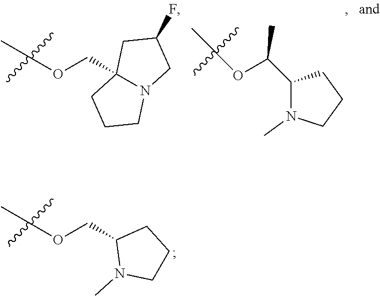

[symbol] indicates a single or double bond such that all valences are satisfied; and
all other variables of the formula (e.g., R¹, R⁴, R⁶, R¹², R¹³, R¹⁴, R^{14a}, R¹⁵, R^{20a}, R²⁰% R²¹, R^{21b}, R²², R²³, R²⁴, and R²⁵) are as described for Formula (I-4), or embodiments thereof.

In embodiments of Formula (Ig), [(R¹)₀₋₆ and (R⁴)₀₋₆] is equal to R⁴, and R⁴ is selected from halogen, methyl, and —CH₂CN. In embodiments of Formula (Ig), [(R¹)₀₋₆ and (R⁴)₀₋₆] is equal to [(R¹)o and (R⁴)O] (i.e., no R¹ or R⁴ substituent). In embodiments of Formula (Ig), [(R¹)₀₋₆ and (R⁴)₀₋₆] is equal to two R⁴, and each R⁴ is independently selected from halogen, methyl, and —CH₂CN.

In an aspect is provided a compound of Formula (Ig), or a pharmaceutically acceptable salt or solvate thereof:

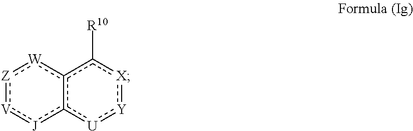

wherein
W is a CH or C(O); Z is N, C(R⁸), or N(R^{8b}); V is C(R¹⁷); J is C(R¹⁶); Y is C(R²); U is N; X is N;
R¹⁰ is -L⁷-R⁷;
L⁷ is a bond;
R⁷ is selected from

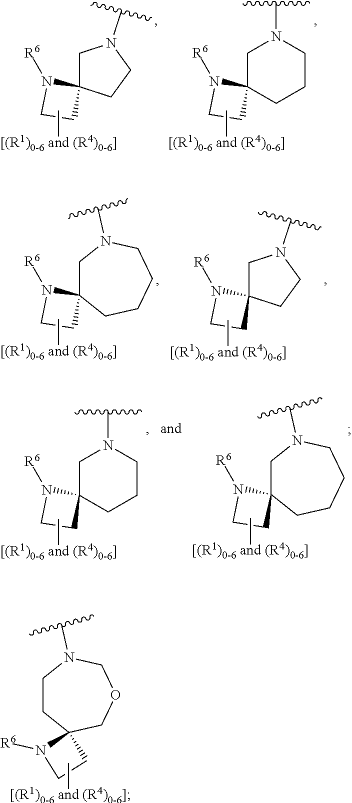

wherein R¹ and R⁴ substituents may be bonded to either spirocyclic ring;
L² is —C(O)—; R⁵ is a selected from

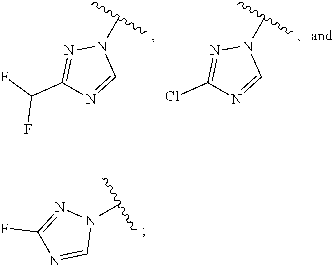 [SMILES: *n1cnc(C(F)F)n1 ; *n1cnc(Cl)n1 ; *n1cnc(F)n1]

R⁸ is independently selected from hydrogen or halogen;
R^{8b} is selected from C₁₋₂alkyl and C₃₋₄cycloalkyl, wherein C₁₋₂alkyl and C₃₋₄cycloalkyl are optionally substituted with one, two, or three R^{20c} selected from halogen and CN;
R¹⁷ is selected from

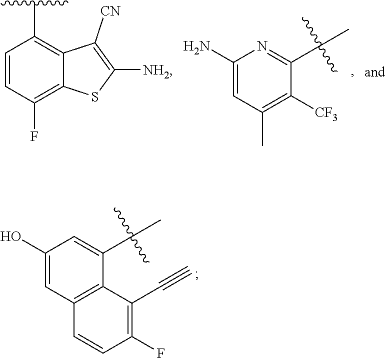 [SMILES: *c1ccc(F)c2sc(N)c(C#N)c12 ; *c1nc(N)cc(C)c1C(F)(F)F ; *c1cc(O)cc2ccc(F)c(C#C)c12]

R¹⁶ is independently selected from hydrogen and halogen; R² is selected from

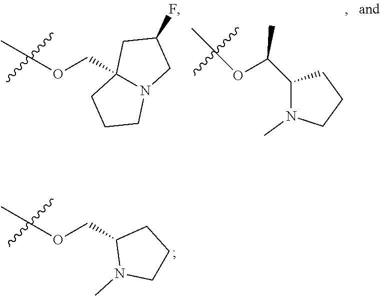

[symbol] indicates a single or double bond such that all valences are satisfied; and
all other variables of the formula (e.g., R¹, R⁴, R⁶, R¹², R¹³, R¹⁴, R^{14a}, R¹⁵, R^{20a}, R^{20c}, R²¹, R^{21b}, R²², R²³, R²⁴, and R²⁵) are as described for Formula (I-4), or embodiments thereof.

In an aspect is provided a compound of Formula (Ig), or a pharmaceutically acceptable salt or solvate thereof:

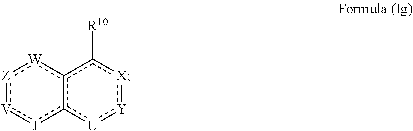

wherein
W is a CH or C(O); Z is N, C(R⁸), or N(R^{8b}); V is C(R¹⁷); J is C(R¹⁶); Y is C(R²); U is N; X is N;
R¹⁰ is -L⁷-R⁷; L⁷ is a bond;
R⁷ is

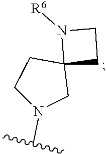

L² is —C(O)—; R⁵ is a selected from

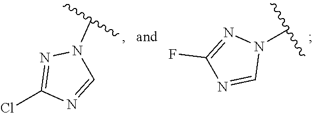 [SMILES: *n1cnc(Cl)n1 ; *n1cnc(F)n1]

R⁸ is independently —Cl; R^{8b} is cyclopropyl;
R¹⁷ is

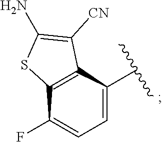

R¹⁶ is —F; R² is selected from

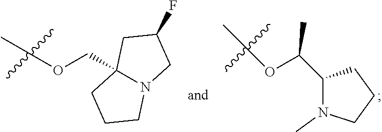

[symbol] indicates a single or double bond such that all valences are satisfied; and
all other variables of the formula (e.g., R¹, R⁴, R⁶, R¹², R¹³, R¹⁴, R^{14a}, R¹⁵, R^{20a}, R^{20c}, R²¹, R^{21b}, R²², R²³, R²⁴, and R²⁵) are as described for Formula (I-4), or embodiments thereof.

In embodiments of Formula (Ig), R² is

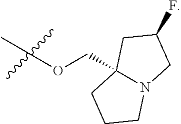 [SMILES: *OC[C@@]12CCCN1C[C@H](F)C2]

In embodiments of Formula (Ig), R² is

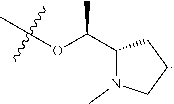 [SMILES: *O[C@@H](C)[C@@H]1CCCN1C]

In embodiments of Formula (Ig), R⁵ is

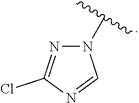 [SMILES: *n1cnc(Cl)n1]

In embodiments of Formula (Ig), R⁵ is

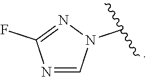 [SMILES: *n1cnc(F)n1]

In embodiments of Formula (Ig), W is a CH. In embodiments of Formula (Ig), W is C(O) and Z is N(R^{8b}). In embodiments of Formula (Ig), Z is N. In embodiments of Formula (Ig), Z is C(R⁸).

In an aspect is provided a compound of Formula (II-3), or a pharmaceutically acceptable salt or solvate thereof:

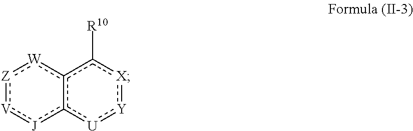

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
s1 is an integer from 1 to 6;
s2 is an integer from 0 to 3; and
all other variables of the formula (e.g., W, Z, V, J, U, Y, R¹⁰, L⁷, L², R⁷, R⁵, R¹⁹, R², W¹, W³, W⁵, s3, R¹, R⁴, R⁶, R⁸, R^{8a}, R^{8b}, R¹⁷, R^{17b}, L¹, L^{1b}, R^{1e}, R^{1f}, R^{1g}, R^{1c}, R¹ⁱ, R¹⁶, R^{16a}, R¹⁶, R^{2c}, R^{2b}, R^{12b}, X, R³, R¹², R^{12′}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (II), or embodiments thereof.

In an aspect is provided a compound of Formula (II′), or a pharmaceutically acceptable salt or solvate thereof:

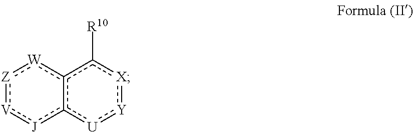

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
s1 is an integer from 1 to 6;
s2 is an integer from 0 to 3;
s3 is an integer from 1 to 3;
Y is N, C(R²), C(R²)(R^{2c}), N(R^{2b}), S(O), S(O)₂, or C(O);
R¹⁰ is -L⁷-R⁷;
R⁷ is

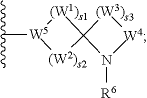

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R¹⁹ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl), —S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R¹ⁱ;
R² is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates the location of attachment of the depicted chemical formula or atom to a substituent, a further component of a molecule, or an atom;
[symbol] indicates a single or double bond such that all valences are satisfied; and
all other variables of the formula (e.g., W, Z, V, J, U, L⁷, W¹, W³, W⁵, R¹, R⁴, R⁶, R⁸, R^{8a}, R^{8b}, R¹⁷, R¹⁷, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″} R^{12c}, R¹³, R¹⁴, R^{14a}, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In an aspect is provided a compound of Formula (II″), or a pharmaceutically acceptable salt or solvate thereof:

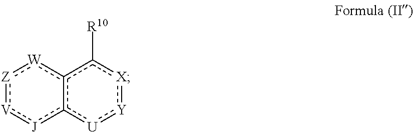

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
s1 is an integer from 1 to 6;
s2 is an integer from 0 to 3;
s3 is an integer from 1 to 3;
Y is N, C(R²), C(R²)(R^{2c}), N(R^{2b}), S(O), S(O)₂, or C(O);
R¹⁰ is -L⁷-R⁷;
R⁷ is

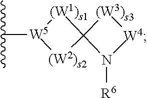

L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl), —S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R¹ⁱ;
R² is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, and —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates the location of attachment of the depicted chemical formula or atom to a substituent, a further component of a molecule, or an atom; and [symbol] indicates a single or double bond such that all valences are satisfied.
all other variables of the formula (e.g., W, Z, V, J, U, L⁷, W¹, W³, W⁵, R¹, R⁴, R⁶, R⁸, R^{8a}, R^{8b}, R¹⁷, R¹⁷, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In an aspect is provided a compound of Formula (IV-3), or a pharmaceutically acceptable salt or solvate thereof:

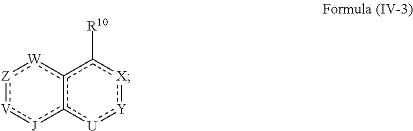

wherein
W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), and C(R⁴)(R⁴);
s1 is an integer from 1 to 6;
s2 is an integer from 0 to 3;
s1+s2 is not equal to 2 or 4;
R¹⁹ is selected from a C₂₋₆heterocycloalkyl and C₂₋₁₂heteroaryl, wherein the C₂₋₆heterocycloalkyl and C₂₋₁₂heteroaryl are optionally substituted with one, two, three, four, five, six, or seven R¹ⁱ;
R² is hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³), —S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₆heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d}; and
all other variables of the formula (e.g., W, Z, V, J, Y, U, R¹⁰, L⁷, R⁷, W¹, W³, W⁵, s3, R¹, R⁶, L², R⁴, R⁵, R^{4c}, R^{4d}, R⁸, R^{8a}, R^{8b}, R¹⁷, R^{17b}, L¹, L^{1b}, R^{1e}, R¹, R^{1a}, R^{1b}, R^{1c}, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R²², R²³, R²⁴, and R²⁵) are as described for Formula (IV), or embodiments thereof.

In an aspect is provided a compound of Formula (IV-4), or a pharmaceutically acceptable salt or solvate thereof:

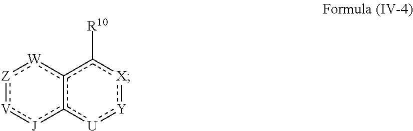

wherein
Y is N, C(R²), C(R²)(R^{2c}), N(R^{2b}), S(O), S(O)₂, or C(O);
R¹⁰ is -L⁷-R⁷;
R⁷ is

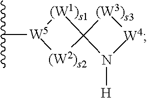

W² is independently selected from a N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), or C(R⁴)(R⁴), s1 is an integer from 1 to 6 and s2 is an integer selected from 2 and 3; or s1 is an integer from 2 to 6 and s2 is an integer from 1 to 3;
s1+s2 is not equal to 2 or 4;
R¹⁹ is selected from a C₂₋₁₁heterocycloalkyl and C₂₋₁₂heteroaryl, wherein the C₂₋₁₂heterocycloalkyl and C₂₋₁₂heteroaryl are optionally substituted with one, two, three, four, five, six, or seven R¹ⁱ;
R² is hydrogen, halogen, —CN, C₁₋₆alkyl, C₂₋₆alkenyl, C₂₋₆alkynyl, C₃₋₁₀cycloalkyl, C₄₋₉heterocycloalkyl, C₆₋₁₀aryl, C₁₋₉heteroaryl, —OR^{12′}, —SR^{12′}, —N(R^{12′})(R¹³), —N═(R¹⁵), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₄₋₉ heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
[symbol] indicates a single or double bond such that all valences are satisfied; and
all other variables of the formula (e.g., W, Z, V, J, U, L⁷, W¹, W³, W⁵, s3, R¹, R⁴, R⁸, R^{8a}, R^{8b}, R¹⁷, R¹⁷, L¹, L^{1b}, R^{1e}, R^{1f}, R^{1g}, R^{1c}, R¹ⁱ, R¹⁶, R^{16a}, R¹⁶, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20d}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), or embodiments thereof.

In an aspect is provided a compound of Formula (I), or a pharmaceutically acceptable salt or solvate thereof:

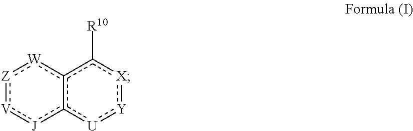

wherein
W is a C(R¹⁸) or C(O); Z is N, C(R⁸), or N(R^{8b}); V is C(R¹⁷); J is C(R¹⁶); Y is C(R²); U is N; X is N;
R¹⁰ is -L⁷-R⁷;
L⁷ is a bond;
R⁷ is

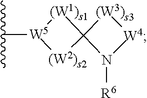

W¹, W², and W³ are independently selected from C(R¹)(R¹), C(R¹)(R⁴), C(R⁴)(R⁴), and O;
W⁴ is selected from C(R¹)(R¹), C(R¹)(R⁴), and C(R⁴)(R⁴);
W⁵ is selected from N and CH;
s1 is an integer from 1 to 6;
s2 is an integer from 1 to 3;
s3 is an integer from 1 to 3;
R⁶ is -L²-R⁵ and wherein R⁶ is optionally capable of forming a covalent bond with a Ras amino acid;
L² is selected from —C(O)—;
R⁵ is a 5-6 membered heteroaryl, optionally substituted with one, two, or three R^{20k}, wherein the 5-6 membered heteroaryl comprises one, two, or three ring nitrogen atoms and further wherein when R⁵ is directly bonded to a C(O) of L², L² is directly bonded to an N atom of R⁵;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), ═C(R^{21b})₂, —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹², —S(O)₂R¹⁵, —S(O)₂N(R¹²)(R¹³)—, S(═O)(═NH)N(R¹²)(R¹³), —(C₁₋₆alkyl)-C(O)N(R¹²)(R¹³), —(C₁₋₆alkyl)-N(R¹⁴)C(O)R¹², —(C₁₋₆alkyl)-S(O)₂R¹⁵, —(C₁₋₆alkyl)-N(R¹²)(R¹³), and —(C₁₋₆alkyl)-S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁸ is independently selected from hydrogen, halogen, C₁₋₆alkyl, C₂₋₆alkenyl, C₂₋₆alkynyl, C₃₋₁₀cycloalkyl, C₂₋₉heterocycloalkyl, —OR¹², —SR¹², and —N(R¹²)(R¹³), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R^{8b} is selected from hydrogen, C₁₋₆alkyl, C₂₋₆alkenyl, C₂₋₆alkynyl, C₃₋₁₀cycloalkyl, and C₂₋₉heterocycloalkyl, wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R¹⁷ is -L¹-R¹⁹;
L¹ is a bond;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ;
R¹⁶ is independently selected from hydrogen, halogen, C₁₋₆alkyl, C₂₋₆alkenyl, C₂₋₆alkynyl, C₃₋₁₀cycloalkyl, C₂₋₉heterocycloalkyl, —OR¹², —SR¹², and —N(R¹²)(R¹³), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20g};
R² is selected from C₁₋₆alkyl, C₂₋₆alkenyl, C₂₋₆alkynyl, C₃₋₁₀cycloalkyl, C₂₋₉heterocycloalkyl, —OR^{12′}, —SR^{12′}, and —N(R^{12″})(R¹³), wherein said C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20d};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{2d};
each R^{12″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f}, R¹⁸ is selected from hydrogen, halogen, C₁₋₆alkyl, C₂₋₆alkenyl, C₂₋₆alkynyl, C₃₋₁₀cycloalkyl, and C₂₋₉heterocycloalkyl, wherein C₁₋₆ alkyl, C₂₋₆alkenyl, C₂₋₆alkynyl, C₃₋₁₀cycloalkyl, and C₂₋₉heterocycloalkyl are optionally substituted with one, two, or three R^{20h};
each R^{20a}, R^{20b}, R^{2c}, R^{20d}, R²⁰, R^{20f}, R^{20g}, R^{20c}, R²⁰ⁱ, W^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), ═C(R^{21b})₂, —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²¹, —N(R²⁴)S(O)₂R²⁵, —C(O)R²¹, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
R^{21b} is independently selected at each occurrence from hydrogen, halogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, or two R^{21b} are taken together with the carbon atom to which they are attached to form C₃₋₁₀cycloalkyl or C₂₋₉heterocycloalkyl; each of which is optionally substituted with one, two, or three substituents independently selected from halogen, C₁₋₃ alkyl, C₁₋₃ haloalkyl, and —OH;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl; and
[symbol] indicates a single or double bond such that all valences are satisfied.
   In embodiments of Formula (I), R⁷ is selected from

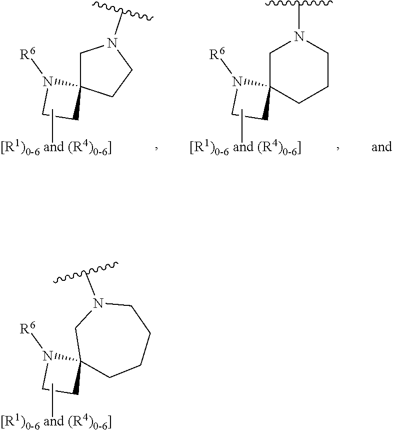

wherein R¹ and R⁴ substituents may be bonded to either spirocyclic ring; R¹⁷ is selected from

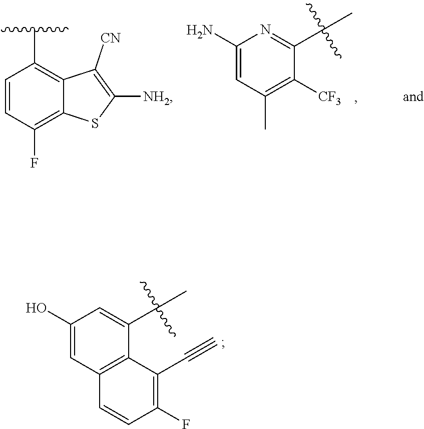 [SMILES: *c1ccc(F)c2sc(N)c(C#N)c12 ; *c1nc(N)cc(C)c1C(F)(F)F ; "and" ; *c1cc(O)cc2ccc(F)c(C#C)c12]

and R² is selected from

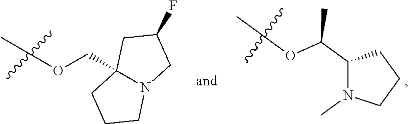

In embodiments, the compound of Formula (I) is a compound of the formula

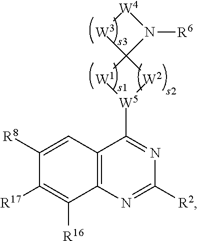

or a pharmaceutically acceptable salt or solvate thereof.

In embodiments of Formula (I), (I′), (I-3), (I-4), (II-3), (IV-3), (IV-4), (Ia), (Ib), (Ic), (Id), (Ie), (If), (Ig), (II), (II′), (II″), (IV-3), (IV-4), (IV), (IV′), or (IVa), the compound has a formula selected from:

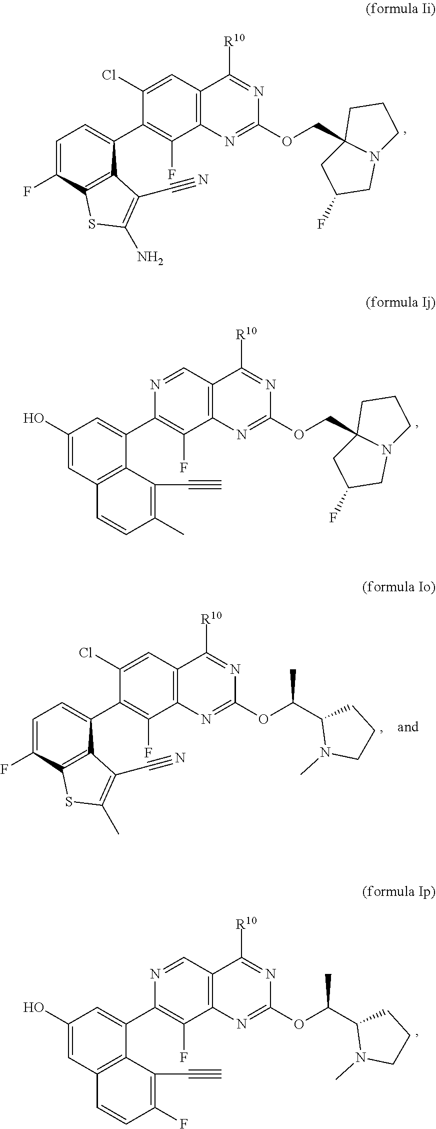

wherein R¹⁰ is as described herein, including in any aspect or embodiment.

In embodiments of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″),
R⁷ is

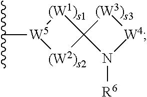

at least one W³ and/or W⁴ is C(R¹)(R¹), C(R¹)(R⁴), or C(R⁴)(R⁴).
R¹ and R⁴ are as described for Formula (I-4), including in embodiments of a compound of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″); and at least one i) R¹ of W³ or W⁴ is not hydrogen or ii) one R⁴ of W³ or W⁴ is not hydrogen.

In embodiments of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″),
R⁷ is

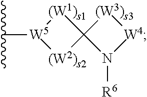

at least one W³ and/or W⁴ is C(R¹)(R¹), C(R¹)(R⁴), or C(R⁴)(R⁴).
R¹ and R⁴ are as described for Formula (I-4), including in embodiments of a compound of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″). In embodiments, R¹ and R⁴ are hydrogen.

In embodiments of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″),
R⁷ is

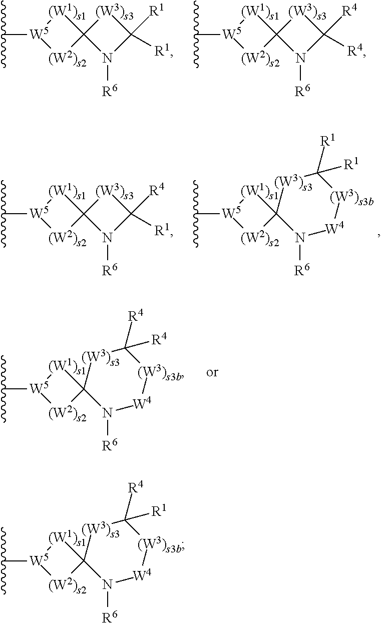

R¹ and R⁴ are as described for Formula (I-4), including in embodiments of a compound of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″);
at least one R¹ or R⁴ shown in the formulae immediately above is not hydrogen; and
s3a is 0, 1, or 2; s3b is 0, 1, or 2; the sum of s3a and s3b is 0, 1, or 2.

In embodiments of Formula (IV), (IV-3), (IV-4), (IVa), or (IV′),
R⁷ is

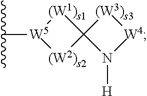

at least one W³ and/or W⁴ is C(R¹)(R¹), C(R¹)(R⁴), or C(R⁴)(R⁴).
R¹ and R⁴ are as described for Formula (I-4), including in embodiments of a compound of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″); and
at least one i) R¹ of W³ or W⁴ is not hydrogen or ii) one R⁴ of W³ or W⁴ is not hydrogen.

In embodiments of Formula (IV), (IV-3), (IV-4), (IVa), or (IV′),
R⁷ is

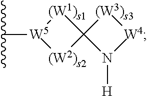

at least one W³ and/or W⁴ is C(R¹)(R¹), C(R¹)(R⁴), or C(R⁴)(R⁴).
R¹ and R⁴ are as described for Formula (I-4), including in embodiments of a compound of Formula (I), (I′), (I-3), (I-4), (II-3), (Ia), (Ib), (Ic), (Id), (Ie), (If), (Ii), (Ij), (Ik), (Im), (In), (Io), (Ip), (Iq), (Ir), (II), (II′), or (II″). In embodiments, R¹ and R⁴ are hydrogen.

In embodiments, the compound has the formula

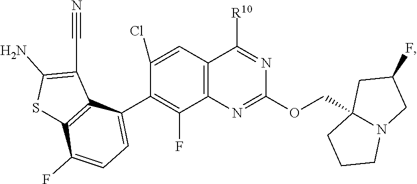

wherein R¹⁰ is

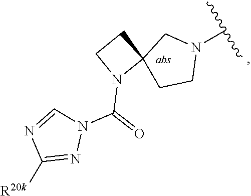

and R^{20k} is selected from halogen, C₁₋₃alkyl, and C₁₋₂haloalkyl. In embodiments, the compound has the formula

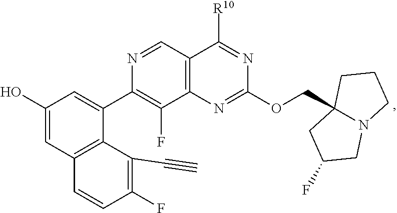

wherein R¹⁰ is

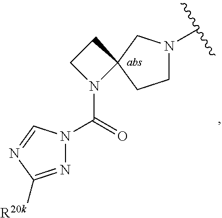

and R^{20k} is selected from halogen, C₁₋₃alkyl, and C₁₋₂haloalkyl. In embodiments, the compound has the formula

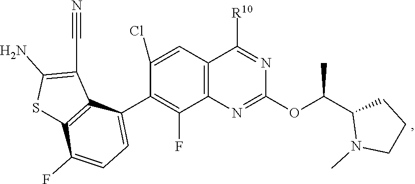

wherein R¹⁰ is

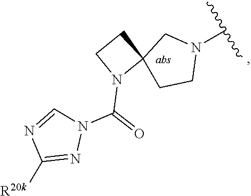

and R^{20k} is selected from halogen, C₁₋₃alkyl, and C₁₋₂haloalkyl. In embodiments, the compound has the formula

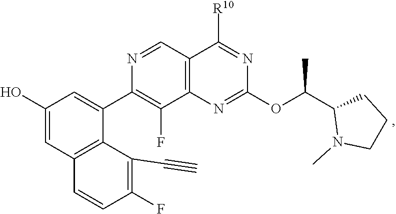

and W^{20k} is selected from

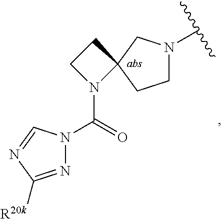

halogen, C₁₋₃alkyl, and C₁₋₂ haloalkyl. In embodiments of any one of the formulae above, R^{20k} is halogen. In embodiments of any one of the formulae above, R^{20k} is Cl. In embodiments of any one of the formulae above, W^{20k} is F. In embodiments of any one of the formulae above, R^{20k} is —CHF₂. In embodiments of any one of the formulae above, R^{20k} is —CF₃.

In embodiments, R¹⁰ is

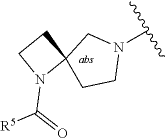

and R⁵ is selected from

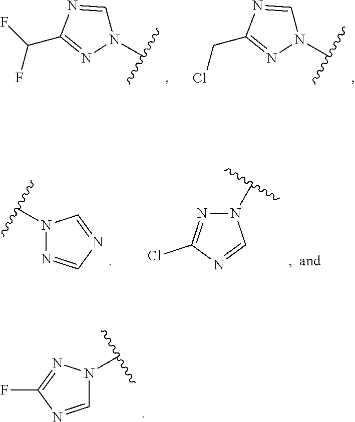 [SMILES: *n1cnc(C(F)F)n1 ; *n1cnc(CCl)n1 ; *n1cncn1 ; *n1cnc(Cl)n1 ; *n1cnc(F)n1]

In embodiments, R¹⁰ is

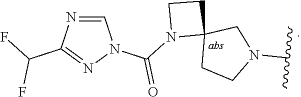 [SMILES: *N1CC[C@@]2(CCN2C(=O)n2cnc(C(F)F)n2)C1]

In embodiments, R¹⁰ is

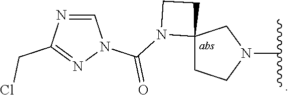 [SMILES: *N1CC[C@@]2(CCN2C(=O)n2cnc(CCl)n2)C1]

In embodiments, R¹⁰ is

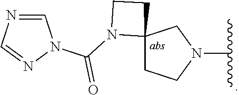 [SMILES: *N1CC[C@@]2(CCN2C(=O)n2cncn2)C1]

In embodiments, R¹⁰ is

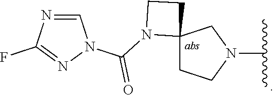 [SMILES: *N1CC[C@@]2(CCN2C(=O)n2cnc(F)n2)C1]

In embodiments, R¹⁰ is

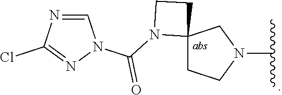 [SMILES: *N1CC[C@@]2(CCN2C(=O)n2cnc(Cl)n2)C1]

In embodiments, R¹⁰ is

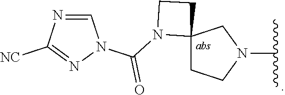 [SMILES: *N1CC[C@@]2(CCN2C(=O)n2cnc(C#N)n2)C1]

In embodiments, R¹⁰ is

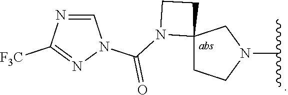 [SMILES: *N1CC[C@@]2(CCN2C(=O)n2cnc(C(F)(F)F)n2)C1]

In embodiments, R¹⁰ is

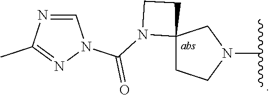 [SMILES: *N1CC[C@@]2(CCN2C(=O)n2cnc(C)n2)C1]

In embodiments, R¹⁰ is

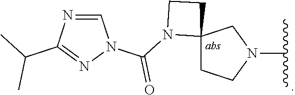 [SMILES: *N1CC[C@@]2(CCN2C(=O)n2cnc(C(C)C)n2)C1]

In an aspect is provided a compound having the formula:

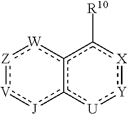

wherein
R¹⁹ is selected from

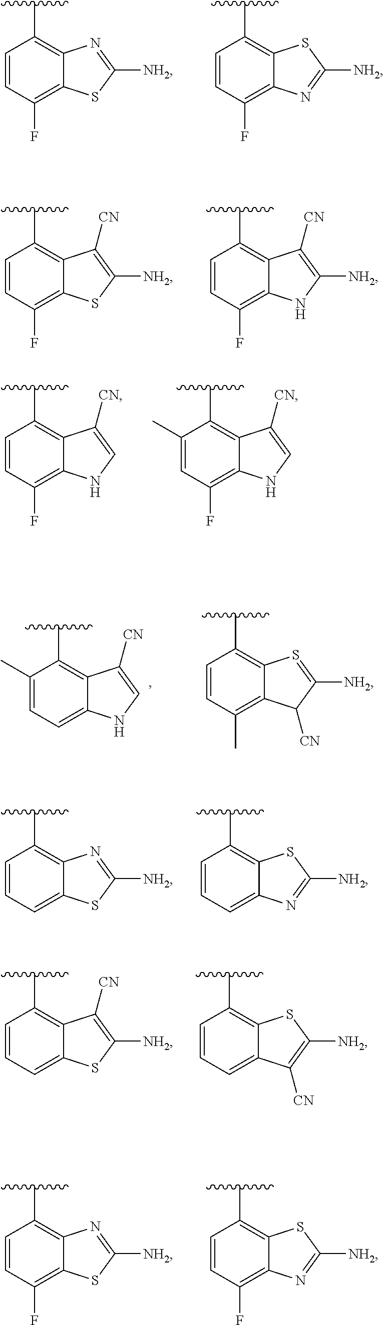

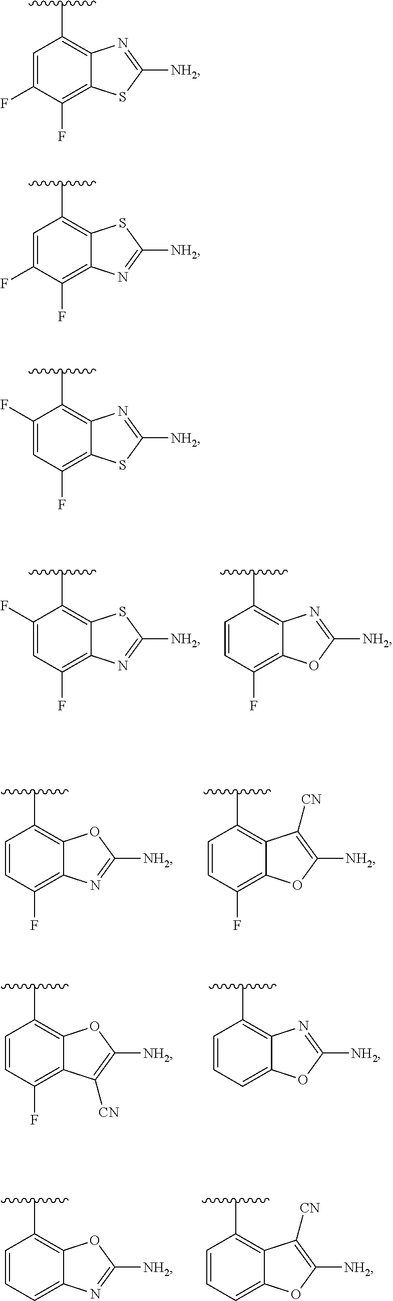

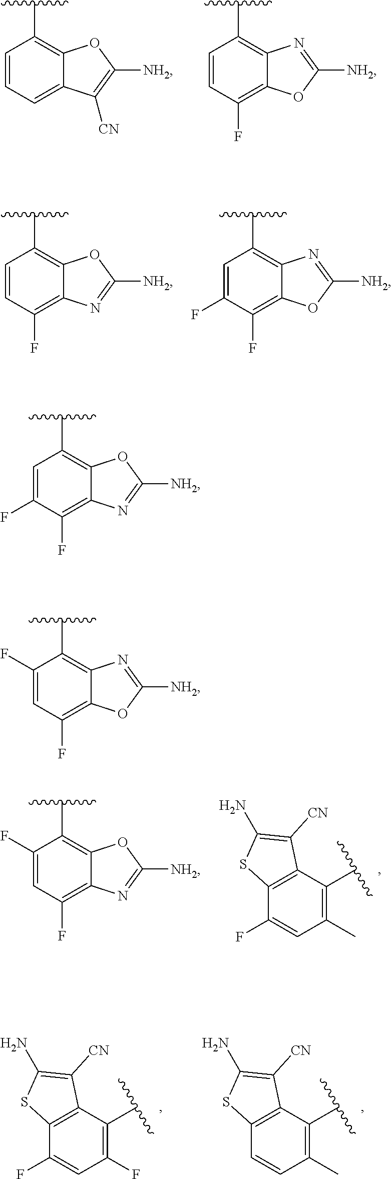

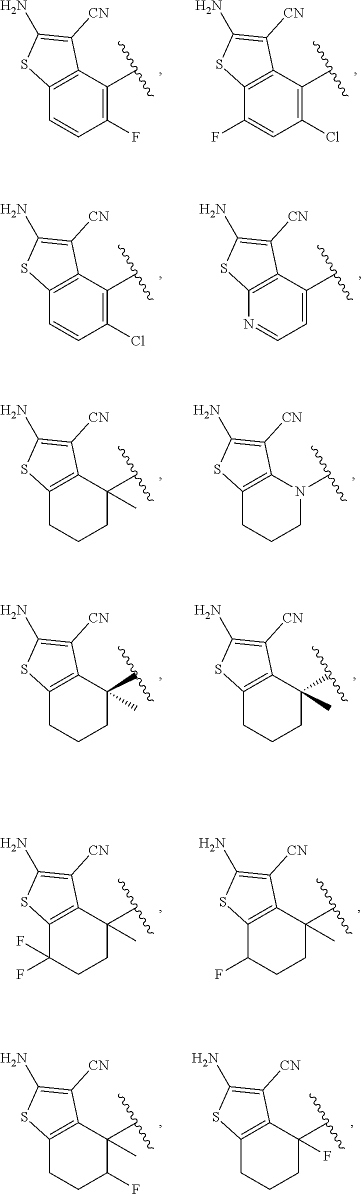

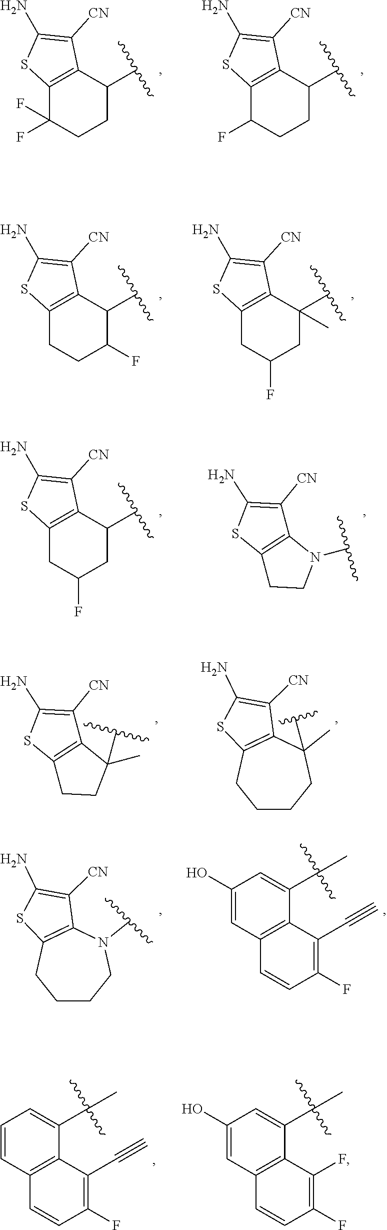

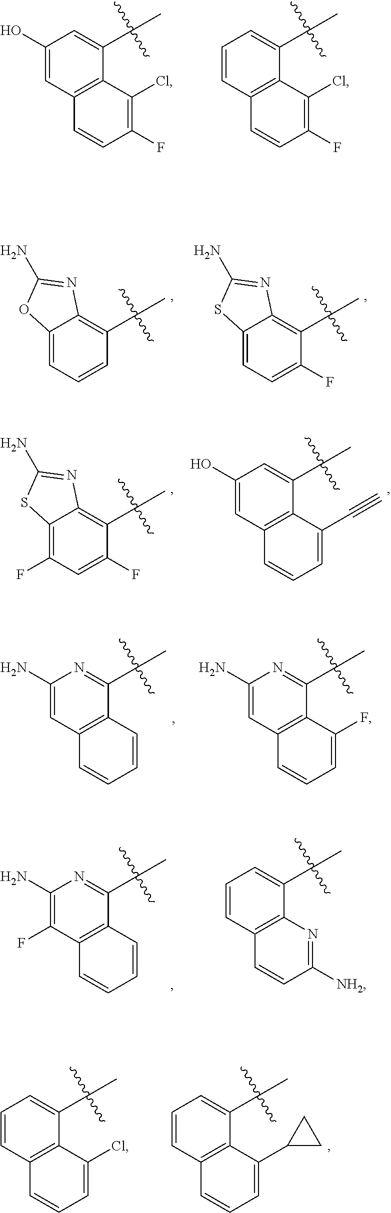

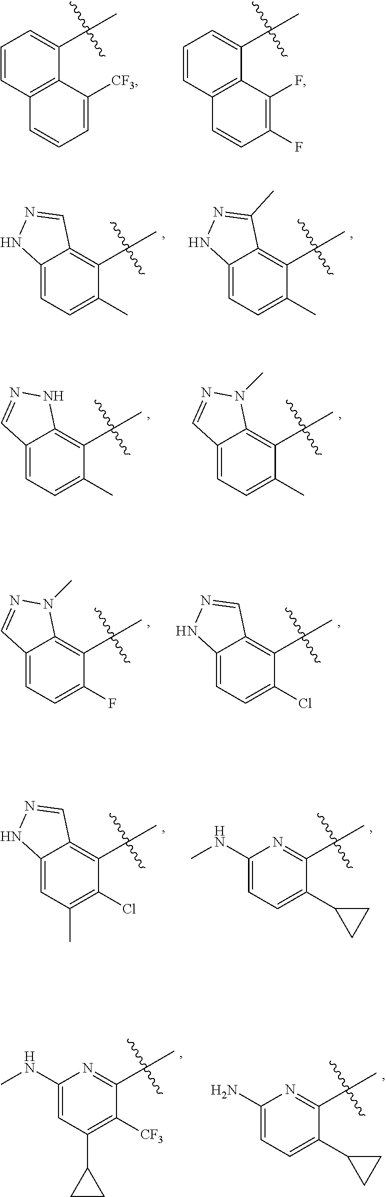

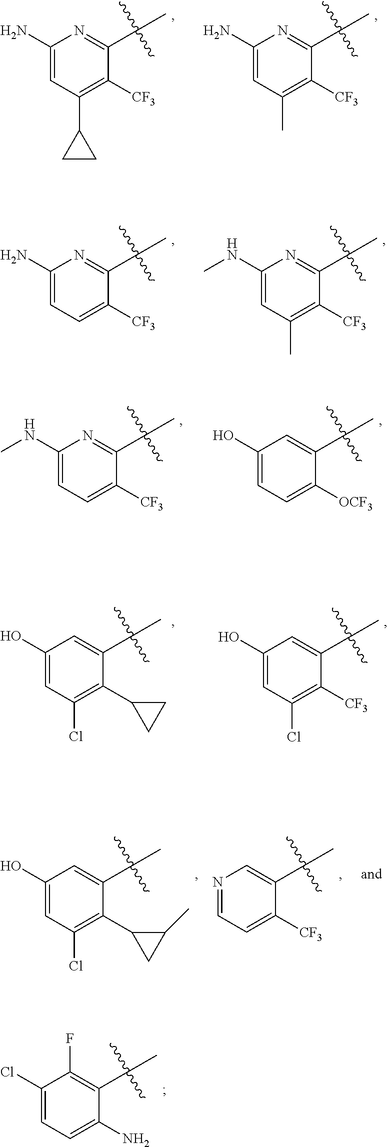

[symbol] indicates a single or double bond such that all valences are satisfied; and
all other variables of the formula (e.g., W, Z, V, J, Y, U, R¹⁰, L⁷, R⁷, W¹, W³, W², W⁴, W⁵, s1, s2, s3, R¹, R², R⁴, R⁵, R⁶, L², R⁴, R⁴, R, R^{8a}, R^{8b}, R¹⁷, R^{17b}, L¹, L^{1b}, R^{1e}, R^{1f}, R^{1g}, R¹, R¹ⁱ, R¹⁶, R^{16a}, R^{16b}, R^{2c}, R^{2b}, R^{12b}, X, R³, R¹², R^{12′}, R^{12″}, R^{12c}, R¹³, R¹⁴, R^{14a}, R¹⁵, R¹⁸, R^{18a}, R^{18b}, R^{20a}, R^{20b}, R^{20c}, R^{20a}, R^{20e}, R^{20f}, R^{20g}, R^{20h}, R²⁰ⁱ, R^{20k}, R^{20m}, R^{21a}, R^{21b}, R²², R²³, R²⁴, and R²⁵) are as described for Formula (I-4), (II-3), or (IV-4), or embodiments thereof.

In an aspect is provided a compound having the formula:

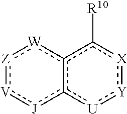

wherein
V and J are each independently selected from C(R¹⁷), C(O), C(R¹⁷)(R^{16a}), C(R¹⁶), C(R¹⁶)(R^{16a}), N, N(R¹⁷), and N(R¹⁶); wherein exactly one of V and J is C(R¹⁷), C(R¹⁷)(R^{16a}), or N(R¹⁷);
X is C(O), C(R³), C(R³)(R³), N(R³), or N;
[symbol] indicates a single or double bond such that all valences are satisfied; and
all other variables of the formula (e.g., W, Z, Y, U, R¹¹, L⁷, R⁷, W¹, W³, W², W⁴, W⁵, s1, s2, s3, R¹, R², R⁴, R⁵, R⁶, L², R^{4c}, R^{4d}, R⁸, R^{8a}, R⁸, R¹⁷, R¹⁷, L¹, L^{1b}, R^{1e}, R^{1f}, R^{1g}, R^{1c}, R¹ⁱ, R¹⁶, R^{16a}, R^{16b}, R^{2c}, R^{2b}, R^{12b}, R³, R¹², R^{12′}, R^{12″}, R^{12c}, R¹³, R¹⁴, R^{14a}, R¹⁵, R¹⁸, R^{18a}, R^{18b}, R¹⁹, R^{20a}, R^{20b}, R^{20c}, R^{20a}, R^{20e}, R^{20f}, R^{20g}, R^{20h}, R²⁰ⁱ, R^{20k}, R^{20m}, R²¹, R^{21b}, R²², R²³, R²⁴, and R²⁵) are as described for Formula (I-4), (II-3), or (IV-4), or embodiments thereof.

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

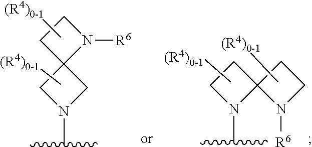

wherein each R⁴ substituent is attached to an atom of only the ring on which the R⁴ substituent is floating; and wherein R⁷ is substituted with one or two R⁴; and each R⁴ is independently selected from halogen, —CN, C₁₋₄alkyl, and C₃₋₄cycloalkyl, wherein C₁₋₄alkyl and C₃₋₄cycloalkyl are optionally substituted with one, two, or three R^{20a} selected from halogen, —CN, —OH, and —NH₂. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

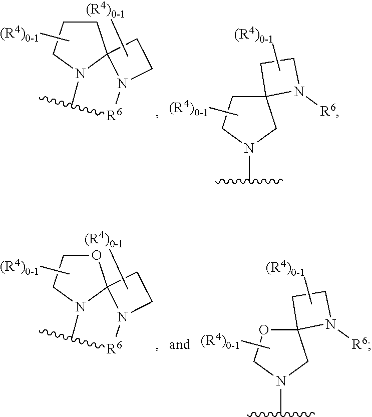

wherein each R⁴ substituent is attached to an atom of only the ring on which the R⁴ substituent is floating; and wherein R⁷ is substituted with one or two R⁴; and each R⁴ is independently selected from halogen, —CN, C₁₋₄alkyl, and C₃₋₄cycloalkyl, wherein C₁₋₄alkyl and C₃₋₄cycloalkyl are optionally substituted with one, two, or three R^{20a} selected from halogen, —CN, —OH, and —NH₂. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

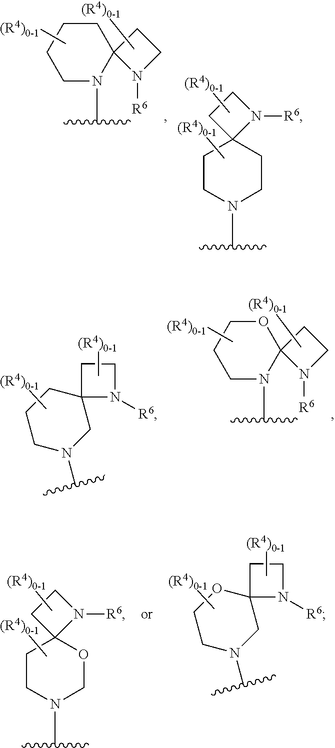

wherein each R⁴ substituent is attached to an atom of only the ring on which the R⁴ substituent is floating; and wherein R⁷ is substituted with one or two R⁴; and each R⁴ is independently selected from halogen, —CN, C₁₋₄alkyl, and C₃₋₄cycloalkyl, wherein C₁₋₄alkyl and C₃₋₄cycloalkyl are optionally substituted with one, two, or three R^{20a} selected from halogen, —CN, —OH, and —NH₂. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

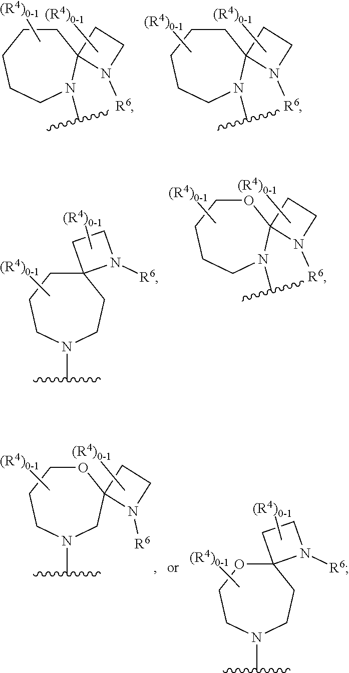

wherein each R⁴ substituent is attached to an atom of only the ring on which the R⁴ substituent is floating; and wherein R⁷ is substituted with one or two R⁴; and each R⁴ is independently selected from halogen, —CN, C₁₋₄alkyl, and C₃₋₄cycloalkyl, wherein C₁₋₄alkyl and C₃₋₄cycloalkyl are optionally substituted with one, two, or three R^{20a} selected from halogen, —CN, —OH, and —NH₂. In embodiments, R⁷ is a single spirocyclic R stereoisomer. In embodiments, R⁷ is a single spirocyclic S stereoisomer. In embodiments, R⁷ is substituted with one R⁴ substituent wherein the carbon atom attached to the R⁴ substituent is an R isomer. In embodiments, R⁷ is substituted with one R⁴ substituent wherein the carbon atom attached to the R⁴ substituent is an S isomer. In embodiments, R⁷ is substituted with two optionally different R⁴ substituents wherein the carbon atoms attached to the R⁴ substituents are both R isomers. In embodiments, R⁷ is substituted with two optionally different R⁴ substituents wherein the carbon atoms attached to the R⁴ substituents are both S isomers. In embodiments, R⁷ is substituted with two optionally different R⁴ substituents wherein the carbon atom attached to the R⁴ substituent in the ring bonded directly to R⁶ is an R isomer and the carbon atom attached to the R⁴ substituent in the other ring is an S isomer. In embodiments, R⁷ is substituted with two optionally different R⁴ substituents wherein the carbon atom attached to the R⁴ substituent in the ring bonded directly to R⁶ is an S isomer and the carbon atom attached to the R⁴ substituent in the other ring is an R isomer. In embodiments, each R⁴ is independently selected from F, —CN, methyl, ethyl, isopropyl, cyclopropyl, cyclobutyl, —CHF₂,

In embodiments of Formula (IV-3), (IV-4), (IV), (IVa), or (IV′), R⁷ is not

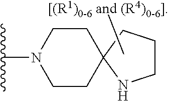

In embodiments of Formula (IV-3), (IV-4), (IV), (IVa), or (IV′), R⁷ is not

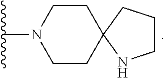 [SMILES: *N1CCC2(CCCN2)CC1]

In embodiments of Formula (IV-3), (IV-4), (IV), (IVa), or (IV′), R¹⁰ is not

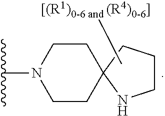

In embodiments of Formula (IV-3), (IV-4), (IV), (IVa), or (IV′), R¹⁰ is not

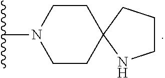 [SMILES: *N1CCC2(CCCN2)CC1]

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is not

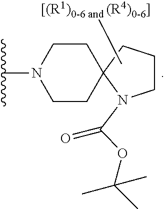

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is not

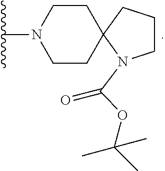 [SMILES: *N1CCC2(CCCN2C(=O)OC(C)(C)C)CC1]

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is not

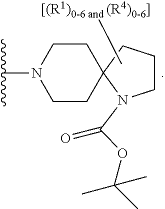

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is not

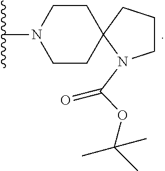 [SMILES: *N1CCC2(CCCN2C(=O)OC(C)(C)C)CC1]

In embodiments of Formula (L), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is not

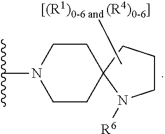

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is not

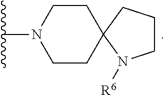

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is not

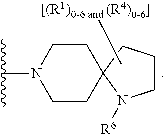

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is not

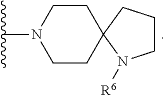

The individual embodiments herein below, or combinations thereof, (e.g., embodiments of R⁶) are applicable to compounds of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), or a pharmaceutically acceptable salt or solvate thereof. In embodiments of the formulae above, R⁶ is capable of forming a covalent bond with a Ras amino acid sidechain. In embodiments of the formulae above, R⁶ is capable of forming a covalent bond with a KRas amino acid. In embodiments of the formulae above, R⁶ is capable of forming a covalent bond with the 12^{th} amino acid of a human KRas protein. In embodiments of the formulae above, R⁶ is capable of forming a covalent bond with the 12^{th} amino acid of a mutant KRas protein selected from KRas G12D, KRas G12C, and KRas G12S. In embodiments of the formulae above, R⁶ is capable of forming a covalent bond with the 13^{th} amino acid of a human KRas protein. In embodiments of the formulae above, R⁶ is capable of forming a covalent bond with the 13^{th} amino acid of a mutant KRas protein selected from KRas G13D, KRas G13C, and KRas G13S.

In embodiments of the formulae above, R⁶ is incapable of forming a covalent bond with a Ras amino acid sidechain. In embodiments of the formulae above, R⁶ is incapable of forming a covalent bond with a KRas amino acid. In embodiments of the formulae above, R⁶ is incapable of forming a covalent bond with the 12^{th} amino acid of a human KRas protein. In embodiments of the formulae above, R⁶ is incapable of forming a covalent bond with the 12^{th} amino acid of a mutant KRas protein selected from KRas G12D, KRas G12C, and KRas G12S. In embodiments of the formulae above, R⁶ is incapable of forming a covalent bond with the 13^{th} amino acid of a human KRas protein. In embodiments of the formulae above, R⁶ is incapable of forming a covalent bond with the 13^{th} amino acid of a mutant KRas protein selected from KRas G13D, KRas G13C, and KRas G13S.

In embodiments of the formulae above, R⁶ is incapable of forming a covalent bond with the 121 amino acid of a human KRas protein selected from KRas wildtype, KRas G12D, KRas G12C, KRas G12S, KRas G12V, KRas G13D, KRas G13C, KRas G13S, and KRas G13V. In embodiments of the formulae above, R⁶ is incapable of forming a covalent bond with the 13^{th} amino acid of human KRas protein selected from KRas wildtype, KRas G12D, KRas G12C, KRas G12S, KRas G12V, KRas G13D, KRas G13C, KRas G13S, and KRas G13V.

In embodiments of Formula (I), (I-3), (I-4), (Ia), (Ib), (Ic), (Id), (Ii), (Ij), (Ik), (Im), (In), (Io), (Ip), (Iq), (Ir), or (I′), R⁶ is selected from the group consisting of

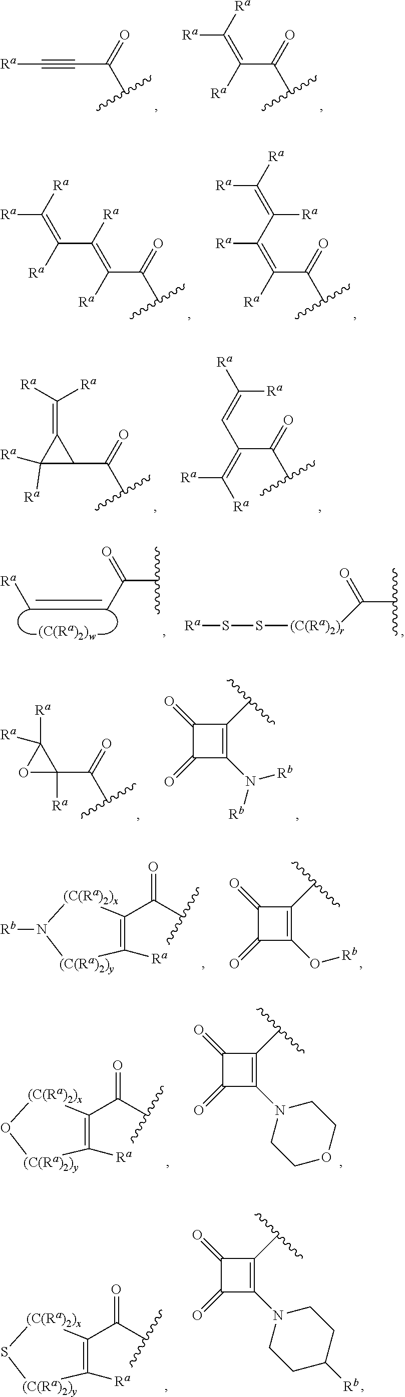

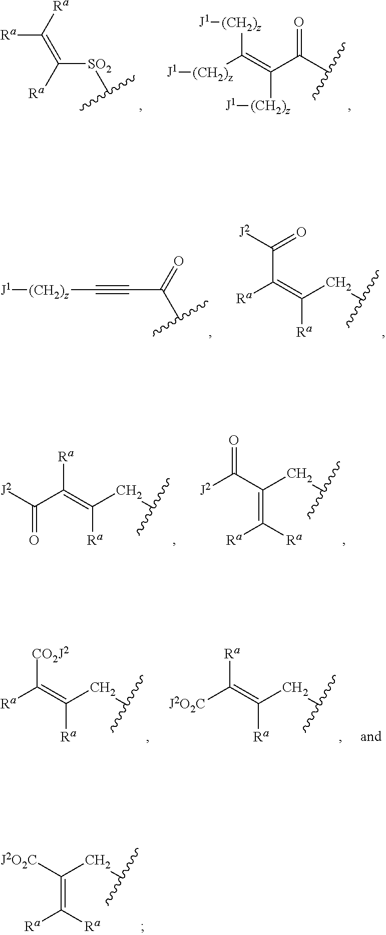

where each R^{a} is independently hydrogen, C₁₋₆alkyl, carboxy, C₁₋₆carboalkoxy, phenyl, C₂₋₇carboalkyl, R^{c}—(C(R^{b})₂)ᵣ—, R^{c}—(C(R^{b})₂)ᵣ-M-(C(R^{b})₂)ᵣ—, (R^{d})(R^{e})CH-M-(C(R^{b})₂)ᵣ—, or Het-J³-(C(R^{b})₂)ᵣ—; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; each R^{c} is independently —NR^{b}R^{b} or —OR²⁰ⁱ; R^{d} and R^{e} are each, independently, —(C(R^{b})₂)ᵣ—NR^{b}R^{b}, or —(C(R^{b})₂)ᵣ—OR^{b}; each J¹ is independently hydrogen, chlorine, fluorine, or bromine; J² is C₁₋₆alkyl or hydrogen; each M is independently —N(R^{b})—, —O—, —N[(C(R^{b})₂)_{w}—NR^{b}R^{b}]—, or —N[(C(R^{b})₂)_{w}—OR^{b}]—; each J³ is independently —N(R^{b})—, —O—, or a bond; each Het is independently a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; each r is independently 1-4; each w is independently 2-4; x is 0-1; y is 0-4, and each z is independently 1-6; wherein the sum of x+y is 2-4. In embodiments of Formula (I), (I-3), (I-4), (Ia), (Ib), (Ic), (Id), (Ii), (Ij), (Ik), (Im), (In), (Io), (Ip), (Iq), (Ir), or (I′), R⁶ is

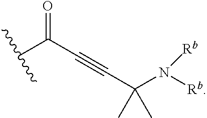

In embodiments of Formula (I), (I-3), (I-4), (Ia), (Ib), (Ic), (Id), (Ii), (Ij), (Ik), (Im), (In), (Io), (Ip), (Iq), (Ir), or (I′), R⁶ is selected from the group consisting of

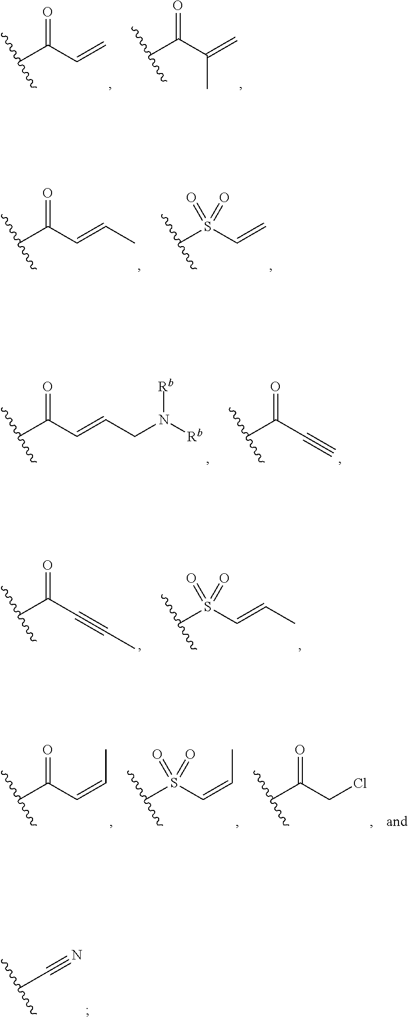

where each R^{b} is independently selected from the group consisting of hydrogen, hydroxyl, C₁-C₆ alkoxy, and C₁-C₆ alkyl, or two R^{b} optionally join to form heterocycle having 3-12 ring atoms or C₃-C₆ cycloalkyl.

In embodiments of Formula (I), (I-3), (I-4), (Ia), (Ib), (Ic), (Id), (Ii), (Ij), (Ik), (Im), (In), (Io), (Ip), (Iq), (Ir), or (I′), R⁶ is selected from the group consisting of

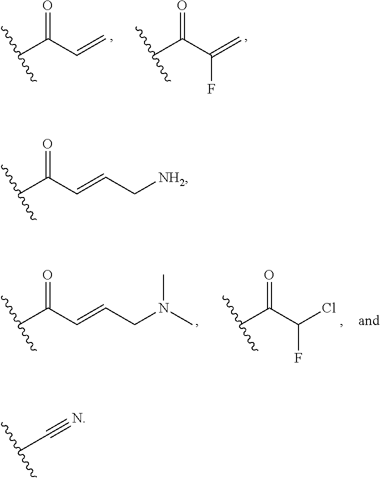

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from the group consisting of

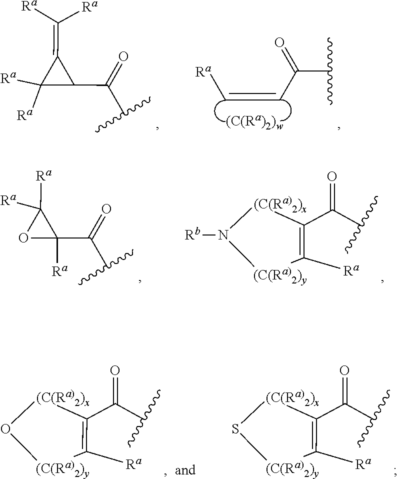

where each R^{a} is independently hydrogen, C₁₋₆ alkyl, carboxy, C₁₋₆carboalkoxy, phenyl, C₂₋₇carboalkyl, R^{c}—(C(R^{b})₂)ᵣ—, R^{c}—(C(R^{b})₂)_{w}-M-(C(R^{b})₂)ᵣ, (R^{d})(R^{e})CH-M- (C(R^{b})₂)ᵣ, or Het-J³-(C(R^{b})₂)ᵣ; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; each R^{e} is independently —NR^{b}R^{b} or —OR^{b}; R^{d} and R^{e} are each, independently, —(C(R^{b})₂)ᵣNR^{b}R^{b}, or —(C(R^{b})₂)ᵣ—OR^{b}; each J¹ is independently hydrogen, chlorine, fluorine, or bromine; J² is C₁₋₆alkyl or hydrogen; each M is independently —N(R^{b})—, —O—, —N[(C(R^{b})₂)_{w}—NR^{b}R^{b}]—, or —N[(C(R^{b})₂)_{w}—OR^{b}]—; each J³ is independently —N(R^{b})—, —O—, or a bond; each Het is independently a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; each r is independently 1-4; each w is independently 2-4; x is 0-1; y is 0-4, and each z is independently 1-6; wherein the sum of x+y is 2-4.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5 or 6 membered partially unsaturated heterocycloalkyl or a 5 or 6 membered heteroaryl optionally substituted with one, two or three R^{20k}, wherein the partially unsaturated 5 or 6 membered heterocycloalkyl or 5 or 6 membered heteroaryl comprises one, two, or three ring nitrogen atoms; and is bonded to L² through a ring nitrogen.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is not a 5 or 6 membered partially unsaturated heterocycloalkyl or a 5 or 6 membered heteroaryl optionally substituted with one, two or three R^{20k}, wherein the partially unsaturated 5 or 6 membered heterocycloalkyl or 5 or 6 membered heteroaryl comprises one, two, or three ring nitrogen atoms; and is bonded to L² through a ring nitrogen.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5 or 6 membered partially unsaturated heterocycloalkyl or a 5 or 6 membered heteroaryl optionally substituted with one, two or three R^{20k}, wherein the partially unsaturated 5 or 6 membered heterocycloalkyl or 5 or 6 membered heteroaryl comprises one, two, three, or four ring nitrogen atoms; and is bonded to L² through a ring nitrogen.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is not a 5 or 6 membered partially unsaturated heterocycloalkyl or a 5 or 6 membered heteroaryl optionally substituted with one, two or three R^{20k}, wherein the partially unsaturated 5 or 6 membered heterocycloalkyl or 5 or 6 membered heteroaryl comprises one, two, three, or four ring nitrogen atoms; and is bonded to L² through a ring nitrogen.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5 membered heteroaryl optionally substituted with one, two, or three R^{20k}, wherein the 5 membered heteroaryl comprises one, two, three, or four ring nitrogen atoms; and is bonded to L² through a ring nitrogen.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 3-5 membered heterocycloalkyl comprising at least one nitrogen ring atom optionally substituted with one, two, or three R^{20k}. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5-6 membered partially unsaturated heterocycloalkyl comprising one, two, or three ring nitrogen atoms that is optionally substituted with one, two or three R^{20k}, wherein R⁵ is (a) bonded through an R⁵ ring nitrogen to L² when L² is —C(O)—, or (b) bonded through an R⁵ ring carbon to the N(R^{4d}) of L² when L² is —C(O)N(R^{4d})—. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5-6 membered heteroaryl comprising one, two, or three ring nitrogen atoms that is optionally substituted with one, two or three R^{20k}; wherein R⁵ is (a) bonded through an R⁵ ring nitrogen to L² when L² is —C(O)—, or (b) bonded through an R⁵ ring carbon to the N(R^{4d}) of L² when L² is —C(O)N(R^{4d})—. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5-6 membered heteroaryl comprising one, two, three, or four ring nitrogen atoms that is optionally substituted with one, two or three R^{20k}; wherein R⁵ is (a) bonded through an R⁵ ring nitrogen to L² when L² is —C(O)—, or (b) bonded through an R⁵ ring carbon to the N(R^{4d}) of L² when L² is —C(O)N(R^{4d})—. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 5-10 membered spirocyclic bicyclic heterocycloalkyl comprising at least one nitrogen ring atom and optionally substituted with one, two, three, or four R^{20k}. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is a 3 membered heterocycloalkyl comprising one nitrogen ring atom optionally substituted with one, two, or three R^{20k}.

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

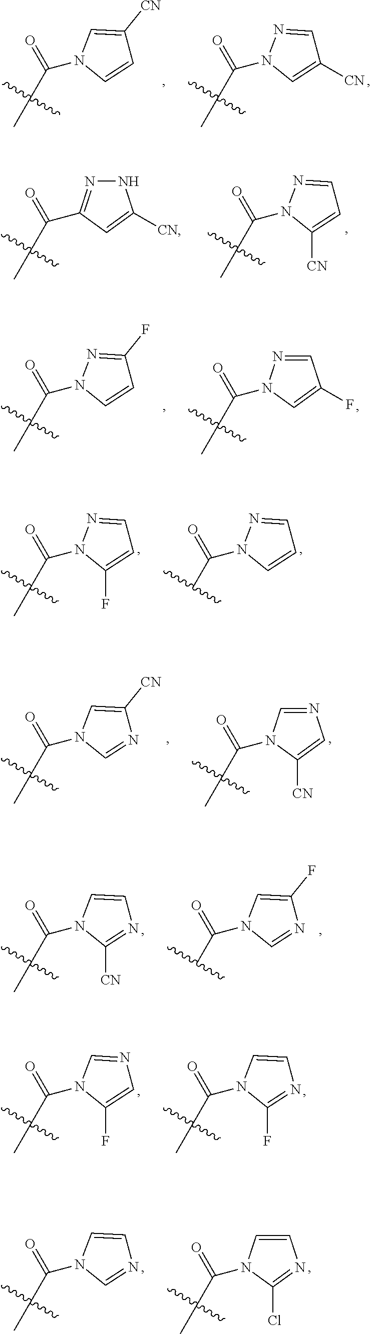

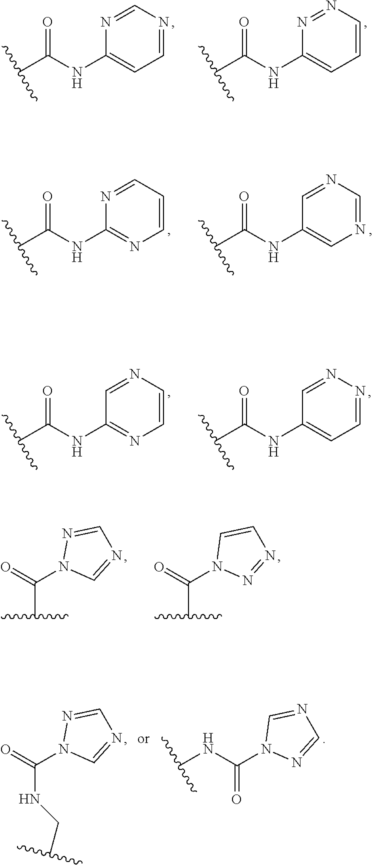

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

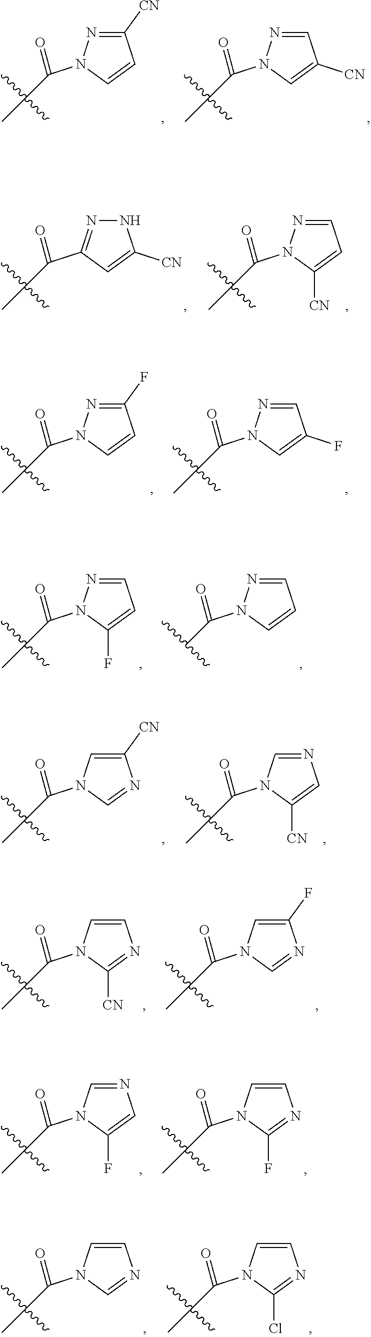

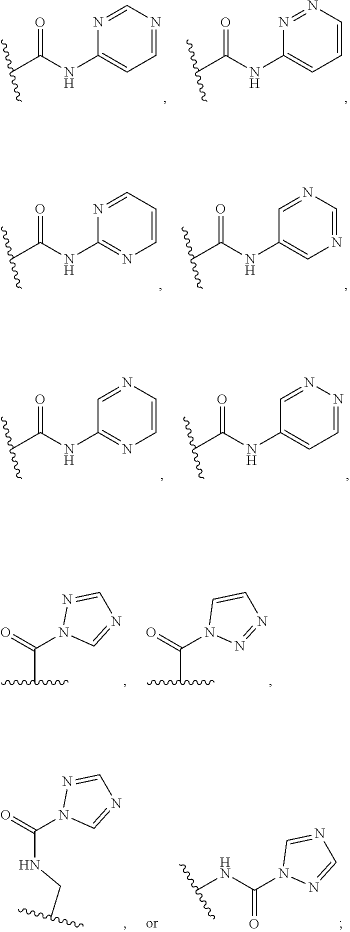

each of which is optionally substituted with one or more R^{20k}. In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

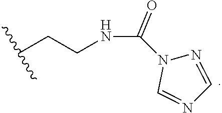 [SMILES: *CCNC(=O)n1cncn1]

In some embodiments of a compound of Formula (I), (I-3), (I-4), (II-3), (I′), (Ia), (Ib), (c), (Id), (Ii), (Ij), (Ik), (Im), (In), (Io), (Ip), (Iq), (Ir), (II), (II′), or (II″), R⁶ is

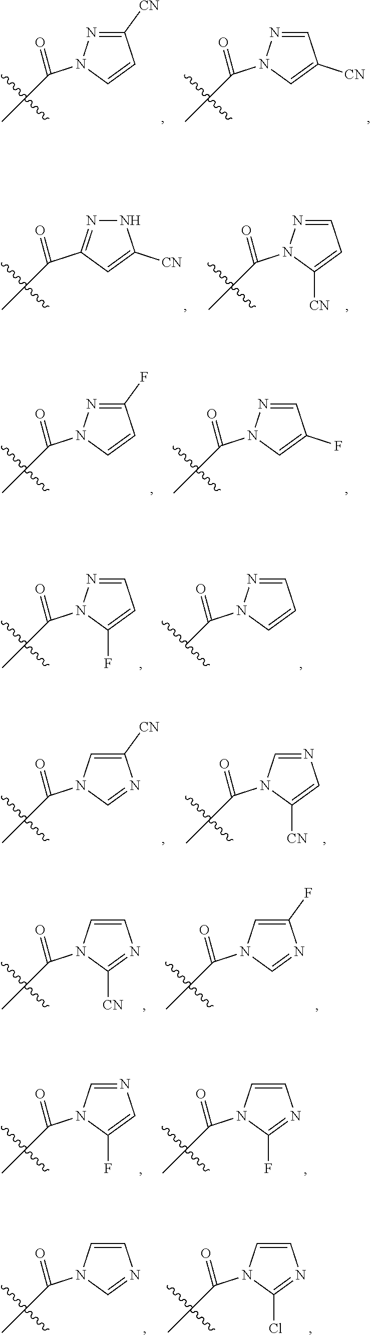

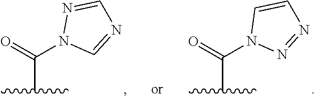 [SMILES: *C(=O)n1cncn1 ; *C(=O)n1ccnn1]

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

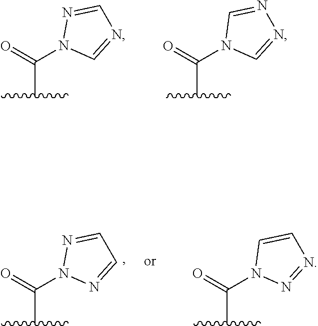 [SMILES: *C(=O)n1cncn1 ; *C(=O)n1cnnc1 ; *C(=O)n1nccn1 ; "," ; "or" ; *C(=O)n1ccnn1]

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

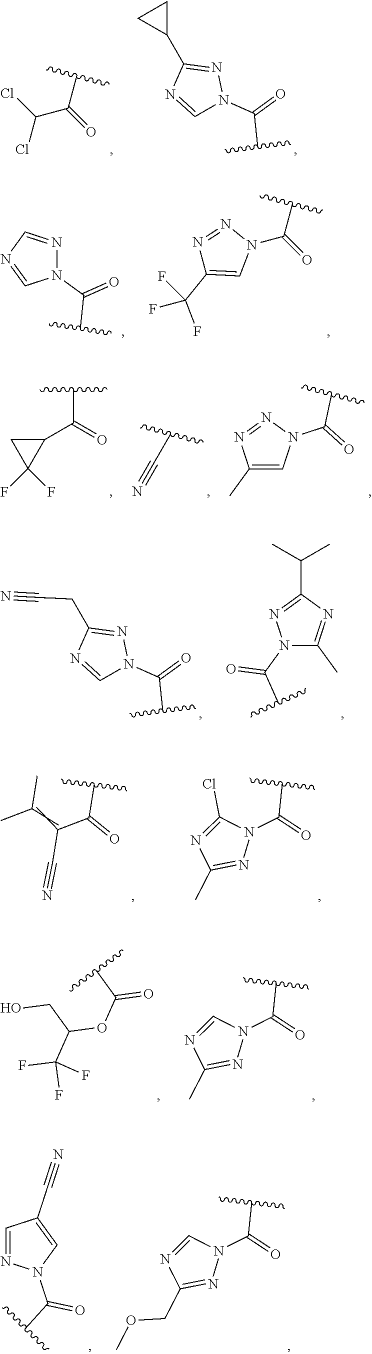

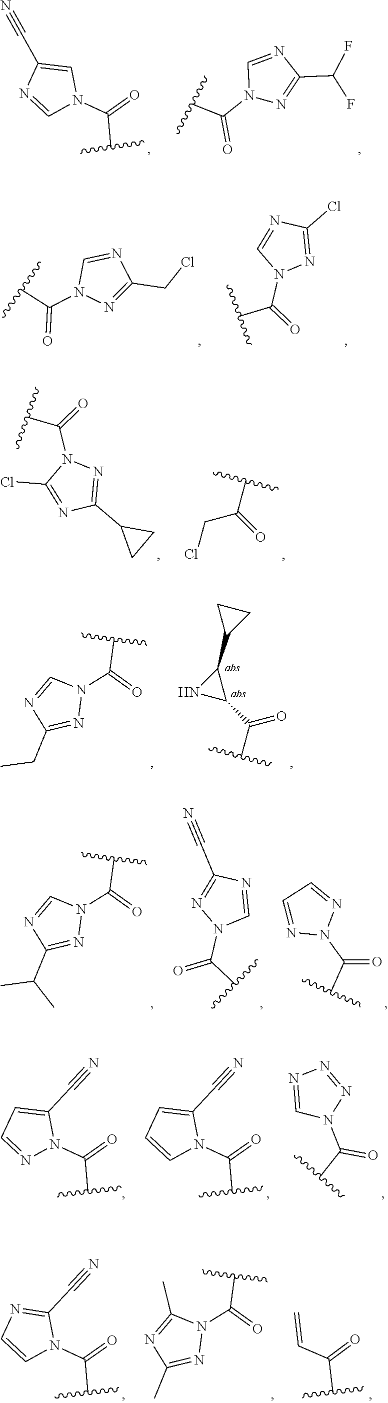

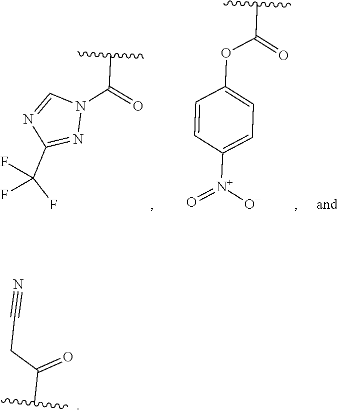 [SMILES: *C(=O)n1cnc(C(F)(F)F)n1 ; *C(=O)Oc1ccc([N+](=O)[O-])cc1 ; *C(=O)CC#N]

In some embodiments of a compound of Formula (I), (I-3), (I-4), (Ia), (Ib), (Ic), (Id), (Ii), (Ij), (Ik), (Im), (In), (Io), (Ip), (Iq), (Ir), or (I′), or a pharmaceutically acceptable salt or solvate thereof,
L² is a bond, —C(O)NH—, —NHC(O)—, or —C(O)—; and
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{2k}.

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), L² is —C(O)—; and R⁵ is a C₃₋₁₂cycloalkyl optionally substituted with one, two or three R^{20k}.

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), L² is —C(O)—; and R⁵ is a cyclopropyl optionally substituted with one, two or three R^{20k} independently selected from halogen and CN.

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

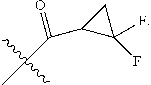 [SMILES: *C(=O)C1CC1(F)F]

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

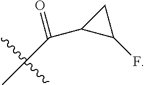 [SMILES: *C(=O)C1CC1F]

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

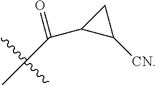 [SMILES: *C(=O)C1CC1C#N]

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

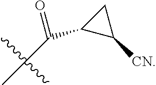 [SMILES: *C(=O)[C@@H]1C[C@H]1C#N]

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

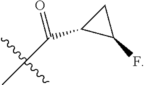 [SMILES: *C(=O)[C@@H]1C[C@H]1F]

In some embodiments of a compound of Formula (I), (I-3), (I-4), (Ia), (Ib), (Ic), (Id), (Ii), (Ij), (Ik), (Im), (In), (Io), (Ip), (Iq), (Ir), or (I′), L² is a bond, —C(O)NH—, —NHC(O)—, or —C(O)—; L² is bonded to a carbon atom of R⁵; and R⁵ is selected from —CN, C₁₋₆alkyl, C₂₋₆alkenyl, C₂₋₆alkynyl, C₃₋₁₂cycloalkyl, C₆₋₁₂aryl, and C₁₋₁₁heteroaryl, wherein C₁₋₆alkyl, C₂₋₆alkenyl, C₂₋₆alkynyl, C₃₋₁₂cycloalkyl, C₆₋₁₂aryl, and C₁₋₁₁heteroaryl, are optionally substituted with one, two, or three R^{20k}. In some embodiments of a compound of Formula (I), (I-3), (I-4), (Ia), (Ib), (Ic), (Id), (Ii), (Ij), (Ik), (Im), (In), (Io), (Ip), (Iq), (Ir), or (I′), L² is —C(O)—; L² is bonded to a carbon atom of R⁵; and R⁵ is selected from C₁₋₆alkyl, C₂₋₆alkenyl, C₂₋₆alkynyl, and C₁₋₁₁heteroaryl, wherein C₁₋₆alkyl, C₂₋₆alkenyl, C₂₋₆alkynyl, and C₁₋₁₁ heteroaryl, are optionally substituted with one, two, or three R^{20k}. In some embodiments of a compound of Formula (I), (I-3), (I-4), (Ia), (Ib), (Ic), (Id), (Ii), (Ij), (Ik), (Im), (In), (Io), (Ip), (Iq), (Ir), or (I′), L² is —C(O)—; L² is bonded to a carbon atom of R⁵; and R⁵ is selected from C₁₋₆alkyl, C₂₋₆alkenyl, C₂₋₆alkynyl, C₃₋₁₀cycloalkyl, and 5-6 membered heteroaryl, wherein C₁₋₆alkyl, C₂₋₆alkenyl, C₂₋₆alkynyl, C₃₋₁₀cycloalkyl, and 5-6 membered heteroaryl are optionally substituted with one, two, or three R^{20k}. In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), L² is —C(O)—; R⁵ is a heteroaryl having the formula:

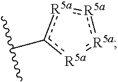

R^{5a} is independently O, S, CH, C(R^{20k}), N, NH, or N(R^{20k}); R⁵ comprises 0-3 independent R^{20k}; and 0-4 R^{5a} are independently N, NH, or N(R^{20k}). In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), L² is —C(O)—; and R⁵ is a heteroaryl having the formula:

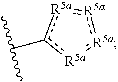

R^{5a} is independently CH, C(R^{20k}), N, NH, or N(R^{20k}); R⁵ comprises 0-3 independent R^{20k}; and 0-4 R^{5a} are independently N, NH, or N(R²⁰ⁱ). In some embodiments of a compound of Formula (I), (I-3), (I-4), (II-3), (I′), (Ia), (Ib), (Ic), (Id), (Ii), (Ij) (Ik), (Im), (In), (Io), (Ip), (Iq), (Ir), (II), (II′), or (II″), L² is —C(O)—; R⁵ is

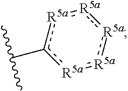

R^{5a} is independently CH, C(R^{20k}), CH(R^{20k}), CH₂, C(R^{20k})₂, N, NH, or N(R²⁰ⁱ); R⁵ comprises 0-3 independent R^{20k}; and 0-4 R^{5a} are independently N, NH, or N(R^{20k}). R^{5a} is independently CH, C(R^{20k}), CH(R^{20k}), CH₂, C(R²⁰ⁱ)₂, N, NH, or N(R^{20k}); R⁵ comprises 0-3 independent R^{20k}; and 0-4 R^{5a} are N, NH, or N(R^{20k}). In some embodiments of a compound of Formula (I), (I-3), (I-4), (II-3), (Ia), (Ib), (Ic), (Id), (Ii), (Ij), (Ik), (Im), (In), (Io), (Ip), (Iq), (Ir), or (I′), L² is —C(O)—; and R⁵ is selected from C₂₋₆alkenyl and C₂₋₆alkynyl, wherein C₂₋₆alkenyl and C₂₋₆alkynyl are optionally substituted with one, two, or three R^{20k}.

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is

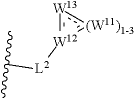

wherein each W¹¹ and W¹³ are independently selected from O, S, CH, CR^{20k}, CH₂, CHR^{20k}, C(R²⁰ⁱ)₂, N, NH, and NR^{20k} and W¹² is independently selected from C, CH, CR^{20k}, and N, wherein at least one of W¹¹ and W¹³ is N, NH, or NR^{20k} or W¹² is N, and wherein the ring including W¹¹, W¹², and W¹³ is not an aromatic ring. [symbol] indicates a single or double bond such that all valences are satisfied. It will be understood that when W¹¹ is N, NH, or NR^{20k}, then only one, two or three W¹¹ may be N, NH, or NR^{20k} and each W¹¹ may optionally be different and may each be independently selected from O, S, CH, CR^{20k}, CH₂, CHR^{20k}, C(R²⁰ⁱ)₂, N, NH, and NR^{20k} with the requirement that at least one of W¹¹ and W¹³ is N, NH, or NR^{20k} or W¹² is N. For example, when R⁶ includes three W¹¹, one W¹¹ may be N, a second W¹¹ may be CR^{20k}, and a third W¹¹ may be CH₂.

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is selected from

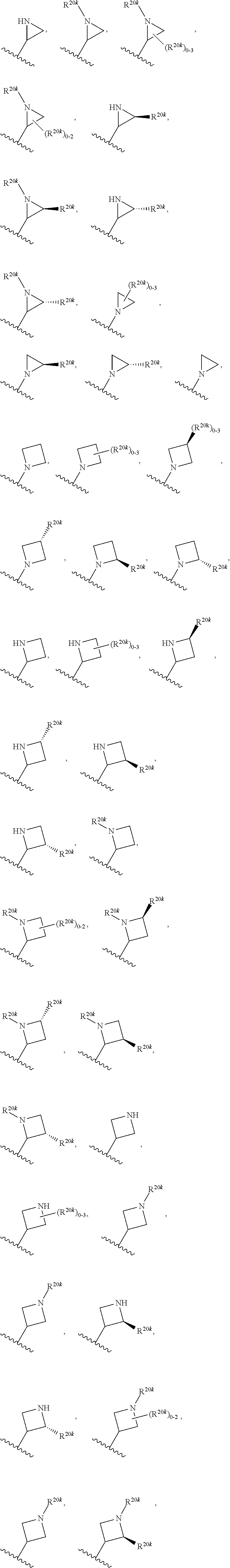

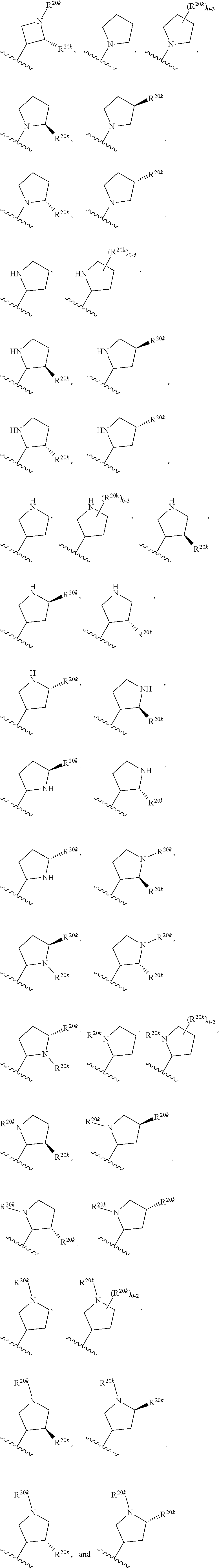

In some embodiments of a compound of Formula (I), (I-3), (1-4), (11-3), (I′), (Ia), (Ib), (Ic), (Id), (Ii), (Ij), (Ik), (Im), (In), (Io), (Ip), (Iq), (Ir), (II), (II′, or (II″), R⁵ is selected from

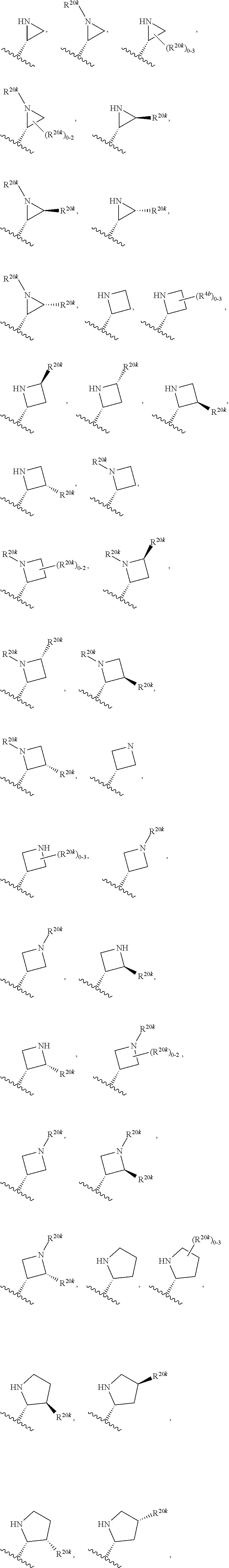

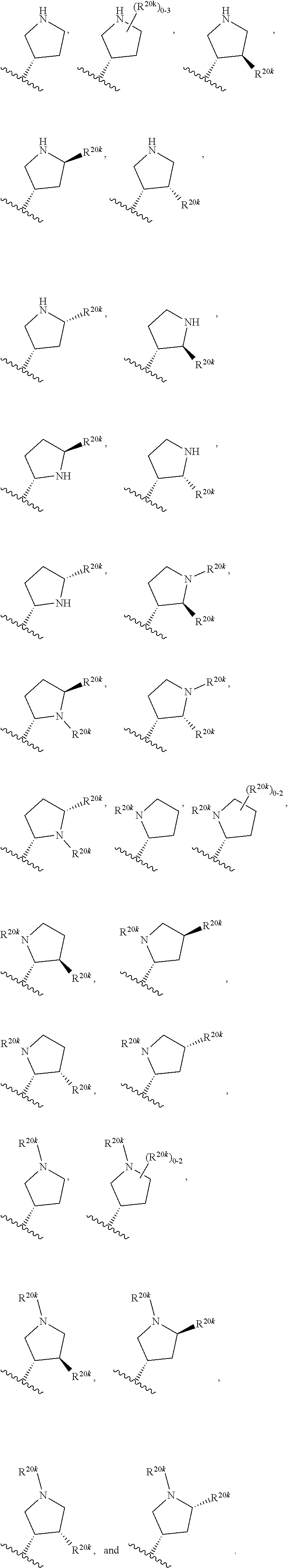

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is selected from

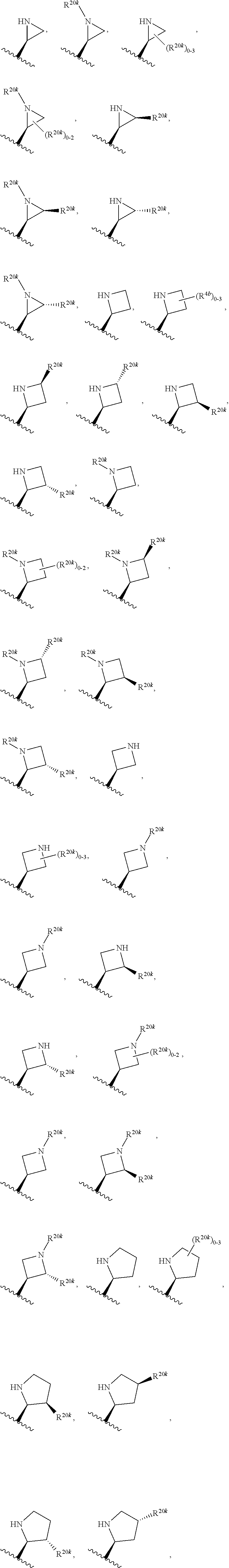

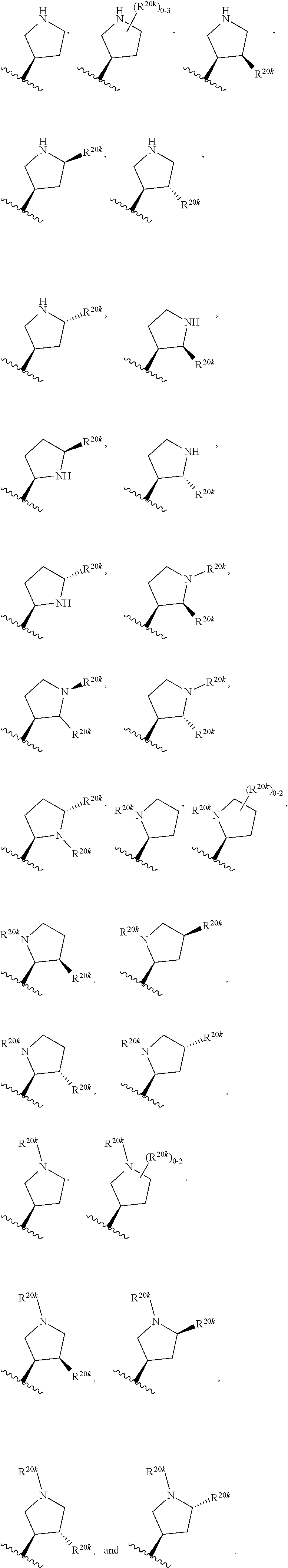

In some embodiments of a compound of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is selected from,

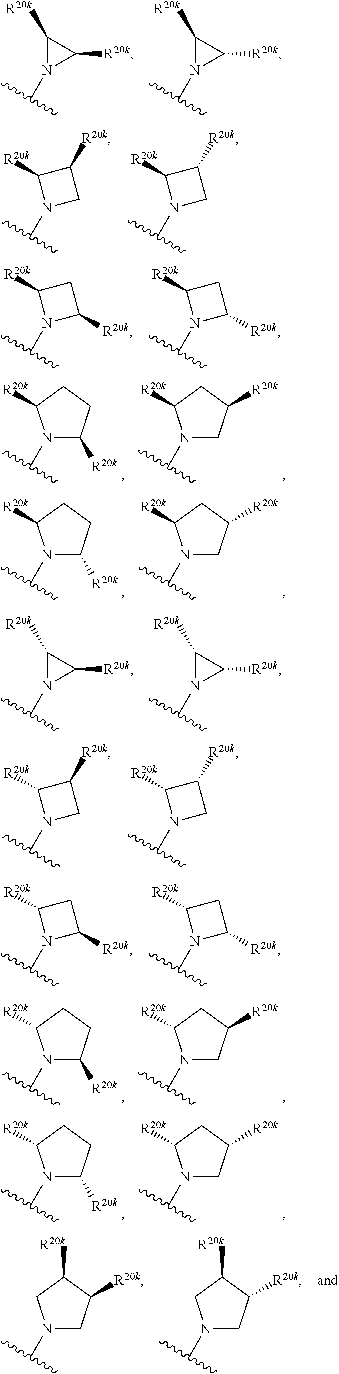

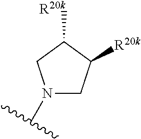

wherein each R^{20k} is independently selected and are optionally different.

The individual embodiments herein below, or combinations thereof, (e.g., embodiments of R⁵ or R⁶) are applicable to compounds of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), or a pharmaceutically acceptable salt or solvate thereof. In embodiments of the formulae above, R⁵ is selected from

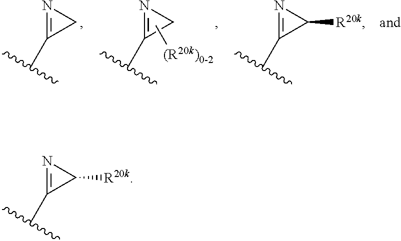

In embodiments of the formulae above, R⁵ is

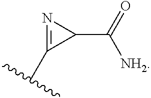 [SMILES: *C1=NC1C(N)=O]

In embodiments of the formulae above, R⁵ is

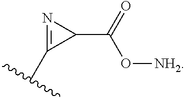 [SMILES: *C1=NC1C(=O)ON]

In embodiments of the formulae above, R⁶ is independently selected from

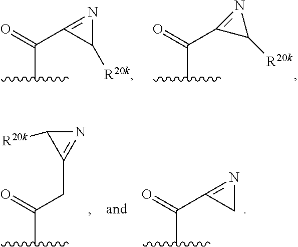

In embodiments of the formulae above, R⁶ is

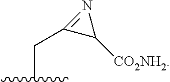

In embodiments of the formulae above, R⁶ is

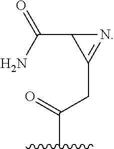 [SMILES: *C(=O)CC1=NC1C(N)=O]

In embodiments of the formulae above, R⁵ is

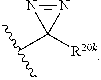

In embodiments of the formulae above, R⁶ is selected from

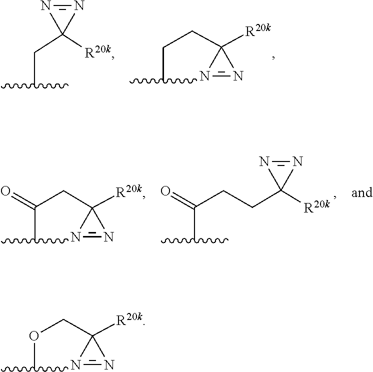

In embodiments of the formulae above, R⁶ is selected from

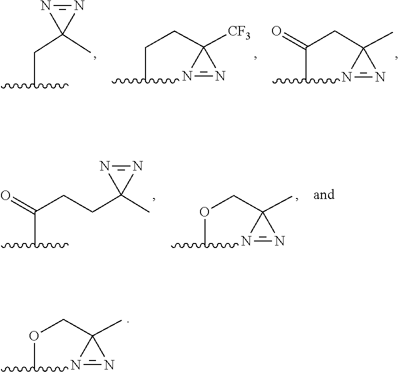

In embodiments of the formulae above, R⁶ is selected from:

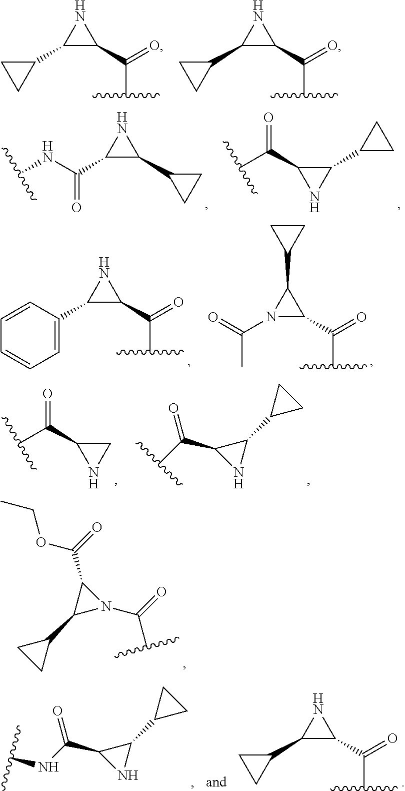

In embodiments of the formulae above, R⁶ is selected from:

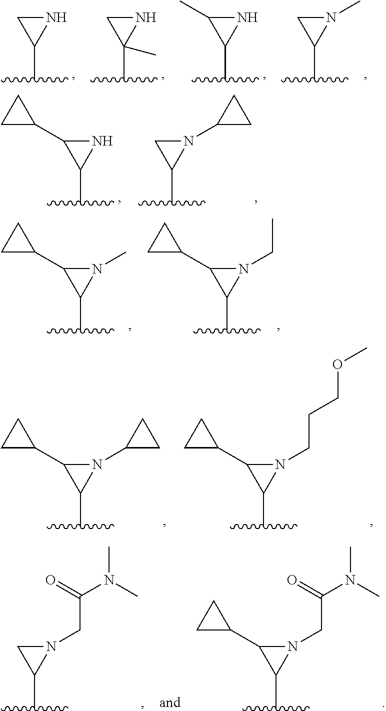

In embodiments of the formulae above, R⁶ is selected from:

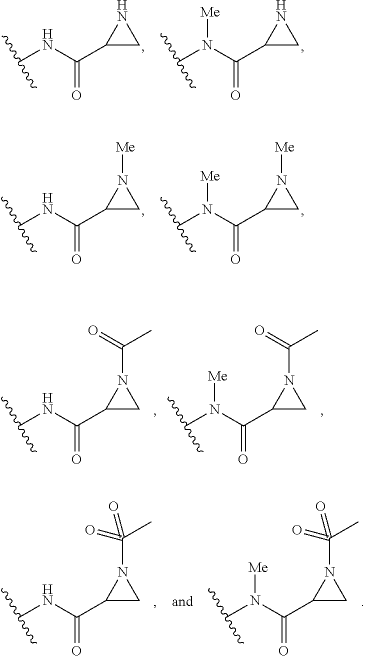

In embodiments of the formulae above, R⁶ is selected from:

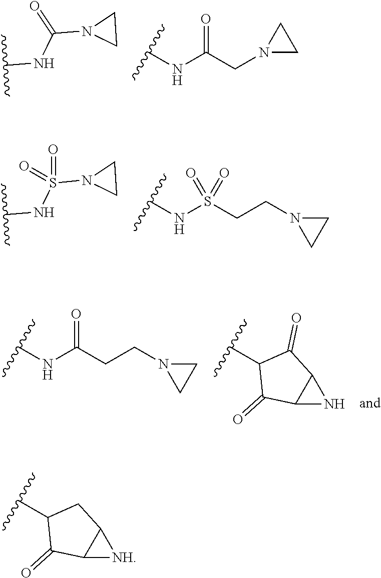

In embodiments of the formulae above, R⁶ is selected from:

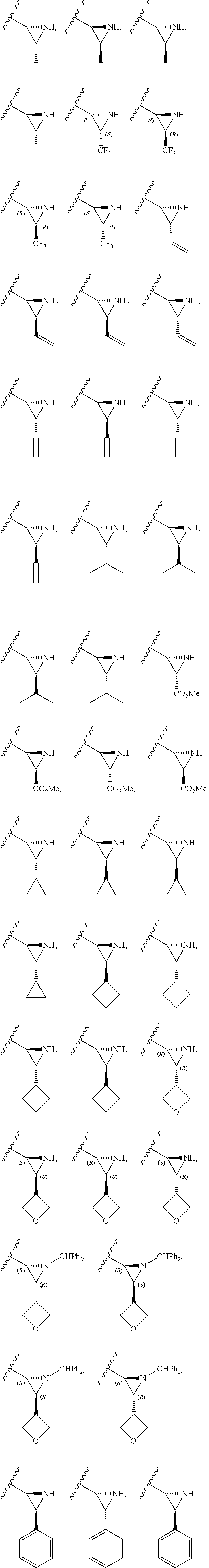

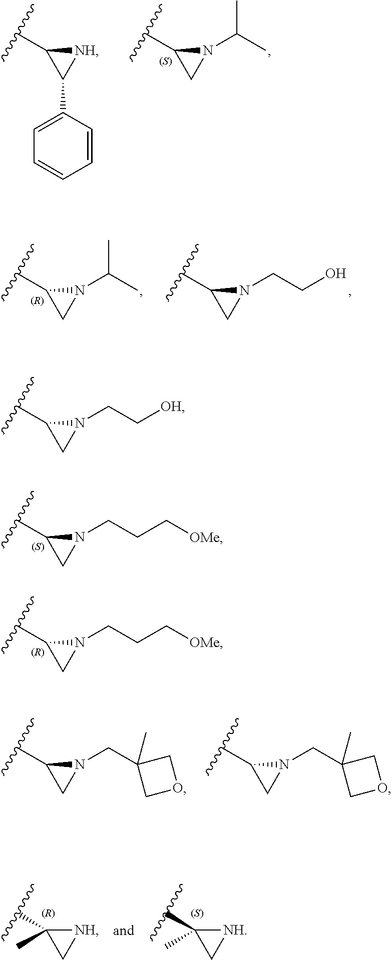

In embodiments of the formulae above, R⁵ is independently a 5 membered spirocyclic bicyclic heterocycloalkyl comprising at least one nitrogen ring atom and optionally substituted with one, two, three, or four R^{20k}. In embodiments of the formulae above, R⁵ is independently a 6 membered spirocyclic bicyclic heterocycloalkyl comprising at least one nitrogen ring atom and optionally substituted with one, two, three, or four R^{20k}. In embodiments of the formulae above, R⁵ is independently a 7 membered spirocyclic bicyclic heterocycloalkyl comprising at least one nitrogen ring atom and optionally substituted with one, two, three, or four R^{20k}. In embodiments of the formulae above, R⁵ is independently a 8 membered spirocyclic bicyclic heterocycloalkyl comprising at least one nitrogen ring atom and optionally substituted with one, two, three, or four R^{20k}. In embodiments of the formulae above, R⁵ is independently a 9 membered spirocyclic bicyclic heterocycloalkyl comprising at least one nitrogen ring atom and optionally substituted with one, two, three, or four R^{2k}. In embodiments of the formulae above, R⁵ is independently a 10 membered spirocyclic bicyclic heterocycloalkyl comprising at least one nitrogen ring atom and optionally substituted with one, two, three, or four R^{20k}

In embodiments of the formulae above, R⁵ is selected from

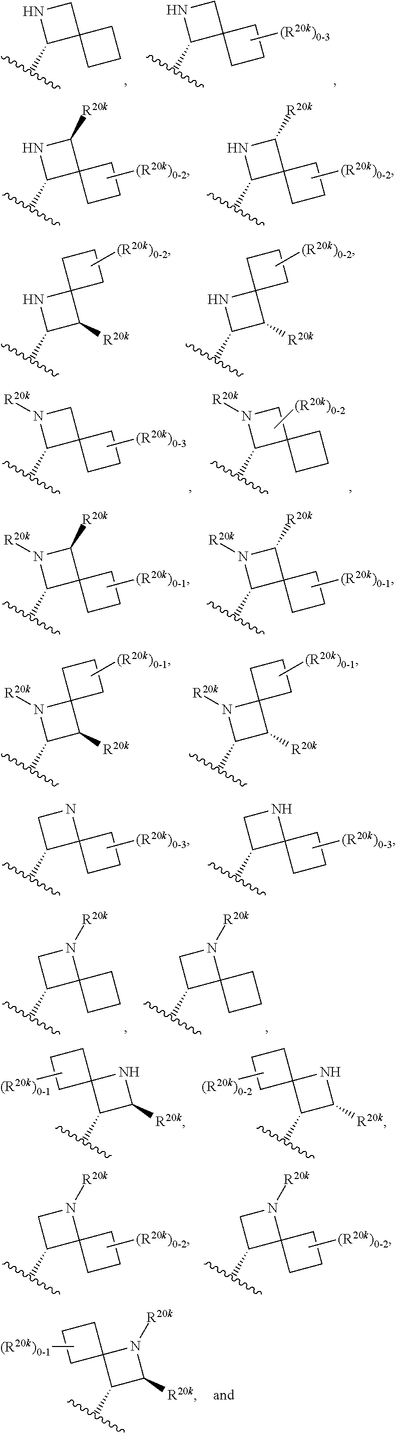

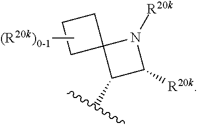

In embodiments of the formulae above, R⁵ is selected from

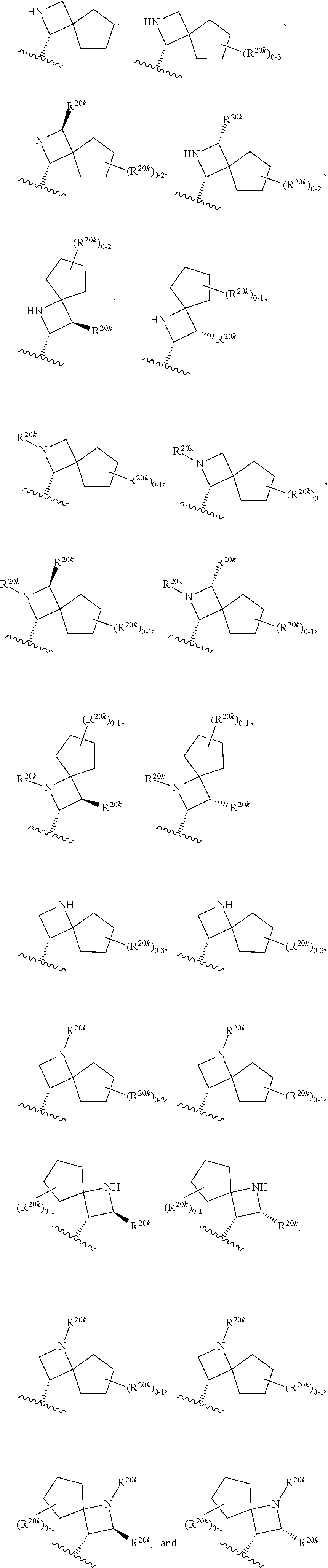

In embodiments of the formulae above, R⁵ is selected from

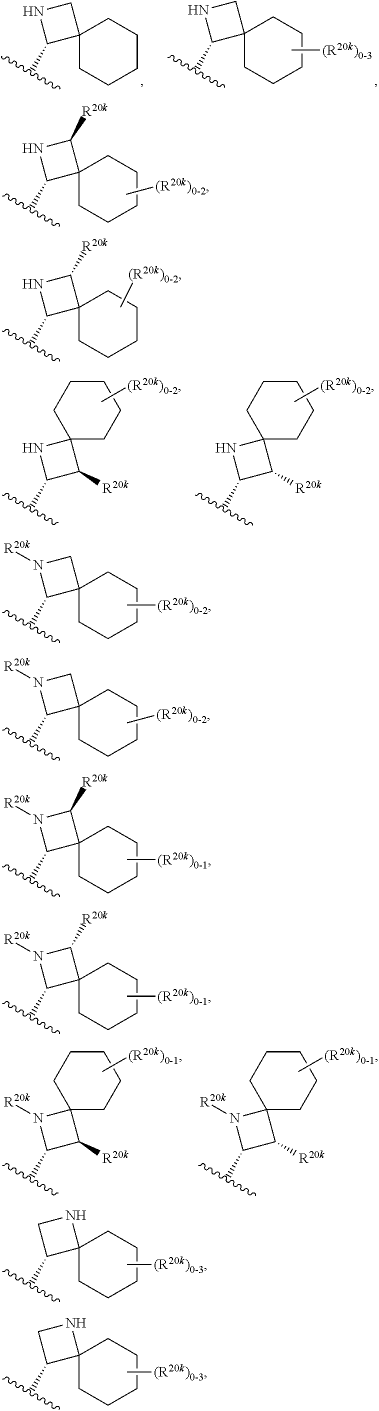

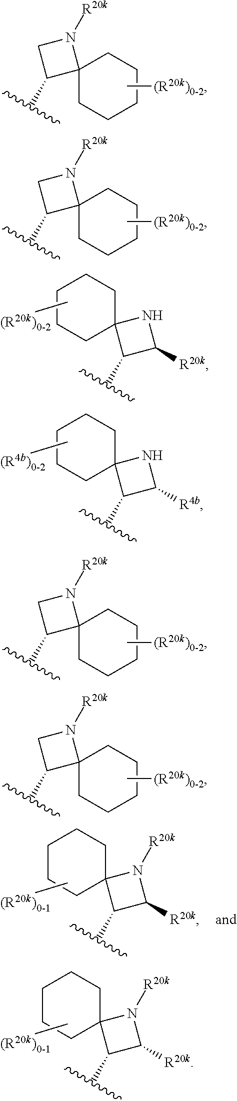

In embodiments of the formulae above, R⁵ is selected from

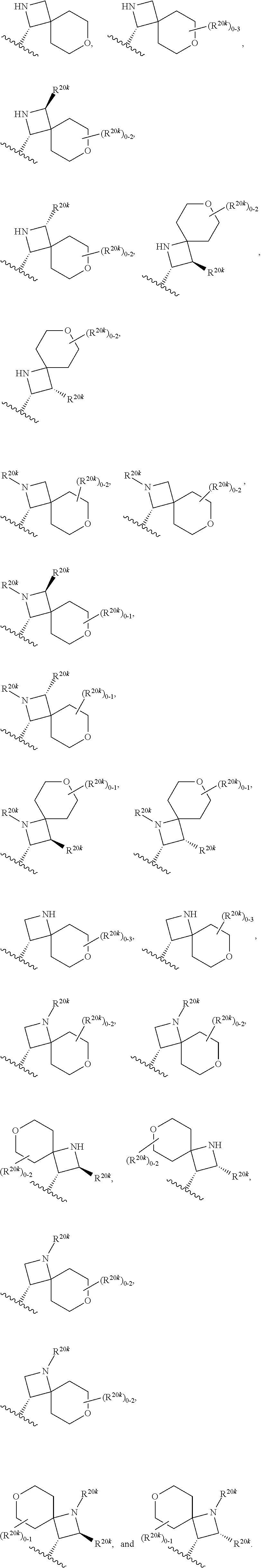

In embodiments of the formulae above, R⁵ is selected from

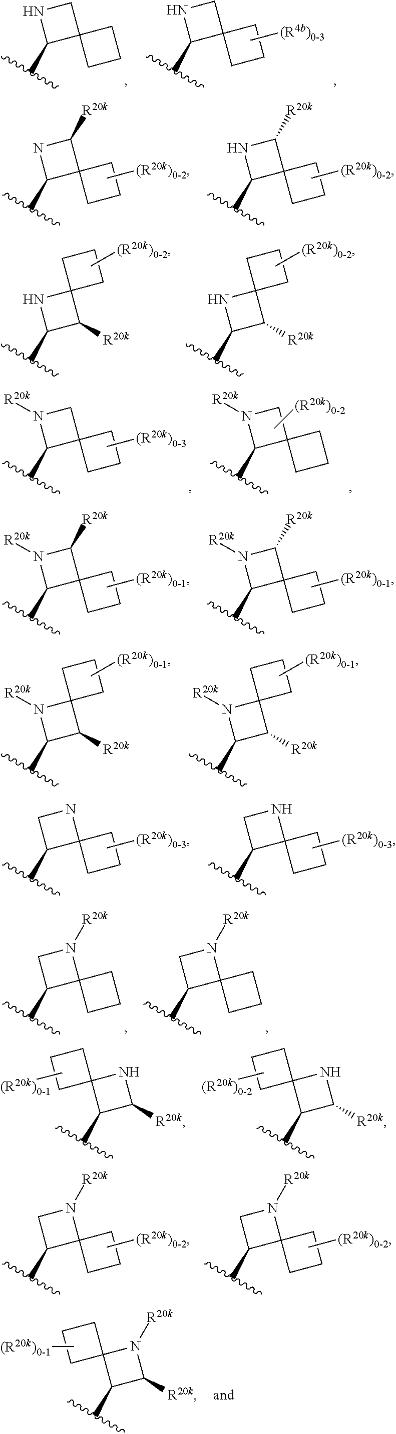

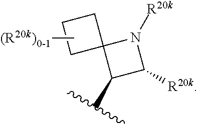

In embodiments of the formulae above, R⁵ is selected from

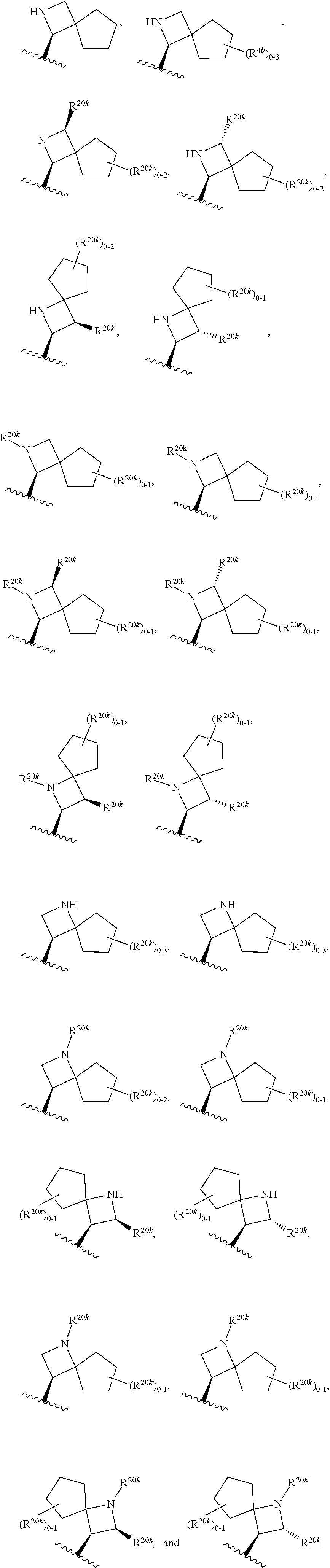

In embodiments of the formulae above, R⁵ is selected from

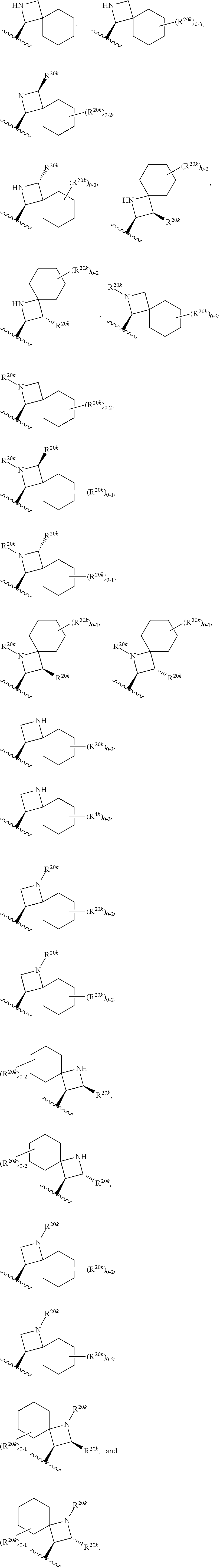

In embodiments of the formulae above, R⁵ is selected from

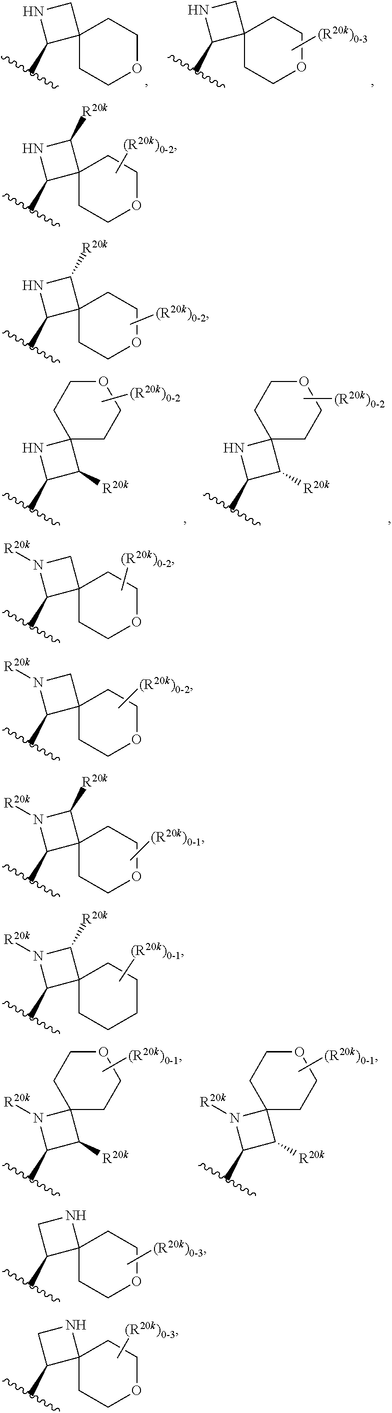

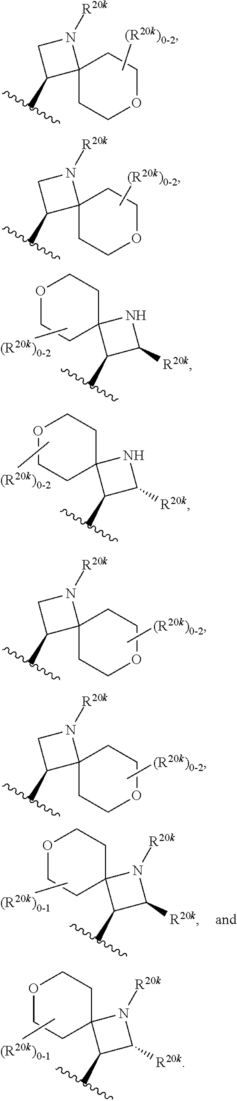

In embodiments of the formulae above, R⁵ is selected from

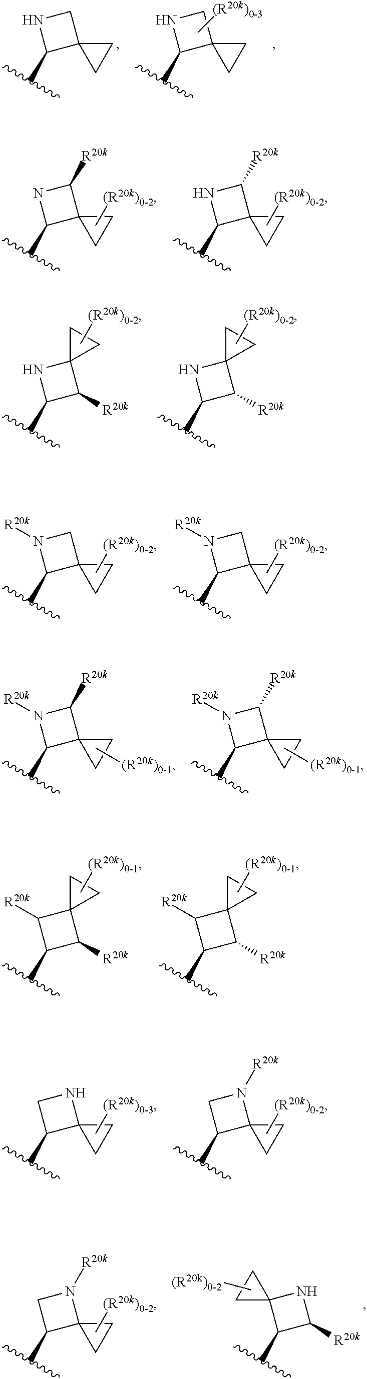

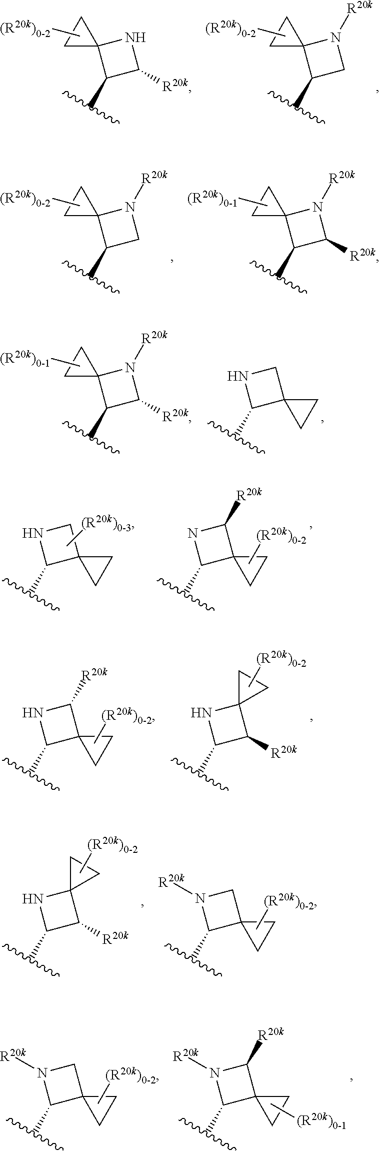

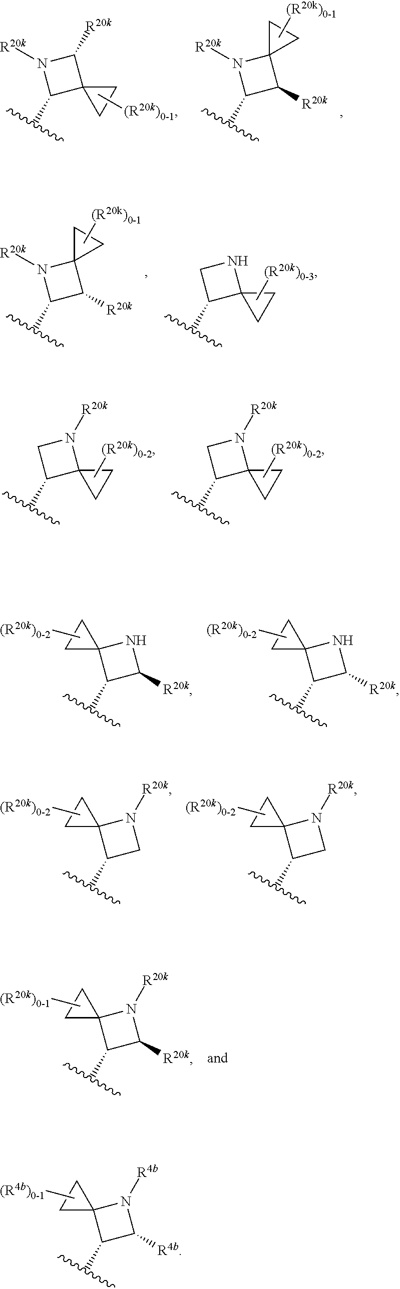

In some embodiments of a compound of Formula (II), (II′), (II″), (IV-3), (IV-4), (IV), (IVa), or (IV′), or a pharmaceutically acceptable salt or solvate thereof, Y is C(O).

The individual embodiments herein below, or combinations thereof, (e.g., embodiments of Y, X, U, W, Z, V, or J) are applicable to compounds of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV′), or a pharmaceutically acceptable salt or solvate thereof. In embodiments of the formulae above, Y is N. In embodiments of the formulae above, Y is C(R²). In embodiments of the formulae above, Y is C(R²)(R^{2c}). In embodiments of the formulae above, Y is S(O). In embodiments of the formulae above, Y is S(O)₂. In embodiments of the formulae above, X is N. In embodiments of the formulae above, X is C(R³). In embodiments of the formulae above, X is C(R³)(R³). In embodiments of the formulae above, X is N(R³). In embodiments of the formulae above, U is N. In embodiments of the formulae above, U is C(R^{2c}). In embodiments of the formulae above, U is C(R^{2c})(R^{2c}). In embodiments of the formulae above, U is N(R^{2b}). In embodiments of the formulae above, U is S(O). In embodiments of the formulae above, U is S(O)₂. In embodiments of the formulae above, U is C(O). In embodiments of the formulae above, W is a N. In embodiments of the formulae above, W is a C(R¹⁴). In embodiments of the formulae above, W is a N(R^{18b}). In embodiments of the formulae above, W is a C(R¹⁸)(R^{18a}). In embodiments of the formulae above, W is a C(O). In embodiments of the formulae above, W is a S(O). In embodiments of the formulae above, W is a S(O)₂. In embodiments of the formulae above, Z is N. In embodiments of the formulae above, Z is C(R⁸). In embodiments of the formulae above, Z is N(R^{8b}). In embodiments of the formulae above, Z is C(R⁸)(R^{8a}). In embodiments of the formulae above, Z is C(O). In embodiments of the formulae above, Z is S(O). In embodiments of the formulae above, Z is S(O)₂. In embodiments of the formulae above, V is N(R^{16b}). In embodiments of the formulae above, V is N. In embodiments of the formulae above, V is C(R¹⁶)(R^{16a}) In embodiments of the formulae above, V is C(R¹⁶). In embodiments of the formulae above, V is N(R^{17b}). In embodiments of the formulae above, V is C(R¹⁷)(R^{16a}). In embodiments of the formulae above, V is C(R¹⁷). In embodiments of the formulae above, J is N(R^{16b}). In embodiments of the formulae above, J is N. In embodiments of the formulae above, J is C(R¹⁶)(R^{16a}). In embodiments of the formulae above, J is C(R¹⁶). In embodiments of the formulae above, J is N(R¹⁷). In embodiments of the formulae above, J is C(R¹⁷)(R^{16a}). In embodiments of the formulae above, J is C(R¹⁷).

The individual embodiments herein below, or combinations thereof, (e.g., embodiments of L⁷, W¹, W², W³, W⁴, or W⁵) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof.

In embodiments of the formulae above, L⁷ is a bond.

In embodiments of the formulae above, W¹ is N(R¹). In embodiments of the formulae above, W¹ is N(R⁴). In embodiments of the formulae above, W¹ is C(R¹)(R¹). In embodiments of the formulae above, W¹ is C(R¹)(R⁴). In embodiments of the formulae above, W¹ is C(R⁴)(R⁴). In embodiments of the formulae above, W¹ is C(O). In embodiments of the formulae above, W¹ is S. In embodiments of the formulae above, W¹ is O. In embodiments of the formulae above, W¹ is S(O). In embodiments of the formulae above, W¹ is S(O)₂. In embodiments of the formulae above, W¹ is NH. In embodiments of the formulae above, W¹ is CH₂.

In embodiments of the formulae above, W³ is N(R¹). In embodiments of the formulae above, W³ is N(R⁴). In embodiments of the formulae above, W³ is C(R¹)(R¹). In embodiments of the formulae above, W³ is C(R¹)(R⁴). In embodiments of the formulae above, W³ is C(R⁴)(R⁴). In embodiments of the formulae above, W³ is C(O). In embodiments of the formulae above, W³ is S. In embodiments of the formulae above, W³ is O. In embodiments of the formulae above, W³ is S(O). In embodiments of the formulae above, W³ is S(O)₂. In embodiments of the formulae above, W³ is NH. In embodiments of the formulae above, W³ is CH₂.

In embodiments of the formulae above, W¹ and W³ are independently CH₂.

In embodiments of the formulae above, W² is a bond. In embodiments of the formulae above, W² is N(R¹). In embodiments of the formulae above, W² is N(R⁴). In embodiments of the formulae above, W² is C(R¹)(R¹). In embodiments of the formulae above, W² is C(R¹)(R⁴). In embodiments of the formulae above, W³ is C(R⁴)(R⁴). In embodiments of the formulae above, W² is C(O). In embodiments of the formulae above, W² is S. In embodiments of the formulae above, W² is O. In embodiments of the formulae above, W² is S(O). In embodiments of the formulae above, W² is S(O)₂. In embodiments of the formulae above, W² is NH. In embodiments of the formulae above, W² is CH₂.

In embodiments of the formulae above, W⁴ is C(R¹)(R¹). In embodiments of the formulae above, W⁴ is C(R¹)(R⁴). In embodiments of the formulae above, W⁴ is C(R⁴)(R⁴). In embodiments of the formulae above, W⁴ is CH₂.

In embodiments of the formulae above, W⁴ is selected from C(R¹)(R¹), C(R¹)(R⁴), and C(R⁴)(R⁴).

In embodiments of the formulae above, W⁵ is N. In embodiments of the formulae above, W⁵ is C(R¹). In embodiments of the formulae above, W⁵ is C(R⁴). In embodiments of the formulae above, W⁵ is CH.

The individual embodiments herein below, or combinations thereof, (e.g., embodiments of L⁷, W¹, W², W³, W⁴, or W⁵) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), or (IV), or a pharmaceutically acceptable salt or solvate thereof. In embodiments of the formulae above, W⁴ is C(O). In embodiments of the formulae above, W⁴ is S(O). In embodiments of the formulae above, W⁴ is S(O)₂. In embodiments of the formulae above, W⁴ is selected from C(R¹)(R¹), C(R¹)(R⁴), C(R⁴)(R⁴), S(O), and S(O)₂.

The individual embodiments herein below, or combinations thereof, (e.g., embodiments of L⁷) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, L⁷ is a bond. In embodiments of the formulae above, L⁷ is —O—. In embodiments of the formulae above, L⁷ is —N(R¹⁴)—. In embodiments of the formulae above, L⁷ is —C(O)—. In embodiments of the formulae above, L⁷ is —S—. In embodiments of the formulae above, L⁷ is —S(O)₂—. In embodiments of the formulae above, L⁷ is —S(O)—. In embodiments of the formulae above, L⁷ is —NH—. In embodiments of the formulae above, L⁷ is CH₂. In embodiments of the formulae above, L⁷ is —OCH₂—. In embodiments of the formulae above, L⁷ is —N(H)CH₂—. In embodiments of the formulae above, L⁷ is —C(O)CH₂—. In embodiments of the formulae above, L⁷ is —SCH₂—. In embodiments of the formulae above, L⁷ is —S(O)₂CH₂—. In embodiments of the formulae above, L⁷ is —S(O)CH₂—. In embodiments of the formulae above, L⁷ is —P(O)(CH₃)CH₂—. In embodiments of the formulae above, L⁷ is —CH₂CH₂—. In embodiments of the formulae above, L⁷ is —CH₂O—. In embodiments of the formulae above, L⁷ is —CH₂N(H)—. In embodiments of the formulae above, L⁷ is —CH₂C(O)—. In embodiments of the formulae above, L⁷ is —CH₂S—. In embodiments of the formulae above, L⁷ is —CH₂S(O)₂—. In embodiments of the formulae above, L⁷ is —CH₂S(O)—. In embodiments of the formulae above, L⁷ is —CH₂P(O)CH₃—. In embodiments of the formulae above, L⁷ is —N(H)C(O)—. In embodiments of the formulae above, L⁷ is —N(H)P(O)CH₃—. In embodiments of the formulae above, L⁷ is —C(O)N(H)—. In embodiments of the formulae above, L⁷ is —CH₂CH₂CH₂—. In embodiments of the formulae above, L⁷ is —OCH₂CH₂—. In embodiments of the formulae above, L⁷ is —N(H)CH₂CH₂—. In embodiments of the formulae above, L⁷ is —C(O)CH₂CH₂—. In embodiments of the formulae above, L⁷ is —SCH2CH₂—. In embodiments of the formulae above, L⁷ is —S(O)₂CH₂CH₂—. In embodiments of the formulae above, L⁷ is —S(O)CH₂CH₂—. In embodiments of the formulae above, L⁷ is —P(O)(CH₃)CH₂CH₂—. In embodiments of the formulae above, L⁷ is —CH₂CH₂O—. In embodiments of the formulae above, L⁷ is —CH₂CH₂N(H)—. In embodiments of the formulae above, L⁷ is —CH₂CH₂C(O)—. In embodiments of the formulae above, L⁷ is —CH₂CH₂S—. In embodiments of the formulae above, L⁷ is —CH₂CH₂S(O)₂—. In embodiments of the formulae above, L⁷ is —CH₂CH₂S(O)—. In embodiments of the formulae above, L⁷ is —CH₂CH₂P(O)(CH₃)—. In embodiments of the formulae above, L⁷ is —CH₂CH₂CH₂CH₂—. In embodiments of the formulae above, L⁷ is C₁₋₄alkyl optionally substituted with one, two or three R^{20a}. In embodiments of the formulae above, L⁷ is C₁₋₄alkyl optionally substituted with one, two or three R^{20a}. In embodiments of the formulae above, L⁷ is C₂alkyl optionally substituted with one, two or three R^{20a}. In embodiments of the formulae above, L⁷ is C₃alkyl optionally substituted with one, two or three R^{20a}. In embodiments of the formulae above, L⁷ is C₄alkyl optionally substituted with one, two or three R^{20a}. In embodiments of the formulae above, L⁷ is 2-4 membered heteroalkyl linker optionally substituted with one, two or three R^{20a}. In embodiments of the formulae above, L⁷ is 2 membered heteroalkyl linker optionally substituted with one, two or three R^{20a}. In embodiments of the formulae above, L⁷ is 3 membered heteroalkyl linker optionally substituted with one, two or three R^{20a}. In embodiments of the formulae above, L⁷ is 4 membered heteroalkyl linker optionally substituted with one, two or three R^{20a}. In embodiments of the formulae above, L⁷ is —CH₂C(O)N(H)—. In embodiments of the formulae above, L⁷ is —CH₂CH₂C(O)N(H)—. In embodiments of the formulae above, L⁷ is —CH₂N(H)C(O)—. In embodiments of the formulae above, L⁷ is —CH₂CH₂N(H)C(O)—.

The individual embodiments herein below, or combinations thereof, (e.g., embodiments of s1, s2, or s3) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, s1 is 1. In embodiments of the formulae above, s1 is 2. In embodiments of the formulae above, s1 is 3. In embodiments of the formulae above, s1 is 4. In embodiments of the formulae above, s1 is 5. In embodiments of the formulae above, s1 is 6. In embodiments of the formulae above, s2 is 1. In embodiments of the formulae above, s2 is 2. In embodiments of the formulae above, s2 is 3. In embodiments of the formulae above, s3 is 1. In embodiments of the formulae above, s3 is 2. In embodiments of the formulae above, s3 is 3. In embodiments of the formulae above, s2 is 0 (i.e., W² is a bond).

In some embodiments of Formula (IV-3), (IV-4), (IV), (IVa), or (IV′), s1 is 1 and s2 is 2. In some embodiments of Formula (IV) or (IV′), s1 is 1 and s2 is 3. In some embodiments of Formula (IV-3), (IV-4), (IV), (IVa), or (IV′), s1 is 2 and s2 is 1. In some embodiments of Formula (IV-3), (IV-4), (IV), (IVa), or (IV′), s1 is 2 and s2 is 2. In some embodiments of Formula (IV-3), (IV-4), (IV), (IVa), or (IV′), s1 is 2 and s2 is 3. In some embodiments of Formula (IV-3), (IV-4), (IV), (IVa), or (IV′), s1 is 3 and s2 is 1. In some embodiments of Formula (IV-3), (IV-4), (IV), (IVa), or (IV′), s1 is 3 and s2 is 2. In some embodiments of Formula (IV-3), (IV-4), (IV), (IVa), or (IV′), s1 is 3 and s2 is 3. In some embodiments of Formula (IV-3), (IV-4), (IV), (IVa), or (IV′), s1 is an integer from 2 to 6 and s2 is an integer from 1 to 3. In some embodiments of Formula (IV-3), (IV-4), (IV), (IVa), or (IV′), s1 is an integer from 1 to 6 and s2 is an integer from 2 to 3.

The individual embodiments herein below, or combinations thereof, (e.g., embodiments of L¹ or L^{1b}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, L¹ is a bond. In embodiments of the formulae above, L¹ is C₁-C₄alkyl. In embodiments of the formulae above, L¹ is C₂-C₄alkenyl. In embodiments of the formulae above, L¹ is C₂-C₄alkynyl. In embodiments of the formulae above, L¹ is —O—. In embodiments of the formulae above, L¹ is —N(R¹⁴)—. In embodiments of the formulae above, L¹ is —C(O)—. In embodiments of the formulae above, L¹ is —N(R¹⁴)C(O)—. In embodiments of the formulae above, L¹ is —C(O)N(R¹⁴)—. In embodiments of the formulae above, L¹ is —S—. In embodiments of the formulae above, L¹ is —S(O)₂—. In embodiments of the formulae above, L¹ is —S(O)—. In embodiments of the formulae above, L¹ is —S(O)₂N(R¹⁴)—. In embodiments of the formulae above, L¹ is —S(O)N(R¹⁴)—. In embodiments of the formulae above, L¹ is —N(R¹⁴)S(O)—. In embodiments of the formulae above, L¹ is —N(R¹⁴)S(O)₂—. In embodiments of the formulae above, L¹ is —OCON(R¹⁴)—. In embodiments of the formulae above, L¹ is —N(R¹⁴)C(O)O—. In embodiments of the formulae above, L¹ is N(R^{1e}). In embodiments of the formulae above, L¹ is C(O)N(R^{1c}). In embodiments of the formulae above, L¹ is S(O)₂N(R^{1c}). In embodiments of the formulae above, L¹ is S(O)N(R^{1c}). In embodiments of the formulae above, L¹ is C(R^{1f}(R^{1g})O. In embodiments of the formulae above, L¹ is C(R^{1f}(R^{1g})N(R^{1e}). In embodiments of the formulae above, L¹ is C(R^{1f}(R^{1g}).

In embodiments of the formulae above, Lib is a bond. In embodiments of the formulae above, Lib is C₁-C₄alkyl. In embodiments of the formulae above, Lib is C₂-C₄alkenyl. In embodiments of the formulae above, Lib is C₂-C₄alkynyl. In embodiments of the formulae above, Lib is —C(O)—. In embodiments of the formulae above, Lib is —C(O)N(R¹⁴)—. In embodiments of the formulae above, Lib is C(O)N(R^{1c}). In embodiments of the formulae above, Lib is C(R^{1f}(R^{1g})O. In embodiments of the formulae above, Lib is C(R^{1f}(R^{1g})N(R^{1c}). In embodiments of the formulae above, Lib is C(R^{1f}(R^{1g}).

The individual embodiments herein below, or combinations thereof, (e.g., embodiments of R¹⁹) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;

In embodiments of the formulae above, R¹⁹ is selected from a bicyclic C₄₋₁₂cycloalkyl, bicyclic C₂₋₁₁heterocycloalkyl, bicyclic C₇₋₁₂aryl, and bicyclic C₂₋₁₂heteroaryl, wherein the C₄₋₁₂cycloalkyl, bicyclic C₂₋₁₁heterocycloalkyl, bicyclic C₇₋₁₂aryl, and bicyclic C₂₋₁₂heteroaryl are optionally substituted with one, two, three, four, five, six, or seven R¹ⁱ.

In embodiments of the formulae above, R¹⁹ is selected from a bridged bicyclic C₄₋₁₂cycloalkyl, bridged bicyclic C₂₋₁₁heterocycloalkyl, bridged bicyclic C₇₋₁₂aryl, and bridged bicyclic C₂₋₁₂heteroaryl, wherein the bridged bicyclic C₄₋₁₂cycloalkyl, bridged bicyclic C₂₋₁₁heterocycloalkyl, bridged bicyclic C₇₋₁₂aryl, and bridged bicyclic C₂₋₁₂heteroaryl are optionally substituted with one, two, three, four, five, six, or seven R¹ⁱ.

In embodiments of the formulae above, R¹⁹ is selected from a fused bicyclic C₄₋₁₂cycloalkyl, fused bicyclic C₂₋₁₁heterocycloalkyl, fused bicyclic C₇₋₁₂aryl, and fused bicyclic C₂₋₁₂heteroaryl, wherein the fused bicyclic C₄₋₁₂cycloalkyl, fused bicyclic C₂₋₁₂heterocycloalkyl, fused bicyclic C₇₋₁₂aryl, and fused bicyclic C₂₋₁₂heteroaryl are optionally substituted with one, two, three, four, five, six, or seven R¹ⁱ.

In embodiments of the formulae above, R¹⁹ is a C₃₋₁₂cycloalkyl optionally substituted with one, two, three, four, five, six, or seven R¹ⁱ. In embodiments of the formulae above, R¹⁹ is a C₂₋₁₁heterocycloalkyl optionally substituted with one, two, three, four, five, six, or seven R¹ⁱ. In embodiments of the formulae above, R¹⁹ is a C₆₋₁₂aryl optionally substituted with one, two, three, four, five, six, or seven R¹ⁱ. In embodiments of the formulae above, R¹⁹ is a C₂₋₁₂heteroaryl optionally substituted with one, two, three, four, five, six, or seven R₁ᵢ. In embodiments of the formulae above, R¹⁹ is a C₃₋₁₂cycloalkyl. In embodiments of the formulae above, R¹⁹ is a C₂₋₁₁heterocycloalkyl. In embodiments of the formulae above, R¹⁹ is a C₆₋₁₂aryl. In embodiments of the formulae above, R¹⁹ is a C₂₋₁₂heteroaryl. In embodiments of the formulae above, R¹⁹ is a monocyclic C₃₋₉cycloalkyl optionally substituted with one, two, three, four, five, six, or seven R¹ⁱ. In embodiments of the formulae above, R¹⁹ is a monocyclic C₁₋₈heterocycloalkyl optionally substituted with one, two, three, four, five, six, or seven R¹ⁱ. In embodiments of the formulae above, R¹⁹ is a monocyclic phenyl optionally substituted with one, two, three, four, or five R¹ⁱ. In embodiments of the formulae above, R¹⁹ is a monocyclic C₁₋₅heteroaryl optionally substituted with one, two, three, four, or five R¹ⁱ. In embodiments of the formulae above, R¹⁹ is a spirocyclic C₅₋₁₂cycloalkyl optionally substituted with one, two, three, four, five, six, or seven R¹ⁱ. In embodiments of the formulae above, R¹⁹ is a spirocyclic C₂₋₁₁heterocycloalkyl optionally substituted with one, two, three, four, five, six, or seven R¹ⁱ. In embodiments of the formulae above, R¹⁹ is a fused C₄₋₁₂cycloalkyl optionally substituted with one, two, three, four, five, six, or seven R¹ⁱ. In embodiments of the formulae above, R¹⁹ is a fused C₂₋₁₁heterocycloalkyl optionally substituted with one, two, three, four, five, six, or seven R¹ⁱ. In embodiments of the formulae above, R¹⁹ is a fused C₆₋₁₂aryl, optionally substituted with one, two, three, four, five, six, or seven R¹ⁱ. In embodiments of the formulae above, R¹⁹ is a fused 5 to 12 membered heteroaryl optionally substituted with one, two, three, four, five, six, or seven R¹ⁱ.

In embodiments of the formulae above, R¹⁹ is:

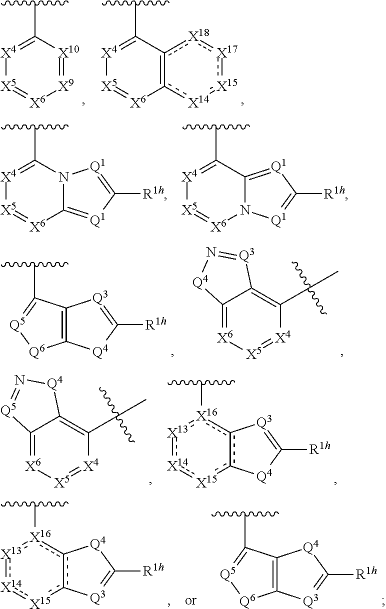

Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, X⁸ are independently selected from a C(O), C(R^{1a}), N, C(R^{1a})(R^{1b}), and N(R^{1c});
X¹⁶ are independently selected from C, N, and C(R^{1a});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₆heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R¹¹, and R^{id} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments of the formulae above, R¹⁹ is:

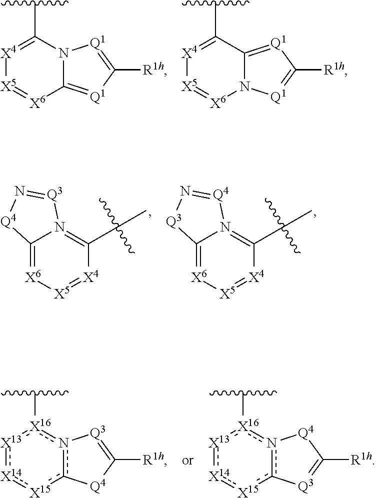

The individual embodiments herein below, or combinations thereof, (e.g., embodiments of Q¹, Q³, Q⁵, Q⁴, Q⁶, X⁴, X⁵, X⁶, X⁹, X¹⁰, X¹³, X¹⁴, X¹⁵, X¹⁶, X¹⁷, or X¹⁸) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, Q¹ is N. In embodiments of the formulae above, Q¹ is C(R^{1d}). In embodiments of the formulae above, Q³ is N. In embodiments of the formulae above, Q³ is C(R^{1d}). In embodiments of the formulae above, Q⁵ is N. In embodiments of the formulae above, Q⁵ is C(R^{1d}).

In embodiments of the formulae above, Q⁴ is O. In embodiments of the formulae above, Q⁴ is S. In embodiments of the formulae above, Q⁴ is C(R^{1a})(R^{1b}). In embodiments of the formulae above, Q⁴ is N(R^{1c}). In embodiments of the formulae above, Q⁶ is O. In embodiments of the formulae above, Q⁶ is S. In embodiments of the formulae above, Q⁶ is C(R^{1a})(R^{1b}). In embodiments of the formulae above, Q⁶ is N(R^{1c}).

In embodiments of the formulae above, X⁴ is C(R^{1a}). In embodiments of the formulae above, X⁴ is N. In embodiments of the formulae above, X⁵ is C(R^{1a}). In embodiments of the formulae above, X⁵ is N. In embodiments of the formulae above, X⁶ is C(R^{1a}). In embodiments of the formulae above, X⁶ is N. In embodiments of the formulae above, X⁹ is C(R^{1a}). In embodiments of the formulae above, X⁹ is N. In embodiments of the formulae above, X¹⁰ is C(R^{1a}). In embodiments of the formulae above, X¹⁰ is N.

In embodiments of the formulae above, X¹³ is a bond. In embodiments of the formulae above, X¹³ is C(R^{1a}). In embodiments of the formulae above, X¹³ is N. In embodiments of the formulae above, X¹³ is C(O). In embodiments of the formulae above, X¹³ is C(R^{1a})(R^{1b}). In embodiments of the formulae above, X¹³ is C(O)C(R^{1a})(R^{1b}). In embodiments of the formulae above, X¹³ is C(R^{1a})(R^{1b})C(R^{1a})(R^{1b}). In embodiments of the formulae above, X¹³ is C(R^{1a})(R^{1b})N(R^{1c}). In embodiments of the formulae above, X¹³ is N(R^{1c}).

In embodiments of the formulae above, X¹⁴ is C(R^{1a}). In embodiments of the formulae above, X¹⁴ is N. In embodiments of the formulae above, X¹⁴ is C(O). In embodiments of the formulae above, X¹⁴ is C(R^{1a})(R^{1b}). In embodiments of the formulae above, X¹⁴ is N(R^{1c}). In embodiments of the formulae above, X¹⁵ is C(R^{1a}). In embodiments of the formulae above, X¹⁵ is N. In embodiments of the formulae above, X¹⁵ is C(O). In embodiments of the formulae above, X¹⁵ is C(R^{1a})(R^{1b}). In embodiments of the formulae above, X¹⁵ is N(R^{1c}). In embodiments of the formulae above, X¹⁷ is C(R^{1a}). In embodiments of the formulae above, X¹⁷ is N. In embodiments of the formulae above, X¹⁷ is C(O). In embodiments of the formulae above, X¹⁷ is C(R^{1a})(R^{1b}). In embodiments of the formulae above, X¹⁷ is N(R^{1c}). In embodiments of the formulae above, X¹¹ is C(R^{1a}). In embodiments of the formulae above, X¹⁸ is N. In embodiments of the formulae above, X¹⁸ is C(O). In embodiments of the formulae above, X¹⁸ is C(R^{1a})(R^{1b}). In embodiments of the formulae above, X¹⁸ is N(R^{1c}).

In embodiments of the formulae above, X¹⁶ is C. In embodiments of the formulae above, X¹⁶ is N. In embodiments of the formulae above, X¹⁶ is C(R^{1a}).

The individual embodiments herein below, or combinations thereof, (e.g., embodiments of R^{1a}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1a} is independently hydrogen. In embodiments of the formulae above, each R^{1a} is independently halogen. In embodiments of the formulae above, each R^{1a} is independently oxo. In embodiments of the formulae above, each R^{1a} is independently —CN. In embodiments of the formulae above, each R^{1a} is independently C₁₋₆alkyl. In embodiments of the formulae above, each R^{1a} is independently C₂₋₆alkenyl. In embodiments of the formulae above, each R^{1a} is independently C₂₋₆alkynyl. In embodiments of the formulae above, each R^{1a} is independently C₃₋₁₀cycloalkyl. In embodiments of the formulae above, each R^{1a} is independently C₂₋₉heterocycloalkyl. In embodiments of the formulae above, each R^{1a} is independently C₆₋₁₀aryl. In embodiments of the formulae above, each R^{1a} is independently C₁₋₉heteroaryl. In embodiments of the formulae above, each R^{1a} is independently —OR¹². In embodiments of the formulae above, each R^{1a} is independently —SR¹². In embodiments of the formulae above, each R^{1a} is independently —N(R¹²)(R¹³). In embodiments of the formulae above, each R^{1a} is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of the formulae above, each R^{1a} is independently C₁₋₆alkyl substituted with one, two, or three R²⁰ⁱ. In embodiments of the formulae above, each R^{1a} is independently C₂₋₆alkenyl substituted with one, two, or three R²⁰ⁱ. In embodiments of the formulae above, each R^{1a} is independently C₂₋₆alkynyl substituted with one, two, or three R²⁰ⁱ. In embodiments of the formulae above, each R^{1a} is independently C₃₋₁₀cycloalkyl substituted with one, two, or three R²⁰ⁱ. In embodiments of the formulae above, each R^{1a} is independently C₂₋₉heterocycloalkyl substituted with one, two, or three R²⁰ⁱ. In embodiments of the formulae above, each R^{1a} is independently C₆₋₁₀aryl substituted with one, two, or three R²⁰ⁱ. In embodiments of the formulae above, each R^{1a} is independently C₁₋₉heteroaryl substituted with one, two, or three R². In embodiments of the formulae above, R^{1a} is independently halogen. In embodiments of the formulae above, R^{1a} is independently F. In embodiments of the formulae above, R^{1a} is independently Cl. In embodiments of the formulae above, R^{1a} is independently Br. In embodiments of the formulae above, R^{1a} is independently I. In embodiments of the formulae above, R^{1a} is independently R^{1a} is independently oxo. In embodiments of the formulae above, R^{1a} is independently —CN. In embodiments of the formulae above, R^{1a} is independently C₁₋₆alkyl. In embodiments of the formulae above, R^{1a} is independently methyl. In embodiments of the formulae above, R^{1a} is independently ethyl. In embodiments of the formulae above, R^{1a} is independently isopropyl. In embodiments of the formulae above, R^{1a} is independently C₂₋₆alkenyl. In embodiments of the formulae above, R^{1a} is independently C₂₋₆alkynyl. In embodiments of the formulae above, R^{1a} is independently C₁₋₆haloalkyl. In embodiments of the formulae above, R^{1a} is independently —CF₃. In embodiments of the formulae above, R^{1a} is independently C₃₋₁₂cycloalkyl. In embodiments of the formulae above, R^{1a} is independently C₂₋₁₁heterocycloalkyl. In embodiments of the formulae above, R^{1a} is independently C₆₋₁₂aryl. In embodiments of the formulae above, R^{1a} is independently C₁₋₁₁heteroaryl. In embodiments of the formulae above, R^{1a} is independently —OH. In embodiments of the formulae above, R^{1a} is independently —OCH₃. In embodiments of the formulae above, R^{1a} is independently —SH. In embodiments of the formulae above, R^{1a} is independently —SCH₃. In embodiments of the formulae above, R^{1a} is independently —N(CH₃)₂. In embodiments of the formulae above, R^{1a} is independently —N(H)₂. In embodiments of the formulae above, R^{1a} is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H, —CH₂S(O)₂CH₃, —CH₂S(O)₂N(CH₃)₂, and —CH₂S(O)₂N(H)₂.

The individual embodiments herein below, or combinations thereof, (e.g., embodiments of R^{1b}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1b} is independently hydrogen. In embodiments of the formulae above, each R^{1b} is independently halogen. In embodiments of the formulae above, each R^{1b} is independently oxo. In embodiments of the formulae above, each R^{1b} is independently —CN. In embodiments of the formulae above, each R^{1b} is independently C₁₋₆alkyl. In embodiments of the formulae above, each R^{1b} is independently C₂₋₆alkenyl. In embodiments of the formulae above, each R^{1b} is independently C₂₋₆alkynyl. In embodiments of the formulae above, each R^{1b} is independently C₃₋₁₀cycloalkyl. In embodiments of the formulae above, each R^{1b} is independently C₂₋₉heterocycloalkyl. In embodiments of the formulae above, each R^{1b} is independently C₆₋₁₀aryl. In embodiments of the formulae above, each R^{1b} is independently C₁₋₉heteroaryl. In embodiments of the formulae above, each R^{1b} is independently —OR¹². In embodiments of the formulae above, each R^{1b} is independently —SR¹². In embodiments of the formulae above, each R^{1b} is independently —N(R¹²)(R¹³). In embodiments of the formulae above, each R^{1b} is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of the formulae above, each R^{1b} is independently C₁₋₆alkyl substituted with one, two, or three R²⁰ⁱ. In embodiments of the formulae above, each R^{1b} is independently C₂₋₆alkenyl substituted with one, two, or three R²⁰ⁱ. In embodiments of the formulae above, each R^{1b} is independently C₂₋₆alkynyl substituted with one, two, or three R²⁰ⁱ. In embodiments of the formulae above, each R^{1b} is independently C₃₋₁₀cycloalkyl substituted with one, two, or three R²⁰ⁱ. In embodiments of the formulae above, each R^{1b} is independently C₂₋₉heterocycloalkyl substituted with one, two, or three R²⁰ⁱ. In embodiments of the formulae above, each R^{1b} is independently C₆₋₁₀aryl substituted with one, two, or three R²⁰ⁱ. In embodiments of the formulae above, each R^{1b} is independently C₁₋₉heteroaryl substituted with one, two, or three R²⁰ⁱ. In embodiments of the formulae above, R^{1b} is independently halogen. In embodiments of the formulae above, R^{1b} is independently F. In embodiments of the formulae above, R^{1b} is independently Cl. In embodiments of the formulae above, R^{1b} is independently Br. In embodiments of the formulae above, R^{1b} is independently C₁₋₆alkyl. In embodiments of the formulae above, R^{1b} is independently methyl. In embodiments of the formulae above, R^{1b} is independently ethyl. In embodiments of the formulae above, R^{1b} is independently isopropyl. In embodiments of the formulae above, R^{1b} is independently C₂₋₆alkenyl. In embodiments of the formulae above, R^{1b} is independently C₂₋₆alkynyl. In embodiments of the formulae above, R^{1b} is independently C₁₋₆haloalkyl. In embodiments of the formulae above, R^{1b} is independently —CF₃. In embodiments of the formulae above, R^{1b} is independently C₃₋₁₂cycloalkyl. In embodiments of the formulae above, R^{1b} is independently C₂₋₁₁heterocycloalkyl. In embodiments of the formulae above, R^{1b} is independently C₆₋₁₂aryl. In embodiments of the formulae above, R^{1b} is independently C₁₋₁₁heteroaryl. In embodiments of the formulae above, R^{1b} is independently —OH. In embodiments of the formulae above, R^{1b} is independently —OCH₃. In embodiments of the formulae above, R^{1b} is independently —SH. In embodiments of the formulae above, R^{1b} is independently —SCH₃. In embodiments of the formulae above, R^{1b} is independently —N(CH₃)₂. In embodiments of the formulae above, R^{1b} is independently —N(H)₂. In embodiments of the formulae above, R^{1b} is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H, —CH₂S(O)₂CH₃, —CH₂S(O)₂N(CH₃)₂, and —CH₂S(O)₂N(H)₂.

The individual embodiments herein below, or combinations thereof, (e.g., embodiments of R^{1d}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{id} is independently hydrogen. In embodiments of the formulae above, each R^{1d} is independently halogen. In embodiments of the formulae above, each R^{1d} is independently oxo. In embodiments of the formulae above, each R^{1d} is independently —CN. In embodiments of the formulae above, each R^{1d} is independently C₁₋₆alkyl. In embodiments of the formulae above, each R^{1d} is independently C₂₋₆alkenyl. In embodiments of the formulae above, each R^{1d} is independently C₂₋₆alkynyl. In embodiments of the formulae above, each R^{1d} is independently C₃₋₁₀cycloalkyl. In embodiments of the formulae above, each R^{1d} is independently C₂₋₉heterocycloalkyl. In embodiments of the formulae above, each R^{1d} is independently C₆₋₁₀aryl. In embodiments of the formulae above, each R^{1d} is independently C₁₋₉heteroaryl. In embodiments of the formulae above, each R^{1d} is independently —OR¹². In embodiments of the formulae above, each R^{1d} is independently —SR¹². In embodiments of the formulae above, each R^{1d} is independently —N(R¹²)(R¹³). In embodiments of the formulae above, each R^{1d} is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of the formulae above, each R^{1d} is independently C₁₋₆alkyl substituted with one, two, or three R²⁰ⁱ. In embodiments of the formulae above, each R^{1d} is independently C₂₋₆alkenyl substituted with one, two, or three R²⁰ⁱ. In embodiments of the formulae above, each R^{1d} is independently C₂₋₆alkynyl substituted with one, two, or three R². In embodiments of the formulae above, each R^{1d} is independently C₃₋₁₀cycloalkyl substituted with one, two, or three R². In embodiments of the formulae above, each R^{1d} is independently C₂₋₉heterocycloalkyl substituted with one, two, or three R². In embodiments of the formulae above, each R^{1d} is independently C₆₋₁₀aryl substituted with one, two, or three R²⁰ⁱ. In embodiments of the formulae above, each R^{1d} is independently C₁₋₉heteroaryl substituted with one, two, or three R²⁰ⁱ. In embodiments of the formulae above, R^{1d} is independently halogen. In embodiments of the formulae above, R^{1d} is independently F. In embodiments of the formulae above, R^{1d} is independently Cl. In embodiments of the formulae above, R^{1d} is independently Br. In embodiments of the formulae above, R^{1d} is independently I. In embodiments of the formulae above, R^{1d} is independently C₁₋₆alkyl. In embodiments of the formulae above, R^{1d} is independently methyl. In embodiments of the formulae above, R^{1d} is independently ethyl. In embodiments of the formulae above, R^{1d} is independently isopropyl. In embodiments of the formulae above, R^{1d} is independently C₂₋₆alkenyl. In embodiments of the formulae above, R^{1d} is independently C₂₋₆alkynyl. In embodiments of the formulae above, R^{1d} is independently C₁₋₆haloalkyl. In embodiments of the formulae above, R^{1d} is independently —CF₃. In embodiments of the formulae above, R^{1d} is independently C₃₋₁₂cycloalkyl. In embodiments of the formulae above, R^{1d} is independently C₂₋₁₁heterocycloalkyl. In embodiments of the formulae above, R^{1d} is independently C₆₋₁₂aryl. In embodiments of the formulae above, R^{1d} is independently C₁₋₁₁heteroaryl. In embodiments of the formulae above, R^{1d} is independently —OH. In embodiments of the formulae above, R^{1d} is independently —OCH₃. In embodiments of the formulae above, R^{1d} is independently —SH. In embodiments of the formulae above, R^{1d} is independently —SCH₃. In embodiments of the formulae above, R^{1d} is independently —N(CH₃)₂. In embodiments of the formulae above, R^{id} is independently —N(H)₂. In embodiments of the formulae above, R^{id} is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H, —CH₂S(O)₂CH₃, —CH₂S(O)₂N(CH₃)₂, and —CH₂S(O)₂N(H)₂.

The individual embodiments herein below, or combinations thereof, (e.g., embodiments of R^{1e}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1e} is independently hydrogen. In embodiments of the formulae above, each R^{1e} is independently halogen. In embodiments of the formulae above, each R^{1e} is independently oxo. In embodiments of the formulae above, each R^{1e} is independently —CN. In embodiments of the formulae above, each R^{1e} is independently C₁₋₆alkyl. In embodiments of the formulae above, each R^{1e} is independently C₂₋₆alkenyl. In embodiments of the formulae above, each R^{1e} is independently C₂₋₆alkynyl. In embodiments of the formulae above, each R^{1e} is independently C₃₋₁₀cycloalkyl. In embodiments of the formulae above, each R^{1e} is independently C₂₋₉heterocycloalkyl. In embodiments of the formulae above, each R^{1e} is independently C₆₋₁₀aryl. In embodiments of the formulae above, each R^{1e} is independently C₁₋₉heteroaryl. In embodiments of the formulae above, each R^{1e} is independently —OR¹². In embodiments of the formulae above, each R^{1e} is independently —SR¹². In embodiments of the formulae above, each R^{1e} is independently —N(R¹²)(R¹³). In embodiments of the formulae above, each R^{1e} is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of the formulae above, each R^{1e} is independently C₁₋₆alkyl substituted with one, two, or three R²⁰ⁱ. In embodiments of the formulae above, each R^{1e} is independently C₂₋₆alkenyl substituted with one, two, or three R²⁰ⁱ. In embodiments of the formulae above, each R^{1e} is independently C₂₋₆alkynyl substituted with one, two, or three R²⁰ⁱ. In embodiments of the formulae above, each R^{1e} is independently C₃₋₁₀cycloalkyl substituted with one, two, or three R²⁰ⁱ. In embodiments of the formulae above, each R^{1e} is independently C₂₋₉heterocycloalkyl substituted with one, two, or three R²⁰ⁱ. In embodiments of the formulae above, each R^{1e} is independently C₆₋₁₀aryl substituted with one, two, or three R²⁰ⁱ. In embodiments of the formulae above, each R^{1e} is independently C₁₋₉heteroaryl substituted with one, two, or three R². In embodiments of the formulae above, R^{1e} is independently halogen. In embodiments of the formulae above, R^{1e} is independently F. In embodiments of the formulae above, R^{1e} is independently Cl. In embodiments of the formulae above, R^{1e} is independently Br. In embodiments of the formulae above, R^{1e} is independently I. In embodiments of the formulae above, R^{1e} is independently C₁₋₆alkyl. In embodiments of the formulae above, R^{1e} is independently methyl. In embodiments of the formulae above, R^{1e} is independently ethyl. In embodiments of the formulae above, R^{1e} is independently isopropyl. In embodiments of the formulae above, R^{1e} is independently C₂₋₆alkenyl. In embodiments of the formulae above, R^{1e} is independently C₂₋₆alkynyl. In embodiments of the formulae above, R^{1e} is independently C₁₋₆haloalkyl. In embodiments of the formulae above, R^{1e} is independently —CF₃. In embodiments of the formulae above, R^{1e} is independently C₃₋₁₂cycloalkyl. In embodiments of the formulae above, R^{1e} is independently C₂₋₁₁heterocycloalkyl. In embodiments of the formulae above, R^{1e} is independently C₆₋₁₂aryl. In embodiments of the formulae above, R^{1e} is independently C₁₋₁₁heteroaryl. In embodiments of the formulae above, R^{1e} is independently —OH. In embodiments of the formulae above, R^{1e} is independently —OCH₃. In embodiments of the formulae above, R^{1e} is independently —SH. In embodiments of the formulae above, R^{1e} is independently —SCH3. In embodiments of the formulae above, R^{1e} is independently —N(CH₃)₂. In embodiments of the formulae above, R^{1e} is independently —N(H)₂. In embodiments of the formulae above, R^{1e} is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H, —CH₂S(O)₂CH₃, —CH₂S(O)₂N(CH₃)₂, and —CH₂S(O)₂N(H)₂.

The individual embodiments herein below, or combinations thereof, (e.g., embodiments of R¹) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1f} is independently hydrogen. In embodiments of the formulae above, each R^{1f} is independently halogen. In embodiments of the formulae above, each R^{1f} is independently oxo. In embodiments of the formulae above, each R^{1f} is independently —CN. In embodiments of the formulae above, each R^{1f} is independently C₁₋₆alkyl. In embodiments of the formulae above, each R^{1f} is independently C₂₋₆alkenyl. In embodiments of the formulae above, each R^{1f} is independently C₂₋₆alkynyl. In embodiments of the formulae above, each R^{1f} is independently C₃₋₁₀cycloalkyl. In embodiments of the formulae above, each R^{1f} is independently C₂₋₉heterocycloalkyl. In embodiments of the formulae above, each R^{1f} is independently C₆₋₁₀aryl. In embodiments of the formulae above, each R^{1f} is independently C₁₋₉heteroaryl. In embodiments of the formulae above, each R^{1f} is independently —OR¹². In embodiments of the formulae above, each R^{1f} is independently —SR¹². In embodiments of the formulae above, each R^{1f} is independently —N(R¹²)(R¹³). In embodiments of the formulae above, each R^{1f} is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of the formulae above, each R^{1f} is independently C₁₋₆alkyl substituted with one, two, or three R²⁰ⁱ. In embodiments of the formulae above, each R^{1f} is independently C₂₋₆alkenyl substituted with one, two, or three R²⁰ⁱ. In embodiments of the formulae above, each R^{1f} is independently C₂₋₆alkynyl substituted with one, two, or three R^{2m}. In embodiments of the formulae above, each R^{1f} is independently C₃₋₁₀cycloalkyl substituted with one, two, or three R². In embodiments of the formulae above, each R^{1f} is independently C₂₋₉heterocycloalkyl substituted with one, two, or three R²⁰. In embodiments of the formulae above, each R^{1f} is independently C₆₋₁₀aryl substituted with one, two, or three R²⁰ⁱ. In embodiments of the formulae above, each R^{1f} is independently C₁₋₉ heteroaryl substituted with one, two, or three R²⁰ⁱ. In embodiments of the formulae above, R^{1f} is independently halogen. In embodiments of the formulae above, R^{1f} is independently F. In embodiments of the formulae above, R^{1f} is independently Cl. In embodiments of the formulae above, R^{1f} is independently Br. In embodiments of the formulae above, R^{1f} is independently I. In embodiments of the formulae above, R^{1f} is independently C₁₋₆alkyl. In embodiments of the formulae above, R^{1f} is independently methyl. In embodiments of the formulae above, R^{1f} is independently ethyl. In embodiments of the formulae above, R^{1f} is independently isopropyl. In embodiments of the formulae above, R^{1f} is independently C₂₋₆alkenyl. In embodiments of the formulae above, R^{1f} is independently C₂₋₆alkynyl. In embodiments of the formulae above, R^{1f} is independently C₁₋₆haloalkyl. In embodiments of the formulae above, R^{1f} is independently —CF₃. In embodiments of the formulae above, R^{1f} is independently C₃₋₁₂cycloalkyl. In embodiments of the formulae above, R^{1f} is independently C₂₋₁₁heterocycloalkyl. In embodiments of the formulae above, R^{1f} is independently C₆₋₁₂aryl. In embodiments of the formulae above, R^{1f} is independently C₁₋₁₁heteroaryl. In embodiments of the formulae above, R^{1f} is independently —OH. In embodiments of the formulae above, R^{1f} is independently —OCH₃. In embodiments of the formulae above, R^{1f} is independently —SH. In embodiments of the formulae above, R^{1f} is independently —SCH3. In embodiments of the formulae above, R^{1f} is independently —N(CH₃)₂. In embodiments of the formulae above, R^{1f} is independently —N(H)₂. In embodiments of the formulae above, R^{1f} is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H. —CH₂S(O)₂CH₃, —CH₂S(O)₂N(CH₃)₂, and —CH₂S(O)₂N(H)₂.

The individual embodiments herein below, or combinations thereof, (e.g., embodiments of R¹⁹) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1g} is independently hydrogen. In embodiments of the formulae above, each R¹⁹ is independently halogen. In embodiments of the formulae above, each R¹⁹ is independently oxo. In embodiments of the formulae above, each R¹⁹ is independently —CN. In embodiments of the formulae above, each R¹⁹ is independently C₁₋₆alkyl. In embodiments of the formulae above, each R¹⁹ is independently C₂₋₆alkenyl. In embodiments of the formulae above, each R¹⁹ is independently C₂₋₆alkynyl. In embodiments of the formulae above, each R¹⁹ is independently C₃₋₁₀cycloalkyl. In embodiments of the formulae above, each R¹⁹ is independently C₂₋₉heterocycloalkyl. In embodiments of the formulae above, each R¹⁹ is independently C₆₋₁₀aryl. In embodiments of the formulae above, each R¹⁹ is independently C₁₋₉heteroaryl. In embodiments of the formulae above, each R¹⁹ is independently —OR¹². In embodiments of the formulae above, each R¹⁹ is independently —SR¹². In embodiments of the formulae above, each R¹⁹ is independently —N(R¹²)(R¹³). In embodiments of the formulae above, each R¹⁹ is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(o)₂R⁵, —S(o)₂N(R¹²)(R¹³), —S(═O)(═NH)N(R¹²)(R¹³), —CH₂C(O)N(R¹²)(R¹³), —CH₂N(R¹⁴)C(O)R⁵, —CH₂S(O)₂R⁵, and —CH₂S(o)₂N(R¹²)(R¹³). In embodiments of the formulae above, each R¹⁹ is independently C₁₋₆alkyl substituted with one, two, or three R²⁰ⁱ. In embodiments of the formulae above, each R¹⁹ is independently C₂₋₆alkenyl substituted with one, two, or three R²⁰ⁱ. In embodiments of the formulae above, each R¹⁹ is independently C₂₋₆alkynyl substituted with one, two, or three R²⁰ⁱ. In embodiments of the formulae above, each R¹⁹ is independently C₃₋₁₀cycloalkyl substituted with one, two, or three R²⁰ⁱ. In embodiments of the formulae above, each R¹⁹ is independently C₂₋₉heterocycloalkyl substituted with one, two, or three R²¹. In embodiments of the formulae above, each R¹⁹ is independently C₆₋₁₀aryl substituted with one, two, or three R²⁰ⁱ. In embodiments of the formulae above, each R¹⁹ is independently C₁₋₉heteroaryl substituted with one, two, or three R²¹. In embodiments of the formulae above, R¹⁹ is independently halogen. In embodiments of the formulae above, R¹⁹ is independently F. In embodiments of the formulae above, R¹⁹ is independently Cl. In embodiments of the formulae above, R¹⁹ is independently Br. In embodiments of the formulae above, R¹⁹ is independently I. In embodiments of the formulae above, R¹⁹ is independently R¹⁹ is independently oxo. In embodiments of the formulae above, R¹⁹ is independently —CN. In embodiments of the formulae above, R¹⁹ is independently C₁₋₆alkyl. In embodiments of the formulae above, R¹⁹ is independently methyl. In embodiments of the formulae above, R¹⁹ is independently ethyl. In embodiments of the formulae above, R¹⁹ is independently isopropyl. In embodiments of the formulae above, R¹⁹ is independently C₂₋₆alkenyl. In embodiments of the formulae above, R¹⁹ is independently C₂₋₆alkynyl. In embodiments of the formulae above, R¹⁹ is independently C₁₋₆haloalkyl. In embodiments of the formulae above, R¹⁹ is independently —CF₃. In embodiments of the formulae above, R¹⁹ is independently C₃₋₁₂cycloalkyl. In embodiments of the formulae above, R¹⁹ is independently C₂₋₁₁heterocycloalkyl. In embodiments of the formulae above, R¹⁹ is independently C₆₋₁₂aryl. In embodiments of the formulae above, R¹⁹ is independently C₁₋₁₁heteroaryl. In embodiments of the formulae above, R¹⁹ is independently —OH. In embodiments of the formulae above, R¹⁹ is independently —OCH₃. In embodiments of the formulae above, R¹⁹ is independently —SH. In embodiments of the formulae above, R¹⁹ is independently —SCH₃. In embodiments of the formulae above, R¹⁹ is independently —N(CH₃)₂. In embodiments of the formulae above, R¹⁹ is independently —N(H)₂. In embodiments of the formulae above, R¹⁹ is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H, —CH₂S(O)₂CH₃, —CH₂S(O)₂N(CH₃)₂, and —CH₂S(O)₂N(H)₂.

The individual embodiments herein below, or combinations thereof, (e.g., embodiments of R^{1h}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1h} is independently hydrogen. In embodiments of the formulae above, each R^{1h} is independently halogen. In embodiments of the formulae above, each R^{1h} is independently oxo. In embodiments of the formulae above, each R^{1h} is independently —CN. In embodiments of the formulae above, each R^{1h} is independently C₁₋₆alkyl. In embodiments of the formulae above, each R^{1h} is independently C₂₋₆alkenyl. In embodiments of the formulae above, each R^{1h} is independently C₂₋₆alkynyl. In embodiments of the formulae above, each R^{1h} is independently C₃₋₁₀cycloalkyl. In embodiments of the formulae above, each R^{1h} is independently C₂₋₉heterocycloalkyl. In embodiments of the formulae above, each R^{1h} is independently C₆₋₁₀aryl. In embodiments of the formulae above, each R^{1h} is independently C₁₋₉heteroaryl. In embodiments of the formulae above, each R^{1h} is independently —OR¹². In embodiments of the formulae above, each R^{1h} is independently —SR¹². In embodiments of the formulae above, each R^{1h} is independently —N(R¹²)(R¹³). In embodiments of the formulae above, each R^{1h} is independently selected from —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of the formulae above, each R^{1h} is independently C₁₋₆alkyl substituted with one, two, or three R²⁰ⁱ. In embodiments of the formulae above, each R^{1h} is independently C₂₋₆alkenyl substituted with one, two, or three R²⁰ⁱ. In embodiments of the formulae above, each R^{1h} is independently C₂₋₆alkynyl substituted with one, two, or three R²⁰ⁱ. In embodiments of the formulae above, each R^{1h} is independently C₃₋₁₀cycloalkyl substituted with one, two, or three R²⁰ⁱ. In embodiments of the formulae above, each R^{1h} is independently C₂₋₉heterocycloalkyl substituted with one, two, or three R²⁰ⁱ. In embodiments of the formulae above, each R^{1h} is independently C₆₋₁₀aryl substituted with one, two, or three R²⁰ⁱ. In embodiments of the formulae above, each R^{1h} is independently C₁₋₉heteroaryl substituted with one, two, or three R²⁰ⁱ. In embodiments of the formulae above, R^{1h} is independently halogen. In embodiments of the formulae above, R^{1h} is independently F. In embodiments of the formulae above, R^{1h} is independently Cl. In embodiments of the formulae above, R^{1h} is independently Br. In embodiments of the formulae above, R^{1h} is independently I. In embodiments of the formulae above, R^{1h} is independently C₁₋₆alkyl. In embodiments of the formulae above, R^{1h} is independently methyl. In embodiments of the formulae above, R^{1h} is independently ethyl. In embodiments of the formulae above, R^{1h} is independently isopropyl. In embodiments of the formulae above, R^{1h} is independently C₂₋₆alkenyl. In embodiments of the formulae above, R^{1h} is independently C₂₋₆alkynyl. In embodiments of the formulae above, R^{1h} is independently C₁₋₆haloalkyl. In embodiments of the formulae above, R^{1h} is independently —CF₃. In embodiments of the formulae above, R^{1h} is independently C₃₋₁₂cycloalkyl. In embodiments of the formulae above, R^{1h} is independently C₂₋₁₁heterocycloalkyl. In embodiments of the formulae above, R^{1h} is independently C₆₋₁₂aryl. In embodiments of the formulae above, R^{1h} is independently C₁₋₁₁heteroaryl. In embodiments of the formulae above, R^{1h} is independently —OH. In embodiments of the formulae above, R^{1h} is independently —OCH₃. In embodiments of the formulae above, R^{1h} is independently —SH. In embodiments of the formulae above, R^{1h} is independently —SCH₃. In embodiments of the formulae above, R^{1h} is independently —N(CH₃)₂. In embodiments of the formulae above, R^{1h} is independently —N(H)₂. In embodiments of the formulae above, R^{1h} is independently selected from —C(O)OH, —C(O)OCH₃, —OC(O)N(H)₂, —OC(O)N(CH₃)₂, —N(H)C(O)N(CH₃)₂, —N(H)C(O)N(H)₂, —N(H)C(O)OH, —N(H)C(O)OCH₃, —N(H)S(O)₂CH₃, —C(O)CH₃, —C(O)H, —S(O)CH₃, —OC(O)CH₃, —OC(O)H, —C(O)N(CH₃)₂, —C(O)C(O)N(CH₃)₂, —N(H)C(O)H, —N(H)C(O)CH₃, —S(O)₂CH₃, —S(O)₂N(H)₂, —S(O)₂N(CH₃)₂, S(═O)(═NH)N(H)₂, S(═O)(═NH)N(CH₃)₂, —CH₂C(O)N(H)₂, —CH₂C(O)N(CH₃)₂, —CH₂N(H)C(O)H, —CH₂N(H)C(O)CH₃, —CH₂S(O)₂H., —CH₂S(O)₂CH₃, —CH₂S(O)₂N(CH₃)₂, and —CH₂S(O)₂N(H)₂.

The individual embodiments herein below, or combinations thereof, (e.g., embodiments of R^{1c}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, each R^{1c} is independently hydrogen. In embodiments of the formulae above, each R^{1c} is independently C₁₋₆alkyl. In embodiments of the formulae above, each R^{1c} is independently C₂₋₆alkenyl. In embodiments of the formulae above, each R^{1c} is independently C₂₋₆alkynyl. In embodiments of the formulae above, each R^{1c} is independently C₃₋₁₀cycloalkyl. In embodiments of the formulae above, each R^{1e} is independently C₂₋₉heterocycloalkyl. In embodiments of the formulae above, each R^{1c} is independently C₆₋₁₀aryl. In embodiments of the formulae above, each R^{1e} is independently C₁₋₉heteroaryl. In embodiments of the formulae above, each R^{1e} is independently C₁₋₆alkyl substituted with one, two, or three R²⁰ⁱ. In embodiments of the formulae above, each R^{1e} is independently C₂₋₆alkenyl substituted with one, two, or three R²⁰ⁱ. In embodiments of the formulae above, each R^{1e} is independently C₂₋₆alkynyl substituted with one, two, or three R²⁰ⁱ. In embodiments of the formulae above, each R^{1e} is independently C₃₋₁₀cycloalkyl substituted with one, two, or three R²⁰ⁱ. In embodiments of the formulae above, each R^{1c} is independently C₂₋₉heterocycloalkyl substituted with one, two, or three R²⁰ⁱ. In embodiments of the formulae above, each R^{1c} is independently C₆₋₁₀aryl substituted with one, two, or three R²⁰. In embodiments of the formulae above, each R^{1c} is independently C₁₋₉heteroaryl substituted with one, two, or three R²⁰. In embodiments of the formulae above, R^{1c} is independently C₁₋₆ alkyl. In embodiments of the formulae above, R^{1c} is independently methyl. In embodiments of the formulae above, R^{1e} is independently ethyl. In embodiments of the formulae above, R^{1e} is independently isopropyl. In embodiments of the formulae above, R^{1e} is independently C₂₋₆alkenyl. In embodiments of the formulae above, R^{1e} is independently C₂₋₆alkynyl. In embodiments of the formulae above, R^{1e} is independently C₁₋₆haloalkyl. In embodiments of the formulae above, R^{1e} is independently —CF₃. In embodiments of the formulae above, R^{1e} is independently C₃₋₁₂cycloalkyl. In embodiments of the formulae above, R^{1e} is independently C₂₋₁₁heterocycloalkyl. In embodiments of the formulae above, R^{1c} is independently C₆₋₁₂aryl. In embodiments of the formulae above, R^{1e} is independently C₁₋₁₁heteroaryl.

The individual embodiments herein below, or combinations thereof, (e.g., embodiments of R¹ⁱ) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R¹ⁱ is halogen. In embodiments of the formulae above, R^{1e} is —CN. In embodiments of the formulae above, R¹ⁱ is C₁₋₆alkyl. In embodiments of the formulae above, R^{1e} is C₂₋₆alkenyl. In embodiments of the formulae above, R¹ⁱ is C₂₋₆alkynyl. In embodiments of the formulae above, R^{1e} is C₃₋₁₀cycloalkyl. In embodiments of the formulae above, R¹ⁱ is C₂₋₉heterocycloalkyl. In embodiments of the formulae above, R¹ⁱ is C₆₋₁₀aryl. In embodiments of the formulae above, R¹ⁱ is C₁₋₉heteroaryl.

In embodiments of the formulae above, R¹ⁱ is C₁₋₆alkyl optionally substituted with one, two, or three R²⁰ⁱ. In embodiments of the formulae above, R¹ⁱ is C₂₋₆alkenyl optionally substituted with one, two, or three R²⁰ⁱ. In embodiments of the formulae above, R¹ⁱ is C₂₋₆alkynyl optionally substituted with one, two, or three R²⁰ⁱ. In embodiments of the formulae above, R¹ⁱ is C₃₋₁₀cycloalkyl optionally substituted with one, two, or three R²⁰ⁱ. In embodiments of the formulae above, R¹ⁱ is C₂₋₉heterocycloalkyl optionally substituted with one, two, or three R²⁰ⁱ. In embodiments of the formulae above, R¹ⁱ is C₆₋₁₀aryl optionally substituted with one, two, or three R²⁰ⁱ. In embodiments of the formulae above, R¹ⁱ is C₁₋₉heteroaryl optionally substituted with one, two, or three R²⁰ⁱ.

In embodiments of the formulae above, R¹ⁱ is —OR¹². In embodiments of the formulae above, R¹ⁱ is —SR¹². In embodiments of the formulae above, R¹ⁱ is —N(R¹²)(R¹³). In embodiments of the formulae above, R¹ⁱ is —C(O)OR¹². In embodiments of the formulae above, R¹ⁱ is —OC(O)N(R¹²)(R¹³). In embodiments of the formulae above, R¹ⁱ is —N(R¹⁴)C(O)N(R¹²)(R¹³). In embodiments of the formulae above, R¹ⁱ is —N(R¹⁴)C(O)OR¹⁵. In embodiments of the formulae above, R¹ⁱ is —N(R¹⁴)S(O)₂R¹⁵. In embodiments of the formulae above, R^{1e} is —C(O)R¹⁵. In embodiments of the formulae above, R^{1e} is —S(O)R¹⁵. In embodiments of the formulae above, R^{1e} is —OC(O)R¹⁵. In embodiments of the formulae above, R^{1e} is —C(O)N(R¹²)(R¹³). In embodiments of the formulae above, R¹ⁱ is —C(O)C(O)N(R¹²)(R¹³). In embodiments of the formulae above, R¹ⁱ is —N(R¹⁴)C(O)R¹⁵. In embodiments of the formulae above, R^{1e} is —S(O)₂R¹⁵. In embodiments of the formulae above, R^{1e} is —S(O)₂N(R¹²)(R¹³). In embodiments of the formulae above, R^{1e} is S(═O)(═NH)N(R¹²)(R¹³). In embodiments of the formulae above, R¹ⁱ is —CH₂C(O)N(R¹²)(R¹³). In embodiments of the formulae above, R¹ⁱ is —CH₂N(R¹⁴)C(O)R¹⁵. In embodiments of the formulae above, R¹ⁱ is —CH₂S(O)₂R¹⁵. In embodiments of the formulae above, R¹ⁱ is —CH₂S(O)₂N(R¹²)(R¹³).

The individual embodiments herein below, or combinations thereof, (e.g., embodiments of R^{1a}, R^{1b}, R^{1c}, R^{1d}, R^{1f}, R^{1g} and R^{1h}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ. In embodiments of the formulae above, two Ria bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ. In embodiments of the formulae above, R^{1h} and one of R^{1a}, R^{1b}, R¹⁰, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ. In embodiments of the formulae above, R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ.

The individual embodiments herein below, or combinations thereof, (e.g., embodiments of R⁹) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R¹⁹ is selected from:

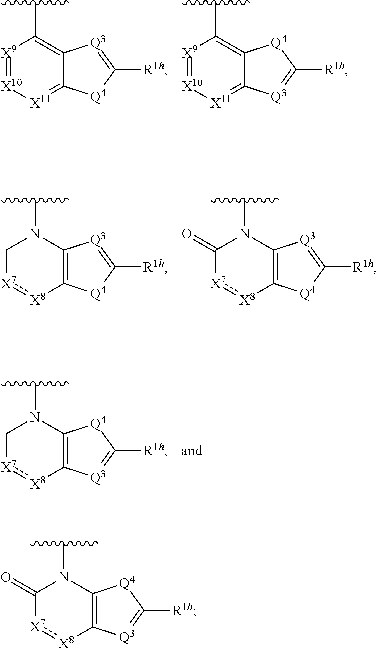

Q³ is independently N or C(R^{1d});
Q⁴ is independently O, S, C(R^{1a})(R^{1b}), or N(R^{1c});
X⁹, X¹⁰, and X¹¹ are independently selected from C(R^{1a}) or N;
X⁷ and X⁸ are independently selected from C(R^{1a}), C(R^{1a})(R^{1b}), N, or N(R^{1c});
each R^{1a}, R^{1b}, R^{1d}, R¹, R¹⁹, and R^{1h} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

In embodiments of the formulae above, R¹⁹ is selected from

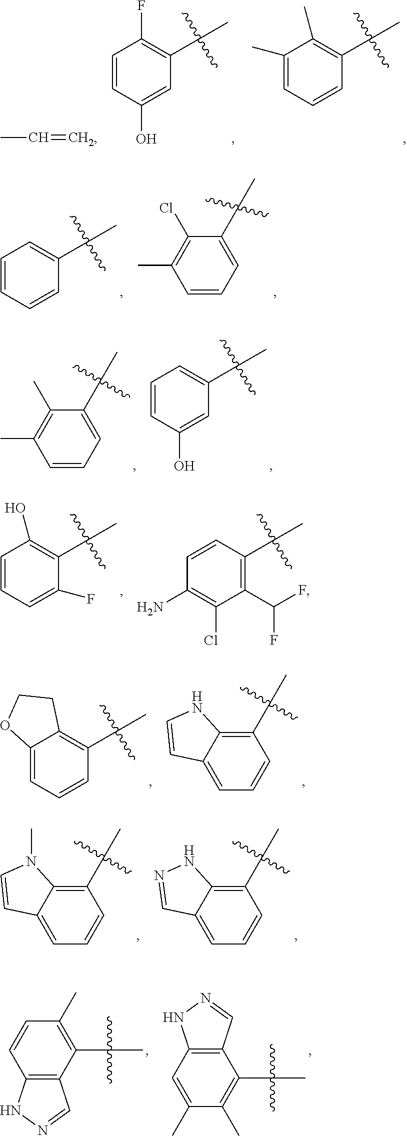

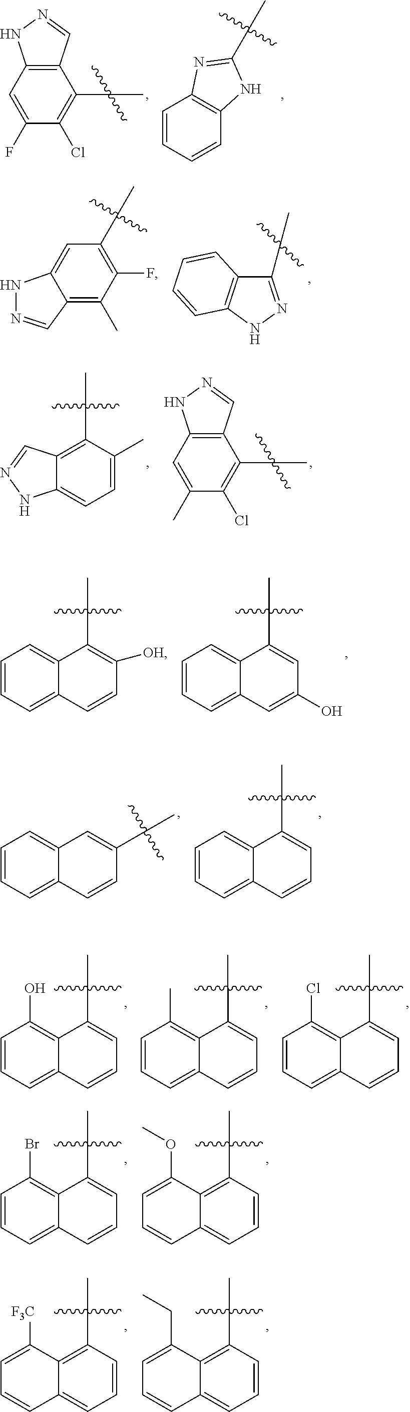

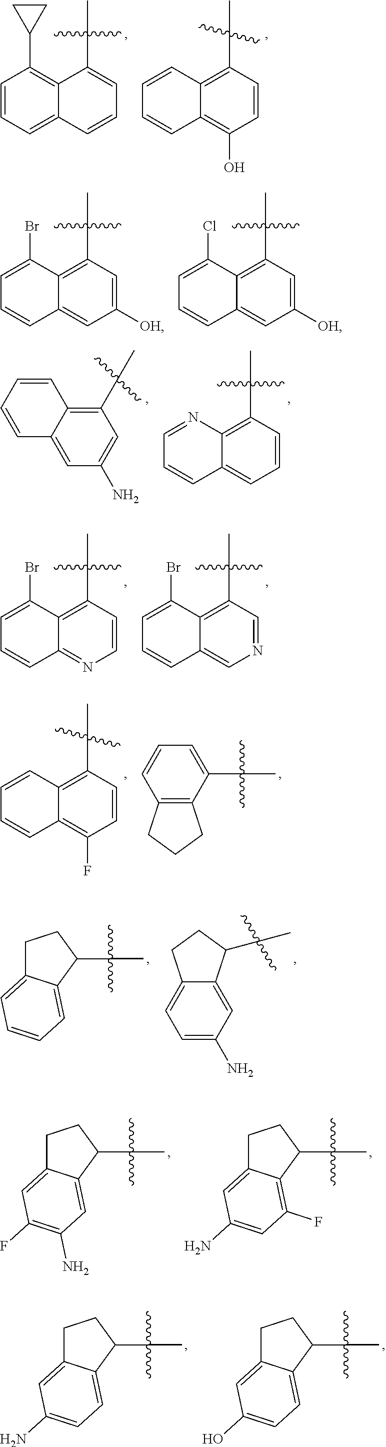

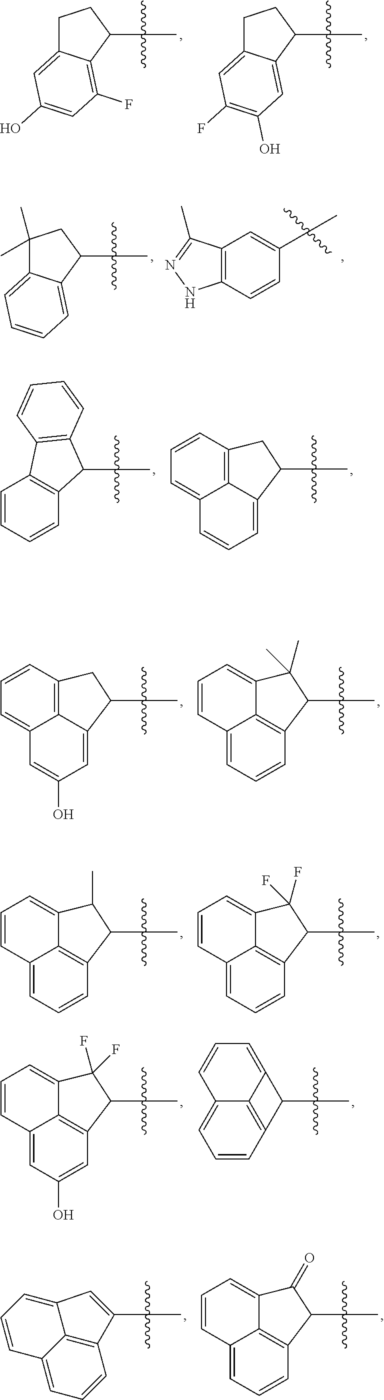

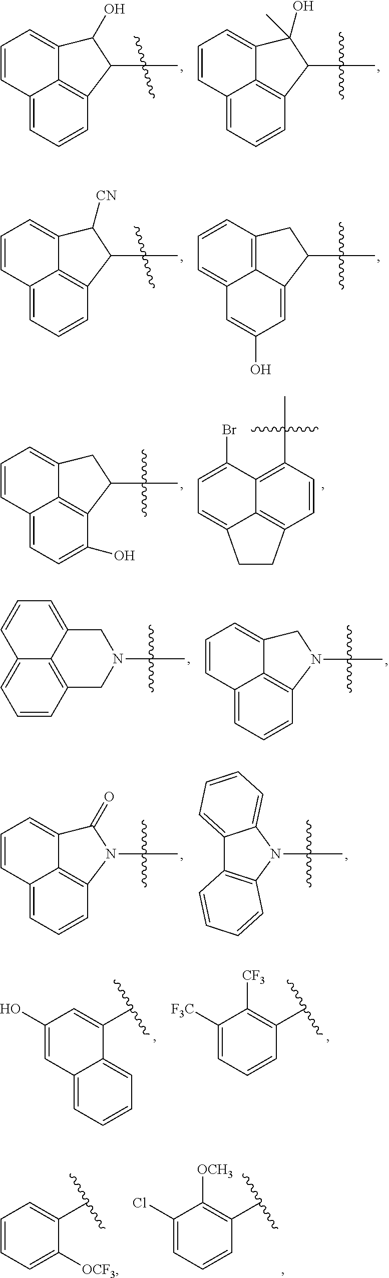

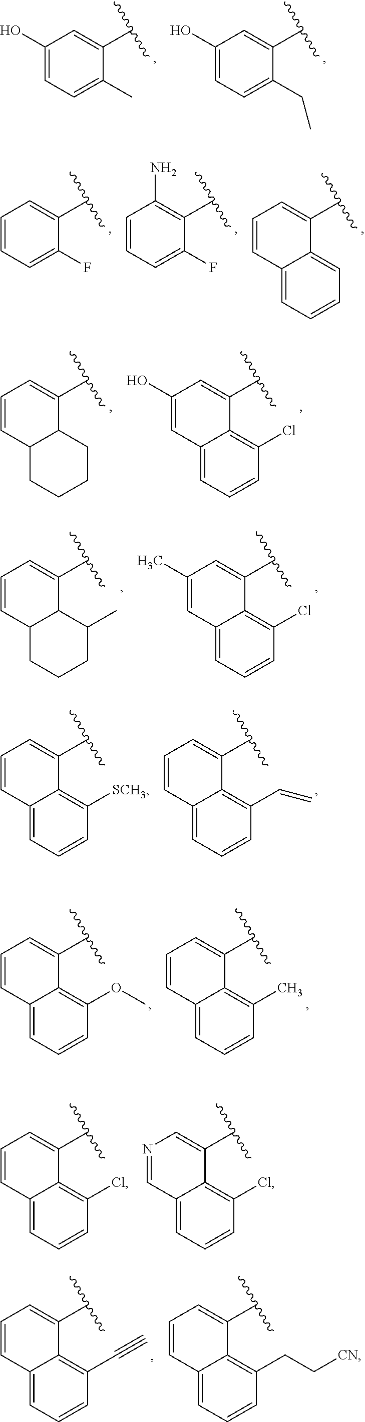

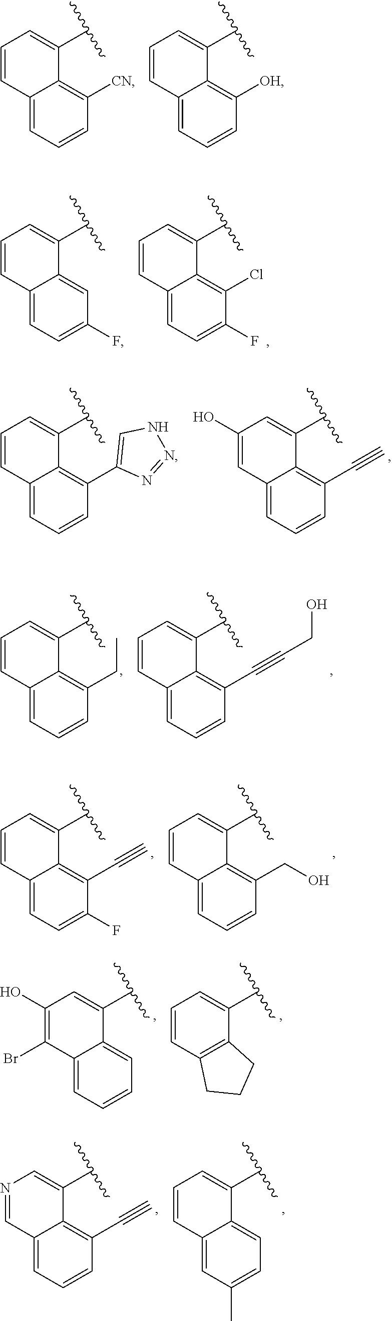

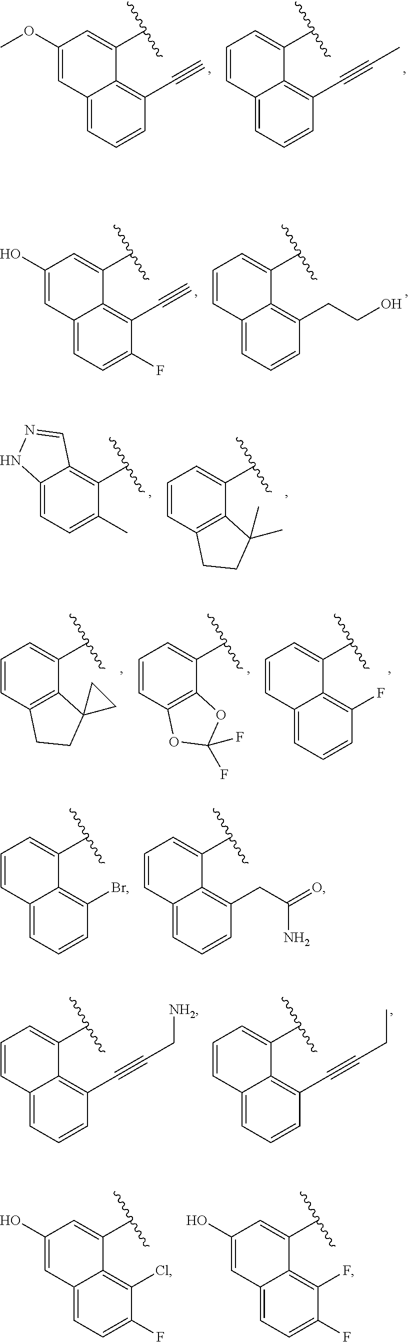

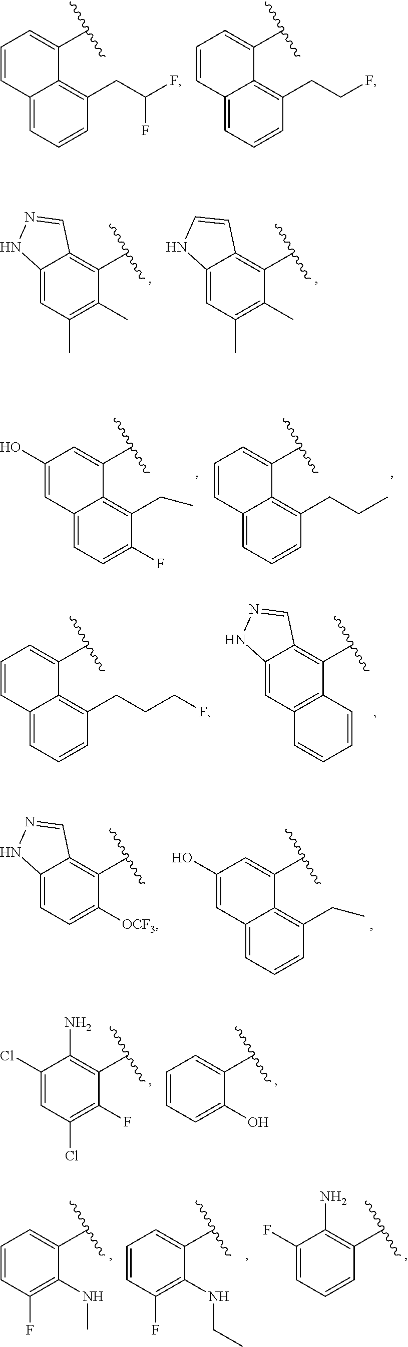

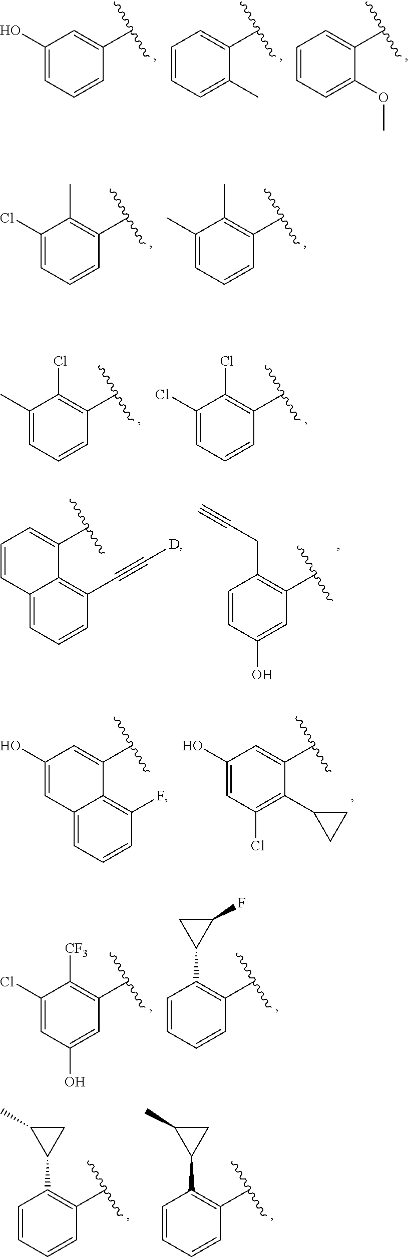

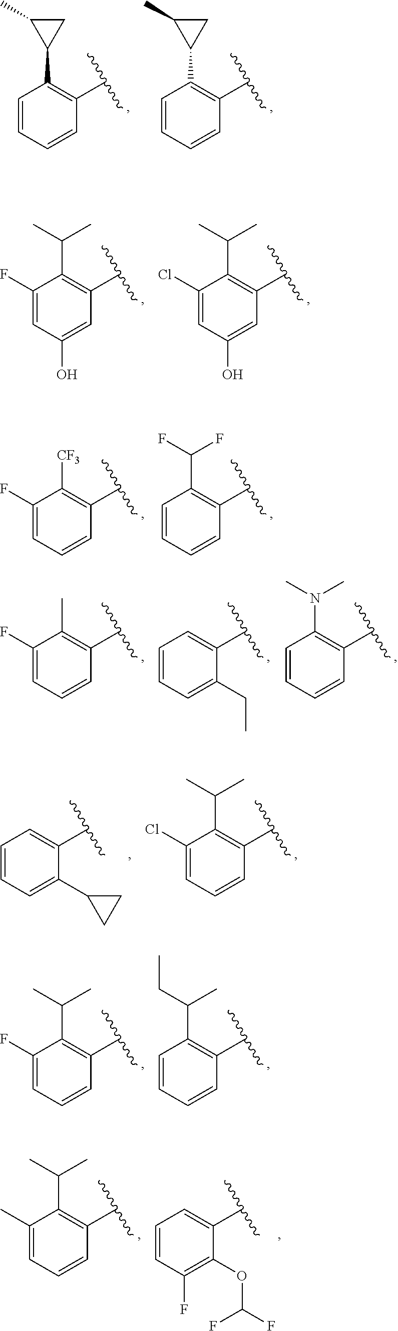

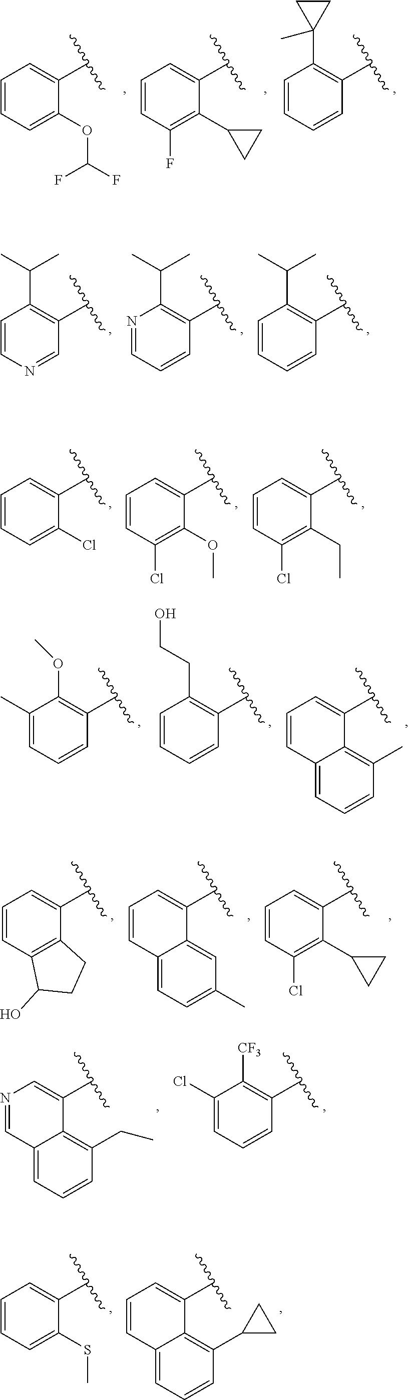

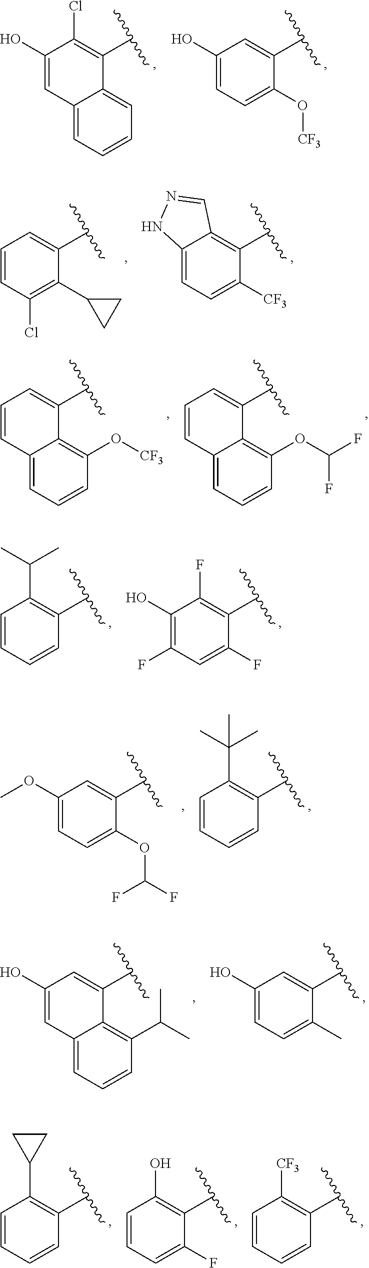

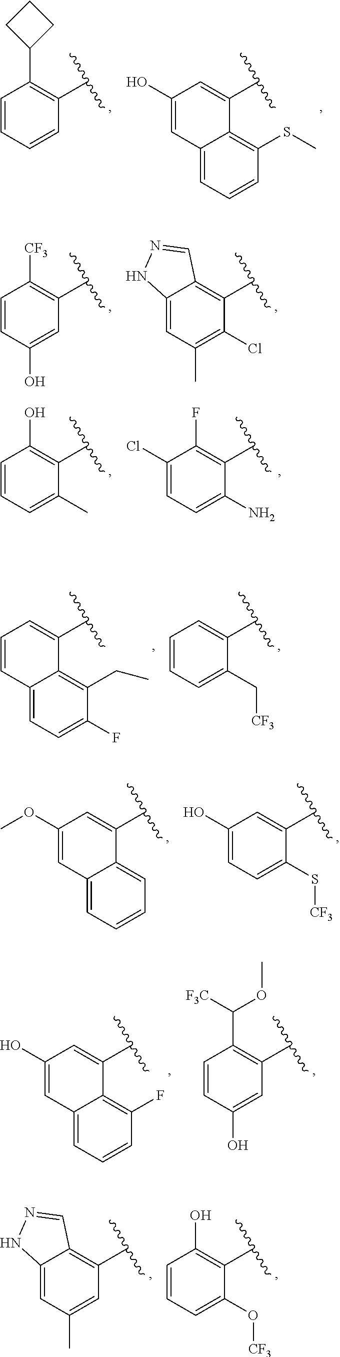

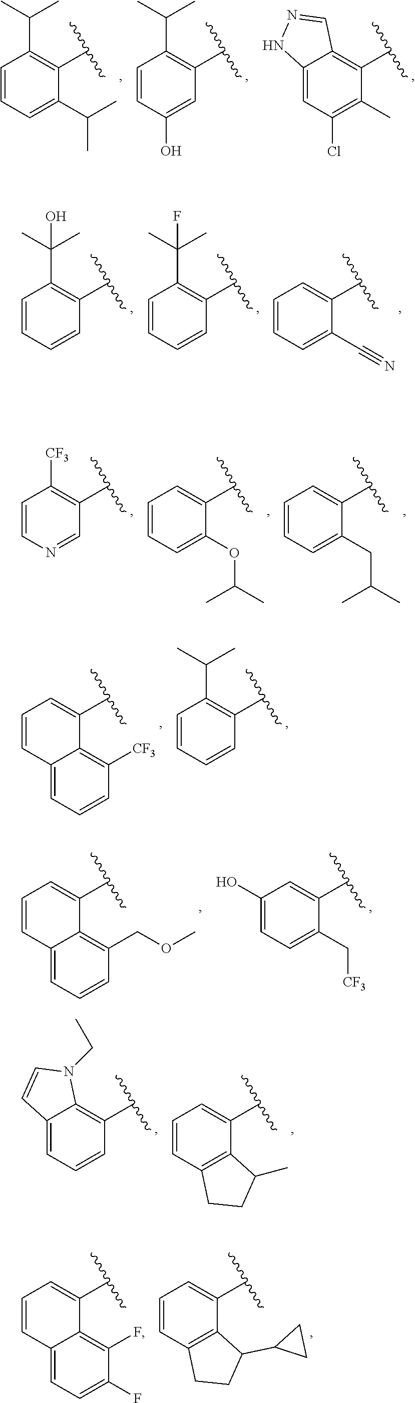

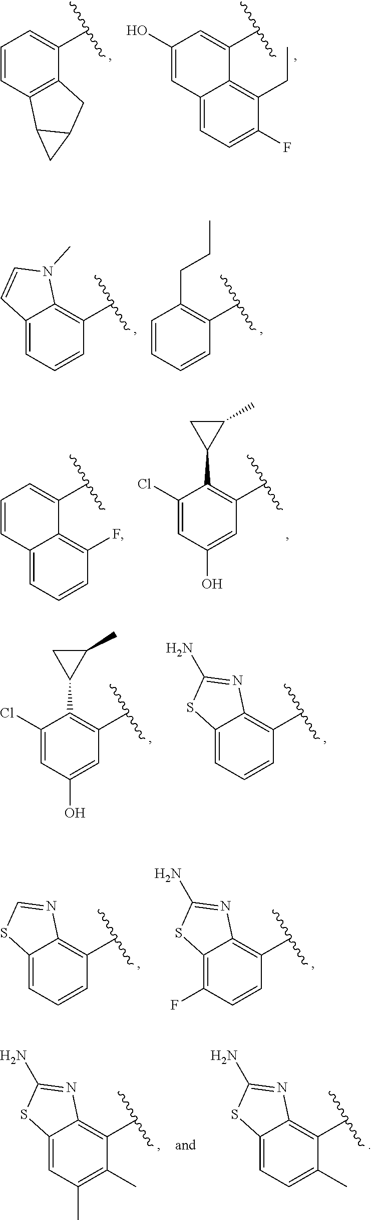

In embodiments of the formulae above, R¹⁹ is selected from

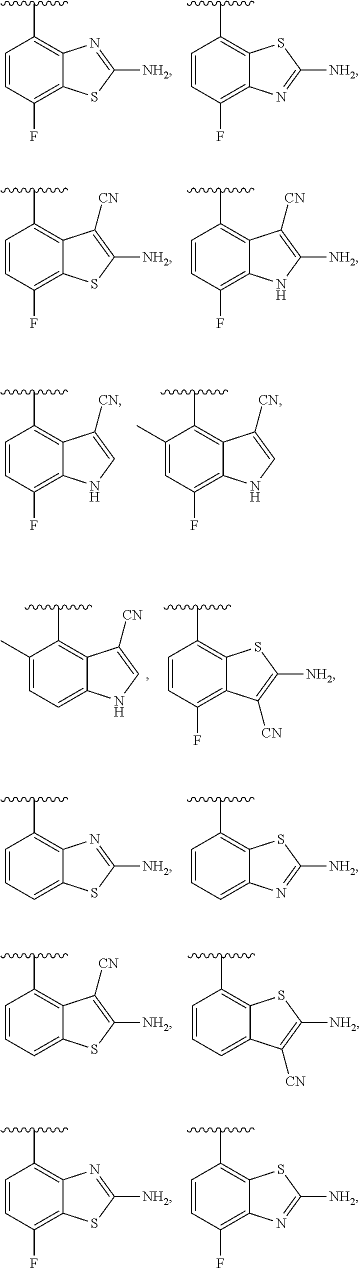

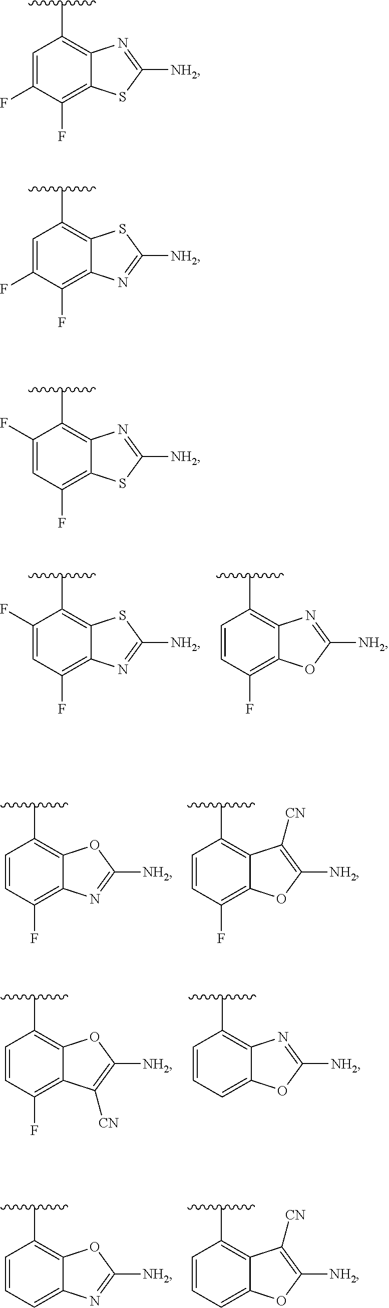

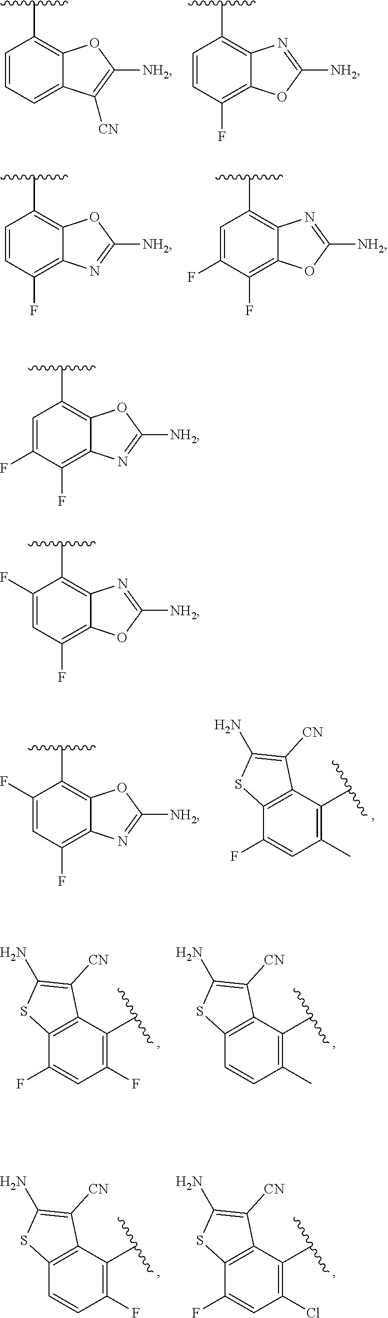

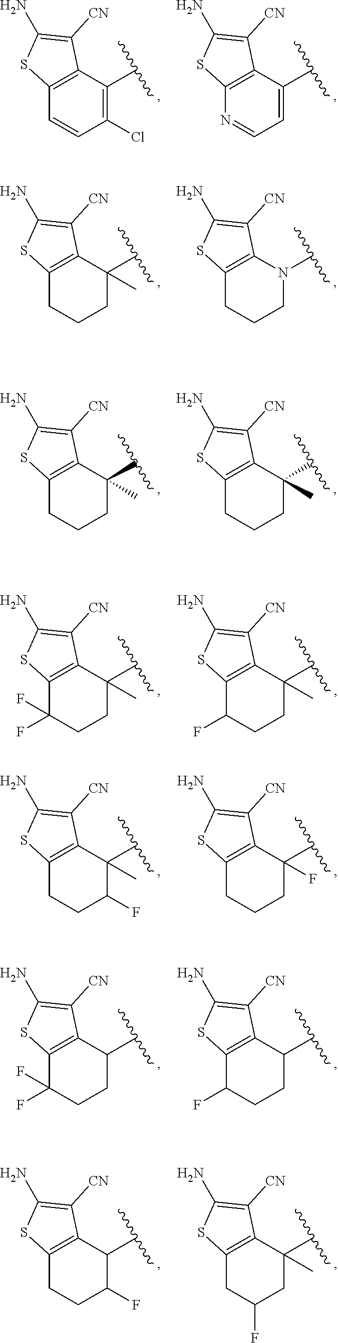

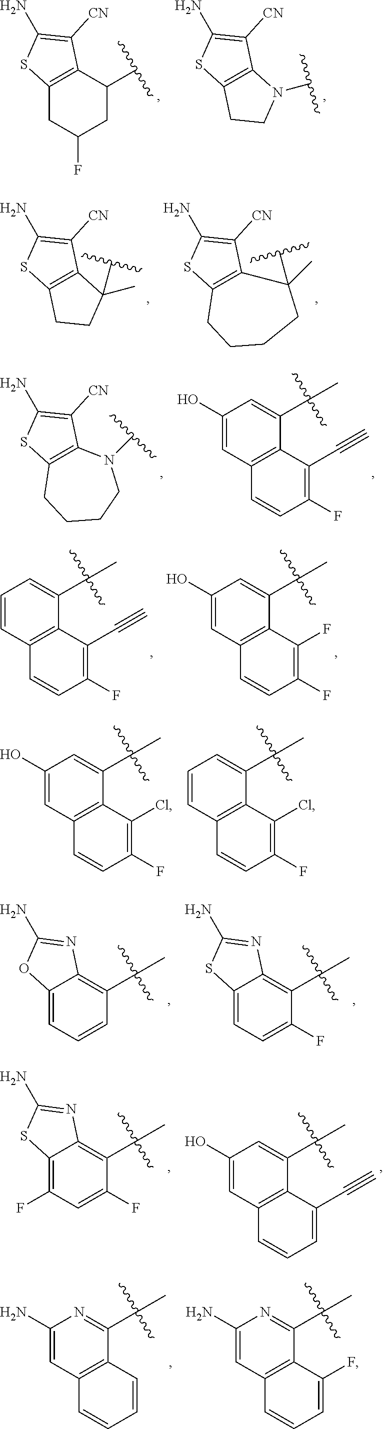

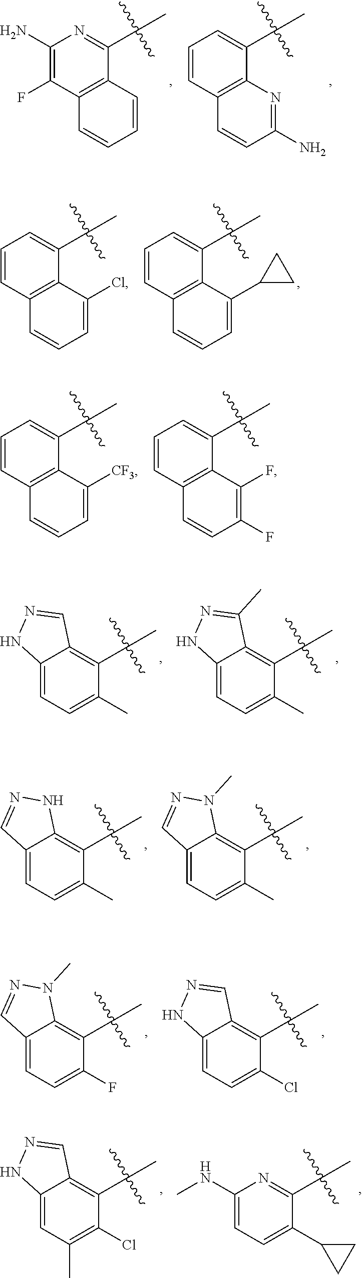

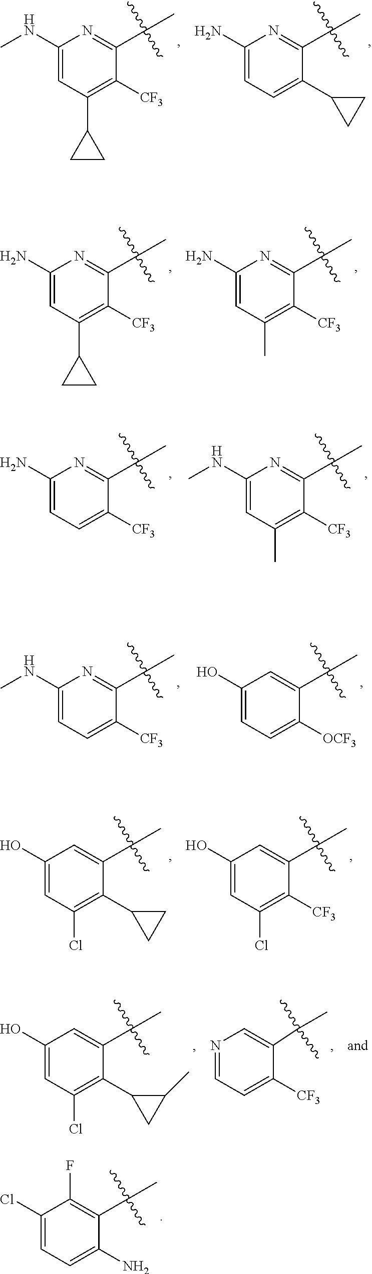

The individual embodiments herein below, or combinations thereof, (e.g., embodiments of R²) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R² is —OR^{12′}.

In embodiments of the formulae above, R² is selected from

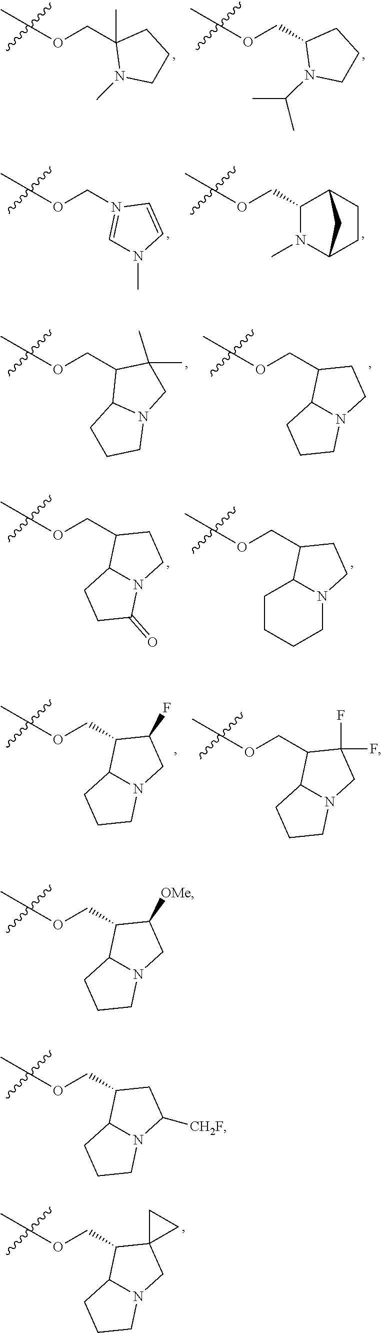

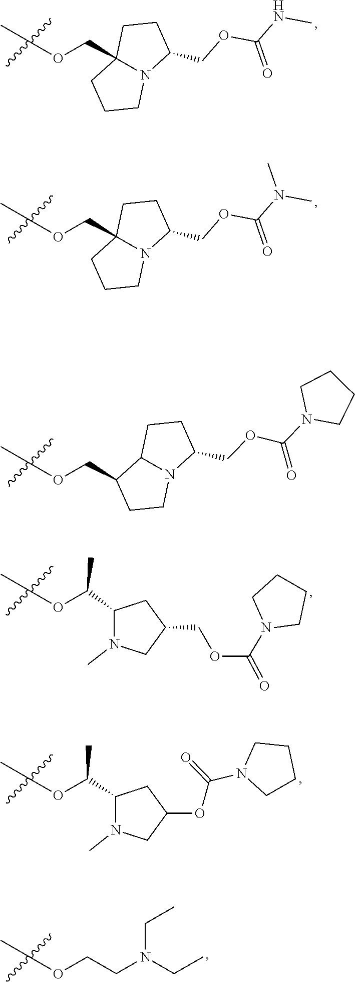

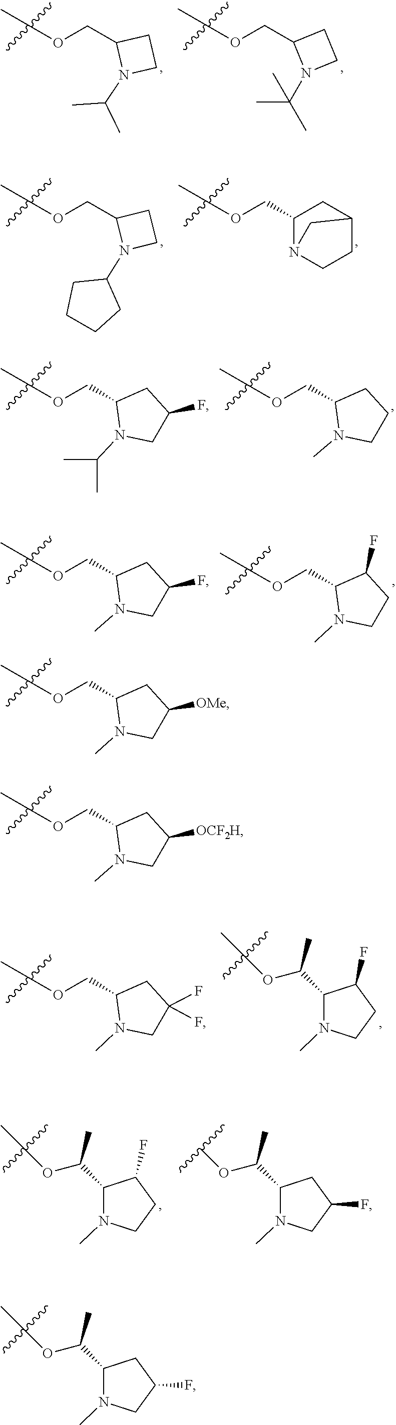

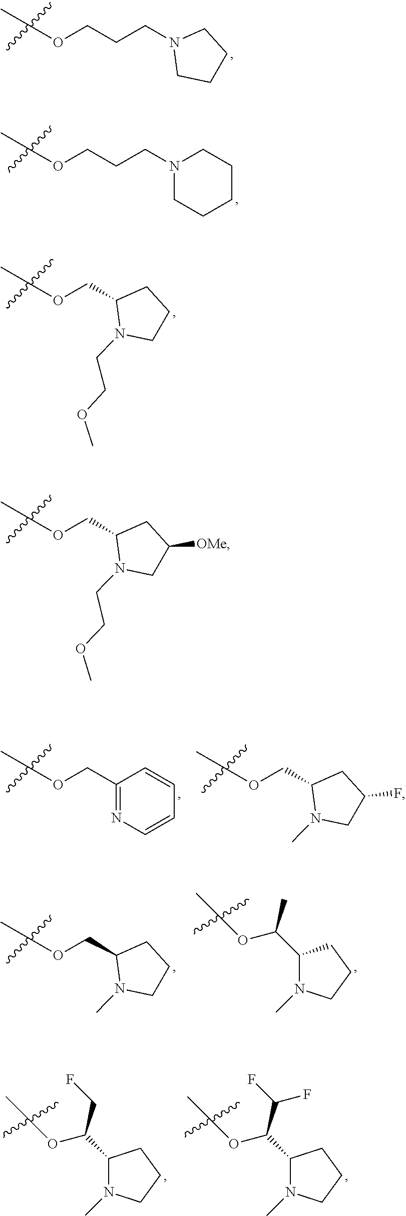

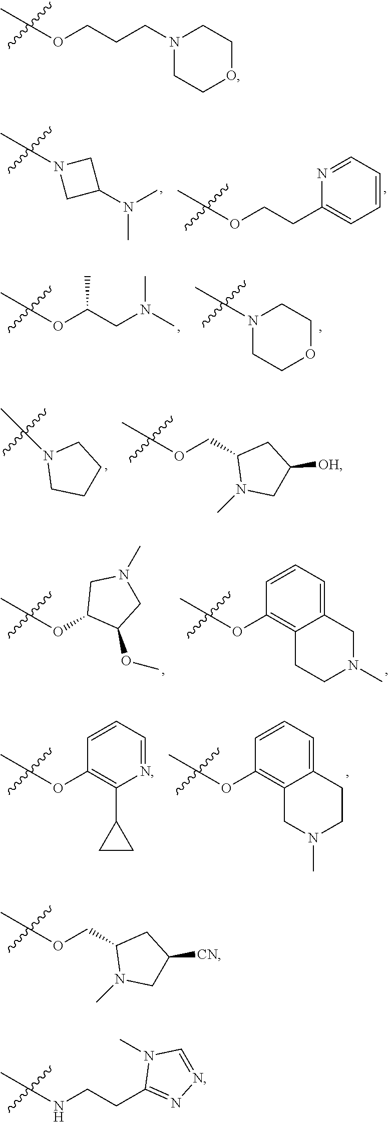

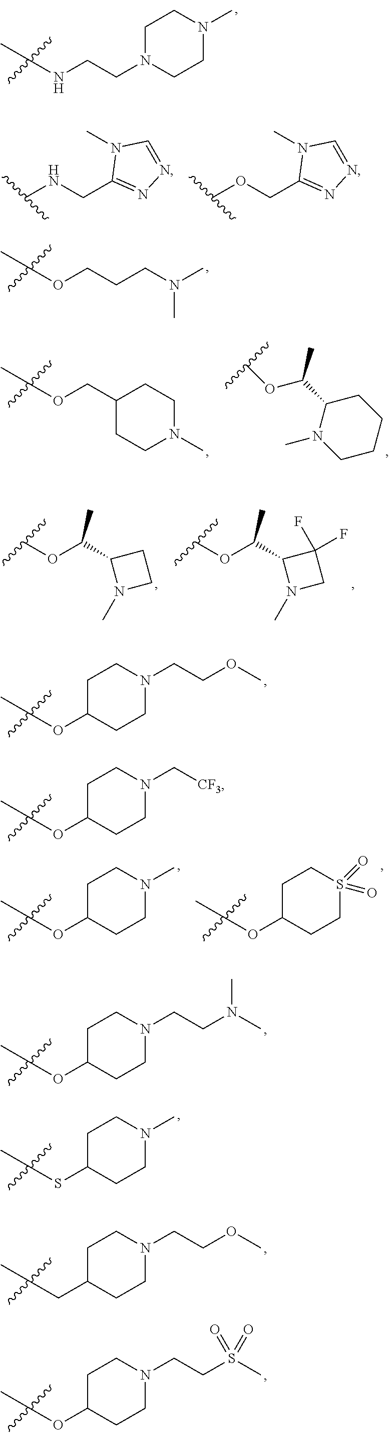

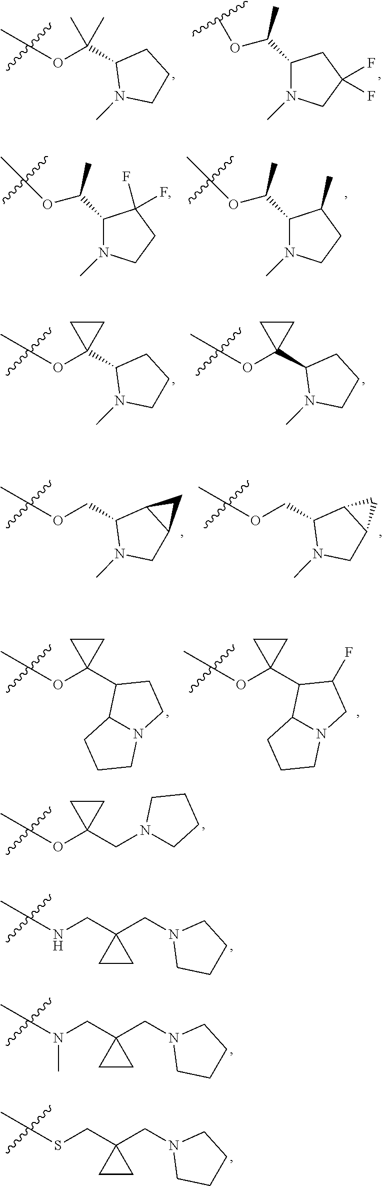

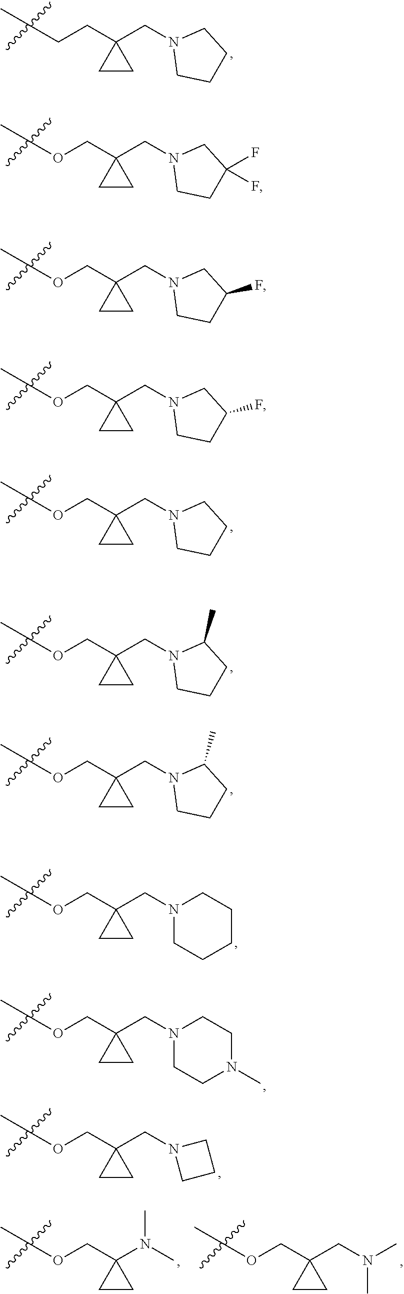

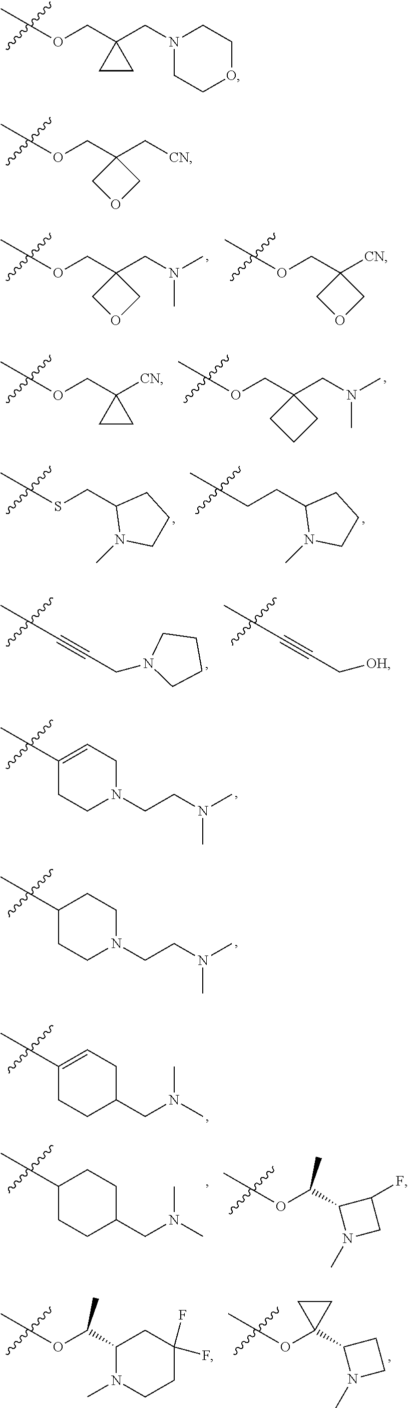

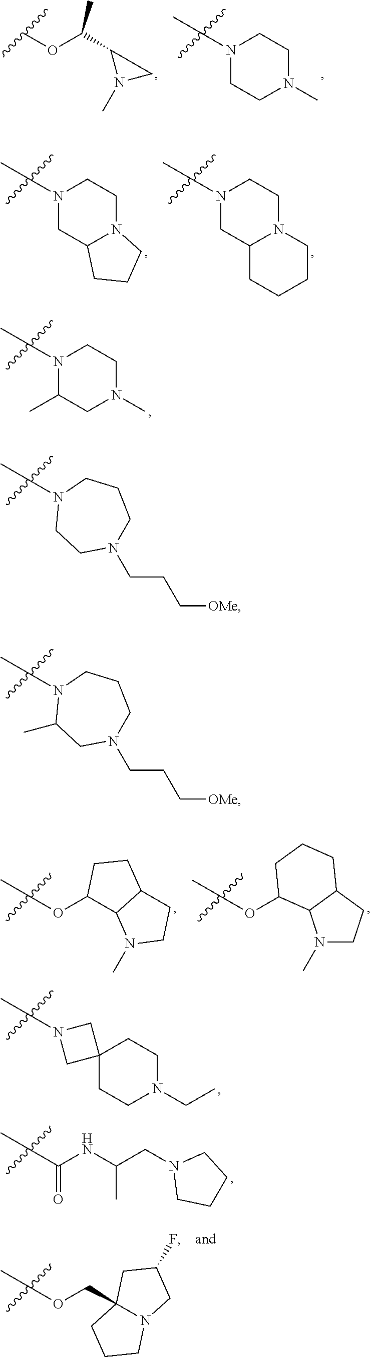

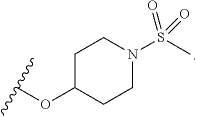 [SMILES: *OC1CCN(S(C)(=O)=O)CC1]

In embodiments of the formulae above, R² is selected from

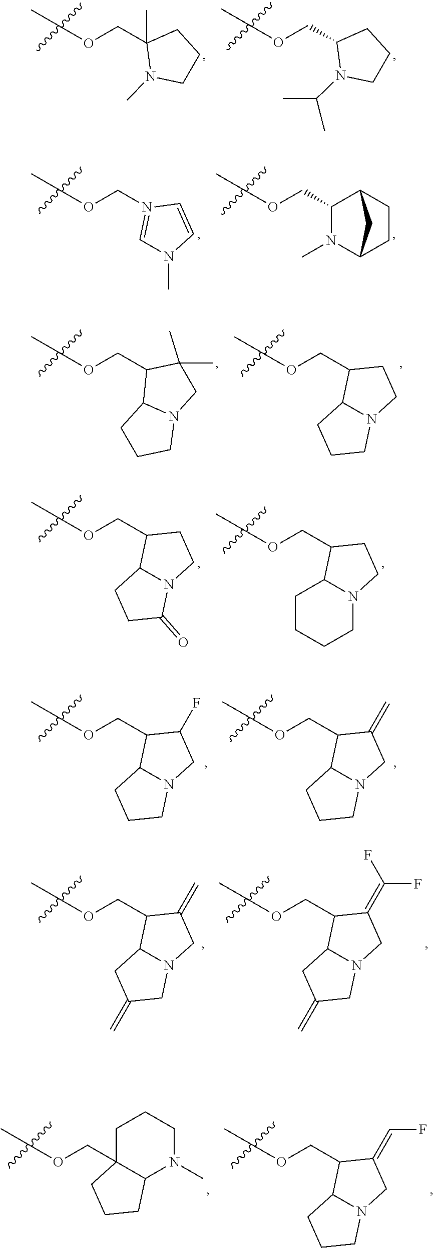

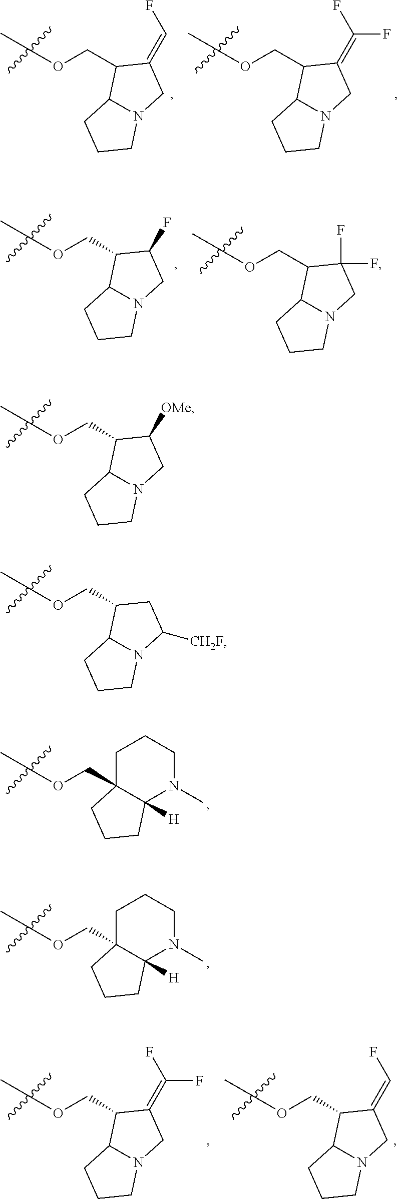

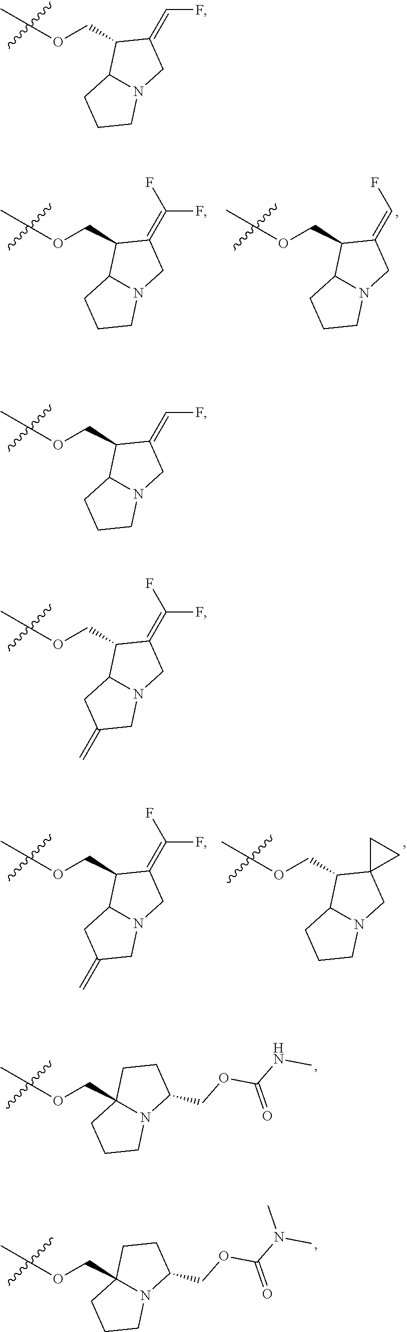

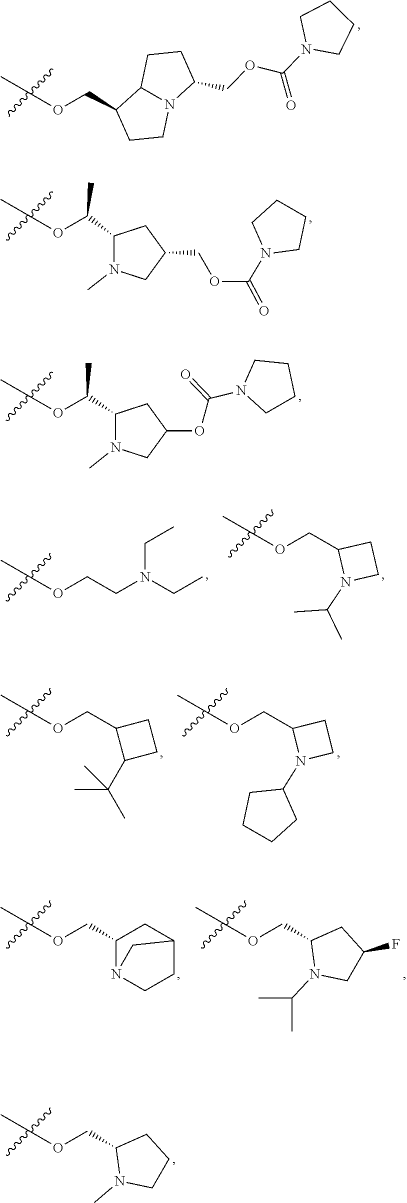

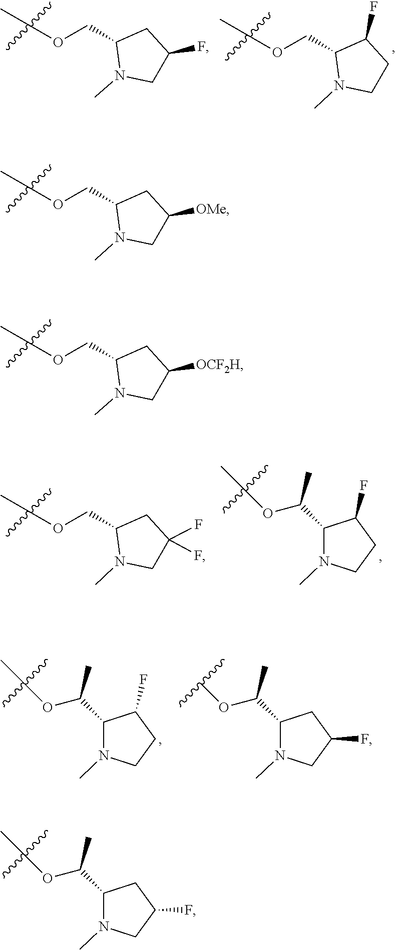

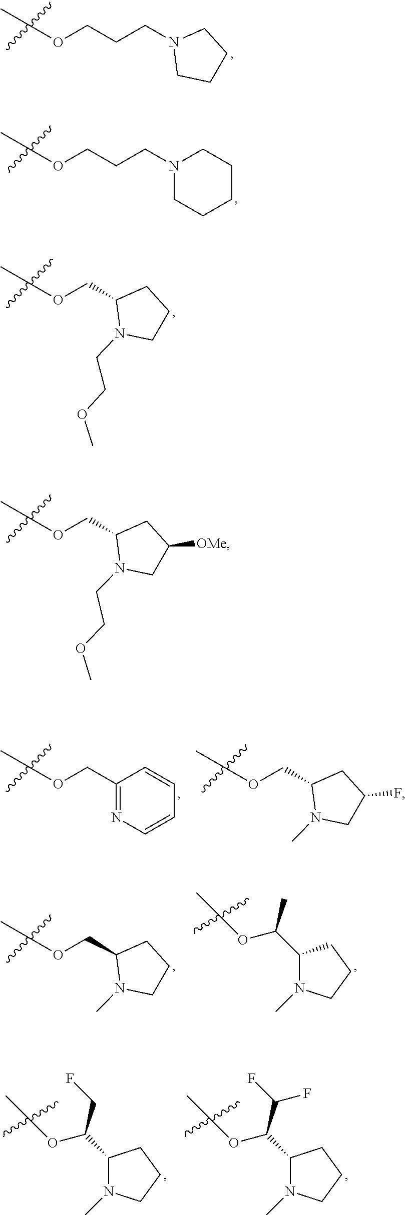

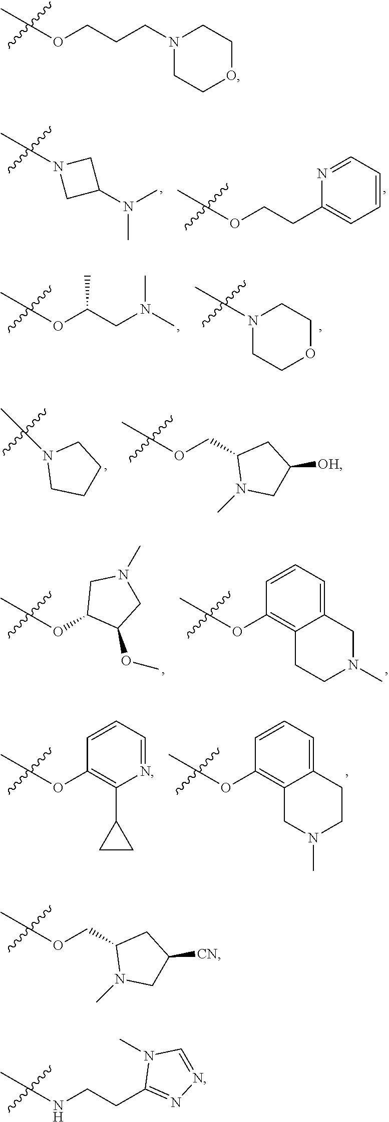

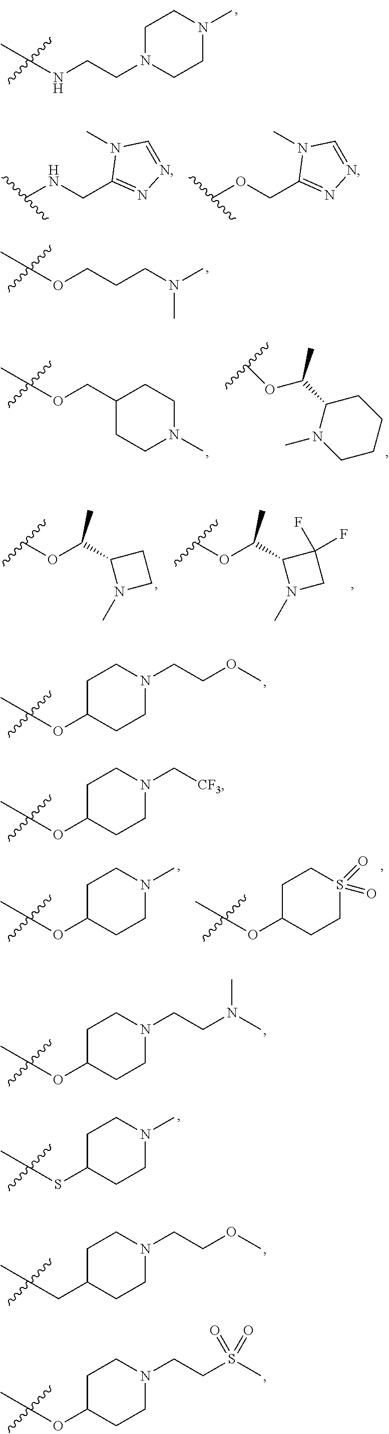

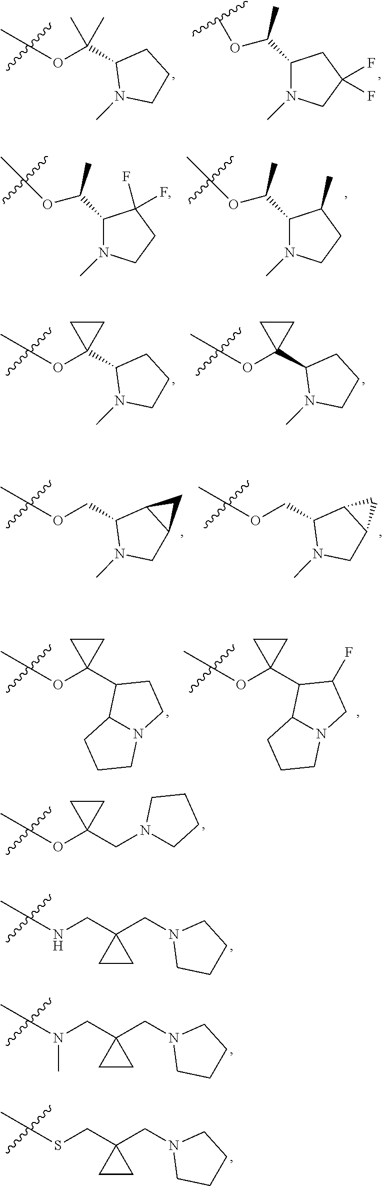

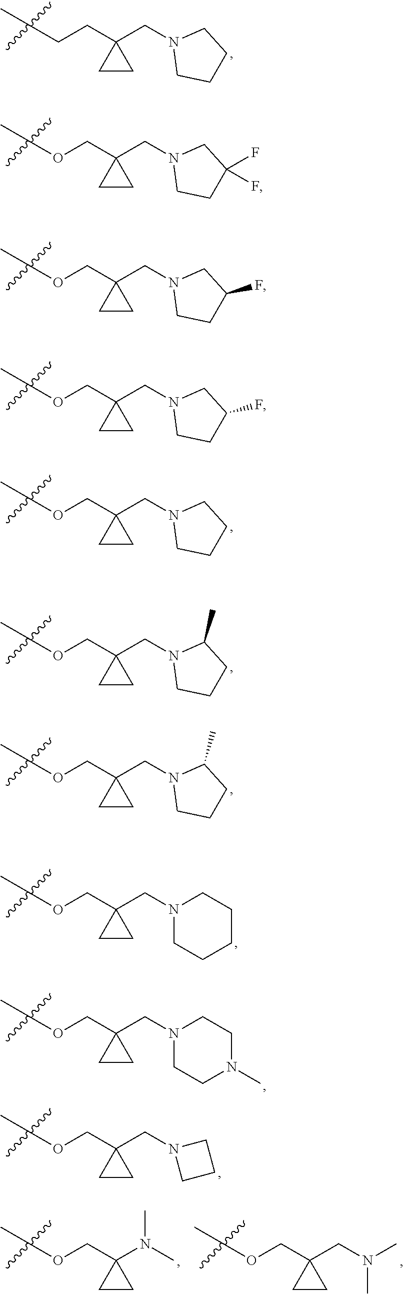

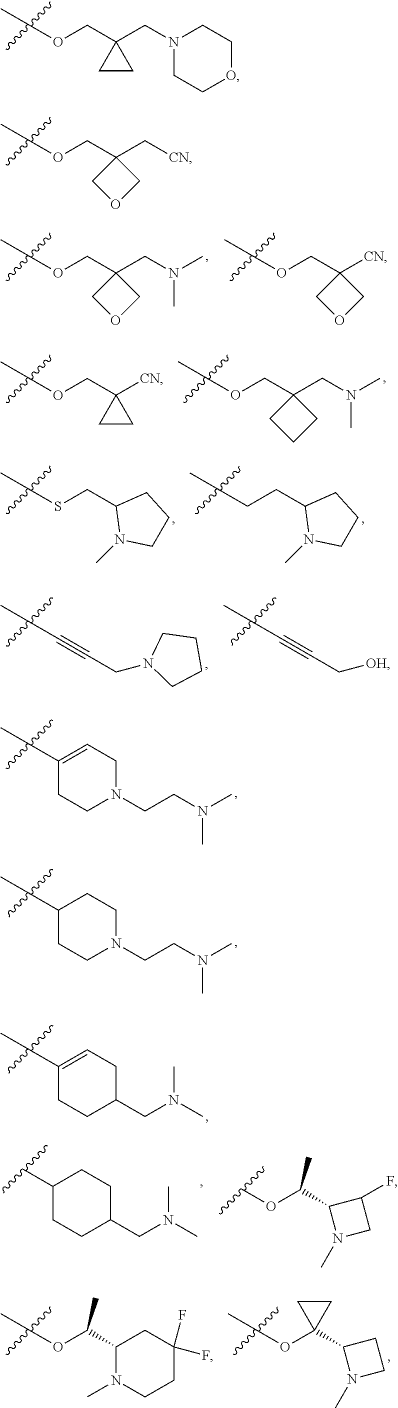

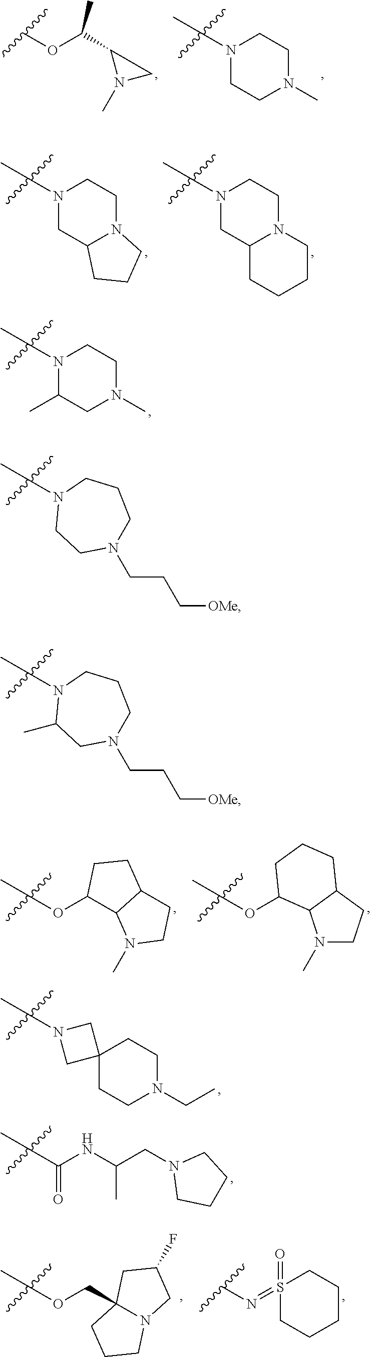

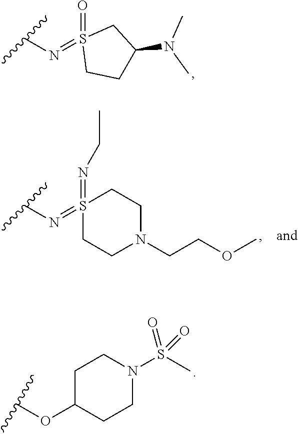

In embodiments of the formulae above, R^{2b} is selected from

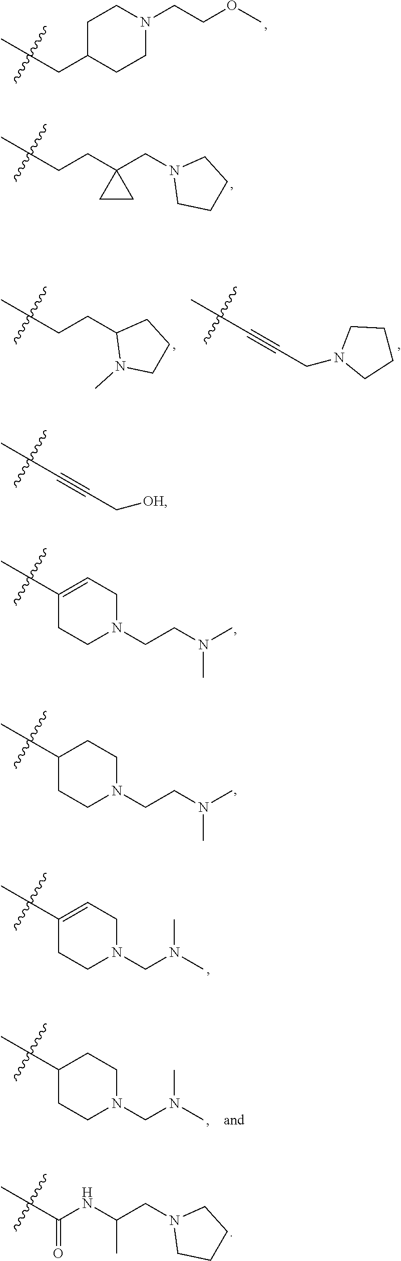

In embodiments of the formulae above, R² is

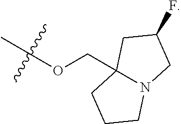 [SMILES: *OCC12CCCN1C[C@H](F)C2]

In embodiments of the formulae above, R² is

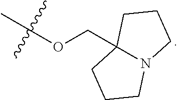 [SMILES: *OCC12CCCN1CCC2]

In embodiments of the formulae above, R² is

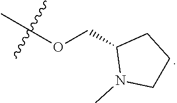 [SMILES: *OC[C@@H]1CCCN1C]

In embodiments of the formulae above, R² is

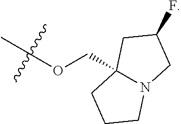 [SMILES: *OC[C@@]12CCCN1C[C@H](F)C2]

The individual embodiments herein below, or combinations thereof, (e.g., embodiments of L²) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, L² is a bond. In embodiments of the formulae above, L² is —O—. In embodiments of the formulae above, L² is —N(R^{4d})—. In embodiments of the formulae above, L² is —C(O)—. In embodiments of the formulae above, L² is —S—. In embodiments of the formulae above, L² is —S(O)₂—. In embodiments of the formulae above, L² is —S(O)—. In embodiments of the formulae above, L² is —P(O)R^{4d}—. In embodiments of the formulae above, L² is CR^{4c}R^{4c}. In embodiments of the formulae above, L² is —OCR^{4c}R^{4c}—. In embodiments of the formulae above, L² is —N(R^{4d})CR^{4c}R^{4c}—. In embodiments of the formulae above, L² is —C(O)CR^{4c}R^{4c}—. In embodiments of the formulae above, L² is —SCR^{4c}R^{4c}—. In embodiments of the formulae above, L² is —S(O)₂CR^{4c}R^{4c}—. In embodiments of the formulae above, L² is —S(O)CR^{4c}R^{4c}. In embodiments of the formulae above, L² is —P(O)R^{4d}CR^{4c}R^{4c}—. In embodiments of the formulae above, L² is —CR^{4c}R^{4c}CR^{4c}R^{4c}. In embodiments of the formulae above, L² is —CR^{4c}R^{4c}O—. In embodiments of the formulae above, L² is —CR^{4c}R^{4c}N(R^{4d})—. In embodiments of the formulae above, L² is —CR^{4c}R^{4c}C(O)—. In embodiments of the formulae above, L² is —CR^{4c}R^{4c}S—. In embodiments of the formulae above, L² is —CR^{4c}R^{4c}S(O)₂—. In embodiments of the formulae above, L² is —CR^{4c}R^{4c}S(O)—. In embodiments of the formulae above, L² is —CR^{4c}R^{4c}P(O)R^{4d}—. In embodiments of the formulae above, L² is —N(R^{4d})C(O)—. In embodiments of the formulae above, L² is —N(R^{4d})S(O)₂—. In embodiments of the formulae above, L² is —N(R^{4d})S(O)—. In embodiments of the formulae above, L² is —N(R^{4d})P(O)R^{4d}—. In embodiments of the formulae above, L² is —C(O)N(R^{4d})—. In embodiments of the formulae above, L² is —S(O)₂N(R^{4d})—. In embodiments of the formulae above, L² is —S(O)N(R^{4d})—. In embodiments of the formulae above, L² is —P(O)R^{4d}N(R^{4d})—. In embodiments of the formulae above, L² is —OC(O)—. In embodiments of the formulae above, L² is —OS(O)₂—. In embodiments of the formulae above, L² is —OS(O)—. In embodiments of the formulae above, L² is —OP(O)R^{4d}—. In embodiments of the formulae above, L² is —C(O)O—. In embodiments of the formulae above, L² is —S(O)₂O—. In embodiments of the formulae above, L² is —S(O)O—. In embodiments of the formulae above, L² is —P(O)R^{4d}O—. In embodiments of the formulae above, L² is —CR^{4c}R^{4c}CR^{4c}R^{4c}CR^{4c}R^{4c}—. In embodiments of the formulae above, L² is —OCR^{4c}R^{4c}R^{4c}R^{4c}—. In embodiments of the formulae above, L² is —N(R^{4d})CR^{4c}R^{4c}CR^{4c}R^{4c}—. In embodiments of the formulae above, L² is —C(O)CR^{4c}R^{4c}R^{4c}R^{4c}—. In embodiments of the formulae above, L² is —SCR^{4c}R^{4c}CR^{4c}R^{4c}—. In embodiments of the formulae above, L² is —S(O)₂CR^{4c}R^{4c}R^{4c}R^{4c}—. In embodiments of the formulae above, L² is —S(O)CR^{4c}R^{4c}CR^{4c}R^{4c}—. In embodiments of the formulae above, L² is —P(O)R^{4d}CR^{4c}R^{4c}CR^{4c}R^{4c}—. In embodiments of the formulae above, L² is —CR^{4c}R^{4c}R^{4c}R^{4c}O—. In embodiments of the formulae above, L² is —CR^{1c}R^{4c}R^{4c}R^{4c}N(R^{4d})—. In embodiments of the formulae above, L² is —CR^{4c}R^{4c}CR^{4c}R^{4c}C(O)—. In embodiments of the formulae above, L² is —CR^{4c}R^{4c}CR^{4c}R^{4c}S—. In embodiments of the formulae above, L² is —CR^{4c}R^{4c}CR^{4c}R^{4c}S(O)₂—. In embodiments of the formulae above, L² is —CR^{4c}R^{4c}CR^{4c}R^{4c}S(O)—. In embodiments of the formulae above, L² is —CR^{4c}R^{4c}CR^{4c}R^{4c}P(O)R^{4d}—. In embodiments of the formulae above, L² is —CR^{1c}R^{4c}R^{4c}R^{4c}N(R^{4d})C(O)—. In embodiments of the formulae above, L² is —CR^{4c}R^{4c}N(R^{4d})C(O)—. In embodiments of the formulae above, L² is —CR^{4c}R^{4c}CR^{4c}R^{4c}C(O)N(R^{4d})—. In embodiments of the formulae above, L² is —CR^{4c}R^{4c}C(O)N(R^{4d})—.

In embodiments of the formulae above, L² is —NH—. In embodiments of the formulae above, L² is —P(O)CH₃—. In embodiments of the formulae above, L² is CH₂. In embodiments of the formulae above, L² is —OCH₂—. In embodiments of the formulae above, L² is —N(H)CH₂—. In embodiments of the formulae above, L² is —C(O)CH₂—. In embodiments of the formulae above, L² is —SCH₂—. In embodiments of the formulae above, L² is —S(O)₂CH₂—. In embodiments of the formulae above, L² is —S(O)CH₂—. In embodiments of the formulae above, L² is —P(O)(CH₃)CH₂—. In embodiments of the formulae above, L² is —CH₂CH₂—. In embodiments of the formulae above, L² is —CH₂O—. In embodiments of the formulae above, L² is —CH₂N(H)—. In embodiments of the formulae above, L² is —CH₂C(O)—. In embodiments of the formulae above, L² is —CH₂S—. In embodiments of the formulae above, L² is —CH₂S(O)₂—. In embodiments of the formulae above, L² is —CH₂S(O)—. In embodiments of the formulae above, L² is —CH₂P(O)CH₃—. In embodiments of the formulae above, L² is —N(H)C(O)—. In embodiments of the formulae above, L² is —N(H)S(O)₂—. In embodiments of the formulae above, L² is —N(H)S(O)—. In embodiments of the formulae above, L² is —N(H)P(O)CH₃—. In embodiments of the formulae above, L² is —C(O)N(H)—. In embodiments of the formulae above, L² is —S(O)₂N(H)—. In embodiments of the formulae above, L² is —S(O)N(H)—. In embodiments of the formulae above, L² is —P(O)(CH₃)N(H)—. In embodiments of the formulae above, L² is —OC(O)—. In embodiments of the formulae above, L² is —OS(O)₂—. In embodiments of the formulae above, L² is —OS(O)—. In embodiments of the formulae above, L² is —OP(O)CH₃—. In embodiments of the formulae above, L² is —C(O)O—. In embodiments of the formulae above, L² is —S(O)₂O—. In embodiments of the formulae above, L² is —S(O)O—. In embodiments of the formulae above, L² is —P(O)(CH₃)O—. In embodiments of the formulae above, L² is —CH₂CH₂CH₂—. In embodiments of the formulae above, L² is —OCH₂CH₂—. In embodiments of the formulae above, L² is —N(H)CH₂CH₂—. In embodiments of the formulae above, L² is —C(O)CH₂CH₂—. In embodiments of the formulae above, L² is —SCH₂CH₂—. In embodiments of the formulae above, L² is —S(O)₂CH₂CH₂—. In embodiments of the formulae above, L² is —S(O)CH₂CH₂—. In embodiments of the formulae above, L² is —P(O)(CH₃)CH₂CH₂—. In embodiments of the formulae above, L² is —CH₂CH₂O—. In embodiments of the formulae above, L² is —CH₂CH₂N(H)—. In embodiments of the formulae above, L² is —CH₂CH₂C(O)—. In embodiments of the formulae above, L² is —CH₂CH₂S—. In embodiments of the formulae above, L² is —CH₂CH₂S(O)₂—. In embodiments of the formulae above, L² is —CH₂CH₂S(O)—. In embodiments of the formulae above, L² is —CH₂CH₂P(O)(CH₃)—. In embodiments of the formulae above, L² is —CH₂CH₂N(H)C(O)—. In embodiments of the formulae above, L² is —CH₂N(H)C(O)—. In embodiments of the formulae above, L² is —CH₂CH₂C(O)N(H)—. In embodiments of the formulae above, L² is —CH₂C(O)N(H)—.

The individual embodiments herein below, or combinations thereof, (e.g., embodiments of R^{4c}) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, R^{4c} is independently hydrogen. In embodiments of the formulae above, R^{4c} is independently halogen. In embodiments of the formulae above, R^{4c} is independently —CN. In embodiments of the formulae above, R^{4c} is independently C₁₋₆alkyl. In embodiments of the formulae above, R⁴ is independently C₂₋₆alkenyl. In embodiments of the formulae above, R^{4c} is independently C₂₋₆alkynyl. In embodiments of the formulae above, R⁴ is independently C₁₋₆haloalkyl. In embodiments of the formulae above, R^{4c} is independently C₁₋₆alkoxy. In embodiments of the formulae above, R⁴ is independently C₁₋₆haloalkoxy. In embodiments of the formulae above, R^{4c} is independently C₃₋₁₀cycloalkyl. In embodiments of the formulae above, R^{4c} is independently —CH₂—C₃₋₁₀cycloalkyl. In embodiments of the formulae above, R^{4c} is independently C₂₋₉heterocycloalkyl. In embodiments of the formulae above, R^{4c} is independently —CH₂—C₂₋₉heterocycloalkyl. In embodiments of the formulae above, R^{4c} is independently —OR¹⁴. In embodiments of the formulae above, R^{4c} is independently —SR¹⁴. In embodiments of the formulae above, R⁴ is independently —C(O)OR¹⁴. In embodiments of the formulae above, R⁴ is independently —C(O)N(R¹⁴)(R¹⁴). In embodiments of the formulae above, R^{4c} is independently —C(O)C(O)N(R¹⁴)(R¹⁴). In embodiments of the formulae above, R⁴ is independently —OC(O)N(R¹⁴)(R¹⁴). In embodiments of the formulae above, R⁴ is independently —C(O)R¹⁴. In embodiments of the formulae above, R^{4c} is independently —S(O)₂R¹⁴. In embodiments of the formulae above, R^{4c} is independently —S(O)₂N(R¹⁴)(R¹⁴). In embodiments of the formulae above, R^{4c} is independently —OCH₂C(O)OR¹⁴. In embodiments of the formulae above, R^{4c} is independently —OC(O)R^{14a}. In embodiments of the formulae above, R^{4c} is independently —N(R¹⁴)(R¹⁴). In embodiments of the formulae above, R^{4c} is independently —N(R¹⁴)C(O)N(R¹⁴)(R¹⁴). In embodiments of the formulae above, R^{4c} is independently —N(R¹⁴)C(O)OR¹⁴. In embodiments of the formulae above, R^{4c} is independently —N(R¹⁴)C(O)R¹⁴. In embodiments of the formulae above, R⁴ is independently —N(R¹⁴)S(O)₂R¹⁴.

In embodiments of the formulae above, R⁴ is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4c} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R¹⁴, —S(O)₂R¹⁴, —S(O)₂N(R¹⁴)(R¹⁴), and —OC(O)R¹⁴. In embodiments of the formulae above, R^{4c} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R¹⁴, —S(O)₂R¹⁴, —S(O)₂N(R¹⁴)(R¹⁴), and —OC(O)R^{14a}. In embodiments of the formulae above, R^{4c} is independently C₁₋₆haloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R⁴ is independently C₁₋₆alkoxy optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4c} is independently C₁₋₆haloalkoxy optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R⁴ is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R¹ is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4c} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴)₉, —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R⁴ is independently —CH₂—C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}.

The individual embodiments herein below, or combinations thereof, (e.g., embodiments of R^{4d}) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, R^{4d} is independently hydrogen. In embodiments of the formulae above, R^{4d} is independently —CN. In embodiments of the formulae above, R^{4d} is independently C₁₋₆alkyl. In embodiments of the formulae above, R^{4d} is independently C₂₋₆alkenyl. In embodiments of the formulae above, R^{4d} is independently C₂₋₆alkynyl. In embodiments of the formulae above, R^{4d} is independently C₁₋₆haloalkyl. In embodiments of the formulae above, R^{4d} is independently C₁₋₆alkoxy. In embodiments of the formulae above, R^{4d} is independently C₁₋₆ haloalkoxy. In embodiments of the formulae above, R^{4d} is independently C₃₋₁₀cycloalkyl. In embodiments of the formulae above, R^{4d} is independently —CH₂—C₃₋₁₀cycloalkyl. In embodiments of the formulae above, R^{4d} is independently C₂₋₉heterocycloalkyl. In embodiments of the formulae above, R^{4d} is independently —CH₂—C₂₋₉heterocycloalkyl. In embodiments of the formulae above, R^{4d} is independently —OR¹⁴. In embodiments of the formulae above, R^{4d} is independently —SR¹⁴. In embodiments of the formulae above, R^{4d} is independently —C(O)OR¹⁴. In embodiments of the formulae above, R^{4d} is independently —C(O)N(R¹⁴)(R¹⁴). In embodiments of the formulae above, R^{4d} is independently —C(O)C(O)N(R¹⁴)(R¹⁴). In embodiments of the formulae above, R^{4d} is independently —OC(O)N(R¹⁴)(R¹⁴). In embodiments of the formulae above, R^{4d} is independently —C(O)R^{14a}. In embodiments of the formulae above, R^{4d} is independently —S(O)₂R¹⁴. In embodiments of the formulae above, R^{4d} is independently —S(O)₂N(R¹⁴)(R¹⁴). In embodiments of the formulae above, R^{4d} is independently —OCH₂C(O)OR¹⁴. In embodiments of the formulae above, R^{4d} is independently —OC(O)R^{14a}.

In embodiments of the formulae above, R^{4d} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4d} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4d} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴)₉, —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{4a}, —S(O)₂R¹⁴, —S(O)₂N(R¹⁴)(R¹⁴), and —OC(O)R^{14a}. In embodiments of the formulae above, R¹ is independently C₁₋₆haloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4d} is independently C₁₋₆alkoxy optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R¹⁴. In embodiments of the formulae above, R^{4d} is independently C₁₋₆haloalkoxy optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4d} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4d} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}. In embodiments of the formulae above, R^{4d} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{4a}, —S(O)₂R¹⁴, —S(O)₂N(R¹⁴)(R¹⁴), and —OC(O)R^{14a}. In embodiments of the formulae above, R^{4d} is independently —CH₂—C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴)₉, —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a}.

The individual embodiments herein below, or combinations thereof, (e.g., embodiments of R³) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R³ is hydrogen. In embodiments of the formulae above, R³ is —CN. In embodiments of the formulae above, R³ is C₁₋₆alkyl optionally substituted with one, two, or three R²⁰. In embodiments of the formulae above, R³ is C₂₋₆alkenyl optionally substituted with one, two, or three R^{20b}. In embodiments of the formulae above, R³ is C₂₋₆alkynyl optionally substituted with one, two, or three R^{20b}. In embodiments of the formulae above, R³ is C₃₋₁₀cycloalkyl optionally substituted with one, two, or three R^{20b}. In embodiments of the formulae above, R³ is C₂₋₉heterocycloalkyl optionally substituted with one, two, or three R^{20b}. In embodiments of the formulae above, R³ is C₆₋₁₀aryl optionally substituted with one, two, or three R^{20b}. In embodiments of the formulae above, R³ is C₁₋₉heteroaryl optionally substituted with one, two, or three R^{20b}. In embodiments of the formulae above, R³ is selected from —OR¹², —C(O)OR¹², —OC(O)N(R¹²)(R¹³), —C(O)R¹⁵, halogen, and —N(R¹²)(R¹³). In embodiments of the formulae above, R³ is —NH₂.

The individual embodiments herein below, or combinations thereof, (e.g., embodiments of R⁸) are applicable to compounds of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV′), or a pharmaceutically acceptable salt or solvate thereof. In embodiments of the formulae above, R⁸ is hydrogen. In embodiments of the formulae above, R⁸ is halogen. In embodiments of the formulae above, R⁸ is —CN. In embodiments of the formulae above, R⁸ is C₁₋₆ alkyl. In embodiments of the formulae above, R⁸ is C₂₋₆alkenyl. In embodiments of the formulae above, R⁸ is C₂₋₆alkynyl. In embodiments of the formulae above, R⁸ is C₃₋₁₀cycloalkyl. In embodiments of the formulae above, R⁸ is C₂₋₉heterocycloalkyl. In embodiments of the formulae above, R⁸ is C₆₋₁₀aryl. In embodiments of the formulae above, R⁸ is C₁₋₉heteroaryl.

In embodiments of the formulae above, R⁸ is C₁₋₆alkyl optionally substituted with one, two, or three R^{20c}. In embodiments of the formulae above, R⁸ is C₂₋₆alkenyl optionally substituted with one, two, or three R^{20c}. In embodiments of the formulae above, R⁸ is C₂₋₆alkynyl optionally substituted with one, two, or three R^{20c}. In embodiments of the formulae above, R⁸ is C₃₋₁₀cycloalkyl optionally substituted with one, two, or three R^{20c}. In embodiments of the formulae above, R⁸ is C₂₋₉heterocycloalkyl optionally substituted with one, two, or three R^{2c}. In embodiments of the formulae above, R⁸ is C₆₋₁₀aryl optionally substituted with one, two, or three R^{2c}. In embodiments of the formulae above, R⁸ is C₁₋₉heteroaryl optionally substituted with one, two, or three R^{20c}.

In embodiments of the formulae above, R⁸ is independently selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R^{8a}) are applicable to compounds of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV′), or a pharmaceutically acceptable salt or solvate thereof. In embodiments of the formulae above, R^{8a} is hydrogen. In embodiments of the formulae above, R^{8a} is halogen. In embodiments of the formulae above, R^{8a} is —CN. In embodiments of the formulae above, R^{8a} is C₁₋₆alkyl. In embodiments of the formulae above, R^{8a} is C₂₋₆alkenyl. In embodiments of the formulae above, R^{8a} is C₂₋₆alkynyl. In embodiments of the formulae above, R^{8a} is C₃₋₁₀cycloalkyl. In embodiments of the formulae above, R^{8a} is C₂₋₉heterocycloalkyl. In embodiments of the formulae above, R^{8a} is C₆₋₁₀aryl. In embodiments of the formulae above, R^{8a} is C₁₋₉heteroaryl.

In embodiments of the formulae above, R^{8a} is C₁₋₆alkyl optionally substituted with one, two, or three R^{20c}. In embodiments of the formulae above, R^{8a} is C₂₋₆alkenyl optionally substituted with one, two, or three R^{20c}. In embodiments of the formulae above, R^{8a} is C₂₋₆alkynyl optionally substituted with one, two, or three R^{20c}. In embodiments of the formulae above, R^{8a} is C₃₋₁₀cycloalkyl optionally substituted with one, two, or three R^{20c}. In embodiments of the formulae above, R^{8a} is C₂₋₉heterocycloalkyl optionally substituted with one, two, or three R^{20o}. In embodiments of the formulae above, R^{8a} is C₆₋₁₀aryl optionally substituted with one, two, or three R²¹. In embodiments of the formulae above, R^{8a} is C₁₋₉heteroaryl optionally substituted with one, two, or three R^{20c}.

In embodiments of the formulae above, R^{8a} is independently selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R^{8b}) are applicable to compounds of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV′), or a pharmaceutically acceptable salt or solvate thereof. In embodiments of the formulae above, R^{8b} is hydrogen. In embodiments of the formulae above, R^{8b} is halogen. In embodiments of the formulae above, R^{8b} is —CN. In embodiments of the formulae above, R^{8b} is C₁₋₆ alkyl. In embodiments of the formulae above, R^{8b} is C₂₋₆alkenyl. In embodiments of the formulae above, R^{8b} is C₂₋₆alkynyl. In embodiments of the formulae above, R^{8b} is C₃₋₁₀cycloalkyl. In embodiments of the formulae above, R^{8b} is C₂₋₉heterocycloalkyl. In embodiments of the formulae above, R^{8b} is C₆₋₁₀aryl. In embodiments of the formulae above, R^{8b} is C₁₋₉heteroaryl.

In embodiments of the formulae above, R^{8b} is C₁₋₆alkyl optionally substituted with one, two, or three R^{20c}. In embodiments of the formulae above, R^{8b} is C₂₋₆alkenyl optionally substituted with one, two, or three R^{20c}. In embodiments of the formulae above, R^{8b} is C₂₋₆alkynyl optionally substituted with one, two, or three R^{20c}. In embodiments of the formulae above, R^{8b} is C₃₋₁₀cycloalkyl optionally substituted with one, two, or three R^{20c}. In embodiments of the formulae above, R^{8b} is C₂₋₉heterocycloalkyl optionally substituted with one, two, or three R^{20c}. In embodiments of the formulae above, R^{8b} is C₆₋₁₀aryl optionally substituted with one, two, or three R^{20c}. In embodiments of the formulae above, R^{8b} is C₁₋₉heteroaryl optionally substituted with one, two, or three R^{20c}.

In embodiments of the formulae above, R^{8b} is independently selected from —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R¹⁸) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R¹⁸ is hydrogen. In embodiments of the formulae above, R¹⁸ is halogen. In embodiments of the formulae above, R¹⁸ is —CN. In embodiments of the formulae above, R¹⁸ is C₁₋₆alkyl. In embodiments of the formulae above, R¹⁸ is C₂₋₆alkenyl. In embodiments of the formulae above, R¹⁸ is C₂₋₆alkynyl. In embodiments of the formulae above, R¹⁸ is C₃₋₁₀cycloalkyl. In embodiments of the formulae above, R¹⁸ is C₂₋₉heterocycloalkyl. In embodiments of the formulae above, R¹⁸ is C₆₋₁₀aryl. In embodiments of the formulae above, R¹⁸ is C₁₋₉heteroaryl. In embodiments of the formulae above, R¹⁸ is F. In embodiments of the formulae above, R¹⁸ is Cl. In embodiments of the formulae above, R¹⁸ is Br. In embodiments of the formulae above, R¹⁸ is I. In embodiments of the formulae above, R¹⁸ is cyclopropyl. In embodiments of the formulae above, R¹⁸ is CN substituted C₁₋₄alkyl. In embodiments of the formulae above, R¹⁸ is CN substituted propyl. In embodiments of the formulae above, R¹⁸ is CN substituted ethyl. In embodiments of the formulae above, R¹⁸ is CN substituted butyl. In embodiments of the formulae above, R¹⁸ is CN substituted methyl.

In embodiments of the formulae above, R¹⁸ is C₁₋₆alkyl optionally substituted with one, two, or three R^{20h}. In embodiments of the formulae above, R¹⁸ is C₂₋₆alkenyl optionally substituted with one, two, or three R^{20h}. In embodiments of the formulae above, R¹⁸ is C₂₋₆alkynyl optionally substituted with one, two, or three R^{20h}. In embodiments of the formulae above, R¹⁸ is C₃₋₁₀cycloalkyl optionally substituted with one, two, or three R^{20h}. In embodiments of the formulae above, R¹⁸ is C₂₋₉heterocycloalkyl optionally substituted with one, two, or three R^{20h}. In embodiments of the formulae above, R¹⁸ is C₆₋₁₀aryl optionally substituted with one, two, or three R^{20h}. In embodiments of the formulae above, R¹⁸ is C₁₋₉heteroaryl optionally substituted with one, two, or three R^{20h}.

In embodiments of the formulae above, R¹⁸ is independently selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R^{18a}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R^{18a} is hydrogen. In embodiments of the formulae above, R^{18a} is halogen. In embodiments of the formulae above, R^{18a} is —CN. In embodiments of the formulae above, R^{18a} is C₁₋₆alkyl. In embodiments of the formulae above, R^{18a} is C₂₋₆alkenyl. In embodiments of the formulae above, R^{18a} is C₂₋₆alkynyl. In embodiments of the formulae above, R^{18a} is C₃₋₁₀cycloalkyl. In embodiments of the formulae above, R^{18a} is C₂₋₉heterocycloalkyl. In embodiments of the formulae above, R^{18a} is C₆₋₁₀aryl. In embodiments of the formulae above, R^{18a} is C₁₋₉heteroaryl.

In embodiments of the formulae above, R^{18a} is C₁₋₆alkyl optionally substituted with one, two, or three R^{20h}. In embodiments of the formulae above, R^{18a} is C₂₋₆alkenyl optionally substituted with one, two, or three R^{20h}. In embodiments of the formulae above, R^{18a} is C₂₋₆alkynyl optionally substituted with one, two, or three R^{20h}. In embodiments of the formulae above, R^{18a} is C₃₋₁₀cycloalkyl optionally substituted with one, two, or three R^{20h}. In embodiments of the formulae above, R^{18a} is C₂₋₉heterocycloalkyl optionally substituted with one, two, or three R^{20h}. In embodiments of the formulae above, R^{18a} is C₆₋₁₀aryl optionally substituted with one, two, or three R^{20h}. In embodiments of the formulae above, R^{18a} is C₁₋₉heteroaryl optionally substituted with one, two, or three R^{20h}.

In embodiments of the formulae above, R^{18a} is independently selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R^{18b}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R^{18b} is hydrogen. In embodiments of the formulae above, R^{18b} is halogen. In embodiments of the formulae above, R^{18b} is —CN. In embodiments of the formulae above, R^{18b} is C₁₋₆alkyl. In embodiments of the formulae above, R^{18b} is C₂₋₆alkenyl. In embodiments of the formulae above, R^{18b} is C₂₋₆alkynyl. In embodiments of the formulae above, R^{18b} is C₃₋₁₀cycloalkyl. In embodiments of the formulae above, R^{18b} is C₂₋₉heterocycloalkyl. In embodiments of the formulae above, R^{18b} is C₆₋₁₀aryl. In embodiments of the formulae above, R^{18b} is C₁₋₉heteroaryl.

In embodiments of the formulae above, R^{18b} is C₁₋₆alkyl optionally substituted with one, two, or three R^{20h}. In embodiments of the formulae above, R^{18b} is C₂₋₆alkenyl optionally substituted with one, two, or three R^{20h}. In embodiments of the formulae above, R^{18b} is C₂₋₆alkynyl optionally substituted with one, two, or three R^{20h}. In embodiments of the formulae above, R^{18b} is C₃₋₁₀cycloalkyl optionally substituted with one, two, or three R^{20h}. In embodiments of the formulae above, R^{18b} is C₂₋₉heterocycloalkyl optionally substituted with one, two, or three R^{20h}. In embodiments of the formulae above, R^{18b} is C₆₋₁₀aryl optionally substituted with one, two, or three R^{20h}. In embodiments of the formulae above, R^{18b} is C₁₋₉heteroaryl optionally substituted with one, two, or three R^{20h}.

In embodiments of the formulae above, R^{18b} is independently selected from —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R¹⁶) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R¹⁶ is hydrogen. In embodiments of the formulae above, R¹⁶ is halogen. In embodiments of the formulae above, R¹⁶ is —CN. In embodiments of the formulae above, R¹⁶ is C₁₋₆alkyl. In embodiments of the formulae above, R¹⁶ is C₂₋₆alkenyl. In embodiments of the formulae above, R¹⁶ is C₂₋₆alkynyl. In embodiments of the formulae above, R¹⁶ is C₃₋₁₀cycloalkyl. In embodiments of the formulae above, R¹⁶ is C₂₋₉heterocycloalkyl. In embodiments of the formulae above, R¹⁶ is C₆₋₁₀aryl. In embodiments of the formulae above, R¹⁶ is C₁₋₆heteroaryl. In embodiments of the formulae above, R¹⁶ is F. In embodiments of the formulae above, R¹⁶ is Cl. In embodiments of the formulae above, R¹⁶ is Br. In embodiments of the formulae above, R¹⁶ is I.

In embodiments of the formulae above, R¹⁶ is C₁₋₆alkyl optionally substituted with one, two, or three R^{20g}. In embodiments of the formulae above, R¹⁶ is C₂₋₆alkenyl optionally substituted with one, two, or three R^{20g}. In embodiments of the formulae above, R¹⁶ is C₂₋₆alkynyl optionally substituted with one, two, or three R^{20g}. In embodiments of the formulae above, R¹⁶ is C₃₋₁₀cycloalkyl optionally substituted with one, two, or three R^{20g}. In embodiments of the formulae above, R¹⁶ is C₂₋₉heterocycloalkyl optionally substituted with one, two, or three R^{20g}. In embodiments of the formulae above, R¹⁶ is C₆₋₁₀aryl optionally substituted with one, two, or three R^{20g}. In embodiments of the formulae above, R¹⁶ is C₁₋₉heteroaryl optionally substituted with one, two, or three R^{20g}.

In embodiments of the formulae above, R¹⁶ is independently selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R¹⁶) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R¹⁶ is hydrogen. In embodiments of the formulae above, R^{16a} is halogen. In embodiments of the formulae above, R¹⁶ is —CN. In embodiments of the formulae above, R^{16a} is C₁₋₆alkyl. In embodiments of the formulae above, R^{16a} is C₂₋₆alkenyl. In embodiments of the formulae above, R¹⁶ is C₂₋₆alkynyl. In embodiments of the formulae above, R^{16a} is C₃₋₁₀cycloalkyl. In embodiments of the formulae above, R^{16a} is C₂₋₉heterocycloalkyl. In embodiments of the formulae above, R^{16a} is C₆₋₁₀aryl. In embodiments of the formulae above, R^{16a} is C₁₋₉heteroaryl.

In embodiments of the formulae above, R^{16a} is C₁₋₆alkyl optionally substituted with one, two, or three R^{20g}. In embodiments of the formulae above, R^{16a} is C₂₋₆alkenyl optionally substituted with one, two, or three R^{20g}. In embodiments of the formulae above, R^{16a} is C₂₋₆alkynyl optionally substituted with one, two, or three R^{20g}. In embodiments of the formulae above, R^{16a} is C₃₋₁₀cycloalkyl optionally substituted with one, two, or three R^{20g}. In embodiments of the formulae above, R^{16a} is C₂₋₉heterocycloalkyl optionally substituted with one, two, or three R^{20g}. In embodiments of the formulae above, R^{16a} is C₆₋₁₀aryl optionally substituted with one, two, or three R^{20g}. In embodiments of the formulae above, R^{16a} is C₁₋₉heteroaryl optionally substituted with one, two, or three R^{20g}.

In embodiments of the formulae above, R^{16a} is independently selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³) —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R^{6b}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above, R^{16b} is hydrogen. In embodiments of the formulae above, R^{16b} is halogen. In embodiments of the formulae above, R^{16b} is —CN. In embodiments of the formulae above, R^{16b} is C₁₋₆alkyl. In embodiments of the formulae above, R^{16b} is C₂₋₆alkenyl. In embodiments of the formulae above, R^{16b} is C₂₋₆alkynyl. In embodiments of the formulae above, R^{16b} is C₃₋₁₀cycloalkyl. In embodiments of the formulae above, R^{16b} is C₂₋₉heterocycloalkyl. In embodiments of the formulae above, R^{16b} is C₆₋₁₀aryl. In embodiments of the formulae above, R^{16b} is C₁₋₉heteroaryl.

In embodiments of the formulae above, R^{16b} is C₁₋₆alkyl optionally substituted with one, two, or three R^{20g}. In embodiments of the formulae above, R^{16b} is C₂₋₆alkenyl optionally substituted with one, two, or three R^{20g}. In embodiments of the formulae above, R^{16b} is C₂₋₆alkynyl optionally substituted with one, two, or three R^{20g}. In embodiments of the formulae above, R^{16b} is C₃₋₁₀cycloalkyl optionally substituted with one, two, or three R^{20g}. In embodiments of the formulae above, R^{16b} is C₂₋₉heterocycloalkyl optionally substituted with one, two, or three R^{20g}. In embodiments of the formulae above, R^{16b} is C₆₋₁₀aryl optionally substituted with one, two, or three R^{20g}. In embodiments of the formulae above, R^{16b} is C₁₋₉heteroaryl optionally substituted with one, two, or three R^{20g}.

In embodiments of the formulae above, R^{16b} is independently selected from —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³).

The individual embodiments herein below, or combinations thereof, (e.g., embodiments of R¹², R^{12b}, R^{12c}, or R¹²) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, R¹² is independently C₁₋₆alkyl. In embodiments of the formulae above, R¹² is independently C₂₋₆alkenyl. In embodiments of the formulae above, R¹² is independently C₂₋₆alkynyl. In embodiments of the formulae above, R¹² is independently C₃₋₁₀cycloalkyl. In embodiments of the formulae above, R¹² is independently —CH₂—C₃₋₁₀cycloalkyl. In embodiments of the formulae above, R¹² is independently C₂₋₉heterocycloalkyl. In embodiments of the formulae above, R¹² is independently —CH₂—C₂₋₉heterocycloalkyl. In embodiments of the formulae above, R¹² is independently C₆₋₁₀aryl. In embodiments of the formulae above, R¹² is independently —CH₂—C₆₋₁₀aryl. In embodiments of the formulae above, R¹² is independently —CH₂—C₁₋₉heteroaryl. In embodiments of the formulae above, R¹² is independently C₁₋₉heteroaryl

In embodiments of the formulae above, R¹² is independently C₁₋₆alkyl optionally substituted with one, two, or three R^{20d}. In embodiments of the formulae above, R¹² is independently C₂₋₆alkenyl optionally substituted with one, two, or three R^{20d}. In embodiments of the formulae above, R¹² is independently C₂₋₆alkynyl optionally substituted with one, two, or three R^{20d}. In embodiments of the formulae above, R¹² is independently C₃₋₁₀cycloalkyl optionally substituted with one, two, or three R^{20d}. In embodiments of the formulae above, R¹² is independently —CH₂—C₃₋₁₀cycloalkyl optionally substituted with one, two, or three R^{20d}. In embodiments of the formulae above, R¹² is independently C₂₋₉heterocycloalkyl optionally substituted with one, two, or three R^{20d}. In embodiments of the formulae above, R¹² is independently —CH₂—C₂₋₉heterocycloalkyl optionally substituted with one, two, or three R^{20d}. In embodiments of the formulae above, R¹² is independently C₆₋₁₀aryl optionally substituted with one, two, or three R^{20d}. In embodiments of the formulae above, R¹² is independently —CH₂—C₆₋₁₀aryl optionally substituted with one, two, or three R^{20d}. In embodiments of the formulae above, R¹² is independently —CH₂—C₁₋₉heteroaryl optionally substituted with one, two, or three R^{20d}. In embodiments of the formulae above, R¹² is independently C₁₋₉heteroaryl. In additional embodiments, R¹² is independently hydrogen.

In embodiments of the formulae above, R¹² is independently C₂₋₉heterocycloalkyl optionally substituted with one, two, or three R^{20d}. In embodiments of the formulae above, R¹² is independently (monocyclic C₂₋₈heterocycloalkyl) optionally substituted with one, two, or three R^{20d}. In embodiments of the formulae above, R¹² is independently (monocyclic C₃₋₅heterocycloalkyl) optionally substituted with one, two, or three R^{20d}. In embodiments of the formulae above, R¹² is independently (spirocyclic C₂₋₁₁heterocycloalkyl) optionally substituted with one, two, or three R^{20d}. In embodiments of the formulae above, R¹² is independently (spirocyclic C₃₋₁₀heterocycloalkyl) optionally substituted with one, two, or three R^{20d}. In embodiments of the formulae above, R¹² is independently (fused C₂₋₁₁heterocycloalkyl) optionally substituted with one, two, or three R². In embodiments of the formulae above, R¹² is independently (spirocyclic C₆₋₈heterocycloalkyl) optionally substituted with one, two, or three R^{20d}.

In embodiments of the formulae above, R¹² is independently methylene optionally substituted with one or two R^{20d}. In embodiments of the formulae above, R¹² is independently methylene. In embodiments of the formulae above, R¹² is independently ethylene optionally substituted with one, two, or three R^{20d}. In embodiments of the formulae above, R¹² is independently ethylene. In embodiments of the formulae above, R¹² is independently propylene optionally substituted with one, two, or three R^{20d} In embodiments of the formulae above, R¹² is independently propylene. In embodiments of the formulae above, R¹² is independently —CH₂—C₂₋₉heterocycloalkyl optionally substituted with one, two, or three R²¹. In embodiments of the formulae above, R¹² is independently —CH₂-(monocyclic C₂₋₈heterocycloalkyl) optionally substituted with one, two, or three R². In embodiments of the formulae above, R¹² is independently —CH₂-(monocyclic C₃₋₅heterocycloalkyl) optionally substituted with one, two, or three R²⁰. In embodiments of the formulae above, R¹² is independently —CH₂-(spirocyclic C₂₋₁₁heterocycloalkyl) optionally substituted with one, two, or three R²⁰. In embodiments of the formulae above, R¹² is independently —CH₂-(spirocyclic C₃₋₆heterocycloalkyl) optionally substituted with one, two, or three R^{20d}. In embodiments of the formulae above, R¹² is independently —CH₂-(fused C₂₋₁₁heterocycloalkyl) optionally substituted with one, two, or three R^{20d}. In embodiments of the formulae above, R¹² is independently —CH₂-(spirocyclic C₆₋₈heterocycloalkyl) optionally substituted with one, two, or three R^{20d}.

In embodiments of the formulae above, R^{12′} is independently hydrogen. In embodiments of the formulae above, R^{12′} is independently C₁₋₆alkyl. In embodiments of the formulae above, R^{12′} is independently C₂₋₆alkenyl. In embodiments of the formulae above, R^{12′} is independently C₂₋₆alkynyl. In embodiments of the formulae above, R^{12′} is independently C₃₋₁₀cycloalkyl. In embodiments of the formulae above, R^{12′} is independently —C(R^{12c})₂—C₃₋₁₀cycloalkyl. In embodiments of the formulae above, R^{12′} is independently C₂₋₉heterocycloalkyl. In embodiments of the formulae above, R^{12′} is independently —C(R^{12c})₂—C₂₋₉heterocycloalkyl. In embodiments of the formulae above, R^{12′} is independently C₆₋₁₀aryl. In embodiments of the formulae above, R^{12′} is independently —C(R^{12c})₂—C₆₋₁₀aryl. In embodiments of the formulae above, R^{12′} is independently —C(R^{12c})₂—C₁₋₉heteroaryl. In embodiments of the formulae above, R^{12′} is independently and C₁₋₉heteroaryl.

In embodiments of the formulae above, R^{12′} is independently C₁₋₆alkyl optionally substituted with one, two, or three R^{20d}. In embodiments of the formulae above, R^{12′} is independently C₂₋₆alkenyl optionally substituted with one, two, or three R^{20d}. In embodiments of the formulae above, R^{12′} is independently C₂₋₆alkynyl optionally substituted with one, two, or three R^{20d}. In embodiments of the formulae above, R^{12′} is independently C₃₋₁₀cycloalkyl optionally substituted with one, two, or three R^{20d}. In embodiments of the formulae above, R^{12′} is independently —C(R^{12c})₂—C₃₋₁₀cycloalkyl optionally substituted with one, two, or three R^{20d}. In embodiments of the formulae above, R^{12′} is independently C₂₋₉heterocycloalkyl optionally substituted with one, two, or three R^{20d}. In embodiments of the formulae above, R^{12′} is independently —C(R^{12c})₂—C₂₋₉heterocycloalkyl optionally substituted with one, two, or three R^{20d}. In embodiments of the formulae above, R^{12′} is independently C₆₋₁₀aryl optionally substituted with one, two, or three R^{20d}. In embodiments of the formulae above, R^{12′} is independently —C(R^{12c})₂—C₆₋₁₀aryl optionally substituted with one, two, or three R^{20d}. In embodiments of the formulae above, R^{12′} is independently —C(R^{12c})₂—C₁₋₉heteroaryl optionally substituted with one, two, or three R^{20d}. In embodiments of the formulae above, R^{12′} is independently C₁₋₉heteroaryl optionally substituted with one, two, or three R^{20d}.

In select embodiments, R^{12b} is independently C₁₋₆alkyl. In select embodiments, R^{12b} is independently C₁₋₆alkyl optionally substituted with one, two, or three R^{20d}. In embodiments of the formulae above, R^{12b} is independently methylene optionally substituted with one or two R^{20d}. In embodiments of the formulae above, R^{12b} is independently methylene. In embodiments of the formulae above, R^{12b} is independently ethylene optionally substituted with one, two, or three R^{20d}. In embodiments of the formulae above, R^{12″} is independently ethylene. In embodiments of the formulae above, R^{12″} is independently propylene optionally substituted with one, two, or three R^{20d}. In embodiments of the formulae above, R^{12″} is independently propylene. In embodiments of the formulae above, R^{12″} is independently —CH₂—C₂₋₉heterocycloalkyl optionally substituted with one, two, or three R^{20d}. In embodiments of the formulae above, R^{12″} is independently —CH₂-(monocyclic C₂₋₈heterocycloalkyl) optionally substituted with one, two, or three R^{20d}. In embodiments of the formulae above, R^{12b} is independently —CH₂-(monocyclic C₃₋₅heterocycloalkyl) optionally substituted with one, two, or three R^{20d}. In embodiments of the formulae above, R^{12b} is independently —CH₂-(spirocyclic C₂₋₁₁heterocycloalkyl) optionally substituted with one, two, or three R^{20d}. In embodiments of the formulae above, R^{12b} is independently —CH₂-(spirocyclic C₃₋₁₁heterocycloalkyl) optionally substituted with one, two, or three R^{20d}. In embodiments of the formulae above, R^{12b} is independently —CH₂-(fused C₂₋₁₁heterocycloalkyl) optionally substituted with one, two, or three R^{20d}. In embodiments of the formulae above, R^{12″} is independently —CH₂-(spirocyclic C₆₋₈heterocycloalkyl) optionally substituted with one, two, or three R^{20d}.

In embodiments of the formulae above, R^{12″} is independently C₂₋₉heterocycloalkyl optionally substituted with one, two, or three R^{20d}. In embodiments of the formulae above, R^{12″} is independently (monocyclic C₂₋₈heterocycloalkyl) optionally substituted with one, two, or three R^{20d}. In embodiments of the formulae above, R^{12b} is independently (monocyclic C₃₋₅heterocycloalkyl) optionally substituted with one, two, or three R^{20d}. In embodiments of the formulae above, R^{12b} is independently (spirocyclic C₂₋₁₁heterocycloalkyl) optionally substituted with one, two, or three R^{20d}. In embodiments of the formulae above, R^{12b} is independently (spirocyclic C₃₋₁₁heterocycloalkyl) optionally substituted with one, two, or three R^{20d}. In embodiments of the formulae above, R^{12b} is independently (fused C₂₋₁₁heterocycloalkyl) optionally substituted with one, two, or three R^{20d}. In embodiments of the formulae above, R^{12b} is independently (spirocyclic C₆₋₈heterocycloalkyl) optionally substituted with one, two, or three R^{20d}.

In embodiments of the formulae above, R^{12b} is independently C₂₋₆alkenyl. In embodiments of the formulae above, R^{12b} is independently C₂₋₆alkynyl. In embodiments of the formulae above, R^{12b} is independently C₃₋₁₀cycloalkyl. In embodiments of the formulae above, R^{12b} is independently —CH₂—C₃₋₁₀cycloalkyl. In embodiments of the formulae above, R^{12b} is independently C₂₋₉heterocycloalkyl. In embodiments of the formulae above, R^{12b} is independently —CH₂—C₂₋₉heterocycloalkyl. In embodiments of the formulae above, R^{12b} is independently C₆₋₁₀aryl. In embodiments of the formulae above, R^{12b} is independently —CH₂—C₆₋₁₀aryl. In embodiments of the formulae above, R^{12b} is independently —CH₂—C₁₋₉heteroaryl. In embodiments of the formulae above, R^{12b} is independently C₁₋₉heteroaryl

In embodiments of the formulae above, R^{12b} is independently C₂₋₆alkenyl optionally substituted with one, two, or three R^{20d}. In embodiments of the formulae above, R^{12b} is independently C₂₋₆alkynyl optionally substituted with one, two, or three R^{20d}. In embodiments of the formulae above, R^{12b} is independently C₃₋₁₀cycloalkyl optionally substituted with one, two, or three R^{20d}. In embodiments of the formulae above, R^{12b} is independently —CH₂—C₃₋₁₀cycloalkyl optionally substituted with one, two, or three R^{20d}. In embodiments of the formulae above, R^{12b} is independently C₂₋₉heterocycloalkyl optionally substituted with one, two, or three R^{20d}. In embodiments of the formulae above, R^{12b} is independently —CH₂—C₂₋₉heterocycloalkyl optionally substituted with one, two, or three R^{20d}. In embodiments of the formulae above, R^{12b} is independently C₆₋₁₀aryl optionally substituted with one, two, or three R^{20d}. In embodiments of the formulae above, R^{12b} is independently —CH₂—C₆₋₁₀aryl optionally substituted with one, two, or three R^{20d}. In embodiments of the formulae above, R^{12b} is independently —CH₂—C₁₋₉heteroaryl optionally substituted with one, two, or three R^{20d}. In embodiments of the formulae above, R^{12b} is independently C₁₋₉heteroaryl. In additional embodiments, R^{12b} is independently hydrogen.

In embodiments of the formulae above, each R^{12c} is independently hydrogen. In embodiments of the formulae above, each R^{12c} is independently halogen. In embodiments of the formulae above, each R^{12c} is independently oxo. In embodiments of the formulae above, each R^{12c} is independently —CN. In embodiments of the formulae above, each R^{12c} is independently C₁₋₆alkyl. In embodiments of the formulae above, each R^{12c} is independently C₂₋₆alkenyl. In embodiments of the formulae above, each R^{12c} is independently C₂₋₆alkynyl. In embodiments of the formulae above, each R^{12c} is independently C₃₋₁₀cycloalkyl. In embodiments of the formulae above, each R^{12c} is independently —CH₂—C₃₋₁₀cycloalkyl. In embodiments of the formulae above, each R^{12e} is independently C₂₋₉heterocycloalkyl. In embodiments of the formulae above, each R^{12c} is independently —CH₂—C₂₋₉heterocycloalkyl. In embodiments of the formulae above, each R^{12c} is independently C₆₋₁₀aryl. In embodiments of the formulae above, each R^{12c} is independently —CH₂—C₆₋₁₀aryl. In embodiments of the formulae above, each R^{12c} is independently —CH₂—C₁₋₉heteroaryl. In embodiments of the formulae above, each R^{12c} is independently C₁₋₉heteroaryl. In embodiments of the formulae above, each R^{12c} is independently —OR²¹. In embodiments of the formulae above, each R^{12c} is independently —SR²¹. In embodiments of the formulae above, each R^{12c} is independently —N(R²²)(R²³). In embodiments of the formulae above, each R^{12c} is independently —C(O)OR²². In embodiments of the formulae above, each R^{12c} is independently —C(O)N(R²²)(R²³). In embodiments of the formulae above, each R^{12c} is independently —C(O)C(O)N(R²²)(R²³). In embodiments of the formulae above, each R^{12c} is independently —OC(O)N(R²²)(R²³). In embodiments of the formulae above, each R^{12c} is independently —N(R²⁴)C(O)N(R²²)(R²³). In embodiments of the formulae above, each R^{12c} is independently —N(R¹)C(O)OR²⁵. In embodiments of the formulae above, each R^{12c} is independently —N(R²⁴)C(O)R²⁵. In embodiments of the formulae above, each R^{12c} is independently —N(R²⁴)S(O)₂R²⁵. In embodiments of the formulae above, each R^{12c} is independently —C(O)R²⁵. In embodiments of the formulae above, each R^{12c} is independently —S(O)₂R²⁵. In embodiments of the formulae above, each R^{12c} is independently —S(O)₂N(R²²)(R²³). In embodiments of the formulae above, each R^{12c} is independently —OCH₂C(O)OR²². In embodiments of the formulae above, each R^{12c} is independently —OC(O)R²⁵. In embodiments of the formulae above, each R^{12c} is independently C₁₋₆alkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently C₂₋₆alkenyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently C₂₋₆alkynyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently C₃₋₁₀cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently —CH₂—C₃₋₁₀cycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(Ru)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently —CH₂—C₂₋₉heterocycloalkyl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, each R^{12e} is independently —CH₂—C₆₋₁₀aryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently —CH₂—C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently C₁₋₉heteroaryl substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(Ru)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, each R^{12c} is independently methyl. In embodiments of the formulae above, each R^{12c} is independently ethyl. In embodiments of the formulae above, each R^{12c} is independently propyl.

The individual embodiments herein below, or combinations thereof, (e.g., embodiments of R^{12″}) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (IV-3), (IV-4), (IV), or (IV′), or a pharmaceutically acceptable salt or solvate thereof. In embodiments of the formulae above, R^{12″} is independently C₁₋₆alkyl. In embodiments of the formulae above, R^{12″} is independently C₃₋₆alkyl. In embodiments of the formulae above, R^{12″} is independently C₂₋₆alkenyl. In embodiments of the formulae above, R^{12″} is independently C₂₋₆alkynyl. In embodiments of the formulae above, R^{12″} is independently C₃₋₁₀cycloalkyl. In embodiments of the formulae above, R^{12″} is independently —C(R^{12c})₂—C₃₋₁₀cycloalkyl. In embodiments of the formulae above, R^{12″} is independently C₂₋₉heterocycloalkyl. In embodiments of the formulae above, R^{12″} is independently —C(R^{12c})₂—C₂₋₉heterocycloalkyl. In embodiments of the formulae above, R^{12″} is independently C₆₋₁₀aryl. In embodiments of the formulae above, R^{12″} is independently —C(R^{12c})₂—C₆₋₁₀aryl. In embodiments of the formulae above, R^{12″} is independently —C(R^{12c})₂—C₁₋₉heteroaryl. In embodiments of the formulae above, R^{12″} is independently and C₁₋₉heteroaryl.

In embodiments of the formulae above, R^{12″} is independently C₁₋₆alkyl optionally substituted with one, two, or three R^{20d}. In embodiments of the formulae above, R^{12″} is independently C₃₋₆alkyl optionally substituted with one, two, or three R^{20d}. In embodiments of the formulae above, R^{12″} is independently C₂₋₆alkenyl optionally substituted with one, two, or three R^{20d}. In embodiments of the formulae above, R^{12″} is independently C₂₋₆alkynyl optionally substituted with one, two, or three R^{20d}. In embodiments of the formulae above, R^{12″} is independently C₃₋₁₀cycloalkyl optionally substituted with one, two, or three R^{20d}. In embodiments of the formulae above, R^{12″} is independently —C(R^{12c})₂—C₃₋₁₀cycloalkyl optionally substituted with one, two, or three R^{20d}. In embodiments of the formulae above, R^{12″} is independently C₂₋₉heterocycloalkyl optionally substituted with one, two, or three R^{20d}. In embodiments of the formulae above, R^{12″} is independently —C(R^{12c})₂—C₂₋₉heterocycloalkyl optionally substituted with one, two, or three R^{20d}. In embodiments of the formulae above, R^{12″} is independently C₆₋₁₀aryl optionally substituted with one, two, or three R^{20d}. In embodiments of the formulae above, R^{12″} is independently —C(R^{12c})₂—C₆₋₁₀aryl optionally substituted with one, two, or three R^{20d}. In embodiments of the formulae above, R^{12″} is independently —C(R^{12c})₂—C₁₋₉heteroaryl optionally substituted with one, two, or three R^{20d}. In embodiments of the formulae above, R^{12″} is independently C₁₋₉heteroaryl optionally substituted with one, two, or three R^{20d}.

The individual embodiments herein below, or combinations thereof, (e.g., embodiments of R^{12′″}) are applicable to compounds of Formula (IV), (IV-3), (IV-4), (IV′) or (IVa), or a pharmaceutically acceptable salt or solvate thereof. In embodiments of the formulae above, R^{12′″} is independently C₂₋₆alkyl. In embodiments of the formulae above, R^{12′″} is independently C₃₋₆alkyl. In embodiments of the formulae above, R^{12′″} is independently C₂₋₆alkenyl. In embodiments of the formulae above, R^{12′″} is independently C₂₋₆alkynyl. In embodiments of the formulae above, R^{12′″} is independently C₃₋₁₀cycloalkyl. In embodiments of the formulae above, R^{12′″} is independently —C(R^{12c})₂— C₄₋₁₀cycloalkyl. In embodiments of the formulae above, R^{12′″} is independently C₂₋₉heterocycloalkyl. In embodiments of the formulae above, R^{12′″} is independently —C(R^{12c})₂—C₂₋₉heterocycloalkyl. In embodiments of the formulae above, R^{12′″} is independently C₆₋₁₀aryl. In embodiments of the formulae above, R^{12′″} is independently —C(R^{12c})₂—C₆₋₁₀aryl. In embodiments of the formulae above, R^{12′″} is independently —C(R^{12c})₂—C₁₋₉heteroaryl. In embodiments of the formulae above, R^{12′″} is independently and C₁₋₉heteroaryl.

In embodiments of the formulae above, R^{12′″} is independently C₂₋₆alkyl optionally substituted with one, two, or three R^{20d}. In embodiments of the formulae above, R^{12′″} is independently C₃₋₆alkyl optionally substituted with one, two, or three R^{20d}. In embodiments of the formulae above, R^{12′″} is independently C₂₋₆alkenyl optionally substituted with one, two, or three R^{20d}. In embodiments of the formulae above, R^{12′″} is independently C₂₋₆alkynyl optionally substituted with one, two, or three R^{20d}. In embodiments of the formulae above, R^{12′″} is independently C₃₋₁₀cycloalkyl optionally substituted with one, two, or three R^{20d}. In embodiments of the formulae above, R^{12′″} is independently —C(R^{12c})₂—C₄₋₁₀cycloalkyl optionally substituted with one, two, or three R^{20d}. In embodiments of the formulae above, R^{12′″} is independently C₂₋₉heterocycloalkyl optionally substituted with one, two, or three R^{20d}. In embodiments of the formulae above, R^{12′″} is independently —C(R^{12c})₂—C₂₋₉heterocycloalkyl optionally substituted with one, two, or three R^{20d}. In embodiments of the formulae above, R^{12′″} is independently C₆₋₁₀aryl optionally substituted with one, two, or three R^{20d}. In embodiments of the formulae above, R^{12′″} is independently —C(R^{12c})₂—C₆₋₁₀aryl optionally substituted with one, two, or three R^{20d}. In embodiments of the formulae above, R^{12′″} is independently —C(R^{12c})₂—C₁-9heteroaryl optionally substituted with one, two, or three R²¹. In embodiments of the formulae above, R^{12′″} is independently C₁₋₉heteroaryl optionally substituted with one, two, or three R^{20d};

The individual embodiments herein below, or combinations thereof, (e.g., embodiments of R^{12′}″″) are applicable to compounds of Formula (IV), (IV-3), (IV-4), (IV′) or (IVa), or a pharmaceutically acceptable salt or solvate thereof. In embodiments of the formulae above, R^{12″″} is independently C₁₋₆alkyl. In embodiments of the formulae above, R^{12″″} is independently C₃₋₆alkyl. In embodiments of the formulae above, R^{12″″} is independently C₂₋₆alkenyl. In embodiments of the formulae above, R^{12″″} is independently C₂₋₆alkynyl. In embodiments of the formulae above, R^{12″″} is independently C₃₋₁₀cycloalkyl. In embodiments of the formulae above, R^{12″″} is independently —C(R^{12c})₂—C₃₋₁₀cycloalkyl. In embodiments of the formulae above, R^{12″″} is independently C₂₋₉heterocycloalkyl. In embodiments of the formulae above, R^{12″″} is independently —C(R^{12c})₂—C₂₋₉heterocycloalkyl. In embodiments of the formulae above, R^{12″″} is independently C₆₋₁₀aryl. In embodiments of the formulae above, R^{12″″} is independently —C(R^{12c})₂—C₆₋₁₀aryl. In embodiments of the formulae above, R^{12″″} is independently —C(R^{12c})₂—C₁₋₉heteroaryl. In embodiments of the formulae above, R^{12″″} is independently and C₁₋₉heteroaryl.

In embodiments of the formulae above, R^{12″″} is independently C₁₋₆alkyl optionally substituted with one, two, or three R^{20a} In embodiments of the formulae above, R^{12″″} is independently C₃₋₆alkyl optionally substituted with one, two, or three R^{20d}. In embodiments of the formulae above, R^{12″″} is independently C₂₋₆alkenyl optionally substituted with one, two, or three R^{20d}. In embodiments of the formulae above, R^{12″″} is independently C₂₋₆alkynyl optionally substituted with one, two, or three R^{20d}. In embodiments of the formulae above, R^{12″″} is independently C₃₋₁₀cycloalkyl optionally substituted with one, two, or three R^{20d}. In embodiments of the formulae above, R^{12″″} is independently —C(R^{12c})₂—C₃₋₁₀cycloalkyl optionally substituted with one, two, or three R^{20d}. In embodiments of the formulae above, R^{12″″} is independently C₂₋₉heterocycloalkyl optionally substituted with one, two, or three R^{20d}. In embodiments of the formulae above, R^{12″″} is independently —C(R^{12c})₂—C₂₋₉heterocycloalkyl optionally substituted with one, two, or three R^{20d}. In embodiments of the formulae above, R^{12″″} is independently C₆₋₁₀aryl optionally substituted with one, two, or three R^{20d}. In embodiments of the formulae above, R^{12″″} is independently —C(R^{12c})₂—C₆₋₁₀aryl optionally substituted with one, two, or three R^{20d}. In embodiments of the formulae above, R^{12″″} is independently —C(R^{12c})₂—C₁-9heteroaryl optionally substituted with one, two, or three R²¹. In embodiments of the formulae above, R^{12″″} is independently C₁₋₉heteroaryl optionally substituted with one, two, or three R^{20d};

The individual embodiments herein below, or combinations thereof, (e.g., embodiments of R¹) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above of the formulae above, R¹ is independently C₁₋₆alkyl optionally substituted with one, two, or three R^{20a}. In embodiments of the formulae above, R¹ is independently C₂₋₆alkenyl optionally substituted with one, two, or three R^{20a}. In embodiments of the formulae above, R¹ is independently C₂₋₆alkynyl optionally substituted with one, two, or three R^{20a}. In embodiments of the formulae above, R¹ is independently C₃₋₁₀cycloalkyl optionally substituted with one, two, or three R^{20a}. In embodiments of the formulae above, R¹ is independently —CH₂—C₃₋₁₀cycloalkyl optionally substituted with one, two, or three R^{20a}. In embodiments of the formulae above, R¹ is independently C₂₋₉heterocycloalkyl optionally substituted with one, two, or three R^{20a}. In embodiments of the formulae above, R¹ is independently —CH₂—C₂₋₉heterocycloalkyl optionally substituted with one, two, or three R^{20a}In embodiments of the formulae above, R¹ is independently C₆₋₁₀aryl optionally substituted with one, two, or three R^{20a}. In embodiments of the formulae above, R¹ is independently —CH₂—C₆₋₁₀aryl optionally substituted with one, two, or three R^{20a}. In embodiments of the formulae above, R¹ is independently —CH₂—C₁₋₉heteroaryl optionally substituted with one, two, or three R^{20a}. In embodiments of the formulae above, R¹ is independently C₁₋₉heteroaryl. In additional embodiments, R¹ is independently hydrogen.

In embodiments of the formulae above, R¹ is independently C₂₋₉heterocycloalkyl optionally substituted with one, two, or three R^{20a}. In embodiments of the formulae above, R¹ is independently (monocyclic C₂₋₈heterocycloalkyl) optionally substituted with one, two, or three R^{20a}. In embodiments of the formulae above, R¹ is independently (monocyclic C₃₋₅heterocycloalkyl) optionally substituted with one, two, or three R^{20a}. In embodiments of the formulae above, R¹ is independently (spirocyclic C₂₋₁₁heterocycloalkyl) optionally substituted with one, two, or three R^{20a}. In embodiments of the formulae above, R¹ is independently (spirocyclic C₃₋₅heterocycloalkyl) optionally substituted with one, two, or three R^{20a}. In embodiments of the formulae above, R¹ is independently (fused C₂₋₁₁heterocycloalkyl) optionally substituted with one, two, or three R^{20a}. In embodiments of the formulae above, R¹ is independently (spirocyclic C₆₋₈heterocycloalkyl) optionally substituted with one, two, or three R^{20a}.

In embodiments of the formulae above, R¹ is independently methyl optionally substituted with one or two R^{20a}. In embodiments of the formulae above, R¹ is independently methyl. In embodiments of the formulae above, R¹ is independently ethyl optionally substituted with one, two, or three R^{20a}. In embodiments of the formulae above, R¹ is independently ethyl. In embodiments of the formulae above, R¹ is independently propyl optionally substituted with one, two, or three R^{20a}. In embodiments of the formulae above, R¹ is independently propyl. In embodiments of the formulae above, R¹ is independently —CH₂—C₂₋₉heterocycloalkyl optionally substituted with one, two, or three R^{20a}. In embodiments of the formulae above, R¹ is independently —CH₂-(monocyclic C₂₋₈heterocycloalkyl) optionally substituted with one, two, or three R^{20a}. In embodiments of the formulae above, R¹ is independently —CH₂-(monocyclic C₃₋₅heterocycloalkyl) optionally substituted with one, two, or three R^{20a}. In embodiments of the formulae above, R¹ is independently —CH₂-(spirocyclic C₂₋₁₁heterocycloalkyl) optionally substituted with one, two, or three R^{20a}. In embodiments of the formulae above, R¹ is independently —CH₂-(spirocyclic C₃₋₁₁heterocycloalkyl) optionally substituted with one, two, or three R^{20a}. In embodiments of the formulae above, R¹ is independently —CH₂-(fused C₂₋₆heterocycloalkyl) optionally substituted with one, two, or three R^{20a}. In embodiments of the formulae above, R¹ is independently —CH₂-(spirocyclic C₆₋₈heterocycloalkyl) optionally substituted with one, two, or three R^{20a}.

The individual embodiments herein below, or combinations thereof, (e.g., embodiments of R⁴) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above of the formulae above, R⁴ is independently C₁₋₆alkyl. In embodiments of the formulae above, R⁴ is independently C₂₋₆alkenyl. In embodiments of the formulae above, R⁴ is independently C₂₋₆alkynyl. In embodiments of the formulae above, R⁴ is independently C₃₋₁₀cycloalkyl. In embodiments of the formulae above, R⁴ is independently C₂₋₉heterocycloalkyl. In embodiments of the formulae above, R⁴ is independently C₆₋₁₀aryl. In embodiments of the formulae above, R⁴ is independently and C₁₋₉heteroaryl.

In select embodiments, R⁴ is independently C₁₋₆alkyl optionally substituted with one, two, or three R^{20a}. In embodiments of the formulae above, R⁴ is independently methyl optionally substituted with one or two R^{20a}. In embodiments of the formulae above, R⁴ is independently methyl. In embodiments of the formulae above, R⁴ is independently ethyl optionally substituted with one, two, or three R^{20a}. In embodiments of the formulae above, R⁴ is independently ethyl. In embodiments of the formulae above, R⁴ is independently propyl optionally substituted with one, two, or three R^{20a}. In embodiments of the formulae above, R⁴ is independently propyl. In embodiments of the formulae above, R⁴ is independently C₂₋₆alkenyl optionally substituted with one, two, or three R^{20a}. In embodiments of the formulae above, R⁴ is independently C₂₋₆alkynyl optionally substituted with one, two, or three R^{20a}In embodiments of the formulae above, R⁴ is independently C₃₋₁₀cycloalkyl optionally substituted with one, two, or three R^{20a}. In embodiments of the formulae above, R⁴ is independently C₂₋₉heterocycloalkyl optionally substituted with one, two, or three R^{20a}. In embodiments of the formulae above, R⁴ is independently C₆₋₁₀aryl optionally substituted with one, two, or three R^{20a}. In embodiments of the formulae above, R⁴ is independently and C₁₋₉heteroaryl optionally substituted with one, two, or three R^{20a}.

In additional embodiments, R⁴ is independently hydrogen. In additional embodiments, R⁴ is independently halogen. In additional embodiments, R⁴ is independently —CN. In embodiments of the formulae above, R⁴ is —OR¹². In embodiments of the formulae above, R⁴ is —SR¹². In embodiments of the formulae above, R⁴ is —N(R¹²)(R¹³). In embodiments of the formulae above, R⁴ is —C(O)OR¹². In embodiments of the formulae above, R⁴ is —OC(O)N(R¹²)(R¹³). In embodiments of the formulae above, R⁴ is —N(R¹⁴)C(O)N(R¹²)(R¹³). In embodiments of the formulae above, R⁴ is —N(R¹⁴)C(O)OR¹⁵. In embodiments of the formulae above, R⁴ is —N(R¹⁴)S(O)₂R¹⁵. In embodiments of the formulae above, R⁴ is —C(O)R¹⁵. In embodiments of the formulae above, R⁴ is —S(O)R¹⁵. In embodiments of the formulae above, R⁴ is —OC(O)R¹⁵. In embodiments of the formulae above, R⁴ is —C(O)N(R¹²)(R¹³). In embodiments of the formulae above, R⁴ is —C(O)C(O)N(R¹²)(R¹³). In embodiments of the formulae above, R⁴ is —N(R¹⁴)C(O)R¹⁵. In embodiments of the formulae above, R⁴ is —S(O)₂R¹⁵. In embodiments of the formulae above, R⁴ is —S(O)₂N(R¹²)(R¹³). In embodiments of the formulae above, R⁴ is S(═O)(═NH)N(R¹²)(R¹³). In embodiments of the formulae above, R⁴ is —CH₂C(O)N(R¹²)(R¹³). In embodiments of the formulae above, R⁴ is —CH₂N(R¹⁴)C(O)R¹⁵. In embodiments of the formulae above, R⁴ is —CH₂S(O)₂R¹⁵. In embodiments of the formulae above, R⁴ is —CH₂S(O)₂N(R¹²)(R¹³). In embodiments of the formulae above, R⁴ is

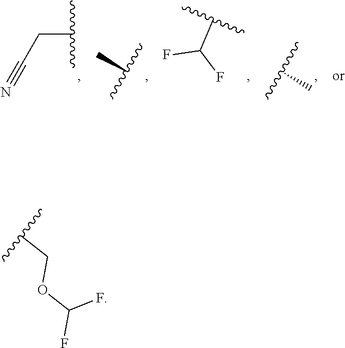

In select embodiments, R² is independently C₁₋₆alkyl. In embodiments of the formulae above, R² is independently C₂₋₆alkenyl. In embodiments of the formulae above, R² is independently C₂₋₆alkynyl. In embodiments of the formulae above, R² is independently C₃₋₁₀cycloalkyl. In embodiments of the formulae above, R² is independently C₂₋₉heterocycloalkyl. In embodiments of the formulae above, R² is independently C₆₋₁₀aryl. In embodiments of the formulae above, R² is independently C₁₋₉heteroaryl.

The individual embodiments herein below, or combinations thereof, (e.g., embodiments of R², R^{2b}, or R^{2c}) are applicable to compounds of Formula (I), (I-3), (I-4), (II-3), (I′), (IVa), (Ia), (Ib), (Ic), (Id), (II), (II′), (II″), (IV-3), (IV-4), (IV), or (IV′), or a pharmaceutically acceptable salt or solvate thereof. In embodiments of the formulae above of the formulae above, R² is independently C₁₋₆alkyl optionally substituted with one, two, or three R². In embodiments of the formulae above, R² is independently methyl optionally substituted with one or two R^{20d}. In embodiments of the formulae above, R² is independently methyl. In embodiments of the formulae above, R² is independently ethyl optionally substituted with one, two, or three R^{20d}. In embodiments of the formulae above, R² is independently ethyl. In embodiments of the formulae above, R² is independently propyl optionally substituted with one, two, or three R^{20d}. In embodiments of the formulae above, R² is independently propyl. In embodiments of the formulae above, R² is independently C₂₋₆alkenyl optionally substituted with one, two, or three R². In embodiments of the formulae above, R² is independently C₂₋₆alkynyl optionally substituted with one, two, or three R². In embodiments of the formulae above, R² is independently C₃₋₁₀cycloalkyl optionally substituted with one, two, or three R²¹. In embodiments of the formulae above, R² is independently C₂₋₉heterocycloalkyl optionally substituted with one, two, or three R^{20d}. In embodiments of the formulae above, R² is independently C₆₋₁₀aryl optionally substituted with one, two, or three R^{20d}. In embodiments of the formulae above, R² is independently and C₁₋₉heteroaryl optionally substituted with one, two, or three R^{20d}.

In additional embodiments, R² is independently halogen. In additional embodiments, R² is independently —CN. In embodiments of the formulae above, R² is —OR^{12′}. In embodiments of the formulae above, R² is —SR^{12′}. In embodiments of the formulae above, R² is —N(R^{12″})(R¹³). In embodiments of the formulae above, R² is —N(R^{12′})(R¹³). In embodiments of the formulae above, R² is —C(O)OR^{12′}. In embodiments of the formulae above, R² is —OC(O)N(R^{12′})(R¹³). In embodiments of the formulae above, R² is —N(R¹⁴)C(O)N(R^{12′})(R¹³). In embodiments of the formulae above, R² is —N(R¹⁴)C(O)OR¹⁵. In embodiments of the formulae above, R² is —N(R¹⁴)S(O)₂R¹⁵. In embodiments of the formulae above, R² is —C(O)R¹⁵. In embodiments of the formulae above, R² is —S(O)R¹⁵. In embodiments of the formulae above, R² is —OC(O)R¹⁵. In embodiments of the formulae above, R² is —C(O)N(R^{12′})(R¹³). In embodiments of the formulae above, R² is —C(O)C(O)N(R^{12′})(R¹³). In embodiments of the formulae above, R² is —N(R¹⁴)C(O)R¹⁵. In embodiments of the formulae above, R² is —S(O)₂R¹⁵. In embodiments of the formulae above, R² is —S(O)₂N(R^{12′})(R¹³). In embodiments of the formulae above, R² is S(═O)(═NH)N(R^{12′})(R¹³). In embodiments of the formulae above, R² is —CH₂C(O)N(R^{12′})(R¹³). In embodiments of the formulae above, R² is —CH₂N(R¹⁴)C(O)R¹⁵. In embodiments of the formulae above, R² is —CH₂S(O)₂R¹⁵. In embodiments of the formulae above, R² is —CH₂S(O)₂N(R^{12′})(R¹³).

In embodiments of the formulae above, R² is independently C₂₋₉heterocycloalkyl optionally substituted with one, two, or three R^{20d}. In embodiments of the formulae above, R^{20d} is independently (monocyclic C₂₋₈heterocycloalkyl) optionally substituted with one, two, or three R^{20d}. In embodiments of the formulae above, R² is independently (monocyclic C₃₋₈heterocycloalkyl) optionally substituted with one, two, or three R^{20d}. In embodiments of the formulae above, R² is independently (spirocyclic C₂₋₁₁heterocycloalkyl) optionally substituted with one, two, or three R^{20d}. In embodiments of the formulae above, R² is independently (spirocyclic C₃₋₈heterocycloalkyl) optionally substituted with one, two, or three R^{20d}. In embodiments of the formulae above, R² is independently (fused C₂₋₁₁heterocycloalkyl) optionally substituted with one, two, or three R^{20d}. In embodiments of the formulae above, R² is independently (spirocyclic C₆₋₈heterocycloalkyl) optionally substituted with one, two, or three R^{20d}. In embodiments of the formulae above, R² is independently —(C₁-C₆alkyl)-R^{12b}. In embodiments of the formulae above, R² is independently, —(C₂₋₆alkenyl)-R^{12b}. In embodiments of the formulae above, R² is independently, —(C₂₋₆alkynyl)-R^{12b}. In embodiments of the formulae above, R² is independently, —(C₃₋₁₀cycloalkyl)-R^{12b}. In embodiments of the formulae above, R² is independently, —(C₂₋₉heterocycloalkyl)-R^{12b}. In embodiments of the formulae above, R² is independently, —(C₆₋₁₀aryl)-R^{12b}. In embodiments of the formulae above, R^{12b} is independently, or —(C₁₋₉heteroaryl)-R^{12b}. In embodiments of the formulae above, R² is independently —(C₁-C₆alkyl)-R^{12b} optionally substituted with one, two, or three R^{20d}. In embodiments of the formulae above, R² is independently, —(C₂₋₆alkenyl)-R^{12b} optionally substituted with one, two, or three R^{20d}. In embodiments of the formulae above, R² is independently, —(C₂₋₆alkynyl)-R^{12b} optionally substituted with one, two, or three R^{20d}. In embodiments of the formulae above, R² is independently, —(C₃₋₁₀cycloalkyl)-R^{12b} optionally substituted with one, two, or three R^{20d}. In embodiments of the formulae above, R is independently, —(C₂₋₉heterocycloalkyl)-R^{12b} optionally substituted with one, two, or three R^{20d}. In embodiments of the formulae above, R² is independently, —(C₆₋₁₀aryl)-R^{12b} optionally substituted with one, two, or three R^{20d}. In embodiments of the formulae above, R² is independently, —(C₁₋₉heteroaryl)-R^{12b} optionally substituted with one, two, or three R^{20d}.

In select embodiments, R^{2c} is independently C₁₋₆alkyl. In embodiments of the formulae above, R^{2c} is independently C₂₋₆alkenyl. In embodiments of the formulae above, R^{2c} is independently C₂₋₆alkynyl. In embodiments of the formulae above, R^{2c} is independently C₃₋₁₀cycloalkyl. In embodiments of the formulae above, R^{2c} is independently C₂₋₉heterocycloalkyl. In embodiments of the formulae above, R^{2c} is independently C₆₋₁₀aryl. In embodiments of the formulae above, R^{2c} is independently C₁₋₉heteroaryl.

In select embodiments, R^{2c} is independently C₁₋₆alkyl optionally substituted with one, two, or three R^{20d}. In embodiments of the formulae above, R^{2c} is independently methyl optionally substituted with one or two R^{20d}. In embodiments of the formulae above, R^{2c} is independently methyl. In embodiments of the formulae above, R^{2c} is independently ethyl optionally substituted with one, two, or three R^{20d}. In embodiments of the formulae above, R^{2c} is independently ethyl. In embodiments of the formulae above, R^{2c} is independently propyl optionally substituted with one, two, or three R^{20d}. In embodiments of the formulae above, R^{2c} is independently propyl. In embodiments of the formulae above, R^{2c} is independently C₂₋₆alkenyl optionally substituted with one, two, or three R². In embodiments of the formulae above, R^{2c} is independently C₂₋₆alkynyl optionally substituted with one, two, or three R^{20d}. In embodiments of the formulae above, R^{2c} is independently C₃₋₁₀cycloalkyl optionally substituted with one, two, or three R². In embodiments of the formulae above, R^{2c} is independently C₂₋₉heterocycloalkyl optionally substituted with one, two, or three R^{20d} In embodiments of the formulae above, R^{2c} is independently C₆₋₁₀aryl optionally substituted with one, two, or three R^{20d}. In embodiments of the formulae above, R^{2c} is independently and C₁₋₉heteroaryl. In additional embodiments, R^{2c} is independently hydrogen.

In additional embodiments, R^{2c} is independently halogen. In additional embodiments, R^{2c} is independently —CN. In embodiments of the formulae above, R^{2c} is —OR¹². In embodiments of the formulae above, R^{2c} is —SR¹². In embodiments of the formulae above, R^{2c} is —N(R¹²)(R¹³). In embodiments of the formulae above, R^{2c} is —C(O)OR¹². In embodiments of the formulae above, R² is —OC(O)N(R¹²)(R¹³). In embodiments of the formulae above, R² is —N(R¹⁴)C(O)N(R¹²)(R¹³). In embodiments of the formulae above, R^{2c} is —N(R¹⁴)C(O)OR¹⁵. In embodiments of the formulae above, R² is —N(R¹⁴)S(O)₂R¹⁵. In embodiments of the formulae above, R^{2c} is —C(O)R¹⁵. In embodiments of the formulae above, R^{2c} is —S(O)R¹⁵. In embodiments of the formulae above, R^{2c} is —OC(O)R¹⁵. In embodiments of the formulae above, R^{2c} is —C(O)N(R¹²)(R¹³). In embodiments of the formulae above, R² is —C(O)C(O)N(R¹²)(R¹³). In embodiments of the formulae above, R² is —N(R¹⁴)C(O)R¹⁵. In embodiments of the formulae above, R^{2c} is —S(O)₂R¹⁵. In embodiments of the formulae above, R^{2c} is —S(O)₂N(R¹²)(R¹³). In embodiments of the formulae above, R^{2c} is S(═O)(═NH)N(R¹²)(R¹³). In embodiments of the formulae above, R^{2c} is —CH₂C(O)N(R¹²)(R¹³). In embodiments of the formulae above, R^{2c} is —CH₂N(R¹⁴)C(O)R¹⁵. In embodiments of the formulae above, R^{2c} is —CH₂S(O)₂R¹⁵. In embodiments of the formulae above, R^{2c} is —CH₂S(O)₂N(R¹²)(R¹³).

In embodiments of the formulae above, R^{2c} is independently C₂₋₉heterocycloalkyl optionally substituted with one, two, or three R². In embodiments of the formulae above, R^{2c} is independently (monocyclic C₂₋₈heterocycloalkyl) optionally substituted with one, two, or three R^{20d}. In embodiments of the formulae above, R^{2c} is independently (monocyclic C₃₋₅heterocycloalkyl) optionally substituted with one, two, or three R^{2c}. In embodiments of the formulae above, R^{2c} is independently (spirocyclic C₂₋₁₁heterocycloalkyl) optionally substituted with one, two, or three R^{20d}. In embodiments of the formulae above, R^{2c} is independently (spirocyclic C₃₋₁₁heterocycloalkyl) optionally substituted with one, two, or three R^{20d}. In embodiments of the formulae above, R^{2c} is independently (fused C₂₋₁₁heterocycloalkyl) optionally substituted with one, two, or three R^{20d}. In embodiments of the formulae above, R^{2c} is independently (spirocyclic C₆₋₈heterocycloalkyl) optionally substituted with one, two, or three R^{20d}. In embodiments of the formulae above, R^{2c} is independently —(C₁-C₆alkyl)-R^{12b}. In embodiments of the formulae above, R^{2c} is independently, —(C₂₋₆alkenyl)-R^{12b}. In embodiments of the formulae above, R^{2c} is independently, —(C₂₋₆alkynyl)-R^{12b}. In embodiments of the formulae above, R^{2c} is independently, —(C₃₋₁₀cycloalkyl)-R^{12b}. In embodiments of the formulae above, R^{2c} is independently, —(C₂₋₉heterocycloalkyl)-R^{12b}. In embodiments of the formulae above, R^{2c} is independently, —(C₆₋₁₀aryl)-R^{12b}. In embodiments of the formulae above, R^{2c} is independently, or —(C₁₋₉heteroaryl)-R^{12b}. In embodiments of the formulae above, R^{2c} is independently —(C₁-C₆alkyl)-R^{12b} optionally substituted with one, two, or three R^{20d}. In embodiments of the formulae above, R^{2c} is independently, —(C₂₋₆alkenyl)-R^{12b} optionally substituted with one, two, or three R^{20d}. In embodiments of the formulae above, R^{2c} is independently, —(C₂₋₆alkynyl)-R^{12b} optionally substituted with one, two, or three R². In embodiments of the formulae above, R^{2c} is independently, —(C₃₋₁₀cycloalkyl)-R^{12b} optionally substituted with one, two, or three R². In embodiments of the formulae above, R^{2c} is independently, —(C₂₋₉heterocycloalkyl)-R^{12b} optionally substituted with one, two, or three R². In embodiments of the formulae above, R^{2c} is independently, —(C₆₋₁₀aryl)-R^{12b} optionally substituted with one, two, or three R^{20d}. In embodiments of the formulae above, R^{2c} is independently, —(C₁₋₉heteroaryl)-R^{12b} optionally substituted with one, two, or three R^{20d}.

In additional embodiments, R^{2b} is independently —CN. In embodiments of the formulae above, R^{2b} is —OR^{12′}. In embodiments of the formulae above, R^{2b} is —SR^{12′}. In embodiments of the formulae above, R^{2b} is —C(O)OR^{12′}. In embodiments of the formulae above, R^{2b} is —OC(O)N(R^{12′})(R¹³). In embodiments of the formulae above, R^{2b} is —C(O)R¹⁵. In embodiments of the formulae above, R^{2b} is —S(O)R¹⁵. In embodiments of the formulae above, R^{2b} is —OC(O)R¹⁵. In embodiments of the formulae above, R^{2b} is —C(O)N(R^{12′})(R¹³). In embodiments of the formulae above, R^{2b} is —C(O)C(O)N(R^{12′})(R¹³). In embodiments of the formulae above, R^{2b} is —S(O)₂R¹⁵. In embodiments of the formulae above, R² is —S(O)₂N(R^{12′})(R¹³). In embodiments of the formulae above, R^{2b} is S(═O)(═NH)N(R^{12′})(R¹³). In embodiments of the formulae above, R^{2b} is —CH₂C(O)N(R^{12′})(R¹³). In embodiments of the formulae above, R^{2b} is —CH₂N(R¹⁴)C(O)R¹⁵. In embodiments of the formulae above, R^{2b} is —CH₂S(O)₂R¹⁵. In embodiments of the formulae above, R^{2b} is —CH₂S(O)₂N(R^{12′})(R¹³).

In select embodiments, R^{2b} is independently C₁₋₆alkyl. In embodiments of the formulae above, R^{2b} is independently C₂₋₆alkenyl. In embodiments of the formulae above, R^{2b} is independently C₂₋₆alkynyl. In embodiments of the formulae above, R^{2b} is independently C₃₋₁₀cycloalkyl. In embodiments of the formulae above, R^{2b} is independently C₂₋₉heterocycloalkyl. In embodiments of the formulae above, R^{2b} is independently C₆₋₁₀aryl. In embodiments of the formulae above, R^{2b} is independently and C₁₋₉heteroaryl.

In select embodiments, R^{2b} is independently C₁₋₆alkyl optionally substituted with one, two, or three R^{20d}. In embodiments of the formulae above, R^{2b} is independently methyl optionally substituted with one or two R². In embodiments of the formulae above, R^{2b} is independently methyl. In embodiments of the formulae above, R^{2b} is independently ethyl optionally substituted with one, two, or three R^{20d}. In embodiments of the formulae above, R^{2b} is independently ethyl. In embodiments of the formulae above, R^{2b} is independently propyl optionally substituted with one, two, or three R^{20d}. In embodiments of the formulae above, R^{2b} is independently propyl. In embodiments of the formulae above, R^{2b} is independently C₂₋₆alkenyl optionally substituted with one, two, or three R^{20d}. In embodiments of the formulae above, R^{2b} is independently C₂₋₆alkynyl optionally substituted with one, two, or three R^{20d}. In embodiments of the formulae above, R^{2b} is independently C₃₋₁₀cycloalkyl optionally substituted with one, two, or three R^{20d}. In embodiments of the formulae above, R^{2b} is independently C₂₋₉heterocycloalkyl optionally substituted with one, two, or three R^{20d}. In embodiments of the formulae above, R^{2b} is independently C₆₋₁₀aryl optionally substituted with one, two, or three R^{20d}. In embodiments of the formulae above, R^{2b} is independently and C₁₋₉heteroaryl. In additional embodiments, R^{2b} is independently hydrogen.

In embodiments of the formulae above, R^{2b} is independently C₂₋₉heterocycloalkyl optionally substituted with one, two, or three R^{20d}. In embodiments of the formulae above, R^{2b} is independently (monocyclic C₂₋₈heterocycloalkyl) optionally substituted with one, two, or three R^{20d}. In embodiments of the formulae above, R^{2b} is independently (monocyclic C₃₋₅heterocycloalkyl) optionally substituted with one, two, or three R^{20d}. In embodiments of the formulae above, R^{2b} is independently (spirocyclic C₂₋₁₁heterocycloalkyl) optionally substituted with one, two, or three R^{20d}. In embodiments of the formulae above, R^{2b} is independently (spirocyclic C₃₋₁₁heterocycloalkyl) optionally substituted with one, two, or three R^{20d}. In embodiments of the formulae above, R^{2b} is independently (fused C₂₋₁₁heterocycloalkyl) optionally substituted with one, two, or three R^{20d}. In embodiments of the formulae above, R^{2b} is independently (spirocyclic C₆₋₈heterocycloalkyl) optionally substituted with one, two, or three R^{20d}. In embodiments of the formulae above, R^{2b} is independently —(C₁-C₆alkyl)-R^{12b}. In embodiments of the formulae above, R^{2b} is independently, —(C₂₋₆alkenyl)-R^{12b}. In embodiments of the formulae above, R^{2b} is independently, —(C₂₋₆alkynyl)-R^{12b}. In embodiments of the formulae above, R^{2b} is independently, —(C₃₋₁₀cycloalkyl)-R^{12b}. In embodiments of the formulae above, R^{2b} is independently, —(C₂₋₉heterocycloalkyl)-R^{12b}. In embodiments of the formulae above, R^{2b} is independently, —(C₆₋₁₀aryl)-R^{12b}. In embodiments of the formulae above, R^{2b} is independently, or —(C₁₋₉heteroaryl)-R^{12b}. In embodiments of the formulae above, R^{2b} is independently —(C₁-C₆alkyl)-R^{12b} optionally substituted with one, two, or three R². In embodiments of the formulae above, R^{2b} is independently, —(C₂₋₆alkenyl)-R^{12b} optionally substituted with one, two, or three R². In embodiments of the formulae above, R^{2b} is independently, —(C₂₋₆alkynyl)-R^{12b} optionally substituted with one, two, or three R^{20d}. In embodiments of the formulae above, R^{2b} is independently, —(C₃₋₁₀cycloalkyl)-R^{12b} optionally substituted with one, two, or three R^{20d}. In embodiments of the formulae above, R^{2b} is independently, —(C₂₋₉heterocycloalkyl)-R^{12b} optionally substituted with one, two, or three R^{20d}. In embodiments of the formulae above, R^{2b} is independently, —(C₆₋₁₀aryl)-R^{12b} optionally substituted with one, two, or three R^{20d}. In embodiments of the formulae above, R^{2b} is independently, —(C₁₋₉heteroaryl)-R^{12b} optionally substituted with one, two, or three R².

The individual embodiments herein below, or combinations thereof, (e.g., embodiments of R¹³, R¹⁴, R^{14a}, or R¹⁵) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, each R¹³ is independently hydrogen. In embodiments of the formulae above, each R¹³ is independently C₁₋₆ alkyl. In embodiments of the formulae above, each R¹³ is independently C₁₋₆haloalkyl. In embodiments of the formulae above, each R¹² and R¹³, together with the nitrogen to which they are attached, form a C₂₋₉heterocycloalkyl ring optionally substituted with one, two, or three R^{20e}.

In embodiments of the formulae above, each R¹⁴ is independently hydrogen. In embodiments of the formulae above, each R¹⁴ is independently C₁₋₆alkyl. In embodiments of the formulae above, each R¹⁴ is independently C₁₋₆haloalkyl.

In embodiments of the formulae above, each R^{14a} is independently C₁₋₆alkyl. In embodiments of the formulae above, each R^{14a} is independently C₁₋₆haloalkyl.

In embodiments of the formulae above, each R¹⁵ is independently C₁₋₆alkyl. In embodiments of the formulae above, each R¹⁵ is independently C₂₋₆alkenyl. In embodiments of the formulae above, each R¹⁵ is independently C₂₋₆alkynyl. In embodiments of the formulae above, each R¹⁵ is independently C₃₋₁₀cycloalkyl. In embodiments of the formulae above, each R¹⁵ is independently C₂₋₉heterocycloalkyl. In embodiments of the formulae above, each R¹⁵ is independently C₆₋₁₀aryl. In embodiments of the formulae above, each R¹⁵ is independently C₁₋₉heteroaryl.

In embodiments of the formulae above, each R¹⁵ is independently C₁₋₆alkyl substituted with one, two, or three R^{20f}. In embodiments of the formulae above, each R¹⁵ is independently C₂₋₆alkenyl substituted with one, two, or three R^{20f}. In embodiments of the formulae above, each R¹⁵ is independently C₂₋₆alkynyl substituted with one, two, or three R^{20f}. In embodiments of the formulae above, each R¹⁵ is independently C₃₋₁₀cycloalkyl substituted with one, two, or three R^{20f}. In embodiments of the formulae above, each R¹⁵ is independently C₂₋₉heterocycloalkyl substituted with one, two, or three R^{20f}. In embodiments of the formulae above, each R¹⁵ is independently C₆₋₁₀aryl substituted with one, two, or three R^{20f}. In embodiments of the formulae above, each R¹⁵ is independently C₁₋₉ heteroaryl substituted with one, two, or three R^{20f}. In embodiments of the formulae above, each R¹⁵ is independently ethenyl substituted with one, two, or three R^{20f}. In embodiments of the formulae above, each R¹⁵ is independently propenyl substituted with one, two, or three R^{20f}. In embodiments of the formulae above, each R¹⁵ is independently butenyl substituted with one, two, or three R^{20f}. In embodiments of the formulae above, each R¹⁵ is independently ethenyl. In embodiments of the formulae above, each R¹⁵ is independently propenyl. In embodiments of the formulae above, each R¹⁵ is independently butenyl.

The individual embodiments herein below, or combinations thereof, (e.g., embodiments of R^{20a}, R^{20b}, R^{20c}, R^{20d}, R^{20e}, R^{20f}, R^{20g}, R^{20h}, R²⁰ⁱ, R^{20k}, or R^{20m}) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above of the formulae above. In embodiments of the formulae above, R^{20a} is independently halogen. In embodiments of the formulae above, R^{20a} is independently oxo. In embodiments of the formulae above, R^{20a} is independently —CN. In embodiments of the formulae above, R^{20a} is independently C₁₋₆alkyl. In embodiments of the formulae above, R^{20a} is independently C₂₋₆alkenyl. In embodiments of the formulae above, R^{20a} is independently C₂₋₆ alkynyl. In embodiments of the formulae above, R^{20a} is independently C₃₋₁₀cycloalkyl. In embodiments of the formulae above, R^{20a} is independently —CH₂—C₃₋₁₀cycloalkyl. In embodiments of the formulae above, R^{20a} is independently C₂₋₉heterocycloalkyl. In embodiments of the formulae above, R^{20a} is independently —CH₂—C₂₋₉heterocycloalkyl. In embodiments of the formulae above, R^{20a} is independently C₆₋₁₀aryl. In embodiments of the formulae above, R^{20a} is independently —CH₂—C₆₋₁₀aryl. In embodiments of the formulae above, R^{20a} is independently —CH₂—C₁₋₉heteroaryl. In embodiments of the formulae above, R^{20a} is independently C₁₋₉heteroaryl. In embodiments of the formulae above, R^{20a} is independently —OR²¹. In embodiments of the formulae above, R^{20a} is independently —SR²¹. In embodiments of the formulae above, R^{20a} is independently —N(R²²)(R²³). In embodiments of the formulae above, R^{20a} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20a} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20a} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20a} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20a} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20a} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20a} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20a} is independently —CH₂—C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20a} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20a} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20a} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20a} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20b} is independently halogen. In embodiments of the formulae above, R²¹ is independently oxo. In embodiments of the formulae above, R^{20b} is independently —CN. In embodiments of the formulae above, R^{20b} is independently C₁₋₆alkyl. In embodiments of the formulae above, R^{20b} is independently C₂₋₆alkenyl. In embodiments of the formulae above, R^{20b} is independently C₂₋₆alkynyl. In embodiments of the formulae above, R^{20b} is independently C₃₋₁₀cycloalkyl. In embodiments of the formulae above, R^{20b} is independently —CH₂—C₃₋₁₀cycloalkyl. In embodiments of the formulae above, R^{20b} is independently C₂₋₉heterocycloalkyl. In embodiments of the formulae above, R^{20b} is independently —CH₂—C₂₋₉heterocycloalkyl. In embodiments of the formulae above, R^{20b} is independently C₆₋₁₀aryl. In embodiments of the formulae above, R^{20b} is independently —CH₂—C₆₋₁₀aryl. In embodiments of the formulae above, R^{20b} is independently —CH₂—C₁₋₉heteroaryl. In embodiments of the formulae above, R^{20b} is independently C₁₋₉heteroaryl. In embodiments of the formulae above, R^{20b} is independently —OR²¹. In embodiments of the formulae above, R^{20b} is independently —SR²¹. In embodiments of the formulae above, R^{20b} is independently —N(R²²)(R²³). In embodiments of the formulae above, R^{20b} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20b} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²¹ is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20b} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²¹ is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20b} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20b} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20b} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In embodiments of the formulae above, R²¹ is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20b} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20b} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{2′} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20c} is independently halogen. In embodiments of the formulae above, R^{20c} is independently oxo. In embodiments of the formulae above, R^{20c} is independently —CN. In embodiments of the formulae above, R^{20c} is independently C₁₋₆alkyl. In embodiments of the formulae above, R^{20c} is independently C₂₋₆alkenyl. In embodiments of the formulae above, R^{20c} is independently C₂₋₆alkynyl. In embodiments of the formulae above, R² is independently C₃₋₁₀cycloalkyl. In embodiments of the formulae above, R^{20c} is independently —CH₂—C₃₋₁₀cycloalkyl. In embodiments of the formulae above, R² is independently C₂₋₉heterocycloalkyl. In embodiments of the formulae above, R^{20c} is independently —CH₂—C₂₋₉heterocycloalkyl. In embodiments of the formulae above, R^{20c} is independently C₆₋₁₀aryl. In embodiments of the formulae above, R^{20c} is independently —CH₂—C₆₋₁₀aryl. In embodiments of the formulae above, R^{20c} is independently —CH₂—C₁₋₉heteroaryl. In embodiments of the formulae above, R^{20c} is independently C₁₋₉heteroaryl. In embodiments of the formulae above, R^{20c} is independently —OR²¹. In embodiments of the formulae above, R^{20c} is independently —SR²¹. In embodiments of the formulae above, R^{20c} is independently —N(R²²)(R²³). In embodiments of the formulae above, R^{20c} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R² is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20c} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(Ru)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20c} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20c} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²¹ is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20c} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20c} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²¹ is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20c} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²¹ is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20c} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20d} is independently halogen. In embodiments of the formulae above, R² is independently oxo. In embodiments of the formulae above, R^{20d} is independently —CN. In embodiments of the formulae above, R^{20d} is independently C₁₋₆alkyl. In embodiments of the formulae above, R^{20d} is independently C₂₋₆alkenyl. In embodiments of the formulae above, R^{20d} is independently C₂₋₆alkynyl. In embodiments of the formulae above, R^{20d} is independently C₃₋₁₀cycloalkyl. In embodiments of the formulae above, R^{20d} is independently —CH₂—C₃₋₁₀cycloalkyl. In embodiments of the formulae above, R^{20d} is independently C₂₋₉heterocycloalkyl. In embodiments of the formulae above, R^{20d} is independently —CH₂—C₂₋₉heterocycloalkyl. In embodiments of the formulae above, R^{20d} is independently C₆₋₁₀aryl. In embodiments of the formulae above, R² is independently —CH₂—C₆₋₁₀aryl. In embodiments of the formulae above, R^{20d} is independently —CH₂—C₁₋₉heteroaryl. In embodiments of the formulae above, R^{20d} is independently C₁₋₉heteroaryl. In embodiments of the formulae above, R^{20d} is independently —OR²¹. In embodiments of the formulae above, R^{20d} is independently —SR²¹. In embodiments of the formulae above, R^{20d} is independently —N(R²²)(R²³). In embodiments of the formulae above, R^{20d} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20d} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R² is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20d} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R² is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20d} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20d} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R² is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In embodiments of the formulae above, R^{20d} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20d} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20d} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R² is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R²⁰ⁱ is independently halogen. In embodiments of the formulae above, R²⁰ⁱ is independently oxo. In embodiments of the formulae above, R²⁰ⁱ is independently —CN. In embodiments of the formulae above, R²⁰ⁱ is independently C₁₋₆alkyl. In embodiments of the formulae above, R²⁰ⁱ is independently C₂₋₆alkenyl. In embodiments of the formulae above, R²⁰ⁱ is independently C₂₋₆alkynyl. In embodiments of the formulae above, R²⁰ⁱ is independently C₃₋₁₀cycloalkyl. In embodiments of the formulae above, R^{20e} is independently —CH₂—C₃₋₁₀cycloalkyl. In embodiments of the formulae above, R^{20e} is independently C₂₋₉heterocycloalkyl. In embodiments of the formulae above, R^{20e} is independently —CH₂—C₂₋₉heterocycloalkyl. In embodiments of the formulae above, R^{20e} is independently C₆₋₁₀aryl. In embodiments of the formulae above, R^{20e} is independently —CH₂—C₆₋₁₀aryl. In embodiments of the formulae above, R^{20e} is independently —CH₂—C₁₋₉heteroaryl. In embodiments of the formulae above, R^{20e} is independently C₁₋₉heteroaryl. In embodiments of the formulae above, R^{20e} is independently —OR¹. In embodiments of the formulae above, R^{20e} is independently —SR²¹. In embodiments of the formulae above, R^{20e} is independently —N(R²²)(R²³). In embodiments of the formulae above, R^{20e} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20e} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20e} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20e} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20e} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20e} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20e} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20e} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In embodiments of the formulae above, R^{20e} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20e} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20e} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20e} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R²⁰ is independently halogen. In embodiments of the formulae above, R²⁰ is independently oxo. In embodiments of the formulae above, R²⁰ is independently —CN. In embodiments of the formulae above, R²⁰ is independently C₁₋₆alkyl. In embodiments of the formulae above, R^{20f} is independently C₂₋₆alkenyl. In embodiments of the formulae above, R^{20f} is independently C₂₋₆alkynyl. In embodiments of the formulae above, R^{20f} is independently C₃₋₁₀cycloalkyl. In embodiments of the formulae above, R^{20f} is independently —CH₂—C₃₋₁₀cycloalkyl. In embodiments of the formulae above, R^{20f} is independently C₂₋₉heterocycloalkyl. In embodiments of the formulae above, R^{20f} is independently —CH₂—C₂₋₉heterocycloalkyl. In embodiments of the formulae above, R²⁰ is independently C₆₋₁₀aryl. In embodiments of the formulae above, R^{20f} is independently —CH₂—C₆₋₁₀aryl. In embodiments of the formulae above, R²⁰ is independently —CH₂—C₁₋₉heteroaryl. In embodiments of the formulae above, R²⁰ is independently C₁₋₉heteroaryl. In embodiments of the formulae above, R²⁰ is independently —OR²¹. In embodiments of the formulae above, R²⁰ is independently —SR²¹. In embodiments of the formulae above, R²⁰ is independently —N(R²²)(R²³). In embodiments of the formulae above, R^{20f} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20f} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20f} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20f} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20f} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20f} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20f} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R²⁰ is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20f} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20g} is independently halogen. In embodiments of the formulae above, R^{20g} is independently oxo. In embodiments of the formulae above, R^{20g} is independently —CN. In embodiments of the formulae above, R^{20g} is independently C₁₋₆alkyl. In embodiments of the formulae above, R^{20g} is independently C₂₋₆alkenyl. In embodiments of the formulae above, R^{20g} is independently C₂₋₆alkynyl. In embodiments of the formulae above, R^{20g} is independently C₃₋₁₀cycloalkyl. In embodiments of the formulae above, R^{20g} is independently —CH₂—C₃₋₁₀cycloalkyl. In embodiments of the formulae above, R^{20g} is independently C₂₋₉heterocycloalkyl. In embodiments of the formulae above, R^{20g} is independently —CH₂—C₂₋₉heterocycloalkyl. In embodiments of the formulae above, R^{20g} is independently C₆₋₁₀aryl. In embodiments of the formulae above, R^{20g} is independently —CH₂—C₆₋₁₀aryl. In embodiments of the formulae above, R^{20g} is independently —CH₂—C₁₋₉heteroaryl. In embodiments of the formulae above, R^{20g} is independently C₁₋₉heteroaryl. In embodiments of the formulae above, R^{20g} is independently —OR²¹. In embodiments of the formulae above, R^{20g} is independently —SR²¹. In embodiments of the formulae above, R^{20g} is independently —N(R²²)(R²³). In embodiments of the formulae above, R^{20g} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20g} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20g} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20g} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20g} is independently C₃₋₁₀cycloalkyl optionally substituted with one, two, or three groups independently selected from halogen, oxo, —CN, C₁₋₆alkyl, C₁₋₆haloalkyl, C₁. 6alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20g} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20g} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20g} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20g} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20g} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(Ru)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20g} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20g} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20h} is independently halogen. In embodiments of the formulae above, R^{20h} is independently oxo. In embodiments of the formulae above, R^{20h} is independently —CN. In embodiments of the formulae above, R^{20h} is independently C₁₋₆alkyl. In embodiments of the formulae above, R^{20h} is independently C₂₋₆alkenyl. In embodiments of the formulae above, R^{20h} is independently C₂₋₆alkynyl. In embodiments of the formulae above, R^{20h} is independently C₃₋₁₀cycloalkyl. In embodiments of the formulae above, R^{20h} is independently —CH₂—C₃₋₁₀cycloalkyl. In embodiments of the formulae above, R^{20h} is independently C₂₋₉heterocycloalkyl. In embodiments of the formulae above, R^{20h} is independently —CH₂—C₂₋₉heterocycloalkyl. In embodiments of the formulae above, R^{20h} is independently C₆₋₁₀aryl. In embodiments of the formulae above, R^{20h} is independently —CH₂—C₆₋₁₀aryl. In embodiments of the formulae above, R^{20h} is independently —CH₂—C₁₋₉heteroaryl. In embodiments of the formulae above, R^{20h} is independently C₁₋₉heteroaryl. In embodiments of the formulae above, R^{20h} is independently —OR²¹. In embodiments of the formulae above, R^{20h} is independently —SR²¹. In embodiments of the formulae above, R^{20h} is independently —N(R²²)(R²³). In embodiments of the formulae above, R^{20h} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20h} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{2h} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20h} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(RN)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{2h} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20h} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{2h} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20h} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{2h} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20h} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{2h} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20h} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R²⁰ⁱ is independently halogen. In embodiments of the formulae above, R²⁰ⁱ is independently oxo. In embodiments of the formulae above, R²⁰ⁱ is independently —CN. In embodiments of the formulae above, R²⁰ⁱ is independently C₁₋₆alkyl. In embodiments of the formulae above, R²⁰ⁱ is independently C₂₋₆alkenyl. In embodiments of the formulae above, R²⁰ⁱ is independently C₂₋₆alkynyl. In embodiments of the formulae above, R²⁰ⁱ is independently C₃₋₁₀cycloalkyl. In embodiments of the formulae above, R²⁰ⁱ is independently —CH₂—C₃₋₁₀cycloalkyl. In embodiments of the formulae above, R²⁰ⁱ is independently C₂₋₉heterocycloalkyl. In embodiments of the formulae above, R²⁰ⁱ is independently —CH₂—C₂₋₉heterocycloalkyl. In embodiments of the formulae above, R²⁰ⁱ is independently C₆₋₁₀aryl. In embodiments of the formulae above, R²⁰ⁱ is independently —CH₂—C₆₋₁₀aryl. In embodiments of the formulae above, R²⁰ⁱ is independently —CH₂—C₁₋₉heteroaryl. In embodiments of the formulae above, R²⁰ⁱ is independently C₁₋₉heteroaryl. In embodiments of the formulae above, R²⁰ⁱ is independently —OR²¹. In embodiments of the formulae above, R²⁰ⁱ is independently —SR²¹. In embodiments of the formulae above, R²⁰ⁱ is independently —N(R²²)(R²³). In embodiments of the formulae above, R²⁰ⁱ is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R²⁰ⁱ is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently C₃₋₁₀cycloalkyl optionally substituted with one, two, or three groups independently selected from halogen, oxo, —CN, C₁₋₆alkyl, C₁₋₆haloalkyl, C₁₋₆ alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In embodiments of the formulae above, R²⁰ⁱ is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R² is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R² is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁰ is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20k} is independently halogen. In embodiments of the formulae above, R^{20k} is independently oxo. In embodiments of the formulae above, R^{20k} is independently —CN. In embodiments of the formulae above, R^{20k} is independently C₁₋₆alkyl. In embodiments of the formulae above, R^{20k} is independently C₂₋₆alkenyl. In embodiments of the formulae above, R^{20k} is independently C₂₋₆alkynyl. In embodiments of the formulae above, R^{20k} is independently C₃₋₁₀cycloalkyl. In embodiments of the formulae above, R^{20k} is independently —CH₂—C₃₋₁₀cycloalkyl. In embodiments of the formulae above, R^{20k} is independently C₂₋₉heterocycloalkyl. In embodiments of the formulae above, R^{20k} is independently —CH₂—C₂₋₉heterocycloalkyl. In embodiments of the formulae above, R^{20k} is independently C₆₋₁₀aryl. In embodiments of the formulae above, R^{20k} is independently —CH₂—C₆₋₁₀aryl. In embodiments of the formulae above, R^{20k} is independently —CH₂—C₁₋₉heteroaryl. In embodiments of the formulae above, R^{20k} is independently C₁₋₉heteroaryl. In embodiments of the formulae above, R^{20k} is independently —OR²¹. In embodiments of the formulae above, R^{20k} is independently —SR²¹. In embodiments of the formulae above, R^{20k} is independently —N(R²²)(R²³). In embodiments of the formulae above, R^{20k} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20k} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20k} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20k} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20k} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20k} is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20k} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20k} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), and —OC(O)R²⁵. In embodiments of the formulae above, R^{2k} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20k} is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20k} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20k} is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the formulae above, R^{20m} is independently halogen. In embodiments of the formulae above, R^{20m} is independently oxo. In embodiments of the formulae above, R^{20m} is independently —CN. In embodiments of the formulae above, R^{20m} is independently C₁₋₆alkyl. In embodiments of the formulae above, R² is independently C₂₋₆alkenyl. In embodiments of the formulae above, R^{20m} is independently C₂₋₆alkynyl. In embodiments of the formulae above, R^{20m} is independently C₃₋₁₀cycloalkyl. In embodiments of the formulae above, R^{20m} is independently —CH₂—C₃₋₁₀cycloalkyl. In embodiments of the formulae above, R^{20m} is independently C₂₋₉heterocycloalkyl. In embodiments of the formulae above, R^{20m} is independently —CH₂—C₂₋₉heterocycloalkyl. In embodiments of the formulae above, R^{20m} is independently C₆₋₁₀aryl. In embodiments of the formulae above, R^{20m} is independently —CH₂—C₆₋₁₀aryl. In embodiments of the formulae above, R^{20m} is independently —CH₂—C₁₋₉heteroaryl. In embodiments of the formulae above, R^{20m} is independently C₁₋₉heteroaryl. In embodiments of the formulae above, R^{20m} is independently —OR²¹. In embodiments of the formulae above, R^{20m} is independently —SR²¹. In embodiments of the formulae above, R² is independently —N(R²²)(R²³). In embodiments of the formulae above, R^{20m} is independently selected from —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(Ru)S(O)₂R²⁵, —C(O)R²⁵, —S(O)₂R²⁵, —S(O)₂N(R²²)(R²³), —OCH₂C(O)OR²², and —OC(O)R²⁵. In embodiments of the formulae above, R^{20m} is independently C₁₋₆alkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20m} is independently C₂₋₆alkenyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20m} is independently C₂₋₆alkynyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20m} is independently C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁵ is independently —CH₂—C₃₋₁₀cycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20m} is independently C₂₋₉heterocycloalkyl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20m} is independently —CH₂—C₂₋₉heterocycloalkyl optionally substituted with one, two, or three groups independently selected from halogen, oxo, —CN, C₁₋₆alkyl, C₁₋₆ haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R^{20m} is independently C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R²⁵ is independently —CH₂—C₆₋₁₀aryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²)S(O)₂R²⁵, —C(O)R²⁵, —S(O)₂R²⁵, —S(O)₂N(R²²)(R²³), and —OC(O)R²⁵. In embodiments of the formulae above, R^{20m} is independently —CH₂—C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵. In embodiments of the formulae above, R²⁵ is independently C₁₋₉heteroaryl optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

The individual embodiments herein below, or combinations thereof, (e.g., embodiments of R²¹, R²², R²³, R²⁴, or R²⁵) are applicable to compounds of Formula (I), (I-3), (I-4), (II-3), (I′), (IVa), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), or (IV′), or a pharmaceutically acceptable salt or solvate thereof. In embodiments of the formulae above, each R²¹ is independently H. In embodiments of the formulae above, each R²¹ is independently C₁₋₆alkyl. In embodiments of the formulae above, each R²¹ is independently C₁₋₆haloalkyl. In embodiments of the formulae above, each R²¹ is independently C₂₋₆alkenyl. In embodiments of the formulae above, each R²¹ is independently C₂₋₆alkynyl. In embodiments of the formulae above, each R²¹ is independently C₃₋₁₀cycloalkyl. In embodiments of the formulae above, each R²¹ is independently C₂₋₉heterocycloalkyl. In embodiments of the formulae above, each R²¹ is independently C₆₋₁₀aryl. In embodiments of the formulae above, each R²¹ is independently C₁₋₉heteroaryl.

In embodiments of the formulae above, each R²² is independently H. In embodiments of the formulae above, each R²² is independently C₁₋₆alkyl. In embodiments of the formulae above, each R²² is independently C₁₋₆haloalkyl. In embodiments of the formulae above, each R²² is independently C₂₋₆alkenyl. In embodiments of the formulae above, each R²² is independently C₂₋₆alkynyl. In embodiments of the formulae above, each R²² is independently C₃₋₁₀cycloalkyl. In embodiments of the formulae above, each R²² is independently C₂₋₉heterocycloalkyl. In embodiments of the formulae above, each R²² is independently C₆₋₁₀aryl. In embodiments of the formulae above, each R²² is independently C₁₋₉heteroaryl.

In embodiments of the formulae above, each R²³ is independently H. In embodiments of the formulae above, each R²³ is independently C₁₋₆alkyl.

In embodiments of the formulae above, each R²⁴ is independently H. In embodiments of the formulae above, each R²⁴ is independently C₁₋₆alkyl.

In embodiments of the formulae above, each R²⁵ is independently C₁₋₆alkyl. In embodiments of the formulae above, each R²⁵ is independently C₂₋₆alkenyl. In embodiments of the formulae above, each R²⁵ is independently C₂₋₆alkynyl. In embodiments of the formulae above, each R²⁵ is independently C₃₋₁₀cycloalkyl. In embodiments of the formulae above, each R²⁵ is independently C₂₋₉heterocycloalkyl. In embodiments of the formulae above, each R²⁵ is independently C₆₋₁₀aryl. In embodiments of the formulae above, each R²⁵ is independently C₁₋₉heteroaryl.

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

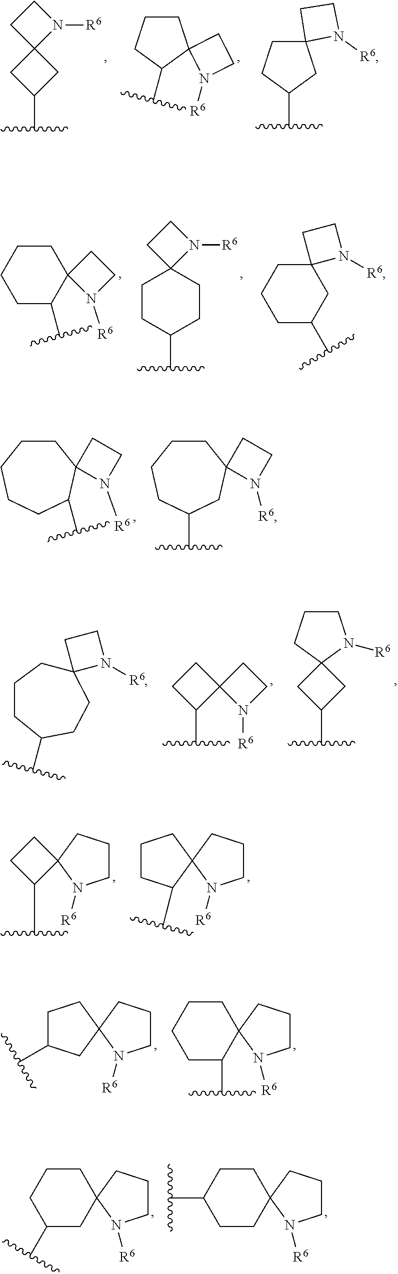

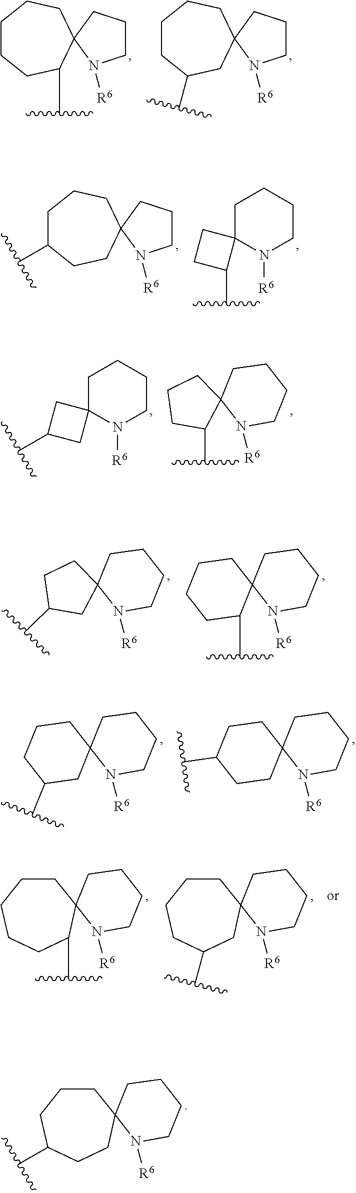

In embodiments of Formula (IV-3), (IV-4), (IV), (IVa), or (IV′), R⁷ is

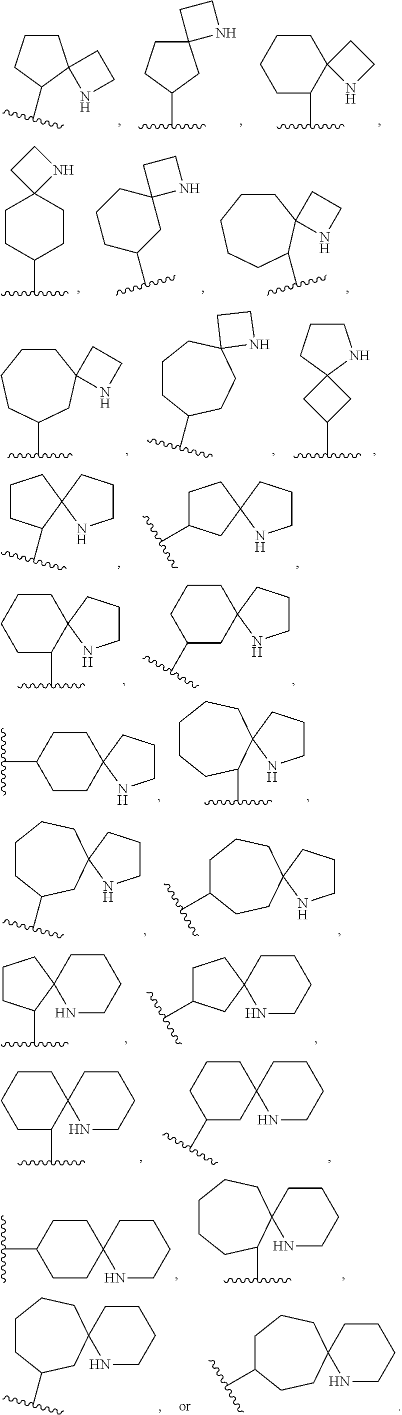

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

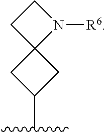

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

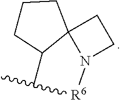

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

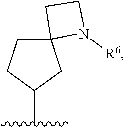

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

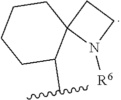

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

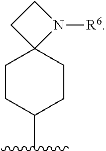

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

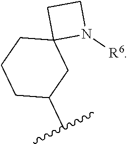

In embodiments of Formula (I), (I-3), (I-4) II-3) (I′) (Ia) (Ib) (Ic) (Id) (Ie) (If) (Ig) (Ii) (Ij) (Ik) (Im) (In) (Io) (Ip) (Iq) (Ir) (II) (II′) or (II″), R⁷ is

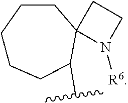

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

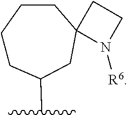

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

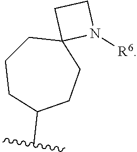

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

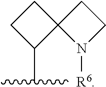

In embodiments of Formula (I), (I-3), (I-4), (II-3), (I′), (Ia), (Ib), (Ic), (Id), (Ie), (If), (Ig), (Ii), (Ij) (Ik), (Im), (In), (Io), (Ip), (Iq), (Ir), (II), (II′), or (II″), R⁷ is

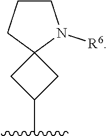

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

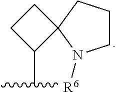

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

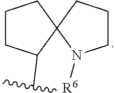

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

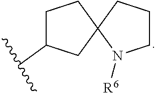

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

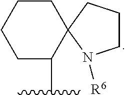

In embodiments of Formula (I), (I-3, (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

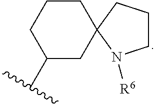

In embodiments of Formula (I), (I-3, (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

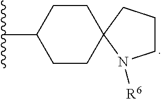

In embodiments of Formula (I), (I-3(Ii)I-4), (I-3), (I′), (Ia), (Ib), (Ic), (Id), (Ie), (If), (I, (Ii), (Ij), (I (m), (In), (Io), (Ip), (Iq), (Ir), (II), (II′), or (II″), R⁷ is

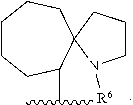

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

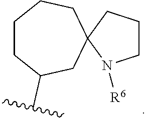

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

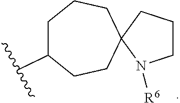

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

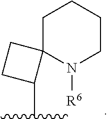

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

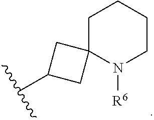

In embodiments of Formula (I), (I-3, (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

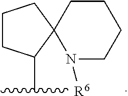

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

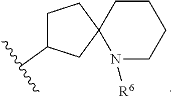

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

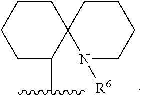

In embodiments of Formula (I), (I-3), (I-4), (II-3), (I′), (II), (Ia), (Ib), (Ic), (Id), (Ie), (If), (Ig), (Ii), (Ij), (Ik), (Im), (In), (Io), (Ip), (Iq), (Ir), (II′), or (II″), R⁷ is

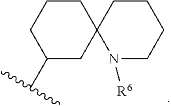

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

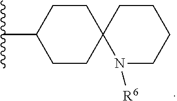

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

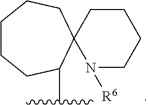

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

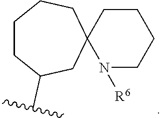

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

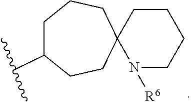

In embodiments of Formula (IV-3), (IV-4), (IV), (IVa), or (IV′), R⁷ is

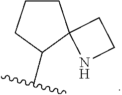 [SMILES: *C1CCCC12CCN2]

In embodiments of Formula (IV-3), (IV-4), (IV), (IVa), or (IV′), R⁷ is

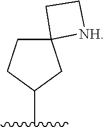 [SMILES: *C1CCC2(CCN2)C1]

In embodiments of Formula (IV-3), (IV-4), (IV), (IVa), or (IV′), R⁷ is

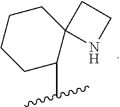 [SMILES: *C1CCCCC12CCN2]

In embodiments of Formula (IV-3), (IV-4), (IV), (IVa), or (IV′), R⁷ is

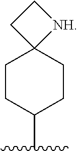 [SMILES: *C1CCC2(CCN2)CC1]

In embodiments of Formula (IV-3), (IV-4), (IV), (IVa), or (IV′), R⁷ is

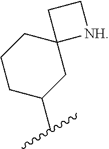 [SMILES: *C1CCCC2(CCN2)C1]

In embodiments of Formula (IV-3), (IV-4), (IV), (IVa), or (IV′), R⁷ is

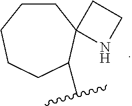 [SMILES: *C1CCCCCC12CCN2]

In embodiments of Formula (IV-3), (IV-4), (IV), (IVa), or (IV′), R⁷ is

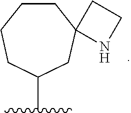 [SMILES: *C1CCCCC2(CCN2)C1]

In embodiments of Formula (IV-3), (IV-4), (IV), (IVa), or (IV′), R⁷ is

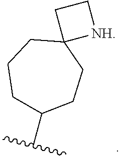 [SMILES: *C1CCCC2(CCN2)CC1]

In embodiments of Formula (IV-3), (IV-4), (IV), (IVa), or (IV′), R⁷ is

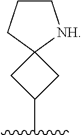 [SMILES: *C1CC2(CCCN2)C1]

In embodiments of Formula (IV-3), (IV-4), (IV), (IVa), or (IV′), R⁷ is

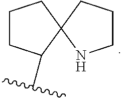 [SMILES: *C1CCCC12CCCN2]

In embodiments of Formula (IV-3), (IV-4), (IV), (IVa), or (IV′), R⁷ is

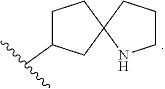 [SMILES: *C1CCC2(CCCN2)C1]

In embodiments of Formula (IV-3), (IV-4), (IV), (IVa), or (IV′), R⁷ is

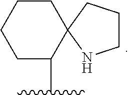 [SMILES: *C1CCCCC12CCCN2]

In embodiments of Formula (IV-3), (IV-4), (IV), (IVa), or (IV′), R⁷ is

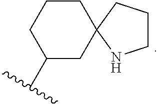 [SMILES: *C1CCCC2(CCCN2)C1]

In embodiments of Formula (IV-3), (IV-4), (IV), (IVa), or (IV′), R⁷ is

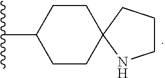 [SMILES: *C1CCC2(CCCN2)CC1]

In embodiments of Formula (IV-3), (IV-4), (IV), (IVa), or (IV′), R⁷ is

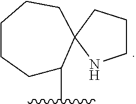 [SMILES: *C1CCCCCC12CCCN2]

In embodiments of Formula (IV-3), (IV-4), (IV), (IVa), or (IV′), R⁷ is

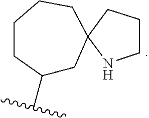 [SMILES: *C1CCCCC2(CCCN2)C1]

In embodiments of Formula (IV-3), (IV-4), (IV), (IVa), or (IV′), R⁷ is

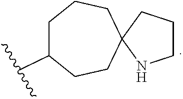 [SMILES: *C1CCCC2(CCCN2)CC1]

In embodiments of Formula (IV-3), (IV-4), (IV), (IVa), or (IV′), R⁷ is

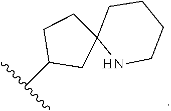 [SMILES: *C1CCC2(CCCCN2)C1]

In embodiments of Formula (IV-3), (IV-4), (IV), (IVa), or (IV′), R⁷ is

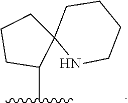 [SMILES: *C1CCCC12CCCCN2]

In embodiments of Formula (IV-3), (IV-4), (IV), (IVa), or (IV′), R⁷ is

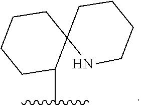 [SMILES: *C1CCCCC12CCCCN2]

In embodiments of Formula (IV-3), (IV-4), (IV), (IVa), or (IV′), R⁷ is

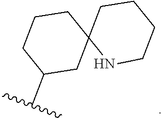 [SMILES: *C1CCCC2(CCCCN2)C1]

In embodiments of Formula (IV-3), (IV-4), (IV), (IVa), or (IV′), R⁷ is

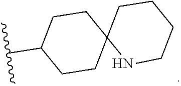 [SMILES: *C1CCC2(CCCCN2)CC1]

In embodiments of Formula (IV-3), (IV-4), (IV), (IVa), or (IV′), R⁷ is

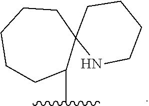 [SMILES: *C1CCCCCC12CCCCN2]

In embodiments of Formula (IV-3), (IV-4), (IV), (IVa), or (IV′), R⁷ is

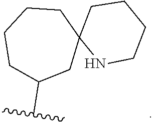 [SMILES: *C1CCCCC2(CCCCN2)C1]

In embodiments of Formula (IV-3), (IV-4), (IV), (IVa), or (IV′), R⁷ is

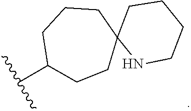 [SMILES: *C1CCCC2(CCCCN2)CC1]

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

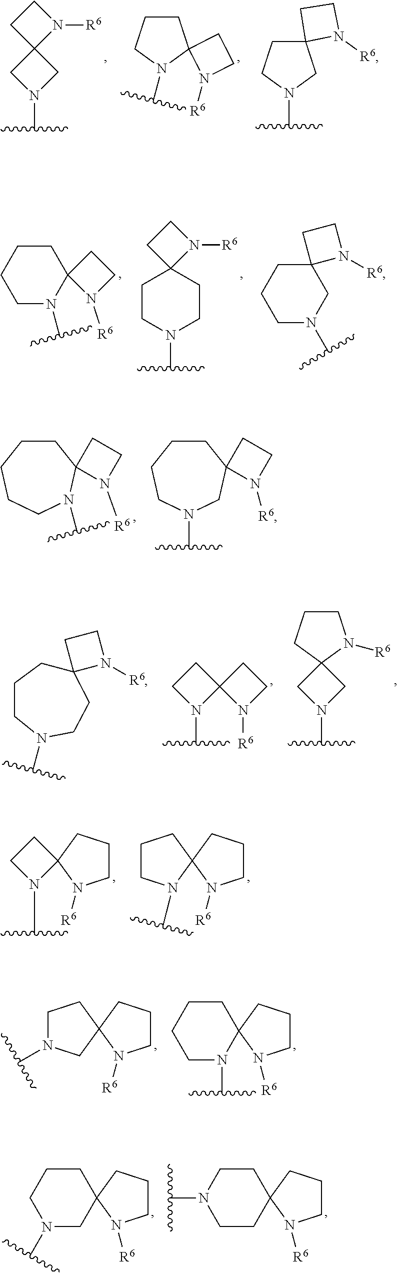

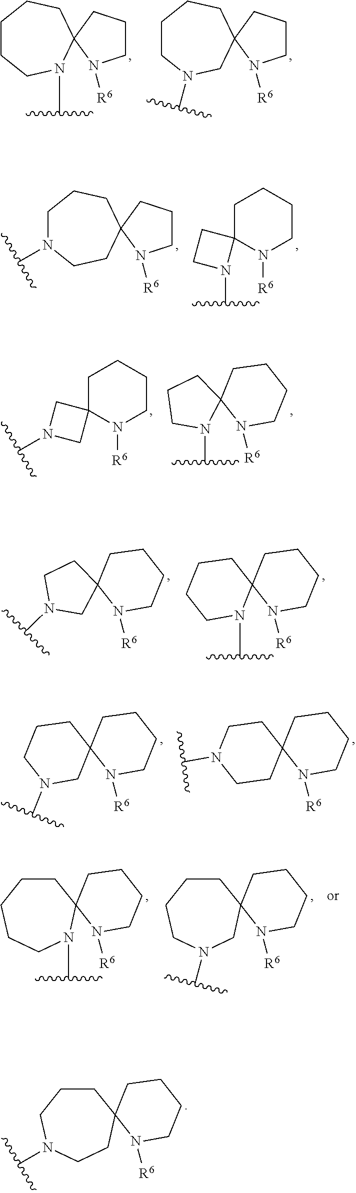

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq) (Ir), (In), (II′), or (II″), R⁷ is selected from

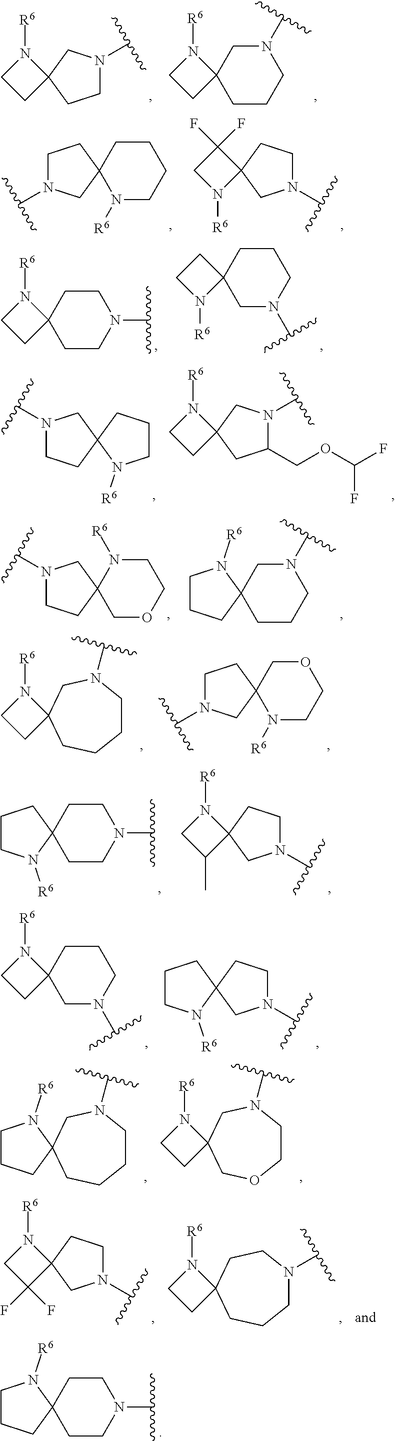

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq) (Ir), (In), (II′), or (II″), R⁷ is selected from

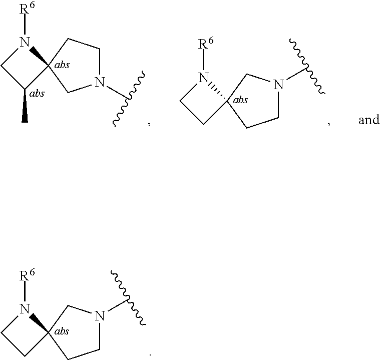

In embodiments of Formula (IV-3), (IV-4), (IV), (IVa), or (IV′), R⁷ is

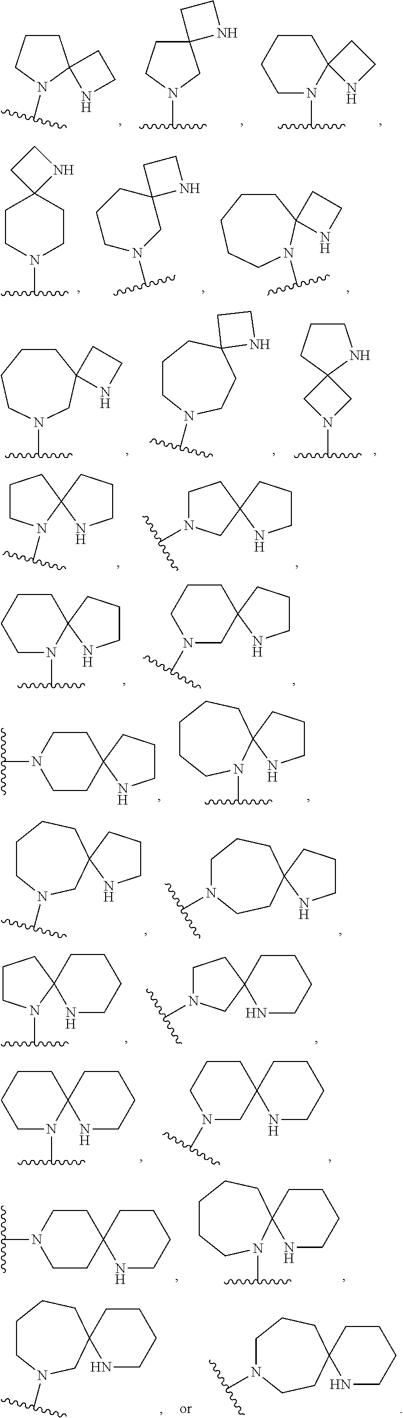

In embodiments of Formula (IV-3) (IV-4), (IV) (IVa) or (IV′), R⁷ is selected from

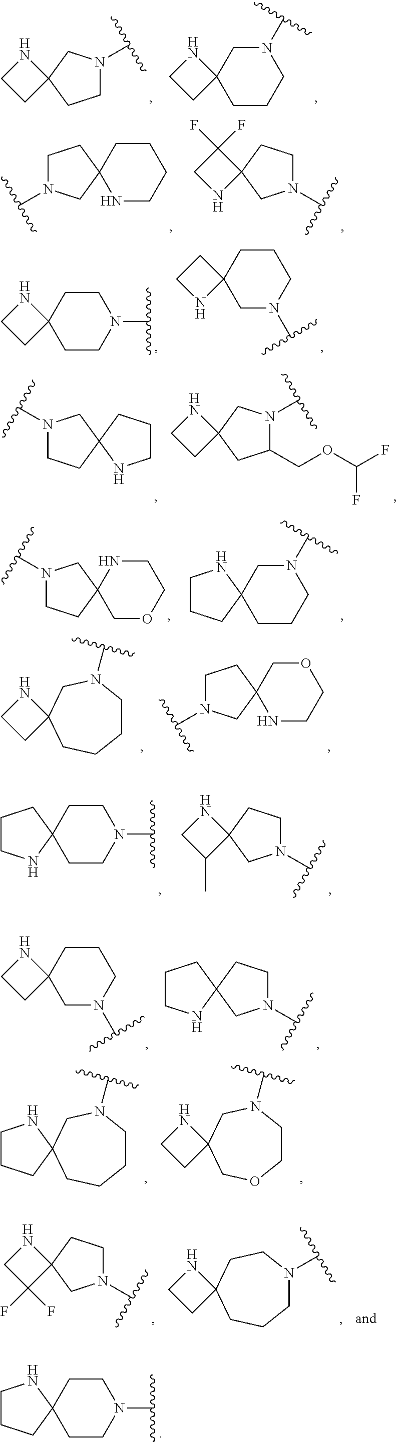

In embodiments of Formula (IV-3), (IV-4), (IV), (IVa), or (IV′), R⁷ is selected from

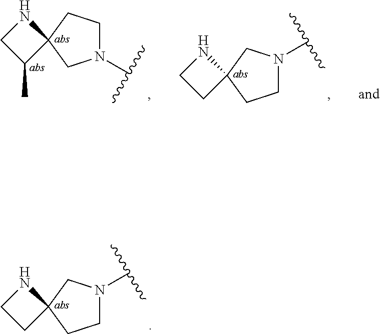

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

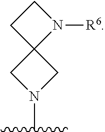

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

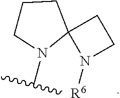

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

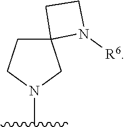

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

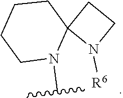

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

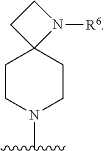

In embodiments of Formula I), I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

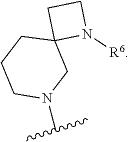

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

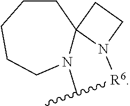

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

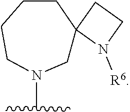

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

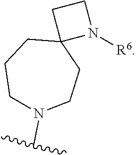

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

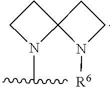

In embodiments of Formula (I), (I-3), (I-3′), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

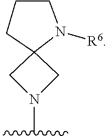

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

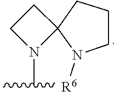

In embodiments of Formula (I), (I-3), (I-4), (II-3), (I′), (Ia), (Ib), (Ic), (Id), (Ie), (If), (Ig), (Ii), (Ij) (Ik), (Im), (In), (Io), (Ip), (Iq), (Ir), (II), (II′), or (II′″), R⁷ is

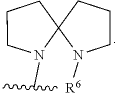

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

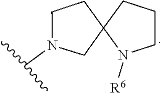

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

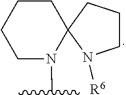

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II′″), R⁷ is

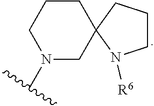

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

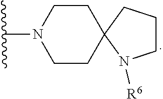

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II′″), R⁷ is

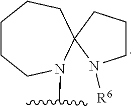

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

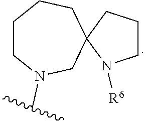

In embodiments of Formula (I), (I-3), (I-4), (II-3), (I′), embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

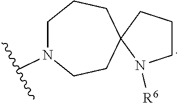

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

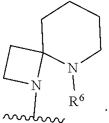

In embodiments of Formula (I), (I-3), (I-4), (II-3), (I′), embodiments of Formula (I), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

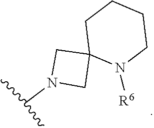

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II′″), R⁷ is

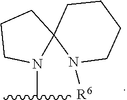

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

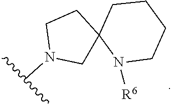

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

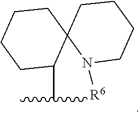

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II′″), R⁷ is

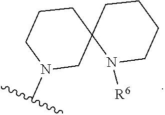

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

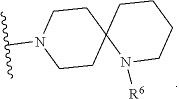

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

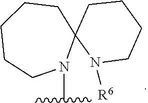

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), R⁷ is

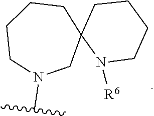

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

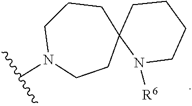

In embodiments of Formula (IV-3), (IV-4), (IV), (IVa), or (IV′), R⁷ is

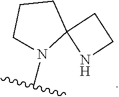 [SMILES: *N1CCCC12CCN2]

In embodiments of Formula (IV-3), (IV-4), (IV), (IVa), or (IV′), R⁷ is

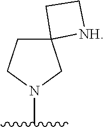 [SMILES: *N1CCC2(CCN2)C1]

In embodiments of Formula (IV-3), (IV-4), (IV), (IVa), or (IV′), R⁷ is

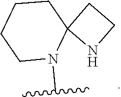 [SMILES: *N1CCCCC12CCN2]

In embodiments of Formula (IV-3), (IV-4), (IV), (IVa), or (IV′), R⁷ is

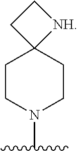 [SMILES: *N1CCC2(CCN2)CC1]

In embodiments of Formula (IV-3), (IV-4), (IV), (IVa), or (IV′), R⁷ is

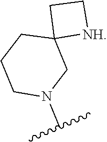 [SMILES: *N1CCCC2(CCN2)C1]

In embodiments of Formula (IV-3), (IV-4), (IV), (IVa), or (IV′), R⁷ is

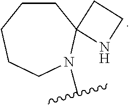 [SMILES: *N1CCCCCC12CCN2]

In embodiments of Formula (IV-3), (IV-4), (IV), (IVa), or (IV′), R⁷ is

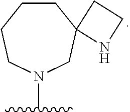 [SMILES: *N1CCCCC2(CCN2)C1]

In embodiments of Formula (IV-3), (IV-4), (IV), (IVa), or (IV′), R⁷ is

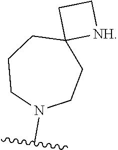 [SMILES: *N1CCCC2(CCN2)CC1]

In embodiments of Formula (IV-3), (IV-4), (IV), (IVa), or (IV′), R⁷ is

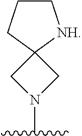 [SMILES: *N1CC2(CCCN2)C1]

In embodiments of Formula (IV-3), (IV-4), (IV), (IVa), or (IV′), R⁷ is

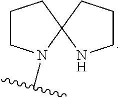 [SMILES: *N1CCCC12CCCN2]

In embodiments of Formula (IV-3), (IV-4), (IV), (IVa), or (IV′), R⁷ is

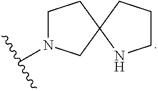 [SMILES: *N1CCC2(CCCN2)C1]

In embodiments of Formula (IV-3), (IV-4), (IV), (IVa), or (IV′), R⁷ is

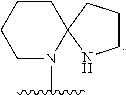 [SMILES: *N1CCCCC12CCCN2]

In embodiments of Formula (IV-3), (IV-4), (IV), (IVa), or (IV′), R⁷ is

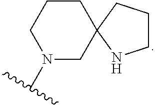 [SMILES: *N1CCCC2(CCCN2)C1]

In embodiments of Formula (IV-3), (IV-4), (IV), (IVa), or (IV′), R⁷ is

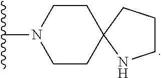 [SMILES: *N1CCC2(CCCN2)CC1]

In embodiments of Formula (IV-3), (IV-4), (IV), (IVa), or (IV′), R⁷ is

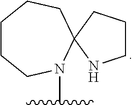 [SMILES: *N1CCCCCC12CCCN2]

In embodiments of Formula (IV-3), (IV-4), (IV), (IVa), or (IV′), R⁷ is

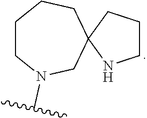 [SMILES: *N1CCCCC2(CCCN2)C1]

In embodiments of Formula (IV-3), (IV-4), (IV), (IVa), or (IV′), R⁷ is

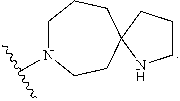 [SMILES: *N1CCCC2(CCCN2)CC1]

In embodiments of Formula (IV-3), (IV-4), (IV), (IVa), or (IV′), R⁷ is

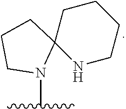 [SMILES: *N1CCCC12CCCCN2]

In embodiments of Formula (IV-3), (IV-4), (IV), (IVa), or (IV′), R⁷ is

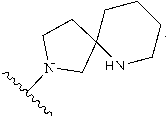 [SMILES: *N1CCC2(CCCCN2)C1]

In embodiments of Formula (IV-3), (IV-4), (IV), (IVa), or (IV′), R⁷ is

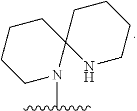 [SMILES: *N1CCCCC12CCCCN2]

In embodiments of Formula (IV-3), (IV-4), (IV), (IVa), or (IV′), R⁷ is

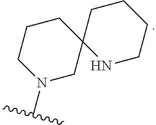 [SMILES: *N1CCCC2(CCCCN2)C1]

In embodiments of Formula (IV-3), (IV-4), (IV), (IVa), or (IV′), R⁷ is

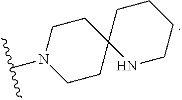 [SMILES: *N1CCC2(CCCCN2)CC1]

In embodiments of Formula (IV-3), (IV-4), (IV), (IVa), or (IV′), R⁷ is

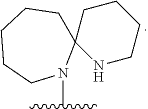 [SMILES: *N1CCCCCC12CCCCN2]

In embodiments of Formula (IV-3), (IV-4), (IV), (IVa), or (IV′), R⁷ is

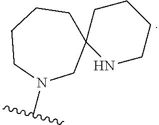 [SMILES: *N1CCCCC2(CCCCN2)C1]

In embodiments of Formula (IV-3), (IV-4), (IV), (IVa), or (IV′), R⁷ is

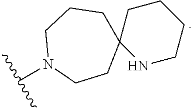 [SMILES: *N1CCCC2(CCCCN2)CC1]

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

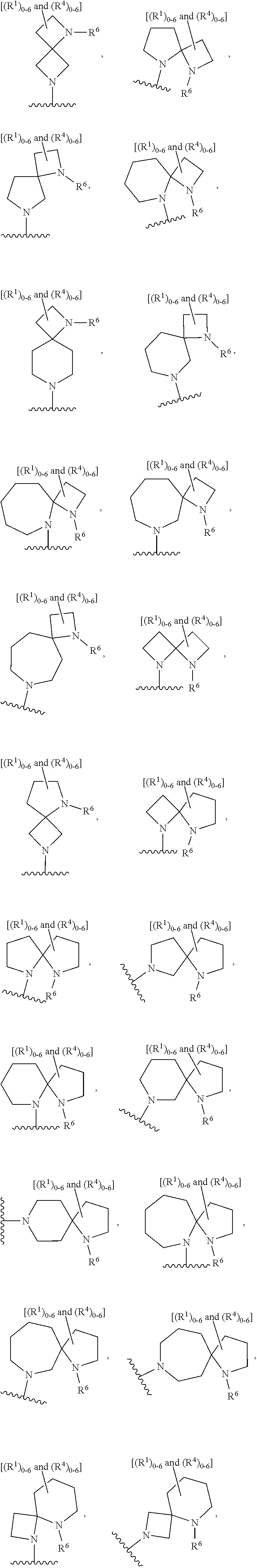

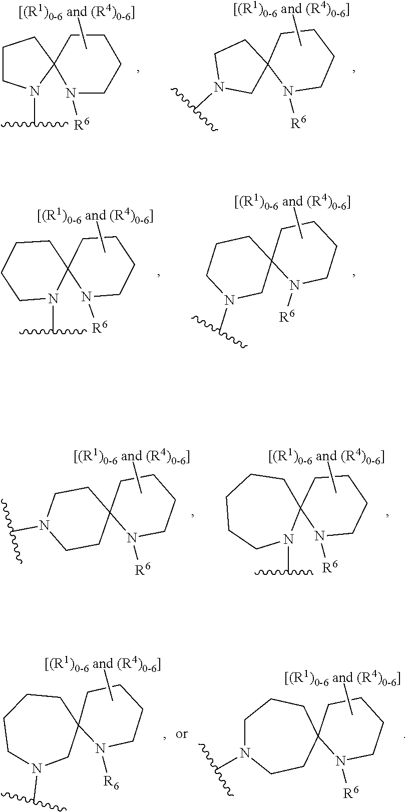

In embodiments of Formula (IV-3), (IV-4), (IV), (IVa), or (IV′), R⁷ is

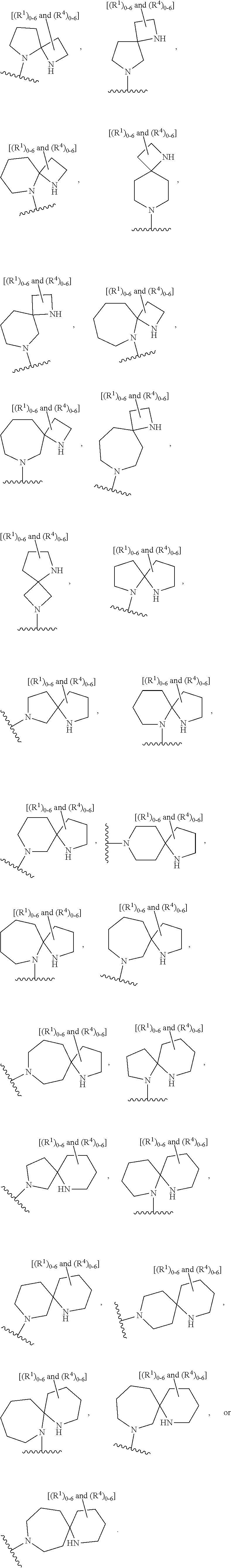

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is

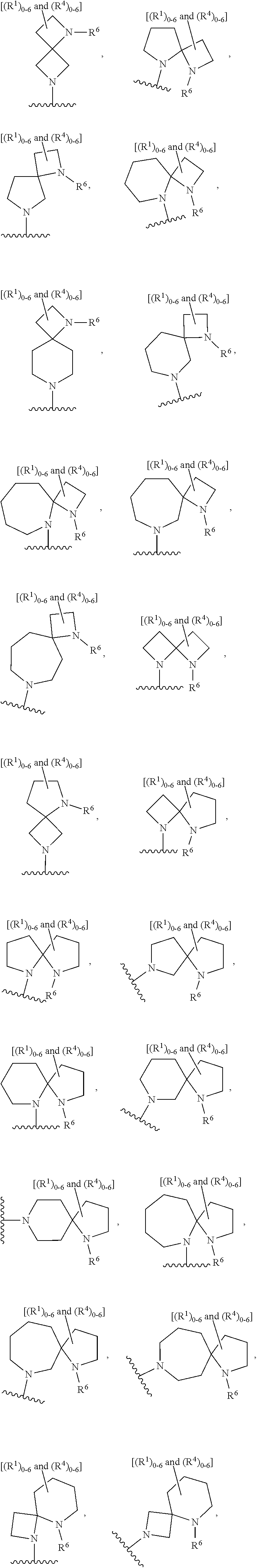

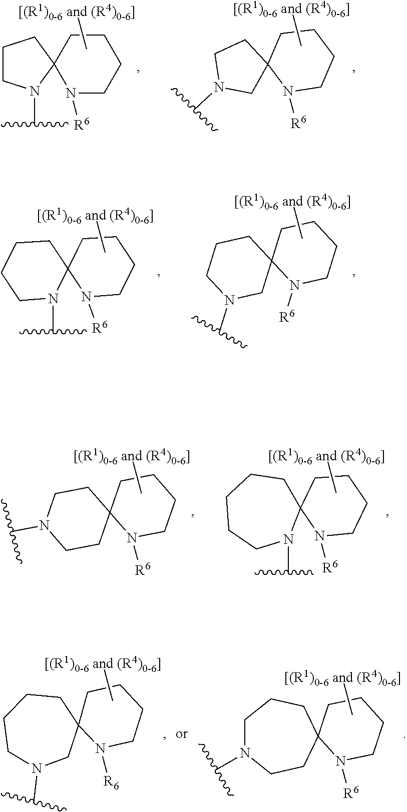

In embodiments of Formula (IV-3), (IV-4), (IV), (IVa), or (IV′), R⁷ is

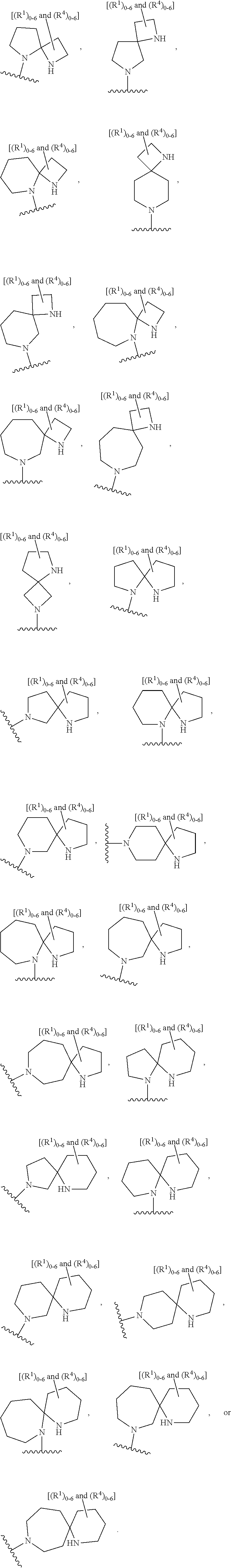

It will be understood that when one or more floating substituent(s) is/are shown extending from one ring in a polycyclic ring system (e.g., fused ring system, bridged ring system, or spirocyclic ring system), the one or more floating substituent(s), may be bonded to the ring from which the one or more floating substituents are shown extending or may be bonded to any other ring in the polycyclic ring system (e.g., fused ring system, spirocyclic ring system, or bridged ring system) and when multiple substituents are represented by the floating substituents, each substituent may be bonded to the same or different rings in the polycyclic ring system, unless indicated otherwise.

In an embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is selected from

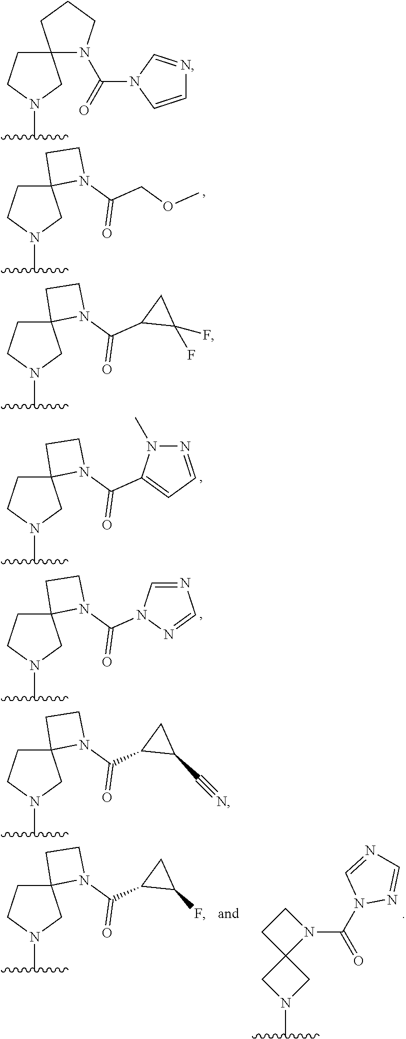

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is selected from

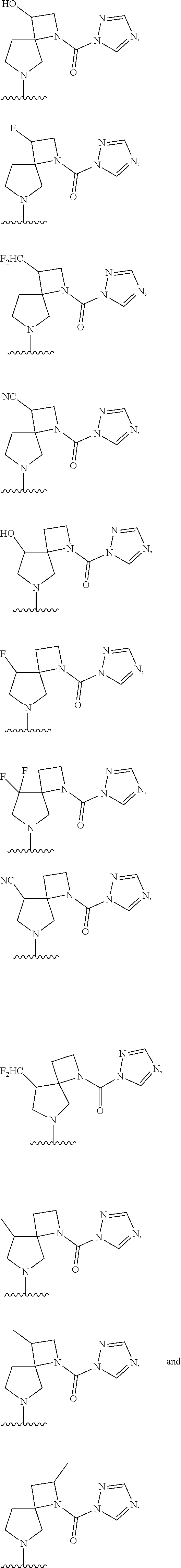

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R¹⁰ is selected from

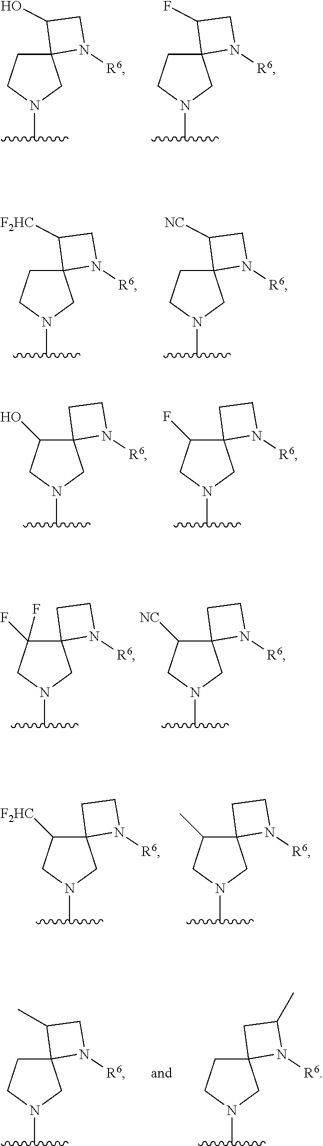

In an embodiments of Formula (I), (I-3), (I-4), (II-3), (I′), (Ia), (Ib), (Ic), (Id), (e), (If), (Ig), (Ii), (Ij), (Ik), (Im), (In), (Io), (Ip), (Iq), (Ir), (II), (II′), or (II″), R⁷ is selected from

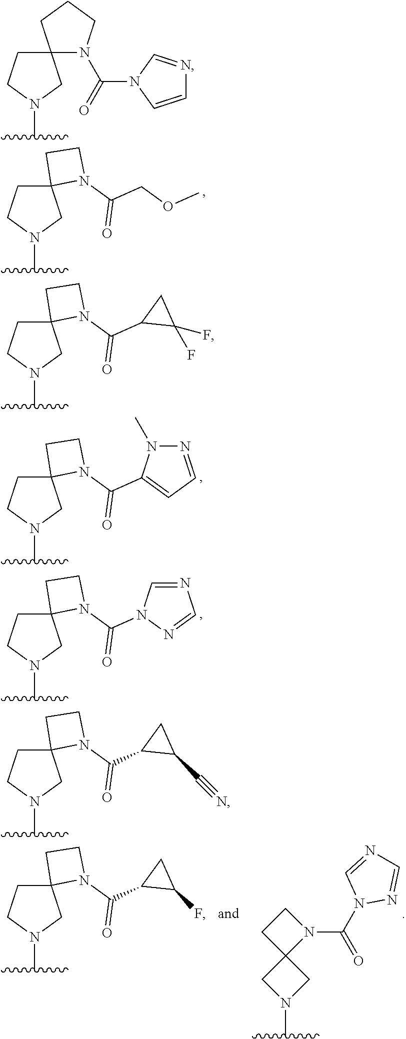

In embodiments of Formula (I), (I-3), (I-4), (II-3), (I′), (Ia), (Ib) (Ic), (Id), (Ie), (If), (Ig), (Ii), (Ij), (Ik), (Im), (In), (Io), (Ip), (Ig), (Ir), (II), (II′), or (II″), R⁷ is selected from

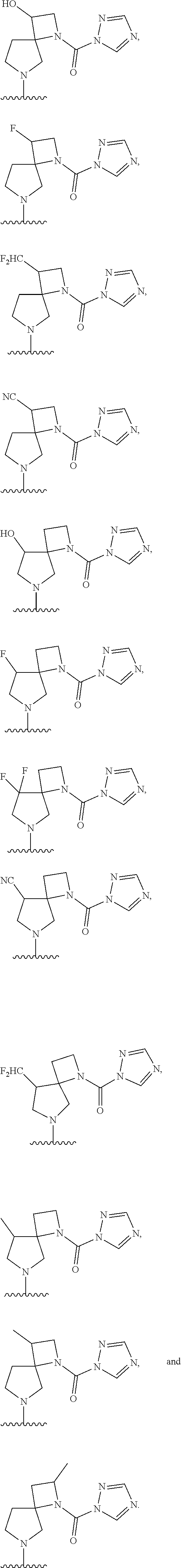

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

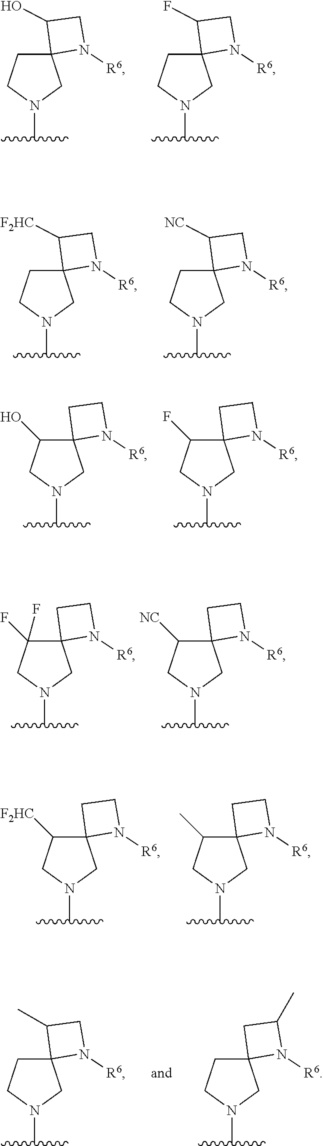

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

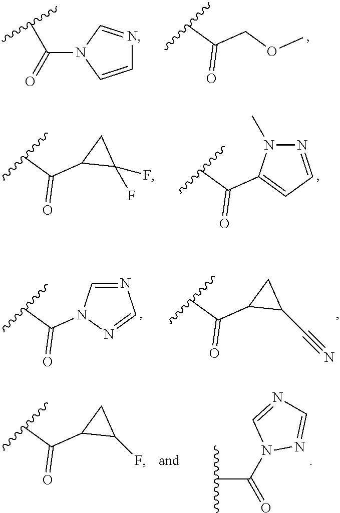

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

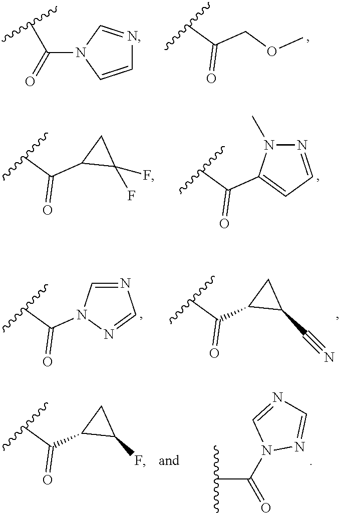

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

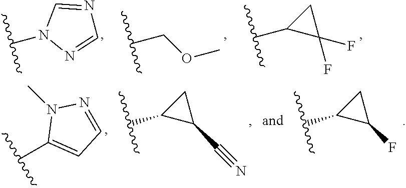

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

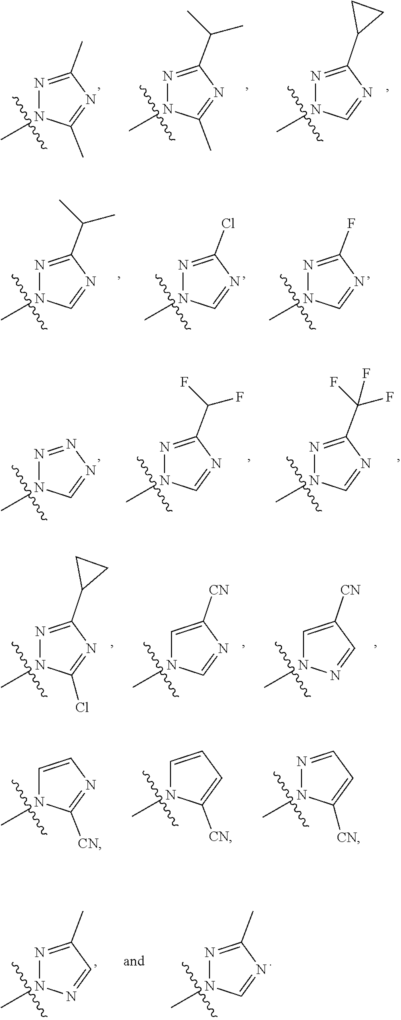

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

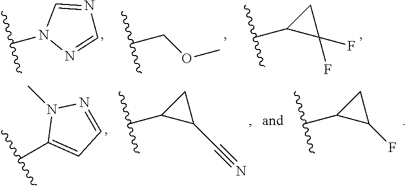

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

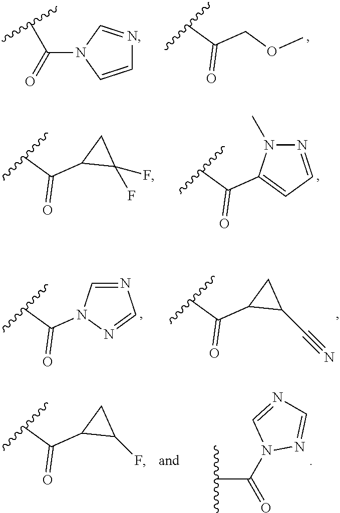

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁶ is selected from

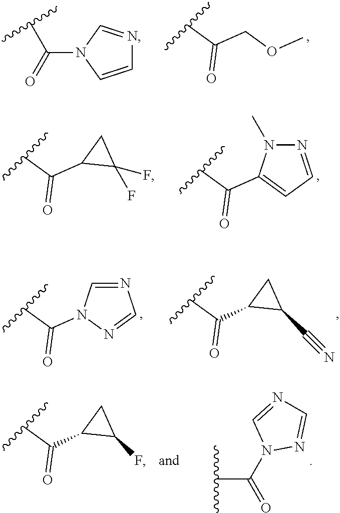

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is selected from

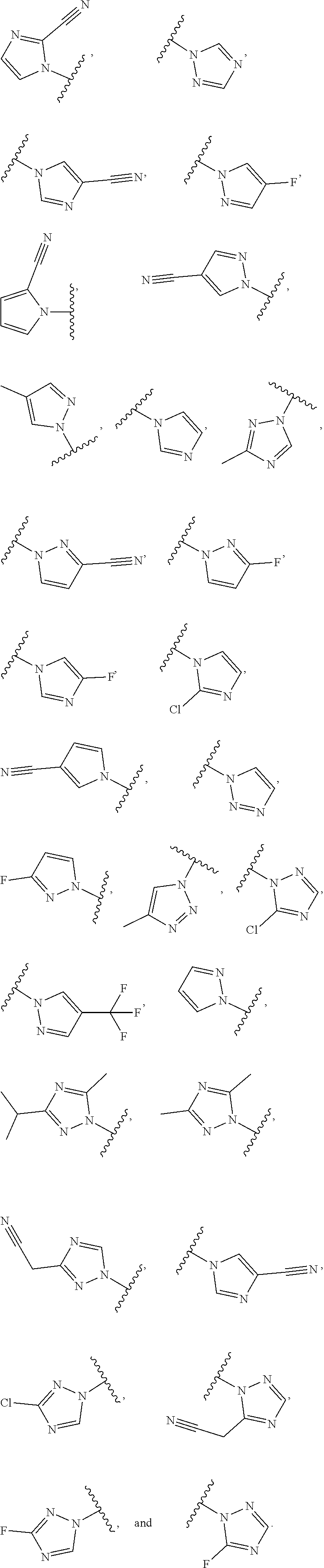

In embodiments of Formula I, (I-3), (I-4), (II-3), (I′), (Ia), (Ib), (Ic), (Id), (Ii), (Ij) (Ik), (Im), (In), (Io), (Ip), (Iq), (Ir), (II), (II′), or (II″), R⁵ is selected from

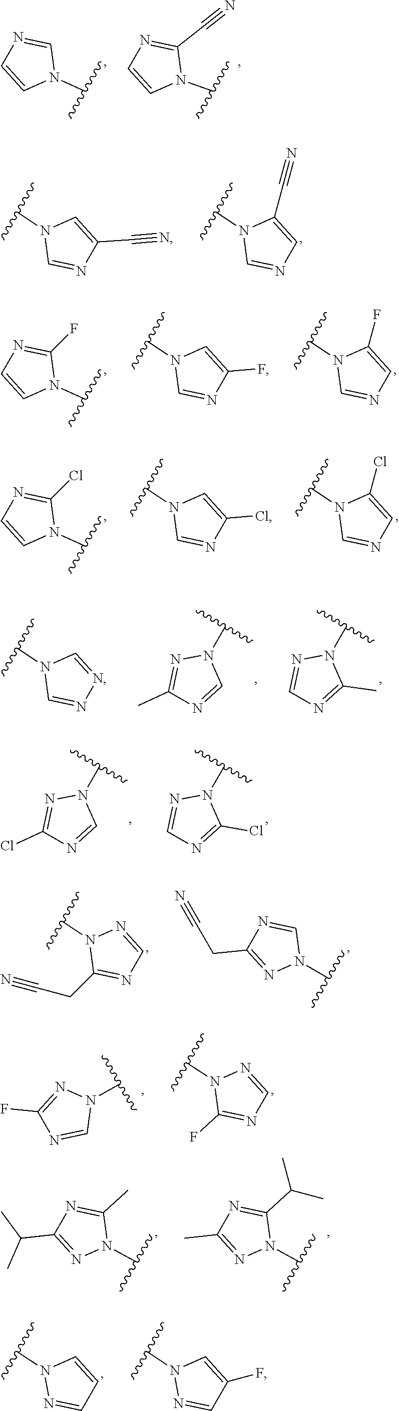

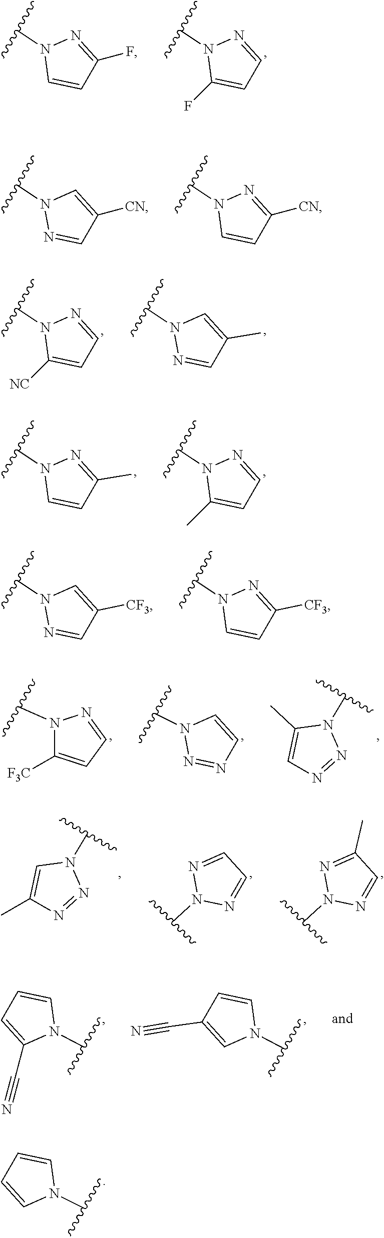

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II′″), R⁵ is selected from

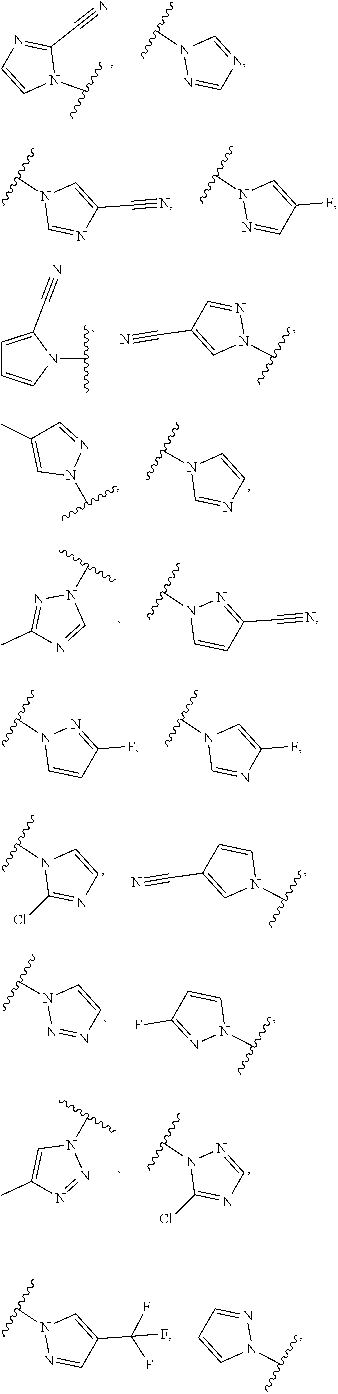

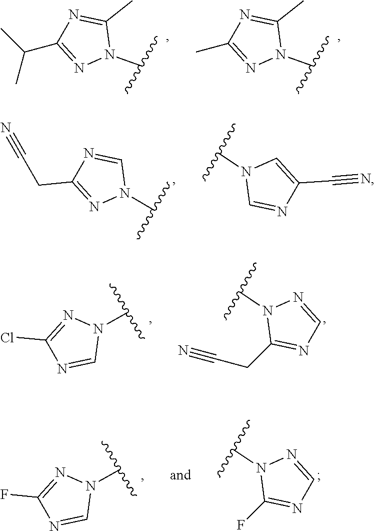

each of which is optionally substituted with one or more R^{20k}. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II′″), R⁵ is selected from

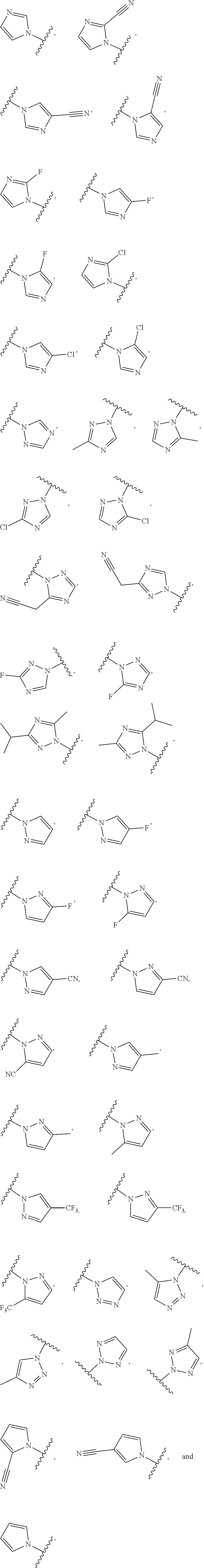

each of which is optionally substituted with one or more R^{20k}. In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″), R⁵ is selected from

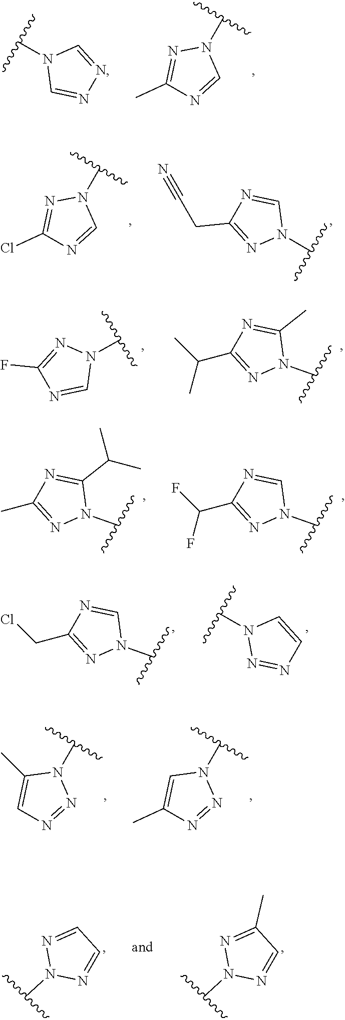

each of which is optionally substituted with one or more R^{20k}.

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is selected from a —C(O)—, —N(R^{4d})C(O)—, —C(O)N(R^{4d})—, and —CH₂N(R^{4d})C(O)—. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is selected from a bond, —C(O)NH—, —NHC(O)—, and —C(O)—.

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁵ is a 5-6 membered heteroaryl optionally substituted with one, two, three, or four R^{20k}, wherein the heteroaryl comprises one, two, three, or four, ring nitrogen atoms and further wherein when R⁵ is directly bonded to a C(O), S(O), or S(O)₂ of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁵ is a 5 membered heteroaryl optionally substituted with one, two, three, or four R^{20k}, wherein the heteroaryl comprises one, two, or three ring nitrogen atoms and further wherein when R⁵ is directly bonded to a C(O), S(O), or S(O)₂ of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁵ is a pyrrolyl, pyrazolyl, imidazolyl, or triazolyl optionally substituted with one, two, three, or four R^{20k}, wherein when R⁵ is directly bonded to a C(O), S(O), or S(O)₂ of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁵ is a triazolyl optionally substituted with one, two, three, or four R^{20k}, wherein when R⁵ is directly bonded to a C(O), S(O), or S(O)₂ of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁵ is a pyrrolyl, pyrazolyl, imidazolyl, oxazolyl, isoxazolyl, thiazolyl, isothiazolyl, pyridyl, pyridazinyl, pyrimidinyl, pyrazinyl, triazinyl, oxadiazolyl, thiadiazolyl, or triazolyl optionally substituted with one, two, three, or four R^{20k}, wherein when R⁵ is directly bonded to a C(O), S(O), or S(O)₂ of L², L² is directly bonded to an N atom of R⁵.

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is a —C(O)—, —N(R^{4d})C(O)—, or —C(O)N(R^{4d})—; and R⁵ is a 5-6 membered heteroaryl optionally substituted with one, two, three, or four R^{20k}, wherein the heteroaryl comprises one, two, three, or four, ring nitrogen atoms and further wherein when R⁵ is directly bonded to a C(O) of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is a —C(O)—, —N(R^{4d})C(O)—, or —C(O)N(R^{4d})—; and R⁵ is a 5 membered heteroaryl optionally substituted with one, two, three, or four R^{20k}, wherein the heteroaryl comprises one, two, or three ring nitrogen atoms and further wherein when R⁵ is directly bonded to a C(O) of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is a —C(O)—, —N(R^{4d})C(O)—, or —C(O)N(R^{4d})—; and R⁵ is a pyrrolyl, pyrazolyl, imidazolyl, or triazolyl optionally substituted with one, two, three, or four R^{20k}, wherein when R⁵ is directly bonded to a C(O) of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is a —C(O)—, —N(R^{4d})C(O)—, or —C(O)N(R^{4d})—; and R⁵ is a triazolyl optionally substituted with one, two, three, or four R^{20k}, wherein when R⁵ is directly bonded to a C(O) of L², L² is directly bonded to an N atom of R⁵. In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), L² is a —C(O)—, —N(R^{4d})C(O)—, or —C(O)N(R^{4d})—; and R⁵ is a pyrrolyl, pyrazolyl, imidazolyl, oxazolyl, isoxazolyl, thiazolyl, isothiazolyl, pyridyl, pyridazinyl, pyrimidinyl, pyrazinyl, triazinyl, oxadiazolyl, thiadiazolyl, or triazolyl optionally substituted with one, two, three, or four R^{20k}, wherein when R⁵ is directly bonded to a C(O) of L², L² is directly bonded to an N atom of R⁵.

The individual embodiments herein below, or combinations thereof, (e.g., embodiments of R⁵ or R⁶) are applicable to compounds of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), or a pharmaceutically acceptable salt or solvate thereof. In embodiments of the formulae above, R⁵ is C₂₋₆alkenyl optionally substituted with one, two, or three R^{20k}. In embodiments of the formulae above, R⁵ is C₂₋₆alkenyl optionally substituted with one, two, or three halogen. In embodiments of the formulae above, R⁵ is C₂₋₆alkenyl optionally substituted with one, two, or three F. In embodiments of the formulae above, R⁵ is C₂₋₆alkenyl optionally substituted with —OR²¹. In embodiments of the formulae above, R⁵ is C₂₋₆alkenyl optionally substituted with —N(R²²)(R²³). In embodiments of the formulae above, R⁵ is C₂₋₆alkenyl optionally substituted with C₂₋₉heterocycloalkyl. In embodiments of the formulae above, R⁵ is C₂₋₆alkenyl optionally substituted with C₁₋₅ heteroaryl optionally substituted with one, two, or three C₁₋₆alkyl. In embodiments of the formulae above, R⁵ is C₂₋₆alkenyl optionally substituted with C₁₋₅heteroaryl optionally substituted with methyl. In embodiments of the formulae above, R⁵ is C₂₋₆alkenyl optionally substituted with —CN. In embodiments of the formulae above, R⁵ is C₂₋₆alkenyl optionally substituted with C₁₋₅heteroaryl optionally substituted with —N(R²⁴)C(O)R²⁵. In embodiments of the formulae above, R⁵ is C₂₋₆alkenyl optionally substituted with C₁₋₅ heteroaryl and/or CN. In embodiments of the formulae above, R⁵ is C₂₋₆alkenyl optionally substituted with one, two, or three R^{20k}. In embodiments of the formulae above, R⁵ is C₂₋₆alkenyl optionally substituted with one, two, or three halogen. In embodiments of the formulae above, R⁵ is C₂₋₆alkenyl optionally substituted with one, two, or three F. In embodiments of the formulae above, R⁵ is C₂₋₆alkenyl optionally substituted with —OR²¹. In embodiments of the formulae above, R⁵ is C₂₋₆alkenyl optionally substituted with —N(R²²)(R²³). In embodiments of the formulae above, R⁵ is C₂₋₆alkenyl optionally substituted with C₂₋₉heterocycloalkyl. In embodiments of the formulae above, R⁵ is C₂₋₆alkenyl optionally substituted with C₁₋₅heteroaryl optionally substituted with one, two, or three C₁₋₆alkyl. In embodiments of the formulae above, R⁵ is C₂₋₆alkenyl optionally substituted with C₁₋₅heteroaryl optionally substituted with methyl. In embodiments of the formulae above, R⁵ is C₂₋₆alkenyl optionally substituted with —CN. In embodiments of the formulae above, R⁵ is C₂₋₆alkenyl optionally substituted with C₁₋₅heteroaryl optionally substituted with —N(R²⁴)C(O)R²⁵. In embodiments of the formulae above, R⁵ is C₂₋₆alkenyl optionally substituted with —C(O)N(R²²)(R²³). In embodiments of the formulae above, R⁵ is C₂₋₆alkenyl substituted with Cl and optionally substituted with one or two R^{20k}. In embodiments of the formulae above, R⁵ is C₂₋₆alkynyl optionally substituted with one, two, or three R^{20k}. In embodiments of the formulae above, R⁵ is C₂₋₆alkynyl optionally substituted with one, two, or three halogen. In embodiments of the formulae above, R⁵ is C₂₋₆alkynyl optionally substituted with one, two, or three F. In embodiments of the formulae above, R⁵ is C₂₋₆alkynyl optionally substituted with —OR²¹. In embodiments of the formulae above, R⁵ is C₂₋₆alkynyl optionally substituted with —N(R²²)(R²³). In embodiments of the formulae above, R⁵ is C₂₋₆alkynyl optionally substituted with C₂₋₉heterocycloalkyl. In embodiments of the formulae above, R⁵ is C₂₋₆alkynyl optionally substituted with C₁₋₅heteroaryl optionally substituted with one, two, or three C₁₋₆alkyl. In embodiments of the formulae above, R⁵ is C₂₋₆alkynyl optionally substituted with C₁₋₅heteroaryl optionally substituted with methyl. In embodiments of the formulae above, R⁵ is C₂₋₆alkynyl optionally substituted with —CN. In embodiments of the formulae above, R⁵ is C₂₋₆alkynyl optionally substituted with C₁₋₅heteroaryl optionally substituted with —N(R²⁴)C(O)R²⁵. In embodiments of the formulae above, R⁵ is C₂₋₆alkynyl optionally substituted with C₁₋₅heteroaryl and/or CN. In embodiments of the formulae above, R⁵ is C₂₋₆alkynyl optionally substituted with one, two, or three R^{2I}k. In embodiments of the formulae above, R⁵ is C₂₋₆alkynyl optionally substituted with one, two, or three halogen. In embodiments of the formulae above, R⁵ is C₂₋₆alkynyl optionally substituted with one, two, or three F. In embodiments of the formulae above, R⁵ is C₂₋₆alkynyl optionally substituted with —OR²¹. In embodiments of the formulae above, R⁵ is C₂₋₆alkynyl optionally substituted with —N(R²²)(R²³). In embodiments of the formulae above, R⁵ is C₂₋₆alkynyl optionally substituted with C₂₋₉heterocycloalkyl. In embodiments of the formulae above, R⁵ is C₂₋₆alkynyl optionally substituted with C₁₋₅heteroaryl optionally substituted with one, two, or three C₁₋₆alkyl. In embodiments of the formulae above, R⁵ is C₂₋₆alkynyl optionally substituted with C₁₋₅ heteroaryl optionally substituted with methyl. In embodiments of the formulae above, R⁵ is C₂₋₆alkynyl optionally substituted with —CN. In embodiments of the formulae above, R⁵ is C₂₋₆alkynyl optionally substituted with C₁₋₅heteroaryl optionally substituted with —N(R²⁴)C(O)R²⁵. In embodiments of the formulae above, R⁵ is C₂₋₆alkynyl optionally substituted with —C(O)N(R²²)(R²³). In embodiments of the formulae above, R⁵ is C₂₋₆alkynyl substituted with Cl and optionally substituted with one or two R^{20k}. In embodiments of the formulae above, R⁵ is C₃₋₅cycloalkyl, optionally substituted with one, two, or three Wok. In embodiments of the formulae above, R⁵ is C₃₋₅cycloalkyl, optionally substituted with C₁₋₆alkyl optionally substituted with one, two, or three halogen. In embodiments of the formulae above, R⁵ is C₃₋₅cycloalkyl, optionally substituted with C₁₋₆alkyl optionally substituted with one, two, or three F. In embodiments of the formulae above, R⁵ is C₁₋₅heterocycloalkyl, optionally substituted with one, two, or three R^{2k}. In embodiments of the formulae above, R⁵ is C₁₋₅heterocycloalkyl, optionally substituted with C₁₋₆alkyl optionally substituted with one, two, or three halogen. In embodiments of the formulae above, R⁵ is C₁₋₅heterocycloalkyl, optionally substituted with C₁₋₆alkyl optionally substituted with one, two, or three F. In embodiments of the formulae above, R⁵ is C₁₋₆alkyl, optionally substituted with one, two, or three R^{20k}. In embodiments of the formulae above, R⁵ is C₁₋₆alkyl, substituted with Cl and optionally substituted with one, two, or three R^{2k}. In embodiments of the formulae above, R⁵ is —S(O)₂R¹⁵. In embodiments of the formulae above, R⁵ is —CN.

In embodiments of the formulae above, R⁶ is selected from

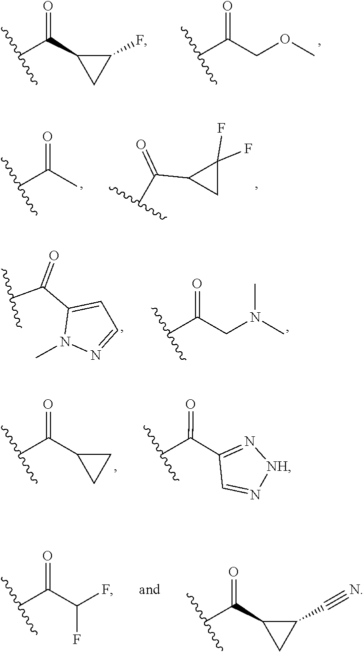

In embodiments of the formulae above, R⁶ is selected from

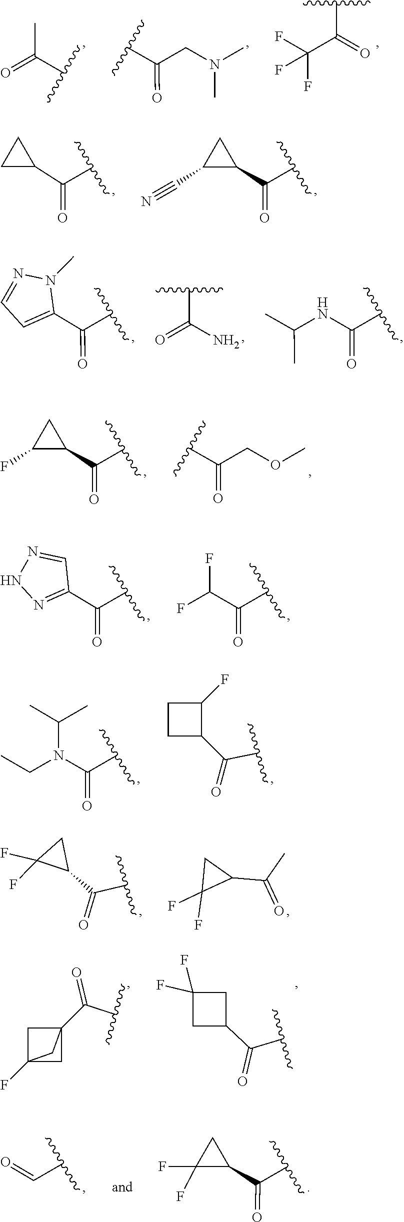

In embodiments of the formulae above, R⁶ is selected from

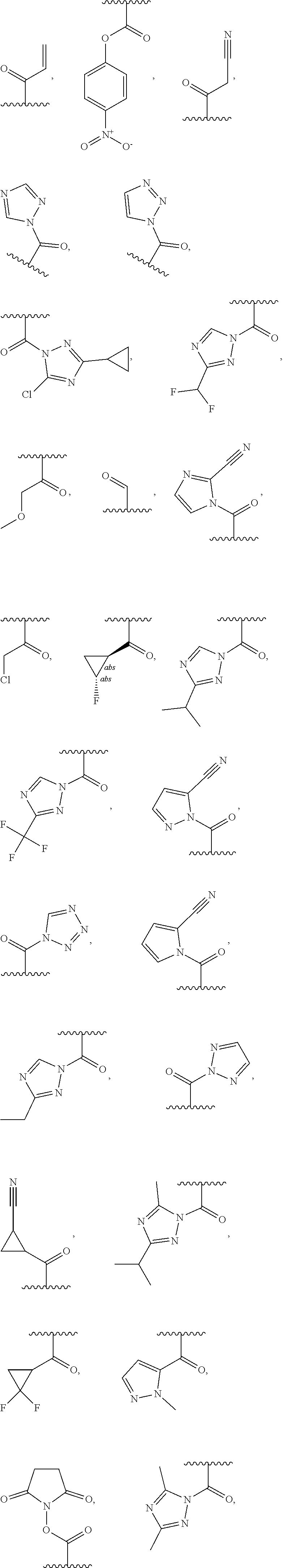

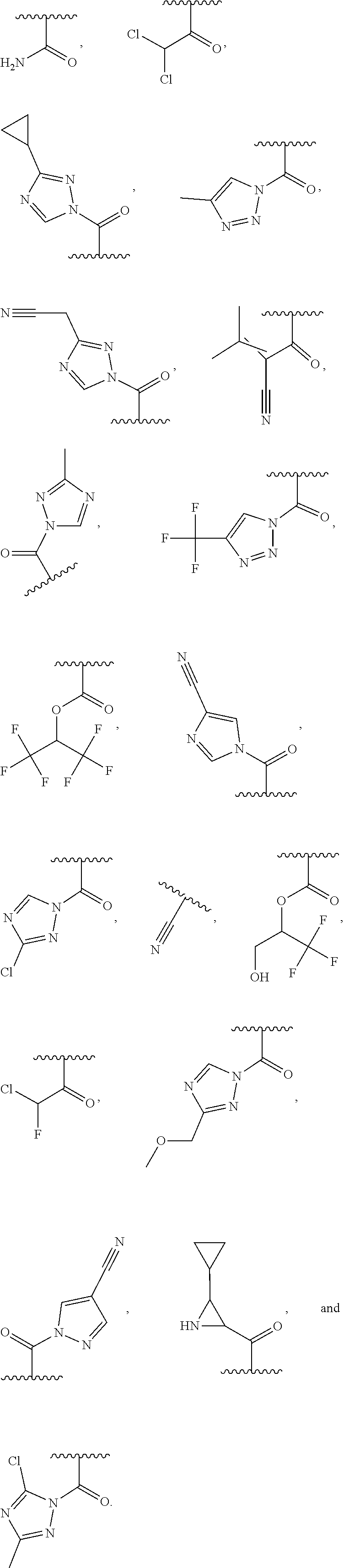

In embodiments of the formulae above, R⁶ is selected from

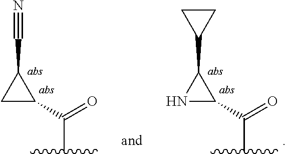 [SMILES: *C(=O)[C@@H]1C[C@H]1C#N ; *C(=O)[C@@H]1N[C@H]1C1CC1]

In embodiments of the formulae above, R⁵ is C₁₋₆alkyl optionally substituted with one, two, or three R^{20k}. In embodiments of the formulae above, R⁵ is C₁₋₆alkyl optionally substituted with —N(R²²)(R²³). In embodiments of the formulae above, R⁵ is C₁₋₆alkyl optionally substituted with one, two, or three halogen. In embodiments of the formulae above, R⁵ is C₁₋₆alkyl optionally substituted with one, two, or three F. In embodiments of the formulae above, R⁵ is C₁₋₆alkyl optionally substituted with —OR²¹. In embodiments of the formulae above, R⁵ is C₃₋₆cycloalkyl optionally substituted with one, two, or three R^{20k}. In embodiments of the formulae above, R⁵ is C₃₋₆cycloalkyl optionally substituted with —CN. In embodiments of the formulae above, R⁵ is C₃₋₆cycloalkyl optionally substituted with one, two, or three halogen. In embodiments of the formulae above, R⁵ is C₃₋₆cycloalkyl optionally substituted with one, two, or three F. In embodiments of the formulae above, R⁵ is C₁₋₅heteroaryl optionally substituted with one, two, or three R^{20k}. In embodiments of the formulae above, R⁵ is C₁₋₅heteroaryl optionally substituted with methyl. In embodiments of the formulae above, R⁵ is C₁₋₅heteroaryl optionally substituted with C₁₋₆alkyl.

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), or (II″)ᵣ, R⁷ is selected from

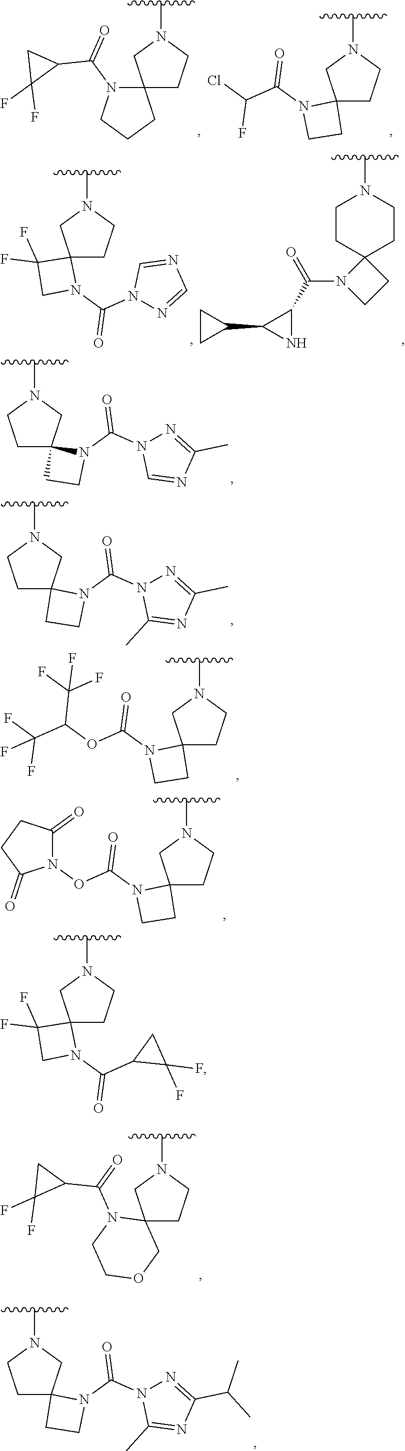

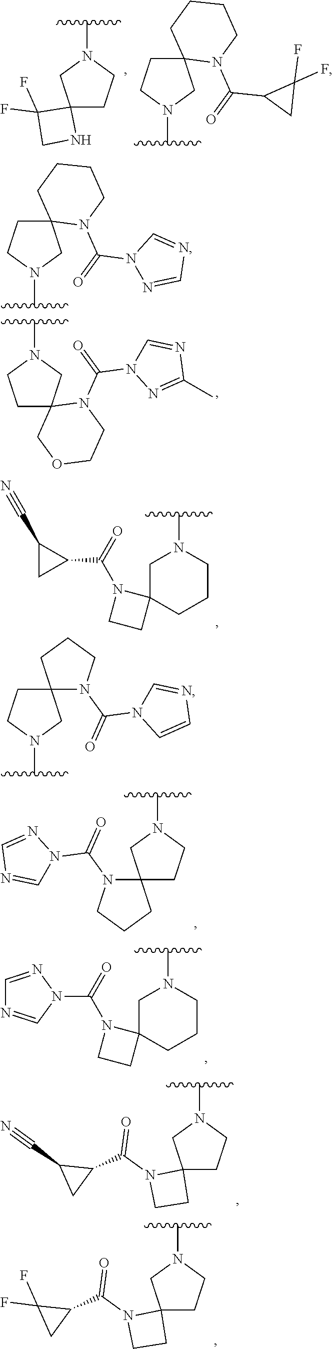

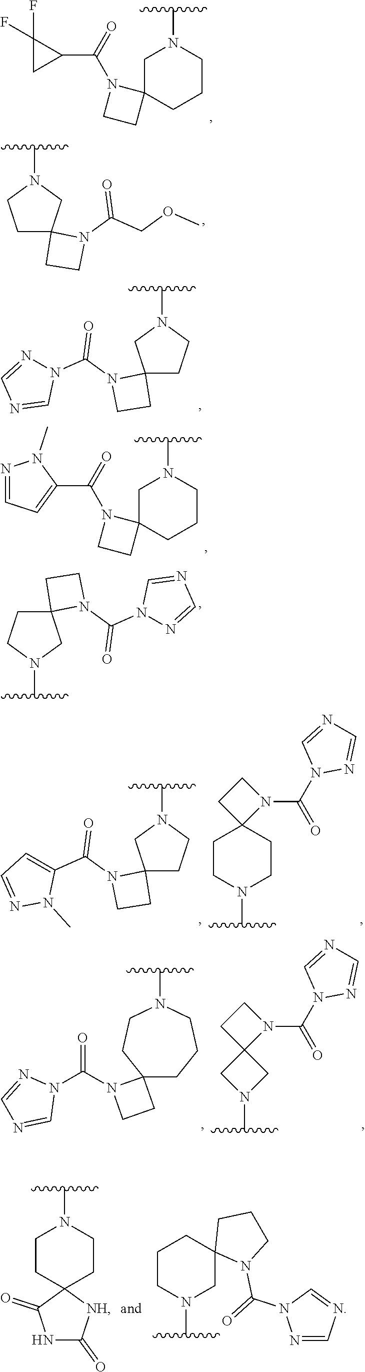

In embodiments of Formula (I), (I-3)₉ (I-4), (II-3), (I′), (Ia), (Ib), (Ic), (Id), (Ii), (Ij), (Ik), (Im), (In), (Io), (Ip), (Iq), (Ir), (II), (II′), (II″), (IV-3), (IV-4), (IV), (IVa), or (IV′), R⁷ is selected from

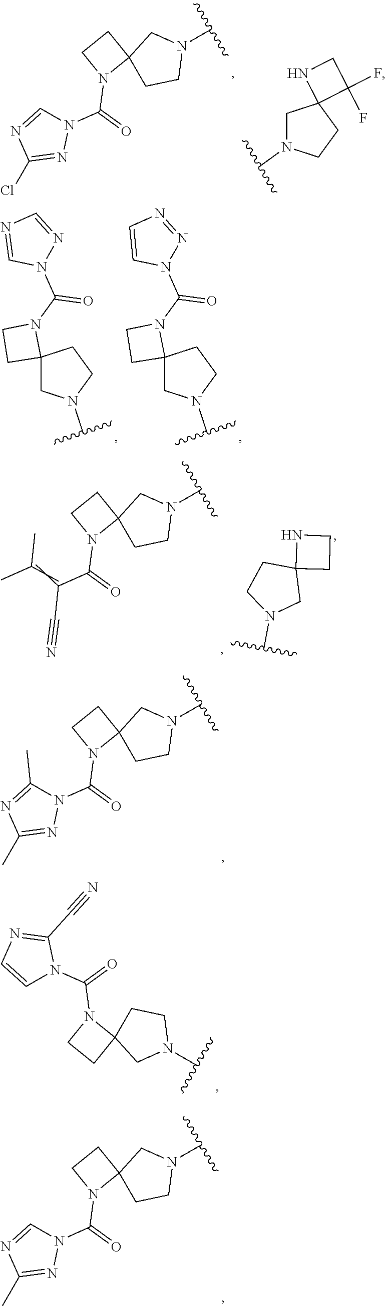

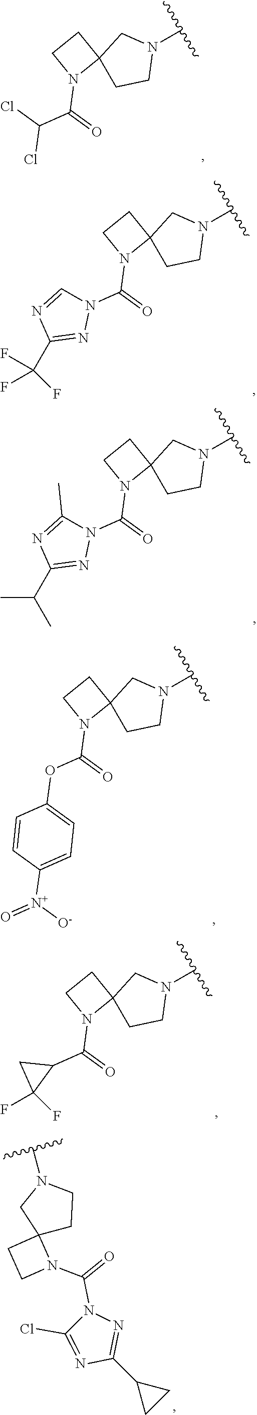

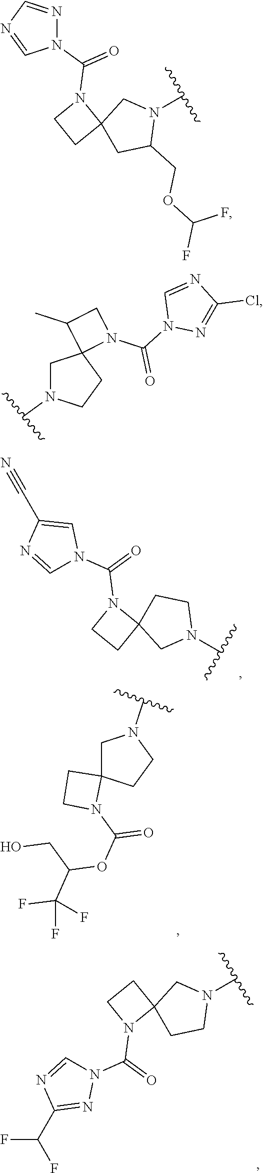

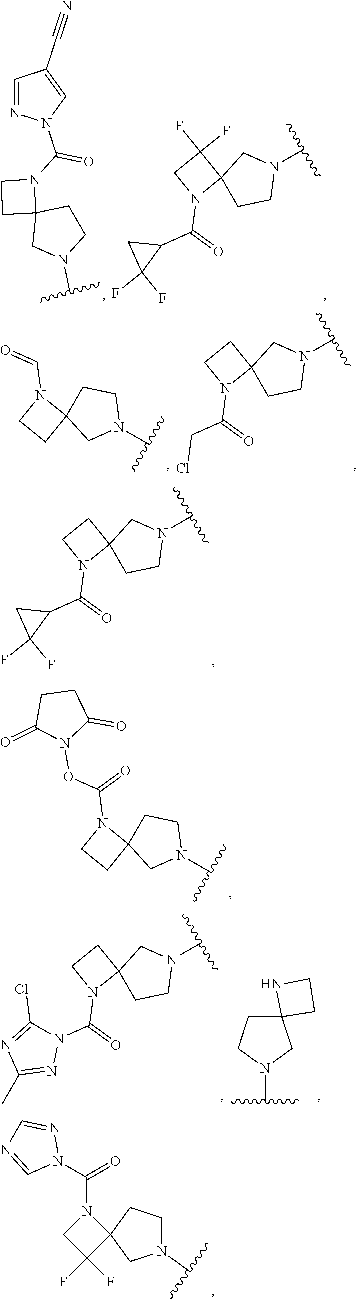

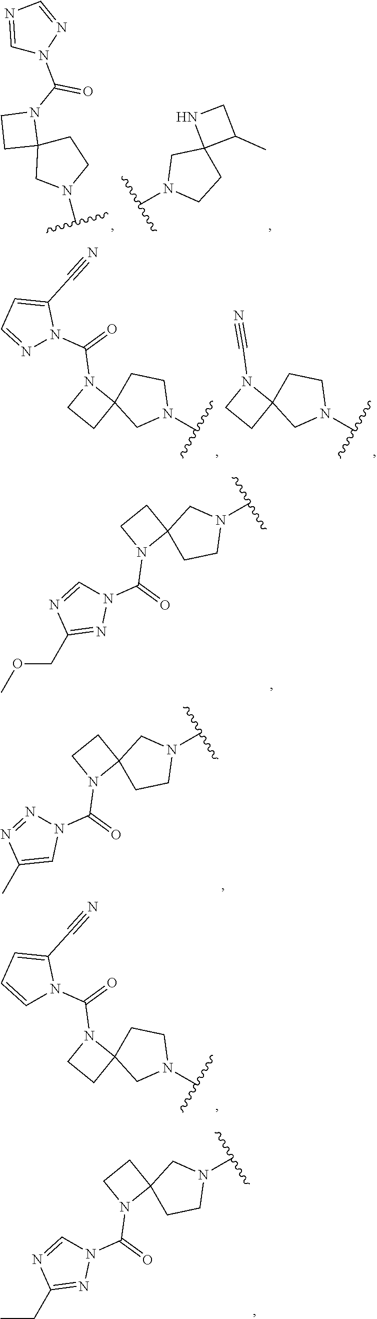

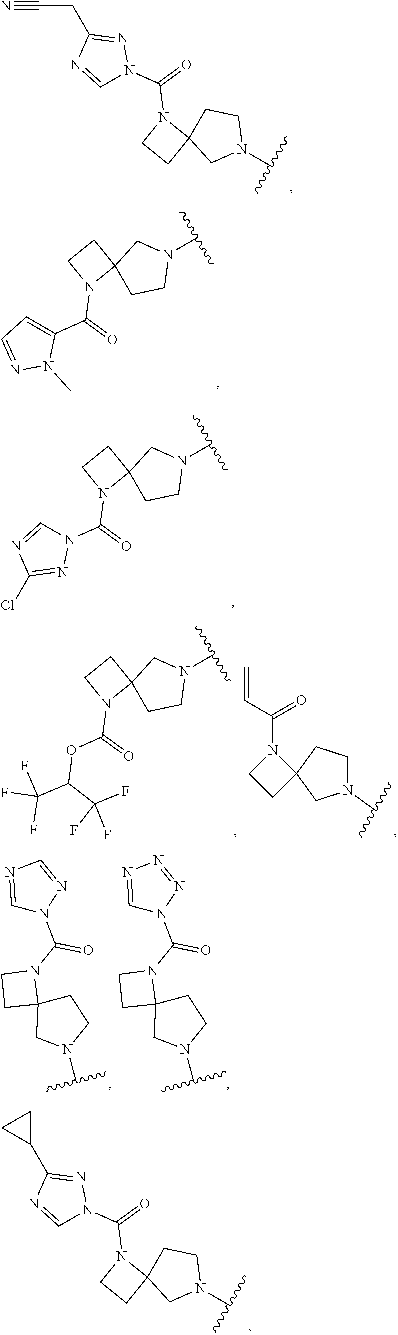

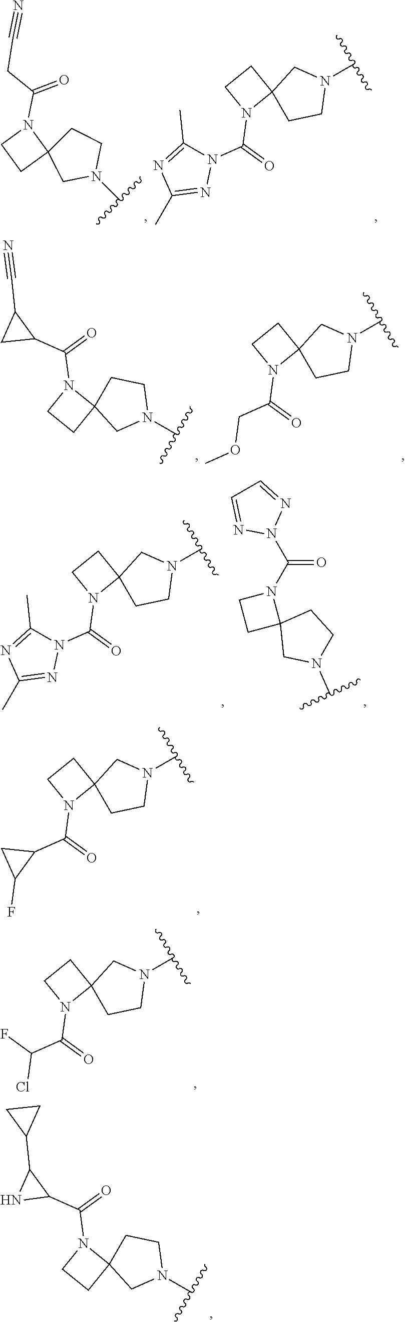

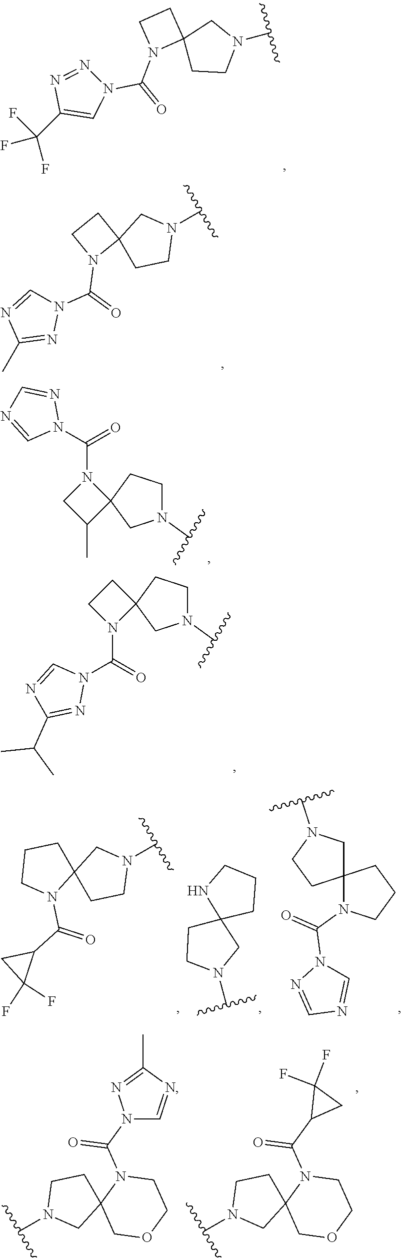

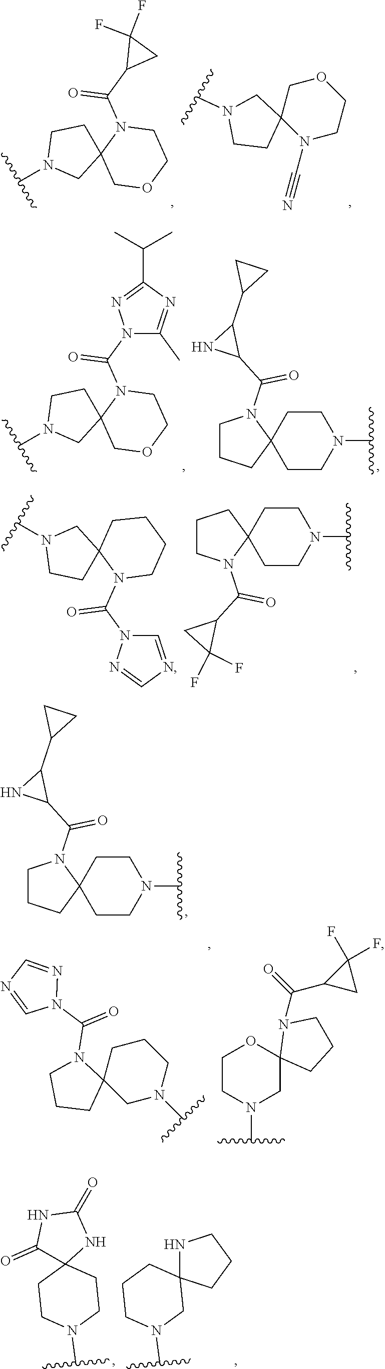

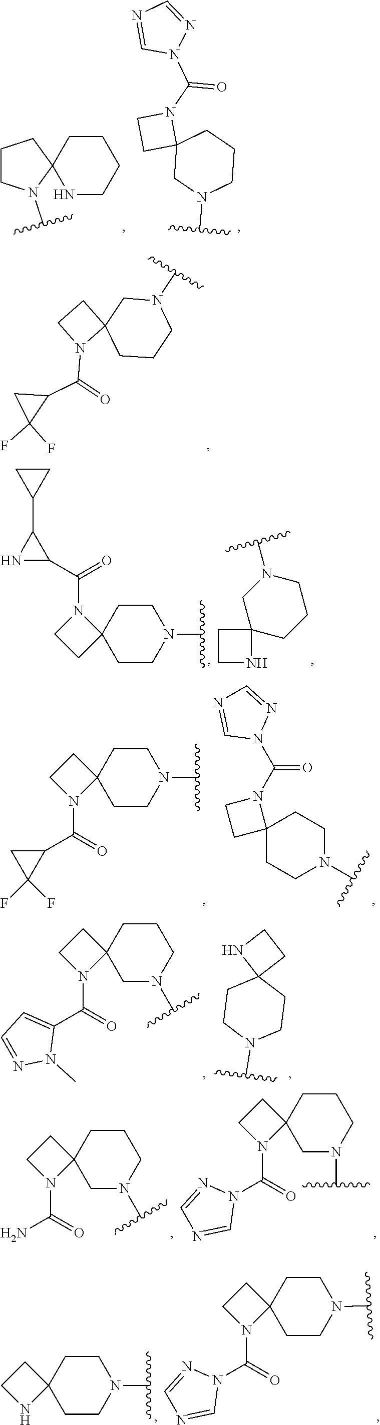

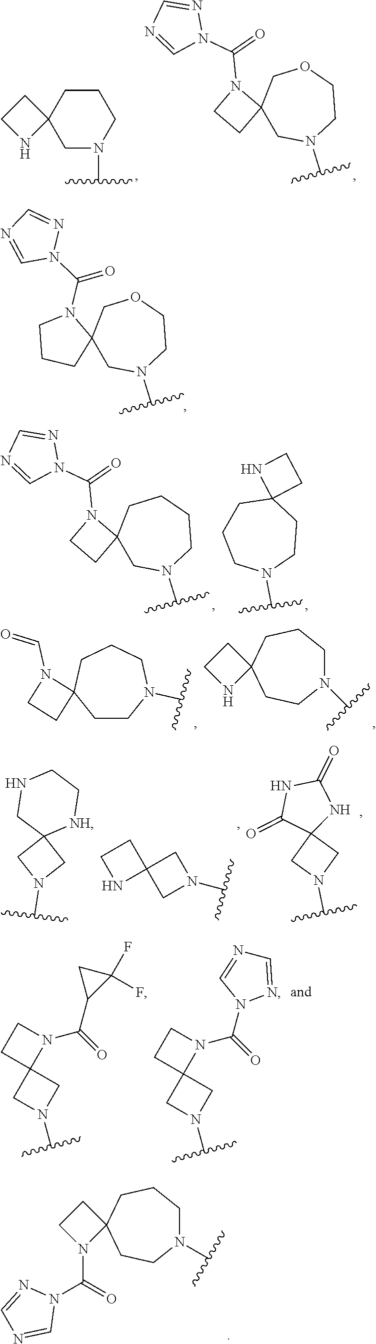

In embodiments of Formula (I), (I-3), (I-4), (II-3), (I′), (Ia), (Ib), (Ic), (Id), (Ii), (Ij), (Ik), (Im), (In), (Io), (Ip), (Iq), (Ir), (II), (II′), (II″), (IV-3), (IV-4), (IV), (IVa), or (IV′), R⁷ is selected from

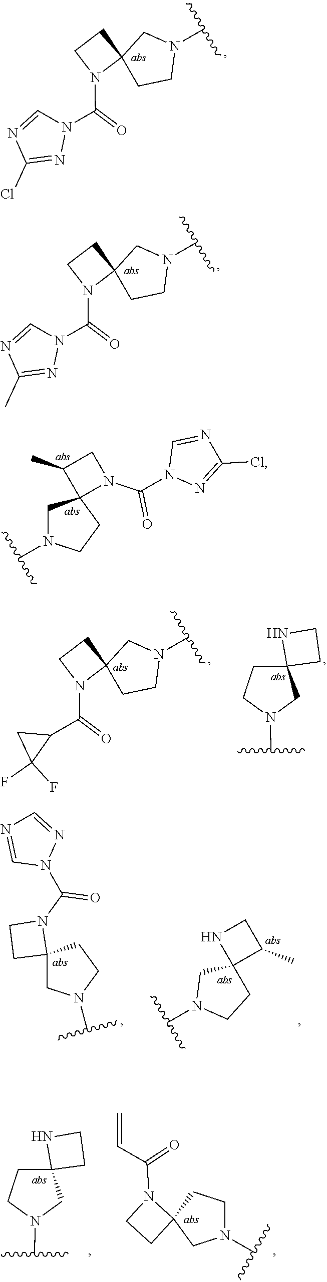

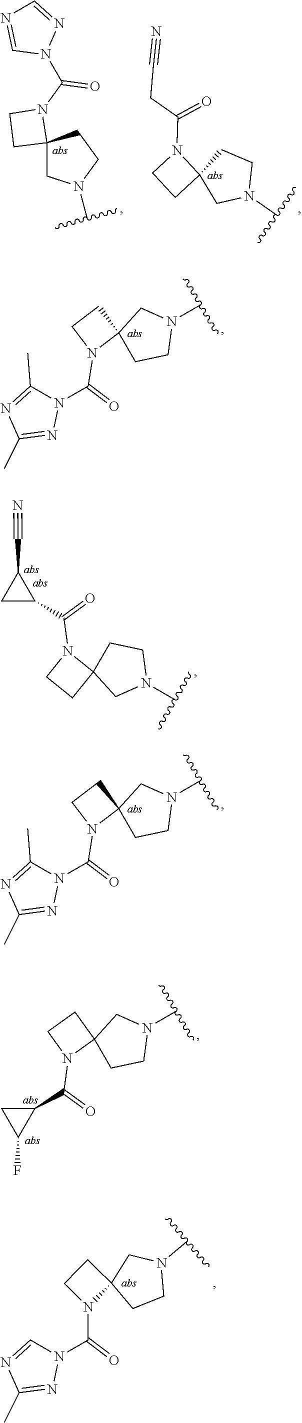

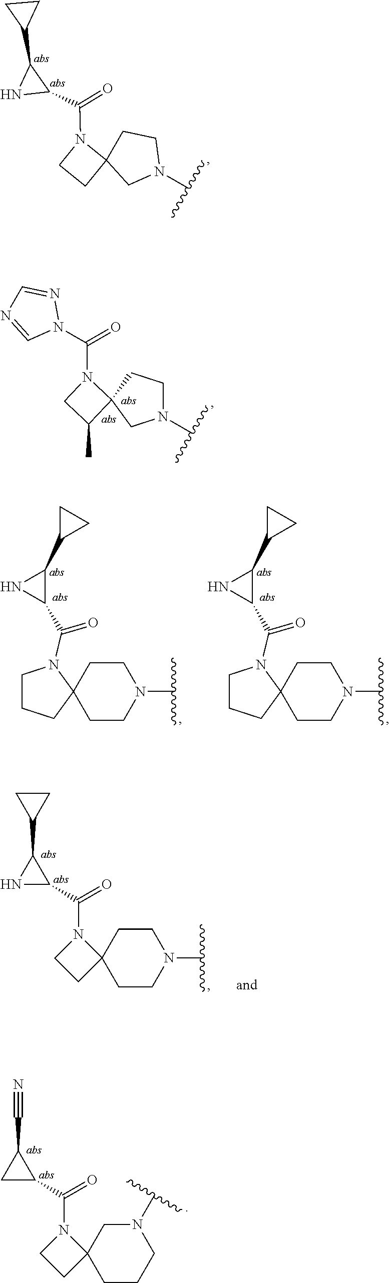 [SMILES: *N1CCC2(CCN2C(=O)[C@@H]2N[C@H]2C2CC2)C1 ; *N1CC[C@]2(C1)[C@@H](C)CN2C(=O)n1cncn1 ; *N1CCC2(CCCN2C(=O)[C@@H]2N[C@H]2C2CC2)CC1 ; *N1CCC2(CCCN2C(=O)[C@@H]2N[C@H]2C2CC2)CC1 ; *N1CCC2(CC1)CCN2C(=O)[C@@H]1N[C@H]1C1CC1 ; *N1CCCC2(CCN2C(=O)[C@@H]2C[C@H]2C#N)C1]

In embodiments of Formula (IV-3), (IV-4), (IV), (IVa), or (IV′), R⁷ is

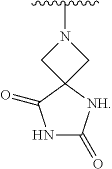 [SMILES: *N1CC2(C1)NC(=O)NC2=O]

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

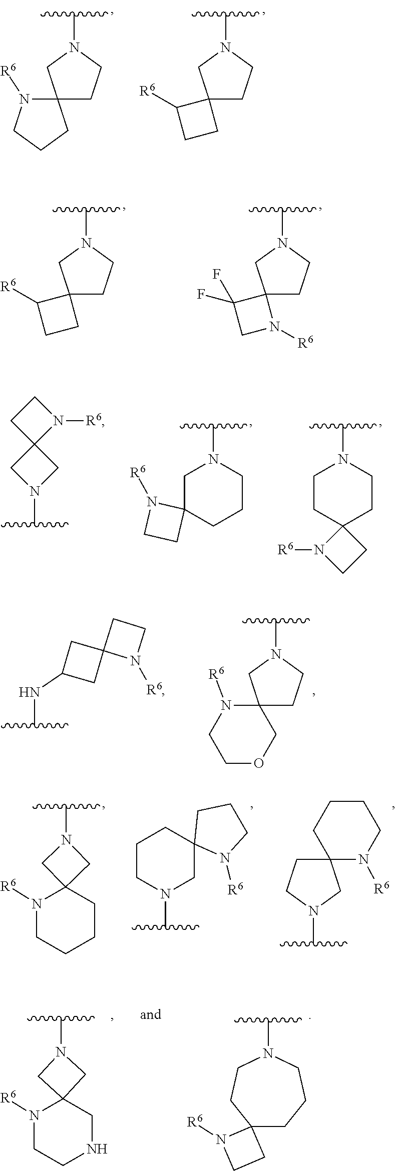

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

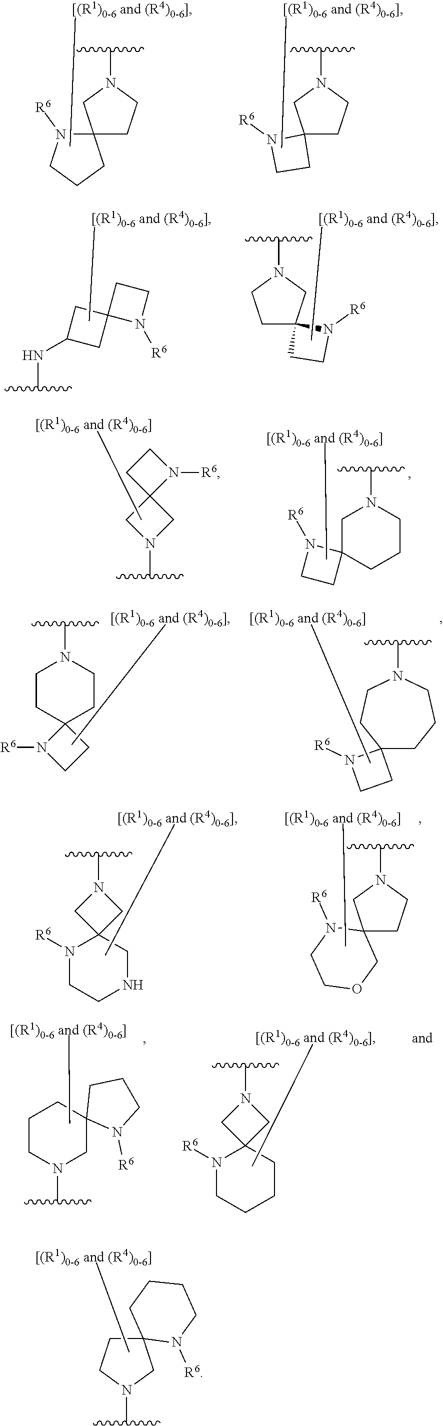

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

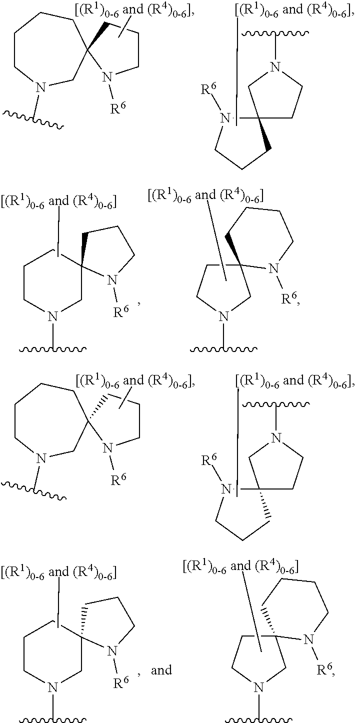

In embodiments of Formula (IV-3), (IV-4), (IV), (IVa), or (IV′), R⁷ is selected from

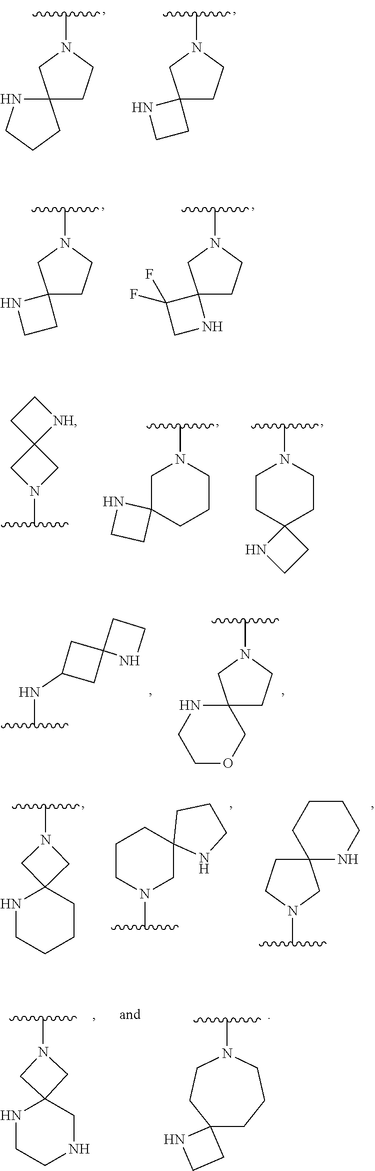

In embodiments of Formula (I), (I-3), (I-4), (II-3), (I′), (Ia), (Ib), (Ic), (Id), (Ie), (If), (Ig), (Ii), (IJ), (Ik), (Im), (In), (Io), (Ip), (Iq), (Ir), (II), (II′), or (II″), R⁷ is selected from

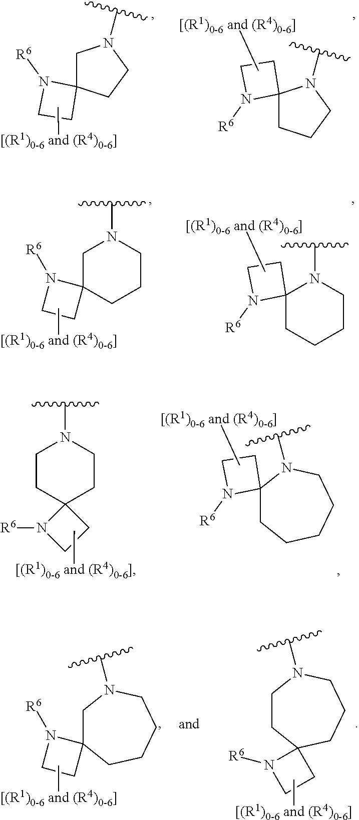

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

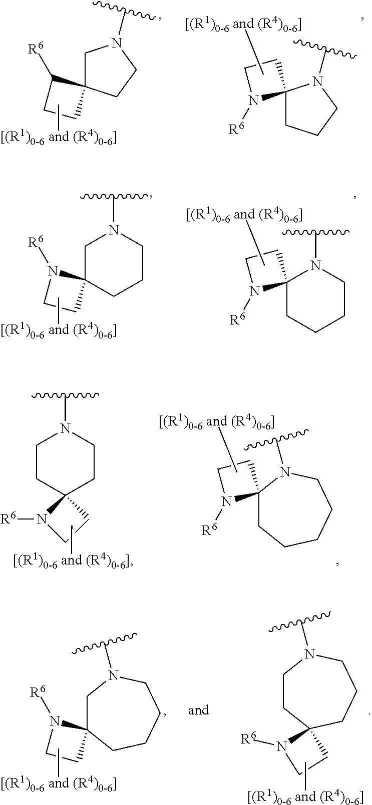

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

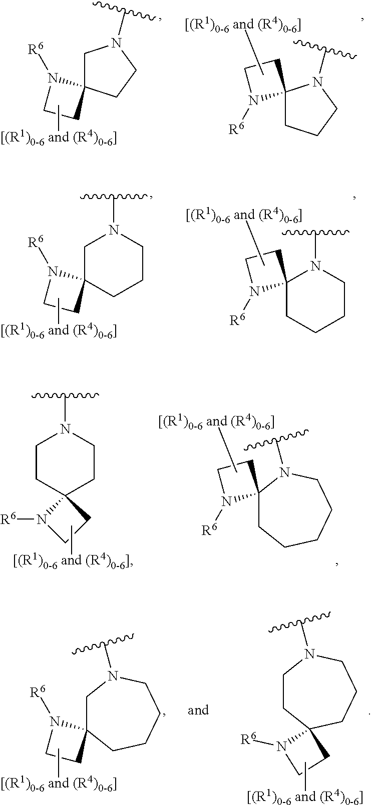

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

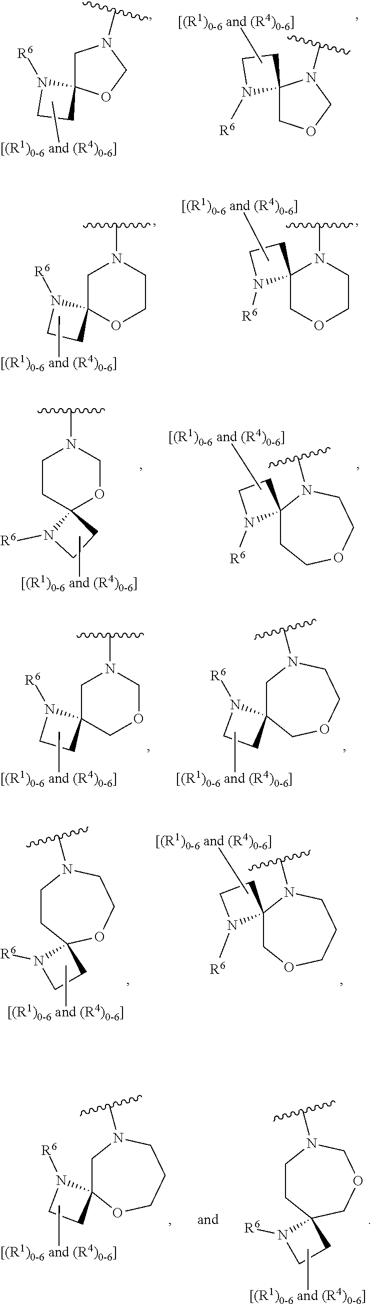

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

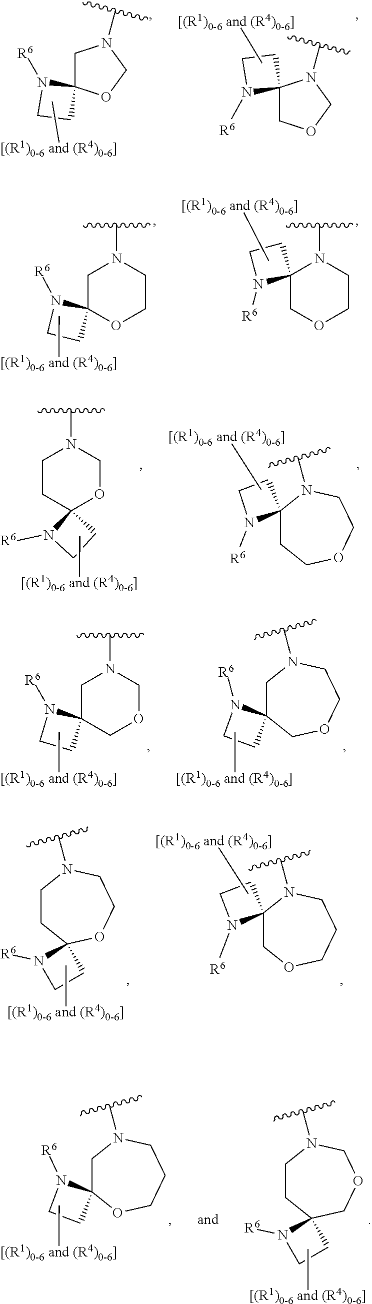

In embodiments of Formula (IV-3), (IV-4), (IV), (IVa), or (IV′), R⁷ is selected from

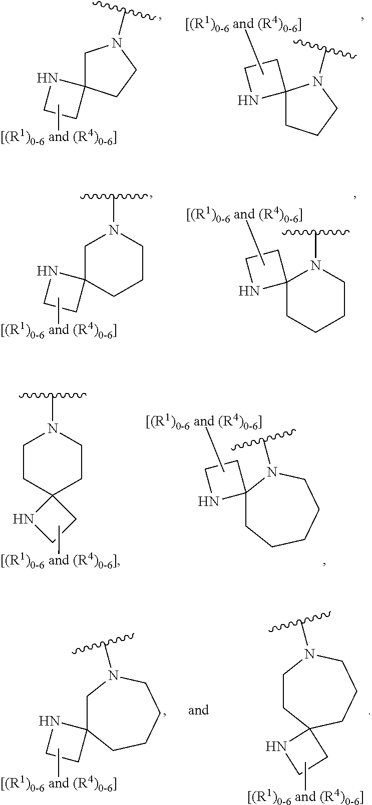

In embodiments of Formula (IV-3), (IV-4), (IV), (IVa), or (IV′), R⁷ is selected from

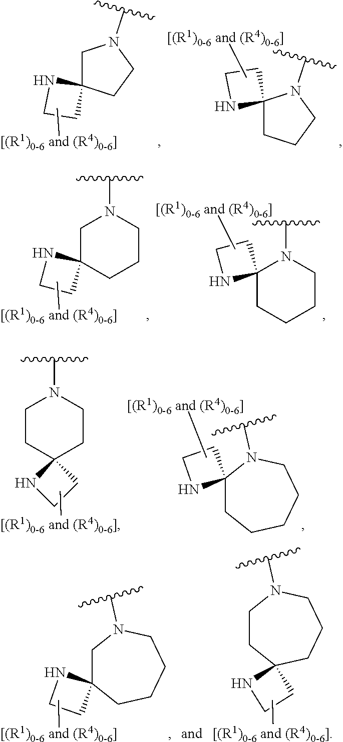

In embodiments of Formula (IV-3), (IV-4), (IV), (IVa), or (IV′), R⁷ is selected from

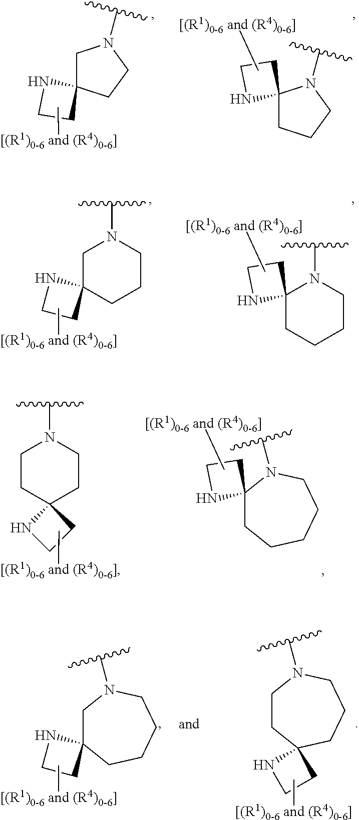

In embodiments of Formula (IV-3), (IV-4), (IV), (IVa), or (IV′), R⁷ is selected from

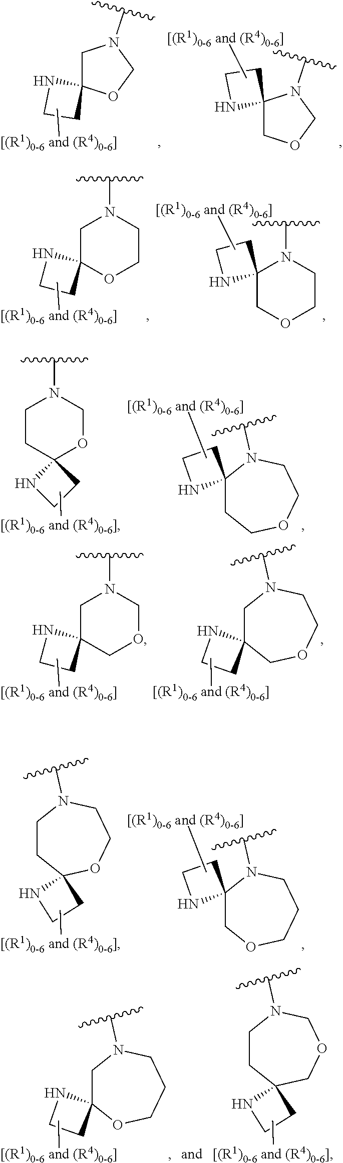

In embodiments of Formula (IV-3), (IV-4), (IV), (IVa), or (IV′), R⁷ is selected from

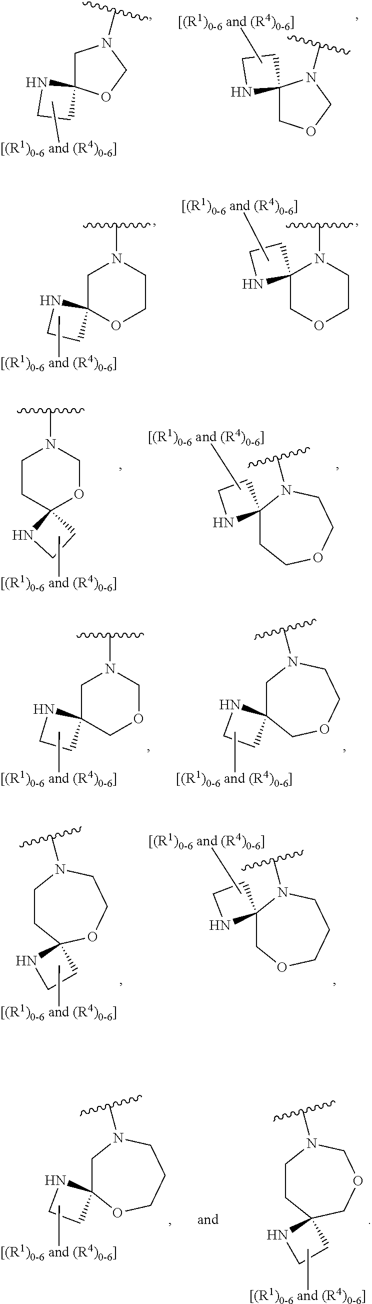

It will be understood that when one or more floating substituent(s) is/are shown extending from one ring in a polycyclic ring system (e.g., fused ring system, bridged ring system, or spirocyclic ring system), the one or more floating substituent(s), may be bonded to the ring from which the one or more floating substituents are shown extending or may be bonded to any other ring in the polycyclic ring system (e.g., fused ring system, spirocyclic ring system, or bridged ring system) and when multiple substituents are represented by the floating substituents, each substituent may be bonded to the same or different rings in the polycyclic ring system, unless indicated otherwise.

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

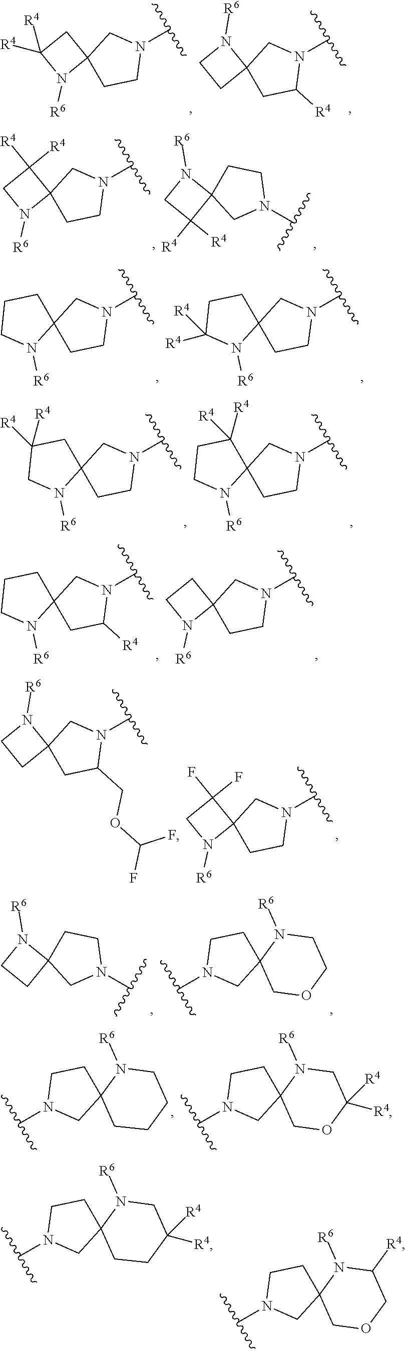

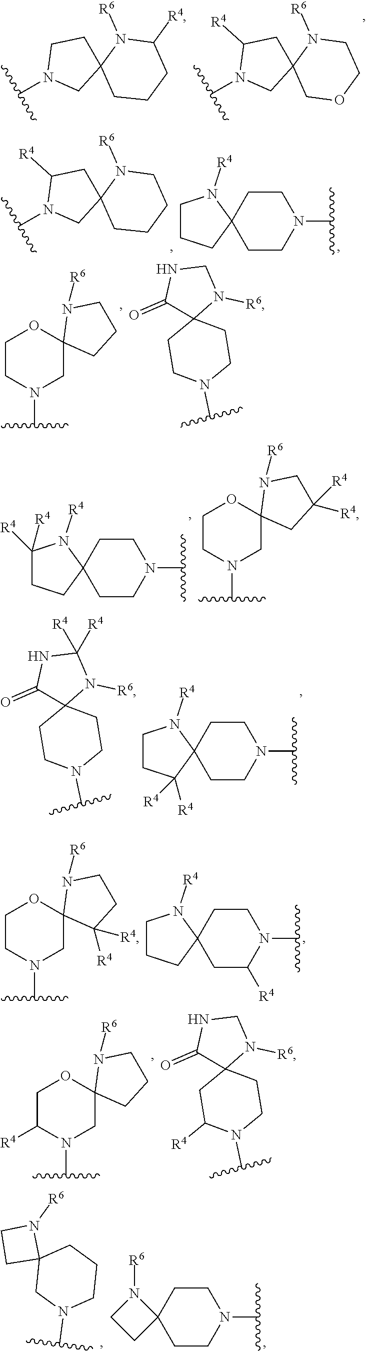

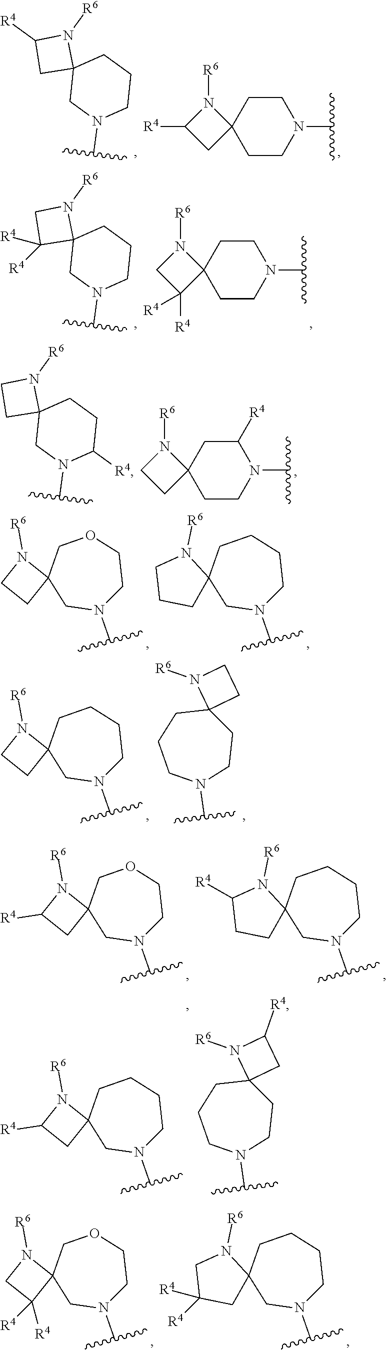

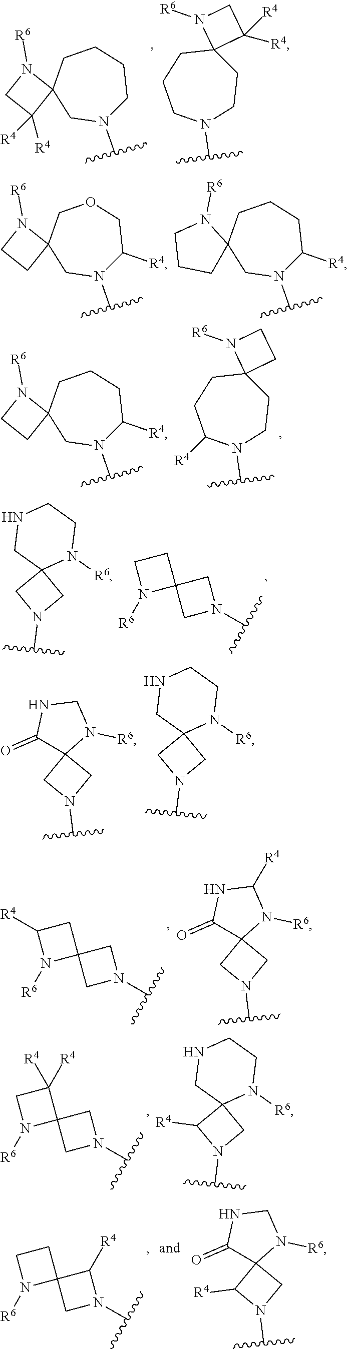

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

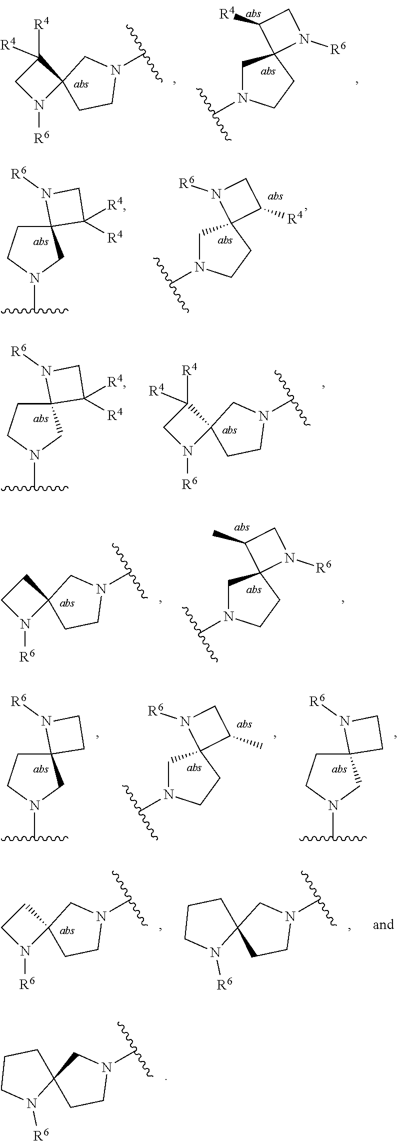

In embodiments of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

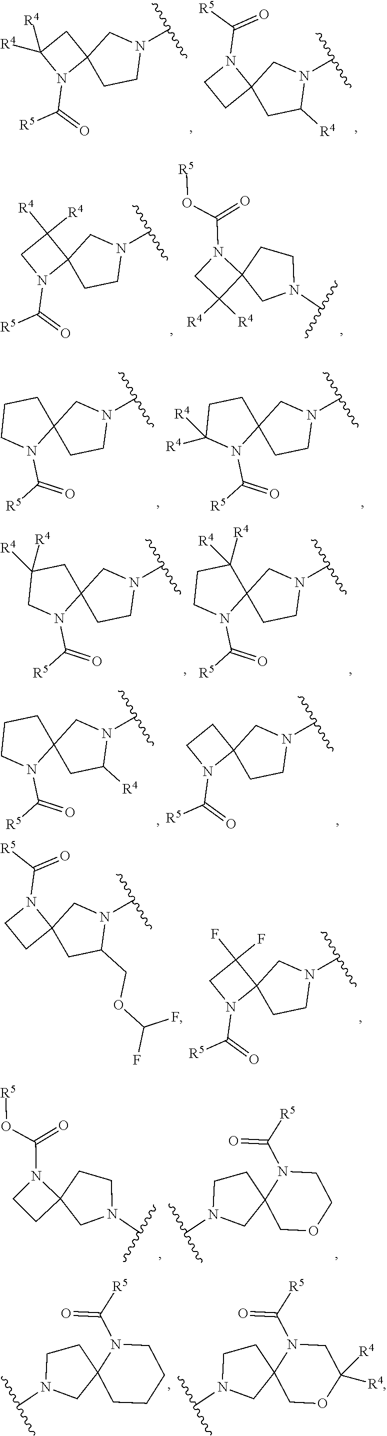

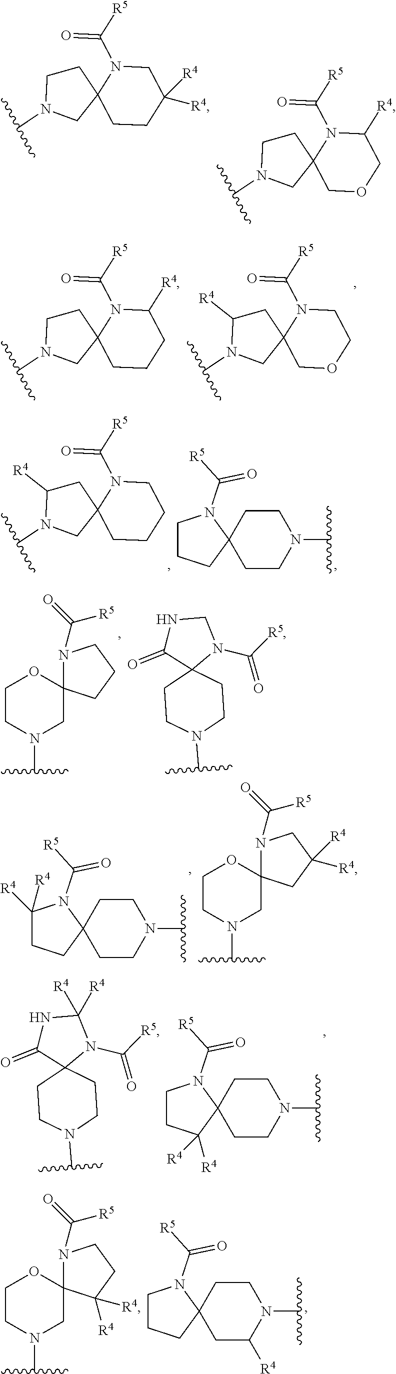

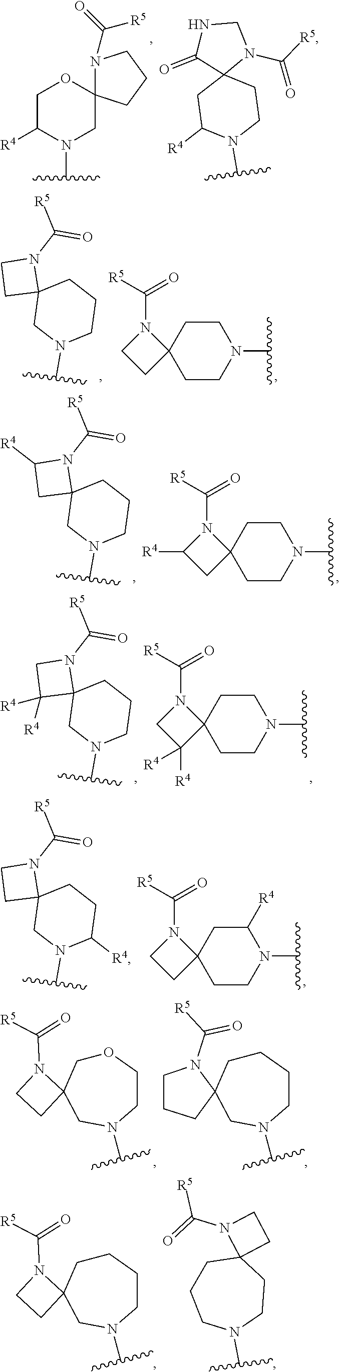

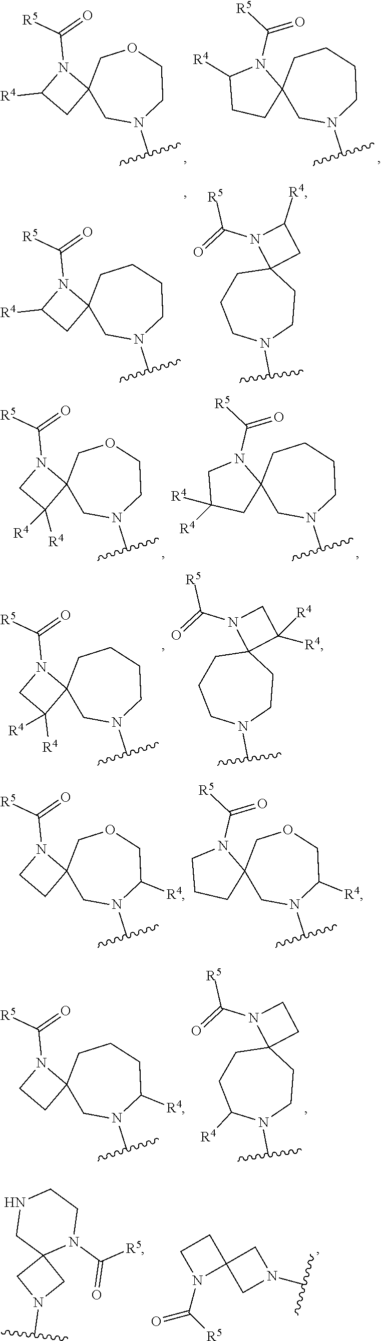

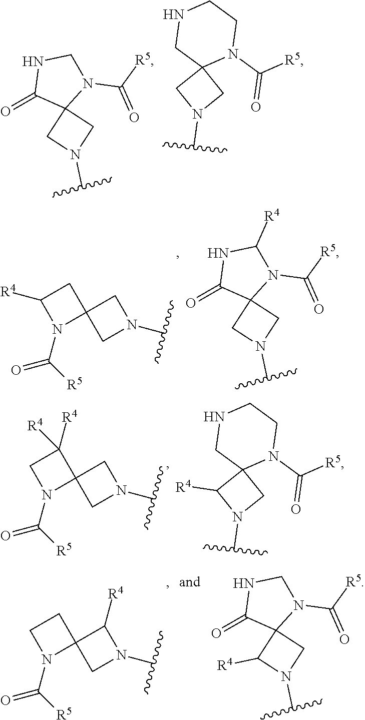

In embodiments of Formula (I), (I-3)₉ (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), or (II″), R⁷ is selected from

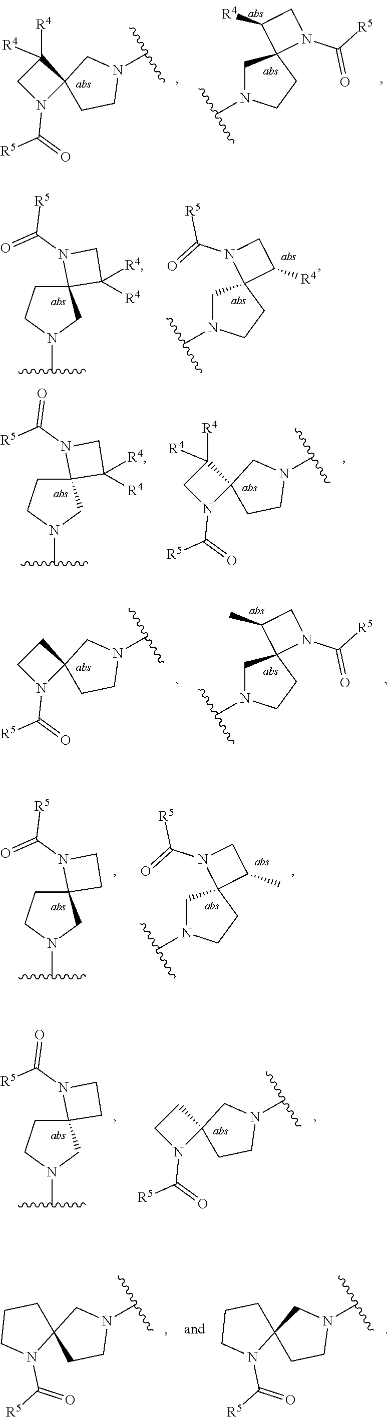

In embodiments of Formula (IV-3), (IV-4), (IV), (IVa), or (IV′), R⁷ is selected from

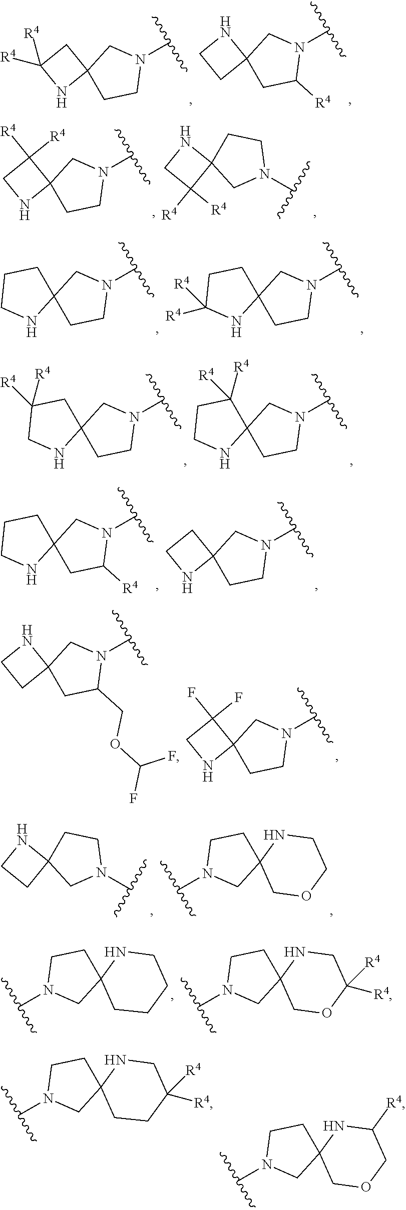

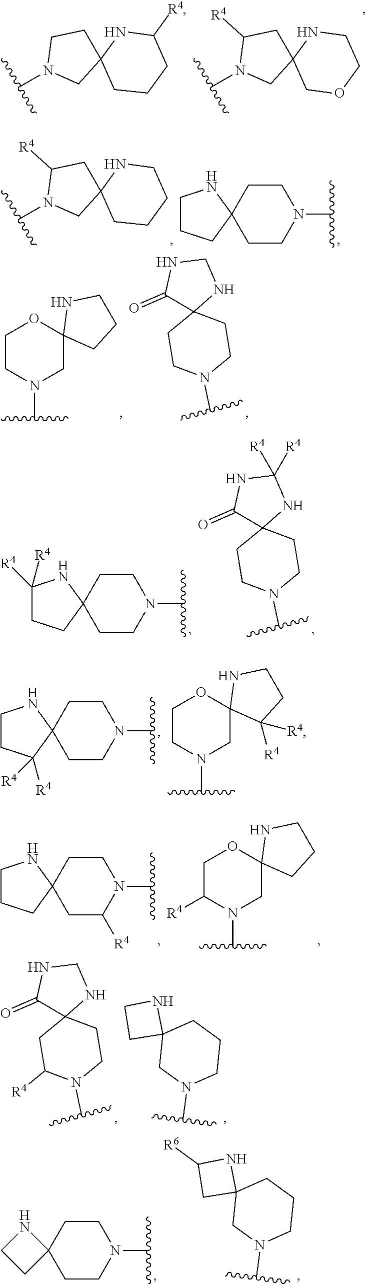

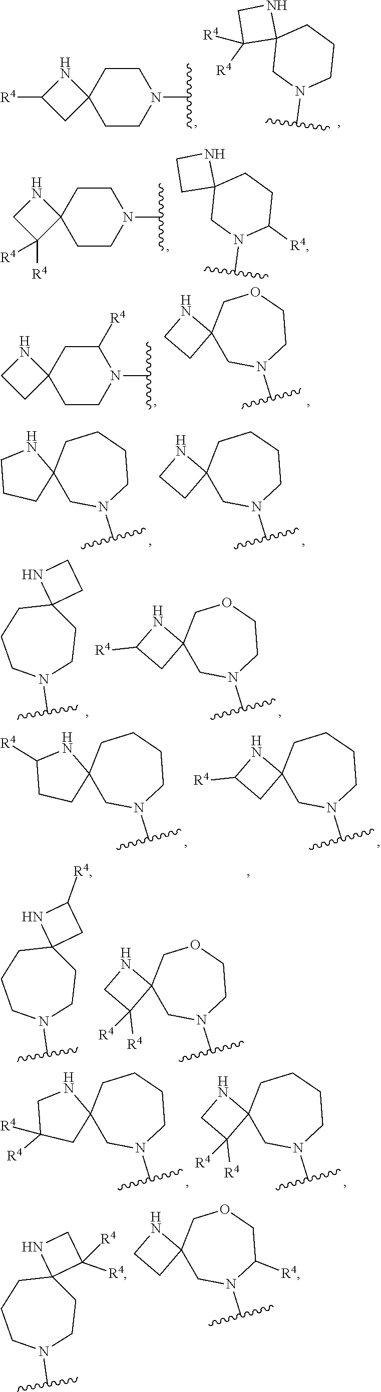

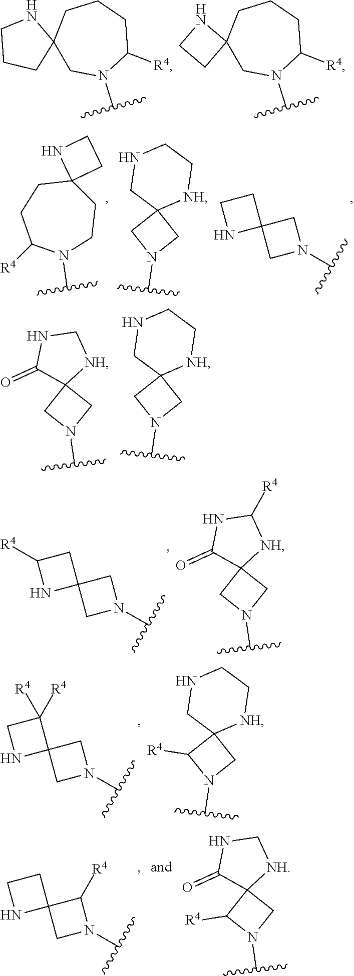

In embodiments of Formula (IV-3), (IV-4), (IV), (IVa), or (IV′), R⁷ is selected from

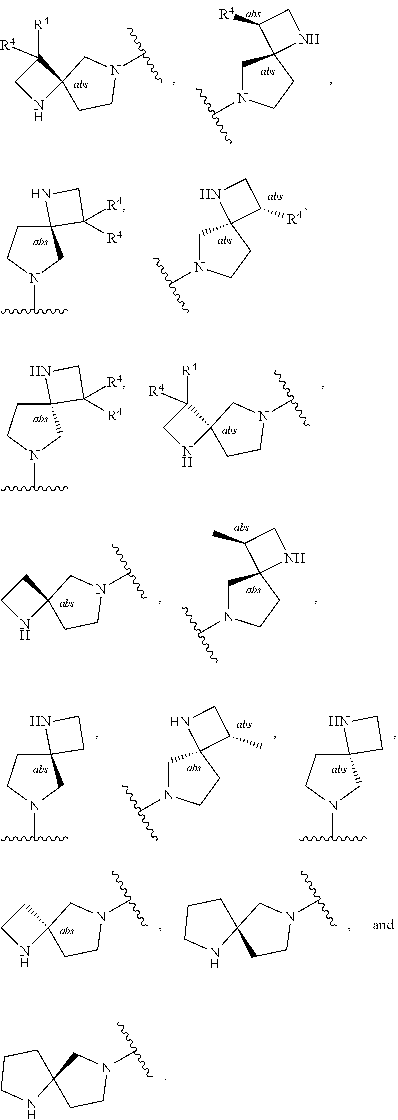

In embodiments of Formula (I), (I-3), (I-4), (II-3), (I′), (IVa), (La), (Ib), (Ic), (Id), (Ie), (If), (Ig), (II), (II′), (II″), (IV-3), (IV-4), (IV), or (IV″), the formula

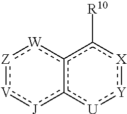

is selected from

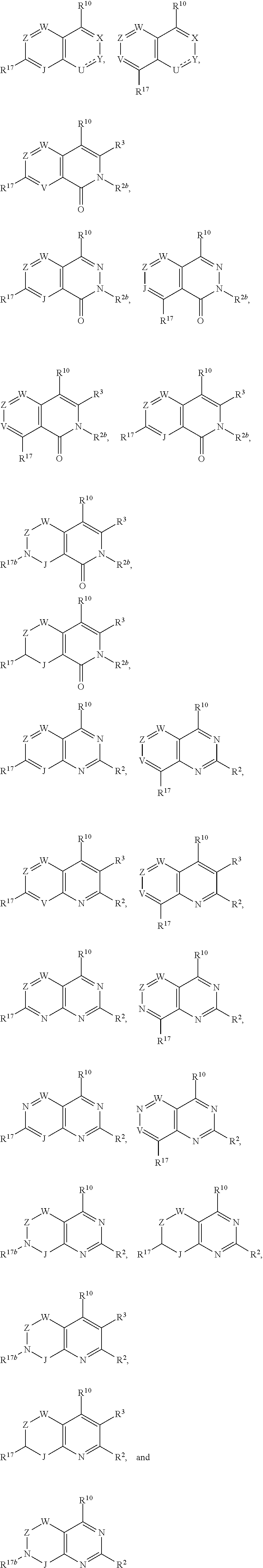

wherein X, Y, U, W, Z, V, J, R²⁹, R^{2b}, R³, R¹⁰, R¹⁷, and R^{17b} are as described herein. In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

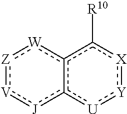

is selected from

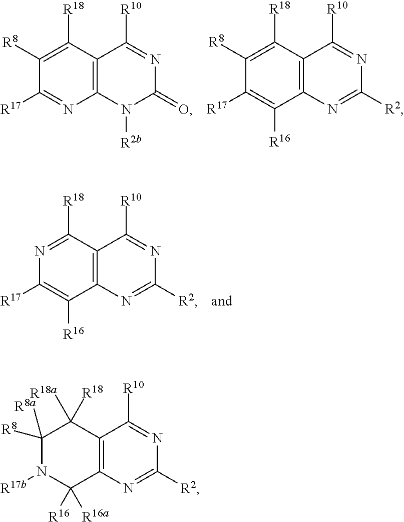

wherein X, Y, U, W, Z, V, J, R², R^{2b}, R¹⁰, R¹, R^{8a}, R¹⁶, R^{1a}, R¹⁸, R^{18a}, R¹⁷, and R¹⁷ are as described herein.

In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

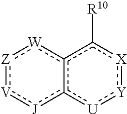

is

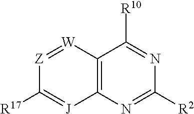

wherein W, Z, J, R², R¹⁰, and R¹⁷ are as described herein.

In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

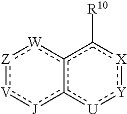

is

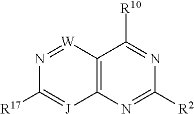

wherein W, J, R², R¹⁰, and R¹⁷ are as described herein.

In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), (IV′), or (IV″), the formula

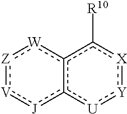

is

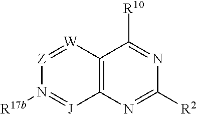

wherein W, Z, J, R², R¹⁰, and R¹⁷ are as described herein.

In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

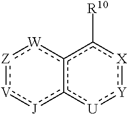

is

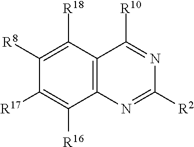

wherein R², R¹⁰, R⁸, R¹⁶, R¹⁸, and R¹⁷ are as described herein.

In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

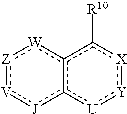

is

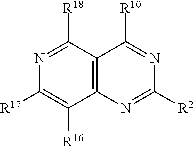

wherein R², R¹⁰, R¹⁶, R¹⁸, and R¹⁷ are as described herein.

In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

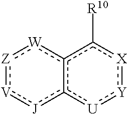

is

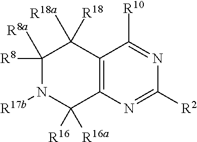

wherein R², R¹⁰, R⁸, R^{8a}, R¹⁶, R^{16a}, R¹⁸, R^{18a}, and R¹¹ are as described herein.

In embodiments of Formula (II), (II′), (II″), (IV-3), (IV-4), (IV), (IVa), or (IV″), the formula

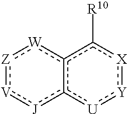

is selected from

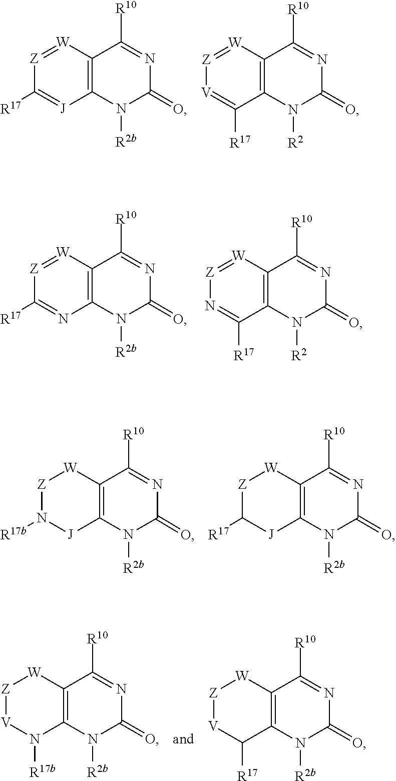

wherein X, Y, U, W, Z, V, J, R², R^{2b}, R³, R¹⁰, R¹⁷, and R¹⁷ are as described herein.

In embodiments of Formula (II), (II′), (II″), (IV-3), (IV-4), (IV), (IVa), or (IV″), the formula

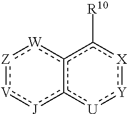

is

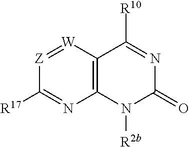

wherein W, Z, R^{2b}, R¹⁰, and R¹⁷ are as described herein.

In embodiments of Formula (II), (II′), (II″), (IV-3), (IV-4), (IV), (IVa), or (IV″), the formula

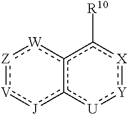

is

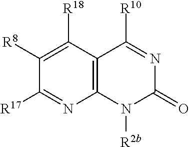

wherein R^{2b}, R¹⁰, R⁸, R¹⁸, and R¹⁷ are as described herein. In embodiments of Formula (I), (I-3), (I-4), (II-3), (I′), (IVa), (Ia), (Ib), (Ic), (Id), (Ie), (If), (Ig), (II), (II′), (II″), (IV-3), (IV-4), (IV), or (IV″), the formula

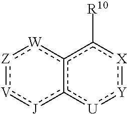

is

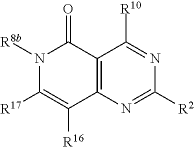

wherein R², R¹⁰, R^{8b}, R¹⁶, and R¹ⁱ are as described herein.

In an aspect is provided a compound selected from

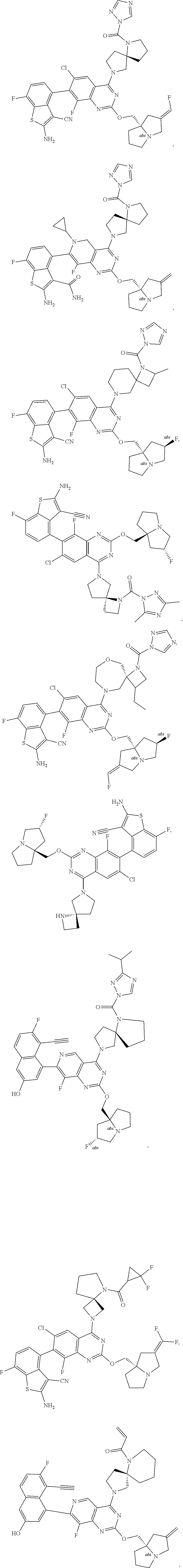

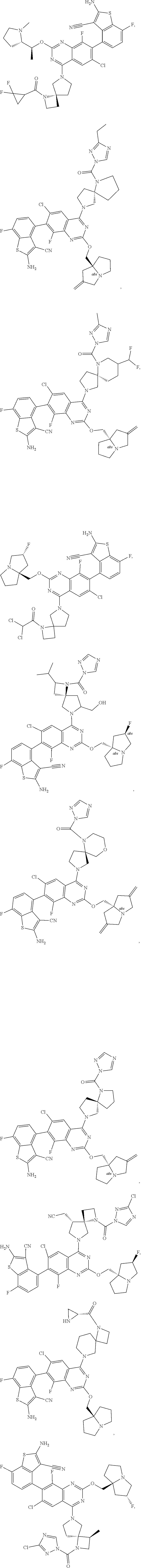

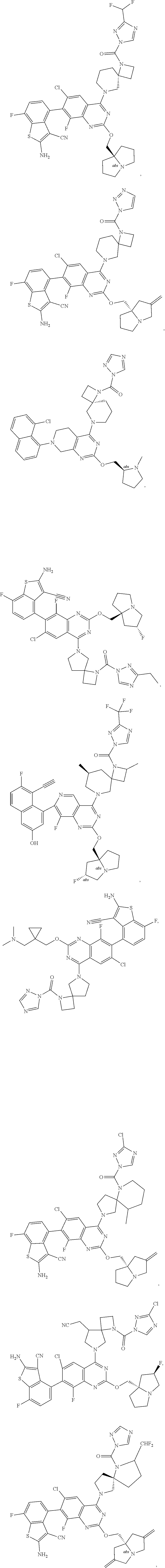

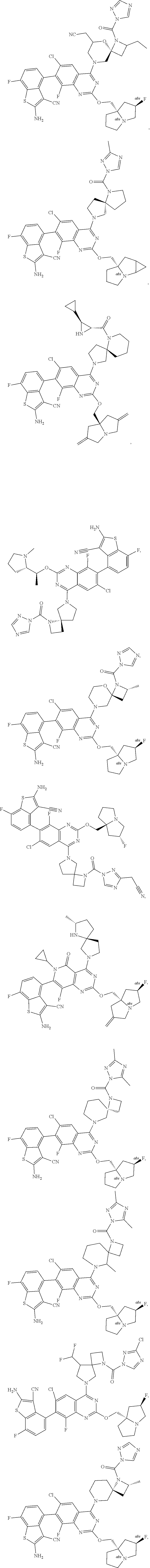

[chemical structure]

[chemical structure]

[chemical structure]

[chemical structure]

[chemical structure]

[chemical structure]

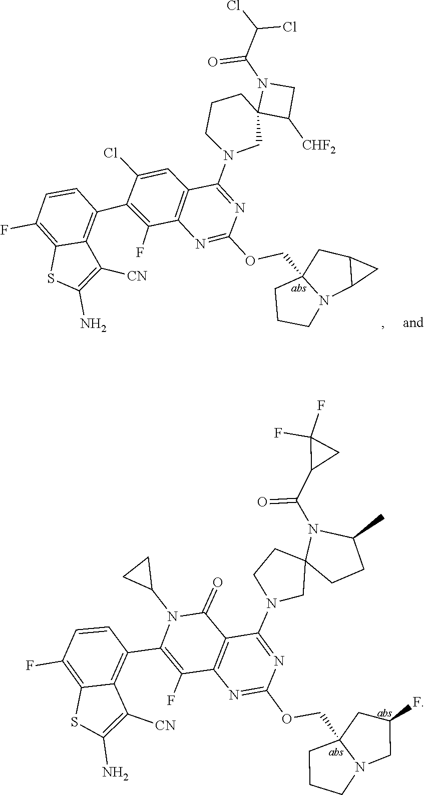 [SMILES: N#Cc1c(N)sc2c(F)ccc(-c3c(Cl)cc4c(N5CCC[C@]6(C5)C(C(F)F)CN6C(=O)C(Cl)Cl)nc(OC[C@@]56CCCN5C5CC5C6)nc4c3F)c12 ; C[C@H]1CCC2(CCN(c3nc(OC[C@@]45CCCN4C[C@H](F)C5)nc4c(F)c(-c5ccc(F)c6sc(N)c(C#N)c56)n(C5CC5)c(=O)c34)C2)N1C(=O)C1CC1(F)F]

In embodiments of the compounds recited in the paragraph immediately above, wherein atropisomers may be present, each compound is a single atropisomer (e.g., equivalent to R atropisomer) at the atom corresponding to the V atom bonded to R¹⁷ of Formula (I). In embodiments of the compounds recited in the paragraph immediately above, wherein atropisomers may be present, each compound is a single atropisomer (e.g., equivalent to S atropisomer) at the atom corresponding to the V atom bonded to R¹⁷ of Formula (I). In embodiments of the compounds recited in the paragraph immediately above, wherein atropisomers may be present, each compound is a single atropisomer (e.g., having the equivalent orientation as

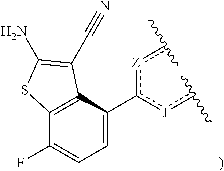

at the atom corresponding to the V atom bonded to R¹⁷ of Formula (I). In embodiments of the compounds recited in the paragraph immediately above, wherein atropisomers may be present, each compound is a single atropisomer (e.g., having the equivalent orientation as

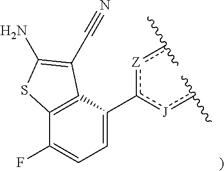

at the atom corresponding to the V atom bonded to R¹⁷ of Formula (I). In embodiments of the compounds recited in the paragraph immediately above, wherein atropisomers may be present, each compound is a single atropisomer (e.g., having the equivalent orientation as

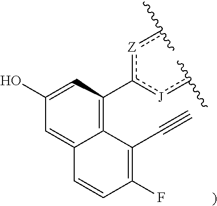

at the atom corresponding to the V atom bonded to R¹⁷ of Formula (I). In embodiments of the compounds recited in the paragraph immediately above, wherein atropisomers may be present, each compound is a single atropisomer (e.g., having the equivalent orientation as

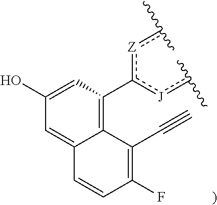

at the atom corresponding to the V atom bonded to R¹⁷ of Formula (I).

In an aspect is provided a compound selected from,

[chemical structure]

[chemical structure]

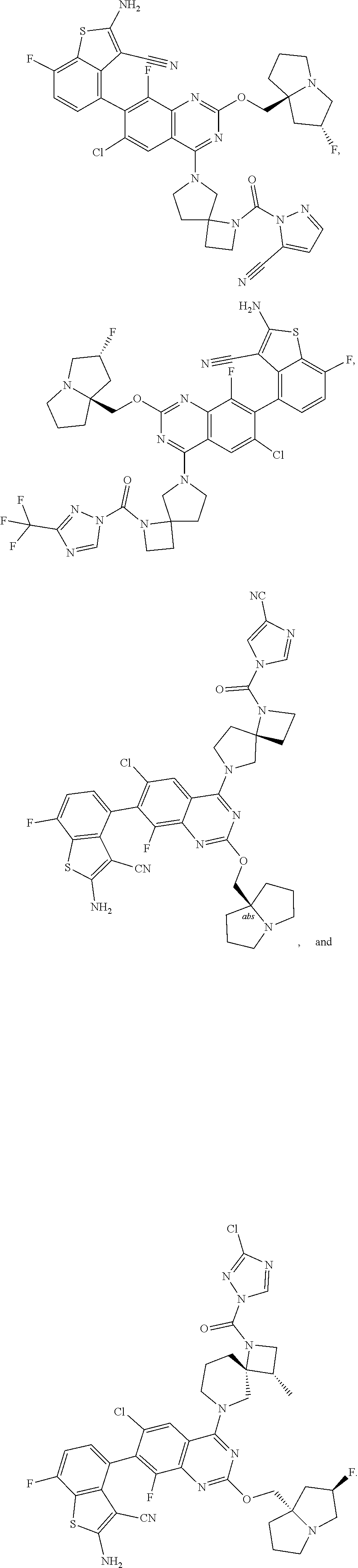

In embodiments of the compounds recited in the paragraph immediately above, each compound is a substantially pure single regioisomer at the positions corresponding to the attachment points of R⁵ to L² and R⁵ to R^{20k} in Formula (I) The single regioisomer shown in the compounds above is merely for simplicity of representation and it will be understood that in one embodiment, each compound is the regioisomer shown and in additional embodiments, the regioisomer is each of the alternative regioisomers. In embodiments of the compounds recited in the paragraph immediately above, each compound is a mixture of regioisomers at the positions corresponding to the attachment points of R⁵ to L² and R⁵ to R^{20k} in Formula (I). The single regioisomer shown in the compounds above is merely for simplicity of representation and it will be understood that embodiments, the compound is mixture of all possible regioisomers. In embodiments of the compounds recited in the paragraph immediately above, each compound is a partially purified mixture of regioisomers at the positions corresponding to the attachment points of R⁵ to L² and R⁵ to R^{20k} in Formula (I) (i.e., some but not all regioisomers are provided). In embodiments of the compounds recited in the paragraph immediately above, each compound is a single atropisomer (e.g., equivalent to R atropisomer) at the atom corresponding to the V atom bonded to R¹⁷ of Formula (I) and may optionally be a single, substantially single, partially purified, or mixture of regioisomers at the positions corresponding to the attachment points of R⁵ to L² and R⁵ to R^{20k} in Formula (I). In embodiments of the compounds recited in the paragraph immediately above, each compound is a single atropisomer (e.g., equivalent to S atropisomer) at the atom corresponding to the V atom bonded to R¹⁷ of Formula (I) and may optionally be a single, substantially single, partially purified, or mixture of regioisomers at the positions corresponding to the attachment points of R⁵ to L² and R⁵ to R^{20k} in Formula (I). In embodiments of the compounds recited in the paragraph immediately above, wherein atropisomers may be present, each compound is a single atropisomer (e.g., having the equivalent orientation as

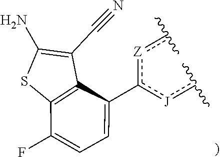

at the atom corresponding to the V atom bonded to R¹⁷ of Formula (I) and may optionally be a single, substantially single, partially purified, or mixture of regioisomers at the positions corresponding to the attachment points of R⁵ to L² and R⁵ to R^{20k} in Formula (I). In embodiments of the compounds recited in the paragraph immediately above, wherein atropisomers may be present, each compound is a single atropisomer (e.g., having the equivalent orientation as

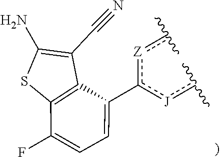

at the atom corresponding to the V atom bonded to R¹⁷ of Formula (I) and may optionally be a single, substantially single, partially purified, or mixture of regioisomers at the positions corresponding to the attachment points of R⁵ to L² and R⁵ to R^{2k} in Formula (I). In embodiments of the compounds recited in the paragraph immediately above, wherein atropisomers may be present, each compound is a single atropisomer (e.g., having the equivalent orientation as

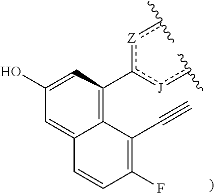

at the atom corresponding to the V atom bonded to R¹⁷ of Formula (I) and may optionally be a single, substantially single, partially purified, or mixture of regioisomers at the positions corresponding to the attachment points of R⁵ to L² and R⁵ to R^{20k} in Formula (I). In embodiments of the compounds recited in the paragraph immediately above, wherein atropisomers may be present, each compound is a single atropisomer (e.g., having the equivalent orientation as

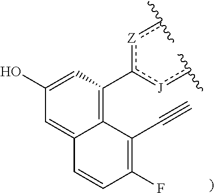

at the atom corresponding to the V atom bonded to R¹⁷ of Formula (I) and may optionally be a single, substantially single, partially purified, or mixture of regioisomers at the positions corresponding to the attachment points of R⁵ to L² and R⁵ to R^{20k} in Formula (I). In embodiments, the compound is a compound described herein, including in an aspect, embodiment, example, table, scheme, method, or composition.

In an aspect is provided a compound having the formula A-L^{AB}-B wherein
A is a monovalent form of a compound described herein;
L^{A} is a covalent linker bonded to A and B; and
B is a monovalent form of a degradation enhancer.
In embodiments of a compound described herein, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885.

In embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1.

In embodiments, L^{AB} is -L^{AB1}-L^{AB2}-L^{AB3}-L^{AB4}-L^{AB5}-;
L^{AB1}, L^{AB2}, L^{AB3}, L^{AB4}, and L^{AB5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆alkylene, (—O—C₁₋₆alkyl)₂-, (—C₁₋₆alkyl-O)₂—, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁heteroarylene, wherein C₁₋₆ alkylene, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁ heteroarylene, are optionally substituted with one, two, or three R^{20j}; wherein each C₁₋₆alkyl of (—O—C₁₋₆alkyl)_{z}- and (—C₁₋₆ alkyl-O)_{z}— is optionally substituted with one, two, or three R^{20j};
z is independently an integer from 0 to 10;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20f};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
each R^{20d}, R²⁰%, R^{20f}, and R^{20j} are each independently selected from halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl.

In embodiments, L^{A} is —(O—C₂alkyl)_{z}- and z is an integer from 1 to 10.

In embodiments, L^{A} is —(C₂alkyl-O—)_{z}— and z is an integer from 1 to 10.

In embodiments, L^{A} is —(CH₂)_{zz1}L¹²(CH₂O)_{zz2}—, wherein L¹² is a bond, a 5 or 6 membered heterocycloalkylene or heteroarylene, phenylene, —(C₂-C₄)alkynylene, —SO₂— or —NH—; and zz1 and zz2 are independently an integer from 0 to 10.

In embodiments, L^{A} is —(CH₂)_{zz1}(CH₂O)_{zz2}—, wherein zz1 and zz2 are each independently an integer from 0 to 10.

In embodiments, L^{AB} is a PEG linker.

In embodiments, B is a monovalent form of a compound selected from

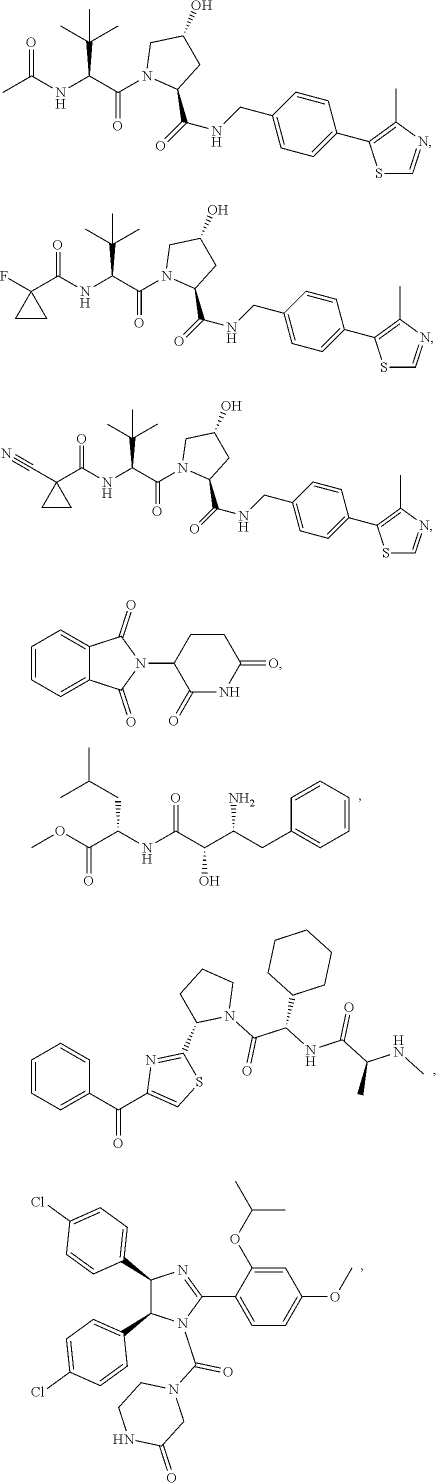

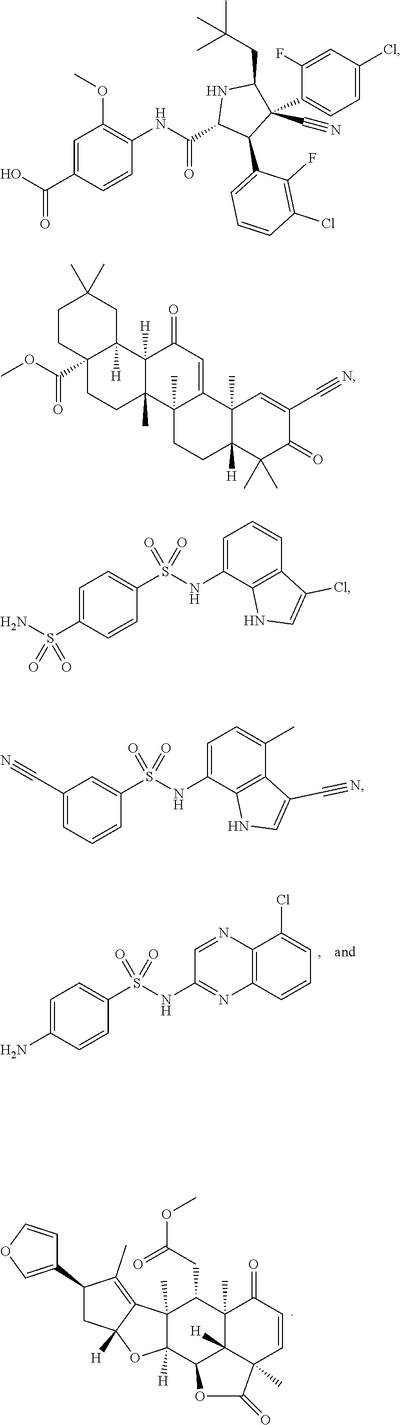

In embodiments, B is a monovalent form of a compound selected from

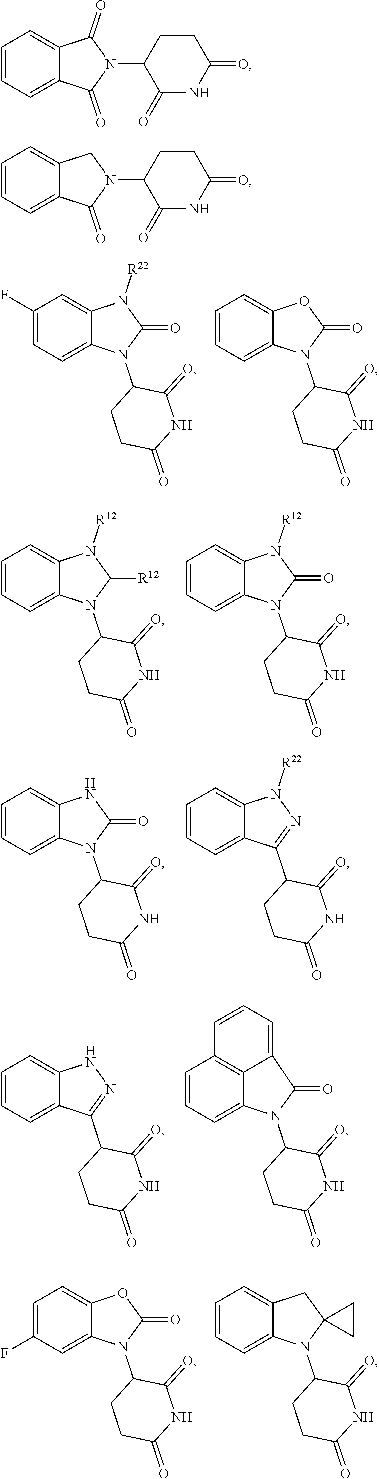

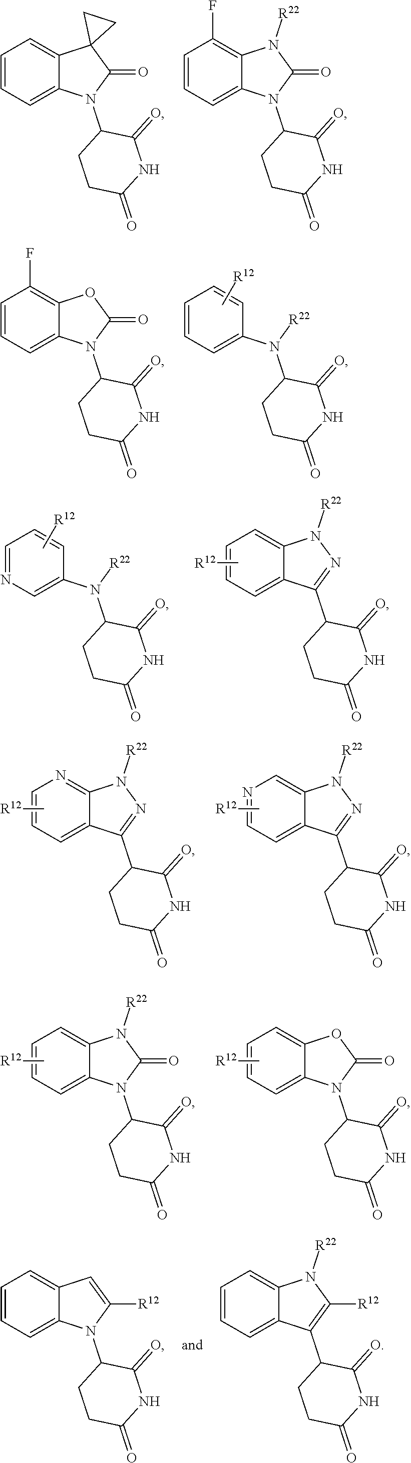

In some embodiments, the compound of formula A-L-B is selected from NI-12

[chemical structure]

or a pharmaceutically acceptable salt or solvate thereof.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

In some embodiments, a compound of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), (IVa), or (IV′), is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, the present disclosure provides an atropisomer of a compound of Formula (I), (I-3), (I-4), (II-3), (I′), an embodiment thereof. In some embodiments, the atropisomer is provided in enantiomeric excess. In some embodiments, the atropisomer is provided in at least 80% enantiomeric excess, such as at least 85%, at least 90%, at least 95%, at least 96%, at least 97%, at least 98%, at least 99%, or at least 99.9% enantiomeric excess. In some embodiments, the compound of Formula (I), (I-3), (I-4), (II-3), (I′), (Ia), (Ib), (Ic), (Id), (Ie), (If), (Ig), (Ii), (Ij), (Ik), (Im), (In), (Io), (Ip), (Iq), (Ir), (II), (II′), (II″), (IV-3), (IV-4), (IV), (IVa), or (IV′), or an embodiment thereof, is preferably used as a non-racemic mixture, wherein one atropisomer is present in excess of its corresponding enantiomer or epimer. Typically, such mixture will contain a mixture of the two isomers in a ratio of at least 9:1, preferably at least 19:1. In some embodiments, the atropisomer is provided in at least 96% enantiomeric excess, meaning the compound has less than 2% of the corresponding enantiomer. In some embodiments, the atropisomer is provided in at least 96% diastereomeric excess, meaning the compound has less than 2% of the corresponding diastereomer.

The term “atropisomers” refers to conformational stereoisomers which occur when rotation about a single bond in the molecule is prevented, restricted, or greatly slowed as a result of steric interactions with other parts of the molecule and wherein the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom. Atropisomers are typically considered stable if the barrier to interconversion is high enough to permit the atropisomers to undergo little or no interconversion at room temperature for a least a week, preferably at least a year. In some embodiments, an atropisomeric compound of the disclosure does not undergo more than about 5% interconversion to its opposite atropisomer at room temperature during one week when the atropisomeric compound is in substantially pure form, which is generally a solid state. In some embodiments, an atropisomeric compound of the disclosure does not undergo more than about 5% interconversion to its opposite atropisomer at room temperature (approximately 25° C.) during one year. The present chemical entities, pharmaceutical compositions, and methods are meant to include all such possible atropisomers, including racemic mixtures, diastereomeric mixtures, epimeric mixtures, optically pure forms of single atropisomers, and intermediate mixtures.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁷O, ¹⁸O, ³¹P, ³²P, ³⁵S, ¹⁸F, and ³⁶Cl, respectively. Compounds described herein, and pharmaceutically acceptable salts, esters, solvate, hydrates, or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{th} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. In some embodiments, the following synthetic method may be utilized.

### Methods

The compounds, a pharmaceutically acceptable salt or solvate thereof disclosed herein, have a wide range of applications in therapeutics, diagnostics, and other biomedical research.

In an aspect is provided a method of treating cancer in a subject in need thereof, comprising administering to the subject a therapeutically effective amount of a compound described herein, or a pharmaceutically acceptable salt or solvate thereof. In an aspect is provided a method of treating cancer in a subject comprising a Ras mutant (e.g., G12C, G12D, G12S, G1V, G13C, or G13D) protein, comprising inhibiting amplified wildtype Ras or the Ras mutant (e.g., G12C, G12D, G12S, G1V, G13C, or G13D) protein of said subject by administering to said subject a compound, wherein compound is characterized in that upon contacting the Ras mutant (e.g., G12C, G12D, G12S, G1V, G13C, or G13D) protein, said Ras mutant (e.g., G12C, G12D, G12S, G1V, G13C, or G13D) protein activity or function is inhibited (e.g., partially inhibited or completely inhibited), such that said inhibited Ras mutant (e.g., G12C, G12D, G12S, G1V, G13C, or G13D) protein exhibits reduced Ras signaling output (e.g., compared to corresponding Ras protein not contacted by the compound).

In some embodiments, provided is a method of reducing Ras signaling output in a cell by contacting the cell with a compound described herein. A reduction in Ras signaling can be evidenced by one or more members of the following: (i) an increase in steady state level of GDP-bound modified protein or a decrease in steady state level of GTP-bound modified protein; (ii) a reduction of phosphorylated AKTs473, (iii) a reduction of phosphorylated ERKT202/y204, (iv) a reduction of phosphorylated S6S235/236, and (v) reduction of cell growth of a tumor cell expressing a Ras mutant (e.g., G12C, G12D, G12S, G1V, G13C, or G13D) protein, and (vi) reduction in Ras interaction with a Ras-pathway signaling protein.

In an aspect is provided a method of treating cancer in a subject comprising a Ras mutant protein, the method comprising: modifying the Ras mutant protein of said subject by administering to said subject a compound, wherein the compound is characterized in that upon contacting the Ras mutant protein, said Ras mutant protein is modified covalently at a residue corresponding to reside 12 of SEQ ID No: 1, such that said modified Ras mutant protein exhibits reduced Ras signaling output.

In an aspect is provided a method of modulating signaling output of a Ras protein, comprising contacting a Ras protein with an effective amount of a compound described herein, or a pharmaceutically acceptable salt or solvate thereof, thereby modulating the signaling output of the Ras protein.

In embodiments, the modified Ras mutant protein comprises a compound described herein covalently bonded to the amino acid residue corresponding to position 12 or 13 of SEQ ID No: 2, wherein the Ras mutant protein is a human protein selected from KRas G12D, KRas G12C, KRas G12S, KRas G13D, KRas G13C, and KRas G13S. In embodiments, the modified Ras mutant protein comprises a compound described herein covalently bonded to the amino acid residue corresponding to position 12 or 13 of SEQ ID No: 6, wherein the Ras mutant protein is a mammalian Ras protein (including human protein) selected from NRas G12D, NRas G12C, NRas G12S, NRas G13D, NRas G13C, and NRas G13S. In embodiments, the modified Ras mutant protein comprises a compound described herein covalently bonded to the amino acid residue corresponding to position 12 or 13 of SEQ ID No: 4, wherein the Ras mutant protein is a mammalian protein (including human protein) selected from HRas G12D, HRas G12C, HRas G12S, HRas G13D, HRas G13C, and HRas G13S. It will be understood that a compound described herein may be modified upon covalently binding an amino acid (e.g., mutant amino acid other than G) corresponding to position 12 or 13 of human KRas (e.g., SEQ ID. No: 2). A subject compound of the present disclosure encompasses a compound described herein immediately prior to covalently bonding the Ras mutant protein as well as the resulting compound covalently bonded to the modified Ras mutant protein.

In embodiments, the modified Ras mutant protein described herein is formed by contacting a compound described herein with the serine residue of an unmodified Ras G12S mutant protein, wherein the compound comprises a moiety susceptible to reacting with a nucleophilic serine residue corresponding to position 12 of SEQ ID No: 1. In some embodiments, the compound comprises a staying group and a leaving group, wherein said contacting results in release of the leaving group and formation of said modified protein. In some embodiments, the compound selectively labels the serine residue corresponding to position 12 of SEQ ID No. 1 (a G12S mutant) relative to a valine (G12V) residue at the same position. In some embodiments, the compound selectively labels the serine residue as compared to (i) an aspartate residue of a K-Ras G12D mutant protein, said aspartate corresponding to residue 12 of SEQ ID NO: 7, and/or (ii) a valine residue of a K-Ras G12V mutant protein, said valine corresponding to residue 12 of SEQ ID NO: 8. In some embodiments, the compound selectively labels the serine residue as compared to (i) an aspartate residue of a K-Ras G12D mutant protein, said aspartate corresponding to residue 12 of SEQ ID NO: 7, and/or (ii) a valine residue of a K-Ras G12V mutant protein, said valine corresponding to residue 12 of SEQ ID NO: 8, by at least 1, 2, 3, 4, 5, 10 folds or more, when assayed under comparable conditions. In some embodiments, the compound selectively labels the serine residue corresponding to position 12 of SEQ ID No. 1 (a G12S KRas mutant) relative to a glycine residue at the same position in wildtype KRas.

In some aspects, a subject compound exhibits one or more of the following characteristics: it is capable of reacting with a mutant residue (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C or HRas G13S) of a Ras mutant protein and covalently modify such Ras mutant and/or it comprises a moiety susceptible to reacting with a nucleophilic amino acid residue corresponding to position 12 or 13 of SEQ ID No: 2 (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S). In some embodiments, a subject compound when used to modify a Ras mutant protein, reduces the Ras protein's signaling output. In some embodiments, a subject compound exhibits an IC50 (against a mutant Ras (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S), as ascertained by reduction of Ras::SOS1 interaction) of less than 10 uM, 5 uM, 1 uM, 500 nM, less than 100 nM, less than 50 nM, 10 nM, 5 nM, 1 nM, 500 pM, 50 pM, 10 pM or less. In some embodiments, a subject compound exhibits an IC50 (against a mutant Ras (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S), as ascertained by an assay described herein) of less than 10 uM, 5 uM, 1 uM, 500 nM, less than 100 nM, less than 50 nM, 10 nM, 5 nM, 1 nM, 500 pM, 50 pM, 10 pM or less.

In practicing any of the methods disclosed herein, the Ras target to which a subject compound binds covalently can be a Ras mutant (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S).

In an aspect is provided a method of inhibiting cell growth, comprising administering an effective amount of a compound described herein, or a pharmaceutically acceptable salt or solvate thereof, to a cell expressing a Ras protein, thereby inhibiting growth of said cells.

In embodiments, the method includes administering an additional agent.

In embodiments, the cancer is a solid tumor.

In embodiments, the cancer is a hematological cancer.

In some embodiments, the methods of treating cancer can be applied to treat a solid tumor or a hematological cancer. In some embodiments, the cancer being treated can be, without limitation, prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein, or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

Any of the treatment methods disclosed herein can be administered alone or in combination or in conjunction with another therapy or another agent. By “combination” it is meant to include (a) formulating a subject composition containing a subject compound together with another agent, and (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiment, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof.

In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/M4X-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, RevimmuneT), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In an aspect, compositions provided herein can be administered in combination with radiotherapy such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-11BB antibody (e.g., urelumab, PF-05082566), an anti-OX40 antibody (pogalizumab tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody. In some embodiments, the checkpoint immune blockade agents are selected from the group consisting of anti-PD-L1 antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7W, 410C9), anti-B7-113 antibodies (e.g., DS-5573a), anti-TIM3 antibodies (e.g., F38-2E2), and combinations thereof. In one embodiment, the checkpoint immune blockade agent is an anti-PD-L1 antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldeskeukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In some embodiments, any of the compounds herein that is capable of binding a Ras protein (e.g., KRAS) to modulate activity of such Ras protein may be administered in combination or in conjunction with one or more pharmacologically active agents comprising (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of SHP pathway including SHP2 inhibitor (e.g., 6-(4-amino-4-methylpiperidin-1-yl)-3-(2,3-dichlorophenyl)pyrazin-2-amine, as well as SHP1 inhibitors, or (33) an inhibitor of Src, FLT3, HDAC, VEGFR, PDGFR, LCK, Bcr-Abl or AKT or (34) an inhibitor of KrasG12C mutant (e.g., including but not limited to AMG510, MRTX849, and any covalent inhibitors binding to the cysteine residue 12 of Kras, the structures of these compounds are publicly known)(e.g., an inhibitor of Ras G12C as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety),), (35) a SHC inhibitor (e.g., PP2, AID371185), (36) a GAB inhibitor (e.g., GAB-0001), (37) a GRB inhibitor, (38) a PI-3 kinase inhibitor (e.g., Idelalisib, Copanlisib, Duvelisib, Alpelisib, Taselisib, Perifosine, Buparlisib, Umbralisib, NVP-BEZ235-AN), (39) a MARPK inhibitor, (40) CDK4/6 (e.g., palbociclib, ribociclib, abemaciclib), or (41) MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197), or (42) an inhibitor of SHP pathway including SHP2 inhibitor (e.g., 6-(4-amino-4-methylpiperidin-1-yl)-3-(2,3-dichlorophenyl)pyrazin-2-amine, RMC-4630, ERAS-601,

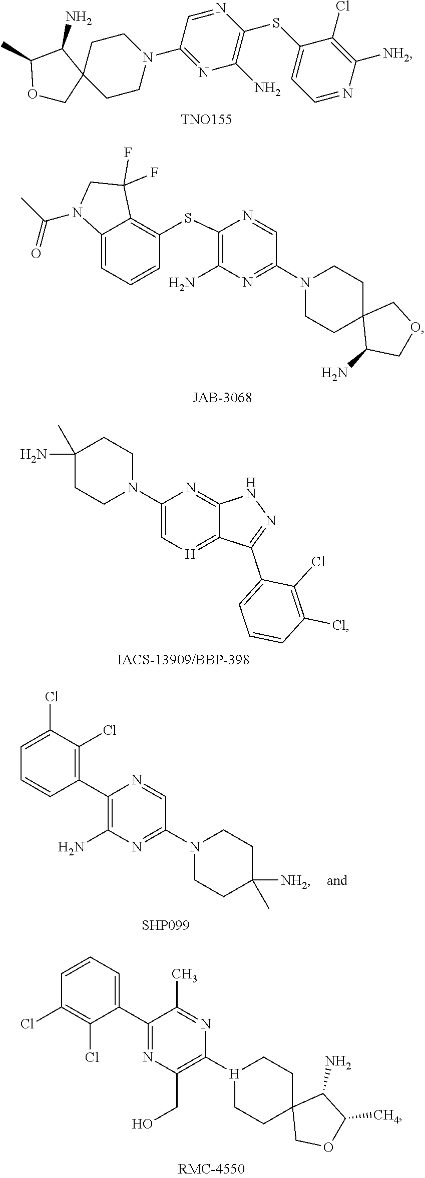

as well as SHP1 inhibitors. In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L1 antibody, anti-CLTA-4 antibody). In some embodiments, any of the compounds herein that is capable of binding a Ras protein (e.g., KRAS) to modulate activity of such Ras protein may be administered in combination or in conjunction with one or more pharmacologically active agents comprising an inhibitor against one or more targets selected from the group of: MEK, epidermal growth factor receptor (EGFR), FGFR1, FGFR2, FGFR3, mitotic kinase, topoisomerase, ALK, c-MET, ErbB2, AXL, NTRK1, RET, A-Raf, B-Raf, C-Raf, ERK, MDM2, mTOR, BET, IGF1/2, IGF1-R, CDK9, SHIP1, SHIP2, SHP2, SRC, JAK, PARP, BTK, FLT3, HDAC, VEGFR, PDGFR, LCK, Bcr-Abl, AKT, KrasG12C mutant, and ROS1. Where desired, the additional agent can be an inhibitor against one or more targets selected from the group of: MEK, epidermal growth factor receptor (EGFR), FGFR1, FGFR2, FGFR3, mitotic kinase, topoisomerase, ALK, c-MET, ErbB2, AXL, NTRK1, RET, A-Raf, B-Raf, C-Raf, ERK, MDM2, mTOR, BET, IGF1/2, IGF1-R, CDK9, SHP2, SRC, JAK, PARP, BTK, FLT3, HDAC, VEGFR, PDGFR, LCK, Bcr-Abl, AKT, KrasG12C mutant, and ROS1. In some embodiments, any of the compounds herein that is capable of binding a Ras protein (e.g., KRAS, mutant Ras protein) to modulate activity of such Ras protein (e.g., mutant Ras protein such as G12D or G12S mutant KRas protein) may be administered in combination or in conjunction with one or more additional pharmacologically active agents comprising an inhibitor of SOS (e.g., SOS1, SOS2) or of mutants thereof. In embodiments, the additional pharmacologically active agent administered in combination or in conjunction with a compound described herein (e.g., compound capable of binding a Ras protein) is an inhibitor of SOS (e.g., SOS1, SOS2). In embodiments, the additional pharmacologically active agent administered in combination or in conjunction with a compound (e.g., compound capable of binding a Ras protein) described herein is an inhibitor of SOS (e.g., SOS1, SOS2). In embodiments, the additional pharmacologically active agent administered in combination or in conjunction with a compound (e.g., compound capable of binding a Ras protein) described herein is an inhibitor of SOS (e.g., SOS1, SOS2) selected from

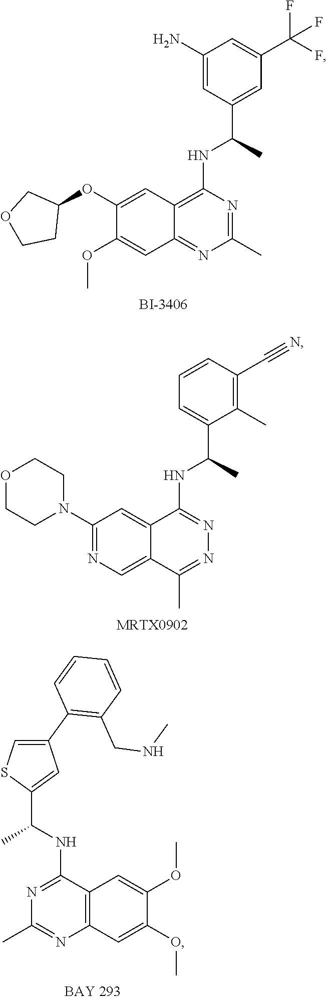

RMC-5845, and BI-1701963. In embodiments, the additional pharmacologically active agent administered in combination or in conjunction with a compound described herein (e.g., compound capable of binding a Ras protein) is an inhibitor of SOS (e.g., SOS1, SOS2) described in WO2021092115, WO2018172250, WO2019201848, WO2019122129, WO2018115380, WO2021127429, WO2020180768, or WO2020180770, all of which are herein incorporated by reference in their entirety for all purposes.

In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L1 antibody, anti-CLTA-4 antibody).

In some embodiments, any of the compounds described herein that is capable of binding a Ras protein (e.g., KRAS) may be administered in combination or in conjunction with one or more pharmacologically active agents comprising an inhibitor of: (1) SOS1 or a mutant thereof (e.g., RMC-5845, BI-3406, BAY-293, BI-1701963); (2) SHP2 or a mutant thereof (e.g., 6-(4-amino-4-methylpiperidin-1-yl)-3-(2,3-dichlorophenyl)pyrazin-2-amine, TNO155, RMC-4630, ERAS-601, JAB-3068, IACS-13909/BBP-398, SHP099, RMC-4550); (3) SHC or a mutant thereof (e.g., PP2, AID371185); (4) GAB or a mutant thereof (e.g., GAB-0001); (5) GRB or a mutant thereof; (6) JAK or a mutant thereof (e.g., tofacitinib); (7) A-RAF, B-RAF, C-RAF, or a mutant thereof (e.g., RAF-709, LY-3009120); (8) BRAF or a mutant thereof (e.g., Sorafenib, Vemurafenib, Dabrafenib, Encorafenib, regorafenib, GDC-879); (9) MEK or a mutant thereof (e.g., trametinib, cobimetinib, binimetinib, selumetinib, refametinib, AZD6244); (10) ERK or a mutant thereof (e.g., ulixertinib, MK-8353, LTT462, AZD0364, SCH772984, BIX02189, LY3214996, ravoxertinib); (11) PI3K or a mutant thereof (e.g., Idelalisib, Copanlisib, Duvelisib, Alpelisib, Taselisib, Perifosine, Buparlisib, Umbralisib, NVP-BEZ235-AN); (12) MAPK or a mutant thereof (e.g., VX-745, VX-702, RO-4402257, SCIO-469, BIRB-796, SD-0006, PH-797804, AMG-548, LY2228820, SB-681323, GW-856553, RWJ67657, BCT-197); (13) EGFR or a mutant thereof (e.g., afatinib, erlotinib, gefitinib, lapatinib, cetuximab panitumumab, osimertinib, olmutinib, EGF-816); (14) c-MET or a mutant thereof (e.g., K252a, SU11274, PHA665752, PF2341066); (15) ALK or a mutant thereof (e.g. crizotinib, alectinib, entrectinib, brigatinib); (16) FGFR1, FGFR-2, FGFR-3, FGFR-4 or a mutant thereof (e.g., nintedanib); (17) BCR-ABL or a mutant thereof (e.g., imatinib, dasatinib, nilotinib); (18) ErbB2 (Her2) or a mutant thereof (e.g., afatinib, lapatinib, trastuzumab, pertuzumab); (19) AXL or a mutant thereof (e.g., R428, amuvatinib, XL-880); (20) NTRK1 or a mutant thereof (e.g., merestinib); (21) ROS1 or a mutant thereof (e.g., entrectinib); (22) RET or a mutant thereof (e.g., BLU-667, Lenvatinib); (23) MDM2 or a mutant thereof (e.g., HDM-201, NVP-CGM097, RG-71 12, MK-8242, RG-7388, SAR405838, AMG-232, DS-3032, RG-7775, APG-115); (24) mTOR or a mutant thereof (e.g., rapamycin, temsirolimus, everolimus, ridaforolimus); (25) BET or a mutant thereof (e.g., I-BET 151, I-BET 762, OTX-015, TEN-010, CPI-203, CPI-0610, olionon, RVX-208, ABBC-744, LY294002, AZD5153, MT-1, MS645); (26) IGF1, IGF2, IGF1R, or a mutant thereof (e.g., xentuzumab, MEDI-573); (27) CDK9 or a mutant thereof (e.g., DRB, flavopiridol, CR8, AZD 5438, purvalanol B, AT7519, dinaciclib, SNS-032); or (28) CDK4/6 (e.g., palbociclib, ribociclib, abemaciclib).

In combination therapy, a compound provided herein and other anti-cancer agent(s) may be administered either simultaneously, concurrently or sequentially with no specific time limits, wherein such administration provides therapeutically effective levels of the two compounds in the body of the patient.

In some embodiments, the compound of the present disclosure and the other anti-cancer agent(s) are generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

An antibiotic can be administered to a subject as part of a therapeutic regime. An antibiotic can be administered at a therapeutically effective dose. An antibiotic can kill or inhibit growth of bacteria. An antibiotic can be a broad spectrum antibiotic that can target a wide range of bacteria. Broad spectrum antibiotics, either a 3^{rd} or 4^{th} generation, can be cephalosporin or a quinolone. An antibiotic can also be a narrow spectrum antibiotic that can target specific types of bacteria. An antibiotic can target a bacterial cell wall such as penicillins and cephalosporins. An antibiotic can target a cellular membrane such as polymyxins. An antibiotic can interfere with essential bacterial enzymes such as antibiotics: rifamycins, lipiarmycins, quinolones, and sulfonamides. An antibiotic can also be a protein synthesis inhibitor such as macrolides, lincosamides, and tetracyclines. An antibiotic can also be a cyclic lipopeptide such as daptomycin, glycylcyclines such as tigecycline, oxazolidiones such as linezolid, and lipiarmycins such as fidaxomicin. In some cases, an antibiotic can be 1^{st} generation, 2^{nd} generation, 3^{rd} generation, 4^{th} generation, or 5^{th} generation. A first-generation antibiotic can have a narrow spectrum. Examples of 1^{st} generation antibiotics can be penicillins (Penicillin G or Penicillin V), Cephalosporins (Cephazolin, Cephalothin, Cephapirin, Cephalethin, Cephradin, or Cephadroxin). In some cases, an antibiotic can be 2^{nd} generation. 2^{nd} generation antibiotics can be a penicillin (Amoxicillin or Ampicillin), Cephalosporin (Cefuroxime, Cephamandole, Cephoxitin, Cephaclor, Cephrozil, Loracarbef). In some cases, an antibiotic can be 3^{rd} generation. A 3_{rd} generation antibiotic can be penicillin (carbenicillin and ticarcillin) or cephalosporin (Cephixime, Cephtriaxone, Cephotaxime, Cephtizoxime, and Cephtazidime). An antibiotic can also be a 4^{th} generation antibiotic. A 4^{th} generation antibiotic can be Cephipime. An antibiotic can also be 5^{th} generation. 5^{th} generation antibiotics can be Cephtaroline or Cephtobiprole.

In some cases, an anti-viral agent may be administered as part of a treatment regime. In some cases, a herpes virus prophylaxis can be administered to a subject as part of a treatment regime. A herpes virus prophylaxis can be valacyclovir (Valtrex). Valtrex can be used orally to prevent the occurrence of herpes virus infections in subjects with positive HSV serology. It can be supplied in 500 mg tablets. Valacyclovir can be administered at a therapeutically effective amount.

In some cases, a treatment regime may be dosed according to a body weight of a subject. In subjects who are determined obese (BMI >35) a practical weight may need to be utilized. BMI is calculated by: BMI=weight (kg)/[height (m)]².

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases, a body surface area may be utilized to calculate a dosage. A body surface area (BSA) may be calculated by: BSA (m2)=√Height (cm)*Weight (kg)/3600.

In an aspect is provided a method of modulating activity of a Ras (e.g., K-Ras) protein, comprising contacting a Ras protein with an effective amount of a compound described herein, or a pharmaceutically acceptable salt or solvate thereof, thereby modulating the activity of the Ras (e.g., K-Ras) protein.

In some embodiments, the subject method comprises administering an additional agent or therapy.

In some embodiments is a method of modulating activity of a Ras protein, comprising contacting a Ras protein with an effective amount of a compound described, or a pharmaceutically acceptable salt or solvate thereof, wherein said modulating comprises inhibiting the Ras (e.g., K-Ras) protein activity. In some embodiments is a method of modulating activity of a Ras protein including Ras G12S mutant proteins such as K-Ras G12S, H-Ras G12S, and N-Ras G12S, comprising contacting the Ras protein with an effective amount of a compound described herein, or a pharmaceutically acceptable salt or solvate thereof.

In some embodiments, provided is a method of reducing Ras signaling output in a cell by contacting the cell with a compound described herein. A reduction in Ras signalling can be evidenced by one or more members of the following: (i) an increase in steady state level of GDP-bound modified protein or a decrease in steady state level of GTP-bound modified protein; (ii) a reduction of phosphorylated AKTs473, (iii) a reduction of phosphorylated ERKT202/y204, (iv) a reduction of phosphorylated S6S235/236, and (v) reduction of cell growth of a tumor cell expressing a Ras G12S mutant protein, and (vi) reduction in Ras interaction with a Ras-pathway signaling protein. Non-limiting examples of Ras-pathway signaling protein include SOS (including SOS1 and SOS2), RAF, SHC, SHP (including SHP1 and SHP2), MEK, MAPK, ERK, GRB, RASA1, and GNAQ. In some cases, the reduction in Ras signaling output can be evidenced by two, three, four or all of (i)-(v) above. In some embodiments, the reduction any one or more of (i)-(v) can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100- fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to control untreated with a subject compound. A reduction in cell growth can be demonstrated with the use of tumor cells or cell lines. A tumor cell line can be derived from a tumor in one or more tissues, e.g., pancreas, lung, ovary, biliary tract, intestine (e.g., small intestine, large intestine (i.e. colon)), endometrium, stomach, hematopoietic tissue (e.g., lymphoid tissue), etc. Examples of the tumor cell line with a K-Ras mutation may include, but are not limited to, A549 (e.g., K-Ras G12S), AGS (e.g., K-Ras G12D), ASPC1 (e.g., K-Ras G12D), Calu-6 (e.g., K-Ras Q61K), CFPAC-1 (e.g., K-Ras G12V), CL40 (e.g., K-Ras G12D), COL0678 (e.g., K-Ras G12D), COR-L23 (e.g., K-Ras G12V), DAN-G (e.g., K-Ras G12V), GP2D (e.g., K-Ras G12D), GSU (e.g., K-Ras G12F), HCT116 (e.g., K-Ras G13D), HEC1A (e.g., K-Ras G12D), HEC1B (e.g., K-Ras G12F), HEC50B (e.g., K-Ras G12F), HEYA8 (e.g., K-Ras G12D or G13D), HPAC (e.g., K-Ras G12D), HPAFII (e.g., K-Ras G12D), HUCCT1 (e.g., K-Ras G12D), KARPAS620 (e.g., K-Ras G13D), KOPN8 (e.g., K-Ras G13D), KP-3 (e.g., K-Ras G12V), KP-4 (e.g., K-Ras G12D), L3.3 (e.g., K-Ras G12D), LoVo (e.g., K-Ras G13D), LS180 (e.g., K-Ras G12D), LS513 (e.g., K-Ras G12D), MCAS (e.g., K-Ras G12D), NB4 (e.g., K-Ras A18D), NCI-H1355 (e.g., K-Ras G13C), NCI-H1573 (e.g., K-Ras G12A), NCI-H1944 (e.g., K-Ras G13D), NCI-H2009 (e.g., K-Ras G12A), NCI-H441 (e.g., K-Ras G12V), NCI-H747 (e.g., K-Ras G13D), NOMO-1 (e.g., K-Ras G12D), OV7 (e.g., K-Ras G12D), PANC0203 (e.g., K-Ras G12D), PANC0403 (e.g., K-Ras G12D), PANC0504 (e.g., K-Ras G12D), PANC0813 (e.g., K-Ras G12D), PANC1 (e.g., K-Ras G12D), Panc-10.05 (e.g., K-Ras G12D), PaTu-8902 (e.g., K-Ras G12V), PK1 (e.g., K-Ras G12D), PK45H (e.g., K-Ras G12D), PK59 (e.g., K-Ras G12D), SK-CO-1 (e.g., K-Ras G12V), SKLU1 (e.g., K-Ras G12D), SKM-1 (e.g., K-Ras K117N), SNU1 (e.g., K-Ras G12D), SNU1033 (e.g., K-Ras G12D), SNU1197 (e.g., K-Ras G12D), SNU407 (e.g., K-Ras G12D), SNU410 (e.g., K-Ras G12D), SNU601 (e.g., K-Ras G12D), SNU61 (e.g., K-Ras G12D), SNU8 (e.g., K-Ras G12D), SNU869 (e.g., K-Ras G12D), SNU-C2A (e.g., K-Ras G12D), SU.86.86 (e.g., K-Ras G12D), SUIT2 (e.g., K-Ras G12D), SW1990 (e.g., K-Ras G12D), SW403 (e.g., K-Ras G12V), SW480 (e.g., K-Ras G12V), SW620 (e.g., K-Ras G12V), SW948 (e.g., K-Ras Q⁶¹L), T₃M10 (e.g., K-Ras G12D), TCC-PAN2 (e.g., K-Ras G12R), TGBC11TKB (e.g., K-Ras G12D), and MIA Pa-Ca (e.g., MIA Pa-Ca 2 (e.g., K-Ras G12C)).

In some embodiments, the compounds of the present invention exhibit one or more functional characteristics disclosed herein. For example, a subject compound binds to a Ras protein, Kras protein or a mutant form thereof. In some embodiments, a subject compound binds specifically and also inhibits a Ras protein, Kras protein or a mutant form thereof. In some embodiments, a subject compound selectively inhibits a Kras mutant relative to a wildtype Kras. In some embodiments, a subject compound selectively inhibits KrasG12D and/or KrasG12V relative to wildtype Kras. In some embodiments, the IC50 of a subject compound for a Kras mutant (e.g., including G12D) is less than about 5 pM, less than about 1 pM, less than about 500 nM, less than 100 nM or less than 10 nM, as measured in an in vitro assay known in the art or exemplified herein. In some embodiments, a subject compound selectively inhibits KrasG12S and/or KrasG12C relative to wildtype Kras or KrasG12D. In some embodiments, the IC50 of a subject compound for a Kras mutant (e.g., including G12S) is less than about 5 pM, less than about 1 pM, less than about 500 nM, or less than about 100 nM, as measured in an in vitro assay known in the art or exemplified herein.

In some embodiments, a subject compound of the present disclosure is capable of reducing Ras signaling output. Such reduction can be evidenced by one or more members of the following: (i) an increase in steady state level of GDP-bound Ras protein or a decrease in steady state level of GTP-bound modified protein; (ii) a reduction of phosphorylated AKTs473, (iii) a reduction of phosphorylated ERKT202/y204, (iv) a reduction of phosphorylated S6S235/236, and (v) reduction (e.g., inhibition) of cell growth of Ras-driven tumor cells (e.g., those derived from a tumor cell line disclosed herein). In some cases, the reduction in Ras signaling output can be evidenced by two, three, four or all of (i)-(v) above.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

### Pharmaceutical Compositions and Methods of Administration

In an aspect is provided a pharmaceutical composition comprising a compound described herein, or a pharmaceutically acceptable salt or solvate thereof, and a pharmaceutically acceptable excipient.

The compounds described herein, or a pharmaceutically acceptable salt or solvate thereof, are administered to subjects in a biologically compatible form suitable for administration to treat or prevent diseases, disorders or conditions. Administration of the compounds described herein can be in any pharmacological form including a therapeutically effective amount of a compound described herein, or a pharmaceutically acceptable salt or solvate thereof, alone or in combination with a pharmaceutically acceptable carrier.

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments of the methods described herein, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments of the methods described herein, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### Additional Embodiments

Embodiment 1: A compound of Formula (I), or a pharmaceutically acceptable salt or solvate thereof:

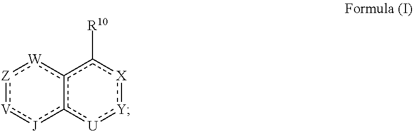

wherein
W is a N, C(R⁸), N(R^{18b}), C(R¹¹)(R^{18a}), C(O), S(O), or S(O)₂;
Z is N, C(R⁸), N(R^{8b}), C(R⁸)(R^{a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{6a}), N, N(R^{16a}), and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), or S(O)₂;
U is N, C(R²), C(R²)(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is

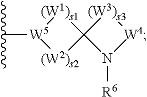

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵ and wherein R⁶ is optionally capable of forming a covalent bond with a Ras amino acid;
L² is a bond, —O—, —N(R^{4d})—, —C(O)—, —S—, —S(O)₂—, —S(O)—, —P(O)R^{4d}—, CR^{4c}R^{4c}, —OCR^{4c}R^{4c}—, —N(R^{4d})CR^{4c}R^{4c}—, —C(O)CR^{4c}R^{4c}—, —SCR^{4c}R^{4c}—, —S(O)₂CR^{4c}R^{4c}—, —S(O)CR^{4c}R^{4c}—, —P(O)R^{4d}CR^{4c}R^{4c}—, —CR^{4c}R^{4c}R^{4c}R^{4c}, —CR^{4c}R^{4c}O—, —CR^{4c}R^{4c}N(R^{4d})—, —CR^{4c}R^{4c}C(O)—, —CR^{4c}R^{4c}S—, —CR^{4c}R^{4c}S(O)₂—, —CR^{4c}R^{4c}S(O)—, —CR^{4c}R^{4c}P(O)R^{4d}—, —N(R^{4d})C(O)—, —N(R^{4d})S(O)₂—, —N(R^{4d})S(O)—, —N(R^{4d})P(O)R^{4d}—, —C(O)N(R^{4d})—, —S(O)₂N(R^{4d})—, —S(O)N(R^{4d})—, —P(O)R^{4d}N(R^{4d})—, —OC(O)—, —OS(O)₂—, —OS(O)—, —OP(O)R^{4d}—, —C(O)O—, —S(O)₂O—, —S(O)O—, —P(O)R^{4d}O—, —CR^{4c}R^{4c}R^{4c}R^{4c}CR^{4c}R^{4c}—, —OCR^{4c}R^{4c}CR^{4c}R^{4c}—, —N(R^{4d})CR^{4c}R^{4c}R^{4c}R^{4c}, C(O)CR^{4c}R^{4c}CR^{4c}R^{4c}—, —SCR^{4c}R^{4c}R^{4c}R^{4c}—, —S(O)₂CR^{4c}R^{4c}CR^{4c}R^{4c}—, —S(O)CR^{4c}R^{4c}CR^{4c}R^{4c}—, —P(O)R^{4d}CR^{4c}R^{4c}R^{4c}R^{4c}—, —CR^{4c}R^{4c}R^{4c}O, —CR^{4c}R^{4c}N(R^{4d}), —R^{4c}R^{4c}C(O)—, —CR^{4c}R^{4c}CR^{4c}R^{4c}S—, —CR^{4c}R^{4c}R^{4c}R^{4c}S(O)₂—, —CR^{4c}R^{4c}R^{4c}R^{4c}S(O)—, or —CR^{4c}R^{4c}R^{4c}P(O)R^{4d}—;
each R^{4c} is independently selected from hydrogen, halogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, —OC(O)R^{14a}, —N(R¹⁴)(R¹⁴), —N(R¹⁴)C(O)N(R¹⁴)(R¹⁴), —N(R¹⁴)C(O)OR¹⁴, —N(R¹⁴)C(O)R^{14a}, and —N(R¹⁴)S(O)₂R¹⁴, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
each R^{1d} is independently selected from hydrogen, —CN, C₁₋₆alkyl, C₂₋₆alkenyl, C₂₋₆alkynyl, C₁₋₆haloalkyl, C₁₋₆alkoxy, C₁₋₆ haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, and —OC(O)R^{14a}, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_g S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²¹;
R¹⁷ is -L¹-R¹⁹;
R^{17b} is -L b-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R^{1f})(R^{1g})O, C(R^{1f})(R^{1g})N(R^{1c}), and C(R¹)(R^{g}); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2e} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁i-haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f}, R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R²⁰, R^{20d}, R^{20e}, R^{20f}, R^{20g}, R^{20h}, R²⁰ⁱ, R^{20k}, and R²⁰mare each independently selected from halogen, oxo, —CN, C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each Ru is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.
   Embodiment 2: The compound of embodiment 1, or a pharmaceutically acceptable salt or solvate thereof, wherein R⁶ is capable of forming a covalent bond with a Ras amino acid sidechain.
   Embodiment 3: The compound of embodiment 2, or a pharmaceutically acceptable salt or solvate thereof, wherein R⁶ is capable of forming a covalent bond with a KRas amino acid.
   Embodiment 4: The compound of embodiment 2, or a pharmaceutically acceptable salt or solvate thereof, wherein R⁶ is capable of forming a covalent bond with the 121 amino acid of a human KRas protein.
   Embodiment 5: The compound of embodiment 2, or a pharmaceutically acceptable salt or solvate thereof, wherein R⁶ is capable of forming a covalent bond with the 121 amino acid of a mutant KRas protein selected from KRas G12D, KRas G12C, and KRas G12S.
   Embodiment 6: The compound of embodiment 2, or a pharmaceutically acceptable salt or solvate thereof, wherein R⁶ is capable of forming a covalent bond with the 13& amino acid of a human KRas protein.
   Embodiment 7: The compound of embodiment 2, or a pharmaceutically acceptable salt or solvate thereof, wherein R⁶ is capable of forming a covalent bond with the 13& amino acid of a mutant KRas protein selected from KRas G13D, KRas G13C, and KRas G13S.
   Embodiment 8: The compound of any one of embodiments 2-7, or a pharmaceutically acceptable salt or solvate thereof, wherein R⁶ is selected from the group consisting of

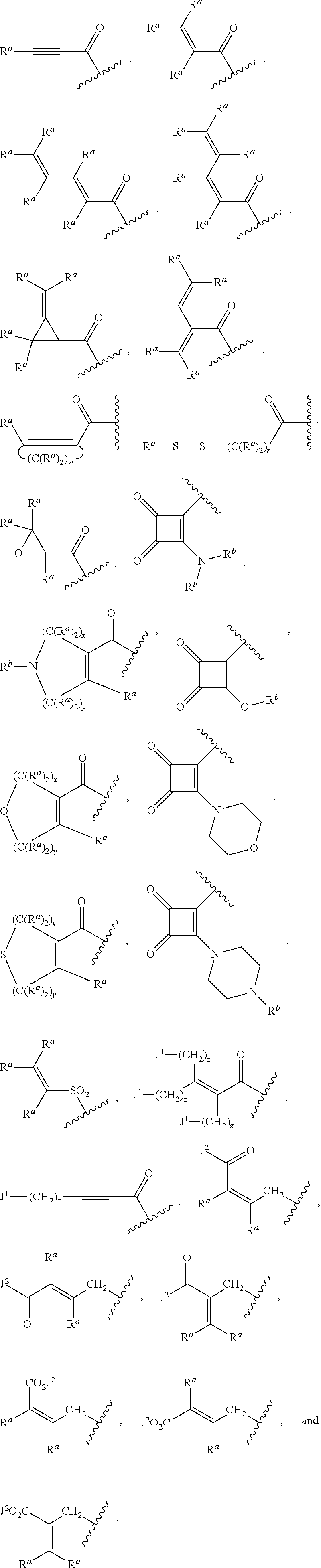

where each R^{a} is independently hydrogen, C₁₋₆alkyl, carboxy, C₁₋₆carboalkoxy, phenyl, C₂₋₇carboalkyl, R^{1c}—(C(R^{b})₂)ᵣ—, R^{1c}—(C(R^{b})₂)_{w}-M-(C(R^{b})₂)ᵣ, (R^{d})(R^{e})CH-M-(C(R^{b})₂)ᵣ, or Het-J³-(C(R^{b})₂)ᵣ; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; each R^{e} is independently —NR^{b}R^{b} or —OR^{b}; R^{d} and R^{e} are each, independently, —(C(R^{b})₂)ᵣNR^{b}R^{b}, or —(C(R^{b})₂)ᵣ—OR^{b}; each J² is independently hydrogen, chlorine, fluorine, or bromine; J² is C₁₋₆alkyl or hydrogen; each M is independently —N(R^{b})—, —O—, —N[(C(R^{b})₂)_{w}—NR^{b}R^{b}]—, or —N[(C(R^{b})₂)_{w}—OR^{b}]—; each J³ is independently —N(R^{b})—, —O—, or a bond; each Het is independently a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; each r is independently 1-4; each w is independently 2-4; x is 0-1; y is 0-4, and each z is independently 1-6; wherein the sum of x+y is 2-4.
Embodiment 9: The compound of any one of embodiments 2-7, or a pharmaceutically acceptable salt or solvate thereof, wherein R⁶ is selected from the group consisting of

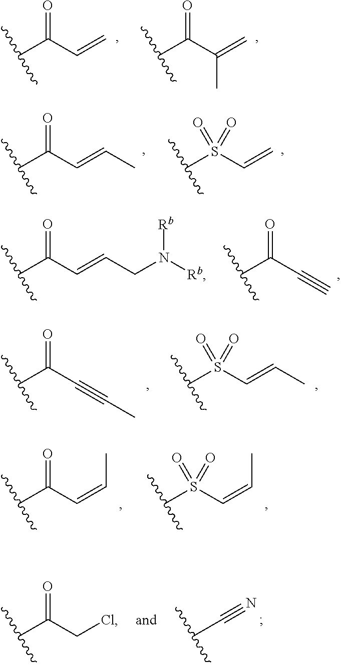

where each R^{b} is independently selected from the group consisting of hydrogen, hydroxyl, C₁-C₆ alkoxy, and C₁-C₆ alkyl.
Embodiment 10: The compound of any one of embodiments 1-7, or a pharmaceutically acceptable salt or solvate thereof, wherein
L² is a bond, —C(O)NH—, —NHC(O)—, or —C(O)—; and
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k}.
   Embodiment 11: A compound of Formula (II), or a pharmaceutically acceptable salt or solvate thereof:

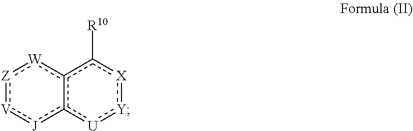

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16b}); wherein exactly one of V and J is C(R¹⁷), C(R¹⁷)(R^{17a}), or N(R¹⁷);
Y is N, C(R²), C(R²)(R^{2c}), N(R^{2b}), S(O), S(O)₂, or C(O);
U is N, C(R^{2c}), C(R^{2c})(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is

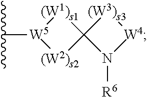

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵ and wherein R⁶ is optionally capable of forming a covalent bond with a Ras amino acid;
L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R¹⁷ is -L¹-R¹⁹;
R^{17b} is -L^{1b}-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R¹)(R¹⁹)O, C(R¹)(R¹⁹)N(R^{1c}), and C(R¹)(R¹⁹); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R¹)(R¹⁹)O, C(R¹)(R¹⁹)N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R¹⁹ are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, three, four, five, six, or seven R¹ⁱ each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2e} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²;
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R²⁰, R^{20d}, R^{20e}, R^{20f}, R^{20g}, R^{20h}, R²⁰ⁱ, R^{20k}, and R²⁰mare each independently selected from halogen, oxo, —CN, C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.
   Embodiment 12: The compound of embodiment 11, or a pharmaceutically acceptable salt or solvate thereof, wherein R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ.
   Embodiment 13: The compound of any one of embodiments 11-12, or a pharmaceutically acceptable salt or solvate thereof, wherein R⁶ is capable of forming a covalent bond with a Ras amino acid.
   Embodiment 14: The compound of any one of embodiments 11-12, or a pharmaceutically acceptable salt or solvate thereof, wherein R⁶ is capable of forming a covalent bond with a KRas amino acid.
   Embodiment 15: The compound of any one of embodiments 11-12, or a pharmaceutically acceptable salt or solvate thereof, wherein R⁶ is capable of forming a covalent bond with the 12^{th} amino acid of a human KRas protein.
   Embodiment 16: The compound of any one of embodiments 11-12, or a pharmaceutically acceptable salt or solvate thereof, wherein R⁶ is capable of forming a covalent bond with the 12^{th} amino acid of a mutant KRas protein selected from KRas G12D, KRas G12C, and KRas G12S.
   Embodiment 17: The compound of any one of embodiments 11-12, or a pharmaceutically acceptable salt or solvate thereof, wherein R⁶ is capable of forming a covalent bond with the 13^{th} amino acid of a human KRas protein.
   Embodiment 18: The compound of any one of embodiments 11-12, or a pharmaceutically acceptable salt or solvate thereof, wherein R⁶ is capable of forming a covalent bond with the 13^{th} amino acid of a mutant KRas protein selected from KRas G13D, KRas G13C, and KRas G13S.
   Embodiment 19: The compound of any one of embodiments 1-18, or a pharmaceutically acceptable salt or solvate thereof, wherein R⁶ is selected from the group consisting of

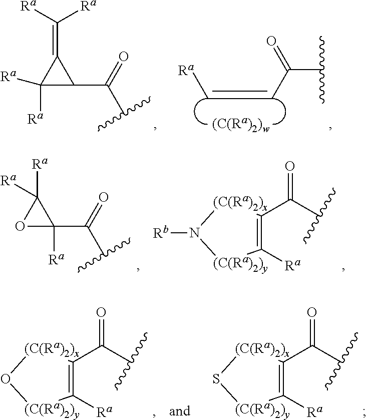

where each R^{a} is independently hydrogen, C₁₋₆alkyl, carboxy, C₁₋₆ carboalkoxy, phenyl, C₂₋₇carboalkyl, R^{1c}—(C(R^{b})₂)ᵣ—, R^{c}—(C(R^{b})₂)_{w}-M-(C(R^{b})₂)ᵣ, (R^{d})(R^{e})CH-M-(C(R^{b})₂)ᵣ, or Het-J³-(C(R^{b})₂)ᵣ; each R^{b} is independently hydrogen, C₁₋₆alkyl, C₂₋₆alkenyl, C₂₋₆alkynyl, C₃₋₆cycloalkyl, C₂₋₇carboalkyl, C₂₋₇carboxyalkyl, phenyl, or phenyl optionally substituted with one or more halogen, C₁₋₆alkoxy, trifluoromethyl, amino, C₁₋₃ alkylamino, C₂₋₆dialkylamino, nitro, azido, halomethyl, C₂₋₇alkoxymethyl, C₂₋₇alkanoyloxymethyl, C₁₋₆alkylthio, hydroxy, carboxyl, C₂₋₇carboalkoxy, phenoxy, phenyl, thiophenoxy, benzoyl, benzyl, phenylamino, benzylamino, C₁₋₆alkanoylamino, or C₁₋₆alkyl; each R^{e} is independently —NR^{b}R^{b} or —OR¹ⁱ; R^{d} and R^{e} are each, independently, —(C(R^{b})₂)ᵣNR^{b}R^{b}, or —(C(R^{b})₂)ᵣ OR^{b}; each J^{P} is independently hydrogen, chlorine, fluorine, or bromine; J² is C₁₋₆alkyl or hydrogen; each M is independently —N(R^{b})—, —O—, —N[(C(R^{b})₂)_{w}—NR^{b}R^{b}]—, or —N[(C(R^{b})₂)_{w}—OR^{b}]—; each J³ is independently —N(R^{b})—, —O—, or a bond; each Het is independently a heterocycle, optionally mono- or di-substituted on carbon or nitrogen with R^{b} and optionally mono-substituted on carbon with —CH₂OR^{b}; wherein the heterocycle is selected from the group consisting of morpholine, thiomorpholine, thiomorpholine S-oxide, thiomorpholine S,S-dioxide, piperidine, pyrrolidine, aziridine, imidazole, 1,2,3-triazole, 1,2,4-triazole, tetrazole, piperazine, tetrahydrofuran, and tetrahydropyran; each r is independently 1-4; each w is independently 2-4; x is 0-1; y is 0-4, and each z is independently 1-6; wherein the sum of x+y is 2-4.
Embodiment 20: The compound of any one of embodiments 1-18, or a pharmaceutically acceptable salt or solvate thereof, wherein R⁵ is a 5 or 6 membered partially unsaturated heterocycloalkyl or a 5 or 6 membered heteroaryl, each optionally substituted with one, two or three R^{2k}, wherein the partially unsaturated 5 or 6 membered heterocycloalkyl or 5 or 6 membered heteroaryl comprises one, two, or three ring nitrogen atoms; and each is bonded to L² through a ring nitrogen.
Embodiment 21: The compound of any one of embodiments 1-18, or a pharmaceutically acceptable salt or solvate thereof, wherein R⁶ is

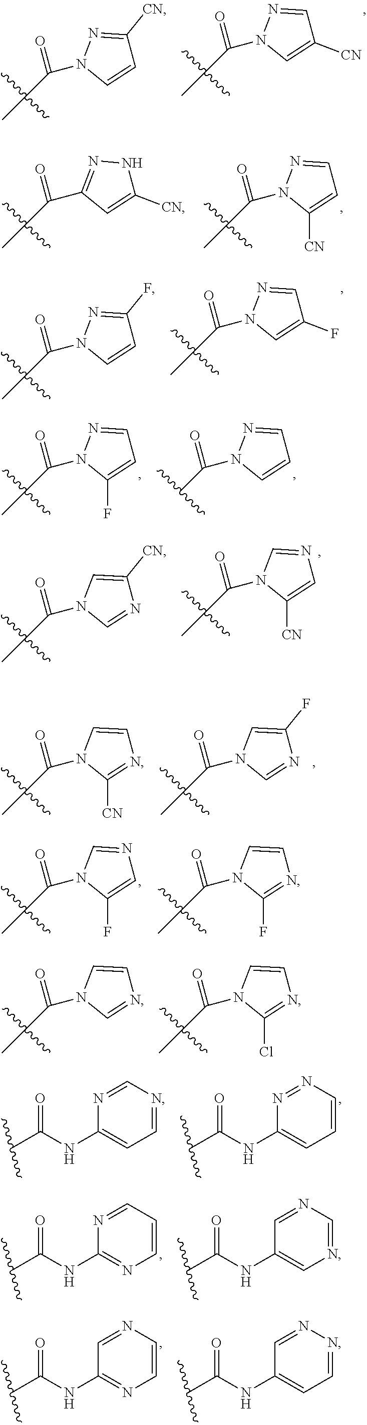

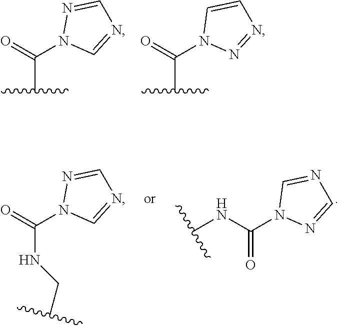

Embodiment 22: The compound of any one of embodiments 1 and 11, or a pharmaceutically acceptable salt or solvate thereof, wherein R⁶ is not capable of forming a covalent bond with the 12^{th} amino acid of a human KRas protein selected from KRas wildtype, KRas G12D, KRas G12C, KRas G12S, KRas G12V, KRas G13D, KRas G13C, KRas G13S, and KRas G13V.
Embodiment 23: The compound of any one of embodiments 1 and 11, or a pharmaceutically acceptable salt or solvate thereof, wherein R⁶ is not capable of forming a covalent bond with the 13^{th} amino acid of human KRas protein selected from KRas wildtype, KRas G12D, KRas G12C, KRas G12S, KRas G12V, KRas G13D, KRas G13C, KRas G13S, and KRas G13V.
Embodiment 24: The compound of any one of embodiments 1 and 11, or a pharmaceutically acceptable salt or solvate thereof, wherein R⁶ is not capable of forming a covalent bond with a KRas amino acid.
Embodiment 25: The compound of any one of embodiments 1 and 11, or a pharmaceutically acceptable salt or solvate thereof, wherein R⁶ is not capable of forming a covalent bond with a Ras amino acid.
Embodiment 26: The compound of any one of embodiments 11 and 22-25, or a pharmaceutically acceptable salt or solvate thereof, wherein
L² is a bond, —C(O)NH—, —NHC(O)—, or —C(O)—; and
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl are optionally substituted with one, two, or three R^{20k}.
   Embodiment 27: The compound of any one of embodiments 11 and 22-25, or a pharmaceutically acceptable salt or solvate thereof, wherein L² is —C(O)—; and R⁵ is a C₃₋₁₂cycloalkyl optionally substituted with one, two or three R^{20k}.
   Embodiment 28: The compound of any one of embodiments 11 and 22-25, or a pharmaceutically acceptable salt or solvate thereof, wherein L² is —C(O)—; and R⁵ is a cyclopropyl optionally substituted with one, two or three R^{20k} selected from halogen and CN.
   Embodiment 29: The compound of any one of embodiments 11 and 22-25, or a pharmaceutically acceptable salt or solvate thereof, wherein R⁶ is

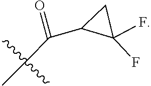 [SMILES: *C(=O)C1CC1(F)F]

Embodiment 30: The compound of any one of embodiments 11 and 22-25, or a pharmaceutically acceptable salt or solvate thereof, wherein R⁶ is

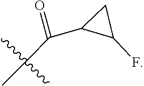 [SMILES: *C(=O)C1CC1F]

Embodiment 31: The compound of any one of embodiments 11 and 22-25, or a pharmaceutically acceptable salt or solvate thereof, wherein R⁶ is

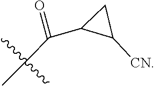 [SMILES: *C(=O)C1CC1C#N]

Embodiment 32: The compound of any one of embodiments 11 and 22-25, or a pharmaceutically acceptable salt or solvate thereof, wherein R⁶ is

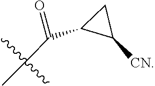 [SMILES: *C(=O)[C@@H]1C[C@H]1C#N]

Embodiment 33: The compound of any one of embodiments 1-7, 10-18, and 22-25, or a pharmaceutically acceptable salt or solvate thereof, wherein
L² is a bond, —C(O)NH—, —NHC(O)—, or —C(O)—;
L² is bonded to a carbon atom of R⁵; and
R⁵ is selected from —CN, C₁₋₆alkyl, C₂₋₆alkenyl, C₂₋₆alkynyl, C₃₋₁₂cycloalkyl, C₆₋₁₂aryl, and C₁₋₁₁heteroaryl, wherein C₁₋₆alkyl, C₂₋₆alkenyl, C₂₋₆alkynyl, C₃₋₁₂cycloalkyl, C₆₋₁₂aryl, and C₁₋₁₁heteroaryl, are optionally substituted with one, two, or three R^{20k}.
   Embodiment 34: The compound of any one of embodiments 1-7, 10-18, and 22-25, or a pharmaceutically acceptable salt or solvate thereof, wherein
L² is —C(O)—;
L² is bonded to a carbon atom of R⁵; and
R⁵ is selected from C₁₋₆alkyl, C₂₋₆alkenyl, C₂₋₆alkynyl, C₃₋₁₂cycloalkyl, C₆₋₁₂aryl, and C₁₋₁₁heteroaryl, wherein C₁₋₆alkyl, C₂₋₆alkenyl, C₂₋₆alkynyl, C₃₋₁₂cycloalkyl, C₆₋₁₂aryl, and C₁₋₁₁heteroaryl, are optionally substituted with one, two, or three R^{20k}.
   Embodiment 35: The compound of any one of embodiments 1-7, 10-18, and 22-25, or a pharmaceutically acceptable salt or solvate thereof, wherein
L² is —C(O)—;
L² is bonded to a carbon atom of R⁵; and
R⁵ is selected from C₁₋₆alkyl, C₂₋₆alkenyl, C₂₋₆alkynyl, C₃₋₁₀cycloalkyl, and 5-6 membered heteroaryl, wherein C₁₋₆alkyl, C₂₋₆alkenyl, C₂₋₆alkynyl, C₃₋₁₀cycloalkyl, and 5-6 membered heteroaryl are optionally substituted with one, two, or three R^{20k}.
   Embodiment 36: The compound of any one of embodiments 1-7, 10-18, and 22-25, or a pharmaceutically acceptable salt or solvate thereof, wherein
L² is —C(O)—;
R⁵ is a heteroaryl having the formula:

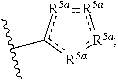

R^{5a} is independently O, S, CH, C(R^{20k}), N, NH, or N(R^{20k});
R⁵ comprises 0-3 independent R^{20k}; and
0-4 R^{5a} are independently N, NH, or N(R^{20k}).
   Embodiment 37: The compound of any one of embodiments 1-7, 10-18, and 22-25, or a pharmaceutically acceptable salt or solvate thereof, wherein
L² is —C(O)—; and
R⁵ is a heteroaryl having the formula:

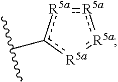

R^{5a} is independently CH, C(R^{20k}), N, NH, or N(R^{20k});
R⁵ comprises 0-3 independent R^{20k}; and
0-4 R^{5a} are independently N, NH, or N(R^{20k}).
   Embodiment 38: The compound of any one of embodiments 1-7, 10-18, and 22-25, or a pharmaceutically acceptable salt or solvate thereof, wherein
L² is —C(O)—;
R⁵ is

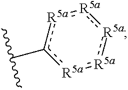

R^{5a} is independently CH, C(R^{20k}), CH(R^{20k}), CH₂, C(R²⁰)₂, N, NH, or N(R^{20k});
R⁵ comprises 0-3 independent R^{20k}; and
0-4 R^{5a} are independently N, NH, or N(R^{20k}).
   Embodiment 39: The compound of any one of embodiments 1-7, 10-18, and 22-25, or a pharmaceutically acceptable salt or solvate thereof, wherein L² is —C(O)—; and R⁵ is C₂₋₆alkenyl, wherein C₂₋₆alkenyl is optionally substituted with one, two, or three R^{20k}.
   Embodiment 40: The compound of any one of embodiments 1-7, 10-18, and 22-25, or a pharmaceutically acceptable salt or solvate thereof, wherein
L² is —C(O)—; and R⁵ is C₂₋₆alkynyl, wherein C₂₋₆alkynyl is optionally substituted with one, two, or three R^{20k}.
   Embodiment 41: A compound of Formula (IV), or a pharmaceutically acceptable salt or solvate thereof:

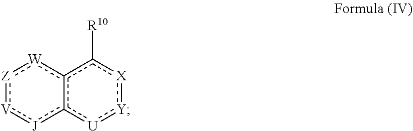

wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}), and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), S(O)₂, or C(O);
U is N, C(R²), C(R²)(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is

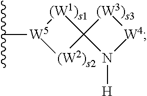

W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6 and s2 is an integer from 2 to 3; or s1 is an integer from 2 to 6 and s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁ heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰;
R¹⁷ is -L¹-R¹⁹;
R^{17b} is -L^{1b}-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1e}), C(O)N(R^{1c}), S(O)₂N(R^{1c}), S(O)N(R^{1c}), C(R^{1f})(R^{1g})O, C(R¹ (R^{1a})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰;
R² is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉ heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12″} is independently selected from C₃₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₃₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
substituted with one, two, or three R^{20h};
each R^{20a}, R^{20b}, R^{20c}, R^{20d}, R^{20e}, R^{20f}, R^{20g}, R^{20h}, R^{2′}, and R^{20m}are each independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉ heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R² is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and
[symbol] indicates a single or double bond such that all valences are satisfied.
   Embodiment 42: The compound of any one of embodiments 11-41, or a pharmaceutically acceptable salt or solvate thereof, wherein Y is C(O).
   Embodiment 43: The compound of any one of embodiments 1-41, or a pharmaceutically acceptable salt or solvate thereof, wherein Y is N.
   Embodiment 44: The compound of any one of embodiments 1-41, or a pharmaceutically acceptable salt or solvate thereof, wherein Y is C(R²).
   Embodiment 45: The compound of any one of embodiments 1-41, or a pharmaceutically acceptable salt or solvate thereof, wherein Y is C(R²)(R^{2c}).
   Embodiment 46: The compound of any one of embodiments 1-41, a pharmaceutically acceptable salt or solvate thereof, wherein Y is S(O).
   Embodiment 47: The compound of any one of embodiments 1-41, or a pharmaceutically acceptable salt or solvate thereof, wherein Y is S(O)₂.
   Embodiment 48: The compound of any one of embodiments 1-47, or a pharmaceutically acceptable salt or solvate thereof, wherein X is N.
   Embodiment 49: The compound of any one of embodiments 1-47, or a pharmaceutically acceptable salt or solvate thereof, wherein X is C(R³).
   Embodiment 50: The compound of any one of embodiments 1-47, or a pharmaceutically acceptable salt or solvate thereof, wherein X is C(R³)(R³).
   Embodiment 51: The compound of any one of embodiments 1-47, or a pharmaceutically acceptable salt or solvate thereof, wherein X is N(R³).
   Embodiment 52: The compound of any one of embodiments 1-51, or a pharmaceutically acceptable salt or solvate thereof, wherein U is N.
   Embodiment 53: The compound of any one of embodiments 1-51, or a pharmaceutically acceptable salt or solvate thereof, wherein U is C(R²).
   Embodiment 54: The compound of any one of embodiments 1-51, or a pharmaceutically acceptable salt or solvate thereof, wherein U is C(R^{2c})(R^{2c}).
   Embodiment 55: The compound of any one of embodiments 1-51, or a pharmaceutically acceptable salt or solvate thereof, wherein U is N(R^{2b}).
   Embodiment 56: The compound of any one of embodiments 1-51, or a pharmaceutically acceptable salt or solvate thereof, wherein U is S(O).
   Embodiment 57: The compound of any one of embodiments 1-51, or a pharmaceutically acceptable salt or solvate thereof, wherein U is S(O)₂.
   Embodiment 58: The compound of any one of embodiments 1-51, or a pharmaceutically acceptable salt or solvate thereof, wherein U is C(O).
   Embodiment 59: The compound of any one of embodiments 1-58, or a pharmaceutically acceptable salt or solvate thereof, wherein W is a N.
   Embodiment 60: The compound of any one of embodiments 1-58, or a pharmaceutically acceptable salt or solvate thereof, wherein W is a C(R¹⁸).
   Embodiment 61: The compound of any one of embodiments 1-58, or a pharmaceutically acceptable salt or solvate thereof, wherein W is a N(R^{8b}).
   Embodiment 62: The compound of any one of embodiments 1-58, or a pharmaceutically acceptable salt or solvate thereof, wherein W is a C(R¹⁸)(R^{18a}).
   Embodiment 63: The compound of any one of embodiments 1-58, or a pharmaceutically acceptable salt or solvate thereof, wherein W is a C(O).
   Embodiment 64: The compound of any one of embodiments 1-58, or a pharmaceutically acceptable salt or solvate thereof, wherein W is a S(O).
   Embodiment 65: The compound of any one of embodiments 1-58, or a pharmaceutically acceptable salt or solvate thereof, wherein W is a S(O)₂.
   Embodiment 66: The compound of any one of embodiments 1-65, or a pharmaceutically acceptable salt or solvate thereof, wherein Z is N.
   Embodiment 67: The compound of any one of embodiments 1-65, or a pharmaceutically acceptable salt or solvate thereof, wherein Z is C(R⁸).
   Embodiment 68: The compound of any one of embodiments 1-65, or a pharmaceutically acceptable salt or solvate thereof, wherein Z is N(R^{8b}).
   Embodiment 69: The compound of any one of embodiments 1-65, or a pharmaceutically acceptable salt or solvate thereof, wherein Z is C(R⁸)(R^{8a}).
   Embodiment 70: The compound of any one of embodiments 1-62, or a pharmaceutically acceptable salt or solvate thereof, wherein Z is C(O).
   Embodiment 71: The compound of any one of embodiments 1-62, or a pharmaceutically acceptable salt or solvate thereof, wherein Z is S(O).
   Embodiment 72: The compound of any one of embodiments 1-62, or a pharmaceutically acceptable salt or solvate thereof, wherein Z is S(O)₂.
   Embodiment 73: The compound of any one of embodiments 1-72, or a pharmaceutically acceptable salt or solvate thereof, wherein V is N(R^{16b}).
   Embodiment 74: The compound of any one of embodiments 1-72, or a pharmaceutically acceptable salt or solvate thereof, wherein V is N.
   Embodiment 75: The compound of any one of embodiments 1-72, or a pharmaceutically acceptable salt or solvate thereof, wherein V is C(R¹⁶)(R^{16a}).
   Embodiment 76: The compound of any one of embodiments 1-72, or a pharmaceutically acceptable salt or solvate thereof, wherein V is C(R¹⁶).
   Embodiment 77: The compound of any one of embodiments 1-72, or a pharmaceutically acceptable salt or solvate thereof, wherein V is N(R^{17b}).
   Embodiment 78: The compound of any one of embodiments 1-72, or a pharmaceutically acceptable salt or solvate thereof, wherein V s C(R¹⁷)(R^{16a}).
   Embodiment 79: The compound of any one of embodiments 1-72, or a pharmaceutically acceptable salt or solvate thereof, wherein V is C(R¹⁷).
   Embodiment 80: The compound of any one of embodiments 1-79, or a pharmaceutically acceptable salt or solvate thereof, wherein J is N(R_{16b}).
   Embodiment 81: The compound of any one of embodiments 1-79, or a pharmaceutically acceptable salt or solvate thereof, wherein J is N.
   Embodiment 82: The compound of any one of embodiments 1-79, or a pharmaceutically acceptable salt or solvate thereof, wherein J is C(R¹⁶)(R^{16a}).
   Embodiment 83: The compound of any one of embodiments 1-79, or a pharmaceutically acceptable salt or solvate thereof, wherein J is C(R¹⁶).
   Embodiment 84: The compound of any one of embodiments 1-76, or a pharmaceutically acceptable salt or solvate thereof, wherein J is N(R^{17b}).
   Embodiment 85: The compound of any one of embodiments 1-76, or a pharmaceutically acceptable salt or solvate thereof, wherein J is C(R¹⁷)(R^{16a}).
   Embodiment 86: The compound of any one of embodiments 1-76, or a pharmaceutically acceptable salt or solvate thereof, wherein J is C(R¹⁷).
   Embodiment 87: The compound of any one of embodiments 1-86, or a pharmaceutically acceptable salt or solvate thereof, wherein L⁷ is a bond.
   Embodiment 88: The compound of any one of embodiments 1-87, or a pharmaceutically acceptable salt or solvate thereof, wherein W¹ and W³ are independently selected from NH, CH₂, C(O), S, O, S(O), and S(O)₂.
   Embodiment 89: The compound of any one of embodiments 1-87, or a pharmaceutically acceptable salt or solvate thereof, wherein W¹ and W³ are independently CH₂.
   Embodiment 90: The compound of any one of embodiments 1-89, or a pharmaceutically acceptable salt or solvate thereof, wherein W² is independently selected from a bond, NH, CH₂, C(O), S, O, S(O), and S(O)₂.
   Embodiment 91: The compound of any one of embodiments 1-89, or a pharmaceutically acceptable salt or solvate thereof, wherein W² is a bond.
   Embodiment 92: The compound of any one of embodiments 1-89, or a pharmaceutically acceptable salt or solvate thereof, wherein W² is CH₂.
   Embodiment 93: The compound of any one of embodiments 1-92, or a pharmaceutically acceptable salt or solvate thereof, wherein W⁴ is CH₂.
   Embodiment 94: The compound of any one of embodiments 1-93, or a pharmaceutically acceptable salt or solvate thereof, wherein W⁵ is N.
   Embodiment 95: The compound of any one of embodiments 1-93, or a pharmaceutically acceptable salt or solvate thereof, wherein W⁵ is CH.
   Embodiment 96: The compound of any one of embodiments 1-95, or a pharmaceutically acceptable salt or solvate thereof, wherein s1 is 1.
   Embodiment 97: The compound of any one of embodiments 1-95, or a pharmaceutically acceptable salt or solvate thereof, wherein s1 is 2.
   Embodiment 98: The compound of any one of embodiments 1-95, or a pharmaceutically acceptable salt or solvate thereof, wherein s1 is 3.
   Embodiment 99: The compound of any one of embodiments 1-95, or a pharmaceutically acceptable salt or solvate thereof, wherein s1 is 4.
   Embodiment 100: The compound of any one of embodiments 1-95, or a pharmaceutically acceptable salt or solvate thereof, wherein s1 is 5.
   Embodiment 101: The compound of any one of embodiments 1-95, or a pharmaceutically acceptable salt or solvate thereof, wherein s1 is 6.
   Embodiment 102: The compound of any one of embodiments 1-101, or a pharmaceutically acceptable salt or solvate thereof, wherein s2 is 1.
   Embodiment 103: The compound of any one of embodiments 1-101, or a pharmaceutically acceptable salt or solvate thereof, wherein s2 is 2.
   Embodiment 104: The compound of any one of embodiments 1-101, or a pharmaceutically acceptable salt or solvate thereof, wherein s2 is 3.
   Embodiment 105: The compound of any one of embodiments 1-104, or a pharmaceutically acceptable salt or solvate thereof, wherein s3 is 1.
   Embodiment 106: The compound of any one of embodiments 1-104, or a pharmaceutically acceptable salt or solvate thereof, wherein s3 is 2.
   Embodiment 107: The compound of any one of embodiments 1-104, or a pharmaceutically acceptable salt or solvate thereof, wherein s3 is 3.
   Embodiment 108: The compound of any one of embodiments 1-107, or a pharmaceutically acceptable salt or solvate thereof, wherein L¹ is a bond and L^{1b} is a bond.
   Embodiment 109: The compound of any one of embodiments 1-108, or a pharmaceutically acceptable salt or solvate thereof, wherein R¹⁹ is selected from a bicyclic C₄₋₁₂cycloalkyl, bicyclic C₂₋₁₁heterocycloalkyl, bicyclic C₇₋₁₂aryl, and bicyclic C₂₋₁₂heteroaryl, wherein the C₄₋₁₂cycloalkyl, bicyclic C₂₋₁₁heterocycloalkyl, bicyclic C₇₋₁₂aryl, and bicyclic C₂₋₁₂heteroaryl are optionally substituted with one, two, three, four, five, six, or seven R¹ⁱ.
   Embodiment 110: The compound of any one of embodiments 1-108, or a pharmaceutically acceptable salt or solvate thereof, wherein R¹⁹ is selected from a bridged bicyclic C₄₋₁₂cycloalkyl, bridged bicyclic C₂₋₁₁heterocycloalkyl, bridged bicyclic C₇₋₁₂aryl, and bridged bicyclic C₂₋₁₂heteroaryl, wherein the bridged bicyclic C₄₋₁₂cycloalkyl, bridged bicyclic C₂₋₁₁heterocycloalkyl, bridged bicyclic C₇₋₁₂aryl, and bridged bicyclic C₂₋₁₂heteroaryl are optionally substituted with one, two, three, four, five, six, or seven R¹ⁱ.
   Embodiment 111: The compound of any one of embodiments 1-108, or a pharmaceutically acceptable salt or solvate thereof, wherein R¹⁹ is selected from a fused bicyclic C₄₋₁₂cycloalkyl, fused bicyclic C₂₋₁₁heterocycloalkyl, fused bicyclic C₇₋₁₂aryl, and fused bicyclic C₂₋₁₂heteroaryl, wherein the fused bicyclic C₄₋₁₂cycloalkyl, fused bicyclic C₂₋₁₁heterocycloalkyl, fused bicyclic C₇₋₁₂aryl, and fused bicyclic C₂₋₁₂heteroaryl are optionally substituted with one, two, three, four, five, six, or seven R¹ⁱ.
   Embodiment 112: The compound of any one of embodiments 1 to 108, or a pharmaceutically acceptable salt or solvate thereof, wherein R¹⁹ is:

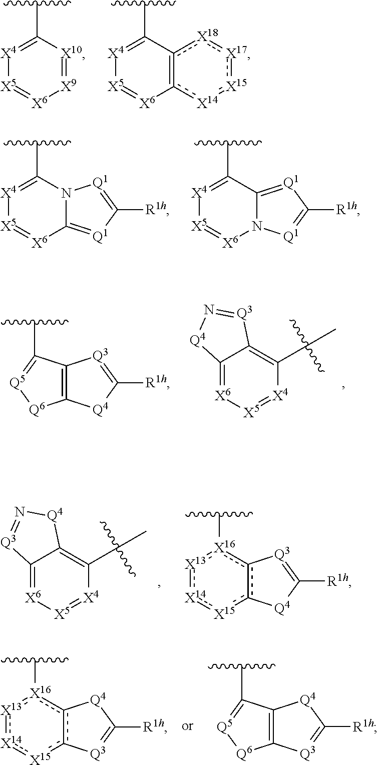

Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, X¹⁸ are independently selected from a C(O), C(R^{1a}), N, C(R^{1a})(R^{1b}), and N(R^{1c});
X¹⁶ are independently selected from C, N, and C(R^{1a});
each R^{1a}, R^{1b}, R^{1d}, and R^{1h} are each independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1h} and one of R^{1a}, R^{1b}, R^{1c}, and R^{id} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.
   Embodiment 113: The compound of any one of embodiments 1 to 108, or a pharmaceutically acceptable salt or solvate thereof, wherein R¹⁹ is selected from:

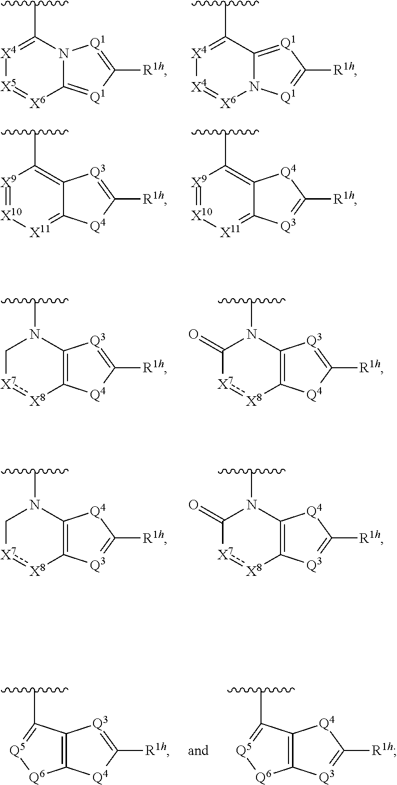

Q¹, Q³, and Q⁵ are independently N or C(R^{1d});
Q⁴ and Q⁶ are independently O, S, C(R^{1a})(R^{1b}), or N(R^{1c});
X⁴, X⁵, X⁶, X⁹, X¹⁰, and X¹¹ are independently selected from C(R^{1a}) and N;
X⁷ and X⁸ are independently selected from C(R^{1a}), C(R^{1a})(R^{1b}), N, and N(R^{1c});
each R^{1a}, R^{1b}, R^{id}, and R^{1h} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ; or two R^{1a} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; or R^{1h} and one of R^{1a}, R^{1b}, R^{1c}, and R^{1d} bonded to adjacent atoms are joined to form a 4-7 membered heterocycloalkyl ring, a phenyl ring, a 5-6 membered heteroaryl ring, or a 4-7 membered cycloalkyl ring, wherein the 4-7 membered heterocycloalkyl ring, phenyl ring, 5-6 membered heteroaryl ring, or 4-7 membered cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.
   Embodiment 114: The compound of any one of embodiments 1 to 108, or a pharmaceutically acceptable salt or solvate thereof, wherein R¹⁹ is selected from

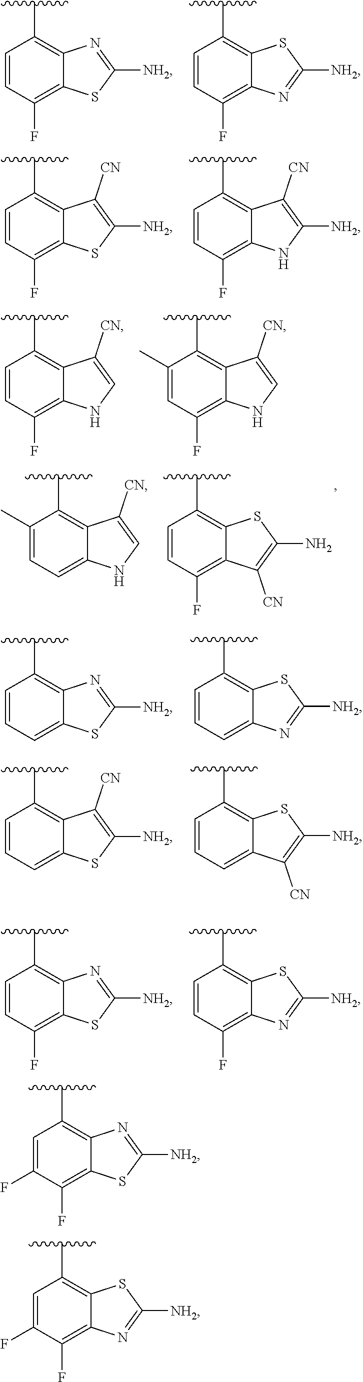

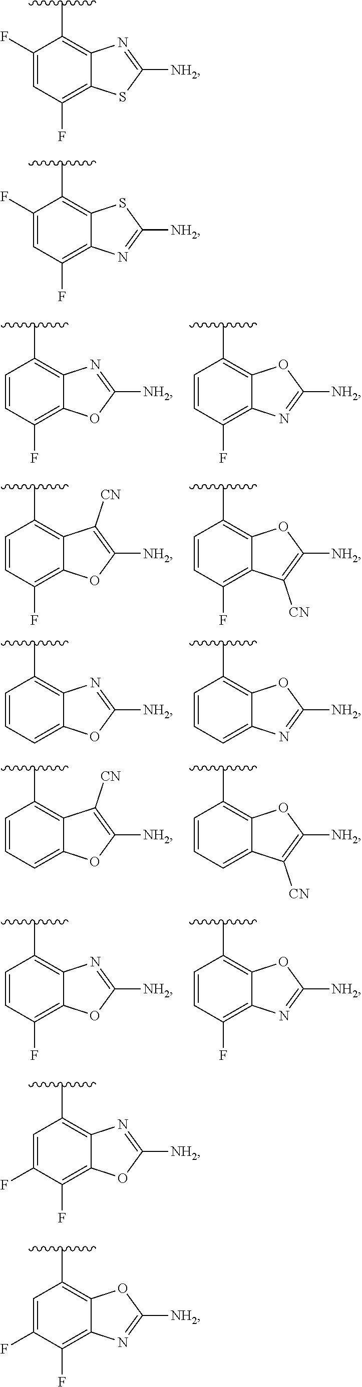

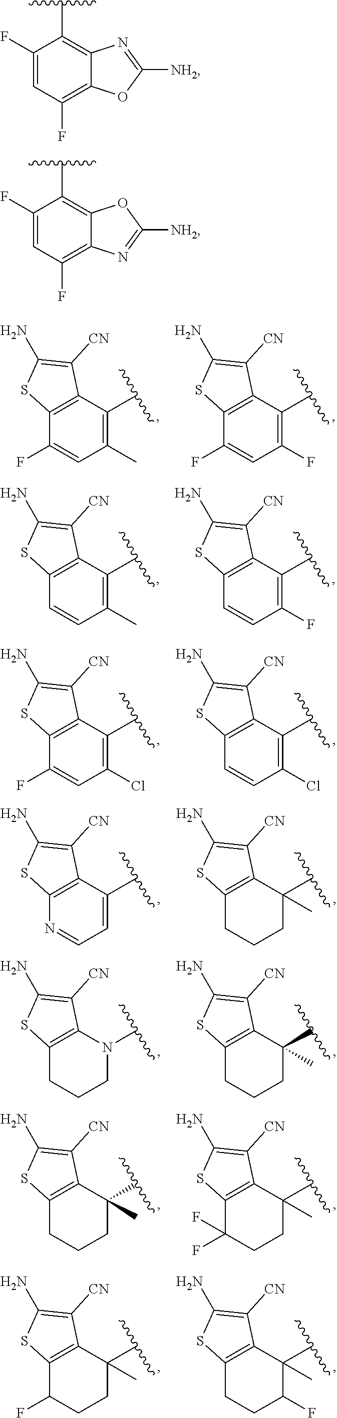

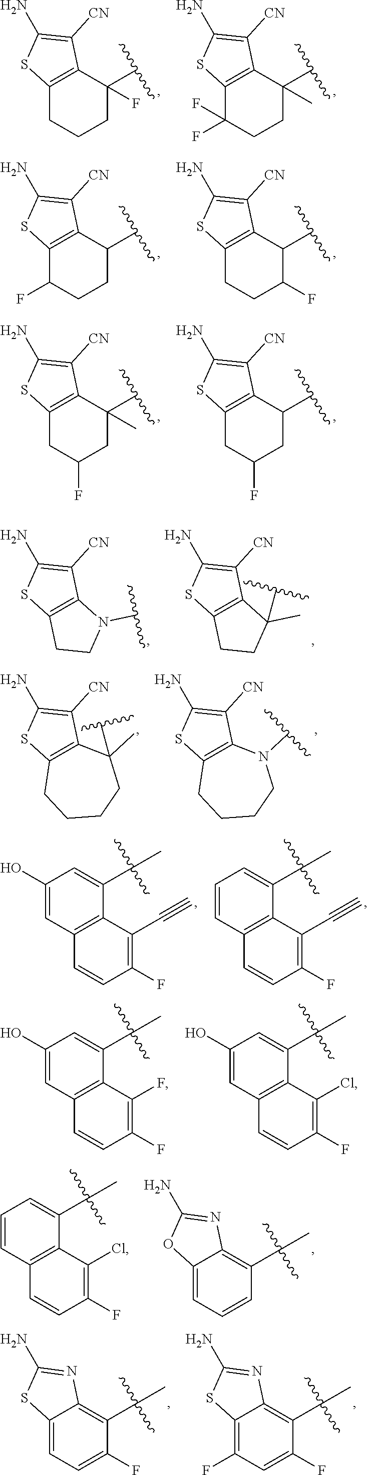

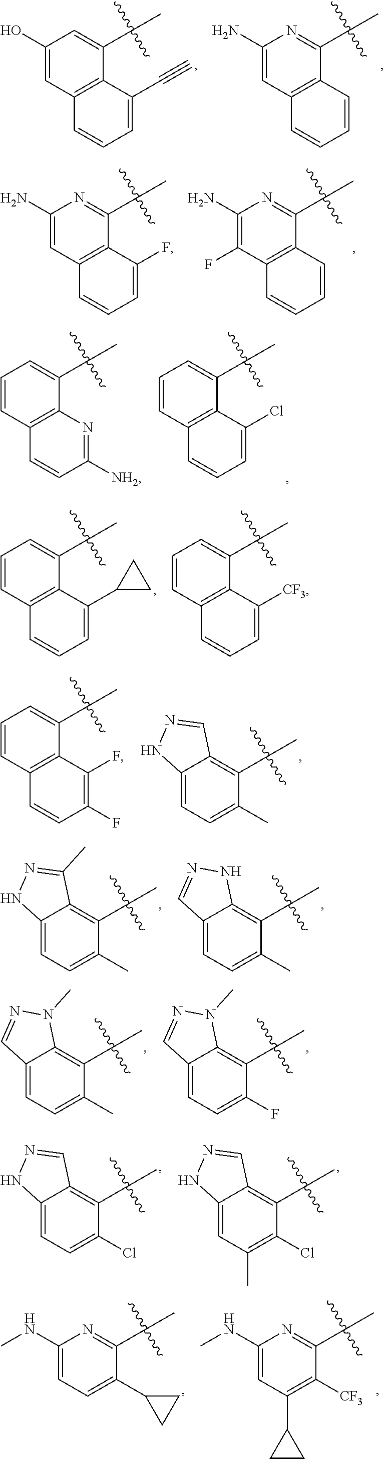

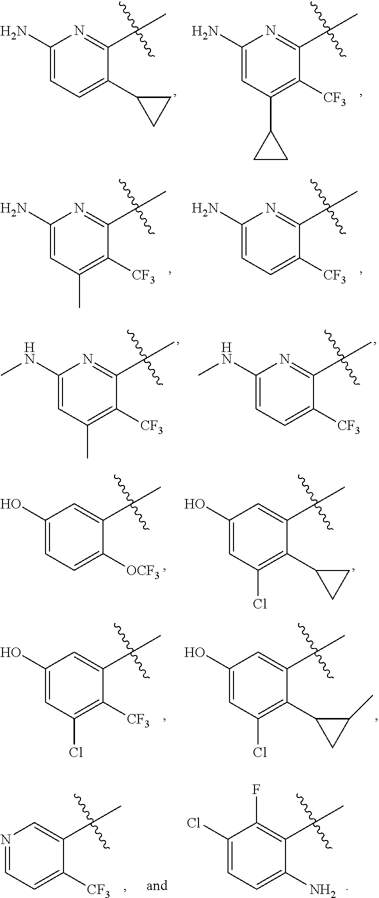

Embodiment 115: The compound of any one of embodiments 1 to 114, or a pharmaceutically acceptable salt or solvate thereof, wherein R² is —OR^{12′}.
Embodiment 116: The compound of any one of embodiments 1 to 114, or a pharmaceutically acceptable salt or solvate thereof, wherein R² is selected from

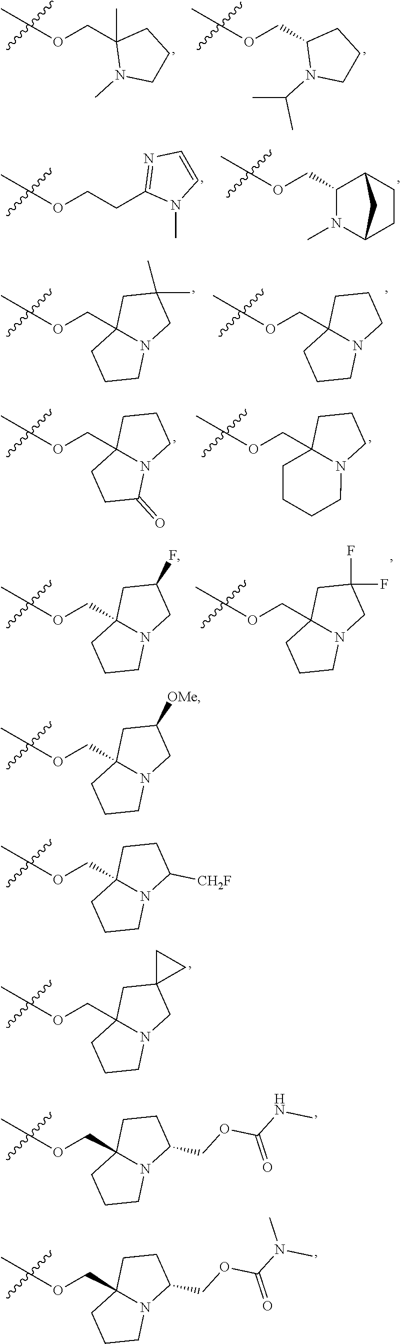

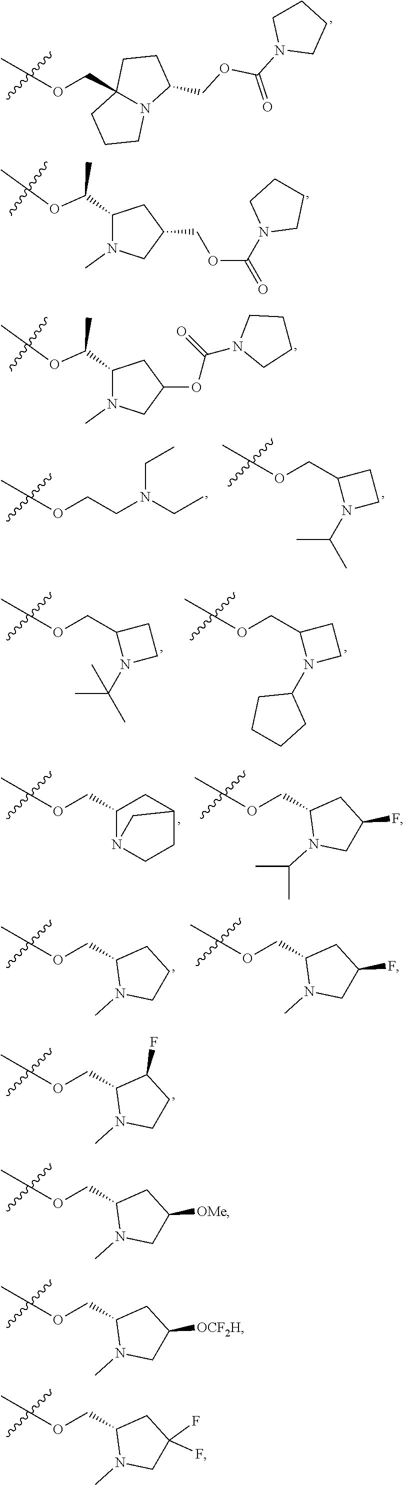

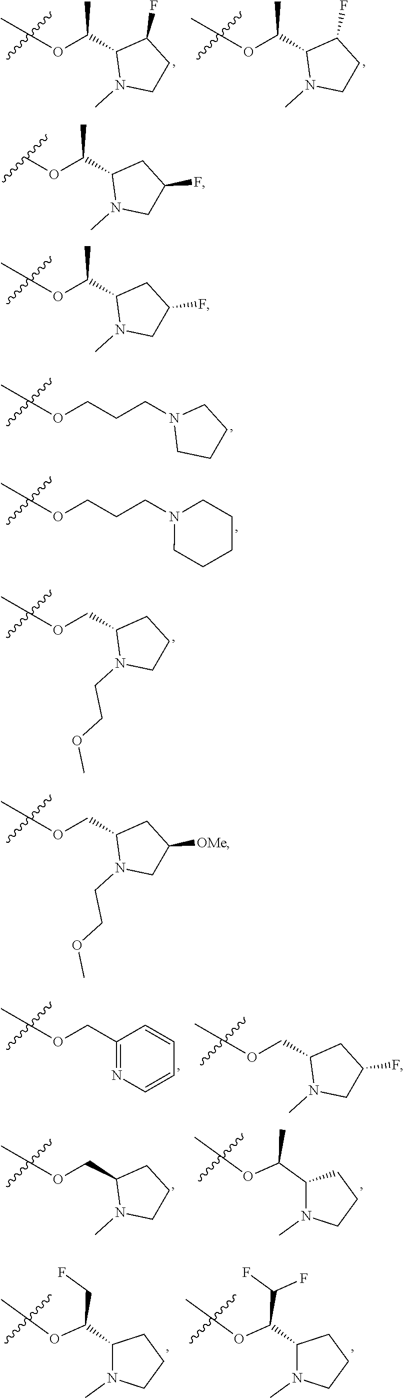

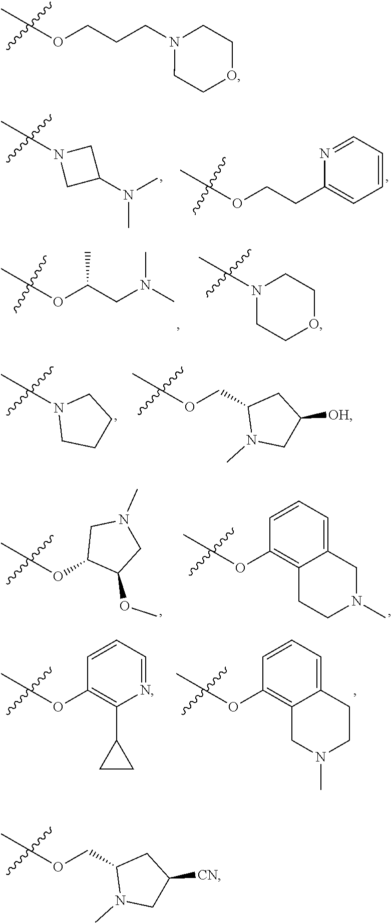

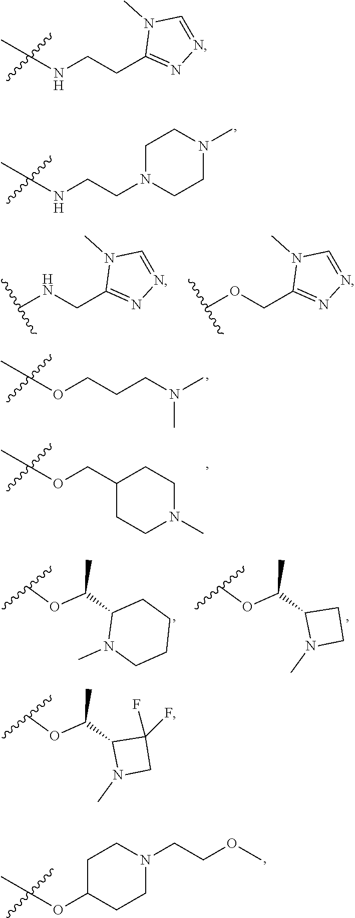

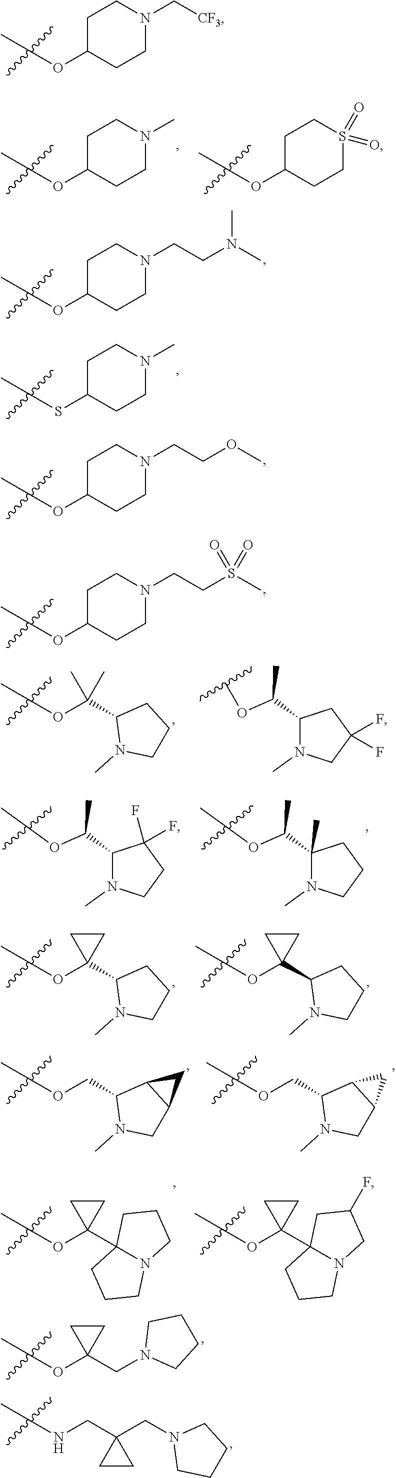

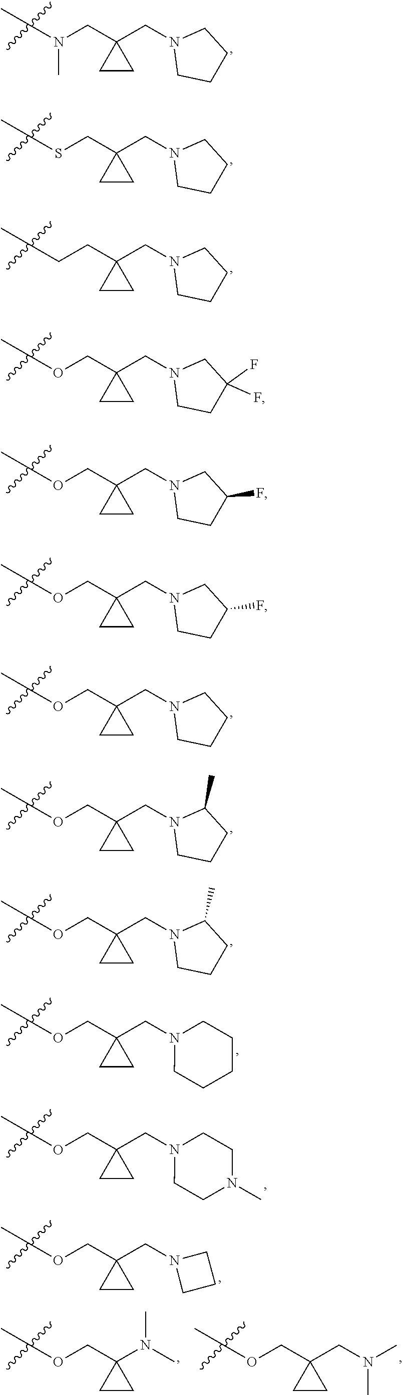

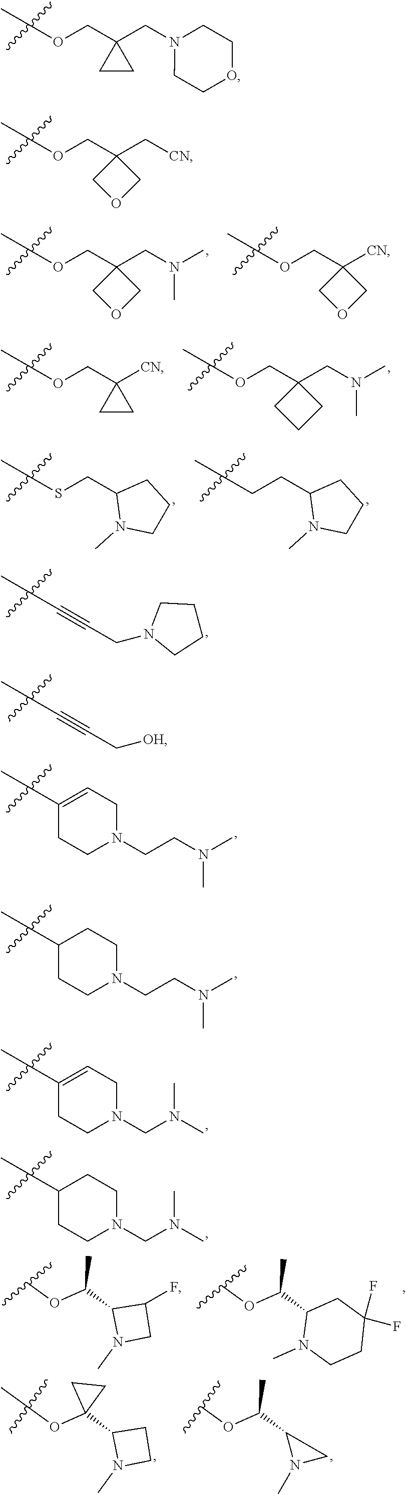

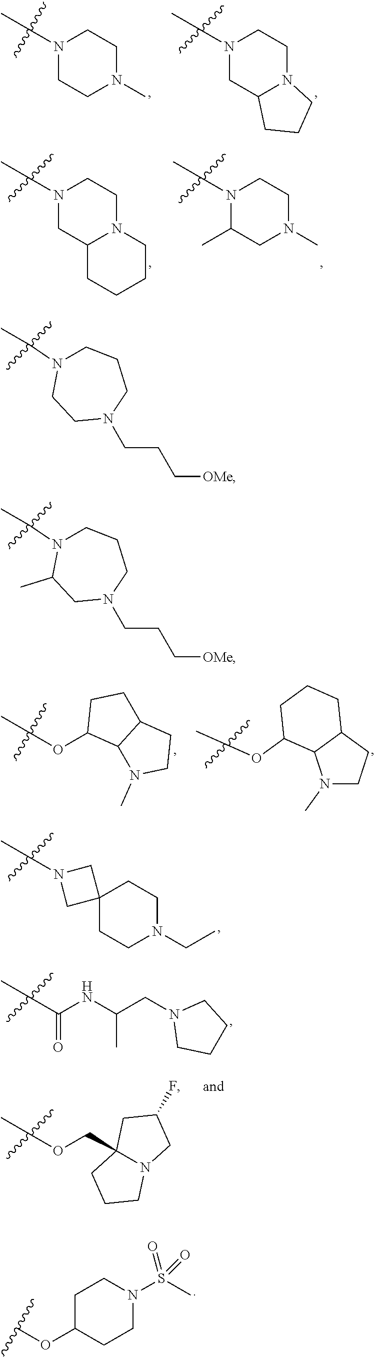

Embodiment 117: A compound having the formula A-L^{AB}-B wherein
A is a monovalent form of a compound of any one of embodiments 1 to 116;
L^{AB} is a covalent linker bonded to A and B; and
B is a monovalent form of a degradation enhancer.
   Embodiment 118: The compound of embodiment 117, wherein the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885.
   Embodiment 119: The compound of embodiment 117, wherein the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1.
   Embodiment 120: The compound of any one of embodiments 117 to 119, wherein L^{AB} is -L^{ABI}-L^{AB2}-L^{AB3}-L^{AB4}-L^{AB5}; L^{AB1}, L^{AB2}, L^{AB3}, L^{AB4}, and L^{AB5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆ alkylene, (—C₁₋₆ alkyl-O)_{z}—, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁ heteroarylene, wherein C₁₋₆alkylene, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁heteroarylene, are optionally substituted with one, two, or three R^{21j}; wherein each C₁₋₆alkyl of (—O—C₁₋₆alkyl)₂- and (—C₁₋₆alkyl-O)₂— is optionally substituted with one, two, or three R^{20j};
z is independently an integer from 0 to 10;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
each R^{20d}, R^{20e}, R^{20f}, and R^{20j} are each independently selected from halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉ heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉ heteroaryl are optionally substituted with one, two, or three groups independently selected from halogen, oxo, —CN, C₁₋₆ alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl.
   Embodiment 121: The compound of any one of embodiments 117 to 119, wherein L^{AB} is —(O—C₂alkyl)_{z}- and z is an integer from 1 to 10.
   Embodiment 122: The compound of any one of embodiments 117 to 119, wherein L^{AB} is —(C₂alkyl-O—)_{z}— and z is an integer from 1 to 10.
   Embodiment 123: The compound of any one of embodiments 117 to 119, wherein L¹ is —(CH₂)_{zz1}L^{AB2}(CH₂O)_{zz2}—, wherein L^{AB2} is a bond, a 5 or 6 membered heterocycloalkylene or heteroarylene, phenylene, —(C₂-C₄)alkynylene, —SO₂— or —NH—; and zz1 and zz2 are independently an integer from 0 to 10.
   Embodiment 124: The compound of any one of embodiments 117 to 119, wherein L^{AB} is —(CH₂)_{zz1}(CH₂O)_{zz2}—, wherein zz1 and zz2 are each independently an integer from 0 to 10.
   Embodiment 125: The compound of any one of embodiments 117 to 119, wherein L^{AB} is a PEG linker.
   Embodiment 126: The compound of any one of embodiments 117 to 125, wherein B is a monovalent form of a compound selected from

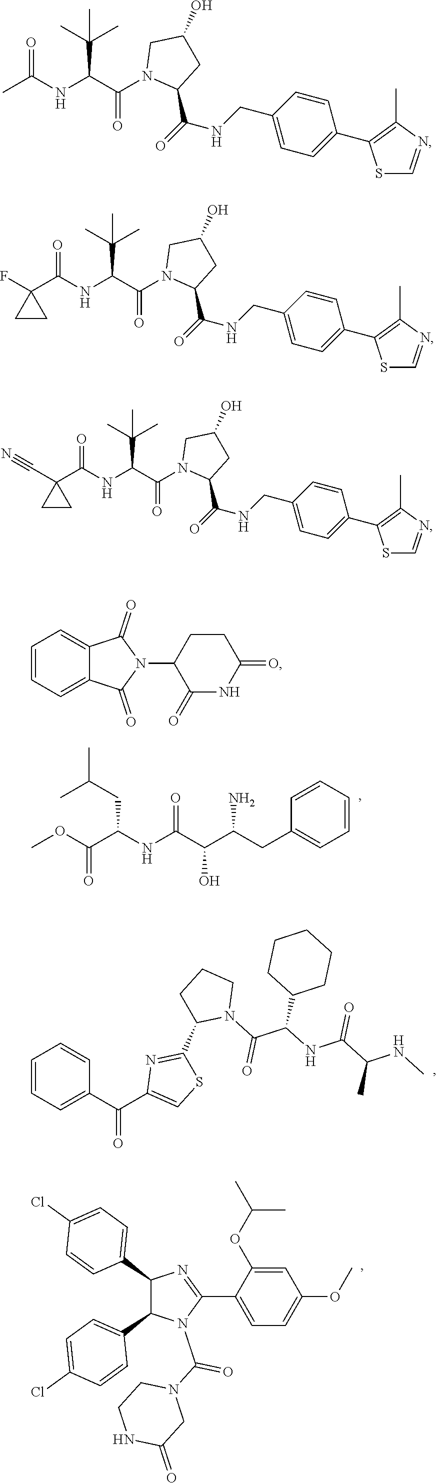

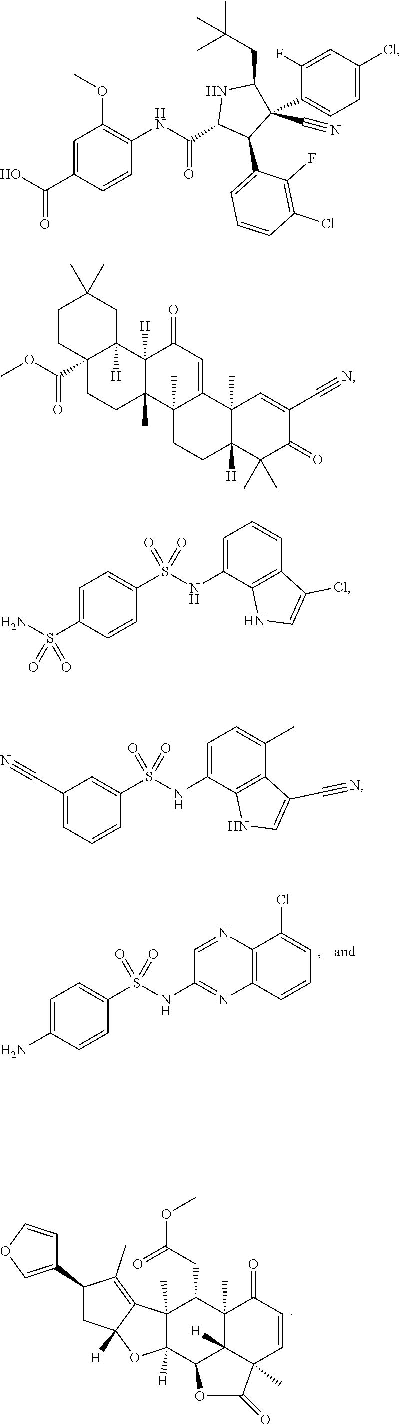

Embodiment 127: A pharmaceutical composition comprising a compound of any one of embodiments 1 to 126, or a pharmaceutically acceptable salt or solvate thereof, and a pharmaceutically acceptable excipient.
Embodiment 128: A method of treating cancer in a subject in need thereof, comprising administering to the subject a therapeutically effective amount of a compound of any one of embodiments 1 to 126, or a pharmaceutically acceptable salt or solvate thereof.
Embodiment 129: A method of treating cancer in a subject comprising a Ras mutant protein, the method comprising: modifying the Ras mutant protein of said subject by administering to said subject a compound, wherein the compound is characterized in that upon contacting the Ras mutant protein, said Ras mutant protein is modified covalently at a residue corresponding to reside 12 of SEQ ID No: 1, such that said modified Ras mutant protein exhibits reduced Ras signaling output.
Embodiment 130: The method of any one of embodiments 128 to 129, wherein the cancer is a solid tumor or a hematological cancer.
Embodiment 131: The method of any one of embodiments 128 to 129, wherein the Ras mutant protein is K-Ras G12S.
Embodiment 132: The method of any one of embodiments 128 to 129, wherein the compound is a compound of any one of embodiments 1 to 126.
Embodiment 133: A method of modulating signaling output of a Ras protein, comprising contacting a Ras protein with an effective amount of a compound of any one of embodiments 1 to 126, or a pharmaceutically acceptable salt or solvate thereof, thereby modulating the signaling output of the Ras protein.
Embodiment 134: A method of inhibiting cell growth, comprising administering an effective amount of a compound of any one of embodiments 1 to 126, or a pharmaceutically acceptable salt or solvate thereof, to a cell expressing a Ras protein, thereby inhibiting growth of said cells.
Embodiment 135: The method of embodiment any one of embodiments 128 to 134, comprising administering an additional agent.
Embodiment 136: The method of embodiment 135, wherein the additional agent comprises (1) an inhibitor of MEK; (2) an inhibitor of epidermal growth factor receptor (EGFR) and/or of mutants thereof; (3) an immunotherapeutic agent; (4) a taxane; (5) an anti-metabolite; (6) an inhibitor of FGFR1 and/or FGFR2 and/or FGFR3 and/or of mutants thereof; (7) a mitotic kinase inhibitor; (8) an anti-angiogenic drug; (9) a topoisomerase inhibitor; (10) a platinum-containing compound; (12) an inhibitor of c-MET and/or of mutants thereof; (13) an inhibitor of BCR-ABL and/or of mutants thereof; (14) an inhibitor of ErbB2 (Her2) and/or of mutants thereof; (15) an inhibitor of AXL and/or of mutants thereof; (16) an inhibitor of NTRK1 and/or of mutants thereof; (17) an inhibitor of RET and/or of mutants thereof; (18) an inhibitor of A-Raf and/or B-Raf and/or C-Raf and/or of mutants thereof; (19) an inhibitor of ERK and/or of mutants thereof; (20) an MDM2 inhibitor; (21) an inhibitor of mTOR20i; (23) an inhibitor of IGF1/2 and/or of IGF1-R²⁰ⁱ; (24) an inhibitor of CDK9; (25) an inhibitor of farnesyl transferase; (26) an inhibitor of SHIP pathway; (27) an inhibitor of SRC; (28) an inhibitor of JAK; (29) a PARP inhibitor, (31) a ROS1 inhibitor; (32) an inhibitor of SHP pathway, or (33) an inhibitor of Src, FLT3, HDAC, VEGFR, PDGFR, LCK, Bcr-Abl or AKT; (34) an inhibitor of KrasG12C mutant; (35) a SHC inhibitor (e.g., PP2, AID371185); (36) a GAB inhibitor; (38) a PI-3 kinase inhibitor; (39) a MARPK inhibitor; (40) CDK4/6 inhibitor; (41) MAPK inhibitor; (42) SHP2 inhibitor; (43) checkpoint immune blockade agents; (44) or SOS1 inhibitor; or (45) a SOS 2 inhibitor.
Embodiment 137: The method of embodiment 135, wherein the additional agent comprises an inhibitor of SHP2 selected RMC-4630, ERAS-601,

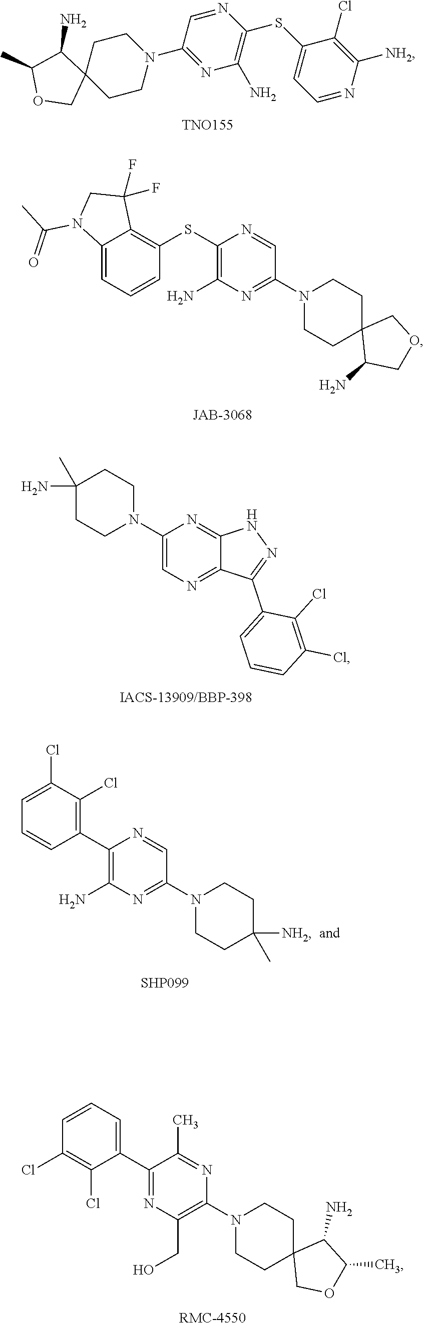

Embodiment 138: The method of embodiment 135, wherein the additional agent comprises an inhibitor of SOS selected from

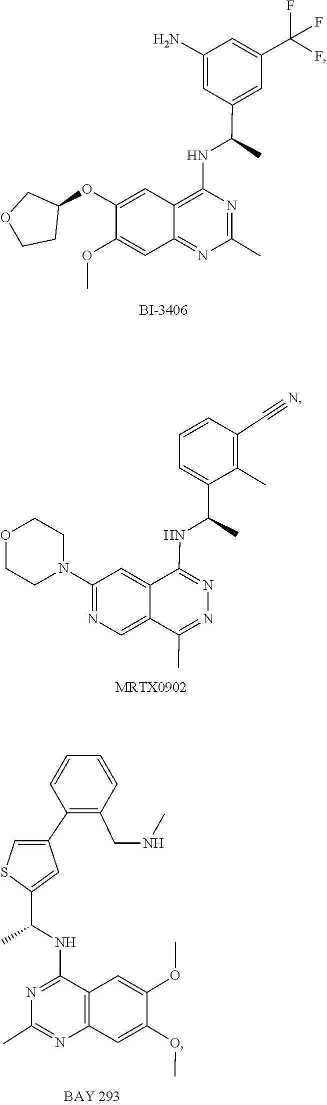

### RMC-5845, and BI-1701963.

Embodiment 139: The method of embodiment 135, wherein the additional agent comprises an inhibitor of EGFR selected from afatinib, erlotinib, gefitinib, lapatinib, cetuximab panitumumab, osimertinib, olnutinib, and EGF-816.
Embodiment 140: The method of embodiment 135, wherein the additional agent comprises an inhibitor of MEK selected from trametinib, cobimetinib, binimetinib, selumetinib, refametinib, and AZD6244.
Embodiment 141: The method of embodiment 135, wherein the additional agent comprises an inhibitor of ERK selected from ulixertinib, MK-8353, LTT462, AZD0364, SCH772984, BIX02189, LY3214996, and ravoxertinib.
Embodiment 142: The method of embodiment 135, wherein the additional agent comprises an inhibitor of CDK4/6 selected from palbociclib, ribociclib, and abemaciclib.
Embodiment 143: The method of embodiment 135, wherein the additional agent comprises an inhibitor of BRAF selected from Sorafenib, Vemurafenib, Dabrafenib, Encorafenib, regorafenib, and GDC-879.

### EXAMPLES

The following examples are provided for illustrative purposes only and not to limit the scope of the claims provided herein.

As used herein, the following abbreviations, unless otherwise indicated, shall be understood to have the following meanings:
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
i-Bu iso-butyl
t-Bu tert-butyl
DCM dichloromethane (CH₂Cl₂)
DIBAL-H diisobutylaluminum hydride
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMSO dimethylsulfoxide
Dppf or dppf 1,1′-bis(diphenylphosphino)ferrocene
EDC or EDCI N-(3-dimethylaminopropyl)-N′-ethylcarbodiimide hydrochloride
eq equivalent(s)
Et ethyl
Et₂O diethyl ether
EtOH ethanol
EtOAc ethyl acetate
HPLC high performance liquid chromatography
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
Me methyl
MeOH methanol
MS mass spectroscopy
Ms mesyl
NMR nuclear magnetic resonance
Ph phenyl
iPr/i-Pr iso-propyl
RP-HPLC reverse-phase high-pressure liquid chromatography
RT room temperature
TBS tert-butyldimethylsilyl
TEA triethylamine
TFA trifluoroacetic acid
THF tetrahydrofuran
TLC thin layer chromatography
TMS trimethylsilyl
TsOH/p-TsOH p-toluenesulfonic acid.

Example 1: General Syntheses: Compounds described herein may be synthesized using the synthetic schemes described below, or variations thereof, and/or well known and understood synthetic schemes.

### Example A: Synthesis of 2-Amino-4-(6-chloro-4-(1-(2,2-difluorocyclopropane-1-carbonyl)-1,6-diazaspiro[3.5]nonan-6-yl)-8-fluoro-2-((S)-1-((S)-1-methylpyrrolidin-2-yl)ethoxy)quinazolin-7-yl)-7-fluorobenzo[b]thiophene-3-carbonitrile (223)

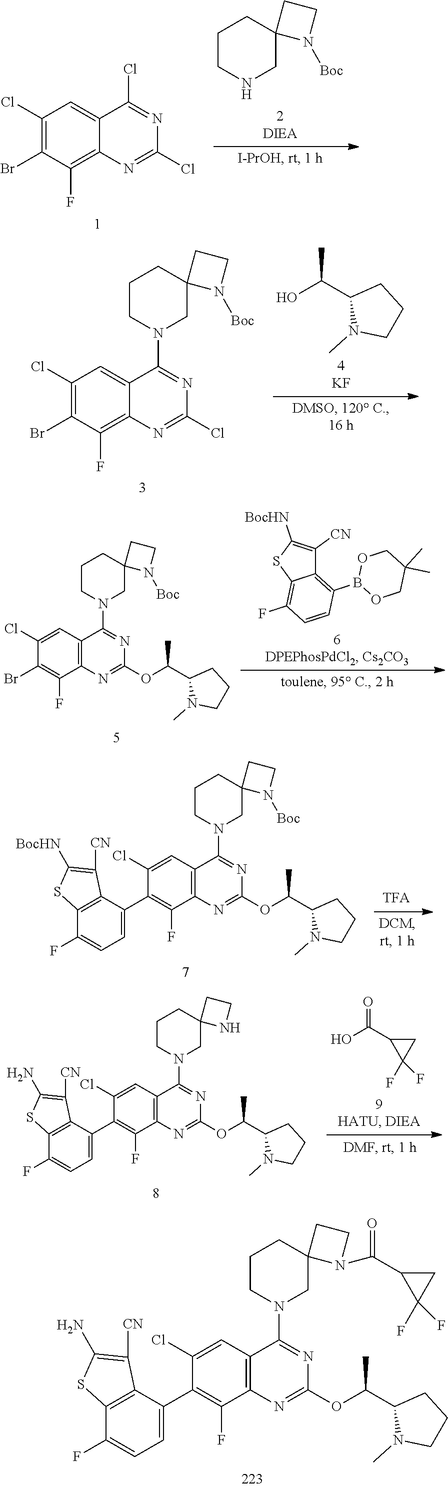

To a solution of compound 1 (1 g, 3.03 mmol) and compound 2 (685 mg, 3.03 mmol) in i-PrOH (15 mL) was added N,N-Diisopropylethylamine (1.17 g, 9.09 mmol). The reaction mixture was stirred at 25° C. for 1 h. LCMS showed the reaction was completed. The mixture was poured into ice water (200 mL). The mixture was filtered and the solid was diluted with PE (150 mL). The organic solution was concentrated to dryness under reduced pressure to give compound 3 (1.3 g, Yield: 83%) as a white solid. MS m/z (ESI): 519.0 [M+H]⁺.

To a solution of compound 3 (1.3 g, 2.5 mmol) in DMSO (10 mL) were added compound 4 (1.61 g, 12.5 mmol) and KF (725 mg, 12.5 mmol). The reaction mixture was then stirred at 120° C. for 16 h. LCMS showed the reaction was completed. The mixture was poured into ice water (100 mL), and the solution was extracted with ethyl acetate (100 mL×3). The combined organic layers were washed with brine (100 mL), dried over anhydrous sodium sulfate, filtered and the filtrate was concentrated to dryness under reduced pressure. The crude was purified by SGC (PE/EA=1/1) to give compound 5 (500 mg, Yield: 33%) as a yellow solid. MS m/z (ESI): 612.2 [M+H]⁺.

To a solution of compound 5 (500 mg, 0.82 mmol), compound 6 (492 mg, 1.22 mmol), and Cs₂CO₃ (802 mg, 2.46 mmol) in toluene (20 mL) was added DPEPhosPdCl₂ (117 mg, 0.16 mmol). The mixture was stirred at 95° C. under nitrogen for 2 hours. LCMS showed the reaction was completed. The mixture was poured into water (200 mL), and the solution was extracted with ethyl acetate (200 mL×3). The combined organic layers were washed with brine (100 mL), dried over anhydrous sodium sulfate, filtered and the filtrate was concentrated to dryness under reduced pressure. The crude was purified by SGC (PE/EA=1/1) to give compound 7 (290 mg, Yield: 43%) as a white solid. MS m/z (ESI): 824.2 [M+H]⁺.

To a solution of compound 7 (180 mg, 0.22 mmol) in DCM (2 mL) was added TFA (1 mL). The reaction mixture was stirred at rt for 1 h. LCMS showed the reaction was completed. The reaction mixture was concentrated under the reduced pressure. The residue was diluted with dichloromethane (100 mL). The solution was basified with NaHCO₃ solution. The organic layer was washed with brine, dried over anhydrous sodium sulfate, filtered, and concentrated to dryness under reduced pressure to give compound 8 (130 mg, Yield: 96%) as a white solid. MS m/z (ESI): 624.3 [M+H]⁺.

To a solution of compound 9 (39 mg, 0.32 mmol) and DIEA (83 mg, 0.64 mmol) in DMF (5 mL) was added HATU (183 mg, 0.48 mmol) at 0° C. The reaction mixture was then stirred at rt for 30 min. To a solution of compound 8 (90 mg, 0.144 mmol) in DMF (1 mL) was added above reaction mixture (1.7 mL). The mixture was stirred at 25° C. for 1 h. LCMS showed the reaction was completed. The mixture was poured into water (50 mL), and the solution was extracted with ethyl acetate (50 mL×3). The combined organic layers were washed with brine (50 mL), dried over anhydrous sodium sulfate, filtered and the filtrate was concentrated to dryness under reduced pressure. The crude was purified by prep-HPLC to give 223 (22.86 mg, Yield: 22%). MS: 728.4 [M+H]⁺.

1H NMR (400 MHz, DMSO-d₆) δ 8.18 (s, 1H), 8.12 (s, 2H), 7.89-7.79 (m, 1H), 7.33-7.23 (m, 1H), 7.20-7.11 (m, 1H), 5.32-5.22 (m, 1H), 4.49-4.32 (m, 1H), 4.26-3.96 (m, 3H), 3.92-3.71 (m, 2H), 3.25-3.14 (m, 1H), 3.02 (d, J=5.6 Hz, 1H), 2.79 (d, J=6.4 Hz, 1H), 2.70-2.56 (m, 1H), 2.44 (d, J=6.8 Hz, 3H), 2.40-2.16 (m, 2H), 2.11-1.96 (m, 2H), 1.91-1.61 (m, 8H), 1.28 (t, J=6.2 Hz, 3H).

### Example B: Synthesis of 2-amino-4-(6-chloro-8-fluoro-2-(((2R,7aS)-2-fluorotetrahydro-1H-pyrrolizin-7a(5H)-yl)methoxy)-4-(6-(3-methyl-1H-1,2,4-triazole-1-carbonyl)-9-oxa-2,6-diazaspiro[4.5]decan-2-yl)quinazolin-7-yl)-7-fluorobenzo[b]thiophene-3-carbonitrile (214)

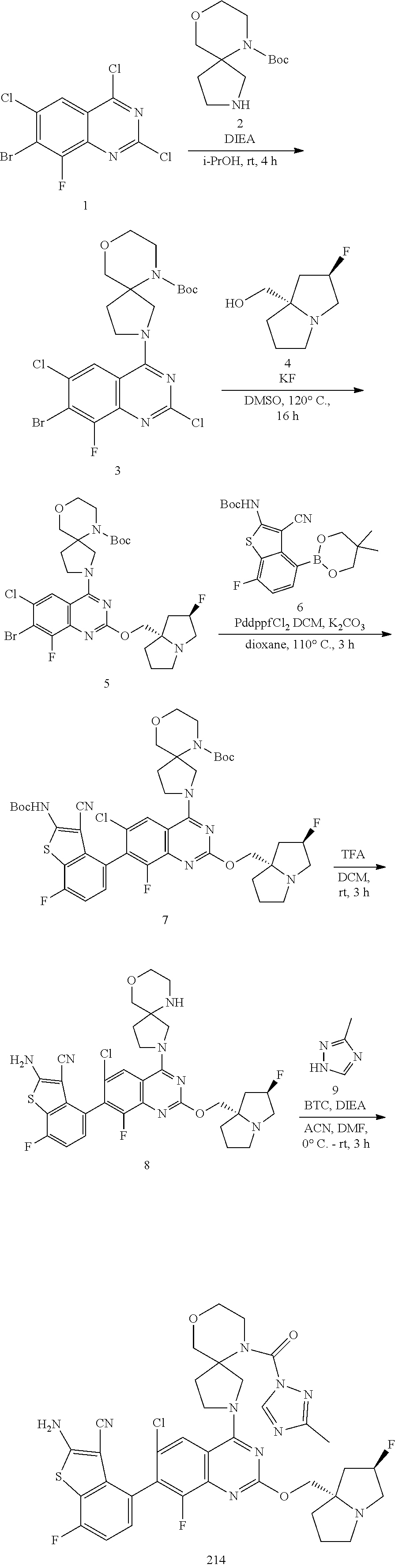

To a solution of compound 1 (680 mg, 2.06 mmol) and compound 2 (499 mg, 2.06 mmol) in i-PrOH (8 mL) was added DIEA (799 mg, 6.18 mmol). The reaction mixture was stirred at 25° C. for 4 h. The mixture was poured into ice water (100 mL). The mixture was filtered and the solid was diluted with MeOH (100 mL). The organic solution was concentrated to dryness under reduced pressure to give compound 3 (1.0 g, Yield: 90.9%) as a yellow solid. MS m/z (ESI): 535.1 [M+H]⁺.

To a solution of compound 3 (950 mg, 1.77 mmol) and compound 4 (1.41 g, 8.86 mmol) in DMSO (10 mL) was added KF (514 mg, 8.86 mmol). The reaction mixture was stirred at 120° C. for 16 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 5 (400 mg, Yield: 34.4%) as a yellow solid. MS m/z (ESI): 658.2 [M+H]⁺.

To a solution of compound 5 (400 mg, 0.61 mmol), compound 6 (410 mg, 1.22 mmol), and K₂CO₃ (506 mg, 3.66 mmol) in 1,4-dioxane (15 mL) was added PddppfCl₂ DCM (49 mg, 0.06 mmol). The mixture was stirred at 110° C. under nitrogen for 3 hours. The mixture was poured into water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 7 (430 mg, Yield: 81.13%) as a white solid. MS m/z (ESI): 870.3 [M+H]⁺.

To a solution of compound 7 (170 mg, 0.20 mmol) in DCM (10 mL) was added TFA (5 mL). The reaction mixture was stirred at rt for 3 h. The solvent was removed under the reduced pressure. The resulting mixture was diluted with a.q. NaHCO₃ (20 mL) and EA (50 mL). The organic layer was washed with brine, dried over sodium sulfate, filtered, and concentrated to dryness under reduced pressure to give compound 8 (130 mg, Yield: 99.3%) as a yellow solid. MS m/z (ESI): 670.2 [M+H]⁺.

To a solution of compound 9 (62 mg, 0.75 mmol) and DIEA (0.5 mL, 3.025 mmol) in MeCN (4.5 mL) was added triphosgene (200 mg, 0.675 mmol) at 0° C. under N₂ atmosphere. The reaction mixture was then stirred at 20° C. for 2 h. To a solution of compound 8 (40 mg, 0.06 mmol) in DMF (1 mL) was added above reaction mixture (0.4 mL) at 20° C. under N₂ atmosphere. The crude was purified by prep-HPLC (FA) to give 214 (2.23 mg, Yield: 4.8%) as a white solid. MS m/z (ESI): 779.3 [M+H]⁺.

¹H NMR (400 MHz, DMSO-d₆) δ 8.95 (d, J=4.0 Hz, 1H), 8.15 (s, 1H), 8.11 (s, 2H), 7.29-7.20 (m, 1H), 7.15 (t, J=9.2 Hz, 11H), 5.27 (d, J=54.4 Hz, 11H), 4.48-4.31 (m, 11H), 4.29-4.20 (m, 11H), 4.21-4.05 (m, 3H), 4.05-3.96 (m, 11H), 3.91-3.65 (m, 7H), 3.14-3.02 (m, 3H), 3.03-2.98 (m, 1H), 2.89-2.76 (m, 2H), 2.56 (s, 1H), 2.33 (d, J=2.4 Hz, 3H), 2.16-2.10 (m, 1H), 2.07-2.03 (m, 1H), 2.03-1.96 (m, 1H), 1.90-1.70 (m, 3H).

### Example C: Synthesis of 2-amino-4-(6-chloro-4-(1-((1R,2R)-2-cyanocyclopropane-1-carbonyl)-1,6-diazaspiro[3.4]octan-6-yl)-8-fluoro-2-((S)-1-((S)-1-methylpyrrolidin-2-yl)ethoxy)quinazolin-7-yl)-7-fluorobenzo[b]thiophene-3-carbonitrile (206)

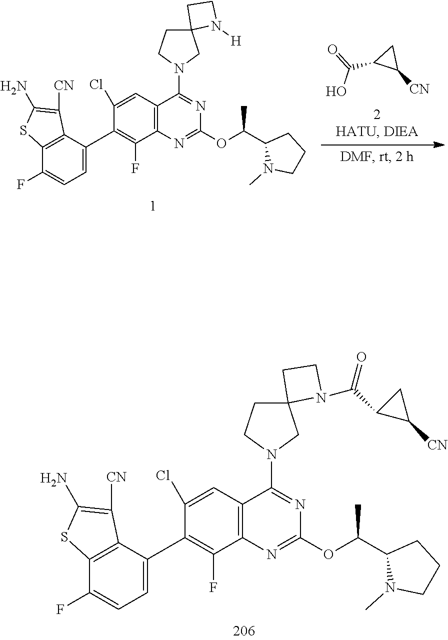 [SMILES: C[C@H](Oc1nc(N2CCC3(CCN3)C2)c2cc(Cl)c(-c3ccc(F)c4sc(N)c(C#N)c34)c(F)c2n1)[C@@H]1CCCN1C ; N#C[C@@H]1C[C@H]1C(=O)O ; C[C@H](Oc1nc(N2CCC3(CCN3C(=O)[C@@H]3C[C@H]3C#N)C2)c2cc(Cl)c(-c3ccc(F)c4sc(N)c(C#N)c34)c(F)c2n1)[C@@H]1CCCN1C]

To a solution of compound 1 (40 mg, 0.066 mmol), HATU (14 mg, 0.099 mmol) and DIEA (17 mg, 0.132 mmol) in DMF (1 mL), was added compound 2 (7 mg, 0.066 mmol). The reaction mixture was then stirred at rt for 2 h. LCMS showed the reaction was completed. The crude was purified by prep-HPLC to give 206 (5.23 mg, Yield: 11.4%). MS m/z (ESI): 703.21 [M+H]⁺.

¹H NMR (400 MHz, DMSO-d₆) δ 8.12 (s, 2H), 8.08 (s, 1H), 7.26-7.20 (m, 1H), 7.18-7.12 (m, 1H), 5.35-5.20 (m, 1H), 4.50-3.70 (m, 6H), 3.00-2.95 (m, 11H), 2.70-2.55 (m, 2H), 2.45-2.25 (m, 5H), 2.24-2.15 (m, 2H), 2.12-1.95 (m, 11H), 1.79-1.62 (m, 4H), 1.39 (d, J=10.0 Hz, 1H), 1.31-1.17 (m, 511).

### Example D; 2-amino-4-(6-chloro-8-fluoro-4-(1-(2-methoxyacetyl)-1,6-diazaspiro[3.4]octan-6-yl)-2-((S)-1-((S)-1-methylpyrrolidin-2-yl)ethoxy)quinazolin-7-yl)-7-fluorobenzo[b]thiophene-3-carbonitrile 254

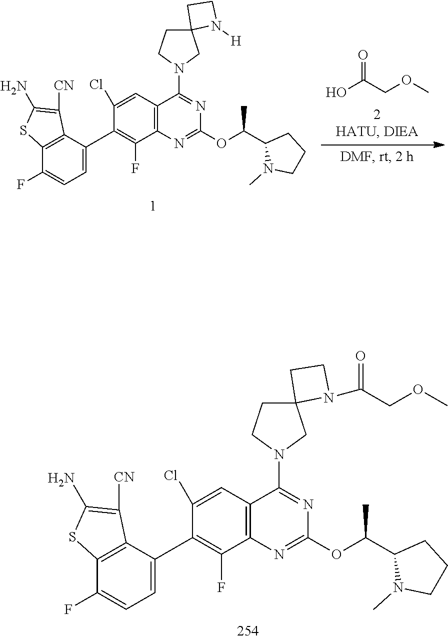

To a solution of compound 1 (40 mg, 0.066 mmol), HATU (14 mg, 0.099 mmol) and N,N-Diisopropylethylamine (17 mg, 0.132 mmol) in DMF (1 mL), was added compound 2 (6 mg, 0.066 mmol). The reaction mixture was then stirred at rt for 2 h. LCMS showed the reaction was completed. The crude was purified by prep-HPLC to give 254 (2.02 mg, Yield: 4.5%). MS m/z (ESI): 682.19 [M+H]⁺.

¹H NMR (400 MHz, DMSO-d₆) δ 8.11 (s, 2H), 8.09 (s, 1H), 7.26-7.22 (m, 1H), 7.17-7.12 (m, 1H), 5.30-5.25 (m, 1H), 4.45 (s, 1H), 4.25-3.80 (m, 7H), 3.27 (s, 3H), 3.00-2.90 (m, 1H), 2.70-2.55 (m, 2H), 2.40-2.30 (m, 4H), 2.23-2.13 (m, 2H), 1.80-1.50 (m, 4H), 1.24 (d, J=6.4 Hz, 3H), 1.17 (d, J=6.8 Hz, 1H).

### Example E: Synthesis of (6-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,6-diazaspiro[3.4]octan-1-yl)(1H-1,2,4-triazol-1-yl)methanone (Compound 270))

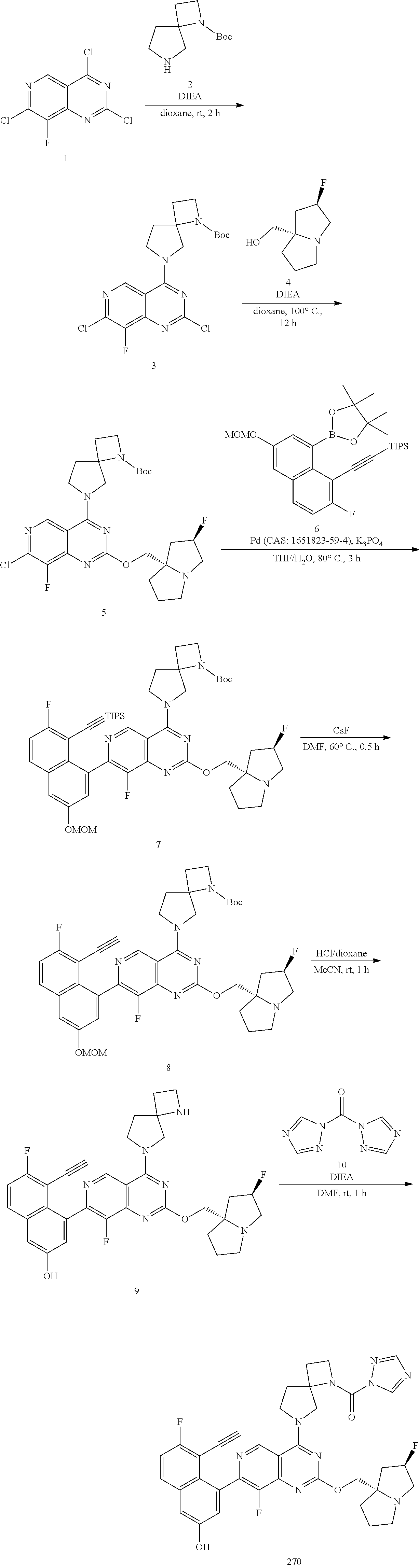

To a solution of compound 2 (700 mg, 3.30 mmol) and N,N-Diisopropylethylamine (853 mg, 6.60 mmol) in dry dioxane (4 mL) was added compound 1 (828 mg, 3.30 mmol). The reaction was stirred at room temperature for 2 hr. The mixture was poured into ice water (100 mL). The mixture was filtered and the solid was collected to obtain compound 3 as a yellow solid (1.2 g, Yield: 85%). MS m/z (ESI): 458.2 [M+H]⁺.

To a solution of compound 3 (520 mg, 1.22 mmol) and N,N-Diisopropylethylamine (473 mg, 3.66 mmol) in dioxane (10 ml) was added compound 4 (582 mg, 3.66 mmol). The reaction mixture was stirred at 100° C. for 12 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=20/1) to give compound 5 (350 mg, Yield: 52%) as a white solid. MS m/z (ESI): 552.4 [M+H]⁺.

To a solution of compound 5 (270 mg, 0.49 mmol), compound 6 (302 mg, 0.59 mmol) and K₃PO₄ 312 mg, 1.47 mmol) in dry THF/H₂O (8 mL/2 mL) was added calcium A Pd G3 (364 mg, 0.5 mmol). The mixture was stirred at 80° C. for 3 hr under N₂. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (EA:MeOH=20:1) to give compound 7 (250 mg, Yield: 57%) as a white solid. MS m/z (ESI): 901.4 [M+H]⁺.

To a solution of compound 7 (100 mg, 0.11 mmol) in dry DMF (5 mL) was added CsF (167 mg, 152 mmol). The mixture was stirred at 60° C. for 0.5 hr. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure to give compound 8 (70 mg, Yield: 85%) as a white solid. MS m/z (ESI): 745.5 [M+H]⁺.

To a solution of compound 8 (100 mg, 0.13 mmol) in CH₃CN (3 mL) was added HCl/dioxane (3 mL). The solution was stirred at 20° C. for 1 h. Then the solution was added dropwise to n-hexane (50 mL), the upper clear liquid was removed and the residue was dissolved in a solution of DCM (100 mL) and MeOH (5 mL). The organic layer was washed by aqueous a.q. NaHCO₃, and brine. The organic layer was separated, dried over Na₂SO₄, concentrated to give compound 9 (70 mg, Yield: 87%) as a yellow solid. MS m/z (ESI): 601.3 [M+H]⁺.

To a solution of compound 9 (50 mg, 0.08 mmol) and DIEA (39 mg, 0.24 mmol) in DMF (0.5 mL), was added compound 10 (31 mg, 0.24 mmol). The reaction mixture was then stirred at rt for 1 h. LCMS showed the reaction was completed. The crude was purified by prep-HPLC to give 270 (5.38 mg, Yield: 9.3%). MS m/z (ESI): 696.3 [M+H]⁺.

1H NMR (400 MHz, DMSO-d₆) δ 10.16 (s, 1H), 9.24 (d, J=6.8 Hz, 1H), 9.15 (d, J=5.8 Hz, 1H), 8.27 (d, J=6.2 Hz, 1H), 8.17 (s, 1H), 7.98 (m, 1H), 7.46 (t, J=9.0 Hz, 1H), 7.39 (d, J=2.4 Hz, 1H), 7.18 (s, 1H), 5.28 (d, J=54.4 Hz, 1H), 4.67-3.95 (m, 8H), 3.14-2.87 (m, 4H), 2.48-1.70 (m, 10H).

### Example F: Synthesis of 2-amino-4-(6-chloro-4-(3,3-difluoro-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (232)

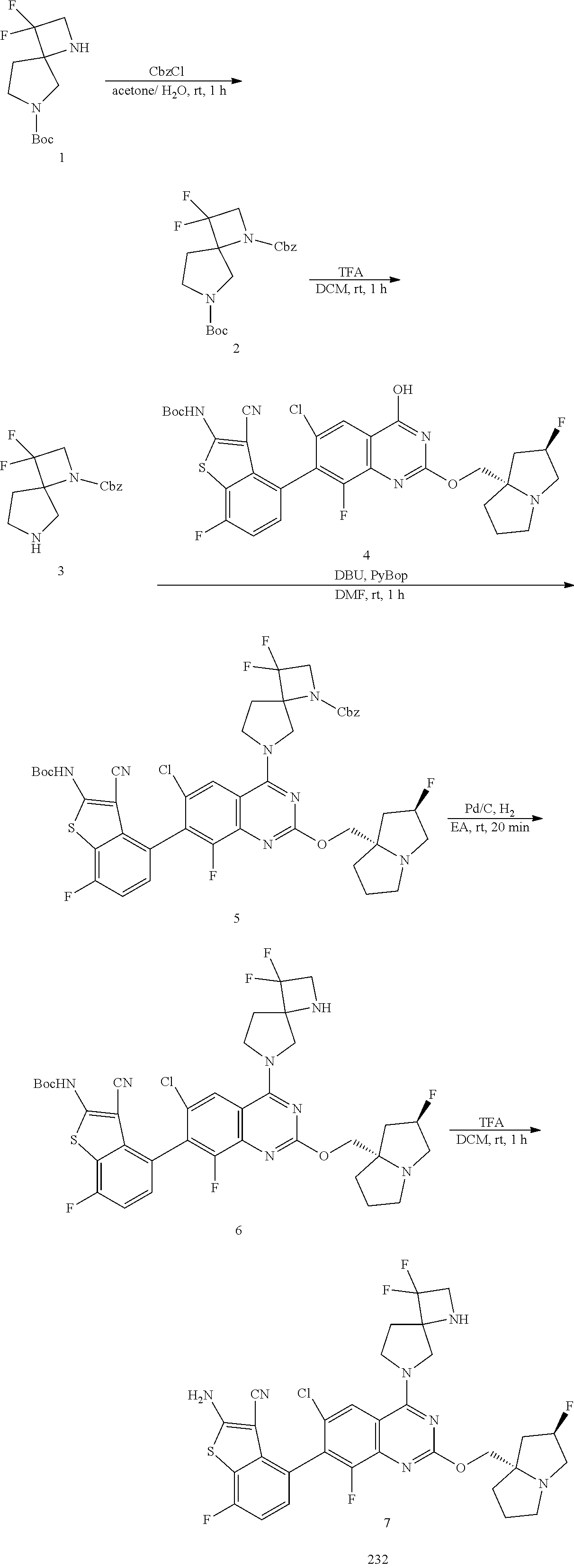

To a solution of compound 1 (100 mg, 0.40 mmol) and NaHCO₃(68 mg, 0.80 mmol) in acetone (2 mL) and H₂O (1 mL) was added CbzCl (137 mg, 0.80 mmol). The mixture was stirred at 25° C. for 1 h. The solution was extracted with ethyl acetate (50 mL×3). The organic layer was washed with brine (50 mL), dried over anhydrous sodium sulfate, filtered and the filtrate was concentrated to dryness under reduced pressure. The residue was purified by silica gel column (PE/EA=6/1) to give compound 2 (80 mg, Yield: 52%) as colorless oily liquid.

To a solution of compound 2 (80 mg, 0.21 mmol) in DCM (1 mL) was added TFA (1 mL). The reaction mixture was stirred at 20° C. for 1 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 3 (50 mg, 85%) as colorless oily liquid.

To a solution of compound 3 (119 mg, 0.42 mmol), compound 4 (210 mg, 0.32 mmol), and PyBop (253 mg, 0.49 mmol) in DMF (3 mL) was added DBU (61 mg, 0.49 mmol). The mixture was stirred at 25° C. for 1 h. The solution was extracted with ethyl acetate (80 mL×3). The organic layer was washed with brine (100 mL), dried over anhydrous sodium sulfate, filtered and the filtrate was concentrated to dryness under reduced pressure. The residue was purified by silica gel column (DCM/MeOH=15/1) to give compound 5 (170 mg, Yield: 58%) as a white solid. MS m/z (ESI): 910.2 [M+H]⁺.

To a solution of compound 5 (120 mg, 0.13 mmol) in ethyl acetate (3 ml) was added Pd/C (60 mg). The reaction mixture was stirred at 25° C. under H₂ for 18 min. The reaction mixture was diluted with ethyl acetate (30 ml). The organic layer was filtered, and concentrated to afford compound 6 (90 mg, 89%) as a yellow solid. MS m/z (ESI): 776.2 [M+H]⁺.

To a solution of compound 6 (90 mg, 0.12 mmol) in DCM (2 mL) was added TFA (2 mL). The reaction mixture was stirred at 20° C. for 30 min. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford 232 (70 mg, 89.3%) as colorless oily liquid. MS m/z (ESI): 676.3 [M+H]⁺.

¹H NMR (400 MHz, DMSO-d₆) δ 8.27 (s, 1H), 8.11 (s, 3H), 7.30-7.19 (m, 1H), 7.19-7.09 (m, 1H), 5.27 (d, J=53.6 Hz, 1H), 4.29-3.70 (m, 8H), 3.28-2.58 (m, 7H), 2.16-1.76 (m, 6H).

### Example G: Synthesis of 1,1,1,3,3,3-hexafluoropropan-2-yl 6-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-1,6-diazaspiro[3.4]octane-1-carboxylate (237)

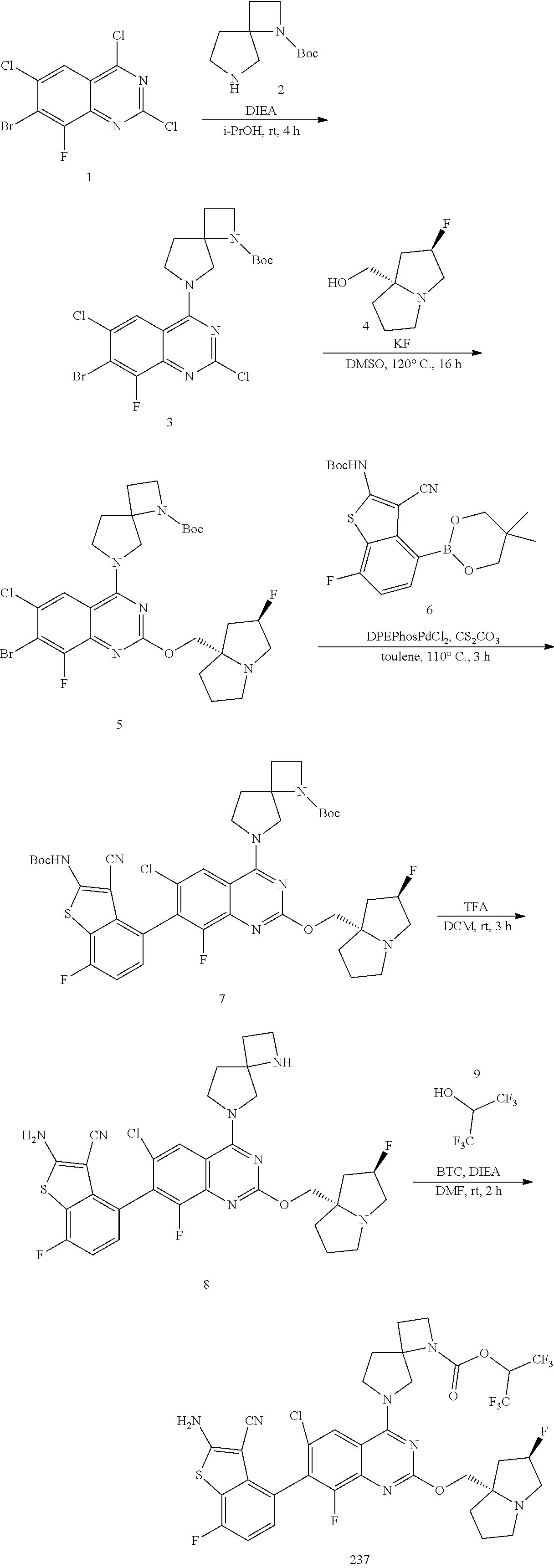

To a solution of compound 1 (1.5 g, 4.54 mmol) and compound 2 (964 mg, 4.54 mmol) in i-PrOH (15 mL) was added DIEA (1.76 g 13.62 mmol). The reaction mixture was stirred at 25° C. for 3 h. The mixture was filtered and the solid was diluted with PE (200 mL). The organic solution was concentrated to dryness under reduced pressure to give compound 3 (2 g, Yield: 87%) as a yellow solid. MS m/z (ESI): 505.1 [M+H]⁺.

To a solution of compound 3 (1 g, 1.98 mmol) and compound 4 (1.57 g, 9.88 mmol) in DMSO (15 mL) was added KF (574 mg, 9.88 mmol). The reaction mixture was stirred at 120° C. for 16 h. The mixture was poured into ice water (100 mL), and the solution was extracted with ethyl acetate (200 mL×3). The organic layer was washed with brine (200 mL), dried over anhydrous sodium sulfate, filtered and the filtrate was concentrated to dryness under reduced pressure. The residue was purified by silica gel column (PE/EA=1/1) to give compound 5 (550 mg, Yield: 44%) as a white solid. LCMS: Rt: 1.416 min; MS m/z (ESI): 628.2 [M+H]⁺.

To a solution of compound 5 (450 mg, 0.72 mmol), compound 6 (434 mg, 1.07 mmol), and Cs₂CO₃ (103 mg, 2.15 mmol) in toluene (10 mL) was added DPEPhosCl₂ (92 mg, 0.14 mmol). The mixture was stirred at 110° C. under nitrogen for 3 hours. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 7 (350 mg, Yield: 58%) as a white solid. MS m/z (ESI): 839.9 [M+H]⁺.

To a solution of compound 7 (350 mg, 0.42 mmol) in DCM (1 mL) was added TFA (1 mL). The reaction mixture was stirred at 25° C. for 1 h. The solvent was removed under reduced pressure. The reaction mixture was diluted with a.q. NaHCO₃ (150 mL) and DCM (50 mL). The organic layer was washed with brine, dried over sodium sulfate, filtered, and concentrated to afford compound 8 (300 mg, 82%) as a yellow solid. MS m/z (ESI): 640.6 [M+H]⁺.

To a solution of compound 9 (526 mg, 3.13 mmol) and DIEA (809 mg, 6.26 mmol) in DCM (10 mL), was added BTC (232 mg, 0.78 mmol) at 0° C. under N₂ atmosphere. The reaction mixture was then stirred at rt for 2 h. To a solution of compound 8 (50 mg, 0.078 mmol) and DIEA (30 mg, 0.234 mmol) in DMF (0.3 mL), was added above reaction mixture (0.5 mL) at 20° C. under N₂ atmosphere. The crude was purified by prep-HPLC to give 237 (11.60 mg, Yield: 17.8%). MS m/z (ESI): 834.4 [M+H]⁺.

¹H NMR (400 MHz, DMSO-d₆) δ 8.46-7.91 (m, 3H), 7.24 (dt, J=8.8, 4.6 Hz, 1H), 7.15 (t, J=8.8 Hz, 1H), 6.71-6.41 (m, 11H), 5.60-5.20 (m, 11H), 4.46-3.89 (m, 8H), 3.27-2.92 (m, 2H), 2.70-2.51 (m, 2H), 2.45-1.53 (m, 1011).

### Example H: Synthesis of(6-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,6-diazaspiro[3.4]octan-1-yl)(1H-1,2,4-triazol-1-yl)methanone (277)

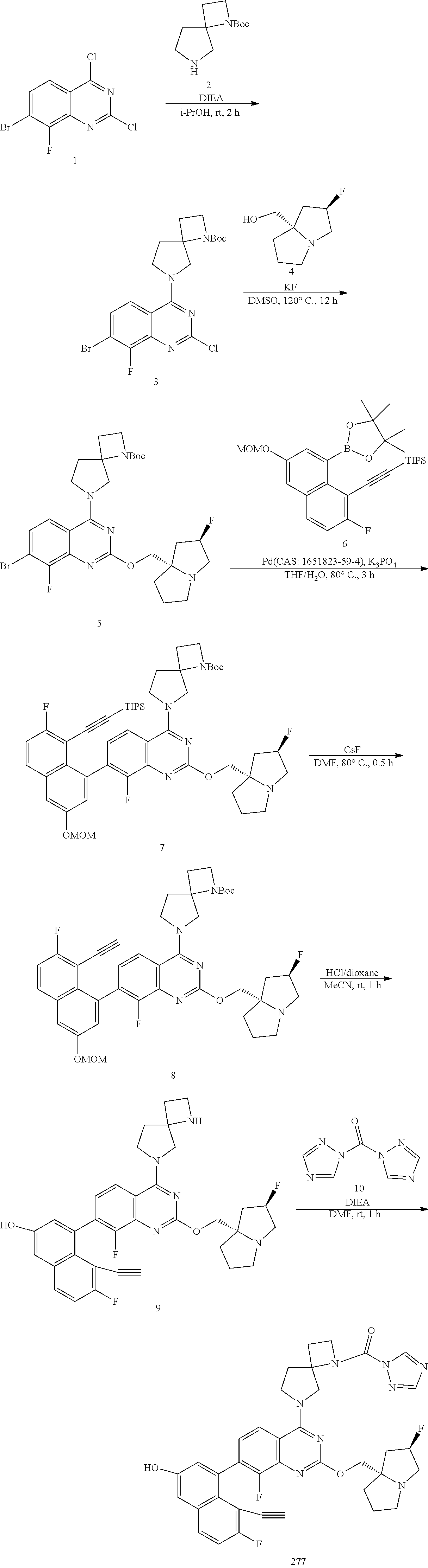

To a solution of compound 2 (717.54 mg, 3.38 mmol) and N,N-Diisopropylethylamine (1.31 g, 10.14 mmol) in dry dioxane (10 mL) was added compound 1 (1 g, 3.38 mmol). The reaction was stirred at room temperature for 2 hr. The mixture was poured into ice water (100 mL). The mixture was filtered and the solid was collected to obtain compound 3 as a yellow solid (1.3 g, Yield: 81.7%). MS m/z (ESI): 471.1 [M+H]⁺.

To a solution of compound 3 (1.2 g, 2.54 mmol) and KF (736.6 mg, 12.7 mmol) in DMSO (15 mL) was added compound 4 (2 g, 12.7 mmol). The reaction mixture was stirred at 120° C. for 12 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=20/1) to give compound 5 (639 mg, Yield: 42.6%) as a white solid. MS m/z (ESI): 596.2 [M+H]⁺.

To a solution of compound 5 (570 mg, 0.96 mmol), compound 6 (588 mg, 1.15 mmol) and K₃PO₄ (607 mg, 2.86 mmol) in dry THF/H₂O (8 mL/2 mL) was added calcium A Pd G3 (69 mg, 0.095 mmol). The mixture was stirred at 80° C. for 3 hr under N₂. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (EA:MeOH=20:1) to give compound 7 (750 mg, Yield: 72.5%) as a white solid. MS m/z (ESI): 901.4 [M+H]⁺.

To a solution of compound 7 (240 mg, 0.267 mmol) in dry DMF (45 mL) was added CsF (405.48 mg, 2.67 mmol). The mixture was stirred at 60° C. for 0.5 hr. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure to give compound 8 (188.6 mg, Yield: 95%) as a white solid. MS m/z (ESI): 744.8 [M+H]⁺.

To a solution of compound 8 (188.6 mg, 0.25 mmol) in CH₃CN (5 mL) was added HCl/dioxane (3 mL, 12 mmol). The solution was stirred at 20° C. for 1 h. Then the solution was added dropwise to n-hexane (50 mL), the upper clear liquid was removed and the residue was dissolved in a solution of DCM (100 mL) and MeOH (10 mL). The organic layer was washed by aqueous a.q. NaHCO₃ (50 mL), and brine (50 mL). The organic layer was separated, dried over Na₂SO₄, concentrated to give compound 9 (144.48 mg, Yield: 95%) as a yellow solid. MS m/z (ESI): 600.4 [M+H]⁺.

To a solution of compound 9 (40 mg, 0.067 mmol) and DIEA (25.8 mg, 0.2 mmol) in DMF (2 mL) was added compound 10 (27.4 mg, 0.167 mmol) at 0° C. under N₂. The mixture was stirred at room temperature for 1h. The mixture was concentrated and purified by prep-HPLC (FA) to give 277 (13.35 mg, Yield: 28.8%) as a white solid.

¹H NMR (400 MHz, DMSO-d⁶) δ 10.72-9.76 (s, 1H), 9.16 (s, 1H), 8.28 (s, 1H), 8.24 (s, 1H), 8.08-7.92 (m, 2H), 7.48-7.32 (m, 2H), 7.24-7.04 (m, 2H), 5.28 (d, J=54.0 Hz, 1H), 4.72-4.52 (m, 3H), 4.20 (s, 2H), 4.12-3.96 (m, 3H), 3.87 (d, J=36.8 Hz, 1H), 3.12-3.00 (m, 4H), 2.92-2.80 (m, 2H), 2.67 (s, 1H), 2.33 (s, 1H), 2.12-2.00 (m, 3H), 1.88-1.72 (m, 3H).

### Example I: Synthesis of 4-(4-(1-(1H-1,2,4-triazole-1-carbonyl)-1,7-diazaspiro[4.5]decan-7-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (246)

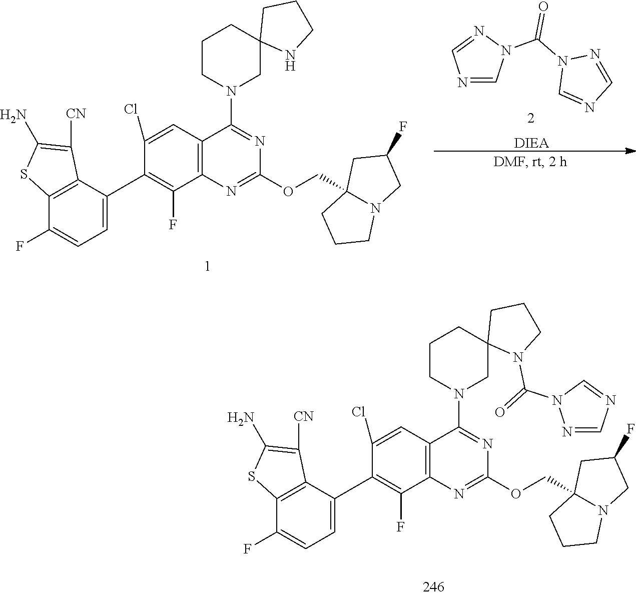 [SMILES: N#Cc1c(N)sc2c(F)ccc(-c3c(Cl)cc4c(N5CCCC6(CCCN6)C5)nc(OC[C@@]56CCCN5C[C@H](F)C6)nc4c3F)c12 ; O=C(n1cncn1)n1cncn1 ; N#Cc1c(N)sc2c(F)ccc(-c3c(Cl)cc4c(N5CCCC6(CCCN6C(=O)n6cncn6)C5)nc(OC[C@@]56CCCN5C[C@H](F)C6)nc4c3F)c12]

To a solution of compound 1 (50 mg, 0.075 mmol) and DIEA (29 mg, 0.225 mmol) in DMF (2 mL), was added compound 2 (37 mg, 0.225 mmol). The reaction mixture was then stirred at rt for 2 h. LCMS showed the reaction was completed. The crude was purified by prep-HPLC to give 246 (1.57 mg, Yield: 2.8%). MS m/z (ESI): 763.24 [M+H]⁺.

¹H NMR (400 MHz, DMSO-d₆) δ 9.09 (s, 1H), 8.21 (s, 1H), 8.11 (s, 2H), 7.87 (s, 1H), 7.30-7.24 (m, 1H), 7.17-7.13 (m, 1H), 5.27 (d, J=54.0 Hz, 1H), 4.33-3.75 (m, 6H), 3.24-3.15 (m, 3H), 3.13-2.90 (m, 3H), 2.83-2.75 (m, 2H), 2.20-1.60 (m, 12H).

### Example J: Synthesis of 4-(4-(1-(1H-1,2,3-triazole-1-carbonyl)-1,6-diazaspiro[3.4]octan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (248)

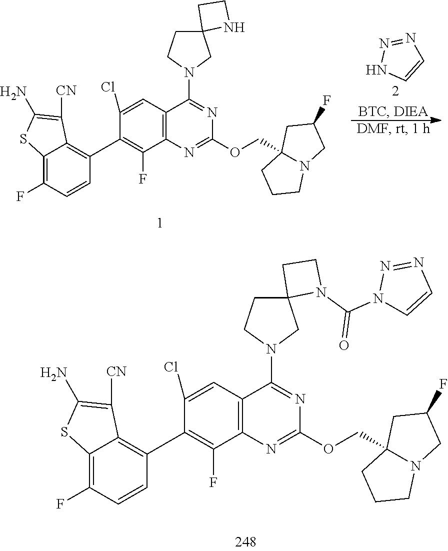 [SMILES: N#Cc1c(N)sc2c(F)ccc(-c3c(Cl)cc4c(N5CCC6(CCN6)C5)nc(OC[C@@]56CCCN5C[C@H](F)C6)nc4c3F)c12 ; c1c[nH]nn1 ; N#Cc1c(N)sc2c(F)ccc(-c3c(Cl)cc4c(N5CCC6(CCN6C(=O)n6ccnn6)C5)nc(OC[C@@]56CCCN5C[C@H](F)C6)nc4c3F)c12]

To a solution of compound 2 (432 mg, 6.26 mmol) and DIEA (1.21 g, 9.39 mmol) in MeCN (10 mL), was added BTC (1.67 g, 5.63 mmol) at 0° C. under N₂ atmosphere. The reaction mixture was then stirred at rt for 1 h. To a solution of compound 1 (40 mg, 0.0626 mmol) in DMF (0.5 mL), was added above reaction mixture (0.1 mL) at rt under N₂ atmosphere. LCMS showed the reaction was completed. The crude was purified by prep-HPLC to give 248 (8.25 mg, Yield: 18%). MS m/z (ESI): 735.2 [M+H]⁺.

¹H NMR (400 MHz, DMSO-d₆) δ 8.16-8.11 (m, 5H), 7.90 (m, 1H), 7.27-7.21 (m, 1H), 7.17-7.13 (m, 1H), 5.27 (d, J=53.2 Hz, 1H), 4.76-4.47 (m, 3H), 4.38-3.92 (m, 6H), 3.14-3.07 (m, 2H), 3.01 (s, 1H), 2.83-2.81 (m, 2H), 2.67-2.59 (m, 1H), 2.37 (s, 2H), 2.14-2.02 (m, 3H), 1.84-1.76 (m, 3H).

### Example K: Synthesis of 2-amino-4-(6-chloro-8-fluoro-2-(((2R,7aS)-2-fluorotetrahydro-1H-pyrrolizin-7a(5H)-yl)methoxy)-4-(1,7-diazaspiro[4.4]nonan-7-yl)quinazolin-7-yl)-7-fluorobenzo[b]thiophene-3-carbonitrile (284)

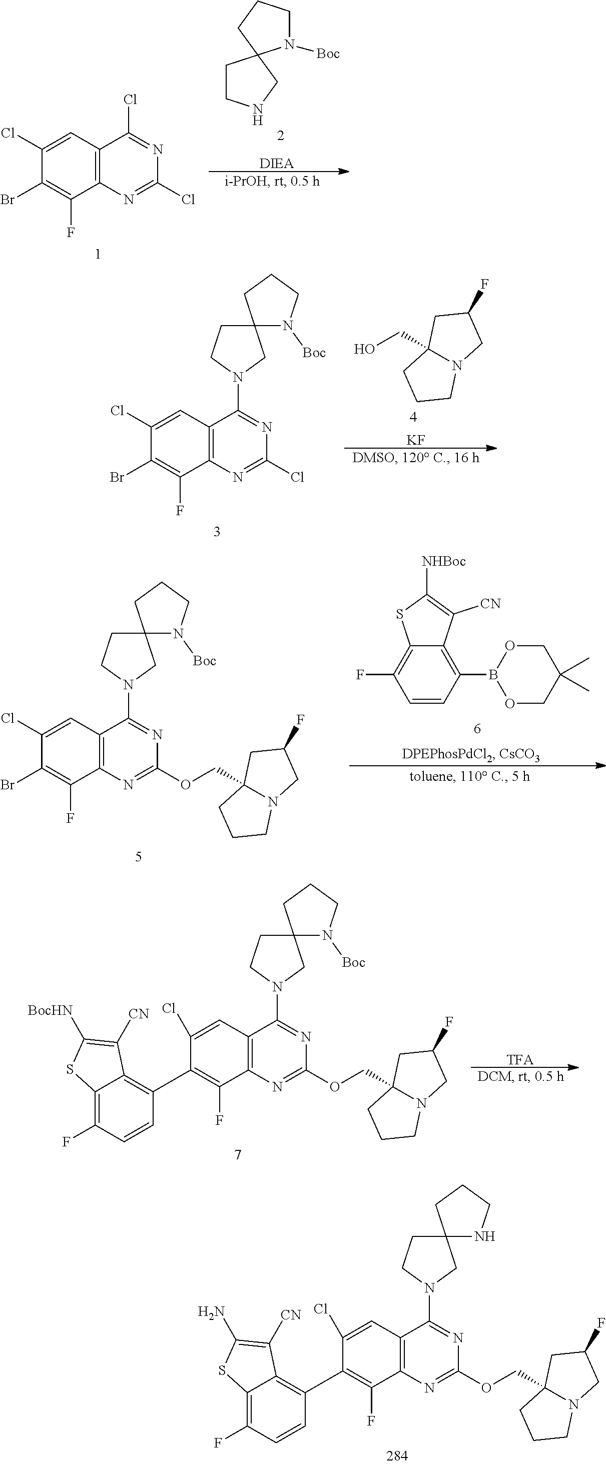

To a solution of compound 1 (450 mg, 1.37 mmol) and compound 2 (310 mg, 1.37 mmol) in i-PrOH (15 mL) was added DIEA (530 mg, 4.11 mmol). The reaction mixture was stirred at 25° C. for 0.5 h. LCMS showed the reaction was completed. The mixture was poured into ice water (200 mL). The mixture was filtered and the solid was diluted with PE (150 mL). The organic solution was concentrated to dryness under reduced pressure to give compound 3 (450 mg, Yield: 63%) as a white solid. MS m/z (ESI): 518.0 [M+H]⁺.

To a solution of compound 3 (450 mg, 0.87 mmol) and compound 4 (684 mg, 4.35 mmol) and KF (252 mg, 4.35 mmol) in DMSO (10 mL). The reaction mixture was then stirred at 120° C. for 16 h. LCMS showed the reaction was completed. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The crude was purified by SGC (PE/EA=1/1) to give compound 5 (110 mg, Yield: 20%) as a yellow solid. MS m/z (ESI): 644.2 [M+H]⁺.

To a solution of compound 5 (110 mg, 0.17 mmol), compound 6 (110 mg, 0.26 mmol), and Cs₂CO₃(176 mg, 0.51 mmol) in toluene (7 mL) was added DPEPhosPdCl₂ (33 mg, 0.03 mmol). The mixture was stirred at 110° C. under nitrogen for 5 hours. LCMS showed the reaction was completed. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The crude was purified by SGC (PE/EA=1/1) to give compound 7 (85 mg, Yield: 58%) as a white solid. MS m/z (ESI): 854.4 [M+H]⁺.

To a solution of compound 7 (20 mg, 0.02 mmol) in DCM (0.5 mL) was added TFA (0.5 mL). The reaction mixture was stirred at r.t for 0.5 h. LCMS showed the reaction was completed. The reaction mixture removed under the reduced pressure. The residue was diluted with dichloromethane (100 mL). The solution was basified with NaHCO₃. The organic layer was washed with brine, dried over sodium sulfate, filtered, and concentrated to dryness under reduced pressure to give compound 284 (12.18 mg, Yield: 75.9%) as a white solid. MS m/z (ESI): 654.3 [M+H]⁺.

¹H NMR (400 MHz, DMSO-d₆) δ 8.13 (s, 1H), 8.12 (s, 3H), 7.24-7.13 (m, 2H), 5.36 (d, J=53.6 Hz, 1H), 4.26 (t, J=11.6 Hz, 2H), 4.11 (s, 4H), 3.34 (s, 3H), 3.13-2.71 (m, 2H), 2.43-2.39 (m, 1H), 2.35-1.75 (m, 12H).

### Example L: Synthesis of 2-amino-4-((R)-6-chloro-4-((R)-1-(3-chloro-1H-1,2,4-triazole-1-carbonyl)-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (225)

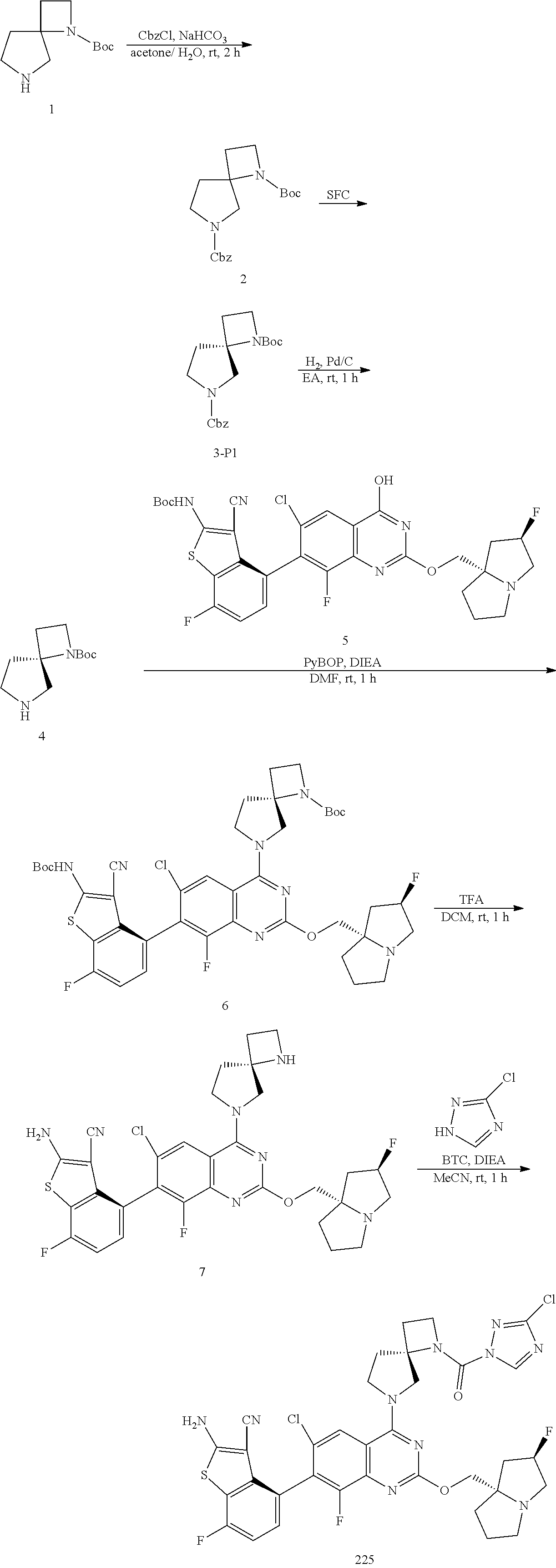

To a solution of compound 1 (2 g, 3.89 mmol) and NaHCO₃ (1.31 g, 15.56 mmol) in acetone (20 mL) was added H₂O (10 mL). The reaction mixture was added CbzCl (1.33 g, 7.78 mmol) stirred at 25° C. for 1 h. The solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=2/1) to give compound 2 (4 g, Yield: 61%) as colorless oily liquid.

¹H NMR (400 MHz, CDCl₃) δ 7.40-7.26 (m, 5H), 5.40-4.94 (m, 2H), 4.22-3.12 (m, 6H), 2.54 (d, J=73.8 Hz, 1H), 2.31-2.04 (m, 2H), 1.96 (s, 1H), 1.55-1.23 (m, 911).

Compound 2 (4 g, 11.55 mmol) was separated by SFC (solvent: EtOH) to give compound 3-P1 (1.8 g) as colorless oily liquid and compound 3-P2 (1.6 g) as colorless oily liquid.

To a solution of compound 3-P1 (1.5 g, 4.33 mmol) in ethyl acetate (50 mL) was added Pd/C (750 mg). The reaction mixture was stirred at 25° C. under H₂ atmosphere for 1 h. The reaction mixture was diluted with ethyl acetate (50 mL) and methanol (100 mL). The organic layer was filtered, and concentrated to afford compound 4 (780 mg, 85%) as colorless oily liquid.

¹H NMR (400 MHz, CDCl₃) δ 4.04-3.65 (m, 2H), 3.54-3.05 (m, 2H), 2.95-2.62 (m, 2H), 2.42-2.17 (m, 4H), 1.91 (ddd, J=13.4, 8.2, 4.8 Hz, 1H), 1.65-1.35 (m, 911).

To a solution of compound 4 (299 mg, 1.41 mmol), compound 5 (700 mg, 1.09 mmol), and PyBop (847 mg, 1.63 mmol) in DMF (5 mL) was added DIEA (210 mg, 1.63 mmol). The mixture was stirred at 25° C. for 1 h. The solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=15/1) to give compound 6 (700 mg, Yield: 77%) as a yellow solid. MS m/z (ESI): 840.3 [M+H]⁺.

To a solution of compound 6 (1.3 g, 1.55 mmol) in DCM (5 mL) was added TFA (5 mL). The reaction mixture was stirred at 20° C. for 30 min. The solvent was removed under reduced pressure. The reaction mixture was diluted with a.q. NaHCO₃ (200 mL) and DCM/MeOH=10/1 (300 mL). The organic layer was washed with brine, dried over sodium sulfate, filtered, and concentrated to give compound 7 (830 mg, Yield: 84%) as a yellow solid. MS m/z (ESI): 640.6 [M+H]⁺.

To a solution of compound 8 (516 mg, 5.006 mmol) and DIEA (2.59 g, 20.024 mmol) in MeCN (17 mL), was added BTC (1337 mg, 4.505 mmol) at 0° C. under N₂ atmosphere. The reaction mixture was then stirred at rt for 2 h. To a solution of compound 7 (160 mg, 0.25 mmol) and DIEA (97 mg, 0.75 mmol) in DMF (2 mL), was added above reaction mixture (0.16 mL) at rt under N₂ atmosphere. LCMS showed the reaction was completed. The crude was purified by prep-HPLC to give 225 (15.83 mg, Yield: 8.2%). MS m/z (ESI): 769.1 [M+H]⁺.

¹H NMR (400 MHz, DMSO-d₆) δ 9.24 (s, 1H), 8.21 (s, 1H), 8.12 (d, J=16.6 Hz, 3H), 7.23 (m, 1H), 7.15 (t, J=8.8 Hz, 1H), 5.27 (d, J=53.8 Hz, 1H), 4.72-3.96 (m, 8H), 3.15-2.63 (m, 6H), 2.32-2.11 (m, 2H), 2.12-1.74 (m, 6H).

### Example M: Synthesis of 2-amino-4-(6-chloro-4-(1-((2R,3S)-3-cyclopropylaziridine-2-carbonyl)-1,7-diazaspiro[3.5]nonan-7-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Compound 262)

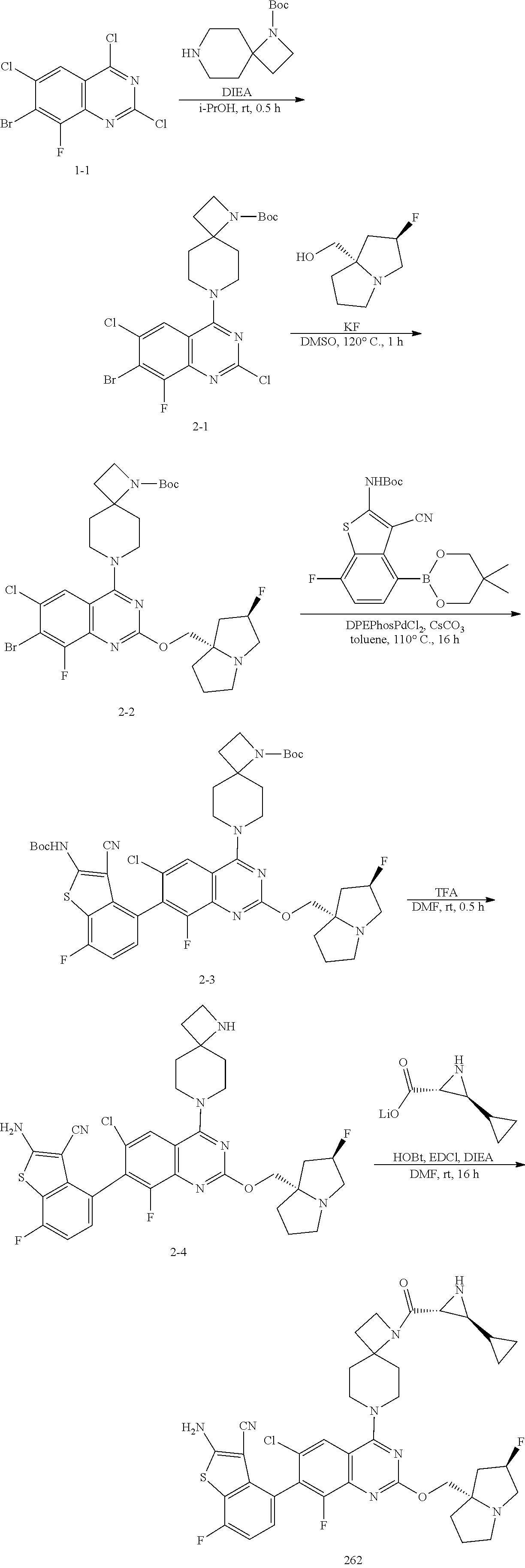

To a solution of compound 1-1 (1.4 g, 4.28 mmol) and tert-butyl 1,7-diazaspiro[3.5]nonane-1-carboxylate (967 mg, 4.28 mmol) in i-PrOH (10 ml) was added DIEA (1.6 g 12.84 mmol). The reaction mixture was stirred at 25° C. for 0.5 h. The mixture was poured into ice water (200 mL). The mixture was filtered and the solid was diluted with MeOH (150 ml). The organic solution was concentrated to dryness under reduced pressure to give compound 2-1 (2 g). ESI-MS m/z: 521.1 [M+H]⁺.

To a solution of compound 2-1 (2 g, 3.86 mmol) and ((2R,7aS)-2-fluorotetrahydro-1H-pyrrolizin-7a(5H)-yl)methanol (3.1 g, 19.3 mmol) in DMSO (10 mL) was added KF (1.2 g, 19.3 mmol). The reaction mixture was stirred at 120° C. for 1 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 2-2 (1.0 g). ESI-MS m/z: 644.2 [M+H]⁺.

To a solution of compound 2-2 (320 mg, 0.50 mmol), tert-butyl (3-cyano-4-(5,5-dimethyl-1,3,2-dioxaborinan-2-yl)-7-fluorobenzo[b]thiophen-2-yl)carbamate (303 mg, 0.75 mmol), and Cs₂CO₃ (487 mg, 1.50 mmol) in toluene (10 mL) was added DPEPhosPdCl₂ (72 mg, 0.10 mmol). The mixture was stirred at 110° C. under nitrogen for 16 hours. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 2-3 (290 mg). ESI-MS m/z: 854.3 [M+H]⁺.

To a solution of compound 2-3 (290 mg, 0.34 mmol) in DCM (2 mL) was added TFA (1 mL). The reaction mixture was stirred at 25° C. for 0.5 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 2-4 (140 mg). ESI-MS m/z: 654.1 [M+H]⁺.

A solution of compound 2-4 (25 mg, 0.038 mmol), lithium (2R,3S)-3-cyclopropylaziridine-2-carboxylate (15 mg, 0.115 mmol), HOBt (16 mg, 0.115 mmol), EDCI (22 mg, 0.115 mmol) and DIEA (15 mg, 0.115 mmol) in DMF (3 mL) was stirred at RT for 16 h. When LCMS showed the reaction was completed. The reaction solution was washed with water, extracted with EA. The organic phase was dried by anhydrous Na₂SO₄, concentrated to give the residue under reduced pressure. The crude product was purified by prep-HPLC to give compound 262 (2.85 mg). ESI-MS m/z: 763.6 [M+H]⁺; ¹H NMR (400 MHz, DMSO-d₆): δ 8.10 (s, 2H), 7.90-7.76 (m, 1H), 7.26 (dd, J=8.2, 5.2 Hz, 1H), 7.14 (t, J=9.0 Hz, 1H), 5.27 (d, J=54.4 Hz, 1H), 4.50-3.75 (m, 8H), 3.30-3.19 (m, 2H), 3.15-2.95 (m, 3H), 2.85-2.79 (m, 1H), 2.46-2.41 (m, 1H), 2.33-2.25 (m, 2H), 2.23-1.85 (m, 7H), 1.80-1.70 (m, 2H), 1.25-1.10 (m, 1H), 0.95-0.75 (m, 1H), 0.42-0.19 (m, 4H).

### Example N: Synthesis of 2-amino-4-(6-chloro-8-fluoro-4-(1-(1-methyl-1H-pyrazole-5-carbonyl)-1,6-diazaspiro[3.5]nonan-6-yl)-2-((S)-1-((S)-1-methylpyrrolidin-2-yl)ethoxy)quinazolin-7-yl)-7-fluorobenzo[b]thiophene-3-carbonitrile (282)

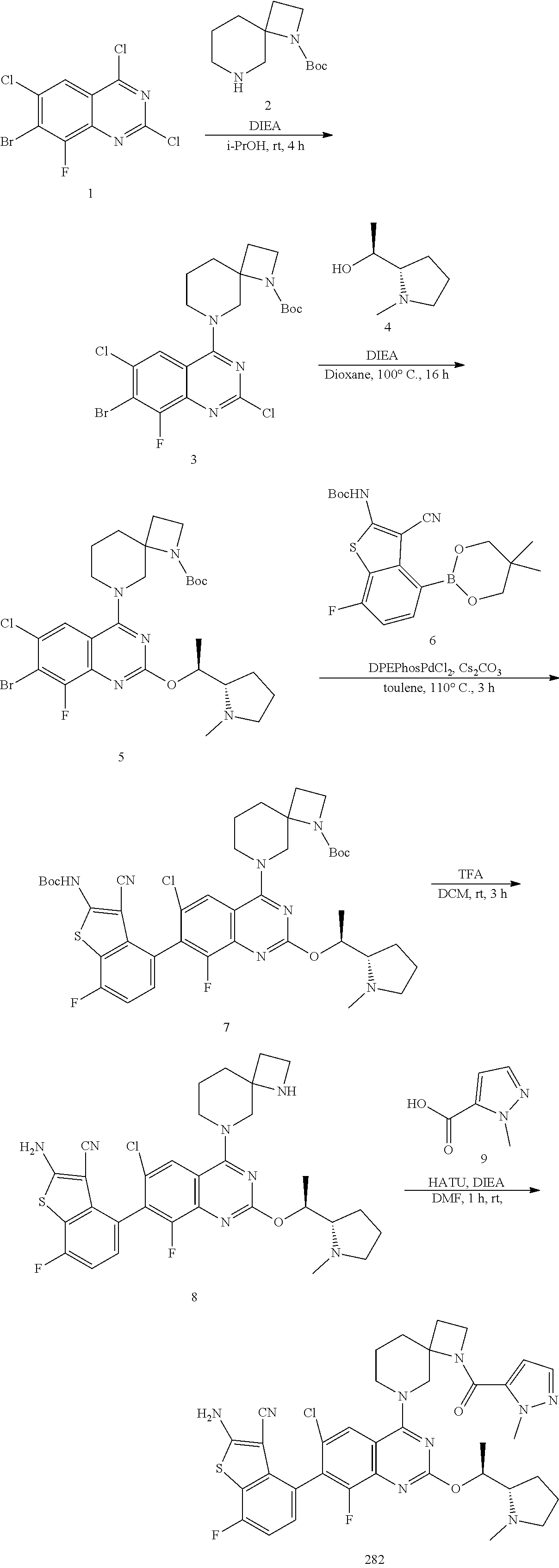

To a solution of compound 1 (1.0 g, 3.03 mmol) and compound 2 (685 mg, 3.03 mmol) in i-PrOH (8 mL) was added DIEA (1.17 g, 9.09 mmol). The reaction mixture was stirred at 25° C. for 4 h. The mixture was poured into ice water (200 mL). The mixture was filtered and the solid was diluted with MeOH (150 mL). The organic solution was concentrated to dryness under reduced pressure to give compound 3 (1.3 g, Yield: 82.8%) as a yellow solid. MS m/z (ESI): 521.0 [M+H]⁺.

To a solution of compound 3 (1.3 g, 2.50 mmol) and compound 4 (1.61 g, 12.49 mmol) in DMSO (10 mL) was added KF (724 mg, 12.49 mmol). The reaction mixture was stirred at 120° C. for 16 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 5 (500 mg, Yield: 32.7%) as a yellow solid. MS m/z (ESI): 611.9 [M+H]⁺.

To a solution of compound 5 (500 mg, 0.82 mmol), compound 6 (413 mg, 1.23 mmol), and Cs₂CO₃ (802 mg, 2.46 mmol) in toluene (15 mL) was added DPEPhosPdCl₂ (117 mg, 0.16 mmol). The mixture was stirred at 110° C. under nitrogen for 3 hours. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 7 (290 mg, Yield: 42.9%) as a white solid. MS m/z (ESI): 824.4 [M+H]⁺.

To a solution of compound 7 (290 mg, 0.35 mmol) in DCM (3 mL) was added TFA (2 mL). The reaction mixture was stirred at 20° C. for 3 h. The solvent was removed under reduced pressure. The reaction mixture was diluted with a.q. NaHCO₃ (30 mL) and DCM (100 mL). The organic layer was washed with brine, dried over sodium sulfate, filtered, and the residue was purified by work up to give compound 8 (200 mg, Yield: 91.3%) as a yellow solid. MS m/z (ESI): 624.3 [M+H]⁺.

To a solution of compound 9 (80 mg, 0.64 mmol) and DIEA (165 mg, 1.28 mmol) in DMF (10 mL) was added HATU (243 mg, 0.64 mmol) at 0° C. The reaction mixture was then stirred at 20° C. for 30 min. To a solution of compound 8 (40 mg, 0.064 mmol) in DMF (1 mL) was added above reaction mixture (1 mL). The mixture was stirred at 25° C. for 30 min. The reaction was purified by prep-HPLC (FA) to give 282 (3.01 mg, Yield: 6.41%) as a white solid.

¹H NMR (400 MHz, DMSO-d₆) δ 8.11 (s, 2H), 7.86 (d, J=15.6 Hz, 1H), 7.49 (s, 1H), 7.33-7.23 (m, 1H), 7.16 (t, J=8.8 Hz, 1H), 6.64 (d, J=13.2 Hz, 1H), 5.28 (d, J=5.6 Hz, 1H), 4.59-4.39 (m, 1H), 4.26-4.05 (m, 3H), 4.01 (s, 3H), 3.29-3.10 (m, 2H), 2.94 (t, J=6.4 Hz, 1H), 2.62 (d, J=6.8 Hz, 2H), 2.36 (d, J=3.2 Hz, 3H), 2.20-1.95 (m, 4H), 1.87 (s, 2H), 1.79-1.70 (m, 2H), 1.68-1.57 (m, 2H), 1.25 (t, J=6.4 Hz, 3H). MS m/z (ESI): 732.3 [M+H]⁺.

### Example O: Synthesis of 4-(4-(1-(1H-1,2,4-triazole-1-carbonyl)-1,6-diazaspiro[3.4]octan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (Compound 272)

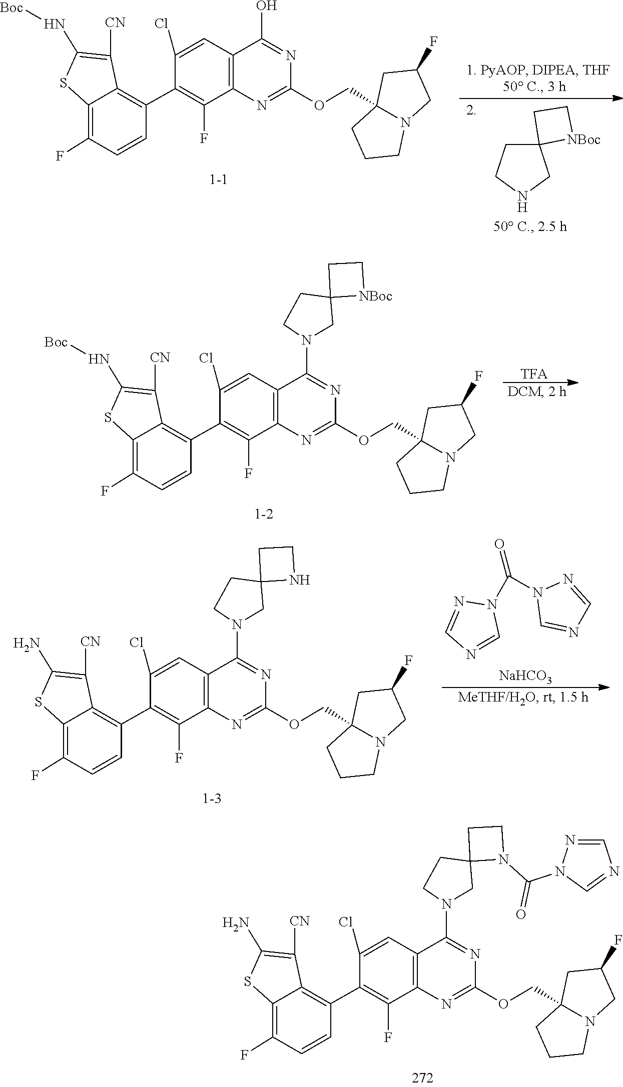

To a solution of compound 1-1 (102 mg, 0.16 mmol) in THF (7 ml), was added DIPEA (200 uL, 1.15 mmol) and PyAOP (212 mg, 0.41 mmol) solution in 0.5 ml of DMF. The reaction mixture was stirred at 50° C. for 3 h. Then tert-butyl 1,6-diazaspiro[3.4]octane-1-carboxylate (200 mg, 0.94 mmol) solution in THF (2 mL) and DIPEA (1 mL, 5.75 mmol) were added into the reaction mixture. After stirring at 50° C. for 2.5 h, it was concentrated and purified by silica gel column (Hexane/EA=1/1) to give compound 1-2 (160 mg). ESI-MS m/z: 840.5 [M+H]⁺.

To a solution of compound 1-2 (160 mg, 0.19 mmol) in DCM (7 mL) was added TFA (1 mL). The reaction mixture was stirred at room temperature for 2 h. The solvent was removed under reduced pressure. The reaction mixture was diluted with a.q. NaHCO₃ and DCM. The organic layer was washed with brine, dried over sodium sulfate, filtered, and concentrated to afford compound 1-3 (121 mg). ESI-MS m/z: 640.4 [M+H]⁺.

To a solution of compound 1-3 (121 mg, 0.19 mmol) in MeTHF/water (5 ml/5 ml) was added NaHCO₃ (200 mg, 2.38 mmol) and di(1H-1,2,4-triazol-1-yl)methanone (24 mg, 0.150 mmol). The reaction mixture was stirred at room temperature for 1.5 h and was washed with water, extracted with EA. The organic phase was dried by anhydrous Na₂SO₄, and concentrated to give the residue under reduced pressure. The crude product was purified by prep-HPLC to give compound 272 (3.8 mg). ESI-MS m/z: 735.4 [M+H]⁺.

### Example P: Synthesis of 2-amino-4-((R)-6-chloro-4-((R)-1-(3-(difluoromethyl)-1H-1,2,4-triazole-1-carbonyl)-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (278)

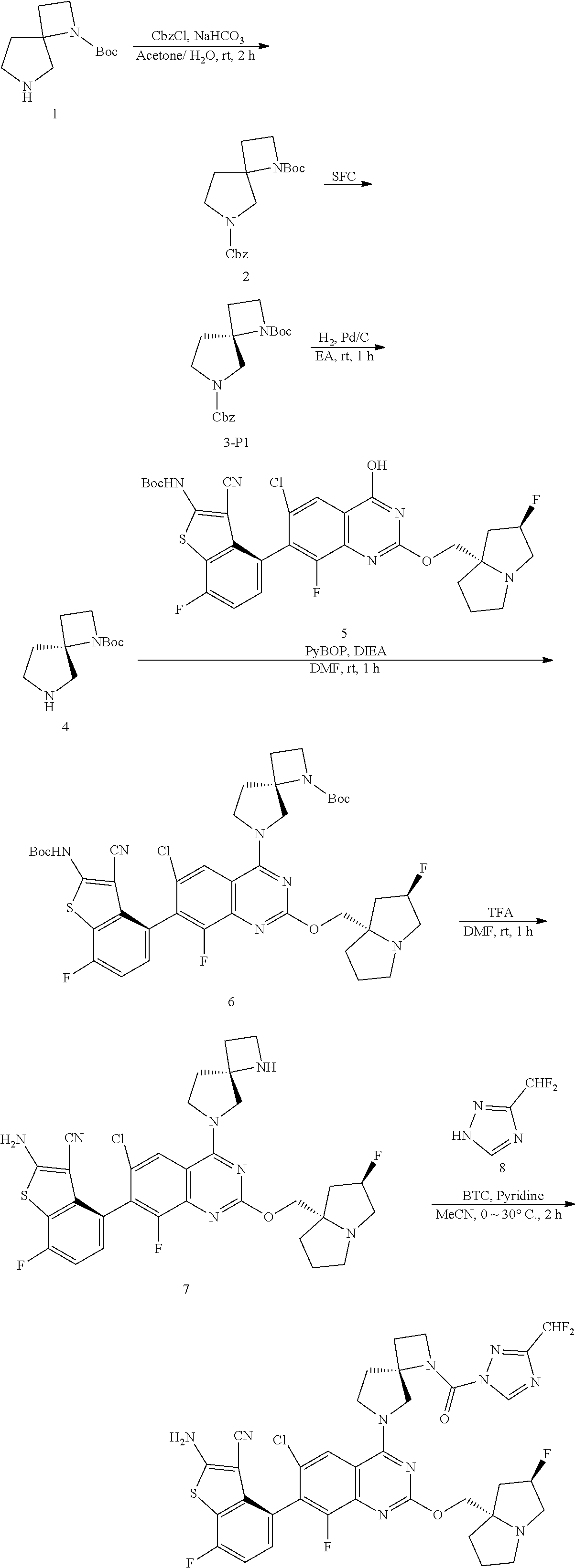

To a solution of compound 1 (2 g, 7.78 mmol) and NaHCO₃ (1.31 g, 15.56 mmol) in acetone (20 ml) and H₂O (10 ml) was added CbzCl (1.33 g, 7.78 mmol). The reaction mixture was stirred at 25° C. for 1 h. The solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=2/1) to give compound 2 (2.2 g, Yield: 81%) as colorless oily liquid.

¹H NMR (400 MHz, CDCl₃) δ 7.40-7.26 (m, 5H), 5.20-5.05 (m, 2H), 4.00-3.30 (m, 6H), 2.70-2.30 (m, 1H), 2.25-2.04 (m, 2H), 1.96 (s, 1H), 1.55-1.23 (m, 911).

Compound 2 (4 g, 11.55 mmol) was separated by SFC (solvent: EtOH) to give compound 3-P1 (1.8 g) as colorless oily liquid and compound 3-P2 (1.6 g) as colorless oily liquid.

To a solution of compound 3-P1 (1.5 g, 4.33 mmol) in ethyl acetate (50 ml) was added Pd/C (750 mg). The reaction mixture was stirred at 25° C. under H₂ atmosphere for 1 h. The reaction mixture was diluted with ethyl acetate (50 ml) and methanol (100 ml). The organic layer was filtered, and concentrated to afford compound 4 (780 mg, 85%) as colorless oily liquid.

¹H NMR (400 MHz, CDCl₃) δ 4.04-3.65 (m, 2H), 3.54-3.05 (m, 2H), 2.95-2.62 (m, 2H), 2.42-2.17 (m, 4H), 1.91 (m, 1H), 1.65-1.35 (m, 911).

To a solution of compound 4 (299 mg, 1.41 mmol), compound 5 (700 mg, 1.09 mmol), and PyBop (847 mg, 1.63 mmol) in DMF (5 mL) was added DIEA (210 mg, 1.63 mmol). The mixture was stirred at 25° C. for 1 h. The solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=15/1) to give compound 6 (700 mg, Yield: 77%) as a yellow solid. MS m/z (ESI): 840.3 [M+H]⁺.

To a solution of compound 6 (1.3 g, 1.55 mmol) in DCM (15 ml) was added TFA (15 ml). The reaction mixture was stirred at 20° C. for 30 min. The solvent was removed under reduced pressure. The reaction mixture was diluted with a.q. NaHCO₃ (200 mL) and DCM/MeOH=10/1(300 mL). The organic layer was washed with brine, dried over sodium sulfate, filtered, and concentrated to give compound 7 (830 mg, Yield: 84%) as a yellow solid. MS m/z (ESI): 640.6 [M+H]⁺.

To a solution of compound 8 (274 mg, 2.30 mmol) and pyridine (0.74 mL, 9.21 mmol) in 9 mL MeCN, was added BTC (615 mg, 2.07 mmol) at 0° C. under N₂ atmosphere. The reaction mixture was then stirred at 0° C. for 2 h. To a solution of compound 7 (90 mg, 0.14 mmol) in DME (1 mL) was added pyridine (0.37 ml, 4.6 mmol) and above reaction mixture (0.7 mL) at 30° C. under N₂ atmosphere. LCMS showed the reaction was completed. The mixture was poured into water (15 mL), and the solution was extracted with ethyl acetate (15 mL×3). The organic layer was washed with brine (15 mL), dried over anhydrous sodium sulfate, filtered and the filtrate was concentrated to dryness under reduced pressure. The crude was purified by prep-HPLC (FA) to give final product (27.10 mg, Yield: 24.6%). MS m/z (ESI): 785.2 [M+H]⁺.

1H NMR (400 MHz, DMSO-d₆) δ 9.32 (s, 1H), 8.17-8.11 (m, 3H), 7.39-7.02 (m, 3H), 5.27 (d, J=54.0 Hz, 1H), 4.74-4.49 (m, 3H), 4.27-3.96 (m, 5H), 3.11-3.00 (m, 3H), 2.95-2.75 (m, 2H), 2.67-2.57 (m, 1H), 2.46-2.32 (m, 2H), 2.14-1.71 (m, 6H).

### Example Q: Synthesis of 4-(4-(1-(1H-1,2,4-triazole-1-carbonyl)-1,7-diazaspiro[4.4]nonan-7-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (261)

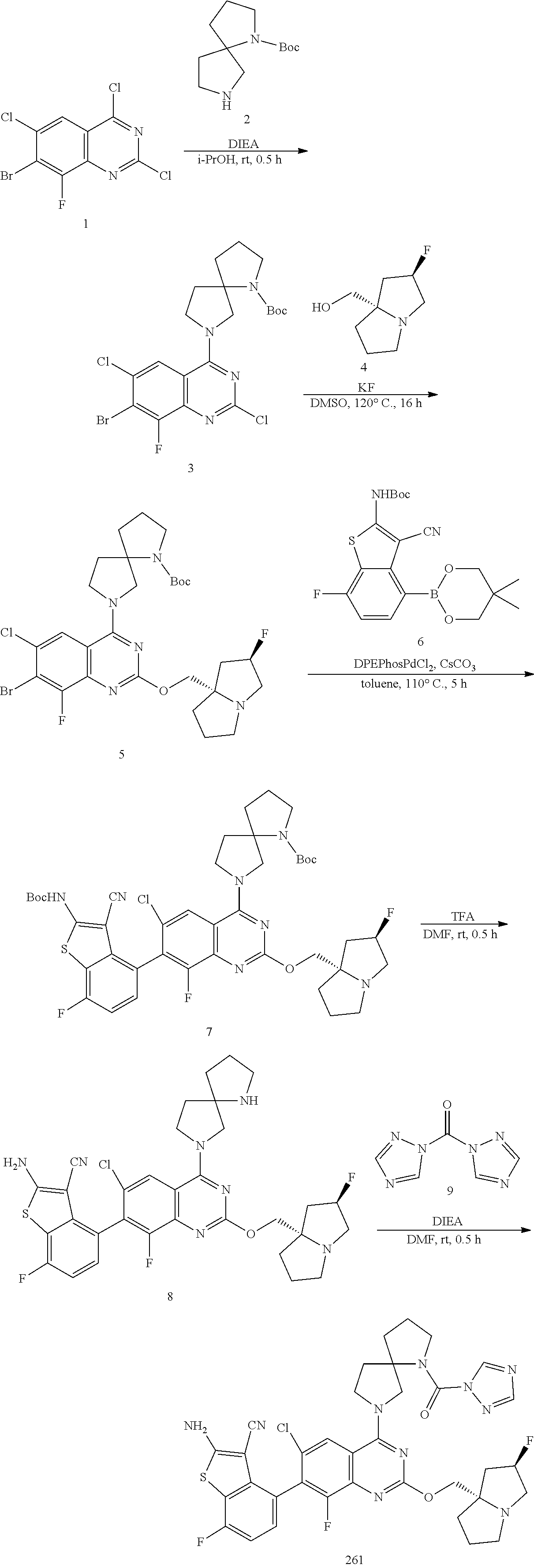

To a solution of compound 1 (450 mg, 1.37 mmol) and compound 2 (310 mg, 1.37 mmol) in i-PrOH (15 mL) was added DIEA (530 mg, 4.11 mmol). The reaction mixture was stirred at 25° C. for 0.5 h. LCMS showed the reaction was completed. The mixture was poured into ice water (200 mL). The mixture was filtered and the solid was diluted with PE (150 mL). The organic solution was concentrated to dryness under reduced pressure to give compound 3 (450 mg, Yield: 63%) as a white solid. MS m/z (ESI): 518.0 [M+H]⁺.

To a solution of compound 3 (450 mg, 0.87 mmol) and compound 4 (684 mg, 4.35 mmol) and KF (252 mg, 4.35 mmol) in DMSO (10 mL). The reaction mixture was then stirred at 120° C. for 16 h. LCMS showed the reaction was completed. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The crude was purified by SGC (PE/EA=1/1) to give compound 5 (110 mg, Yield: 20%) as a yellow solid. MS m/z (ESI): 644.2 [M+H]⁺.

To a solution of compound 5 (110 mg, 0.17 mmol), compound 6 (110 mg, 0.26 mmol), and Cs₂CO₃ (176 mg, 0.51 mmol) in toluene (7 mL) was added DPEPhosPdCl₂ (33 mg, 0.03 mmol). The mixture was stirred at 110° C. under nitrogen for 5 hours. LCMS showed the reaction was completed. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The crude was purified by SGC (PE/EA=1/1) to give compound 7 (85 mg, Yield: 58%) as a white solid. MS m/z (ESI): 854.4 [M+H]⁺.

To a solution of compound 7 (85 mg, 0.10 mmol) in DCM (1 mL) was added TFA (1 mL). The reaction mixture was stirred at r.t for 0.5 h. LCMS showed the reaction was completed. The reaction mixture removed under the reduced pressure. The residue was diluted with dichloromethane (100 mL). The solution was basified with NaHCO₃. The organic layer was washed with brine, dried over sodium sulfate, filtered, and concentrated to dryness under reduced pressure to give compound 8 (60 mg, Yield: 92%) as a white solid. MS m/z (ESI): 654.3 [M+H]⁺.

To a solution of compound 8 (60 mg, 0.92 mmol) and DIEA (356 mg, 2.76 mmol) in 1 mL of DMF, was added compound 9 (30 mg, 1.84 mmol). The reaction mixture was then stirred at r.t for 0.5 h. LCMS showed the reaction was completed. The crude was purified by prep-HPLC to give 261 (9.9 mg, Yield: 14.4%). MS: 749.3 [M+H]⁺.

¹H NMR (400 MHz, DMSO-d₆) δ 9.12 (s, 1H), 8.23 (s, 1H), 8.12 (d, J=9.2 Hz, 3H), 7.20 (dd, J=31.6, 7.2 Hz, 2H), 5.27 (d, J=53.6 Hz, 1H), 4.65 (d, J=11.2 Hz, 1H), 4.23-3.96 (m, 4H), 3.91-3.78 (m, 3H), 3.09 (d, J=9.2 Hz, 2H), 3.01 (s, 1H), 2.85-2.78 (m, 1H), 2.21-1.73 (m, 12H).

### Example R: Synthesis of (4R)-4-(4-(1-(1H-1,2,4-triazole-1-carbonyl)-1,6-diazaspiro[3.4]octan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (221)

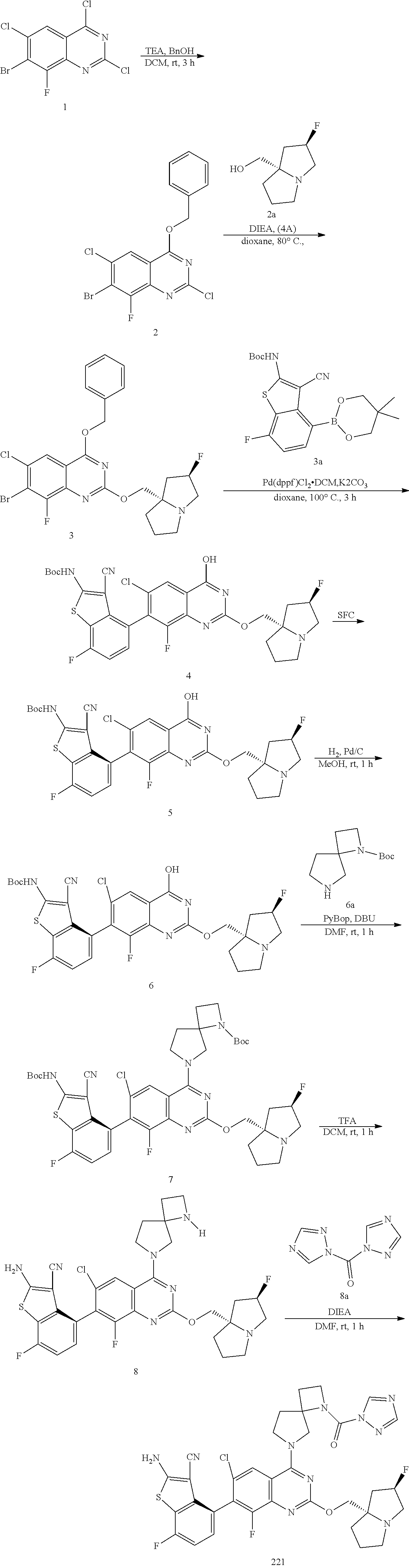

To a solution of compound 1 (5 g, 15.1 mmol) and TEA (4.59 g, 45.40 mmol) in DCM (50 mL) was added BnOH (1.96 g, 18.2 mmol). The mixture was stirred room temperature for 3 h. The mixture was poured into water (100 mL), and the solution was extracted with DCM (100 mL×3). The organic layer was washed with brine (50 mL), dried over anhydrous sodium sulfate, filtered and the filtrate was concentrated to dryness under reduced pressure, which was purified by silica gel column (PE/EA=10:1) to give compound 2 (4 g, Yield: 65.7%) as a yellow solid. MS m/z (ESI): 401.0 [M+H]⁺.

A mixture of compound 2 (5 g, 12.44 mmol), compound 2a (5.94 g, 37.31 mmol), molecular sieve and (4A) (500 mg), DIEA (4.82 g, 37.31 mmol) in DMSO (30 mL) was stirred at 80° C. for 16 h under N₂. The mixture was filtered and filtrate was extracted with EA (100 mL), washed with brine (50 mL×3), dried over anhydrous sodium sulfate, filtered and the filtrate was concentrated to dryness under reduced pressure. The residue was purified by silica gel column (PE/EA=1:1) to give compound 3 (2.3 g, Yield: 35%) as a white solid. MS m/z (ESI): 523.8 [M+H]⁺.

To a solution of compound 3 (2 g, 3.82 mmol), compound 3a (2.32 g, 5.73 mmol) and K₂CO₃ (3.16 g, 22.94 mmol) in dioxane (30 mL) was added Pd(dppf)Cl₂(624 mg, 0.76 mmol). The mixture was stirred 100° C. for 3 h under N₂. The mixture was poured into water (100 mL), and the solution was extracted with ethyl acetate (100 ml×3). The organic layer was washed with brine (50 mL), dried over anhydrous sodium sulfate, filtered and the filtrate was concentrated to dryness under reduced pressure. The residue was purified by silica gel column (MeOH:DCM=15:1) to yield compound 4 (1.4 g, Yield: 49.9%) as a yellow solid. MS m/z (ESI): 736.3 [M+H]⁺.

Compound 4 (4 g, 5.44 mmol) was separated by SFC to obtain Compound 5 (1.8 g, 45%) as yellow solid.

To a solution of compound 5 (1.8 g, 2.44 mmol) in MeOH (50 mL) was added Pd/C (180 mg). The mixture was stirred 25° C. for 0.5 h under H₂. The mixture was filtered and the filtrate was concentrated to dryness under reduced pressure to get compound 6 (1.4 g, Yield: 88.6%) as a grey solid. MS m/z (ESI): 646.4 [M+H]⁺.

To a solution of compound 6 (100 mg, 0.155 mmol), compound 6a (43 mg, 0.202 mmol) and PyBop (121 mg, 0.233 mmol) in DMF (2 mL) was added DBU (35 mg, 0.232 mmol). The mixture was stirred at room temperature for 1 h. The mixture was poured into water (50 mL), and the solution was extracted with ethyl acetate (50 ml×3). The organic layer was washed with brine (50 mL), dried over anhydrous sodium sulfate, filtered and the filtrate was concentrated to dryness under reduced pressure. The residue was purified by prep-TLC (MeOH:DCM=15:1) to get compound 7 (100 mg, Yield: 65.0%) as a yellow solid. MS m/z (ESI): 840.7 [M+H]⁺.

To a solution of compound 7 (100 mg, 0.12 mmol) in DCM (2 mL) was added TFA (1 mL). The reaction mixture was stirred at 20° C. for 1 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 8 (65 mg, 85.35%) as a yellow solid. MS m/z (ESI): 640.1 [M+H]⁺.

To a solution of compound 8 (80 mg, 0.125 mmol) and DIEA (48 mg, 0.376 mmol) in DMF (1 mL) was added compound 8a (51 mg, 0.313 mmol) at 0° C. under N₂. The mixture was stirred at room temperature for 1 h. The mixture was concentrated and purified by prep-HPLC (FA) to give 221 (24.11 mg, Yield: 26.24%) as a white solid.

¹H NMR (400 MHz, DMSO-d₆) δ 9.15 (d, J=2.8 Hz, 1H), 8.25 (d, J=7.2 Hz, 1H), 8.19 (s, 1H), 8.14 (s, 1H), 8.11 (s, 2H), 7.26-7.21 (m, 1H), 7.15 (t, J=8.8 Hz, 1H), 5.27 (d, J=53.6 Hz, 1H), 4.77-4.48 (m, 3H), 4.24 (s, 2H), 4.14-4.03 (m, 2H), 4.00-3.97 (m, 1H), 3.08 (d, J=9.6 Hz, 2H), 3.01 (s, 1H), 2.82 (d, J=6.0 Hz, 1H), 2.68-2.56 (m, 2H), 2.47-2.30 (m, 2H), 2.19-1.97 (m, 3H), 1.89-1.72 (m, 3H). MS m/z (ESI): 735.3 [M+H]⁺.

### Example S: Synthesis of 2,5-dioxopyrrolidin-1-yl 6-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-1,6-diazaspiro[3.4]octane-1-carboxylate 243

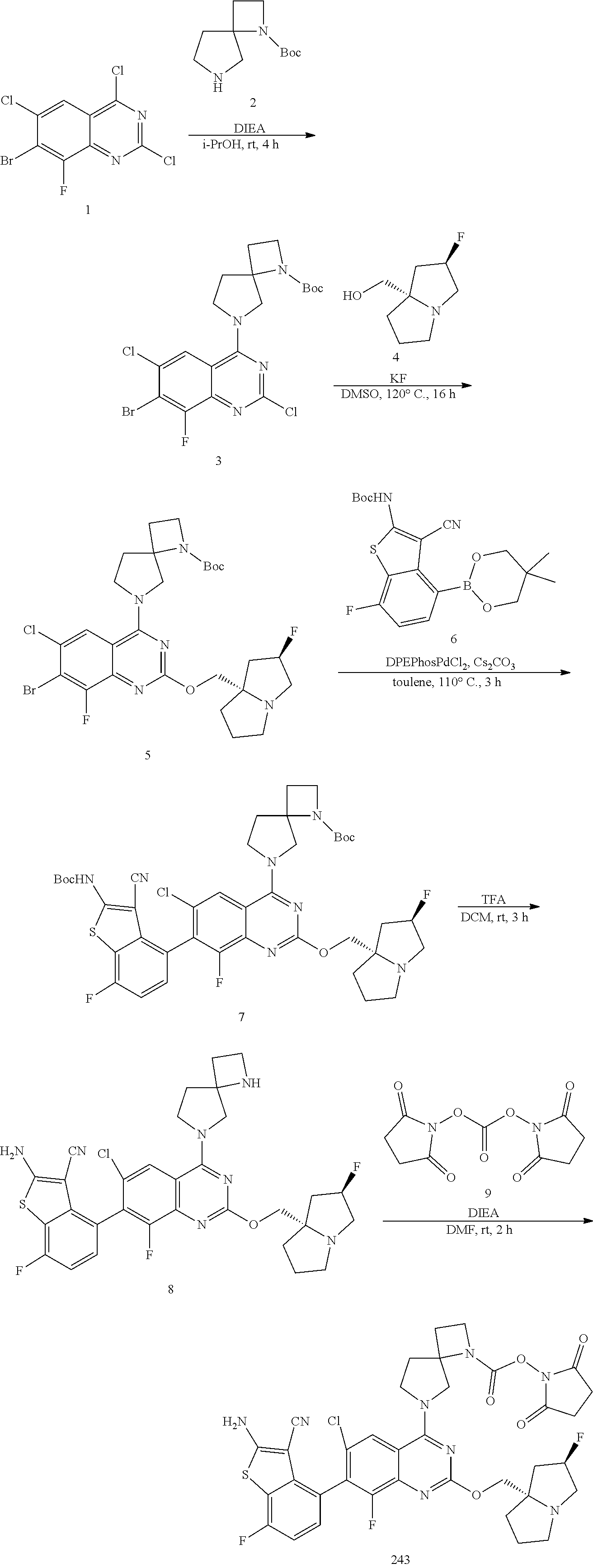

To a solution of compound 1 (1.5 g, 4.54 mmol) and compound 2 (964 mg, 4.54 mmol) in i-PrOH (15 mL) was added DIEA (1.76 g 13.62 mmol). The reaction mixture was stirred at 25° C. for 3 h. The mixture was filtered and the solid was diluted with PE (200 mL). The organic solution was concentrated to dryness under reduced pressure to give compound 3 (2 g, Yield: 87%) as a yellow solid. MS m/z (ESI): 505.1 [M+H]⁺.

To a solution of compound 3 (1 g, 1.98 mmol) and compound 4 (1.57 g, 9.88 mmol) in DMSO (15 mL) was added KF (574 mg, 9.88 mmol). The reaction mixture was stirred at 120° C. for 16 h. The mixture was poured into ice water (100 mL), and the solution was extracted with ethyl acetate (200 mL×3). The organic layer was washed with brine (200 mL), dried over anhydrous sodium sulfate, filtered and the filtrate was concentrated to dryness under reduced pressure. The residue was purified by silica gel column (PE/EA=1/1) to give compound 5 (550 mg, Yield: 44%) as a white solid. MS m/z (ESI): 628.2 [M+H]⁺.

To a solution of compound 5 (450 mg, 0.72 mmol), compound 2 (434 mg, 1.07 mmol), and Cs₂CO₃ (103 mg, 2.15 mmol) in toluene (10 mL) was added DPEPhosCl₂ (92 mg, 0.14 mmol). The mixture was stirred at 110° C. under nitrogen for 3 hours. The mixture was poured into water (200 mL), and the solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 7 (350 mg, Yield: 58%) as a white solid. MS m/z (ESI): 839.9 [M+H]⁺.

To a solution of compound 7 (350 mg, 0.42 mmol) in DCM (1 mL) was added TFA (1 mL). The reaction mixture was stirred at 25° C. for 1 h. The solvent was removed under reduced pressure. The reaction mixture was diluted with a.q. NaHCO₃ (150 mL) and DCM (50 mL). The organic layer was washed with brine, dried over sodium sulfate, filtered, and concentrated to afford compound 8 (300 mg, 82%) as a yellow solid. MS m/z (ESI): 640.6 [M+H]⁺.

To a solution of compound 8 (40 mg, 0.063 mmol) and DIEA (24 mg, 0.188 mmol) in 2 mL of DMF, was added compound 9 (24 mg, 0.094 mmol). The reaction mixture was then stirred at 25° C. for 1 h. The crude was purified by prep-HPLC to give 243 (9.63 mg, Yield: 20%). MS m/z (ESI): 781.3 [M+H]⁺.

¹H NMR (400 MHz, DMSO-d₆) δ 8.12 (d, J=11.8 Hz, 3H), 7.32-7.18 (m, 1H), 7.15 (t, J=8.8 Hz, 1H), 5.30 (d, J=54.4 Hz, 11H), 4.50-3.75 (m, 8H), 3.27-2.59 (m, 101H), 2.48-1.73 (m, 811).

### Example T: Synthesis of 4-(4-(1-(1H-1,2,4-triazole-1-carbonyl)-1,7-diazaspiro[4.5]decan-7-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile 208

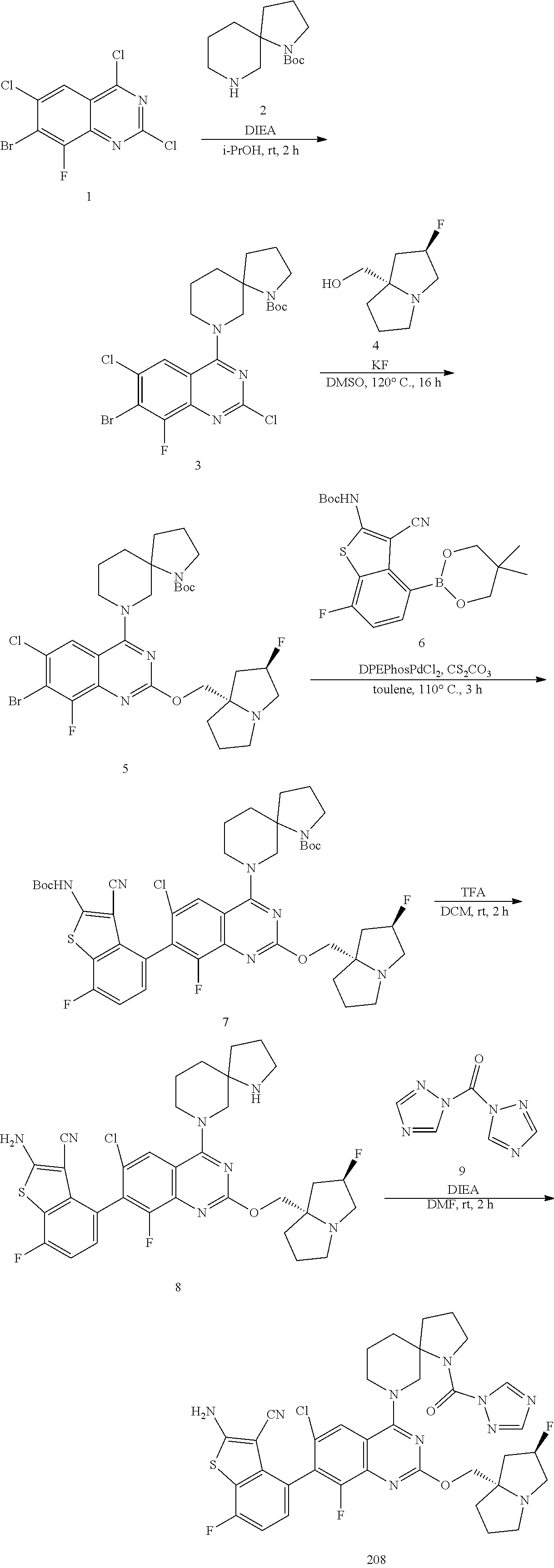

To a solution of compound 1 (1.20 g, 3.63 mmol) and compound 2 (872 mg, 3.63 mmol) in i-PrOH (50 mL) was added DIEA (1.41 g 10.89 mmol). The reaction mixture was stirred at 25° C. for 2 h. The mixture was poured into ice water (100 mL). The mixture was filtered and the solid was diluted with MeOH (100 mL). The organic solution was concentrated to dryness under reduced pressure to give compound 3 (1.75 g, Yield: 90.2%) as a white solid. MS m/z (ESI): 532.9 [M+H]⁺.

To a solution of compound 3 (700 mg, 1.31 mmol) and compound 4 (626 mg, 3.93 mmol) in DMSO (5 mL) was added KF (228 mg, 3.93 mmol). The reaction mixture was stirred at 120° C. for 16 h. The mixture was poured into water (50 mL), and the solution was extracted with ethyl acetate (50 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 5 (430 mg, Yield: 49.9%) as a yellow solid. MS m/z (ESI): 656.4 [M+H]⁺. To a solution of compound 5 (400 mg, 0.61 mmol), compound 6 (372 mg, 0.92 mmol), and Cs₂CO₃ (596 mg, 1.83 mmol) in toluene (5 mL) was added DPEPhosPdCl₂ (86 mg, 0.12 mmol). The mixture was stirred at 110° C. under nitrogen for 3 hours. The mixture was poured into water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 7 (180 mg, Yield: 34.1%) as a yellow solid. MS m/z (ESI): 868.2 [M+H]⁺.

To a solution of compound 7 (160 mg, 0.18 mmol) in DCM (10 mL) was added TFA (10 mL). The reaction mixture was stirred at 20° C. for 2 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 8 (100 mg, 81.30%) as a yellow solid. MS m/z (ESI): 668.4 [M+H]⁺.

To a solution of compound 8 (50 mg, 0.07 mmol) in DMF (3 mL) was added compound 9 (34 mg, 0.21 mmol) and DIEA (27 mg, 0.21 mmol). The mixture was stirred at 25° C. for 2 h. The residue was purified by Prep-HPLC (FA) to give compound 208 (2.19 mg, yield: 3.84%) as a white solid. MS m/z (ESI): 763.2 [M+H]⁺.

¹H NMR (400 MHz, DMSO-d₆) δ 9.08 (s, 1H), 8.20 (s, 1H), 8.10 (s, 2H), 7.89 (s, 1H), 7.24 (d, J=4.4 Hz, 1H), 7.19-7.12 (m, 11H), 5.27 (d, J=55.2 Hz, 1 Hz), 4.40-4.25 (m, 2H), 4.20-4.00 (m, 2H), 3.85-3.73 (m, 2H), 3.12-3.05 (m, 4H), 3.03-2.98 (m, 2H), 2.87-2.80 (m, 2H), 2.33 (s, 1H), 2.06-1.98 (m, 3H), 1.93-1.87 (m, 2H), 1.83-1.73 (m, 611).

### Example U: (R)-2-amino-4-(6-chloro-8-fluoro-4-((R)-1-(3-fluoro-1H-1,2,4-triazole-1-carbonyl)-1,6-diazaspiro[3.4]octan-6-yl)-2-(((2R,7aS)-2-fluorotetrahydro-H-pyrrolizin-7a(5H)-yl)methoxy)quinazolin-7-yl)-7-fluorobenzo[b]thiophene-3-carbonitrile (281)

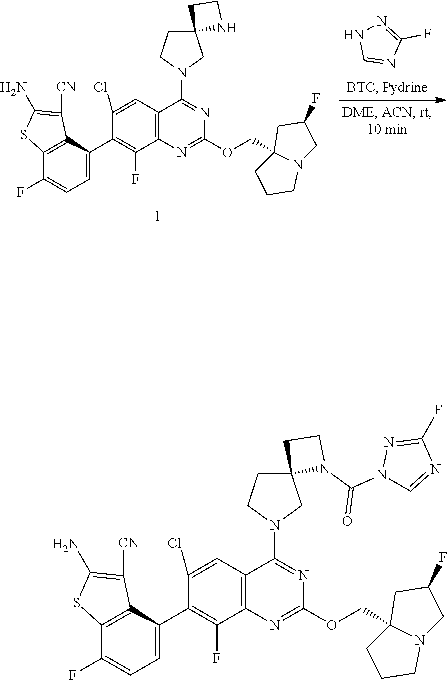

To a solution of compound 1a (162 mg, 1.86 mmol) and pyridine (0.6 mL, 7.5 mmol) in MeCN (4.5 mL) was added BTC (496 mg, 1.67 mmol) at 0° C. under N₂ atmosphere. The reaction mixture was then stirred at 0° C. for 2 h. To a solution of compound 1 (90 mg, 0.14 mmol) in DME (1.5 mL) was added pyridine (0.6 mL, 7.5 mmol) and above reaction mixture (0.5 mL) at 30° C. under N₂ atmosphere. LCMS showed the reaction was completed. The mixture was poured into water (15 mL), and the solution was extracted with ethyl acetate (15 mL×3). The organic layer was washed with brine (15 mL), dried over anhydrous sodium sulfate, filtered and the filtrate was concentrated to dryness under reduced pressure. The crude was purified by prep-HPLC (FA) to give Yield: GD-XL1027 (50 mg, 47.2%) as a white solid. MS m/z (ESI): 753.5 [M+H]⁺.

1H NMR (400 MHz, DMSO) δ 9.12 (s, 1H), 8.13-8.12 (m, 3H), 7.24-7.23 (m, 1H), 7.15-7.14 (m, 1H), 5.33 (d, J=54.0, 1H), 4.67-4.66 (m, 1H), 4.51-4.45 (m, 2H), 4.25-3.95 (m, 5H), 3.15-2.96 (m, 3H), 2.90-2.75 (m, 2H), 2.61-2.52 (m, 1H), 2.48-2.30 (m, 2H), 2.20-1.90 (m, 3H), 1.85-1.70 (m, 3H).

### Example V: Synthesis of 2-amino-4-(6-chloro-4-(1-((1R,2R)-2-cyanocyclopropane-1-carbonyl)-1,6-diazaspiro[3.5]nonan-6-yl)-8-fluoro-2-((S)-1-((S)-1-methylpyrrolidin-2-yl)ethoxy)quinazolin-7-yl)-7-fluorobenzo[b]thiophene-3-carbonitrile (235)

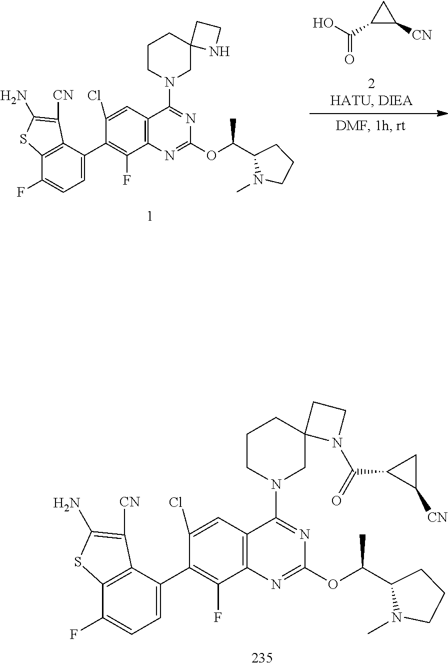 [SMILES: C[C@H](Oc1nc(N2CCCC3(CCN3)C2)c2cc(Cl)c(-c3ccc(F)c4sc(N)c(C#N)c34)c(F)c2n1)[C@@H]1CCCN1C ; N#C[C@@H]1C[C@H]1C(=O)O ; C[C@H](Oc1nc(N2CCCC3(CCN3C(=O)[C@@H]3C[C@H]3C#N)C2)c2cc(Cl)c(-c3ccc(F)c4sc(N)c(C#N)c34)c(F)c2n1)[C@@H]1CCCN1C]

To a solution of compound 2 (71 mg, 0.64 mmol) and DIEA (165 mg, 1.28 mmol) in DMF (10 mL) was added HATU (243 mg, 0.64 mmol), the mixture was stirred at 25° C. for 1 h. To a solution of compound 1 (40 mg, 0.064 mmol) in DMF (1 mL) was added above reaction mixture (1 mL) at 25° C. The mixture was stirred at 25° C. for 1 h. The solution was purified by prep-HPLC to give 235 (4.43 mg, Yield: 9.62%) as a white solid.

¹H NMR (400 MHz, DMSO-d₆) δ 8.11 (s, 2H), 7.82 (d, J=14.4 Hz, 1H), 7.28 (s, 1H), 7.16 (t, J=8.4 Hz, 1H), 5.27 (s, 1H), 4.51-4.07 (m, 3H), 3.86-3.65 (m, 11H), 3.22-3.02 (m, 11H), 2.95 (t, J=6.4 Hz, 11H), 2.69-2.60 (m, 11H), 2.40-2.24 (m, 5H), 2.21-1.93 (m, 5H), 1.87-1.59 (m, 6H), 1.50-1.20 (m, 6H). MS m/z (ESI): 717.3 [M+H]⁺.

### Example W: Synthesis of 4-(4-(1-(1H-1,2,4-triazole-1-carbonyl)-1,6-diazaspiro[3.5]nonan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile 275

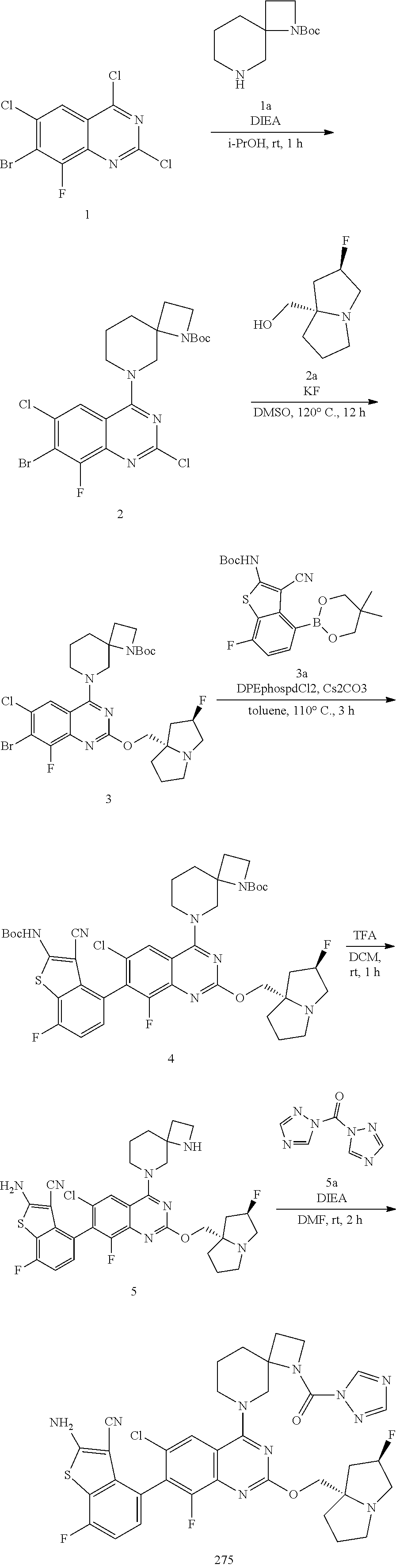

To a solution of compound 1 (360 mg, 1.09 mmol) and 1a (247 mg, 1.09 mmol) in i-PrOH (15 mL) was added DIEA (423 mg, 3.27 mmol). The reaction mixture stirred at rt for 1 h. The reaction solution was filtered with petroleum ether. The filter cake is concentrated to dry state under reduced pressure to give compound 2 (500 mg, Yield: 88.6%) as yellow solid. MS m/z (ESI): 521.0 [M+H]⁺.

To a solution of compound 2 (500 mg, 0.96 mmol) in DMSO (5 mL), was added 2a (458 mg, 2.88 mmol) and KF (167 mg, 0.96 mmol). The mixture was stirred at 120° C. for 12 h. The solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 3 (340 mg, Yield: 55.2%) as a yellow solid. MS m/z (ESI): 644.1 [M+H]⁺.

To a solution of compound 3 (340 mg, 0.53 mmol), 3a (321 mg, 0.795 mmol), and Cs₂CO₃ (518 mg, 1.59 mmol) in toluene (15 mL) was added DPEPhosPdCl₂ (76 mg, 0.106 mmol). The mixture was stirred at 110° C. for 3 h. The solution was extracted with ethyl acetate (100 mL×3). The organic layer was washed with brine (100 mL), dried over anhydrous sodium sulfate, filtered and the filtrate was concentrated to dryness under reduced pressure. The residue was purified by reverse phase column (MeCN/H₂O(4% FA)=1/3) to give compound 4 (130 mg, Yield: 28.8%) as a yellow solid. MS m/z (ESI): 854.5 [M+H]⁺.

To a solution of compound 4 (130 mg, 0.083 mmol) in DCM (6 mL) was added TFA (3 mL). The reaction mixture was stirred at rt for 1 h. The solvent was removed under reduced pressure. The reaction mixture was diluted with a.q. NaHCO₃ (200 mL) and DCM/MeOH=10/1(300 mL). The organic layer was washed with brine, dried over sodium sulfate, filtered, and concentrated to give compound 5 (80 mg, Yield: 80.4%) as a yellow solid. MS m/z (ESI): 654.1 [M+H]⁺.

To a solution of compound 5 (80 mg, 0.122 mmol) and DIEA (47 mg, 0.366 mmol) in 5 mL of DMF, was added 5a (60 mg, 0.366 mmol). The reaction mixture was then stirred at rt for 2 h. LCMS showed the reaction was completed. The crude was purified by Prep-HPLC to give 275 (13.81 mg, Yield: 15.07%). MS m/z (ESI): 749.3 [M+H]⁺.

¹H NMR (400 MHz, DMSO-d₆) δ 9.15 (d, J=1.6 Hz, 1H), 8.25 (s, 2H), 8.12 (s, 2H), 7.87 (d, J=23.6 Hz, 1H), 7.27-7.24 (m, 11H), 7.21-7.09 (m, 11H), 5.27 (d, J=54.0 Hz, 11H), 4.61-4.53 (m, 3H), 4.29-4.18 (m, 11H), 4.14-4.08 (m, 11H), 4.05-3.93 (m, 2H), 3.19 (s, 1H), 3.08 (d, J=10.4 Hz, 2H), 3.01 (s, 1H), 2.82 (d, J=6.0 Hz, 1H), 2.25-1.75 (m, 1211).

### Example X: Synthesis of 2-amino-4-(6-chloro-4-(1-(2,2-difluorocyclopropane-1-carbonyl)-3,3-difluoro-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (228)

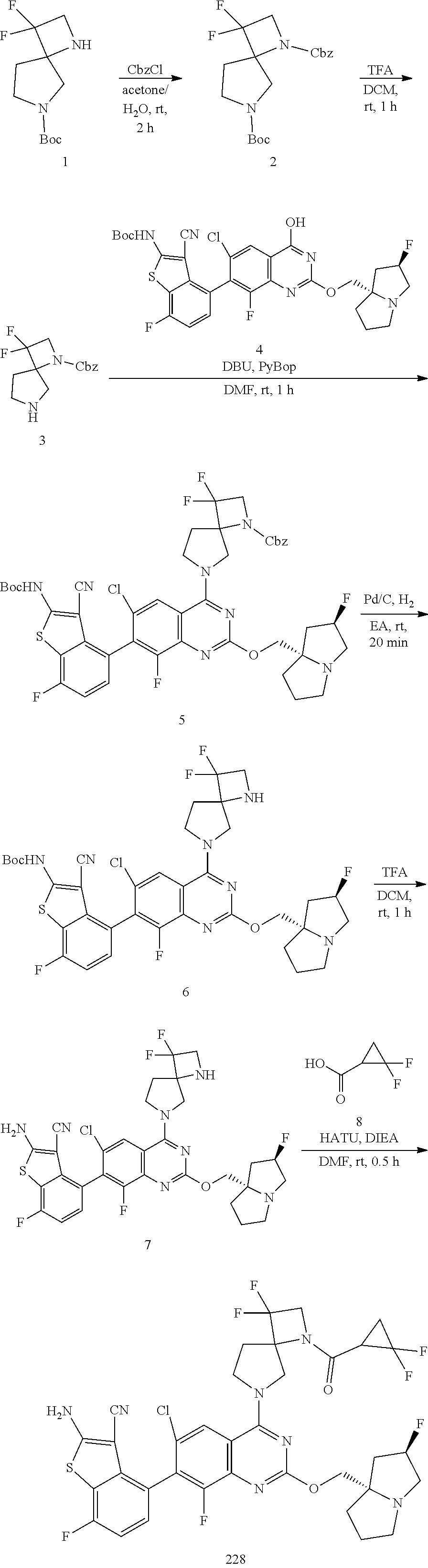

To a solution of compound 1 (100 mg, 0.40 mmol) and NaHCO₃(68 mg, 0.80 mmol) in acetone (2 mL) was added H₂O (1 mL). The reaction mixture was added CbzCl (137 mg, 0.80 mmol) stirred at 25° C. for 1 h. The solution was extracted with ethyl acetate (50 mL×3). The organic layer was washed with brine (50 mL), dried over anhydrous sodium sulfate, filtered and the filtrate was concentrated to dryness under reduced pressure. The residue was purified by silica gel column (PE/EA=6/1) to give compound 2 (80 mg, Yield: 52%) as colorless oily liquid.

To a solution of compound 2 (80 mg, 0.21 mmol) in DCM (1 mL) was added TFA (1 mL). The reaction mixture was stirred at 20° C. for 1 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 3 (50 mg, 85%) as colorless oily liquid.

To a solution of compound 3 (119 mg, 0.42 mmol), compound 4 (210 mg, 0.32 mmol), and PyBop (253 mg, 0.49 mmol) in DMF (3 mL) was added DBU (61 mg, 0.49 mmol). The mixture was stirred at 25° C. for 1 h. The solution was extracted with ethyl acetate (80 mL×3). The organic layer was washed with brine (100 mL), dried over anhydrous sodium sulfate, filtered and the filtrate was concentrated to dryness under reduced pressure. The residue was purified by silica gel column (DCM/MeOH=15/1) to give compound 5 (170 mg, Yield: 58%) as a white solid. MS m/z (ESI): 910.2 [M+H]⁺.

To a solution of compound 5 (120 mg, 0.13 mmol) in ethyl acetate (3 ml) was added Pd/C (60 mg). The reaction mixture was stirred at rt under H₂ atmosphere for 20 min. The reaction mixture was diluted with ethyl acetate (30 ml). The organic layer was filtered, and concentrated to afford compound 6 (90 mg, 89%) as a yellow solid. MS m/z (ESI): 776.2 [M+H]⁺.

To a solution of compound 6 (90 mg, 0.17 mmol) in DCM (2 mL) was added TFA (2 mL). The reaction mixture was stirred at 20° C. for 30 min. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 7 (70 mg, 89%) as colorless oily liquid. MS m/z (ESI): 676.3 [M+H]⁺.

To a solution of compound 8 (45 mg, 0.37 mmol) and DIEA (96 mg, 0.74 mmol) in 5 mL of DMF was added HATU (281 mg, 0.74 mmol) at 0° C. under N₂ atmosphere. The reaction mixture was then stirred at 25° C. for 30 min. To a solution of compound 7 (30 mg, 0.044 mmol) in DMF (0.6 mL) was added above reaction mixture (0.6 mL). The mixture was stirred at 25° C. for 5 min. The mixture was poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by Prep-HPLC (FA) to give 228 (2.10 mg, Yield: 10%) as a white solid.

¹H NMR (400 MHz, DMSO-d₆) δ 8.13 (d, J=16.2 Hz, 3H), 7.30-7.21 (m, 1H), 7.15 (t, J=8.8 Hz, 1H), 5.28 (d, J=54.4 Hz, 1H), 4.88-4.02 (m, 7H), 3.20-2.66 (m, 6H), 2.15-1.73 (m, 8H), 1.53-0.92 (m, 211).

### Example Y: Synthesis of (4R)-4-(4-(1-(1H-1,2,4-triazole-1-carbonyl)-6-oxa-1,9-diazaspiro[3.6]decan-9-yl)-6-chloro-8-fluoro-2-carbonitrile (291)

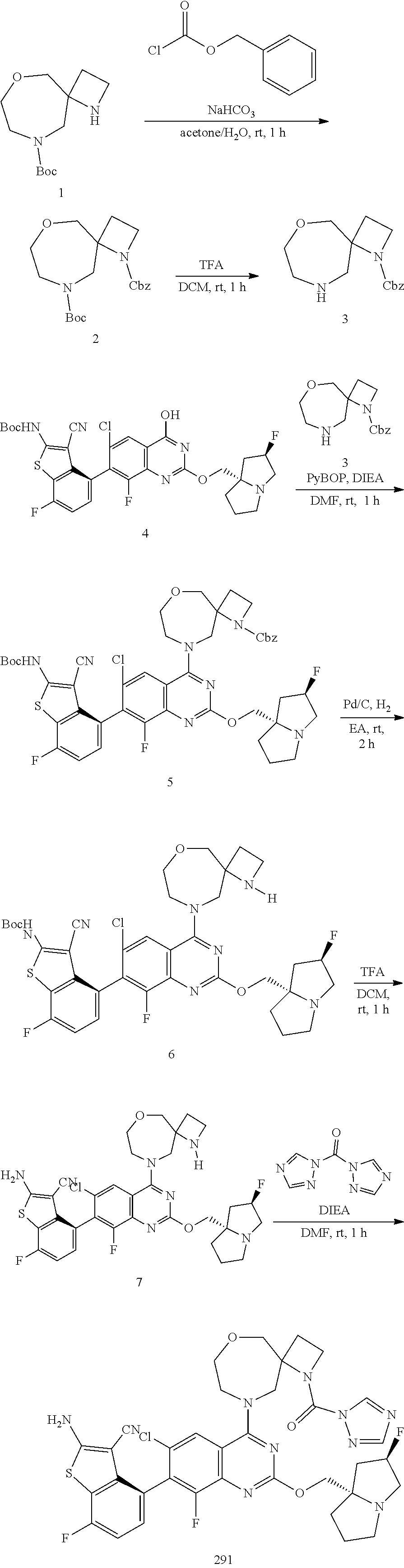

To a solution of compound 1 (150 mg, 0.619 mmol) and NaHCO₃(158 mg, 0.929 mmol) in Acetone/H₂O (10 mL) was added compound 1A (104 mg, 1.24 mmol) at 0° C. The mixture was stirred at room temperature for 1h. The mixture was poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by SGC (PE/EA=10:1) to give compound 2 (190 mg, Yield: 81.5%) as a yellow solid.

To a solution of compound 2 (190 mg, 0.504 mmol) in DCM (1 mL) was added TFA (1 mL). The reaction mixture was stirred at rt for 1 h. LCMS showed the reaction was completed. The reaction mixture concentrated to dryness under the reduced pressure. The residue was diluted with dichloromethane (20 mL). The solution was neutralized with NaHCO₃. The organic layer was washed with brine, dried over sodium sulfate, filtered, and concentrated to dryness under reduced pressure to give compound 3 (80 mg, crude) as a yellow solid. MS m/z (ESI): 277.1 [M+H]

To a solution of compound 4 (100 mg, 0.155 mmol) and PyBop (121 mg, 0.233 mmol) in DMF (2 mL) was added compound 3 (56 mg, 0.201 mmol) and DIEA (40 mg, 0.31 mmol). The mixture was stirred at room temperature for 1h. The mixture was poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by prep-TLC (DCM/MeOH=15:1) to give compound 5 (120 mg, Yield: 85.7%) as a yellow solid. MS m/z (ESI): 904.2 [M+H]⁺

To a solution of compound 5 (120 mg, 0.133 mmol) in EA (5 mL) was added Pd/C (20 mg). The reaction mixture was stirred at rt under H₂ for 1 h. LCMS showed the reaction was completed. The reaction mixture was diluted with EA, filtered through diatomaceous earth, and concentrated to dryness under reduced pressure. The residue was purified by prep-TLC (DCM/MeOH=8:1) to give compound 6 (30 mg, 29.4%) as a yellow solid. MS m/z (ESI): 770.2 [M+H]⁺.

To a solution of compound 6 (30 mg, 0.039 mmol) in DCM (1 mL) was added TFA (1 mL). The reaction mixture was stirred at rt for 1 h. LCMS showed the reaction was completed. The reaction mixture was concentrated under the reduced pressure. The residue was diluted with dichloromethane (20 mL). The solution was neutralized with NaHCO₃. The organic layer was washed with brine, dried over sodium sulfate, filtered, and concentrated to dryness under reduced pressure to give compound 7 (30 mg, crude) as a yellow solid. MS m/z (ESI): 670.2 [M+H]⁺.

To a solution of compound 7 (30 mg, 0.045 mmol) and DIEA (17 mg, 0.135 mmol) in 2 mL DMF, was added 7a (22 mg, 0.134 mmol). The reaction mixture was then stirred at rt for 1 h. LCMS showed the reaction was completed. The crude was purified by Prep-HPLC to give 291 (2.8 mg, Yield: 8.17%) as a white solid. MS m/z (ESI): 765.2 [M+H]⁺.

¹H NMR (400 MHz, DMSO-d₆) δ 9.08 (d, J=2.8 Hz, 1H), 8.35 (s, 1H), 8.25 (d, J=2.4 Hz, 1H), 8.12 (s, 2H), 7.98 (d, J=31.2 Hz, 1H), 7.28-7.12 (m, 2H), 5.25 (d, J=54.8 Hz, 1H), 4.89-4.72 (m, 1H), 4.65-4.41 (m, 3H), 4.39-4.27 (m, 1H), 4.17-3.97 (m, 6H), 3.92-3.80 (m, 1H), 3.11-3.04 (m, 2H), 3.00-2.96 (m, 1H), 2.85-2.76 (m, 1H), 2.40-2.32 (m, 2H), 2.13-2.05 (m, 1H), 2.04-1.93 (m, 2H), 1.84-1.70 (m, 311).

### Example Z: Synthesis of 4-((7R)-4-(1-(1H-1,2,4-triazole-1-carbonyl)-1,6-diazaspiro[3.6]decan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (316)

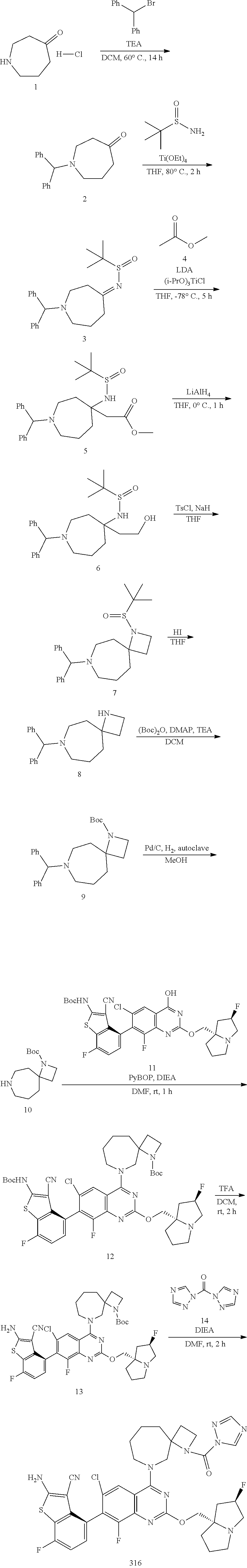

To a solution of 1 (1 g, 6.7 mmol) and TEA (1.35 mg, 13.4 mmol) in dry THF (10 mL) was added 1a (1.65 mg, 6.7 mmol) at 0° C. The mixture was stirred at 25° C. for 12 h. The reaction mixture was diluted with water (100 mL) and extracted with EA (100 mL×3). The organic layer was washed with brine (20 mL), dried over anhydrous sodium sulfate, filtered and the filtrate was concentrated to dryness under reduced pressure. The residue was purified by column on silica gel to give compound 2 as colorless oil. (1 g, Yield: 53%).

A mixture of compound 2 (1 g, 3.582 mmol), compound 2a (650 mg, 5.37 mmol) and Ti(OEt)₄ (1.6 g, 7.1 mmol) in THF (10 ml) was stirred at 80° C. for 2 h. The mixture was added Na₂SO₄.10H₂O (100 mg) in ice bath, and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=9/20) to give compound 3 (670 mg, Yield: 48.9%) as a white solid.

N-butyllithium (2.5 M in hexanes, 9.4 mL, 23.55 mmol) was added dropwise at 0° C. to a solution of Diisopropylamine (1.99 g, 19.63 mmol) in dry THF (30 mL) and the solution was stirred at 0° C. for 0.5 h. The reaction mixture was then cooled to −78° C. followed by dropwise addition of a solution of 4 (1.16 g, 15.7 mmol). The resulting reaction mixture was stirred at −78° C. for 1.5 h. After this time, a solution of (i-PrO)₃TiCl (24 mL, 23.56 mmol) was added dropwise and the reaction stirred for 1 h. A solution of sulfonamide 3 (3 g, 7.85 mmol) in THF (10 mL) was then added dropwise and the reaction stirred for an additional 1 h. The reaction mixture was diluted with NH₄Cl (aq) (50 mL) and extracted with EA (50 ml×3). The organic layer was washed with brine (20 mL), dried over anhydrous sodium sulfate, filtered and the filtrate was concentrated to dryness under reduced pressure. The residue was purified by column on silica gel to give compound 5 as colorless oil. (1.9 g, Yield: 53%). MS m/z (ESI): 457.3[M+H]⁺.

To a solution of 5 (2.3 g, 5.04 mmol) in dry THF (30 mL) was added LiAlH₄ (7.6 mL, 7.6 mmol) at 0° C. The mixture was stirred at 25° C. for 1 h. Water (0.3 mL), 2 mmol/L NaOH solution (0.3 mL) and water (0.6 mL) were added. The reaction solution was filtered, and the solution was extracted with ethyl acetate (100 mL×3). Dried over anhydrous sodium sulfate, filtered and the filtrate was concentrated to dryness under reduced pressure The residue was purified by reversed phase column to give compound 6 (1.6 g, Yield: 81%) as colorless oil. MS m/z (ESI): 429.2[M+H]⁺.

To a solution of 6 (800 mg, 1.869 mmol) and TsCl (535 mg, 2.804 mmol) in dry THF (10 mL) was added NaH (300 mg, 7.48 mmol) at 0° C. The mixture was stirred at 25° C. for 2 h. The reaction mixture was diluted with NH₄Cl (aq) (20 mL) and extracted with EA (20 mL×3). The organic layer was washed with brine (20 mL), dried over anhydrous sodium sulfate, filtered and the filtrate was concentrated to dryness under reduced pressure. The residue was purified by column on silica gel to give compound 7 as colorless oil. (510 mg, Yield: 65%). MS m/z (ESI): 411.2 (M+H)⁺.

To a solution of 7 (300 mg, 0.732 mmol) in THF (2 mL) was added HI (937 mg, 7.32 mmol) at 0° C. The mixture was stirred at 25° C. for 2 h. The solvent was removed under reduced pressure. The reaction mixture was diluted with NaHCO₃(aq) (20 mL) and DCM (50 mL). The organic layer was washed with brine, dried over sodium sulfate, filtered, and concentrated to afford compound 8 (400 mg, crude) as a colorless oil. MS m/z (ESI): 307.2 [M+H]⁺.

To a solution of 8 (400 mg, crude), TEA (303 mg, 3 mmol) and DMAP (24 mg, 0.2 mmol) in DCM (4 mL) was added (Boc)₂O (327 mg, 1.5 mmol) at 0° C. The mixture was stirred at 25° C. for 16 h. The reaction mixture was diluted with H₂O (10 mL) and extracted with EA (10 mL×3). The organic layer was washed with brine (20 mL), dried over anhydrous sodium sulfate, filtered and the filtrate was concentrated to dryness under reduced pressure. The residue was purified by column on silica gel to give compound 9 as colorless oil. (250 mg, Yield: 15%). MS m/z (ESI): 407.3

To a solution of compound 9 (70 mg, 0.172 mmol) dissolved in EA (15 mL) in autoclave was added Pd(OH)₂ (70 mg) stirred at 50° C. under 2 Mpa H₂ for 1.5 h. The TLC (PE:EA=6/1) show the reaction was completed. The reaction mixture was diluted with methanol, filtered through diatomaceous earth and concentrated in vacuo to give compound 10 (70 mg, crude) as a colorless oily liquid.

To a solution of compound 11 (70 mg, 0.109 mmol), compound 10 (39 mg, 0.164 mmol) and PyBop (85 mg, 0.164 mmol) in DMF (2 ml) was added DIEA (21 mg, 0.164 mmol). The reaction mixture was stirred at 25° C. for 1 h. The mixture was poured into ice water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by prep-TLC (DCM/MeOH=15/1) to give compound 12 (50 mg, Yield: 53%) as a yellow solid. MS m/z (ESI): 868.2 [M+H]

To a solution of compound 12 (50 mg, 0.058 mmol) in DCM (1 ml) was added TFA (1 ml). The reaction mixture was stirred at rt for 2 h. The solvent was removed under reduced pressure. The reaction mixture was diluted with a.q. NaHCO₃ (20 mL) and DCM (50 mL). The organic layer was washed with brine (50 mL), dried over anhydrous sodium sulfate, filtered and the filtrate was concentrated to dryness under reduced pressure. The residue was compound 13 (30 mg, Yield: 77%) as a yellow solid. MS m/z (ESI): 668.3 [M+H].

To a solution of compound 13 (30 mg, 0.045 mmol) and DIEA (17 mg, 0.135 mmol) in 0.8 mL DMF, was added compound 14 (22 mg, 0.135 mmol). The reaction mixture was then stirred at rt for 1 h. LCMS showed the reaction was completed. The crude was purified by prep-HPLC to give 316 (10.66 mg, Yield: 31%). MS m/z (ESI): 763.2 [M+H].

1H NMR (400 MHz, DMSO-d₆) δ 9.06 (d, J=16.4 Hz, 1H), 8.24 (d, J=5.2 Hz, 1H), 8.12 (d, J=14.8 Hz, 2H), 7.97 (d, J=23.4 Hz, 1H), 7.31-7.20 (m, 1H), 7.15 (t, J=8.4 Hz, 1H), 5.26 (d, J=47.2 Hz, 1H), 4.86 (m, 1H), 4.60-3.73 (m, 7H), 3.09- 2.58 (m, 4H), 2.43-1.52 (m, 14H).

### Example AA: Synthesis of 4-(4-(1-(1H-1,2,4-triazole-1-carbonyl)-1,7-diazaspiro[4.6]undecan-7-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (317)

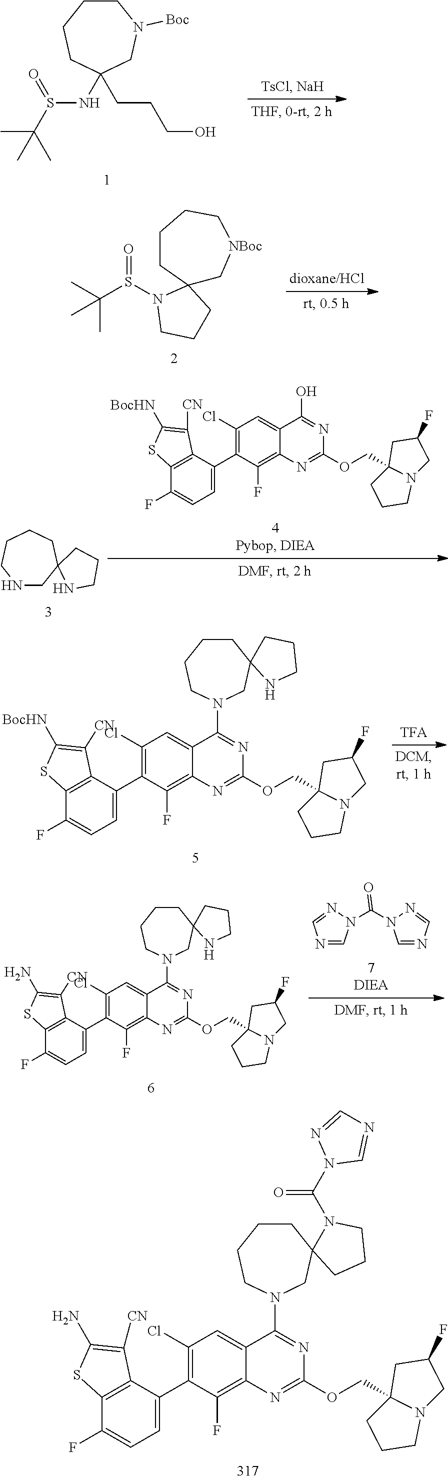

To a solution of compound 1 (1.4 g, 3.72 mmol) in dry THF (15 mL). At 0° C., add TsCl (1.06 g, 5.58 mmol) and NaH (595.2 mg, 14.88 mmol) to the reaction solution. The reaction was stirred at room temperature for 2 hr. The reaction solution was quenched with NH₄Cl solution. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=20/1) to give compound 2 (300 mg, Yield: 22.5%) as a white solid.

¹H NMR (400 MHz, DMSO-d₆) δ 4.02-3.67 (m, 2H), 3.64-3.50 (m, 1H), 3.11-2.99 (m, 1H), 2.95-2.82 (m, 2H), 2.10-1.99 (m, 1H), 1.89-1.71 (m, 5H), 1.59-1.51 (m, 2H), 1.42-1.38 (m, 9H), 1.34-1.22 (m, 2H), 1.14-1.10 (m, 9H).

To a solution of compound 3 (300 mg, 0.84 mmol) in dioxane/HCl (4 mL). The reaction mixture was stirred at rt for 0.5 h. The raw material disappears and new points are created by TLC. The reaction liquid compound 8 was obtained directly (125 mg, Yield: 96.9%) as a white solid.

To a solution of compound 4 (132.6 mg, 0.205 mmol), compound 3 (63.1 mg, 0.41 mmol), DIEA (52.9 mg, 0.41 mmol) and PyBop (213.2 mg, 0.41 mmol) in dry DMF (2 mL). The mixture was stirred at rt for 2 hr under N₂. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM:MeOH=20:1) to give compound 5 (60 mg, Yield: 37.5%) as a white solid. MS m/z (ESI): 782.5 [M+H]⁺.

To a solution of compound 7 (60 mg, 0.077 mmol) in DCM (1.5 ml) was added TFA (1.5 ml). The reaction mixture was stirred at rt for 30 min. The solvent was removed under reduced pressure. The reaction mixture was diluted with a.q. NaHCO₃ (20 mL) and DCM/MeOH=10/1(30 mL). The organic layer was washed with brine, dried over sodium sulfate, filtered, and concentrated to give compound 8 (50 mg, Yield: 96.2%) as a yellow solid. MS m/z (ESI): 682.5 [M+H]⁺.

To a solution of compound 6 (10 mg, 0.014 mmol) and DIEA (5.4 mg, 0.042 mmol) in DMF (1 mL) was added compound 7 (6.1 mg, 0.037 mmol) at rt under N₂. The mixture was stirred at room temperature for 1h. The mixture was concentrated and purified by prep-HPLC (FA) to give 317 (1.30 mg, Yield: 12.0%) as a white solid.

¹H NMR (400 MHz, DMSO-d₆) δ 9.10-9.00 (m, 1H), 8.25-8.05 (m, 3H), 8.00-7.85 (m, 1H), 7.28-7.14 (m, 2H), 5.25 (d, J=55.2 Hz, 1H), 4.17-3.95 (m, 4H), 3.83-3.77 (m, 2H), 3.10-3.03 (m, 4H), 2.80-2.75 (m, 2H), 2.18-2.15 (m, 1H), 2.04-1.90 (m, 7H), 1.85-1.70 (m, 811).

### Example AB: Synthesis of 2-amino-4-(6-chloro-4-(7-((difluoromethoxy)methyl)-1-(1H-1,2,4-triazole-1-carbonyl)-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (327)

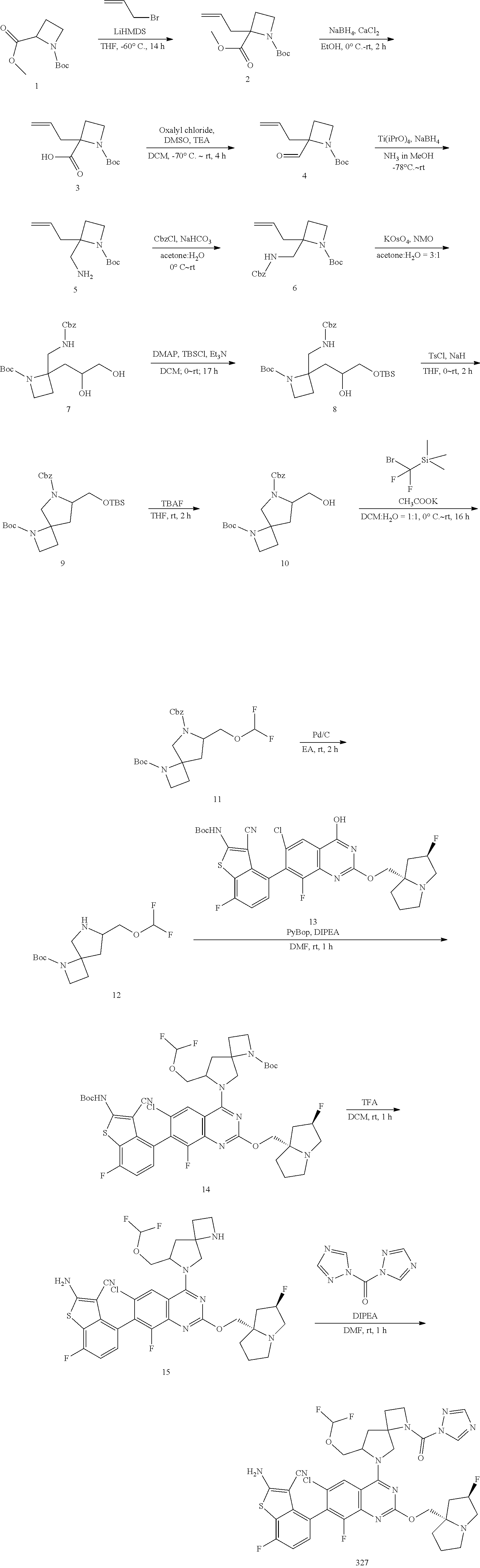

To a solution of compound 1 (20 g, 93.02 mmol) in THF (200 mL) was added LiHMDS (186 mL, 186.04 mmol). The mixture was stirred for 1h. And then was added 3-bromo-prop-1-ene (12.38 g, 102.32 mmol). The reaction mixture was stirred at rt for 14 h. The mixture was poured into a.q. NH₄Cl (50 mL) and extracted with ethyl acetate (50 mL×3). The organic layer was washed with brine (50 mL), dried over anhydrous sodium sulfate, filtered and the filtrate was concentrated to dryness under reduced pressure. The residue was purified by prep-TLC (PE/EA=10/1) to give compound 2 (14 g, Yield: 59%) as a yellow oil.

1H NMR (400 MHz, CDCl₃) δ 5.98-5.85 (m, 1H), 5.20 (m, 2H), 3.95 (m, 1H), 3.83-3.74 (s, 3H), 3.68 (m, 1H), 2.96-2.70 (m, 1H), 2.60 (m, 1H), 2.26 (m, 1H), 2.19-2.09 (m, 1H), 1.53-1.32 (m, 9H).

To a solution of compound 2 (14 g, 54.90 mmol) in EtOH (140 mL) was added CaCl₂ (12.19 g, 109.80 mmol) and NaBH₄ (8.34 g, 219.6 mmol) at 0° C. The reaction mixture was stirred at rt for 2 h. The solvent was quenched with Citric acid in water (1M/50 mL) and extracted with ethyl acetate (50 mL×3). The organic layer was washed with brine (50 mL), dried over anhydrous sodium sulfate, filtered and the filtrate was concentrated to dryness under reduced pressure. The residue was purified by prep-TLC (PE/EA=3/1) to give compound 3 (8 g, Yield: 64%) as a white solid.

1H NMR (400 MHz, CDCl₃) δ 5.86-5.85 (m, 1H), 5.24-5.13 (m, 2H), 3.81-3.80 (m, 1H), 3.57-3.56 (m, 1H), 3.29-3.28 (m, 1H), 2.65-2.64 (m, 2H), 2.20-1.56 (m, 3H), 1.45-0.44 (m, 9H).

To a mixture of Oxalyl chloride (2.22 g, 17.6 mmol) in DCM (16 mL) was added DMSO (2.75 g, 35.2 mmol) at −65° C. and stirred for 30 min. And then the mixture was added 3 (2 g, 8.8 mmol) in DCM (4 mL). The reaction was stirred for 1h at −65° C. After the reaction was added Et₃N (5.34 g, 52.8 mmol) and stirred for 20 min at −65° C. The mixture was stirred for 30 min at 0° C. The reaction mixture was quenched with water (15 mL) and stirred for 30 min at rt, dried over Na₂SO₄. The crude mixture was added THF:DCM=5:1 (50 mL). And then was stirred for 10 min and filtered. The filtrated was concentrated under vacuum and purified by prep-TLC (PE/EA=5/1) to give compound 4 (1.6 g, Yield: 73%) as a yellow oil. MS m/z (ESI): 170 [M+H-56].

1H NMR (400 MHz, DMSO) δ 9.69-9.68 (m, 1H), 5.89-5.87 (m, 1H), 5.18-5.17 (m, 2H), 3.85-3.42 (m, 2H), 2.64-2.63 (m, 1H), 2.50-2.40 (m, 1H), 2.33-2.04 (m, 2H), 1.45-1.27 (m, 9H).

To a mixture of 4 (9 g, 40 mmol) in NH₃ in MeOH (90 mL) was added Ti(iPrO)₄ (13.6 g, 48 mmol) at −65° C. and stirred for 16 h at rt. And then the reaction was added NaBH₄ (756 mg, 20 mmol). The mixture was stirred for 2 h at rt. After the reaction was filtrated and the filtrate was concentrated under vacuum to afford 5 (13 g, crude) as a yellow oil. MS m/z (ESI): 227 [M+H]⁺.

1H NMR (400 MHz, DMSO) δ 5.91-5.78 (m, 1H), 5.15-5.14 (m, 2H), 3.66-3.50 (m, 2H), 3.18-3.17 (m, 1H), 2.83-2.82 (m, 1H), 2.70-2.54 (m, 1H), 2.48-2.40 (m, 1H), 2.35-2.20 (m, 1H), 2.19-2.00 (m, 1H), 1.36-1.35 (m, 9H), 1.29-1.13 (m, 2H).

To a solution of 5 (9 g, 39.8 mmol) in Acetone (72 mL) and H₂O (18 mL) was added CbzCl (7.49 g, 43.78 mmol) and NaHCO₃ (13.38 g, 159.2 mmol) at 0° C. The mixture was stirred for 16h at rt. The reaction mixture was quenched with water (100 mL) and extracted with EA (3*50 mL). The combined organic was dried over Na₂SO₄. And then the mixture was filtered and the filtrate was concentrated to dryness under reduced pressure. The crude mixture was purified by flash chromatography to afford 6 (7 g, 49%) as a yellow oil. MS m/z (ESI): 305 [M+H-56].

1H NMR (400 MHz, DMSO) δ 7.39-7.32 (m, 5H), 5.98-5.74 (m, 1H), 5.14 (dt, J=16.9, 8.0 Hz, 4H), 4.75-4.74 (m, 1H), 3.69-3.37 (m, 2H), 3.32-3.14 (m, 1H), 2.71-2.51 (m, 1H), 2.49-2.42 (m, 1H), 2.41-2.01 (m, 2H), 1.97-1.77 (m, 1H), 1.42-1.27 (m, 9H).

To a solution of 6 (7.8 g, 21.67 mmol) in ACE (60 mL) and H₂O (20 mL) was added NMO (7.6 g, 65.01 mmol) and K₂OsO₄ (663 mg, 2.167 mmol) at rt. The mixture was stirred for 16 h at rt. After then the reaction mixture was quenched with Na₂SO₃ (80 mL). The aqueous layer was extracted with EA (2×50 mL). The combined organic extracts were dried over Na₂SO₄. And then the mixture was filtered and the filtrate was concentrated to dryness under reduced pressure. The crude mixture was purified by flash chromatography to afford 7 (5 g, 59%) as a yellow oil. MS m/z (ESI): 295.2 [M+H-100].

1H NMR (400 MHz, DMSO) δ 7.42-7.09 (m, 5H), 5.12-4.93 (m, 2H), 4.92-4.67 (m, 1H), 4.59-4.58 (M, 2H), 3.70-3.48 (m, 4H), 3.28-3.13 (m, 2H), 2.40-2.22 (m, 1H), 2.19-2.03 (m, 2H), 1.94-1.43 (m, 2H), 1.38-1.34 (m, 9H).

To a solution of 7 (5.7 g, 14.47 mmol) in DCM (60 mL) was added TBSCl (2.28 g, 15.19 mmol), Et₃N (1.54 g, 15.19 mmol) and DMAP (1.85 g, 15.19 mmol) at 0° C. The mixture was stirred for 16 h at rt. The reaction mixture was quenched by the addition of saturated aqueous NaHCO₃ (50 mL). The aqueous layer was extracted with EA (2×60 mL). The combined organic extracts were dried over Na₂SO₄. And then the mixture was filtered, and the filtrate was concentrated to dryness under reduced pressure. The crude mixture was purified by flash chromatography to afford 8 (2.3 g, 31%) as a yellow oil. MS m/z (ESI): 531.4 [M+23].

1H NMR (400 MHz, DMSO) δ 7.37-7.15 (m, 5H), 5.10-4.94 (m, 2H), 4.52-4.51 (m, 1H), 3.74-3.53 (m, 2H), 3.51-3.40 (m, 2H), 3.37-3.30 (m, 1H), 3.21-3.20 (m, 2H), 2.29-2.28 (m, 0.5H), 2.07-2.06 (m, 2H), 1.85-1.84 (m, 0.5H), 1.70-1.44 (m, 1H), 1.39-1.21 (m, 9H), 0.83-0.82 (m, 9H), 0.03-0.05 (m, 6H).

To a solution of 8 (300 mg, 0.59 mmol) in THF (18 mL) was added TsCl (224 mg, 1.18 mmol) and NaH (118 mg, 2.95 mmol) at 0° C. The mixture was stirred for 5 h at rt. The reaction mixture was quenched by the water (3 mL). The aqueous layer was extracted with EA (2×20 mL). The combined organic extracts were dried over Na₂SO₄. And then the mixture was filtered, and the filtrate was concentrated to dryness under reduced pressure. The crude mixture was purified by flash chromatography to afford 9 (160 mg, 55%) as a yellow oil. MS m/z (ESI): 491.4 [M+H]⁺.

1H NMR (400 MHz, DMSO) δ 7.34-7.33 (m, 5H), 5.20-4.99 (m, 2H), 3.98-3.97 (m, 1H), 3.85-3.40 (m, 6H), 2.34-1.80 (m, 4H), 1.42-1.27 (m, 9H), 0.87-0.80 (m, 9H), 0.12-−0.04 (m, 6H).

To a mixture of 9 (200 mg, 0.408 mmol) in THF (5 mL) was added TBAF (0.8 mL, 0.816 mmol). The mixture was stirred for 4 h at rt. The reaction mixture was quenched by the addition of saturated aqueous NH₄Cl (10 mL). The aqueous layer was extracted with EA (2×10 mL). The combined organic extracts were dried over Na₂SO₄. And then the mixture was filtered and the filtrate was concentrated to dryness under reduced pressure. The crude mixture was purified by flash chromatography to afford 10 (110 mg, 72%) as a yellow oil. MS m/z (ESI): 399.2 [M+23].

1H NMR (400 MHz, CDCl₃) δ 7.37-7.36 (m, 5H), 5.15 (s, 2H), 4.04-3.49 (m, 8H), 2.30-2.07 (m, 4H), 1.52-1.34 (m, 9H).

To a mixture of 10 (300 mg, 0.798 mmol) in DCM (3 mL) and H₂O (3 mL) was added CH₃COOK (469 mg, 4.788 mmol) and (Bromodifluoromethyl)trimethylsilane (644 mg, 3.192 mmol) at 0° C. The mixture was stirred for 16 h at rt. The reaction mixture was quenched by the addition of saturated aqueous NaHCO₃ (5 mL). The aqueous layer was extracted with EA (2×10 mL). The combined organic extracts were dried over Na₂SO₄. And then the mixture was filtered and the filtrate was concentrated to dryness under reduced pressure. The crude mixture was purified by flash chromatography to afford 11 (150 mg, 44%) as a yellow oil. MS m/z (ESI): 427.2 [M+H]⁺.

1H NMR (400 MHz, DMSO) δ 7.35-7.34 (m, 5H), 6.92-6.38 (m, 1H), 5.20-4.99 (m, 2H), 4.12-4.11 (m, 1H), 3.91-3.90 (m, 2H), 3.80-3.36 (m, 4H), 2.63-2.62 (m, 1H), 2.32-2.03 (m, 3H), 1.46-1.23 (m, 9H).

To a solution of 11 (140 mg, 0.023 mmol) in EA (2 mL) was added Pd/C (140 mg). The mixture was stirred for 2 h at rt. The mixture was filtered. The filtrate was concentrated to dryness under reduced pressure to afford 12 (80 mg, 83%) as a yellow oil. MS m/z (ESI): 293.5 [M+H]⁺.

To a mixture of 12 (108 mg, 0.369 mmol) in DMF (5 mL) was added 13 (180 mg, 0.279 mmol), PyBop (435 mg, 0.837 mmol) and DIPEA (179 mg, 1.395 mmol). The mixture was stirred for 1h at rt. The reaction mixture was quenched with water (5 mL). The aqueous layer was extracted with EA (2×20 mL). The combined organic extracts were dried over Na₂SO₄ and concentrated in vacuo. The crude mixture was purified by flash chromatography to afford 14 (200 mg, 59%) as a yellow solid. MS m/z (ESI): 920.2 [M+H]⁺.

1H NMR (400 MHz, DMSO) δ 8.14-7.93 (m, 1H), 7.31-7.20 (m, 1H), 6.95-6.45 (m, 1H), 5.32-5.31 (m, 2H), 4.88-4.06 (m, 6H), 3.59-3.58 (m, 3H), 3.02-3.01 (m, 3H), 2.61-2.60 (m, 2H), 2.49-2.06 (m, 6H), 1.87-1.86 (m, 3H), 1.36-1.35 (m, 18H).

To a mixture of 14 (80 mg, 0.196 mmol) in DCM (2 mL) was added TFA (2 mL). The mixture was stirred for 1h at rt. The reaction mixture was quenched NaHCO₃ (15 mL). The aqueous layer was extracted with EA (2×20 mL). The combined organic extracts were dried over Na₂SO₄ and concentrated in vacuo. The crude mixture was purified by flash chromatography to afford 15 (50 mg, 79%) as a yellow solid. MS m/z (ESI): 720.2 [M+H]⁺.

To a mixture of 15 (50 mg, 0.069 mmol) in DMF (1 mL) was added di(1H-1,2,4-triazol-1-yl)methanone (35 mg, 0.208 mmol) and DIPEA (30 mg, 0.208 mmol). The mixture was stirred for 1h at rt. The crude was purified by prep-HPLC to give GD-XL0841-P1 (6.06 mg) and 327 (7.06 mg) as a white solid. MS m/z (ESI): 815.3 [M+H]⁺.

1H NMR (400 MHz, DMSO) δ 9.11 (s, 1H), 8.31-8.03 (m, 4H), 7.19-7.17 (m, 2H), 6.70-6.50 (m, 1H), 5.28-5.20 (m, 2H), 4.69 (d, J=11.2 Hz, 1H), 4.59-4.41 (m, 3H), 4.19-4.15 (m, 2H), 4.13-3.96 (m, 2H), 3.04-2.97 (m, 4H), 2.86-2.65 (m, 2H), 2.46-2.25 (m, 2H), 2.09-2.01 (m, 3H), 1.89-1.70 (m, 3H).

### Example AC: Synthesis of (4R)-2-amino-4-(6-chloro-4-(8-(cyanomethyl)-1-(3-(difluoromethyl)-1H-1,2,4-triazole-1-carbonyl)-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile 330

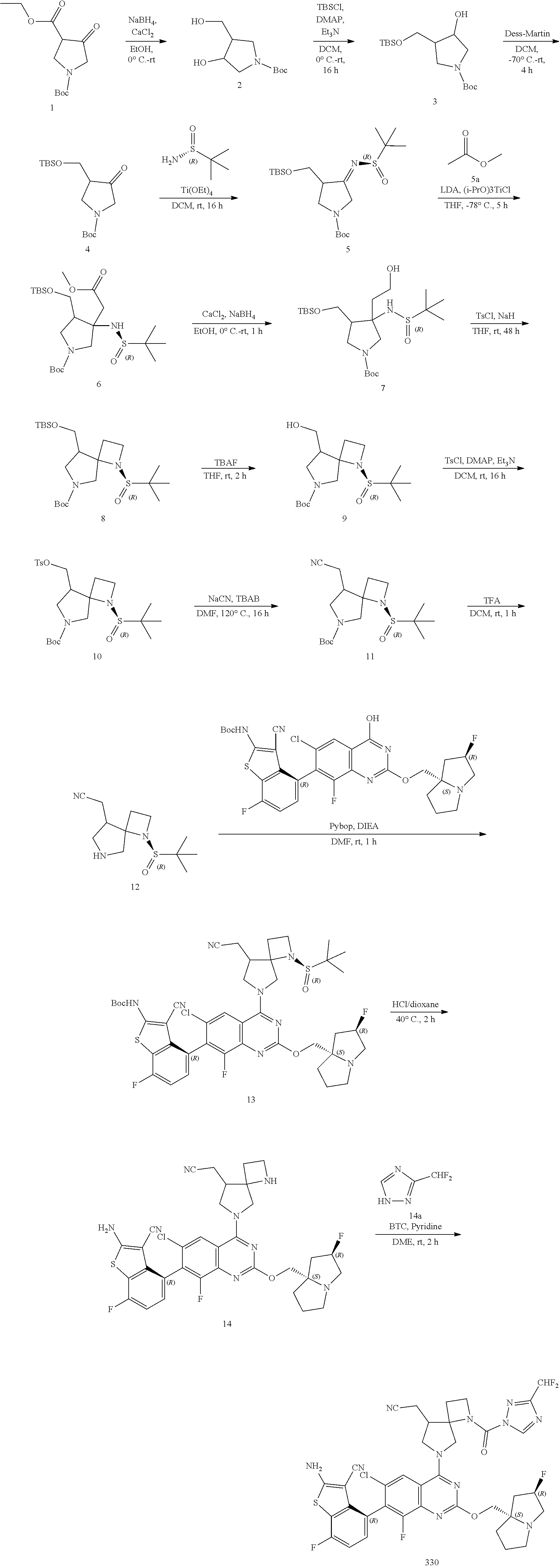

To a solution of 1 (20 g, 77.73 mmol) in EtOH (200 ml) was added NaBH₄ (29.4 g, 776 mmol) at 0° C. The reaction mixture was stirred for 15 h at rt. The mixture was quenched with ice water (30 mL) and extracted with EA (3×100 mL). The organic layer was washed with brine, dried over sodium sulfate, filtered, and concentrated to dryness under reduced pressure to give the crude, and purified by SGC (PE:EA=1:1) to get compound 2 (10 g, Yield: 59%) as a yellow oil.

1H NMR (400 MHz, DMSO) δ 4.71-4.64 (m, 2H), 4.13-4.12 (m, 0.5H), 3.96-3.94 (m, 0.5H), 3.44-3.31 (m, 3H), 3.29-3.16 (m, 1H), 3.11-2.87 (m, 2H), 2.19-2.02 (m, 1H), 1.39 (s, 9H).

To a solution of 2 (10.5 g, 48.33 mmol) in DCM (100 mL) was added TBSCl (7.28 g, 48.33 mmol), TEA (4.89 g, 48.33 mmol) and DMAP (5.96 g, 48.33 mmol) at 0° C. The reaction mixture was stirred for 3 h at rt. Then the reaction mixture was quenched with a.q NH₄Cl (100 mL) and extracted with DCM (3×100 mL). The organic layer was washed with brine, dried over sodium sulfate, filtered, and concentrated to dryness under reduced pressure to give the crude, and purified by SGC (PE:EA=3:1) to get compound 3 (7.5 g, Yield: 46.8%) as a yellow oil.

1H NMR (400 MHz, DMSO) δ 4.83-4.82 (m, 1H), 4.09-4.08 (m, 0.5H), 3.93-3.92 (m, 0.5H), 3.54-3.52 (m, 1H), 3.34-3.30 (m, 3H), 3.19-2.90 (m, 2H), 2.22-2.01 (m, 1H), 1.35 (s, 9H), 0.82-0.80 (m, 9H), 0.07-−0.04 (m, 6H).

To a solution of 3 (10 g, 30.16 mmol) in DCM (100 mL) was added Dess-Martin (19.19 g, 45.24 mmol) at 0° C. The reaction mixture was stirred for 2 h at rt. The reaction was quenched with a.q Na₂SO₃ (100 mL) and extracted with DCM (3×100 mL). The mixture was washed with brine, dried over sodium sulfate, filtered, and concentrated to dryness under reduced pressure to give the crude, and purified by SGC to afford compound 4 (7.3 g, Yield: 73%) as a yellow oil.

1H NMR (400 MHz, DMSO) δ 3.88-3.66 (m, 4H), 3.52-3.50 (m, 2H), 2.78-2.77 (m, 1H), 1.41 (s, 9H), 0.87-0.75 (m, 9H), 0.02-0.01 (m, 6H).

To a solution of 4 (2 g, 6.079 mmol) in THF (35 mL) was added SMI (1.1 g, 9.119 mmol) and Ti (OEt)₄(4.15 g, 18.237 mmol). The mixture was stirred for 2 h at 80° C. The reaction mixture was diluted with a.q NaHCO₃ (40 mL) and extracted with EA (50 mL*3). The mixture was washed with brine, dried over sodium sulfate, filtered, concentrated to dryness under reduced pressure to give the crude and purified by SGC to afford 5 (1.3 g, 50%) as a yellow oil. MS m/z (ESI): 433.4[M+H]+

1H NMR (400 MHz, CDCl₃) δ 4.36-4.33 (m, 1H), 4.08-4.06 (m, 1H), 3.99-3.59 (m, 3H), 3.44-3.42 (m, 0.5H), 2.97-2.95 (m, 0.5H), 1.64-1.41 (m, 10H), 1.38-1.18 (m, 9H), 0.91-0.76 (m, 9H), 0.12-−0.04 (m, 6H).

To a solution of n-butyllithium (2.5 M in hexanes, 1.13 mL, 2.829 mmol) was added dropwise at 0° C. to a solution of Diisopropylamine (291 mg, 2.887 mmol) in THF (5 mL) and the mixture solution stirred at 0° C. for 0.5 h. The reaction mixture was then cooled to −78° C. followed by dropwise addition of a solution of 5a (85 mg, 1.155 mmol) in THF (1 mL). The resulting reaction mixture was stirred at −78° C. for 1.5 h. After this time, a solution of (i-PrO)₃TiCl (3.46 mL, 3.465 mmol) was added dropwise and the reaction stirred for 1 h. A solution of sulfonamide 5 (500 mg, 1.155 mmol) in THF (2 mL) was then added dropwise and the reaction stirred for an additional 1 h. The reaction mixture was diluted with a.q NH₄Cl (10 mL) and extracted with EA (20 mL*3). The combined organic layers were washed with brine, dried over sodium sulfate, filtered, concentrated to dryness under reduced pressure to give the crude and purified by SGC to afford 6 (150 mg, 26%) as a yellow oil. MS m/z (ESI): 451.2 [M+H-56]+

To a mixture of 6 (150 mg, 0.296 mmol) in EtOH (5 mL) was added CaCl₂ (66 mg, 0.593 mmol) and NaBH₄ (45 mg, 1.186 mmol) at 0° C. The mixture was stirred for 16 h at rt. After then the reaction mixture was quenched with a.q Citric acid (10 mL/1 g/mL). The aqueous layer was extracted with EA (2×20 mL). The combined organic layers were washed with brine, dried over sodium sulfate, filtered, concentrated to dryness under reduced pressure to give the crude and purified by SGC to afford 7 (30 mg, 22%) as a white solid. MS m/z (ESI): 423.2 [M+H-56]+

To a solution of 7 (500 mg, 1.046 mmol) in THF (10 mL) was added TsCl (397 mg, 2.092 mmol) and NaH (209 mg, 5.23 mmol) at 0° C. The mixture was stirred for 16 h at rt. The reaction mixture was quenched with water (20 mL). The combined organic layer was extracted with EA (2×20 mL). The combined organic layers were washed with brine, dried over sodium sulfate, filtered, concentrated to dryness under reduced pressure to give the crude and purified by flash chromatography to afford 8 (170 mg, 35%) as a yellow oil. MS m/z (ESI): 405.2 [M+H-56]+.

1H NMR (400 MHz, DMSO) δ 4.17-3.83 (m, 3H), 3.67-3.65 (m, 2H), 3.34-3.33 (m, 1H), 3.26-3.11 (m, 2H), 2.19-2.18 (m, 1H), 1.96-1.95 (m, 2H), 1.45-1.29 (m, 9H), 1.04-1.03 (m, 9H), 0.84-0.82 (m, 9H), 0.02-0.01 (m, 6H).

To a solution of 8 (900 mg, 1.957 mmol) in THF (8 mL) was added TBAF (3.9 mL, 3.913 mmol). The mixture was stirred for 4 h at rt. The reaction mixture was quenched by the addition of saturated aqueous NH₄Cl (10 mL). The aqueous layer was extracted with EA (2×30 mL). The combined organic layers were washed with brine, dried over sodium sulfate, filtered, concentrated to dryness under reduced pressure to give the crude and purified by flash chromatography to afford 9 (500 mg, 87%) as a yellow oil. MS m/z (ESI): 291.1 [M+H-56]+.

1H NMR (400 MHz, DMSO) δ 4.71-4.70 (m, 1H), 4.10-3.82 (m, 2H), 3.57-3.55 (m, 1H), 3.36-3.35 (m, 1H), 3.30-3.24 (m, 1H), 3.23-3.15 (m, 2H), 2.62-2.60 (m, 1H), 2.17-2.16 (m, 1H), 1.99-1.98 (m, 1H), 1.41-1.38 (m, 9H), 1.06-1.03 (m, 9H).

To a solution of 9 (100 mg, 0.344 mmol) in DCM (5 mL) was added TsCl (78 mg, 0.413 mmol), DMAP (5 mg, 0.034 mmol) and TEA (104 mg, 1.032 mmol) at 0° C. The mixture was stirred for 16 h at rt. The reaction mixture was quenched with water (10 mL) and extracted with EA (3*10 mL). The combined organic layers were washed with brine, dried over sodium sulfate, filtered, concentrated to dryness under reduced pressure to give the crude and purified by flash chromatography to afford 10 (80 mg, 47%) as a yellow oil. MS m/z (ESI): 445.1 [M+H-56]+.

1H NMR (400 MHz, DMSO) δ 7.82-7.80 (m, 2H), 7.51-7.50 (m, 2H), 4.19-4.18 (m, 1H), 4.11-4.10 (m, 1H), 4.02-3.94 (m, 1H), 3.85-3.83 (m, 1H), 3.36-3.21 (m, 2H), 3.12-3.10 (m, 1H), 3.01-3.00 (m, 1H), 2.46-2.45 (m, 1H), 2.37-2.36 (m, 1H), 1.93-1.85 (m, 1H), 1.34-1.30 (m, 9H), 1.16-0.90 (m, 9H).

To a solution of 10 (600 mg, 1.2 mmol) in DMF (10 mL) was added NaCN (118 mg, 2.4 mmol) and TBAB (39 mg, 0.12 mmol). The mixture was stirred for 16 h at 120° C. The reaction mixture was quenched with water (10 mL) and extracted with EA (3*30 mL). The combined organic layers were washed with brine, dried over sodium sulfate, filtered, concentrated to dryness under reduced pressure to give the crude and purified by flash chromatography to afford 11 (400 mg, 94%) as a yellow oil. MS m/z (ESI): 356.3 [M+H]+.

To a solution of 11 (150 mg, 0.423 mmol) in DCM (2 mL) was added TFA (0.4 mL). The mixture was stirred for 1 h at rt. The reaction mixture was quenched with a.q NaHCO₃(10 mL). The aqueous layer was extracted with EA (2×20 mL). The combined organic layers were washed with brine, dried over sodium sulfate, filtered, concentrated to dryness under reduced pressure to give the crude and purified by flash chromatography to afford 12 (70 mg, crude) as a yellow solid.

MS m/z (ESI): 256.2 [M+H]+.

To a solution of 12 (51 mg, 0.202 mmol) in DMF (2 mL) was added 12a (100 mg, 0.155 mmol), PyBop (242 mg, 0.465 mmol) and DIPEA (100 mg, 0.775 mmol). The reaction mixture was stirred for 1 h at rt. The reaction mixture was quenched with water (5 mL). The aqueous layer was extracted with EA (2×20 mL). The combined organic layers were washed with brine, dried over sodium sulfate, filtered, concentrated to dryness under reduced pressure to give the crude and purified by flash chromatography to afford 13 (60 mg, 44%) as a yellow oil.

MS m/z (ESI): 883.4 [M+H]+.

To a solution of 13 (60 mg, 0.068 mmol) in HCl in dioxane (2 mL). The reaction mixture was stirred for 1 h at 40° C. The reaction mixture was quenched with a.q NaHCO₃(10 mL). The aqueous layer was extracted with EA (2×20 mL). The combined organic layers were washed with brine, dried over sodium sulfate, filtered, concentrated to dryness under reduced pressure to give the crude and purified by flash chromatography to afford 14 (35 mg, crude) as a yellow solid.

MS m/z (ESI): 679.2 [M+H]+.

To a solution of compound 14a (596 mg, 5.01 mmol) and Pyridine (1.58 g, 20.02 mmol) in ACN (17 mL) was added BTC (1.34 g, 4.51 mmol) at 0° C. under N₂ atmosphere. The reaction mixture was then stirred at 0° C. for 2 h. To a solution of compound 14 (50 mg, 0.074 mmol) in DME (0.5 mL) was added Pyridine (0.1 mL, 1.24 mmol) and above reaction mixture (0.15 mL) at rt under N₂ atmosphere. LCMS showed the reaction was completed. The mixture was poured into water (15 mL), and the solution was extracted with ethyl acetate (15 ml×3). The organic layer was washed with brine (15 mL), dried over anhydrous sodium sulfate, filtered and the filtrate was concentrated to dryness under reduced pressure. The crude was purified by prep-HPLC (FA) to give Yield: 330 (18.10 mg, 29.71%) as a white solid.

¹H NMR (400 MHz, DMSO) 9.40-9.32 (m, 1H), 8.15-8.05 (m, 3H), 7.34-7.01 (m, 3H), 5.27 (d, J=53.2 Hz, 1H), 4.80-4.71 (s, 1H), 4.65-4.48 (m, 2H), 4.44-4.19 (m, 2H), 4.15-4.05 (m, 1H), 4.03-3.95 (m, 1H), 3.90-3.75 (m, 1H), 3.51-3.46 (m, 1H), 3.20-2.99 (m, 6H), 2.86-2.75 (m, 1H), 2.45-2.32 (m, 1H), 2.15-2.01 (m, 3H), 1.90-1.70 (m, 3H).

### Example AD: Synthesis of 2-Amino-4-(6-chloro-8-fluoro-2-(((2R,7aS)-2-fluorotetrahydro-1H-pyrrolizin-7a(5H)-yl)methoxy)-4-(6-(3-isopropyl-5-methyl-1H-1,2,4-triazole-1-carbonyl)-9-oxa-2,6-diazaspiro[4.5]decan-2-yl)quinazolin-7-yl)-7-fluorobenzo[b]thiophene-3-carbonitrile (337)

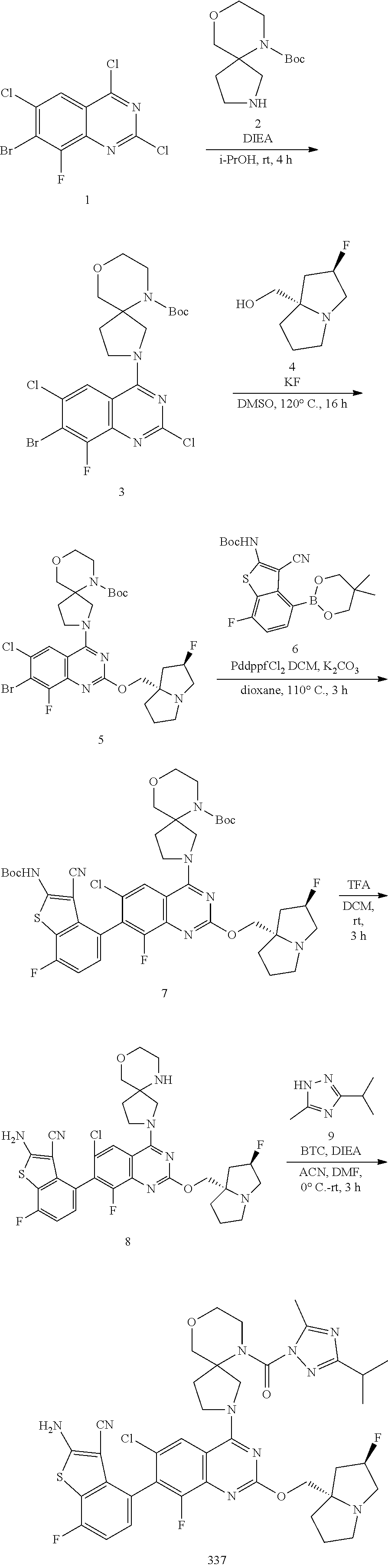

To a solution of compound 1 (680 mg, 2.06 mmol) and compound 2 (499 mg, 2.06 mmol) in i-PrOH (8 ml) was added DIEA (799 mg, 6.18 mmol). The reaction mixture was stirred at 25° C. for 4 h. The mixture was poured into ice water (100 mL), and the solution was extracted with ethyl acetate (100 ml×3). The organic layer was washed with brine (100 mL), dried over anhydrous sodium sulfate. The organic solution was concentrated to dryness under reduced pressure to give compound 3 (1.0 g, Yield: 90.9%) as a yellow solid.

MS m/z (ESI): 535.1 [M+H]⁺.

To a solution of compound 3 (950 mg, 1.77 mmol) and compound 4 (1.41 g, 8.86 mmol) in DMSO (10 ml) was added KF (514 mg, 8.86 mmol). The reaction mixture was stirred at 120° C. for 16 h. The mixture was poured into ice water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 5 (400 mg, Yield: 34.4%) as a yellow solid.

MS m/z (ESI): 658.2 [M+H]⁺.

To a solution of compound 5 (400 mg, 0.61 mmol), compound 6 (410 mg, 1.22 mmol), and K₂CO₃ (506 mg, 3.66 mmol) in 1,4-dioxane (15 mL) was added PddppfCl₂with DCM (49 mg, 0.06 mmol). The mixture was stirred at 110° C. under nitrogen for 3 hours. The mixture was poured into water (100 mL), and the solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (PE/EA=1/1) to give compound 7 (430 mg, Yield: 81.13%) as a white solid. MS m/z (ESI): 870.3 [M+H]⁺.

To a solution of compound 7 (170 mg, 0.20 mmol) in DCM (10 ml) was added TFA (5 ml). The reaction mixture was stirred at rt for 3 h. The reaction mixture was concentrated to dryness under reduced pressure. The reaction mixture was diluted with a.q. NaHCO₃ (20 mL) and EA (50 mL). The organic layer was washed with brine, dried over sodium sulfate, filtered, and concentrated to dryness under reduced pressure to give compound 8 (130 mg, Yield: 99.3%) as a yellow solid.

MS m/z (ESI): 670.2 [M+H]⁺.

To a solution of compound 9 (93 mg, 0.75 mmol) and DIEA (0.5 ml, 3.025 mmol) in MeCN (4.5 ml) was added triphosgene (200 mg, 0.675 mmol) at 0° C. under N₂ atmosphere. The reaction mixture was then stirred at 20° C. for 2 h, then was added to the solution of compound 8 (50 mg, 0.07 mmol) in DMF (1 mL) at 20° C. under N₂ atmosphere. The crude was purified by prep-HPLC (FA) to give 337 as a white solid (4.96 mg, Yield: 8.1%). MS m/z (ESI): 821.4 [M+H]⁺.

¹H NMR (400 MHz, DMSO) δ 8.15 (s, 1H), 8.11 (s, 2H), 7.27-7.20 (m, 1H), 7.15 (t, J=8.6 Hz, 1H), 5.27 (d, J=54.0 Hz, 1H), 4.48-4.31 (m, 1H), 4.32-4.05 (m, 4H), 4.04-3.97 (m, 1H), 3.86-3.54 (m, 7H), 3.14-3.05 (m, 2H), 3.03-2.89 (m, 2H), 2.85-2.76 (m, 1H), 2.28 (s, 1H), 2.19-1.95 (m, 3H), 1.89-1.71 (m, 3H), 1.23 (d, J=64.0 Hz, 6H),

### Example AE: Synthesis of (R)-2-amino-4-(6-chloro-8-fluoro-2-(((2R,7aS)-2-fluorotetrahydro-1H-pyrrolizin-7a(5H)-yl)methoxy)-4-((R)-1-(3-methyl-1H-1,2,4-triazole-1-carbonyl)-1,6-diazaspiro[3.4]octan-6-yl)quinazolin-7-yl)-7-fluorobenzo[b]thiophene-3-carbonitrile 338

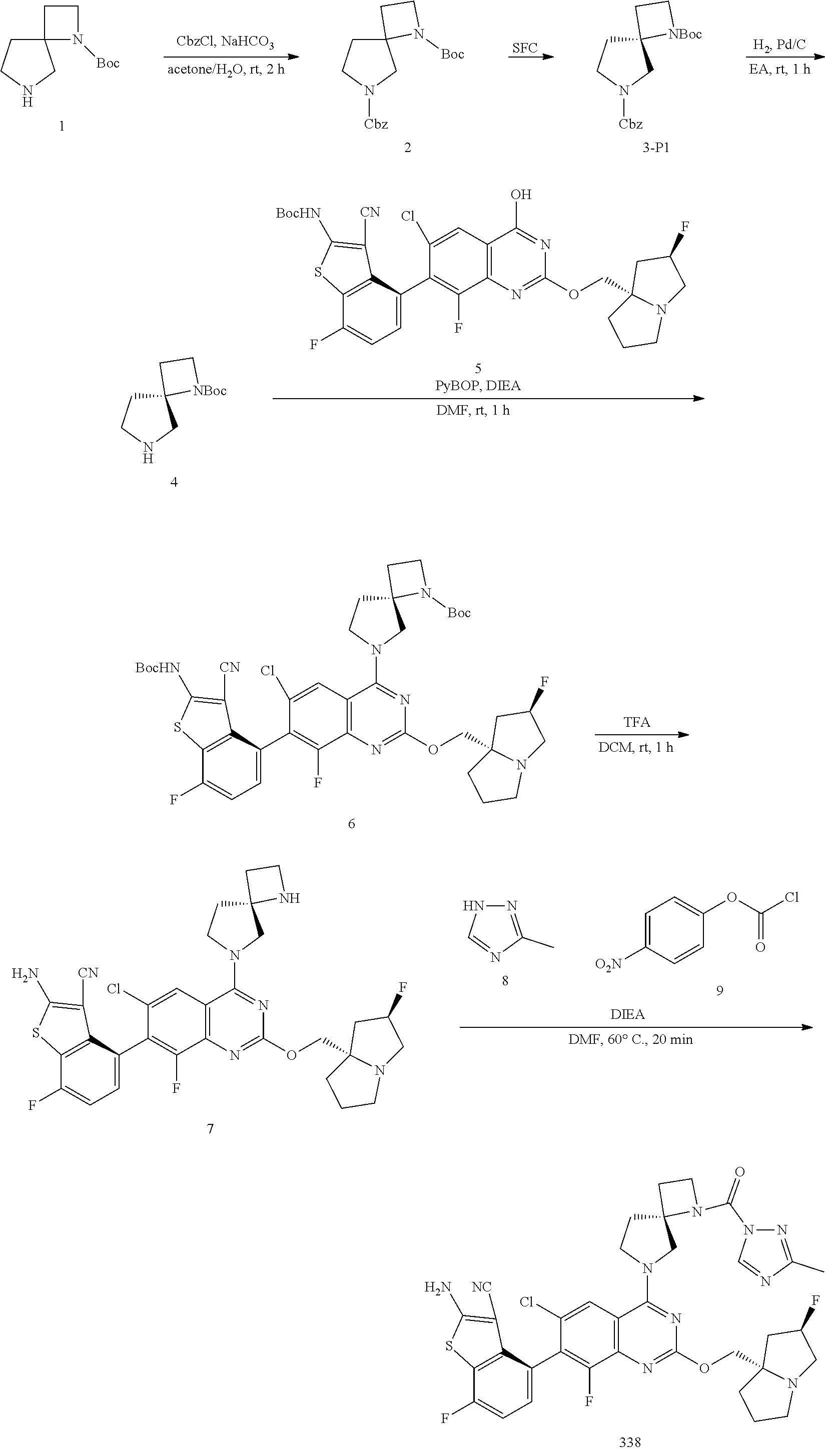

To a solution of compound 1 (4 g, 7.78 mmol) and NaHCO₃ (2.61 g, 31.12 mmol) in acetone (20 ml) was added H₂O (10 ml). The reaction mixture was added CbzCl (2.65 g, 15.56 mmol) and stirred at 25° C. for 1 h. The solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=2/1) to give compound 2 (4 g, Yield: 74%) as colorless oily liquid.

¹H NMR (400 MHz, CDCl₃) δ 7.40-7.26 (m, 5H), 5.40-4.94 (m, 2H), 4.22-3.12 (m, 6H), 2.55-2.25 (m, 1H), 2.31-2.04 (m, 2H), 1.96 (s, 1H), 1.55-1.23 (m, 9H).

Compound 2 (4 g, 11.55 mmol) was purified by SFC (solvent: EtOH) to give compound 3-P1 (1.8 g) as colorless oily liquid and compound 3-P2 (1.6 g) as colorless oily liquid.

To a solution of compound 3-P1 (1.5 g, 4.33 mmol) in ethyl acetate (50 ml) was added Pd/C (750 mg). The reaction mixture was stirred at 25° C. under H₂ atmosphere for 1 h. The reaction mixture was diluted with ethyl acetate (50 ml) and methanol (100 ml). The organic layer was filtered, and concentrated to afford compound 4 (780 mg, 85%) as colorless oily liquid.

¹H NMR (400 MHz, CDCl₃) δ 4.04-3.65 (m, 2H), 3.54-3.05 (m, 2H), 2.95-2.62 (m, 2H), 2.42-2.17 (m, 4H), 1.91 (m, 1H), 1.65-1.35 (m, 911).

To a solution of compound 4 (299 mg, 1.41 mmol), compound 5 (700 mg, 1.09 mmol), and PyBop (847 mg, 1.63 mmol) in DMF (5 mL) was added DIEA (210 mg, 1.63 mmol). The mixture was stirred at 25° C. for 1 h. The solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=15/1) to give compound 6 (700 mg, Yield: 77%) as a yellow solid. MS m/z (ESI): 840.3 [M+H]⁺.

To a solution of compound 6 (1.3 g, 1.55 mmol) in DCM (5 ml) was added TFA (5 ml). The reaction mixture was stirred at 20° C. for 30 min. The solvent was removed under reduced pressure. The reaction mixture was diluted with a.q. NaHCO₃ (200 mL) and DCM/MeOH=10/1 (300 mL). The organic layer was washed with brine, dried over sodium sulfate, filtered, and concentrated to give compound 7 (830 mg, Yield: 84%) as a yellow solid. MS m/z (ESI): 640.6 [M+H]⁺.

To a solution of compound 8 (527 mg, 6.35 mmol) and DIEA (1.23 g, 9.53 mmol) in 10 mL MeCN, was added 4-nitrophenyl carbonochloridate (1.41 g, 6.99 mmol) at 0° C. under N₂ atmosphere. The reaction mixture was then stirred at rt for 1 h. To a solution of compound 7 (40 mg, 0.063 mmol) in DMF (0.5 mL) was added above reaction mixture (0.15 mL) at 60° C. under N₂ atmosphere.

The solution was stirred at 60° C. for 20 min. The mixture was poured into ice water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by Pre-HPLC (FA) to give 338 (14.4 mg, Yield: 30.55%) as a white solid.

¹H NMR (400 MHz, DMSO) δ 8.98 (s, 1H), 8.20-8.05 (m, 3H), 7.29-7.20 (m, 1H), 7.18-7.10 (m, 1H), 5.27 (d, J=54.0 Hz, 1H), 4.70-4.48 (m, 3H), 4.37-3.84 (m, 6H), 3.13-3.00 (m, 3H), 2.90-2.69 (m, 3H), 2.33 (s, 3H), 2.25-1.60 (m, 7H).

### Example AF: Synthesis of (4S)-2-amino-4-(6-chloro-4-((4R)-1-(2,2-difluorocyclopropane-1-carbonyl)-1,6-diazaspiro[3.4]octan-6-yl)-8-fluoro-2-((S)-1-((S)-1-methylpyrrolidin-2-yl)ethoxy)quinazolin-7-yl)-7-fluorobenzo[b]thiophene-3-carbonitrile (346)

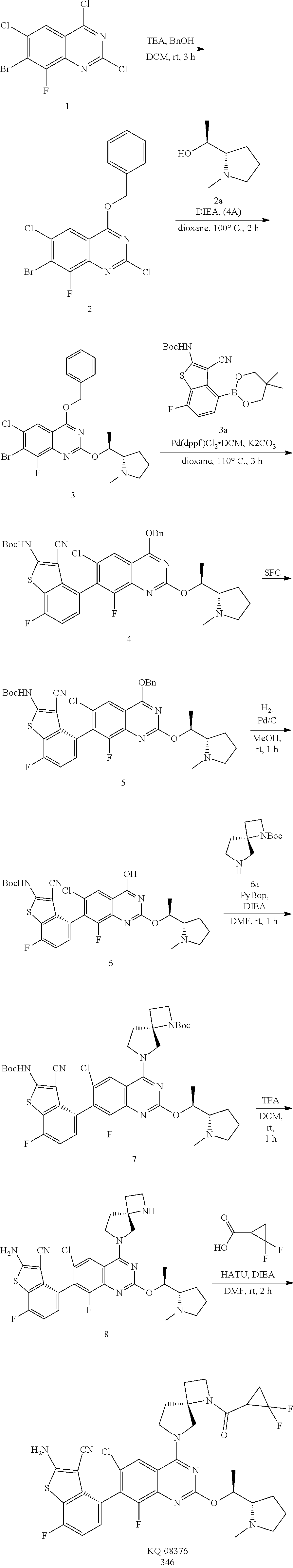

To a solution of compound 1 (5 g, nol) and TEA (4.59 g, 45.40 mmol) in DCM (50 mL) was added BnOH (1.96 g, 18.2 mmol). The mixture was stirred room temperature for 3 h. The mixture was poured into water (100 mL), and the solution was extracted with DCM (100 mL×3). The organic layer was washed with brine (50 mL), dried over anhydrous sodium sulfate, filtered and the filtrate was concentrated to dryness under reduced pressure, which was purified by silica gel column (PE/EA=10/1) to give compound 2 (4 g, Yield: 65.7%) as a yellow solid. MS m/z (ESI): 401.0 [M+H].

A mixture of compound 2 (5 g, 12.44 mmol), compound 2a (4.82 g, 37.31 mmol), molecular sieve and (4A) (500 mg), DIEA (4.82 g, 37.31 mmol) in dioxane (30 ml) was stirred at 100° C. for 2 h under N₂. The mixture was filtered, and the filtrate was extracted with EA (100 mL), washed with brine (50 mL×3), dried over anhydrous sodium sulfate, filtered and the filtrate was concentrated to dryness under reduced pressure. The residue was purified by silica gel column (PE/EA=1/1) to give compound 3 (2.5 g, Yield: 40.7%) as a white solid. MS m/z (ESI): 494.0 [M+H].

To a solution of compound 3 (2 g, 4.05 mmol), compound 3a (2.42 g, 6.0 mmol) and K₂CO₃(3.36 g, 24.3 mmol) in dioxane (30 mL) was added Pd(dppf)Cl₂-DCM (330 mg, 0.4 mmol). The mixture was stirred 100° C. for 3 h under N₂. The mixture was poured into water (100 mL), and the solution was extracted with ethyl acetate (100 mL×3). The organic layer was washed with brine (50 mL), dried over anhydrous sodium sulfate, filtered and the filtrate was concentrated to dryness under reduced pressure. The residue was purified by silica gel column (MeOH/DCM=15/1) to get compound 4 (0.8 g, Yield: 28%) as a yellow solid. MS m/z (ESI): 706.2 [M+H]. Compound 4 (0.5 g, 0.7 mmol) was purified by SFC (solvent: EtOH) to obtain compound 5-P1 (0.2 g) and compound 5-P2 (0.2 g) as yellow solid.

To a solution of compound 5-P1 (0.17 g, 0.24 mmol) in MeOH (5 mL) was added Pd/C (30 mg). The mixture was stirred 25° C. for 0.5 h under H₂ atmosphere. The mixture was filtered, and the filtrate was concentrated to dryness under reduced pressure to get compound 6 (0.13 g, Yield: 88%) as a grey solid. MS m/z (ESI): 616.3 [M+H].

To a solution of compound 6 (100 mg, 0.16 mmol), compound 6a (45 mg, 0.21 mmol) and PyBop (125 mg, 0.24 mmol) in DMF (2 mL) was added DIEA (31 mg, 0.24 mmol). The mixture was stirred at room temperature for 1 h. The mixture was poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by prep-TLC (MeOH:DCM=15:1) to get compound 7 (70 mg, Yield: 54.0%) as a yellow solid. MS m/z (ESI): 810.4 [M+H].

To a solution of compound 7 (100 mg, 0.12 mmol) in DCM (2 ml) was added TFA (1 ml). The reaction mixture was stirred at 20° C. for 1 h. The solvent was removed under reduced pressure. The reaction mixture was diluted with a.q. NaHCO₃ (20 mL) and DCM (50 mL). The organic layer was washed with brine, dried over sodium sulfate, filtered, and concentrated to afford compound 8 (65 mg, 88.8%) as a yellow solid. MS m/z (ESI): 610.2 [M+H].

To a solution of compound 8 (100 mg, 0.8 mmol) and DIEA (207 mg, 1.6 mmol) in 5 ml DMF was added HATU (456 mg, 1.2 mmol) at 0° C. The reaction mixture was then stirred at 25° C. for 30 min. To a solution of compound 8 (45 mg, 0.057 mmol) in DMF (1 mL) was added above reaction mixture (0.45 mL). The mixture was stirred at 25° C. for 2 h, then poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by Pre-HPLC (FA) to give 346 (11.58 mg, Yield: 28.5%) as a white solid. MS m/z (ESI): 714.2 [M+H].

1H NMR (400 MHz, DMSO-d₆) δ 8.16 (s, 1H), 8.15-8.00 (m, 3H), 7.29-7.20 (m, 1H), 7.19-7.09 (m, 1H), 5.35-5.22 (m, 1H), 4.46-3.86 (m, 6H), 3.07-2.99 (m, 1H), 2.90-2.60 (m, 3H), 2.46 (s, 3H), 2.43-1.97 (m, 4H), 1.92-1.61 (m, 6H), 1.26 (d, J=6.4 Hz, 3H).

### Example AG: Synthesis of (R)-2-amino-4-(6-chloro-4-((R)-1-(2-cyanoacetyl)-1,6-diazaspiro[3.4]octan-6-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (350)

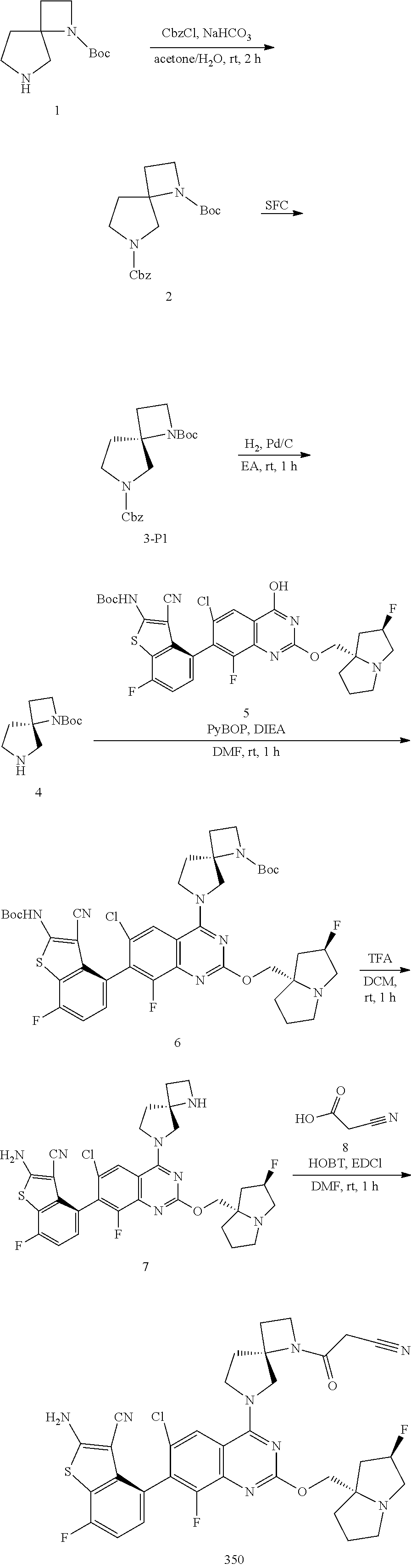

To a solution of compound 1 (4 g, 7.78 mmol) and NaHCO₃ (2.61 g, 31.12 mmol) in acetone (20 ml) was added H₂O (10 ml). The reaction mixture was added CbzCl (2.65 g, 15.56 mmol) stirred at 25° C. for 1 h. The solution was extracted with ethyl acetate (200 mL×3). The organic layer was washed with brine (100 mL), dried over anhydrous sodium sulfate, filtered and the filtrate was concentrated to dryness under reduced pressure. The residue was purified by silica gel column (PE/EA=2/1) to give compound 2 (4 g, Yield: 74%) as colorless oily liquid.

1H NMR (400 MHz, CDCl₃) δ 7.40-7.26 (m, 5H), 5.40-4.94 (m, 2H), 4.22-3.12 (m, 6H), 2.55-2.25 (m, 1H), 2.31-2.04 (m, 2H), 1.96 (s, 1H), 1.55-1.23 (m, 9H).

Compound 2 (4 g, 11.55 mmol) was purified by SFC (solvent: EtOH) to give compound 3-P1 (1.8 g) as colorless oily liquid and compound 3-P2 (1.6 g) as colorless oily liquid.

To a solution of compound 3-P1 (1.5 g, 4.33 mmol) in ethyl acetate (50 ml) was added Pd/C (750 mg). The reaction mixture was stirred at 25° C. under H₂ atmosphere for 1 h. The reaction mixture was diluted with ethyl acetate (50 ml) and methanol (100 ml). The organic layer was filtered, and concentrated to afford compound 4 (780 mg, 85%) as colorless oily liquid.

¹H NMR (400 MHz, CDCl₃) δ 4.04-3.65 (m, 2H), 3.54-3.05 (m, 2H), 2.95-2.62 (m, 2H), 2.42-2.17 (m, 4H), 1.91 (m, 1H), 1.65-1.35 (m, 911).

To a solution of compound 4 (299 mg, 1.41 mmol), compound 5 (700 mg, 1.09 mmol), and PyBop (847 mg, 1.63 mmol) in DMF (5 mL) was added DIEA (210 mg, 1.63 mmol). The mixture was stirred at 25° C. for 1 h. The solution was extracted with ethyl acetate (100 mL×3). The organic layer was washed with brine (100 mL), dried over anhydrous sodium sulfate, filtered and the filtrate was concentrated to dryness under reduced pressure. The residue was purified by silica gel column (DCM/MeOH=15/1) to give compound 6 (700 mg, Yield: 77%) as a yellow solid. MS m/z (ESI): 840.3 [M+H]⁺.

To a solution of compound 6 (1.3 g, 1.55 mmol) in DCM (5 ml) was added TFA (5 ml). The reaction mixture was stirred at 20° C. for 30 min. The solvent was removed under reduced pressure. The reaction mixture was diluted with a.q. NaHCO₃ (200 mL) and DCM/MeOH=10/1 (300 mL). The organic layer was washed with brine, dried over sodium sulfate, filtered, and concentrated to give compound 7 (830 mg, Yield: 84%) as a yellow solid. MS m/z (ESI): 640.6 [M+H]⁺.

To a solution of compound 7 (40 mg, 0.063 mmol), compound 8 (8 mg, 0.094 mmol), HOBT (25 mg, 0.188 mmol), and EDCI (36 mg, 0.188 mmol) in DMF (2 mL) was added DIEA (24 mg, 0.188 mmol). The mixture was stirred at 25° C. under nitrogen for 16 hours. The mixture was poured into water (50 mL), and the solution was extracted with ethyl acetate (50 mL×3). The organic layer was washed with brine (50 mL), dried over anhydrous sodium sulfate, filtered and the filtrate was concentrated to dryness under reduced pressure. The residue was purified by Prep-HPLC (FA) to give compound 350 (3.50 mg, Yield: 7.9%) as a white solid. MS m/z (ESI): 707.3 [M+H]⁺.

1H NMR (400 MHz, DMSO-d₆) δ 8.12 (d, J=10.8 Hz, 3H), 7.27-7.20 (m, 1H), 7.18-7.12 (m, 1H), 5.30 (d, J=53.2 Hz, 1H), 4.45 (d, J=13.2 Hz, 1H), 4.17-3.99 (m, 6H), 3.72 (m, 2H), 3.18-2.65 (m, 5H), 2.41-1.69 (m, 1011).

TABLE 1

| No. | Structure | Chemical Name | [M + H]⁺ |
|---|---|---|---|
| 201 | 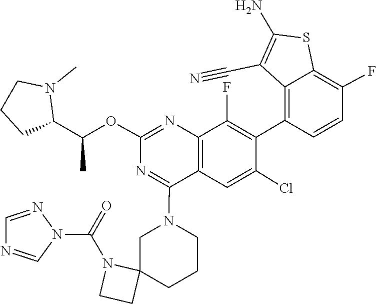 [SMILES: C[C@H](Oc1nc(N2CCCC3(CCN3C(=O)n3cncn3)C2)c2cc(Cl)c(-c3ccc(F)c4sc(N)c(C#N)c34)c(F)c2n1)[C@@H]1CCCN1C] | 4-(4-(1-(1H-1,2,4-triazole-1-carbonyl)-1,6- diazaspiro[3.5]nonan-6-yl)-6-chloro-8-fluoro-2- ((S)-1-((S)-1-methylpyrrolidin-2- yl)ethoxy)quinazolin-7-yl)-2-amino-7- fluorobenzo[b]thiophene-3-carbonitrile | 719.3 |
| 202 | 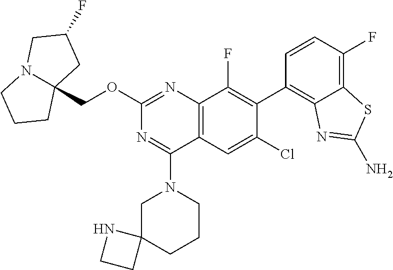 [SMILES: Nc1nc2c(-c3c(Cl)cc4c(N5CCCC6(CCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,6-diazaspiro[3.5]nonan-6- yl)quinazolin-7-yl)-7-fluorobenzo[d]thiazol-2- amine | 630.14 |
| 203 | 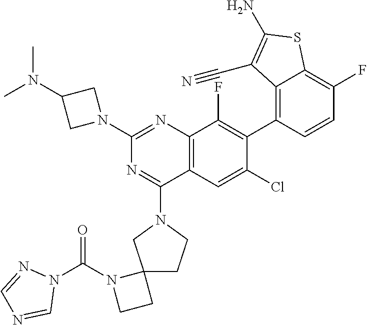 [SMILES: CN(C)C1CN(c2nc(N3CCC4(CCN4C(=O)n4cncn4)C3)c3cc(Cl)c(-c4ccc(F)c5sc(N)c(C#N)c45)c(F)c3n2)C1] | 4-(4-(1-(1H-1,2,4-triazole-1-carbonyl)-1,6- diazaspiro[3.4]octan-6-yl)-6-chloro-2-(3- (dimethylamino)azetidin-1-yl)-8- fluoroquinazolin-7-yl)-2-amino-7- fluorobenzo[b]thiophene-3-carbonitrile | 676.2 |
| 204 | 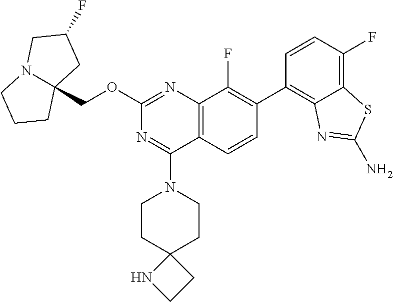 [SMILES: Nc1nc2c(-c3ccc4c(N5CCC6(CCN6)CC5)nc(OC[C@@]56CCCN5C[C@H](F)C6)nc4c3F)ccc(F)c2s1] | 7-fluoro-4-(8-fluoro-2-(((2R,7aS)-2- fluorotetrahydro-1H-pyrrolizin-7a(5H)- yl)methoxy)-4-(1,7-diazaspiro[3.5]nonan-7- yl)quinazolin-7-yl)benzo[d]thiazol-2-amine | 596.3 |
| 205 | 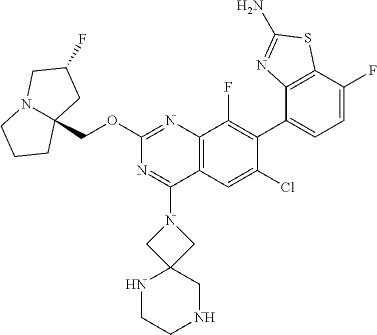 [SMILES: Nc1nc2c(-c3c(Cl)cc4c(N5CC6(CNCCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(2,5,8-triazaspiro[3.5]nonan-2- yl)quinazolin-7-yl)-7-fluorobenzo[d]thiazol-2- amine | 631.2 |
| 206 | 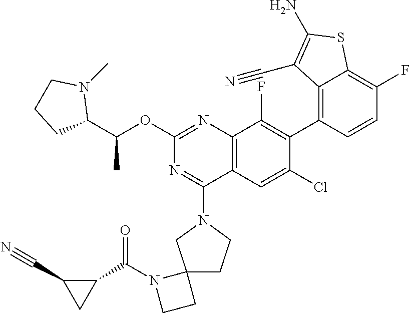 [SMILES: C[C@H](Oc1nc(N2CCC3(CCN3C(=O)[C@@H]3C[C@H]3C#N)C2)c2cc(Cl)c(-c3ccc(F)c4sc(N)c(C#N)c34)c(F)c2n1)[C@@H]1CCCN1C] | 2-amino-4-(6-chloro-4-(1-((1R,2R)-2- cyanocyclopropane-1-carbonyl)-1,6- diazaspiro[3.4]octan-6-yl)-8-fluoro-2-((S)-1- ((S)-1-methylpyrrolidin-2-yl)ethoxy)quinazolin- 7-yl)-7-fluorobenzo[b]thiophene-3-carbonitrile | 703.3 |
| 207 | 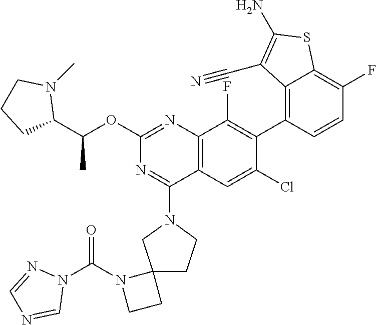 [SMILES: C[C@H](Oc1nc(N2CCC3(CCN3C(=O)n3cncn3)C2)c2cc(Cl)c(-c3ccc(F)c4sc(N)c(C#N)c34)c(F)c2n1)[C@@H]1CCCN1C] | 4-(4-(1-(1H-1,2,4-triazole-1-carbonyl)-1,6- diazaspiro[3.4]octan-6-yl)-6-chloro-8-fluoro-2- ((S)-1-((S)-1-methylpyrrolidin-2- yl)ethoxy)quinazolin-7-yl)-2-amino-7- fluorobenzo[b]thiophene-3-carbonitrile | 705.1 |
| 208 ¹ | 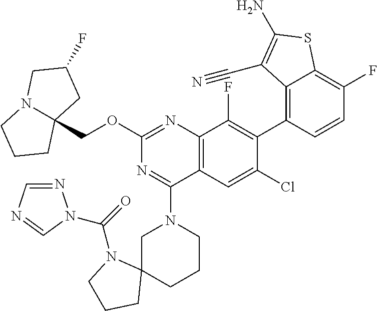 [SMILES: N#Cc1c(N)sc2c(F)ccc(-c3c(Cl)cc4c(N5CCCC6(CCCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,7- diazaspiro[4.5]decan-7-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 763.4 |
| 209 | 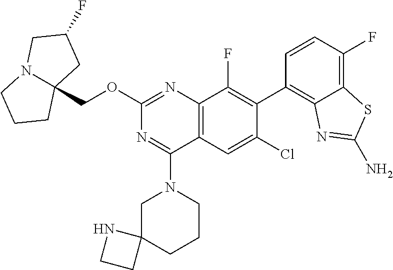 [SMILES: Nc1nc2c(-c3c(Cl)cc4c(N5CCCC6(CCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,6-diazaspiro[3.5]nonan-6- yl)quinazolin-7-yl)-7-fluorobenzo[d]thiazol-2- amine | 630.3 |
| 210 | 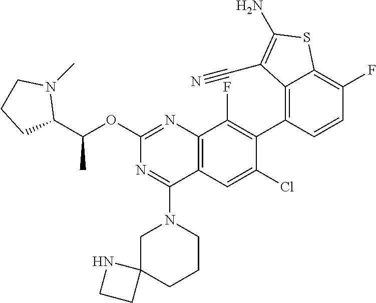 [SMILES: C[C@H](Oc1nc(N2CCCC3(CCN3)C2)c2cc(Cl)c(-c3ccc(F)c4sc(N)c(C#N)c34)c(F)c2n1)[C@@H]1CCCN1C] | 2-amino-4-(6-chloro-8-fluoro-2-((S)-1-((S)-1- methylpyrrolidin-2-yl)ethoxy)-4-(1,6- diazaspiro[3.5]nonan-6-yl)quinazolin-7-yl)-7- fluorobenzo[b]thiophene-3-carbonitrile | 624.5 |
| 211 | 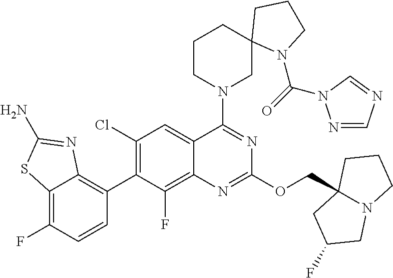 [SMILES: Nc1nc2c(-c3c(Cl)cc4c(N5CCCC6(CCCN6C(=O)n6cncn6)C5)nc(OC[C@@]56CCCN5C[C@H](F)C6)nc4c3F)ccc(F)c2s1] | (7-(7-(2-amino-7-fluorobenzo[d]thiazol-4-yl)-6- chloro-8-fluoro-2-(((2R,7aS)-2- fluorotetrahydro-1H-pyrrolizin-7a(5H)- yl)methoxy)quinazolin-4-yl)-1,7- diazaspiro[4.5]decan-1-yl)(1H-1,2,4-triazol-1- yl)methanone | 739.3 |
| 212 | 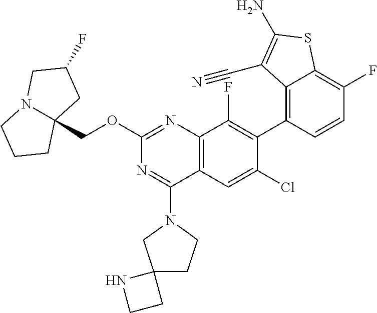 [SMILES: N#Cc1c(N)sc2c(F)ccc(-c3c(Cl)cc4c(N5CCC6(CCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1,6-diazaspiro[3.4]octan-6- yl)quinazolin-7-yl)-7-fluorobenzo[b]thiophene- 3-carbonitrile | 640.2 |
| 213 | 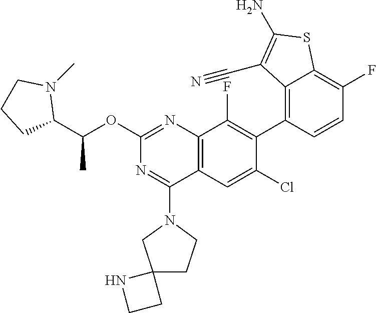 [SMILES: C[C@H](Oc1nc(N2CCC3(CCN3)C2)c2cc(Cl)c(-c3ccc(F)c4sc(N)c(C#N)c34)c(F)c2n1)[C@@H]1CCCN1C] | 2-amino-4-(6-chloro-8-fluoro-2-((S)-1-((S)-1- methylpyrrolidin-2-yl)ethoxy)-4-(1,6- diazaspiro[3.4]octan-6-yl)quinazolin-7-yl)-7- fluorobenzo[b]thiophene-3-carbonitrile | 610.3 |
| 214 ⁴ | 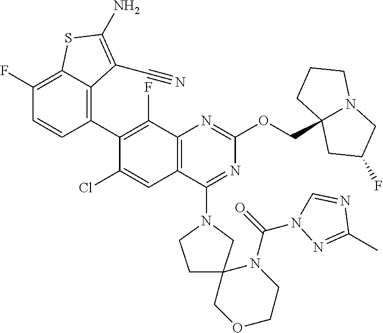 [SMILES: Cc1ncn(C(=O)N2CCOCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-(6-(3-methyl-1H-1,2,4-triazole- 1-carbonyl)-9-oxa-2,6-diazaspiro[4.5]decan-2- yl)quinazolin-7-yl)-7-fluorobenzo[b]thiophene- 3-carbonitrile | 779.3 |
| 215 | 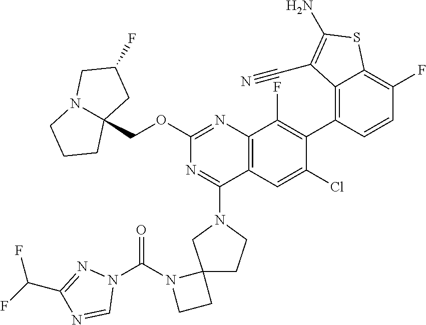 [SMILES: N#Cc1c(N)sc2c(F)ccc(-c3c(Cl)cc4c(N5CCC6(CCN6C(=O)n6cnc(C(F)F)n6)C5)nc(OC[C@@]56CCCN5C[C@H](F)C6)nc4c3F)c12] | 2-amino-4-(6-chloro-4-(1-(3-(difluoromethyl)- 1H-1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 785.5 |
| 216 | 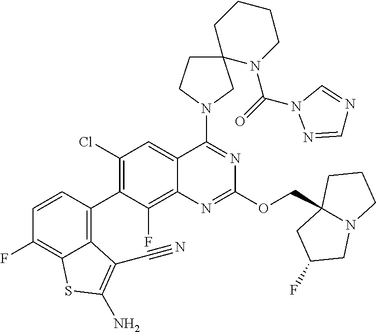 [SMILES: N#Cc1c(N)sc2c(F)ccc(-c3c(Cl)cc4c(N5CCC6(CCCCN6C(=O)n6cncn6)C5)nc(OC[C@@]56CCCN5C[C@H](F)C6)nc4c3F)c12] | 4-(4-(6-(1H-1,2,4-triazole-1-carbonyl)-2,6- diazaspiro[4.5]decan-2-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 763.3 |
| 217 | 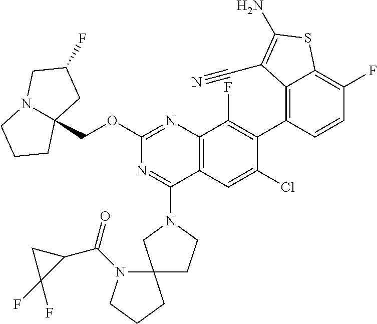 [SMILES: N#Cc1c(N)sc2c(F)ccc(-c3c(Cl)cc4c(N5CCC6(CCCN6C(=O)C6CC6(F)F)C5)nc(OC[C@@]56CCCN5C[C@H](F)C6)nc4c3F)c12] | 2-amino-4-(6-chloro-4-(1-(2,2- difluorocyclopropane-1-carbonyl)-1,7- diazaspiro[4.4]nonan-7-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 758.1 |
| 218 ¹ | 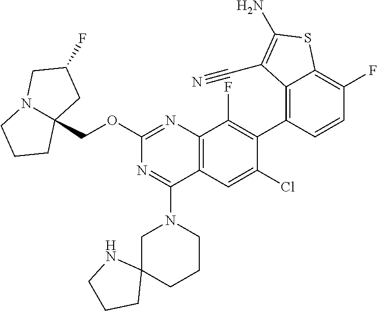 [SMILES: N#Cc1c(N)sc2c(F)ccc(-c3c(Cl)cc4c(N5CCCC6(CCCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1,7-diazaspiro[4.5]decan-7- yl)quinazolin-7-yl)-7-fluorobenzo[b]thiophene- 3-carbonitrile | 668.3 |
| 219 | 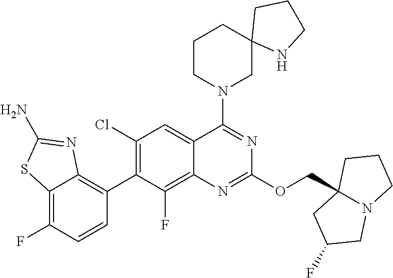 [SMILES: Nc1nc2c(-c3c(Cl)cc4c(N5CCCC6(CCCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,7-diazaspiro[4.5]decan-7- yl)quinazolin-7-yl)-7-fluorobenzo[d]thiazol-2- amine | 644.1 |
| 220 | 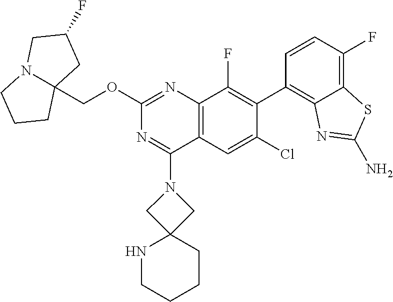 [SMILES: Nc1nc2c(-c3c(Cl)cc4c(N5CC6(CCCCN6)C5)nc(OCC56CCCN5C[C@H](F)C6)nc4c3F)ccc(F)c2s1] | 4-(6-chloro-8-fluoro-2-(((2R)-2- fluorotetrahydro-1H-pyrrolizin-7a(5H)- yl)methoxy)-4-(2,5-diazaspiro[3.5]nonan-2- yl)quinazolin-7-yl)-7-fluorobenzo[d]thiazol-2- amine | 630.12 |
| 221 ² | 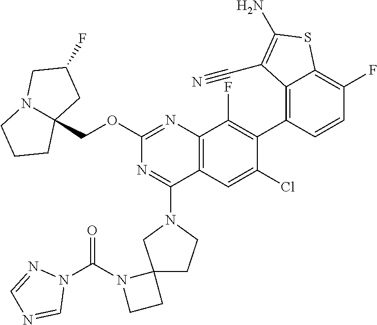 [SMILES: N#Cc1c(N)sc2c(F)ccc(-c3c(Cl)cc4c(N5CCC6(CCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.2 |
| 222 ^{3,4} (3/1 mix) | 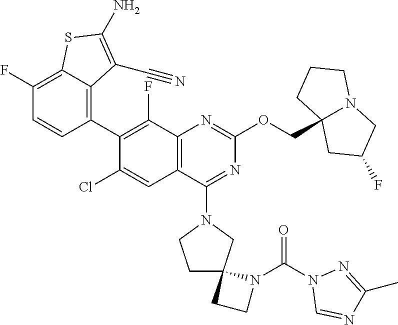 [SMILES: Cc1ncn(C(=O)N2CC[C@]23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-((S)-1-(3-methyl-1H-1,2,4- triazole-1-carbonyl)-1,6-diazaspiro[3.4]octan-6- yl)quinazolin-7-yl)-7-fluorobenzo[b]thiophene- 3-carbonitrile | 749.1 |
| 223 | 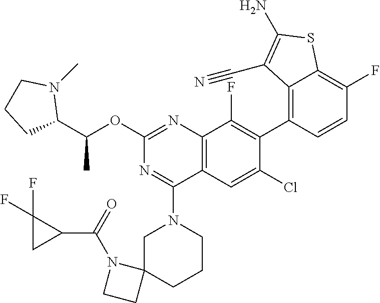 [SMILES: C[C@H](Oc1nc(N2CCCC3(CCN3C(=O)C3CC3(F)F)C2)c2cc(Cl)c(-c3ccc(F)c4sc(N)c(C#N)c34)c(F)c2n1)[C@@H]1CCCN1C] | 2-amino-4-(6-chloro-4-(1-(2,2- difluorocyclopropane-1-carbonyl)-1,6- diazaspiro[3.5]nonan-6-yl)-8-fluoro-2-((S)-1- ((S)-1-methylpyrrolidin-2-yl)ethoxy)quinazolin- 7-yl)-7-fluorobenzo[b]thiophene-3-carbonitrile | 728.2 |
| 224 | 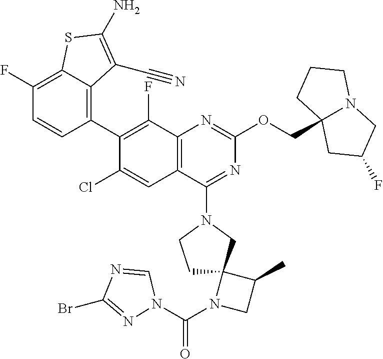 [SMILES: C[C@H]1CN(C(=O)n2cnc(Br)n2)[C@]12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(4-((3S,4R)-1-(3-bromo-1H-1,2,4- triazole-1-carbonyl)-3-methyl-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 828.2 |
| 225 ² | 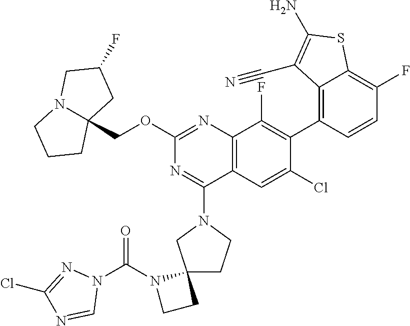 [SMILES: N#Cc1c(N)sc2c(F)ccc(-c3c(Cl)cc4c(N5CC[C@@]6(CCN6C(=O)n6cnc(Cl)n6)C5)nc(OC[C@@]56CCCN5C[C@H](F)C6)nc4c3F)c12] | 2-amino-4-(6-chloro-4-((R)-1-(3-chloro-1H- 1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 770.4 |
| 226 | 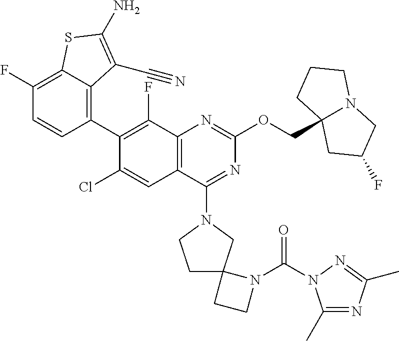 [SMILES: Cc1nc(C)n(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-4-(1-(3,5-dimethyl-1H- 1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 763.5 |
| 227 | 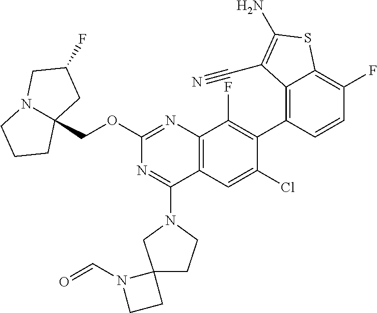 [SMILES: N#Cc1c(N)sc2c(F)ccc(-c3c(Cl)cc4c(N5CCC6(CCN6C=O)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1-formyl-1,6- diazaspiro[3.4]octan-6-yl)quinazolin-7-yl)-7- fluorobenzo[b]thiophene-3-carbonitrile | 668.4 |
| 228 | 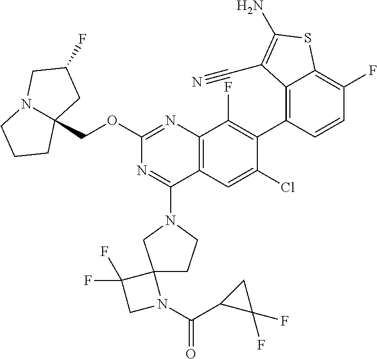 [SMILES: N#Cc1c(N)sc2c(F)ccc(-c3c(Cl)cc4c(N5CCC6(C5)N(C(=O)C5CC5(F)F)CC6(F)F)nc(OC[C@@]56CCCN5C[C@H](F)C6)nc4c3F)c12] | 2-amino-4-(6-chloro-4-(1-(2,2- difluorocyclopropane-1-carbonyl)-3,3-difluoro- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 780.5 |
| 229 ² | 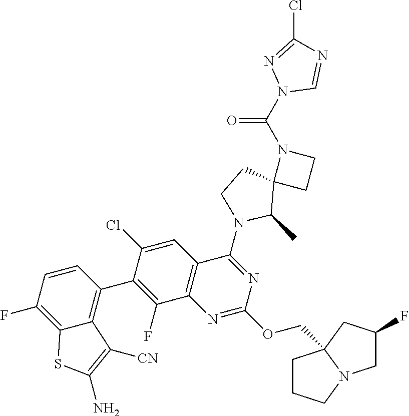 [SMILES: C[C@H]1N(c2nc(OC[C@@]34CCCN3C[C@H](F)C4)nc3c(F)c(-c4ccc(F)c5sc(N)c(C#N)c45)c(Cl)cc23)CC[C@]12CCN2C(=O)n1cnc(Cl)n1] | 2-amino-4-(6-chloro-4-((4S,5R)-1-(3-chloro- 1H-1,2,4-triazole-1-carbonyl)-5-methyl-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 783.4 |
| 230 | 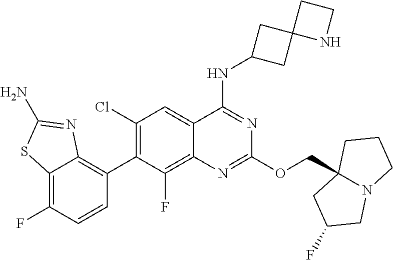 [SMILES: Nc1nc2c(-c3c(Cl)cc4c(NC5CC6(CCN6)C5)nc(OC[C@@]56CCCN5C[C@H](F)C6)nc4c3F)ccc(F)c2s1] | 4-(4-((1-azaspiro[3.3]heptan-6-yl)amino)-6- chloro-8-fluoro-2-(((2R,7aS)-2- fluorotetrahydro-1H-pyrrolizin-7a(5H)- yl)methoxy)quinazolin-7-yl)-7- fluorobenzo[d]thiazol-2-amine | 616.19 |
| 231 ¹ | 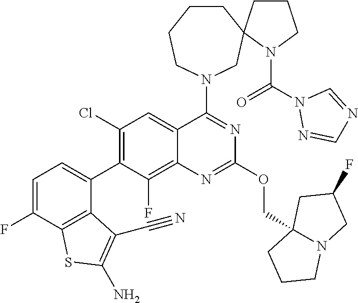 [SMILES: N#Cc1c(N)sc2c(F)ccc(-c3c(Cl)cc4c(N5CCCCC6(CCCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,7- diazaspiro[4.6]undecan-7-yl)-6-chloro-8-fluoro- 2-(((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 777.5 |
| 232 | 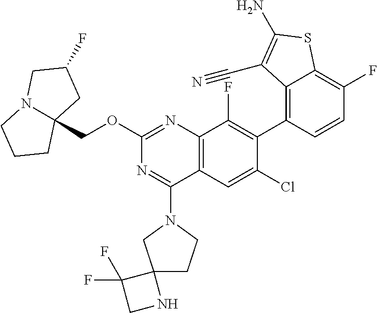 [SMILES: N#Cc1c(N)sc2c(F)ccc(-c3c(Cl)cc4c(N5CCC6(C5)NCC6(F)F)nc(OC[C@@]56CCCN5C[C@H](F)C6)nc4c3F)c12] | 2-amino-4-(6-chloro-4-(3,3-difluoro-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 676.2 |
| 233 | 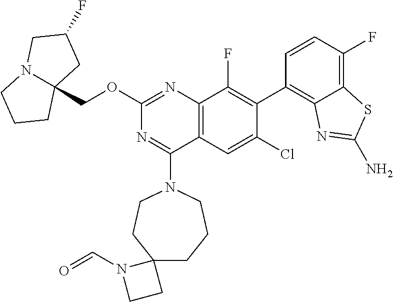 [SMILES: Nc1nc2c(-c3c(Cl)cc4c(N5CCCC6(CC5)CCN6C=O)nc(OC[C@@]56CCCN5C[C@H](F)C6)nc4c3F)ccc(F)c2s1] | 7-(7-(2-amino-7-fluorobenzo[d]thiazol-4-yl)-6- chloro-8-fluoro-2-(((2R,7aS)-2- fluorotetrahydro-1H-pyrrolizin-7a(5H)- yl)methoxy)quinazolin-4-yl)-1,7- diazaspiro[3.6]decane-1-carbaldehyde | 672.2 |
| 234 ² | 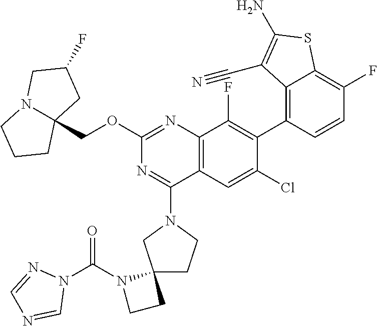 [SMILES: N#Cc1c(N)sc2c(F)ccc(-c3c(Cl)cc4c(N5CC[C@@]6(CCN6C(=O)n6cncn6)C5)nc(OC[C@@]56CCCN5C[C@H](F)C6)nc4c3F)c12] | 4-(4-((R)-1-(1H-1,2,4-triazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.3 |
| 235 | 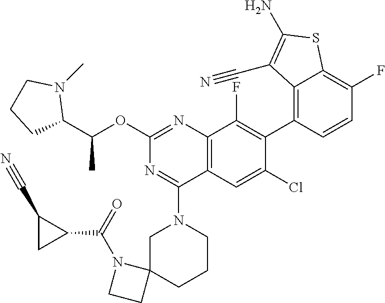 [SMILES: C[C@H](Oc1nc(N2CCCC3(CCN3C(=O)[C@@H]3C[C@H]3C#N)C2)c2cc(Cl)c(-c3ccc(F)c4sc(N)c(C#N)c34)c(F)c2n1)[C@@H]1CCCN1C] | 2-amino-4-(6-chloro-4-(1-((1R,2R)-2- cyanocyclopropane-1-carbonyl)-1,6- diazaspiro[3.5]nonan-6-yl)-8-fluoro-2-((S)-1- ((S)-1-methylpyrrolidin-2-yl)ethoxy)quinazolin- 7-yl)-7-fluorobenzo[b]thiophene-3-carbonitrile | 717.3 |
| 236 ¹ | 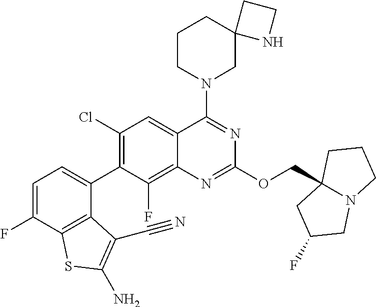 [SMILES: N#Cc1c(N)sc2c(F)ccc(-c3c(Cl)cc4c(N5CCCC6(CCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1,6-diazaspiro[3.5]nonan-6- yl)quinazolin-7-yl)-7-fluorobenzo[b]thiophene- 3-carbonitrile | 654.3 |
| 237 | 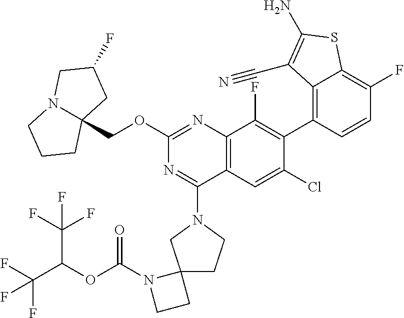 [SMILES: N#Cc1c(N)sc2c(F)ccc(-c3c(Cl)cc4c(N5CCC6(CCN6C(=O)OC(C(F)(F)F)C(F)(F)F)C5)nc(OC[C@@]56CCCN5C[C@H](F)C6)nc4c3F)c12] | 1,1,1,3,3,3-hexafluoropropan-2-yl 6-(7-(2- amino-3-cyano-7-fluorobenzo[b]thiophen-4-yl)- 6-chloro-8-fluoro-2-(((2R,7aS)-2- fluorotetrahydro-1H-pyrrolizin-7a(5H)- yl)methoxy)quinazolin-4-yl)-1,6- diazaspiro[3.4]octane-1-carboxylate | 834.4 |
| 238 | 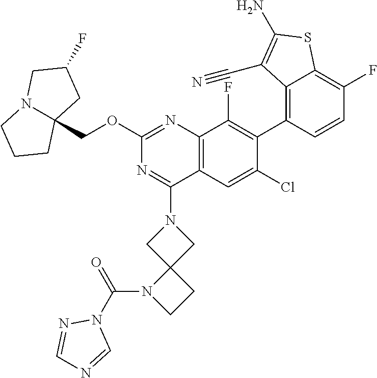 [SMILES: N#Cc1c(N)sc2c(F)ccc(-c3c(Cl)cc4c(N5CC6(CCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,6- diazaspiro[3.3]heptan-6-yl)-6-chloro-8-fluoro- 2-(((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 721.3 |
| 239 | 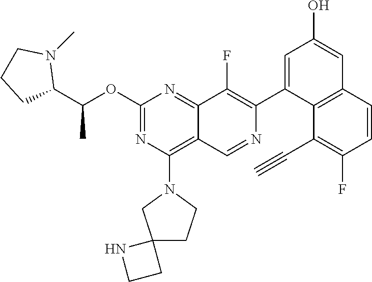 [SMILES: C#Cc1c(F)ccc2cc(O)cc(-c3ncc4c(N5CCC6(CCN6)C5)nc(O[C@@H](C)[C@@H]5CCCN5C)nc4c3F)c12] | 5-ethynyl-6-fluoro-4-(8-fluoro-2-((S)-1-((S)-1- methylpyrrolidin-2-yl)ethoxy)-4-(1,6- diazaspiro[3.4]octan-6-yl)pyrido[4,3- d]pyrimidin-7-yl)naphthalen-2-ol | 571.3 |
| 240 | 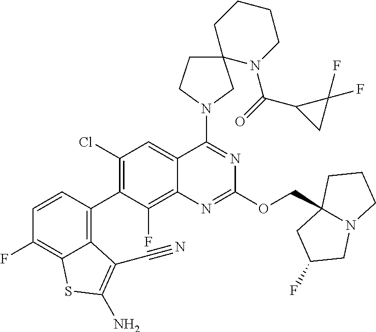 [SMILES: N#Cc1c(N)sc2c(F)ccc(-c3c(Cl)cc4c(N5CCC6(CCCCN6C(=O)C6CC6(F)F)C5)nc(OC[C@@]56CCCN5C[C@H](F)C6)nc4c3F)c12] | 2-amino-4-(6-chloro-4-(6-(2,2- difluorocyclopropane-1-carbonyl)-2,6- diazaspiro[4.5]decan-2-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 772.3 |
| 241 | 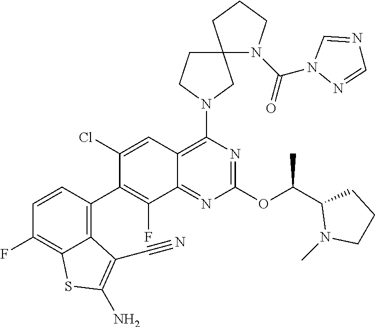 [SMILES: C[C@H](Oc1nc(N2CCC3(CCCN3C(=O)n3cncn3)C2)c2cc(Cl)c(-c3ccc(F)c4sc(N)c(C#N)c34)c(F)c2n1)[C@@H]1CCCN1C] | 4-(4-(1-(1H-1,2,4-triazole-1-carbonyl)-1,7- diazaspiro[4.4]nonan-7-yl)-6-chloro-8-fluoro-2- ((S)-1-((S)-1-methylpyrrolidin-2- yl)ethoxy)quinazolin-7-yl)-2-amino-7- fluorobenzo[b]thiophene-3-carbonitrile | 719.2 |
| 242 | 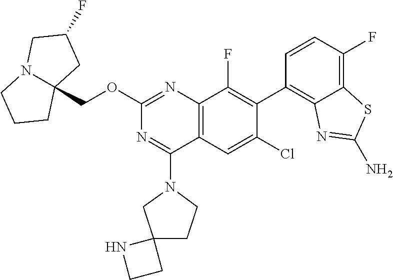 [SMILES: Nc1nc2c(-c3c(Cl)cc4c(N5CCC6(CCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,6-diazaspiro[3.4]octan-6- yl)quinazolin-7-yl)-7-fluorobenzo[d]thiazol-2- amine | 616.1 |
| 243 | 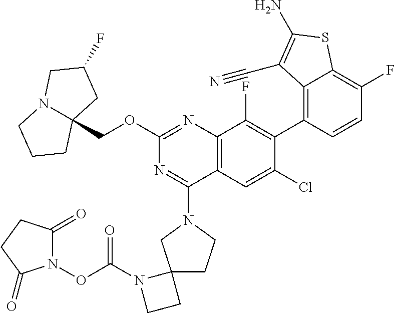 [SMILES: N#Cc1c(N)sc2c(F)ccc(-c3c(Cl)cc4c(N5CCC6(CCN6C(=O)ON6C(=O)CCC6=O)C5)nc(OC[C@@]56CCCN5C[C@H](F)C6)nc4c3F)c12] | 2,5-dioxopyrrolidin-1-yl 6-(7-(2-amino-3- cyano-7-fluorobenzo[b]thiophen-4-yl)-6-chloro- 8-fluoro-2-(((2R,7aS)-2-fluorotetrahydro-1H- pyrrolizin-7a(5H)-yl)methoxy)quinazolin-4-yl)- 1,6-diazaspiro[3.4]octane-1-carboxylate | 781.1 |
| 244 | 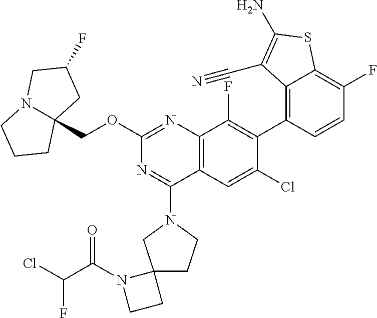 [SMILES: N#Cc1c(N)sc2c(F)ccc(-c3c(Cl)cc4c(N5CCC6(CCN6C(=O)C(F)Cl)C5)nc(OC[C@@]56CCCN5C[C@H](F)C6)nc4c3F)c12] | 2-amino-4-(6-chloro-4-(1-(2-chloro-2- fluoroacetyl)-1,6-diazaspiro[3.4]octan-6-yl)-8- fluoro-2-(((2R,7aS)-2-fluorotetrahydro-1H- pyrrolizin-7a(5H)-yl)methoxy)quinazolin-7-yl)- 7-fluorobenzo[b]thiophene-3-carbonitrile | 735.2 |
| 245 | 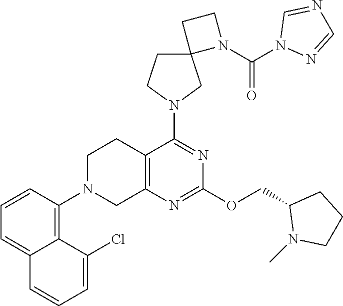 [SMILES: CN1CCC[C@H]1COc1nc2c(c(N3CCC4(CCN4C(=O)n4cncn4)C3)n1)CCN(c1cccc3cccc(Cl)c13)C2] | (6-(7-(8-chloronaphthalen-1-yl)-2-(((S)-1- methylpyrrolidin-2-yl)methoxy)-5,6,7,8- tetrahydropyrido[3,4-d]pyrimidin-4-yl)-1,6- diazaspiro[3.4]octan-1-yl)(1H-1,2,4-triazol-1- yl)methanone | 614.2 |
| 246 ¹ | 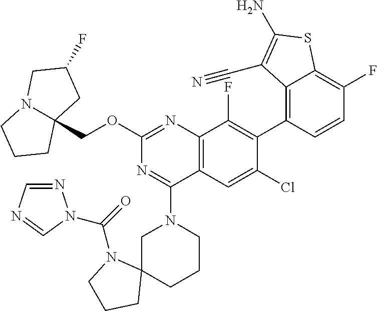 [SMILES: N#Cc1c(N)sc2c(F)ccc(-c3c(Cl)cc4c(N5CCCC6(CCCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,7- diazaspiro[4.5]decan-7-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 763.4 |
| 247 ³ | 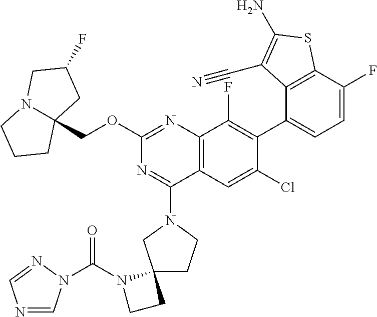 [SMILES: N#Cc1c(N)sc2c(F)ccc(-c3c(Cl)cc4c(N5CC[C@@]6(CCN6C(=O)n6cncn6)C5)nc(OC[C@@]56CCCN5C[C@H](F)C6)nc4c3F)c12] | 4-(4-((R)-1-(1H-1,2,4-triazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.3 |
| 248 ⁴ (1/4 mix) | 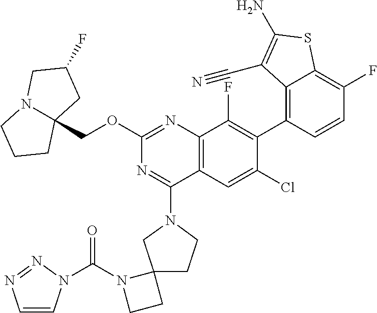 [SMILES: N#Cc1c(N)sc2c(F)ccc(-c3c(Cl)cc4c(N5CCC6(CCN6C(=O)n6ccnn6)C5)nc(OC[C@@]56CCCN5C[C@H](F)C6)nc4c3F)c12] | 4-(4-(1-(1H-1,2,3-triazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.1 |
| 249 | 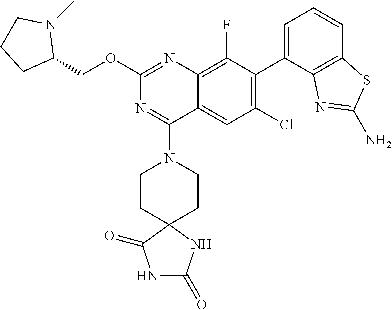 [SMILES: CN1CCC[C@H]1COc1nc(N2CCC3(CC2)NC(=O)NC3=O)c2cc(Cl)c(-c3cccc4sc(N)nc34)c(F)c2n1] | 8-(7-(2-aminobenzo[d]thiazol-4-yl)-6-chloro-8- fluoro-2-(((S)-1-methylpyrrolidin-2- yl)methoxy)quinazolin-4-yl)-1,3,8- triazaspiro[4.5]decane-2,4-dione | 611.1 |
| 250 ^{2,4} (3/1 mix) | 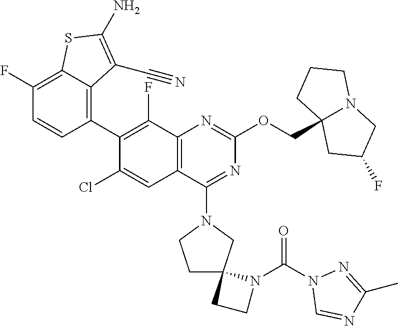 [SMILES: Cc1ncn(C(=O)N2CC[C@]23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-((S)-1-(3-methyl-1H-1,2,4- triazole-1-carbonyl)-1,6-diazaspiro[3.4]octan-6- yl)quinazolin-7-yl)-7-fluorobenzo[b]thiophene- 3-carbonitrile | 749.2 |
| 251 | 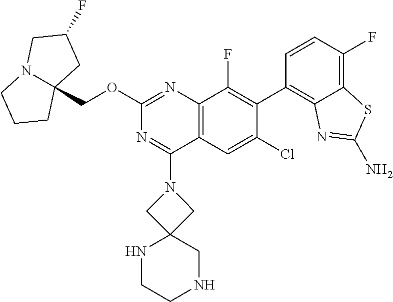 [SMILES: Nc1nc2c(-c3c(Cl)cc4c(N5CC6(CNCCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(2,5,8-triazaspiro[3.5]nonan-2- yl)quinazolin-7-yl)-7-fluorobenzo[d]thiazol-2- amine | 631.31 |
| 252 ¹ | 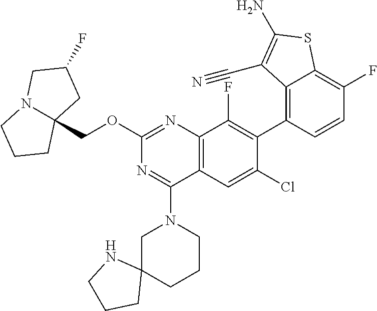 [SMILES: N#Cc1c(N)sc2c(F)ccc(-c3c(Cl)cc4c(N5CCCC6(CCCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1,7-diazaspiro[4.5]decan-7- yl)quinazolin-7-yl)-7-fluorobenzo[b]thiophene- 3-carbonitrile | 668.3 |
| 253 | 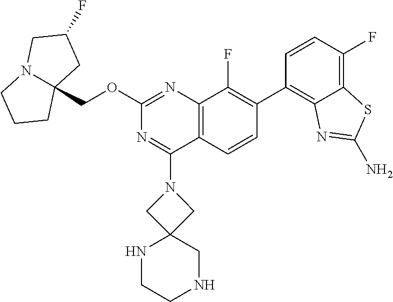 [SMILES: Nc1nc2c(-c3ccc4c(N5CC6(CNCCN6)C5)nc(OC[C@@]56CCCN5C[C@H](F)C6)nc4c3F)ccc(F)c2s1] | 7-fluoro-4-(8-fluoro-2-(((2R,7aS)-2- fluorotetrahydro-1H-pyrrolizin-7a(5H)- yl)methoxy)-4-(2,5,8-triazaspiro[3.5]nonan-2- yl)quinazolin-7-yl)benzo[d]thiazol-2-amine | 597.1 |
| 254 | 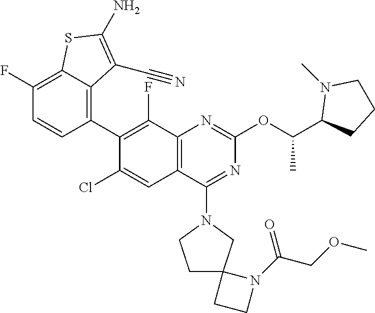 [SMILES: COCC(=O)N1CCC12CCN(c1nc(O[C@@H](C)[C@@H]3CCCN3C)nc3c(F)c(-c4ccc(F)c5sc(N)c(C#N)c45)c(Cl)cc13)C2] | 2-amino-4-(6-chloro-8-fluoro-4-(1-(2- methoxyacetyl)-1,6-diazaspiro[3.4]octan-6-yl)- 2-((S)-1-((S)-1-methylpyrrolidin-2- yl)ethoxy)quinazolin-7-yl)-7- fluorobenzo[b]thiophene-3-carbonitrile | 682.1 |
| 255 ² | 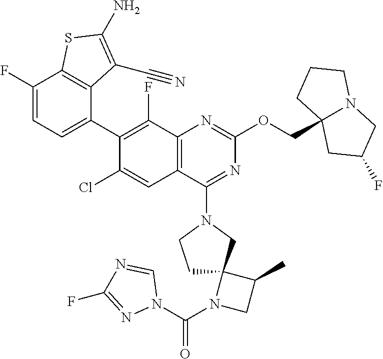 [SMILES: C[C@H]1CN(C(=O)n2cnc(F)n2)[C@]12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-4-((3S,4R)-1-(3- fluoro-1H-1,2,4-triazole-1-carbonyl)-3-methyl- 1,6-diazaspiro[3.4]octan-6-yl)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 767.4 |
| 256 | 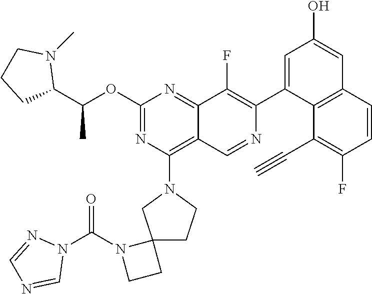 [SMILES: C#Cc1c(F)ccc2cc(O)cc(-c3ncc4c(N5CCC6(CCN6C(=O)n6cncn6)C5)nc(O[C@@H](C)[C@@H]5CCCN5C)nc4c3F)c12] | (6-(7-(8-ethynyl-7-fluoro-3-hydroxynaphthalen- 1-yl)-8-fluoro-2-((S)-1-((S)-1-methylpyrrolidin- 2-yl)ethoxy)pyrido[4,3-d]pyrimidin-4-yl)-1,6- diazaspiro[3.4]octan-1-yl)(1H-1,2,4-triazol-1- yl)methanone | 666.5 |
| 257 | 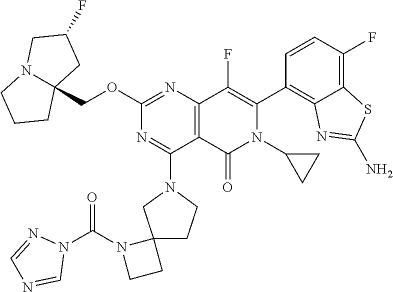 [SMILES: Nc1nc2c(-c3c(F)c4nc(OC[C@@]56CCCN5C[C@H](F)C6)nc(N5CCC6(CCN6C(=O)n6cncn6)C5)c4c(=O)n3C3CC3)ccc(F)c2s1] | 4-(1-(1H-1,2,4-triazole-1-carbonyl)-1,6- diazaspiro[3.4]octan-6-yl)-7-(2-amino-7- fluorobenzo[d]thiazol-4-yl)-6-cyclopropyl-8- fluoro-2-(((2R,7aS)-2-fluorotetrahydro-1H- pyrrolizin-7a(5H)-yl)methoxy)pyrido[4,3- d]pyrimidin-5(6H)-one | 734.3 |
| 258 | 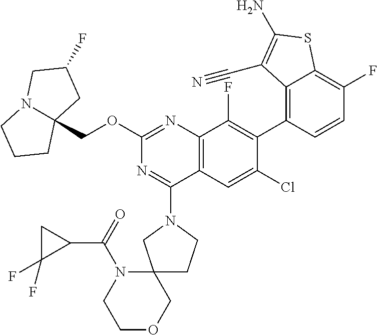 [SMILES: N#Cc1c(N)sc2c(F)ccc(-c3c(Cl)cc4c(N5CCC6(COCCN6C(=O)C6CC6(F)F)C5)nc(OC[C@@]56CCCN5C[C@H](F)C6)nc4c3F)c12] | 2-amino-4-(6-chloro-4-(6-(2,2- difluorocyclopropane-1-carbonyl)-9-oxa-2,6- diazaspiro[4.5]decan-2-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 774.4 |
| 259 | 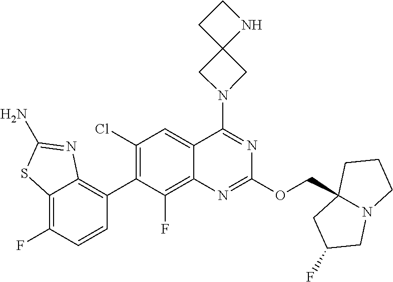 [SMILES: Nc1nc2c(-c3c(Cl)cc4c(N5CC6(CCN6)C5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,6-diazaspiro[3.3]heptan-6- yl)quinazolin-7-yl)-7-fluorobenzo[d]thiazol-2- amine | 602.4 |
| 260 ¹ | 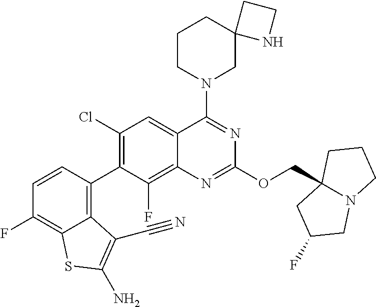 [SMILES: N#Cc1c(N)sc2c(F)ccc(-c3c(Cl)cc4c(N5CCCC6(CCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1,6-diazaspiro[3.5]nonan-6- yl)quinazolin-7-yl)-7-fluorobenzo[b]thiophene- 3-carbonitrile | 654.1 |
| 261 | 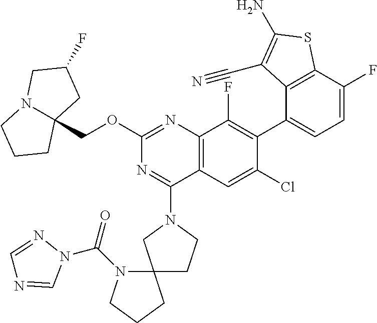 [SMILES: N#Cc1c(N)sc2c(F)ccc(-c3c(Cl)cc4c(N5CCC6(CCCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,7- diazaspiro[4.4]nonan-7-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 749.1 |
| 262 | 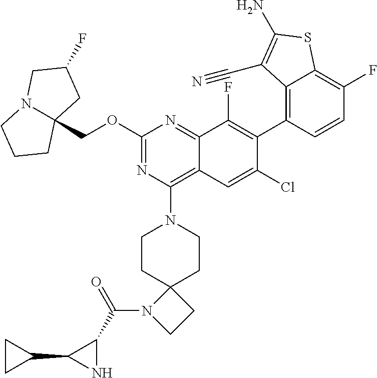 [SMILES: N#Cc1c(N)sc2c(F)ccc(-c3c(Cl)cc4c(N5CCC6(CC5)CCN6C(=O)[C@@H]5N[C@H]5C5CC5)nc(OC[C@@]56CCCN5C[C@H](F)C6)nc4c3F)c12] | 2-amino-4-(6-chloro-4-(1-((2R,3S)-3- cyclopropylaziridine-2-carbonyl)-1,7- diazaspiro[3.5]nonan-7-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 763.4 |
| 263 ² | 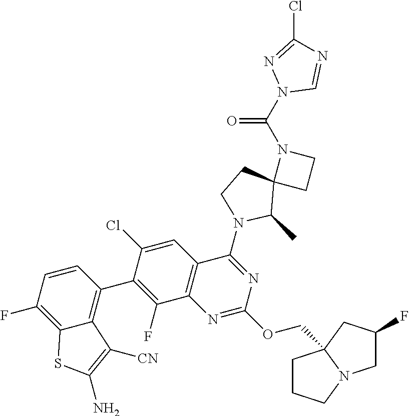 [SMILES: C[C@H]1N(c2nc(OC[C@@]34CCCN3C[C@H](F)C4)nc3c(F)c(-c4ccc(F)c5sc(N)c(C#N)c45)c(Cl)cc23)CC[C@@]12CCN2C(=O)n1cnc(Cl)n1] | 2-amino-4-(6-chloro-4-((4R,5R)-1-(3-chloro- 1H-1,2,4-triazole-1-carbonyl)-5-methyl-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 783.4 |
| 264 | 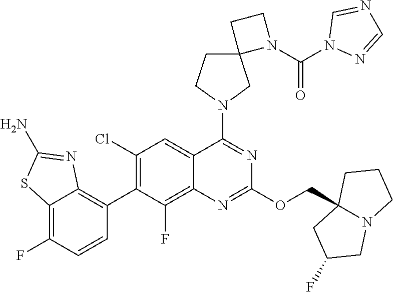 [SMILES: Nc1nc2c(-c3c(Cl)cc4c(N5CCC6(CCN6C(=O)n6cncn6)C5)nc(OC[C@@]56CCCN5C[C@H](F)C6)nc4c3F)ccc(F)c2s1] | (6-(7-(2-amino-7-fluorobenzo[d]thiazol-4-yl)-6- chloro-8-fluoro-2-(((2R,7aS)-2- fluorotetrahydro-1H-pyrrolizin-7a(5H)- yl)methoxy)quinazolin-4-yl)-1,6- diazaspiro[3.4]octan-1-yl)(1H-1,2,4-triazol-1- yl)methanone | 711.5 |
| 265 | 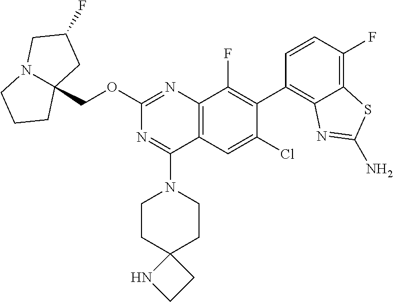 [SMILES: Nc1nc2c(-c3c(Cl)cc4c(N5CCC6(CCN6)CC5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,7-diazaspiro[3.5]nonan-7- yl)quinazolin-7-yl)-7-fluorobenzo[d]thiazol-2- amine | 630.2 |
| 266 ⁵ | 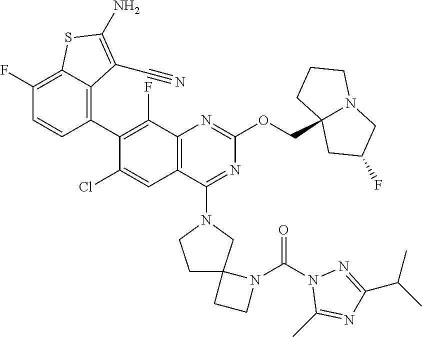 [SMILES: Cc1nc(C(C)C)nn1C(=O)N1CCC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(1-(3-isopropyl-5-methyl-1H- 1,2,4-triazole-1-carbonyl)-1,6- diazaspiro[3.4]octan-6-yl)quinazolin-7-yl)-7- fluorobenzo[b]thiophene-3-carbonitrile | 791.7 |
| 267 | 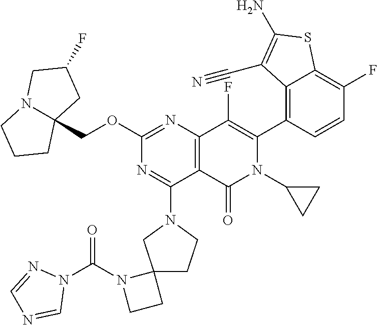 [SMILES: N#Cc1c(N)sc2c(F)ccc(-c3c(F)c4nc(OC[C@@]56CCCN5C[C@H](F)C6)nc(N5CCC6(CCN6C(=O)n6cncn6)C5)c4c(=O)n3C3CC3)c12] | 4-(4-(1-(1H-1,2,4-triazole-1-carbonyl)-1,6- diazaspiro[3.4]octan-6-yl)-6-cyclopropyl-8- fluoro-2-(((2R,7aS)-2-fluorotetrahydro-1H- pyrrolizin-7a(5H)-yl)methoxy)-5-oxo-5,6- dihydropyrido[4,3-d]pyrimidin-7-yl)-2-amino- 7-fluorobenzo[b]thiophene-3-carbonitrile | 760.2 |
| 268 | 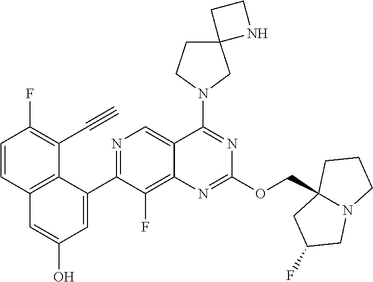 [SMILES: C#Cc1c(F)ccc2cc(O)cc(-c3ncc4c(N5CCC6(CCN6)C5)nc(OC[C@@]56CCCN5C[C@H](F)C6)nc4c3F)c12] | 5-ethynyl-6-fluoro-4-(8-fluoro-2-(((2R,7aS)-2- fluorotetrahydro-1H-pyrrolizin-7a(5H)- yl)methoxy)-4-(1,6-diazaspiro[3.4]octan-6- yl)pyrido[4,3-d]pyrimidin-7-yl)naphthalen-2-ol | 601.2 |
| 269 | 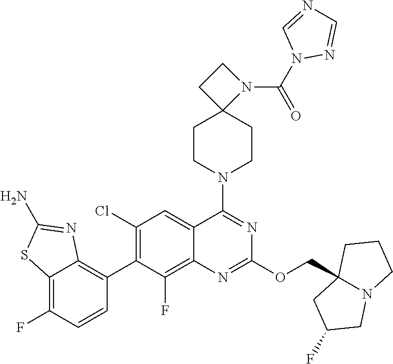 [SMILES: Nc1nc2c(-c3c(Cl)cc4c(N5CCC6(CC5)CCN6C(=O)n5cncn5)nc(OC[C@@]56CCCN5C[C@H](F)C6)nc4c3F)ccc(F)c2s1] | (7-(7-(2-amino-7-fluorobenzo[d]thiazol-4-yl)-6- chloro-8-fluoro-2-(((2R,7aS)-2- fluorotetrahydro-1H-pyrrolizin-7a(5H)- yl)methoxy)quinazolin-4-yl)-1,7- diazaspiro[3.5]nonan-1-yl)(1H-1,2,4-triazol-1- yl)methanone | 725.2 |
| 270 | 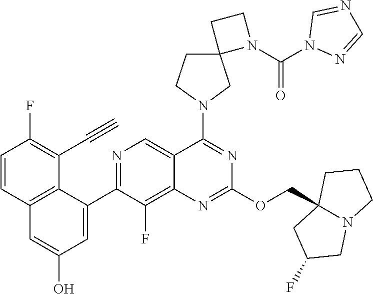 [SMILES: C#Cc1c(F)ccc2cc(O)cc(-c3ncc4c(N5CCC6(CCN6C(=O)n6cncn6)C5)nc(OC[C@@]56CCCN5C[C@H](F)C6)nc4c3F)c12] | (6-(7-(8-ethynyl-7-fluoro-3-hydroxynaphthalen- 1-yl)-8-fluoro-2-(((2R,7aS)-2-fluorotetrahydro- 1H-pyrrolizin-7a(5H)-yl)methoxy)pyrido[4,3- d]pyrimidin-4-yl)-1,6-diazaspiro[3.4]octan-1- yl)(1H-1,2,4-triazol-1-yl)methanone | 696.1 |
| 271 | 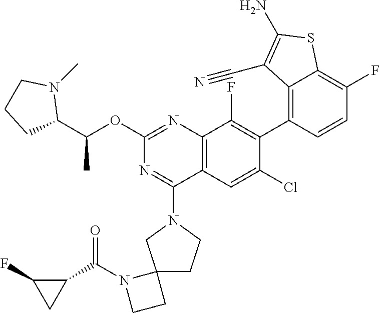 [SMILES: C[C@H](Oc1nc(N2CCC3(CCN3C(=O)[C@@H]3C[C@H]3F)C2)c2cc(Cl)c(-c3ccc(F)c4sc(N)c(C#N)c34)c(F)c2n1)[C@@H]1CCCN1C] | 2-amino-4-(6-chloro-8-fluoro-4-(1-((1S,2R)-2- fluorocyclopropane-1-carbonyl)-1,6- diazaspiro[3.4]octan-6-yl)-2-((S)-1-((S)-1- methylpyrrolidin-2-yl)ethoxy)quinazolin-7-yl)- 7-fluorobenzo[b]thiophene-3-carbonitrile | 696.4 |
| 272 | 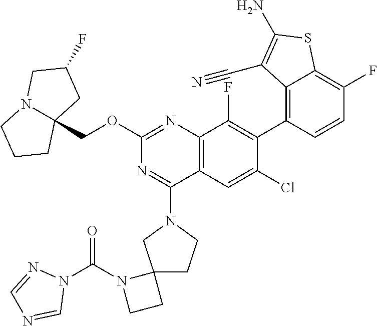 [SMILES: N#Cc1c(N)sc2c(F)ccc(-c3c(Cl)cc4c(N5CCC6(CCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.2 |
| 273 | 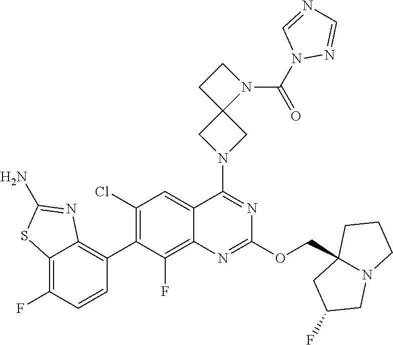 [SMILES: Nc1nc2c(-c3c(Cl)cc4c(N5CC6(CCN6C(=O)n6cncn6)C5)nc(OC[C@@]56CCCN5C[C@H](F)C6)nc4c3F)ccc(F)c2s1] | (6-(7-(2-amino-7-fluorobenzo[d]thiazol-4-yl)-6- chloro-8-fluoro-2-(((2R,7aS)-2- fluorotetrahydro-1H-pyrrolizin-7a(5H)- yl)methoxy)quinazolin-4-yl)-1,6- diazaspiro[3.3]heptan-1-yl)(1H-1,2,4-triazol-1- yl)methanone | 697.2 |
| 274 | 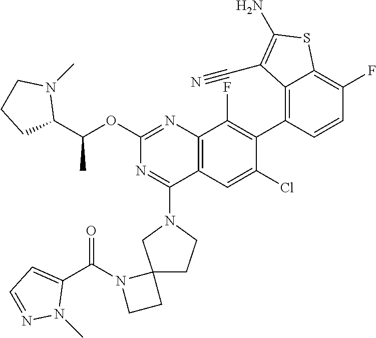 [SMILES: C[C@H](Oc1nc(N2CCC3(CCN3C(=O)c3ccnn3C)C2)c2cc(Cl)c(-c3ccc(F)c4sc(N)c(C#N)c34)c(F)c2n1)[C@@H]1CCCN1C] | 2-amino-4-(6-chloro-8-fluoro-4-(1-(1-methyl- 1H-pyrazole-5-carbonyl)-1,6- diazaspiro[3.4]octan-6-yl)-2-((S)-1-((S)-1- methylpyrrolidin-2-yl)ethoxy)quinazolin-7-yl)- 7-fluorobenzo[b]thiophene-3-carbonitrile | 718.4 |
| 275 | 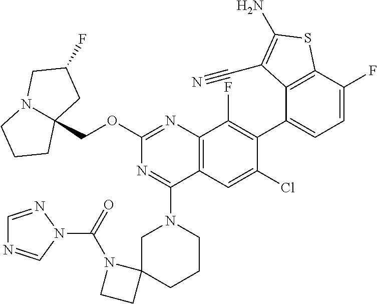 [SMILES: N#Cc1c(N)sc2c(F)ccc(-c3c(Cl)cc4c(N5CCCC6(CCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,6- diazaspiro[3.5]nonan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 749.2 |
| 276 | 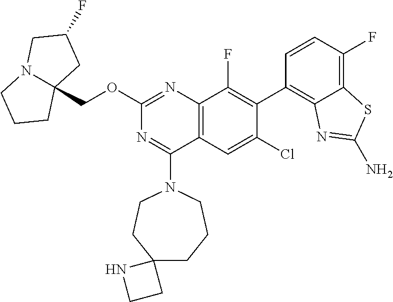 [SMILES: Nc1nc2c(-c3c(Cl)cc4c(N5CCCC6(CCN6)CC5)nc(OC[C@@]56CCCN5C[C@H](F)C6)nc4c3F)ccc(F)c2s1] | 4-(6-chloro-8-fluoro-2-(((2R,7aS)-2- fluorotetrahydro-1H-pyrrolizin-7a(5H)- yl)methoxy)-4-(1,7-diazaspiro[3.6]decan-7- yl)quinazolin-7-yl)-7-fluorobenzo[d]thiazol-2- amine | 644.4 |
| 277 | 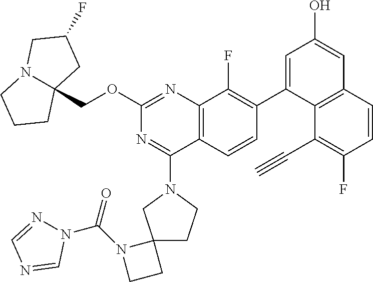 [SMILES: C#Cc1c(F)ccc2cc(O)cc(-c3ccc4c(N5CCC6(CCN6C(=O)n6cncn6)C5)nc(OC[C@@]56CCCN5C[C@H](F)C6)nc4c3F)c12] | (6-(7-(8-ethynyl-7-fluoro-3-hydroxynaphthalen- 1-yl)-8-fluoro-2-(((2R,7aS)-2-fluorotetrahydro- 1H-pyrrolizin-7a(5H)-yl)methoxy)quinazolin-4- yl)-1,6-diazaspiro[3.4]octan-1-yl)(1H-1,2,4- triazol-1-yl)methanone | 695.3 |
| 278 ² | 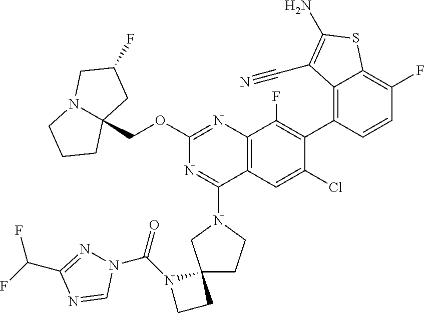 [SMILES: N#Cc1c(N)sc2c(F)ccc(-c3c(Cl)cc4c(N5CC[C@@]6(CCN6C(=O)n6cnc(C(F)F)n6)C5)nc(OC[C@@]56CCCN5C[C@H](F)C6)nc4c3F)c12] | 2-amino-4-(6-chloro-4-((R)-1-(3- (difluoromethyl)-1H-1,2,4-triazole-1-carbonyl)- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 785.2 |
| 279 | 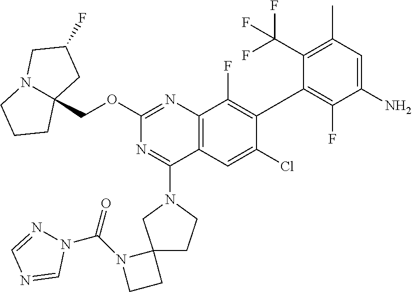 [SMILES: Cc1cc(N)c(F)c(-c2c(Cl)cc3c(N4CCC5(CCN5C(=O)n5cncn5)C4)nc(OC[C@@]45CCCN4C[C@H](F)C5)nc3c2F)c1C(F)(F)F] | (6-(7-(3-amino-2-fluoro-5-methyl-6- (trifluoromethyl)phenyl)-6-chloro-8-fluoro-2- (((2R,7aS)-2-fluorotetrahydro-1H-pyrrolizin- 7a(5H)-yl)methoxy)quinazolin-4-yl)-1,6- diazaspiro[3.4]octan-1-yl)(1H-1,2,4-triazol-1- yl)methanone | 736.3 |
| 280 | 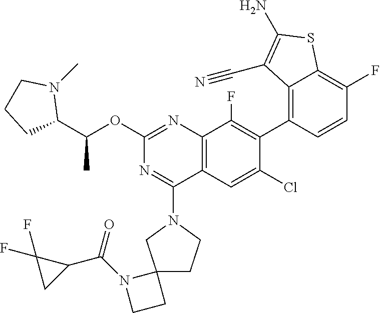 [SMILES: C[C@H](Oc1nc(N2CCC3(CCN3C(=O)C3CC3(F)F)C2)c2cc(Cl)c(-c3ccc(F)c4sc(N)c(C#N)c34)c(F)c2n1)[C@@H]1CCCN1C] | 2-amino-4-(6-chloro-4-(1-(2,2- difluorocyclopropane-1-carbonyl)-1,6- diazaspiro[3.4]octan-6-yl)-8-fluoro-2-((S)-1- ((S)-1-methylpyrrolidin-2-yl)ethoxy)quinazolin- 7-yl)-7-fluorobenzo[b]thiophene-3-carbonitrile | 714.2 |
| 281 ² | 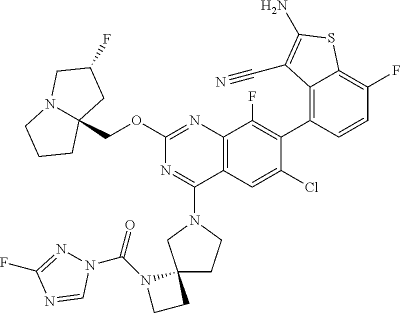 [SMILES: N#Cc1c(N)sc2c(F)ccc(-c3c(Cl)cc4c(N5CC[C@@]6(CCN6C(=O)n6cnc(F)n6)C5)nc(OC[C@@]56CCCN5C[C@H](F)C6)nc4c3F)c12] | 2-amino-4-(6-chloro-8-fluoro-4-((R)-1-(3- fluoro-1H-1,2,4-triazole-1-carbonyl)-1,6- diazaspiro[3.4]octan-6-yl)-2-(((2R,7aS)-2- fluorotetrahydro-1H-pyrrolizin-7a(5H)- yl)methoxy)quinazolin-7-yl)-7- fluorobenzo[b]thiophene-3-carbonitrile | 753.1 |
| 282 | 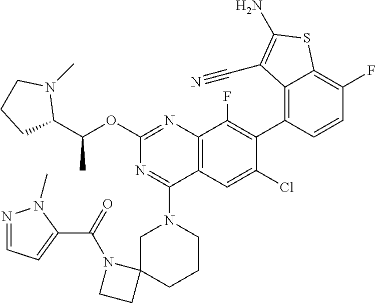 [SMILES: C[C@H](Oc1nc(N2CCCC3(CCN3C(=O)c3ccnn3C)C2)c2cc(Cl)c(-c3ccc(F)c4sc(N)c(C#N)c34)c(F)c2n1)[C@@H]1CCCN1C] | 2-amino-4-(6-chloro-8-fluoro-4-(1-(1-methyl- 1H-pyrazole-5-carbonyl)-1,6- diazaspiro[3.5]nonan-6-yl)-2-((S)-1-((S)-1- methylpyrrolidin-2-yl)ethoxy)quinazolin-7-yl)- 7-fluorobenzo[b]thiophene-3-carbonitrile | 732.24 |
| 283 | 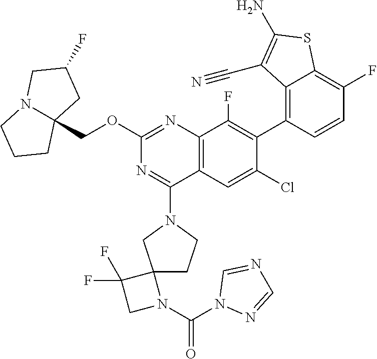 [SMILES: N#Cc1c(N)sc2c(F)ccc(-c3c(Cl)cc4c(N5CCC6(C5)N(C(=O)n5cncn5)CC6(F)F)nc(OC[C@@]56CCCN5C[C@H](F)C6)nc4c3F)c12] | 2-amino-4-(6-chloro-4-(3,3-difluoro-1-(1H- 1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 771.2 |
| 284 | 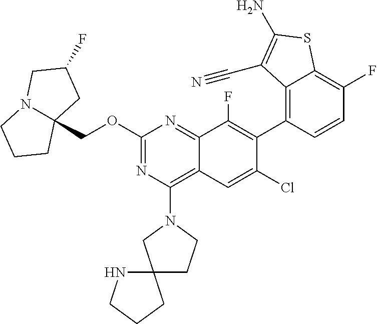 [SMILES: N#Cc1c(N)sc2c(F)ccc(-c3c(Cl)cc4c(N5CCC6(CCCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1,7-diazaspiro[4.4]nonan-7- yl)quinazolin-7-yl)-7-fluorobenzo[b]thiophene- 3-carbonitrile | 654.5 |
| 285 ² | 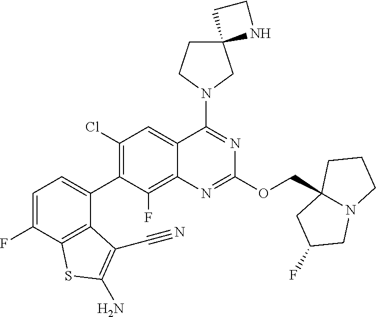 [SMILES: N#Cc1c(N)sc2c(F)ccc(-c3c(Cl)cc4c(N5CC[C@]6(CCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-((R)-1,6-diazaspiro[3.4]octan-6- yl)quinazolin-7-yl)-7-fluorobenzo[b]thiophene- 3-carbonitrile | 640.3 |
| 286 | 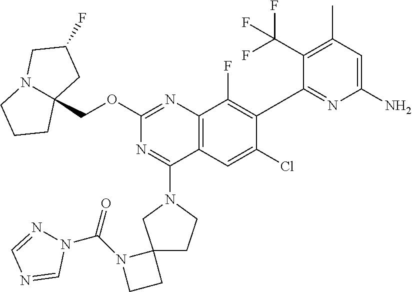 [SMILES: Cc1cc(N)nc(-c2c(Cl)cc3c(N4CCC5(CCN5C(=O)n5cncn5)C4)nc(OC[C@@]45CCCN4C[C@H](F)C5)nc3c2F)c1C(F)(F)F] | (6-(7-(6-amino-4-methyl-3- (trifluoromethyl)pyridin-2-yl)-6-chloro-8- fluoro-2-(((2R,7aS)-2-fluorotetrahydro-1H- pyrrolizin-7a(5H)-yl)methoxy)quinazolin-4-yl)- 1,6-diazaspiro[3.4]octan-1-yl)(1H-1,2,4-triazol- 1-yl)methanone | 719.4 |
| 287 | 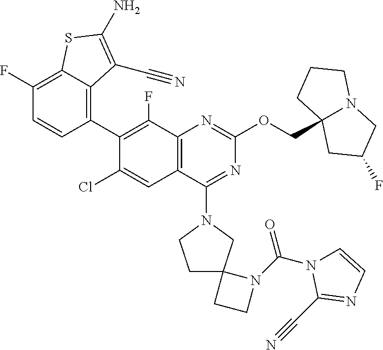 [SMILES: N#Cc1c(N)sc2c(F)ccc(-c3c(Cl)cc4c(N5CCC6(CCN6C(=O)n6ccnc6C#N)C5)nc(OC[C@@]56CCCN5C[C@H](F)C6)nc4c3F)c12] | 1-(6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonyl)-1H- imidazole-2-carbonitrile | 759.4 |
| 288 ⁵ | 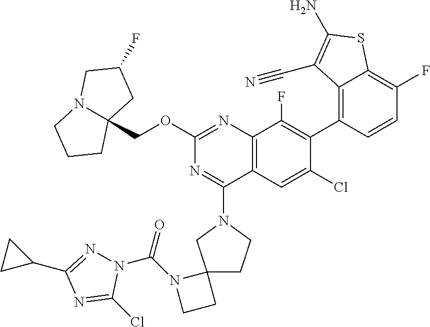 [SMILES: N#Cc1c(N)sc2c(F)ccc(-c3c(Cl)cc4c(N5CCC6(CCN6C(=O)n6nc(C7CC7)nc6Cl)C5)nc(OC[C@@]56CCCN5C[C@H](F)C6)nc4c3F)c12] | 2-amino-4-(6-chloro-4-(1-(5-chloro-3- cyclopropyl-1H-1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 809.5 |
| 289 | 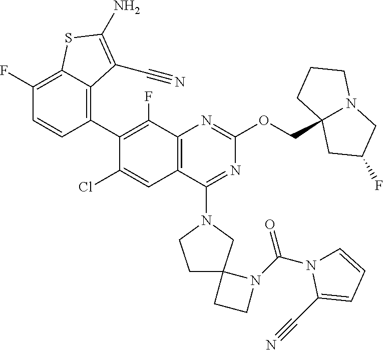 [SMILES: N#Cc1c(N)sc2c(F)ccc(-c3c(Cl)cc4c(N5CCC6(CCN6C(=O)n6cccc6C#N)C5)nc(OC[C@@]56CCCN5C[C@H](F)C6)nc4c3F)c12] | 1-(6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonyl)-1H-pyrrole- 2-carbonitrile | 758.2 |
| 290 ⁵ | 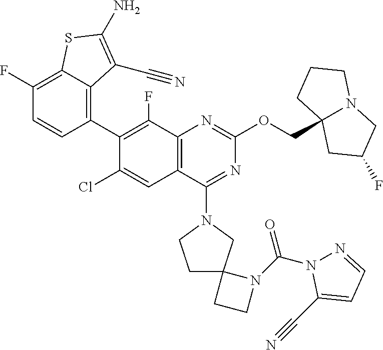 [SMILES: N#Cc1c(N)sc2c(F)ccc(-c3c(Cl)cc4c(N5CCC6(CCN6C(=O)n6nccc6C#N)C5)nc(OC[C@@]56CCCN5C[C@H](F)C6)nc4c3F)c12] | 1-(6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonyl)-1H-pyrazole- 5-carbonitrile | 759.2 |
| 291 ² | 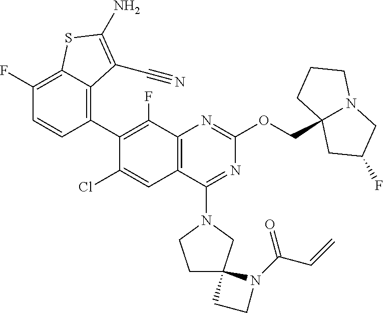 [SMILES: C=CC(=O)N1CC[C@@]12CCN(c1nc(OC[C@@]34CCCN3C[C@H](F)C4)nc3c(F)c(-c4ccc(F)c5sc(N)c(C#N)c45)c(Cl)cc13)C2] | 4-(4-((R)-1-acryloyl-1,6-diazaspiro[3.4]octan-6- yl)-6-chloro-8-fluoro-2-(((2R,7aS)-2- fluorotetrahydro-1H-pyrrolizin-7a(5H)- yl)methoxy)quinazolin-7-yl)-2-amino-7- fluorobenzo[b]thiophene-3-carbonitrile | 694.2 |
| 292 | 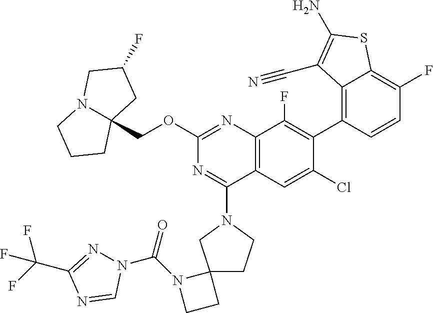 [SMILES: N#Cc1c(N)sc2c(F)ccc(-c3c(Cl)cc4c(N5CCC6(CCN6C(=O)n6cnc(C(F)(F)F)n6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1-(3-(trifluoromethyl)-1H-1,2,4- triazole-1-carbonyl)-1,6-diazaspiro[3.4]octan-6- yl)quinazolin-7-yl)-7-fluorobenzo[b]thiophene- 3-carbonitrile | 803.4 |
| 293 ⁵ | 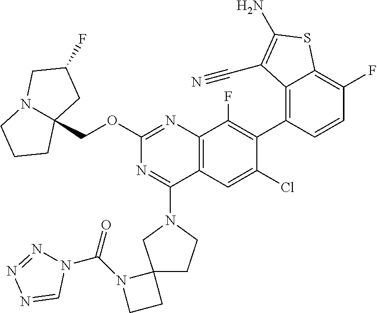 [SMILES: N#Cc1c(N)sc2c(F)ccc(-c3c(Cl)cc4c(N5CCC6(CCN6C(=O)n6cnnn6)C5)nc(OC[C@@]56CCCN5C[C@H](F)C6)nc4c3F)c12] | 4-(4-(1-(1H-tetrazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 736.2 |
| 294 | 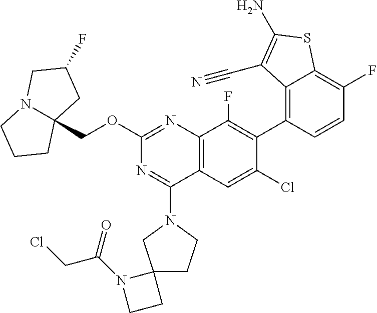 [SMILES: N#Cc1c(N)sc2c(F)ccc(-c3c(Cl)cc4c(N5CCC6(CCN6C(=O)CCl)C5)nc(OC[C@@]56CCCN5C[C@H](F)C6)nc4c3F)c12] | 2-amino-4-(6-chloro-4-(1-(2-chloroacetyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 716.4 |
| 295 | 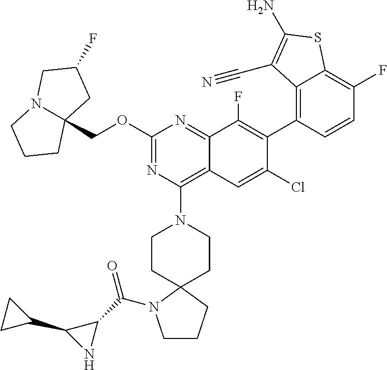 [SMILES: N#Cc1c(N)sc2c(F)ccc(-c3c(Cl)cc4c(N5CCC6(CCCN6C(=O)[C@@H]6N[C@H]6C6CC6)CC5)nc(OC[C@@]56CCCN5C[C@H](F)C6)nc4c3F)c12] | 2-amino-4-(6-chloro-4-(1-((2R,3S)-3- cyclopropylaziridine-2-carbonyl)-1,8- diazaspiro[4.5]decan-8-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 777.4 |
| 296 | 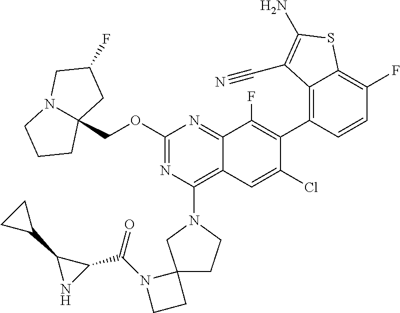 [SMILES: N#Cc1c(N)sc2c(F)ccc(-c3c(Cl)cc4c(N5CCC6(CCN6C(=O)[C@@H]6N[C@H]6C6CC6)C5)nc(OC[C@@]56CCCN5C[C@H](F)C6)nc4c3F)c12] | 2-amino-4-(6-chloro-4-(1-((2R,3S)-3- cyclopropylaziridine-2-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 749.7 |
| 297 | 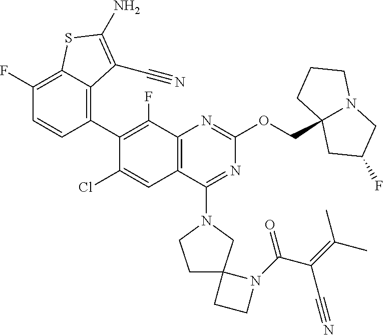 [SMILES: CC(C)=C(C#N)C(=O)N1CCC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-(1-(2-cyano-3- methylbut-2-enoyl)-1,6-diazaspiro[3.4]octan-6- yl)-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 747.3 |
| 298 | 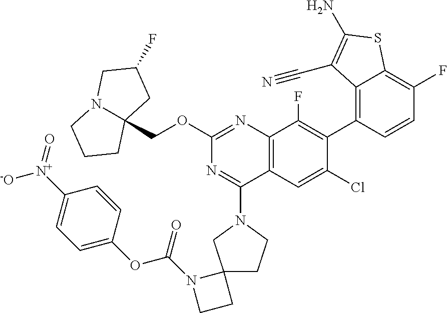 | 4-nitrophenyl 6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carboxylate | 805.2 |
| 299 ² | 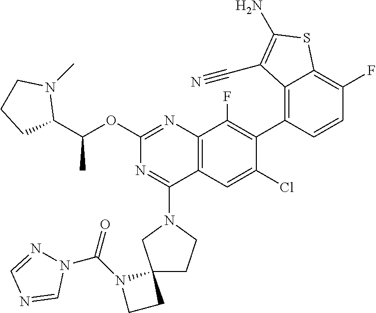 [SMILES: C[C@H](Oc1nc(N2CC[C@@]3(CCN3C(=O)n3cncn3)C2)c2cc(Cl)c(-c3ccc(F)c4sc(N)c(C#N)c34)c(F)c2n1)[C@@H]1CCCN1C] | 4-(4-((R)-1-(1H-1,2,4-triazole-1-carbonyl)-1,6- diazaspiro[3.4]octan-6-yl)-6-chloro-8-fluoro-2- ((S)-1-((S)-1-methylpyrrolidin-2- yl)ethoxy)quinazolin-7-yl)-2-amino-7- fluorobenzo[b]thiophene-3-carbonitrile | 705.2 |
| 300 ⁴ | 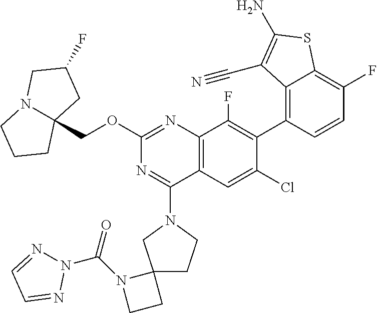 [SMILES: N#Cc1c(N)sc2c(F)ccc(-c3c(Cl)cc4c(N5CCC6(CCN6C(=O)n6nccn6)C5)nc(OC[C@@]56CCCN5C[C@H](F)C6)nc4c3F)c12] | 4-(4-(1-(2H-1,2,3-triazole-2-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.5 |
| 301 ³ | 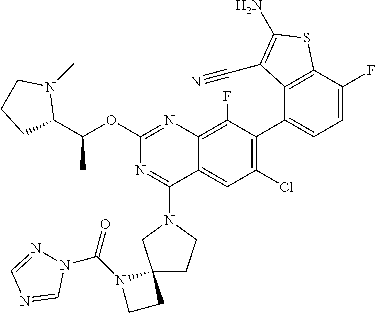 [SMILES: C[C@H](Oc1nc(N2CC[C@@]3(CCN3C(=O)n3cncn3)C2)c2cc(Cl)c(-c3ccc(F)c4sc(N)c(C#N)c34)c(F)c2n1)[C@@H]1CCCN1C] | 4-(4-((R)-1-(1H-1,2,4-triazole-1-carbonyl)-1,6- diazaspiro[3.4]octan-6-yl)-6-chloro-8-fluoro-2- ((S)-1-((S)-1-methylpyrrolidin-2- yl)ethoxy)quinazolin-7-yl)-2-amino-7- fluorobenzo[b]thiophene-3-carbonitrile | 705.2 |
| 302 | 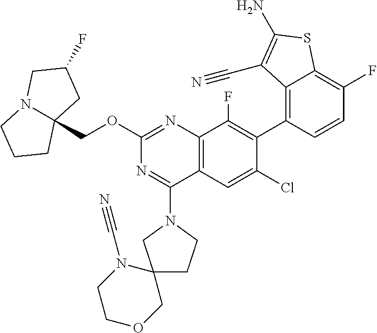 [SMILES: N#Cc1c(N)sc2c(F)ccc(-c3c(Cl)cc4c(N5CCC6(COCCN6C#N)C5)nc(OC[C@@]56CCCN5C[C@H](F)C6)nc4c3F)c12] | 2-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-9-oxa-2,6- diazaspiro[4.5]decane-6-carbonitrile | 695.4 |
| 303 | 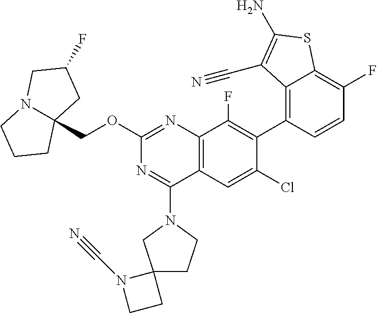 [SMILES: N#Cc1c(N)sc2c(F)ccc(-c3c(Cl)cc4c(N5CCC6(CCN6C#N)C5)nc(OC[C@@]56CCCN5C[C@H](F)C6)nc4c3F)c12] | 6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonitrile | 665.4 |
| 304 | 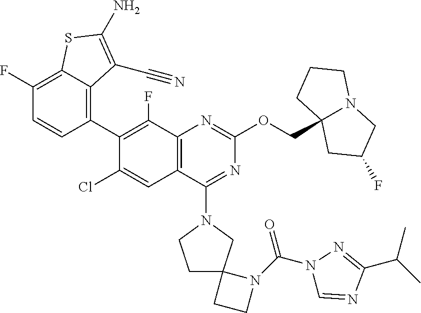 [SMILES: CC(C)c1ncn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-(1-(3-isopropyl-1H-1,2,4- triazole-1-carbonyl)-1,6-diazaspiro[3.4]octan-6- yl)quinazolin-7-yl)-7-fluorobenzo[b]thiophene- 3-carbonitrile | 777.4 |
| 305 ² | 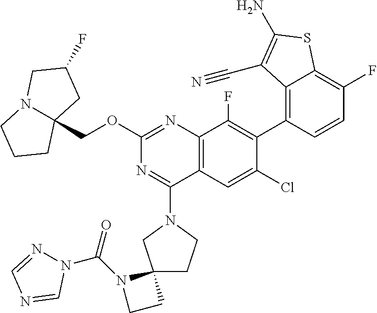 [SMILES: N#Cc1c(N)sc2c(F)ccc(-c3c(Cl)cc4c(N5CC[C@]6(CCN6C(=O)n6cncn6)C5)nc(OC[C@@]56CCCN5C[C@H](F)C6)nc4c3F)c12] | 4-(4-((S)-1-(1H-1,2,4-triazole-1-carbonyl)-1,6- diazaspiro[3.4]octan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 735.5 |
| 306 ² | 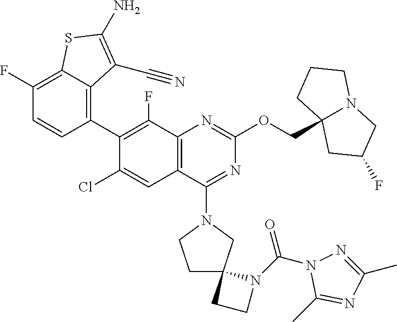 [SMILES: Cc1nc(C)n(C(=O)N2CC[C@]23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-4-((S)-1-(3,5-dimethyl- 1H-1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 763.3 |
| 307 | 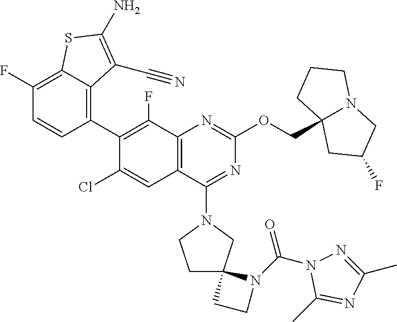 [SMILES: Cc1nc(C)n(C(=O)N2CC[C@@]23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-4-((R)-1-(3,5-dimethyl- 1H-1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 763.4 |
| 308 | 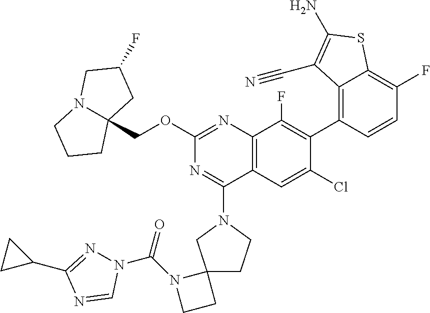 [SMILES: N#Cc1c(N)sc2c(F)ccc(-c3c(Cl)cc4c(N5CCC6(CCN6C(=O)n6cnc(C7CC7)n6)C5)nc(OC[C@@]56CCCN5C[C@H](F)C6)nc4c3F)c12] | 2-amino-4-(6-chloro-4-(1-(3-cyclopropyl-1H- 1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 775.3 |
| 309 | 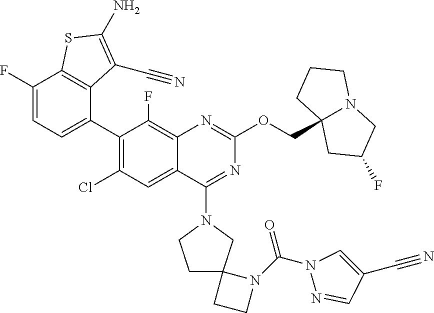 [SMILES: N#Cc1cnn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)c1] | 1-(6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonyl)-1H-pyrazole- 4-carbonitrile | 759.2 |
| 310 ⁵ | 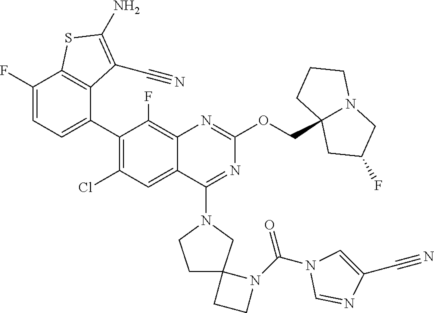 [SMILES: N#Cc1cn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)cn1] | 1-(6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonyl)-1H- imidazole-4-carbonitrile | 759.2 |
| 311 ⁵ | 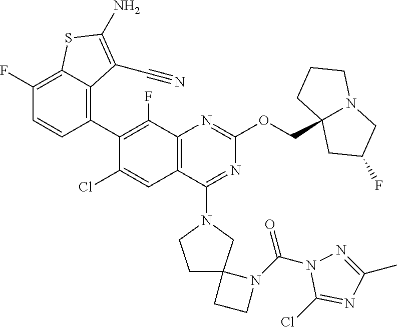 [SMILES: Cc1nc(Cl)n(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-4-(1-(5-chloro-3-methyl- 1H-1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 783.6 |
| 312 | 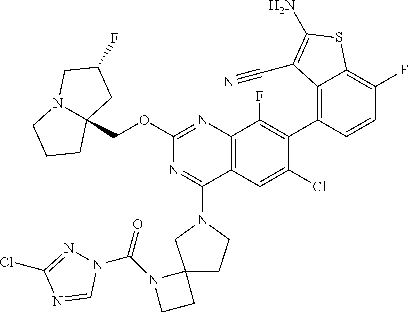 [SMILES: N#Cc1c(N)sc2c(F)ccc(-c3c(Cl)cc4c(N5CCC6(CCN6C(=O)n6cnc(Cl)n6)C5)nc(OC[C@@]56CCCN5C[C@H](F)C6)nc4c3F)c12] | 2-amino-4-(6-chloro-4-(1-(3-chloro-1H-1,2,4- triazole-1-carbonyl)-1,6-diazaspiro[3.4]octan-6- yl)-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 769.2 |
| 313 | 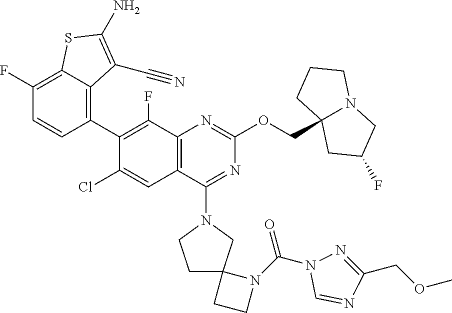 [SMILES: COCc1ncn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-(1-(3-(methoxymethyl)-1H- 1,2,4-triazole-1-carbonyl)-1,6- diazaspiro[3.4]octan-6-yl)quinazolin-7-yl)-7- fluorobenzo[b]thiophene-3-carbonitrile | 779.2 |
| 314 | 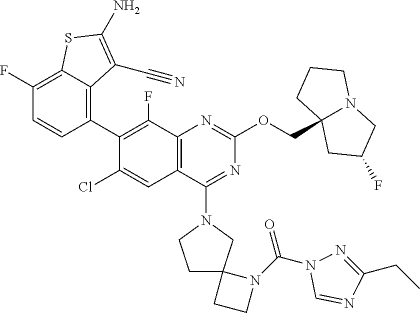 [SMILES: CCc1ncn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-4-(1-(3-ethyl-1H-1,2,4- triazole-1-carbonyl)-1,6-diazaspiro[3.4]octan-6- yl)-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 763.3 |
| 315 | 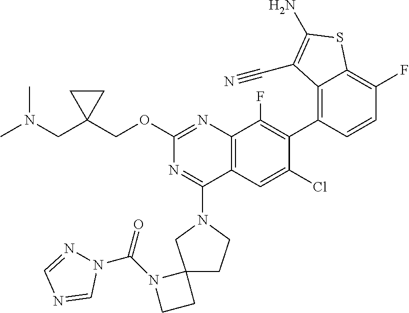 [SMILES: CN(C)CC1(COc2nc(N3CCC4(CCN4C(=O)n4cncn4)C3)c3cc(Cl)c(-c4ccc(F)c5sc(N)c(C#N)c45)c(F)c3n2)CC1] | 4-(4-(1-(1H-1,2,4-triazole-1-carbonyl)-1,6- diazaspiro[3.4]octan-6-yl)-6-chloro-2-((1- ((dimethylamino)methyl)cyclopropyl)methoxy)- 8-fluoroquinazolin-7-yl)-2-amino-7- fluorobenzo[b]thiophene-3-carbonitrile | 705.2 |
| 316 ² | 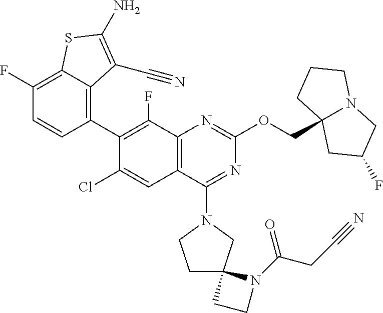 [SMILES: N#CCC(=O)N1CC[C@@]12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-4-((R)-1-(2-cyanoacetyl)- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 707.3 |
| 317 ⁶ | 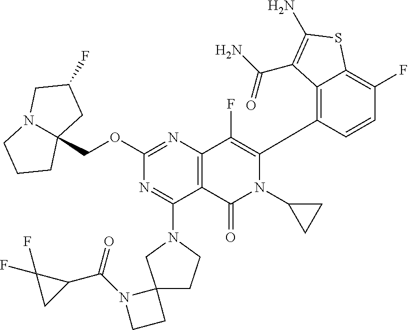 [SMILES: NC(=O)c1c(N)sc2c(F)ccc(-c3c(F)c4nc(OC[C@@]56CCCN5C[C@H](F)C6)nc(N5CCC6(CCN6C(=O)C6CC6(F)F)C5)c4c(=O)n3C3CC3)c12] | 2-amino-4-(6-cyclopropyl-4-(1-(2,2- difluorocyclopropane-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)-5-oxo-5,6- dihydropyrido[4,3-d]pyrimidin-7-yl)-7- fluorobenzo[b]thiophene-3-carboxamide | 784.4 |
| 318 ⁴ | 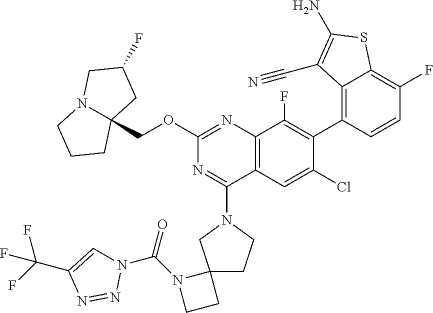 [SMILES: N#Cc1c(N)sc2c(F)ccc(-c3c(Cl)cc4c(N5CCC6(CCN6C(=O)n6cc(C(F)(F)F)nn6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-(1-(4-(trifluoromethyl)-1H-1,2,3- triazole-1-carbonyl)-1,6-diazaspiro[3.4]octan-6- yl)quinazolin-7-yl)-7-fluorobenzo[b]thiophene- 3-carbonitrile | 803.2 |
| 319 | 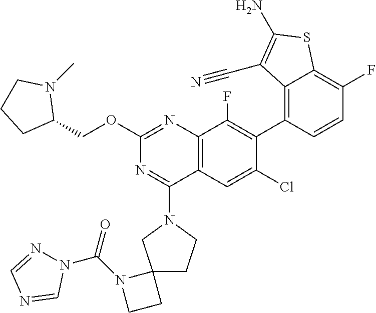 [SMILES: CN1CCC[C@H]1COc1nc(N2CCC3(CCN3C(=O)n3cncn3)C2)c2cc(Cl)c(-c3ccc(F)c4sc(N)c(C#N)c34)c(F)c2n1] | 4-(4-(1-(1H-1,2,4-triazole-1-carbonyl)-1,6- diazaspiro[3.4]octan-6-yl)-6-chloro-8-fluoro-2- (((S)-1-methylpyrrolidin-2- yl)methoxy)quinazolin-7-yl)-2-amino-7- fluorobenzo[b]thiophene-3-carbonitrile | 691.2 |
| 320 | 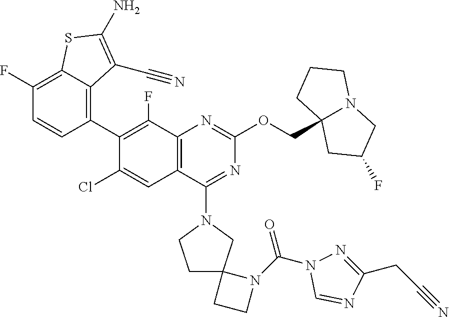 [SMILES: N#CCc1ncn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-4-(1-(3-(cyanomethyl)-1H- 1,2,4-triazole-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 774.3 |
| 321 ² | 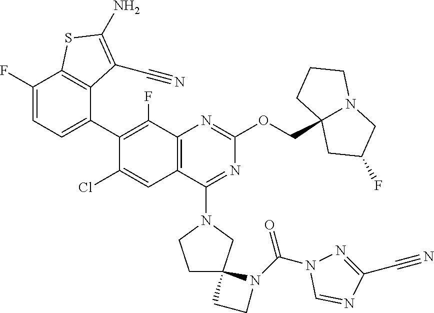 [SMILES: N#Cc1ncn(C(=O)N2CC[C@@]23CCN(c2nc(OC[C@@]45CCCN4C[C@H](F)C5)nc4c(F)c(-c5ccc(F)c6sc(N)c(C#N)c56)c(Cl)cc24)C3)n1] | 1-((4R)-6-(7-(2-amino-3-cyano-7- fluorobenzo[b]thiophen-4-yl)-6-chloro-8-fluoro- 2-(((2R,7aS)-2-fluorotetrahydro-1H-pyrrolizin- 7a(5H)-yl)methoxy)quinazolin-4-yl)-1,6- diazaspiro[3.4]octane-1-carbonyl)-1H-1,2,4- triazole-3-carbonitrile | 760.2 |
| 322 ² | 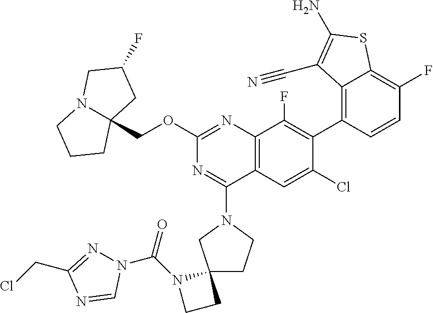 [SMILES: N#Cc1c(N)sc2c(F)ccc(-c3c(Cl)cc4c(N5CC[C@@]6(CCN6C(=O)n6cnc(CCl)n6)C5)nc(OC[C@@]56CCCN5C[C@H](F)C6)nc4c3F)c12] | 2-amino-4-(6-chloro-4-((R)-1-(3- (chloromethyl)-1H-1,2,4-triazole-1-carbonyl)- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 783.4 |
| 323 ^{2,6} | 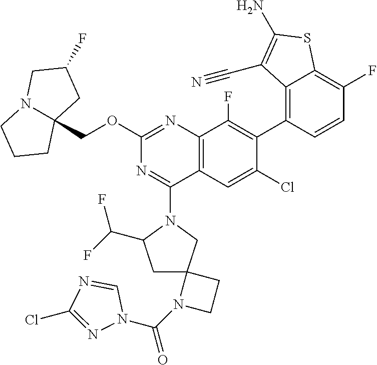 [SMILES: N#Cc1c(N)sc2c(F)ccc(-c3c(Cl)cc4c(N5CC6(CCN6C(=O)n6cnc(Cl)n6)CC5C(F)F)nc(OC[C@@]56CCCN5C[C@H](F)C6)nc4c3F)c12] | 2-amino-4-(6-chloro-4-(1-(3-chloro-1H-1,2,4- triazole-1-carbonyl)-7-(difluoromethyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 819.4 |
| 324 ² | 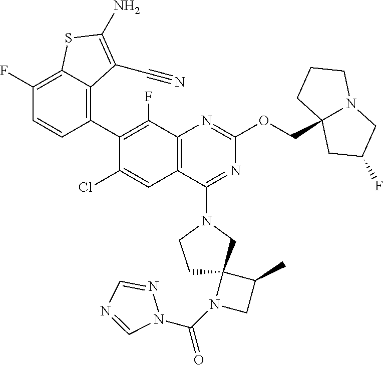 [SMILES: C[C@H]1CN(C(=O)n2cncn2)[C@]12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-((3S,4R)-3-methyl-1-(1H-1,2,4- triazole-1-carbonyl)-1,6-diazaspiro[3.4]octan-6- yl)quinazolin-7-yl)-7-fluorobenzo[b]thiophene- 3-carbonitrile | 749.4 |
| 325 ^{2,6} | 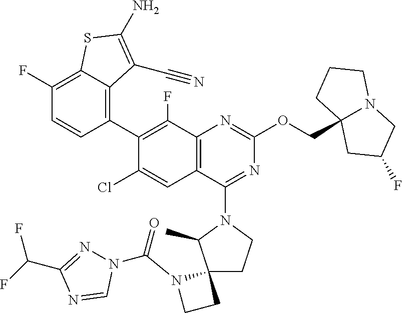 [SMILES: C[C@H]1N(c2nc(OC[C@@]34CCCN3C[C@H](F)C4)nc3c(F)c(-c4ccc(F)c5sc(N)c(C#N)c45)c(Cl)cc23)CC[C@@]12CCN2C(=O)n1cnc(C(F)F)n1] | 2-amino-4-(6-chloro-4-((4R,5R)-1-(3- (difluoromethyl)-1H-1,2,4-triazole-1-carbonyl)- 5-methyl-1,6-diazaspiro[3.4]octan-6-yl)-8- fluoro-2-(((2R,7aS)-2-fluorotetrahydro-1H- pyrrolizin-7a(5H)-yl)methoxy)quinazolin-7-yl)- 7-fluorobenzo[b]thiophene-3-carbonitrile | 799.1 |
| 326 ⁶ | 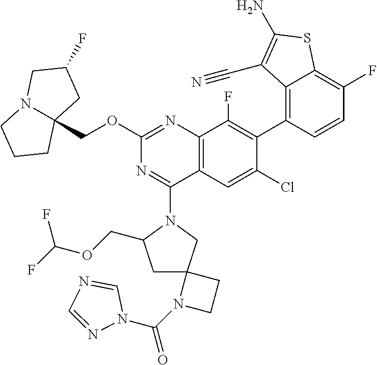 [SMILES: N#Cc1c(N)sc2c(F)ccc(-c3c(Cl)cc4c(N5CC6(CCN6C(=O)n6cncn6)CC5COC(F)F)nc(OC[C@@]56CCCN5C[C@H](F)C6)nc4c3F)c12] | 2-amino-4-(6-chloro-4-(7- ((difluoromethoxy)methyl)-1-(1H-1,2,4- triazole-1-carbonyl)-1,6-diazaspiro[3.4]octan-6- yl)-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 815.3 |
| 327 | 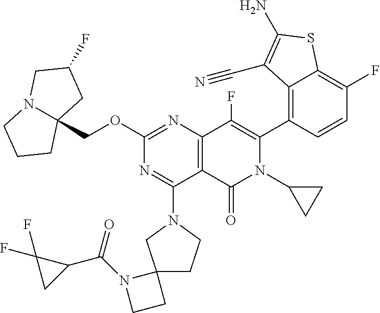 [SMILES: N#Cc1c(N)sc2c(F)ccc(-c3c(F)c4nc(OC[C@@]56CCCN5C[C@H](F)C6)nc(N5CCC6(CCN6C(=O)C6CC6(F)F)C5)c4c(=O)n3C3CC3)c12] | 2-amino-4-(6-cyclopropyl-4-(1-(2,2- difluorocyclopropane-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)-5-oxo-5,6- dihydropyrido[4,3-d]pyrimidin-7-yl)-7- fluorobenzo[b]thiophene-3-carbonitrile | 766.5 |
| 328 ² | 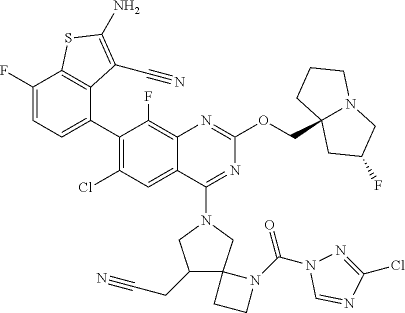 [SMILES: N#CCC1CN(c2nc(OC[C@@]34CCCN3C[C@H](F)C4)nc3c(F)c(-c4ccc(F)c5sc(N)c(C#N)c45)c(Cl)cc23)CC12CCN2C(=O)n1cnc(Cl)n1] | 2-amino-4-(6-chloro-4-(1-(3-chloro-1H-1,2,4- triazole-1-carbonyl)-8-(cyanomethyl)-1,6- diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 808.5 |
| 329 ^{2,6} | 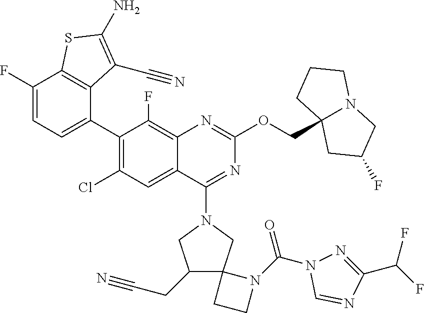 [SMILES: N#CCC1CN(c2nc(OC[C@@]34CCCN3C[C@H](F)C4)nc3c(F)c(-c4ccc(F)c5sc(N)c(C#N)c45)c(Cl)cc23)CC12CCN2C(=O)n1cnc(C(F)F)n1] | 2-amino-4-(6-chloro-4-(8-(cyanomethyl)-1-(3- (difluoromethyl)-1H-1,2,4-triazole-1-carbonyl)- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 824.1 |
| 330 ^{2,6} | 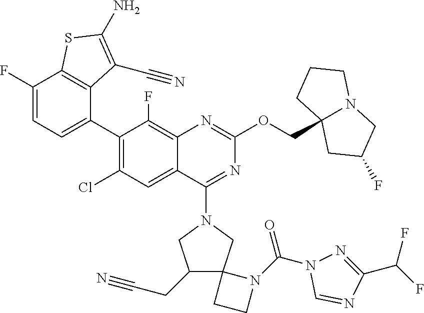 [SMILES: N#CCC1CN(c2nc(OC[C@@]34CCCN3C[C@H](F)C4)nc3c(F)c(-c4ccc(F)c5sc(N)c(C#N)c45)c(Cl)cc23)CC12CCN2C(=O)n1cnc(C(F)F)n1] | 2-amino-4-(6-chloro-4-(8-(cyanomethyl)-1-(3- (difluoromethyl)-1H-1,2,4-triazole-1-carbonyl)- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 824.2 |
| 331 ² | 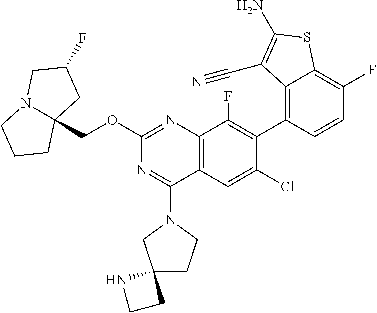 [SMILES: N#Cc1c(N)sc2c(F)ccc(-c3c(Cl)cc4c(N5CC[C@@]6(CCN6)C5)nc(OC[C@@]56CCCN5C[C@H](F)C6)nc4c3F)c12] | 2-amino-4-(6-chloro-8-fluoro-2-(((2R,7aS)-2- fluorotetrahydro-1H-pyrrolizin-7a(5H)- yl)methoxy)-4-((S)-1,6-diazaspiro[3.4]octan-6- yl)quinazolin-7-yl)-7-fluorobenzo[b]thiophene- 3-carbonitrile | 640.5 |
| 332 | 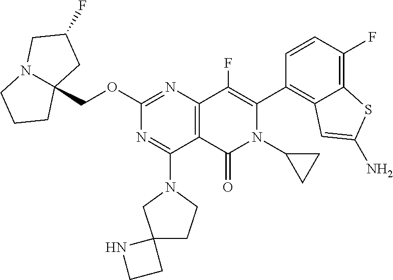 [SMILES: Nc1cc2c(-c3c(F)c4nc(OC[C@@]56CCCN5C[C@H](F)C6)nc(N5CCC6(CCN6)C5)c4c(=O)n3C3CC3)ccc(F)c2s1] | 7-(2-amino-7-fluorobenzo[d]thiazol-4-yl)-6- cyclopropyl-8-fluoro-2-(((2R,7aS)-2- fluorotetrahydro-1H-pyrrolizin-7a(5H)- yl)methoxy)-4-(1,6-diazaspiro[3.4]octan-6- yl)pyrido[4,3-d]pyrimidin-5(6H)-one | 638.4 |
| 333 | 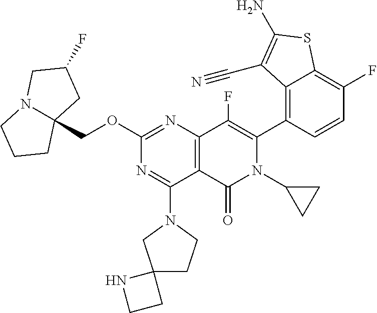 [SMILES: N#Cc1c(N)sc2c(F)ccc(-c3c(F)c4nc(OC[C@@]56CCCN5C[C@H](F)C6)nc(N5CCC6(CCN6)C5)c4c(=O)n3C3CC3)c12] | 2-amino-4-(6-cyclopropyl-8-fluoro-2- (((2R,7aS)-2-fluorotetrahydro-1H-pyrrolizin- 7a(5H)-yl)methoxy)-5-oxo-4-(1,6- diazaspiro[3.4]octan-6-yl)-5,6- dihydropyrido[4,3-d]pyrimidin-7-yl)-7- fluorobenzo[b]thiophene-3-carbonitrile | 662.4 |
| 334 ² | 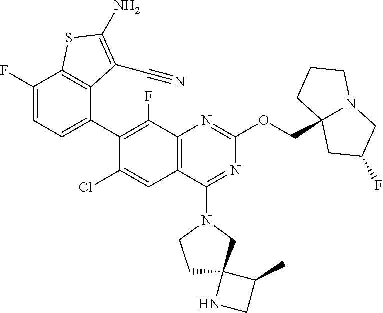 [SMILES: C[C@H]1CN[C@]12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-((3S,4R)-3-methyl-1,6- diazaspiro[3.4]octan-6-yl)quinazolin-7-yl)-7- fluorobenzo[b]thiophene-3-carbonitrile | 654.2 |
| 335 ² | 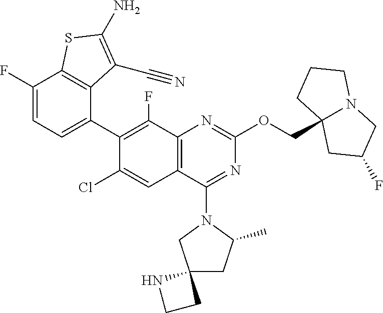 [SMILES: C[C@@H]1C[C@@]2(CCN2)CN1c1nc(OC[C@@]23CCCN2C[C@H](F)C3)nc2c(F)c(-c3ccc(F)c4sc(N)c(C#N)c34)c(Cl)cc12] | 2-amino-4-(6-chloro-8-fluoro-2-(((2R,7aS)-2- fluorotetrahydro-1H-pyrrolizin-7a(5H)- yl)methoxy)-4-((4S,7R)-7-methyl-1,6- diazaspiro[3.4]octan-6-yl)quinazolin-7-yl)-7- fluorobenzo[b]thiophene-3-carbonitrile | 654.2 |
| 336 ⁴ | 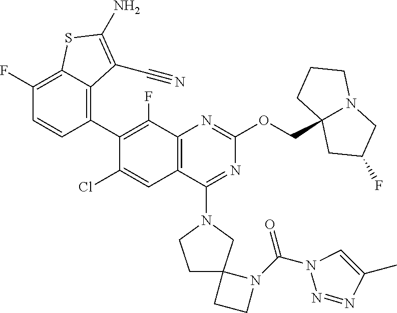 [SMILES: Cc1cn(C(=O)N2CCC23CCN(c2nc(OC[C@@]45CCCN4C[C@H](F)C5)nc4c(F)c(-c5ccc(F)c6sc(N)c(C#N)c56)c(Cl)cc24)C3)nn1] | 2-amino-4-(6-chloro-8-fluoro-2-(((2R,7aS)-2- fluorotetrahydro-1H-pyrrolizin-7a(5H)- yl)methoxy)-4-(1-(4-methyl-1H-1,2,3-triazole- 1-carbonyl)-1,6-diazaspiro[3.4]octan-6- yl)quinazolin-7-yl)-7-fluorobenzo[b]thiophene- 3-carbonitrile | 749.2 |
| 337 ⁵ | 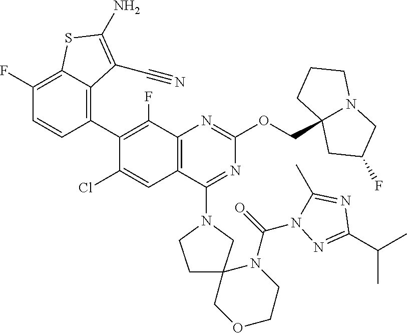 [SMILES: Cc1nc(C(C)C)nn1C(=O)N1CCOCC12CCN(c1nc(OC[C@@]34CCCN3C[C@H](F)C4)nc3c(F)c(-c4ccc(F)c5sc(N)c(C#N)c45)c(Cl)cc13)C2] | 2-amino-4-(6-chloro-8-fluoro-2-(((2R,7aS)-2- fluorotetrahydro-1H-pyrrolizin-7a(5H)- yl)methoxy)-4-(6-(3-isopropyl-5-methyl-1H- 1,2,4-triazole-1-carbonyl)-9-oxa-2,6- diazaspiro[4.5]decan-2-yl)quinazolin-7-yl)-7- fluorobenzo[b]thiophene-3-carbonitrile | 821.4 |
| 338 ^{2,4} | 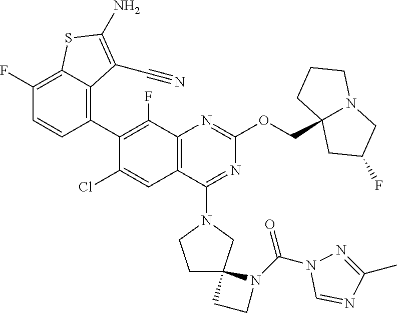 [SMILES: Cc1ncn(C(=O)N2CC[C@@]23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-((R)-1-(3-methyl-1H-1,2,4- triazole-1-carbonyl)-1,6-diazaspiro[3.4]octan-6- yl)quinazolin-7-yl)-7-fluorobenzo[b]thiophene- 3-carbonitrile | 749.4 |
| 339 ^{3,4} | 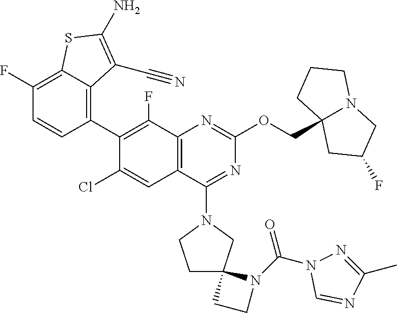 [SMILES: Cc1ncn(C(=O)N2CC[C@@]23CCN(c2nc(OC[C@@]45CCCN4C[C@H](F)C5)nc4c(F)c(-c5ccc(F)c6sc(N)c(C#N)c56)c(Cl)cc24)C3)n1] | 2-amino-4-(6-chloro-8-fluoro-2-(((2R,7aS)-2- fluorotetrahydro-1H-pyrrolizin-7a(5H)- yl)methoxy)-4-((R)-1-(3-methyl-1H-1,2,4- triazole-1-carbonyl)-1,6-diazaspiro[3.4]octan-6- yl)quinazolin-7-yl)-7-fluorobenzo[b]thiophene- 3-carbonitrile | 749.2 |
| 340 | 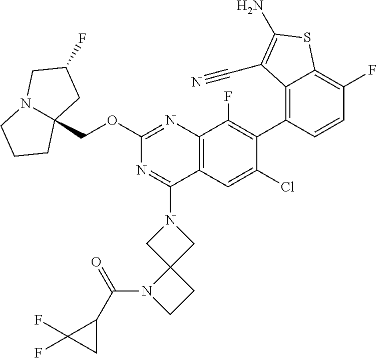 [SMILES: N#Cc1c(N)sc2c(F)ccc(-c3c(Cl)cc4c(N5CC6(CCN6C(=O)C6CC6(F)F)C5)nc(OC[C@@]56CCCN5C[C@H](F)C6)nc4c3F)c12] | 2-amino-4-(6-chloro-4-(1-(2,2- difluorocyclopropane-1-carbonyl)-1,6- diazaspiro[3.3]heptan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 730.3 |
| 341 | 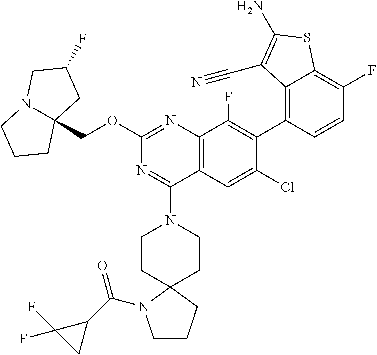 [SMILES: N#Cc1c(N)sc2c(F)ccc(-c3c(Cl)cc4c(N5CCC6(CCCN6C(=O)C6CC6(F)F)CC5)nc(OC[C@@]56CCCN5C[C@H](F)C6)nc4c3F)c12] | 2-amino-4-(6-chloro-4-(1-(2,2- difluorocyclopropane-1-carbonyl)-1,8- diazaspiro[4.5]decan-8-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 772.3 |
| 342 | 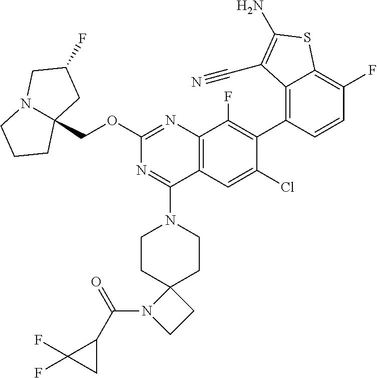 [SMILES: N#Cc1c(N)sc2c(F)ccc(-c3c(Cl)cc4c(N5CCC6(CC5)CCN6C(=O)C5CC5(F)F)nc(OC[C@@]56CCCN5C[C@H](F)C6)nc4c3F)c12] | 2-amino-4-(6-chloro-4-(1-(2,2- difluorocyclopropane-1-carbonyl)-1,7- diazaspiro[3.5]nonan-7-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 758.3 |
| 343 | 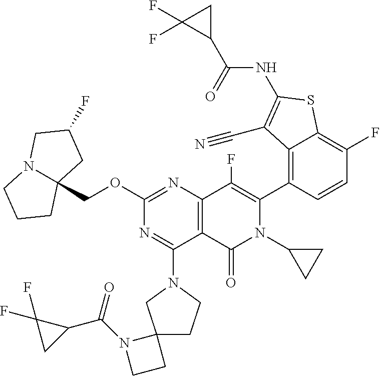 [SMILES: N#Cc1c(NC(=O)C2CC2(F)F)sc2c(F)ccc(-c3c(F)c4nc(OC[C@@]56CCCN5C[C@H](F)C6)nc(N5CCC6(CCN6C(=O)C6CC6(F)F)C5)c4c(=O)n3C3CC3)c12] | N-(3-cyano-4-(6-cyclopropyl-4-(1-(2,2- difluorocyclopropane-1-carbonyl)-1,6- diazaspiro[3.4]octan-6-yl)-8-fluoro-2- (((2R,7aS)-2-fluorotetrahydro-1H-pyrrolizin- 7a(5H)-yl)methoxy)-5-oxo-5,6- dihydropyrido[4,3-d]pyrimidin-7-yl)-7- fluorobenzo[b]thiophen-2-yl)-2,2- difluorocyclopropane-1-carboxamide | 870.5 |
| 344 | 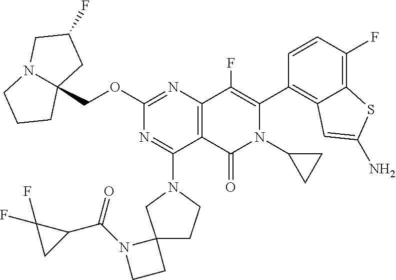 [SMILES: Nc1cc2c(-c3c(F)c4nc(OC[C@@]56CCCN5C[C@H](F)C6)nc(N5CCC6(CCN6C(=O)C6CC6(F)F)C5)c4c(=O)n3C3CC3)ccc(F)c2s1] | 7-(2-amino-7-fluorobenzo[d]thiazol-4-yl)-6- cyclopropyl-4-(1-(2,2-difluorocyclopropane-1- carbonyl)-1,6-diazaspiro[3.4]octan-6-yl)-8- fluoro-2-(((2R,7aS)-2-fluorotetrahydro-1H- pyrrolizin-7a(5H)-yl)methoxy)pyrido[4,3- d]pyrimidin-5(6H)-one | 742.4 |
| 345 | 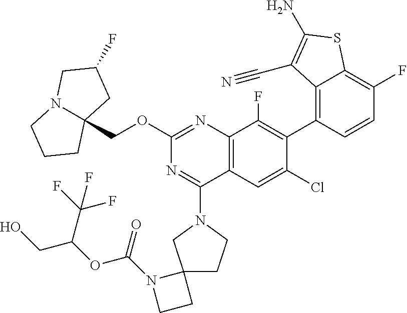 [SMILES: N#Cc1c(N)sc2c(F)ccc(-c3c(Cl)cc4c(N5CCC6(CCN6C(=O)OC(CO)C(F)(F)F)C5)nc(OC[C@@]56CCCN5C[C@H](F)C6)nc4c3F)c12] | 1,1,1-trifluoro-3-hydroxypropan-2-yl 6-(7-(2- amino-3-cyano-7-fluorobenzo[b]thiophen-4-yl)- 6-chloro-8-fluoro-2-(((2R,7aS)-2- fluorotetrahydro-1H-pyrrolizin-7a(5H)- yl)methoxy)quinazolin-4-yl)-1,6- diazaspiro[3.4]octane-1-carboxylate | 796.2 |
| 346 ³ | 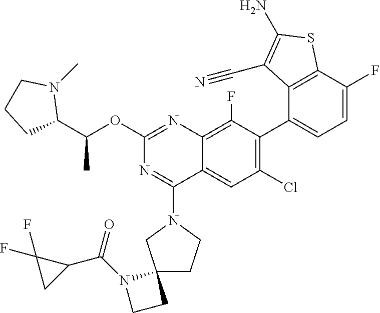 [SMILES: C[C@H](Oc1nc(N2CC[C@@]3(CCN3C(=O)C3CC3(F)F)C2)c2cc(Cl)c(-c3ccc(F)c4sc(N)c(C#N)c34)c(F)c2n1)[C@@H]1CCCN1C] | 2-amino-4-(6-chloro-4-((4R)-1-(2,2- difluorocyclopropane-1-carbonyl)-1,6- diazaspiro[3.4]octan-6-yl)-8-fluoro-2-((S)-1- ((S)-1-methylpyrrolidin-2-yl)ethoxy)quinazolin- 7-yl)-7-fluorobenzo[b]thiophene-3-carbonitrile | 714.3 |
| 347 | 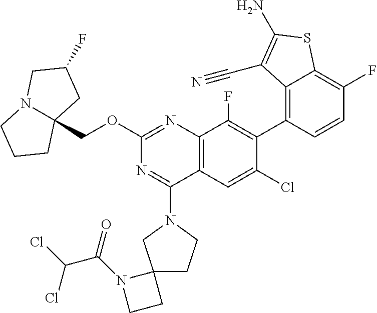 [SMILES: N#Cc1c(N)sc2c(F)ccc(-c3c(Cl)cc4c(N5CCC6(CCN6C(=O)C(Cl)Cl)C5)nc(OC[C@@]56CCCN5C[C@H](F)C6)nc4c3F)c12] | 2-amino-4-(6-chloro-4-(1-(2,2-dichloroacetyl)- 1,6-diazaspiro[3.4]octan-6-yl)-8-fluoro-2- (((2R,7aS)-2-fluorotetrahydro-1H-pyrrolizin- 7a(5H)-yl)methoxy)quinazolin-7-yl)-7- fluorobenzo[b]thiophene-3-carbonitrile | 751.1 |
| 348 ⁶ | 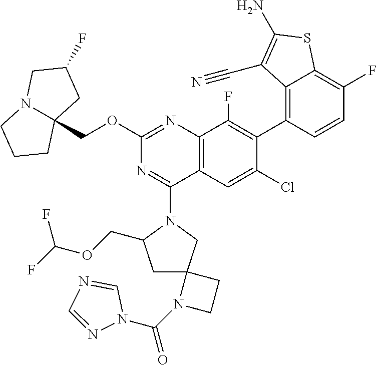 [SMILES: N#Cc1c(N)sc2c(F)ccc(-c3c(Cl)cc4c(N5CC6(CCN6C(=O)n6cncn6)CC5COC(F)F)nc(OC[C@@]56CCCN5C[C@H](F)C6)nc4c3F)c12] | 2-amino-4-(6-chloro-4-(7- ((difluoromethoxy)methyl)-1-(1H-1,2,4- triazole-1-carbonyl)-1,6-diazaspiro[3.4]octan-6- yl)-8-fluoro-2-(((2R,7aS)-2-fluorotetrahydro- 1H-pyrrolizin-7a(5H)-yl)methoxy)quinazolin-7- yl)-7-fluorobenzo[b]thiophene-3-carbonitrile | 815.2 |
| 349 ⁶ | 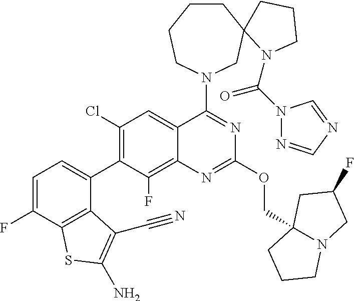 [SMILES: N#Cc1c(N)sc2c(F)ccc(-c3c(Cl)cc4c(N5CCCCC6(CCCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,7- diazaspiro[4.6]undecan-7-yl)-6-chloro-8-fluoro- 2-(((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 777.2 |
| 350 ² | 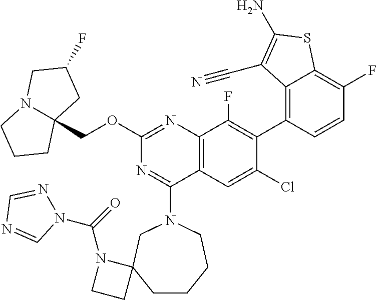 [SMILES: N#Cc1c(N)sc2c(F)ccc(-c3c(Cl)cc4c(N5CCCCC6(CCN6C(=O)n6cncn6)C5)nc(OC[C@@]56CCCN5C[C@H](F)C6)nc4c3F)c12] | 4-(4-(1-(1H-1,2,4-triazole-1-carbonyl)-1,6- diazaspiro[3.6]decan-6-yl)-6-chloro-8-fluoro-2- (((2R,7aS)-2-fluorotetrahydro-1H-pyrrolizin- 7a(5H)-yl)methoxy)quinazolin-7-yl)-2-amino-7- fluorobenzo[b]thiophene-3-carbonitrile | 763.1 |
| 351 ² | 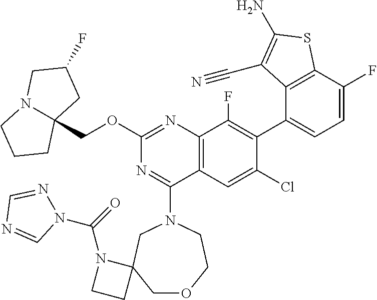 [SMILES: N#Cc1c(N)sc2c(F)ccc(-c3c(Cl)cc4c(N5CCOCC6(CCN6C(=O)n6cncn6)C5)nc(OC[C@@]56CCCN5C[C@H](F)C6)nc4c3F)c12] | 4-(4-(1-(1H-1,2,4-triazole-1-carbonyl)-6-oxa- 1,9-diazaspiro[3.6]decan-9-yl)-6-chloro-8- fluoro-2-(((2R,7aS)-2-fluorotetrahydro-1H- pyrrolizin-7a(5H)-yl)methoxy)quinazolin-7-yl)- 2-amino-7-fluorobenzo[b]thiophene-3- carbonitrile | 765.2 |

¹ compound provided as a single spirocyclic stereoisomer and mixture of atropisomers
² compound provided as a single atropisomer (R)
³ compound provided as a single atropisomer (S)
⁴ Compound provided as a mixture of regioisomers (e.g., of optionally substituted imidazole, pyrazole, triazole, or tetrazole)
⁵ Compound provided as a substantially pure single regioisomer (e.g., of optionally substituted imidazole, pyrazole, triazole, or tetrazole), though the structure provided has been tentatively assigned
⁶ Compound provided as partially purified mixture of diastereomers and atropisomers
⁷ Compound provided as tentatively assigned single diastereomer and single atropisomer
It will be understood that when referring to one or more atropisomers of the compounds in the table above, the atropisomer is in reference to the orientation of the bonds attached to the atom of the substituent corresponding to the R¹⁷/R¹⁹ group of Formula (I) that is bonded to the V atom of Formula (I).
It will be understood that when two superscripted numbers above (e.g, 2 and 6) apply to a single compound and both numbers limit the same characteristic (e.g., atropisomer), the number with greater specificity will apply. For example, when a compound is classified as 2 and 6, the compound is a partially purified mixture of diastereomers and a single R atropisomer.

Unless indicated otherwise, when a compound of Table 1 may exist as atropisomers or diastereomers and a single isomer or limited mixture of isomers is not described in the chemical structure, name, or superscripted notes of Table 1, it will be understood that the compound may be provided as a mixture of two or more isomers. When two compounds are provided with identical chemical structures and/or names in Table 1 and the superscripted note for each compound indicates a single atropisomer or partially purified mixture of isomers (e.g., diastereomers) without identifying the exact atropisomer or isomer (e.g., diastereomer), it will be understood that the two compounds are provided as different atropisomers, isomers (e.g., diastereomers), or partially purified mixtures of isomers.

In some instances, particularly for compounds denoted with 4, two or more regioisomers may exist and the compound structures and corresponding chemical names provided have been tentatively assigned. In the preparation of certain compounds herein, such as compounds comprising a pyrazolyl, imidazolyl, triazolyl, or tetrazolyl, the starting material may exist in two or more tautomeric forms, thus the products may be isolated from the respective reaction mixtures as a single regioisomer or as a mixture of regioisomers that result from reaction with the two or more tautomeric forms of the starting material.

In some instances, particularly for compounds denoted with 5, one substantially pure regioisomer is provided and the compound structures and corresponding chemical names provided are tentatively assigned.

Unless indicated otherwise, where atropisomers of a compound are possible, the compound may be provided as a mixture of atropisomers. Unless indicated otherwise, where a compound includes a possible stereocenter (e.g., spirocyclic stereocenter) and no specific stereoisomer(s) is depicted, the compound may be provided as a mixture of stereoisomers.

For example, Compound 214 was isolated and screened as a mixture of the two regioisomers depicted below, though for convenience only one regioisomer is depicted in Table 1:

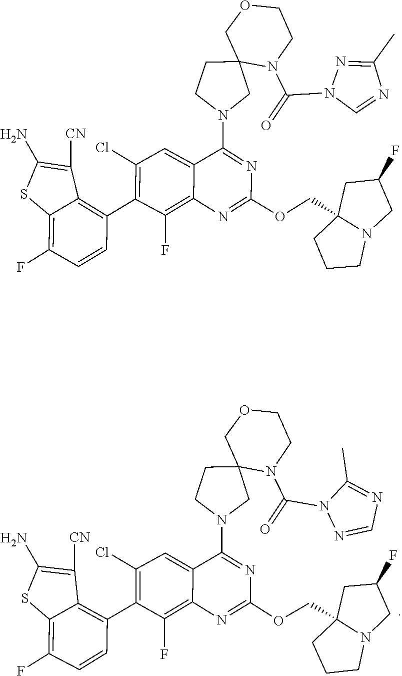 [SMILES: Cc1ncn(C(=O)N2CCOCC23CCN(c2nc(OC[C@@]45CCCN4C[C@H](F)C5)nc4c(F)c(-c5ccc(F)c6sc(N)c(C#N)c56)c(Cl)cc24)C3)n1 ; Cc1ncnn1C(=O)N1CCOCC12CCN(c1nc(OC[C@@]34CCCN3C[C@H](F)C4)nc3c(F)c(-c4ccc(F)c5sc(N)c(C#N)c45)c(Cl)cc13)C2]

### Example 2: Ras Sequences

| Human K-Ras G12S (SEQ ID NO. 1): | |
|---|---|
| 1 | MTEYKLVVVG ASGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human K-Ras Wildtype sequence | |
| (SEQ ID NO. 2) | |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| H-Ras G12S | |
| (SEQ ID NO. 3) | |
| 1 | MTEYKLVVVG ASGVGKSALT IQLIQNHFVD EYDPTIEDSY RKQVVIDGET |
| 51 | CLLDILDTAG QEEYSAMRDQ YMRTGEGFLC VFAINNTKSF EDIHQYREQI |
| 101 | KRVKDSDDVP MVLVGNKCDL AARTVESRQA QDLARSYGIP YIETSAKTRQ |
| 151 | GVEDAFYTLV REIRQHKLRK LNPPDESGPG CMSCKCVLS |
| H-Ras wildtype | |
| (SEQ ID NO. 4) | |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHQYREQI |
| 101 | KRVKDSDDVP MVLVGNKCDL AARTVESRQA QDLARSYGIP YIETSAKTRQ |
| 151 | GVEDAFYTLV REIRQHKLRK LNPPDESGPG CMSCKCVLS |
| Human N-Ras G12S | |
| (SEQ ID NO. 5) | |
| 1 | MTEYKLVVVG ASGVGKSALT IQLIQNHFVD EYDPTIEDSY RKQVVIDGET |
| 51 | CLLDILDTAG QEEYSAMRDQ YMRTGEGFLC VFAINNSKSF ADINLYREQI |
| 101 | KRVKDSDDVP MVLVGNKCDL PTRTVDTKQA HELAKSYGIP FIETSAKTRQ |
| 151 | GVEDAFYTLV REIRQYRMKK LNSSDDGTQG CMGLPCVVM |
| Human N-Ras wildtype | |
| (SEQ ID NO. 6) | |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNSKSF ADINLYREQI |
| 101 | KRVKDSDDVP MVLVGNKCDL PTRTVDTKQA HELAKSYGIP FIETSAKTRQ |
| 151 | GVEDAFYTLV REIRQYRMKK LNSSDDGTQG CMGLPCVVM |
| Human K-Ras G12D | |
| (SEQ ID NO. 7) | |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human K-Ras G12V | |
| (SEQ ID NO. 8) | |
| 1 | MTEYKLVVVG AVGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |

### Example 3: Protein Expression

DNA expression constructs encoding one or more protein sequences of interest (e.g., Kras fragments thereof, mutant variants thereof, etc.) and its corresponding DNA sequences are optimized for expression in E. coli and synthesized by, for example, the GeneArt Technology at Life Technologies. In some cases, the protein sequences of interest are fused with a tag (e.g., glutathione S-transferase (GST), histidine (His), or any other affinity tags) to facilitate recombinant expression and purification of the protein of interest. Such tag can be cleaved subsequent to purification. Alternatively, such tag may remain intact to the protein of interest and may not interfere with activities (e.g., target binding and/or phosphorylation) of the protein of interest

A resulting expression construct is additionally encoded with (i) att-site sequences at the 5′ and 3′ ends for subcloning into various destination vectors using, for example, the Gateway Technology, as well as (ii) a Tobacco Etch Virus (TEV) protease site for proteolytic cleavage of one or more tag sequences. The applied destination vectors can be a pET vector series from Novagen (e.g., with ampicillin resistance gene), which provides an N-terminal fusion of a GST-tag to the integrated gene of interest and/or a pET vector series (e.g., with ampicillin resistance gene), which provides a N-terminal fusion of a HIS-tag to the integrated gene. To generate the final expression vectors, the expression construct of the protein of interest is cloned into any of the applied destination ventors. The expression vectors are transformed into E. coli strain, e.g., BL21 (DE3). Cultivation of the transformed strains for expression is performed in 10 L and 1 L fermenter. The cultures are grown, for example, in Terrific Broth media (MP Biomedicals, Kat. #1 13045032) with 200 ug/mL ampicillin at a temperature of 37° C. to a density of 0.6 (OD600), shifted to a temperature of ˜27° C. (for K-Ras expression vectors) induced for expression with 100 mM IPTG, and further cultivated for 24 hours. After cultivation, the transformed E. coli cells are harvested by centrifugation and the resulting pellet is suspended in a lysis buffer, as provided below, and lysed by passing three-times through a high pressure device. The lysate is centrifuged (49000 g, 45 min, 4° C.) and the supernatant is used for further purification.

### Example 4: Ras protein purification

A Ras (e.g., K-Ras wildtype or a mutant such as K-Ras G12S, K-Ras G12D, K-Ras G12V, K-Ras G12C, K-Ras G13D, K-Ras G13C, or K-Ras G13V construct or a variant thereof is tagged with GST. E. coli culture from a 10 L fermenter is lysed in lysis buffer (50 mM Tris HCl 7.5, 500 mM NaCl, 1 mM DTT, 0.5% CHAPS, Complete Protease Inhibitor Cocktail-(Roche)). As a first chromatography step, the centrifuged lysate is incubated with 50 mL Glutathione Agarose 4B (Macherey-Nagel; 745500.100) in a spinner flask (16 h, 10O). The Glutathione Agarose 4B loaded with protein is transferred to a chromatography column connected to a chromatography system, e.g., an Akta chromatography system. The column is washed with wash buffer (50 mM Tris HCl 7.5, 500 mM NaCl, 1 mM DTT) and the bound protein is eluted with elution buffer (50 mM Tris HCl 7.5, 500 mM NaCl, 1 mM DTT, 15 mM Glutathione). The main fractions of the elution peak (monitored by OD280) is pooled. For further purification by size-exclusion chromatography, the above eluate volume is applied to a column Superdex 200 HR prep grade (GE Healthcare) and the resulting peak fractions of the eluted fusion protein is collected. Native mass spectrometry analyses of the final purified protein construct can be performed to assess its homogeneous load with GDP.

### Example 5: HTRF (Homogenous Time-Resolved Fluorescence Resonance Energy Transfer Assay

The ability of a compound of the present disclosure to reduce a Ras signaling output can be demonstrated by an HTRF assay. This assay can be also used to assess a selective inhibition or reduction of signaling output of a mutant Ras protein relative to a wildtype, or relative to a different mutant Ras protein. For example, the equilibrium interaction of wildtype Kras or K-Ras mutant (e.g., wildtype or a mutant thereof including those mentioned in Example 4) with SOS1 (e.g., hSOS1) can be assessed as a proxy or an indication for a subject compound's ability to bind and inhibit Ras protein. HTRF assay detects from (i) a fluorescence resonance energy transfer (FRET) donor (e.g., antiGST-Europium) that is bound to GST-tagged K-Ras mutant to (ii) a FRET acceptor (e.g., anti-6His-XL665) bound to a His-tagged hSOS1.

The assay buffer can contain ˜5 mM HEPES pH 7.4, ˜150 mM NaCl, ˜1 mM DTT, 0.05% BSA and 0.0025% (v/v) Igepal. A Ras working solution is prepared in an assay buffer containing typically a suitable amount of the protein construct (e.g., GST-tagged K-Ras mutant) and the FRET donor (e.g., antiGST-Eu(K) from Cisbio, France). A SOS1 working solution is prepared in an assay buffer containing suitable amount of the protein construct (e.g., His-hSOS1) and the FRET acceptor (e.g., anti-6His-XL665 from Cisbio, France). A suitable amount of the protein construct will depend on the range of activity or range of IC50 values being detected or under investigation. For detecting IC50 within a range of 500 nM, the protein constructs of the same range of molarity can be utilized. An inhibitor control solution is prepared in an assay buffer containing comparable amount of the FRET acceptor without the SOS1 protein. A fixed volume of DMSO with or without test compound is transferred into a 384-well plate. Ras working solution is added to all wells of the test plate. SOS1 working solution is added to all wells except for those that are subsequently filled the inhibitor control solution. Upon incubation for about 10 minutes or longer, the fluorescence is measured with a M1000Pro plate reader (Tecan) using HTRF detection (excitation 337 nm, emission 1: 620 nm, emission 2: 665 nm). Compounds are tested in duplicates at different concentrations (for example, 10 μM, 2.5 μM, 0.63 μM, 0.16 μM, 0.04 μM, 0.01 μM test compound). The ratiometric data (i.e., emission 2 divided by emission 1) is used to calculate IC50 values against Ras using GraphPad Prism (GraphPad software). Following this general procedure, samples were tested with or without a subject compound disclosed herein including compounds exemplified in Table 1 to assess their abilities to inhibit a mutant K-Ras relative to another mutant K-Ras or wildtype K-Ras. Signaling output measured in terms of IC50 values can be obtained, a ratio of IC50 against one mutant relative to another mutant can be calculated. For instance, a selective reduction of K-Ras G12D signaling output can be evidenced by a ratio greater than one. In particular, a selective reduction of K-Ras G12D signaling relative to K-Ras WT signaling is evidenced as the ratio of IC50 (against K-Ras WT) to IC50 (against K-Ras G12D) is greater than 1. In some examples, one or more subject compounds (including without limitation compounds 206, 249, 254, 256, 264, 271, and 276) exhibited selective inhibition of K-Ras G12S relative to wildtype or a different mutant (e.g., K-Ras G12D) as evidenced by a ratio of IC50 against K-Ras G12D to that of K-Ras G12S being greater than 1. In other examples, one or more subject compounds (including without limitation compounds 204, 212, 218, 219, 230, and 265) exhibited selective inhibition of K-Ras G12D relative to wildtype or a different mutant (e.g., K-Ras G12S) as evidenced by a ratio of IC50 against K-Ras G12S to that of K-Ras G12D being greater than 1.

TABLE 2
HTRF assay results using an assay
as generally described above.

| | PPI- | PPI- | PPI- |
|---|---|---|---|
| | HTRF- | HTRF- | HTRF- |
| | 5 nM | 5 nM | 5 nM |
| Compound | KRAS | KRAS | KRAS |
| No. | G12S | G12D | WT |
| 201 | +++ | +++ | +++ |
| 202 | +++ | +++ | +++ |
| 203 | +++ | ++ | +++ |
| 204 | +++ | +++ | +++ |
| 205 | +++ | +++ | +++ |
| 206 | +++ | +++ | +++ |
| 207 | +++ | +++ | +++ |
| 208 | +++ | +++ | +++ |
| 209 | +++ | +++ | +++ |
| 210 | +++ | +++ | +++ |
| 211 | +++ | +++ | +++ |
| 212 | +++ | +++ | +++ |
| 213 | +++ | +++ | +++ |
| 214 | +++ | +++ | +++ |
| 215 | +++ | +++ | +++ |
| 216 | +++ | +++ | +++ |
| 217 | +++ | +++ | +++ |
| 218 | +++ | +++ | +++ |
| 219 | +++ | +++ | +++ |
| 220 | ND | +++ | +++ |
| 221 | +++ | +++ | +++ |
| 222 | +++ | +++ | +++ |
| 223 | +++ | +++ | +++ |
| 224 | +++ | +++ | +++ |
| 225 | +++ | +++ | +++ |
| 226 | +++ | +++ | +++ |
| 227 | +++ | +++ | +++ |
| 228 | +++ | +++ | +++ |
| 229 | +++ | +++ | +++ |
| 230 | +++ | +++ | +++ |
| 231 | +++ | +++ | +++ |
| 232 | +++ | +++ | +++ |
| 233 | +++ | +++ | +++ |
| 234 | +++ | +++ | +++ |
| 235 | +++ | +++ | +++ |
| 236 | +++ | +++ | +++ |
| 237 | +++ | ++ | +++ |
| 238 | +++ | +++ | +++ |
| 239 | +++ | ++ | +++ |
| 240 | +++ | +++ | +++ |
| 241 | +++ | +++ | +++ |
| 242 | +++ | +++ | +++ |
| 243 | +++ | +++ | +++ |
| 244 | +++ | +++ | +++ |
| 245 | ++ | ++ | ++ |
| 246 | +++ | +++ | +++ |
| 247 | +++ | +++ | +++ |
| 248 | +++ | +++ | +++ |
| 249 | +++ | +++ | +++ |
| 250 | +++ | +++ | +++ |
| 251 | ++ | +++ | +++ |
| 252 | +++ | +++ | +++ |
| 253 | ++ | ++ | +++ |
| 254 | +++ | +++ | +++ |
| 255 | +++ | +++ | +++ |
| 256 | +++ | ++ | ++ |
| 257 | +++ | +++ | +++ |
| 258 | +++ | +++ | +++ |
| 259 | +++ | +++ | +++ |
| 260 | +++ | +++ | +++ |
| 261 | +++ | +++ | +++ |
| 262 | +++ | +++ | +++ |
| 263 | +++ | +++ | +++ |
| 264 | +++ | +++ | +++ |
| 265 | ++ | +++ | +++ |
| 266 | +++ | +++ | +++ |
| 267 | +++ | +++ | +++ |
| 268 | +++ | +++ | +++ |
| 269 | +++ | +++ | +++ |
| 270 | +++ | +++ | +++ |
| 271 | +++ | +++ | +++ |
| 272 | +++ | +++ | +++ |
| 273 | +++ | +++ | +++ |
| 274 | +++ | +++ | +++ |
| 275 | +++ | +++ | +++ |
| 276 | +++ | +++ | +++ |
| 277 | +++ | +++ | +++ |
| 278 | +++ | +++ | +++ |
| 279 | ++ | ++ | ++ |
| 280 | +++ | +++ | +++ |
| 281 | +++ | +++ | +++ |
| 282 | +++ | +++ | +++ |
| 283 | +++ | +++ | +++ |
| 284 | +++ | +++ | +++ |
| 285 | +++ | +++ | +++ |
| 286 | +++ | +++ | +++ |
| 287 | +++ | +++ | +++ |
| 288 | +++ | +++ | +++ |
| 289 | +++ | +++ | +++ |
| 290 | +++ | +++ | +++ |
| 291 | +++ | +++ | +++ |
| 292 | +++ | +++ | +++ |
| 293 | +++ | +++ | +++ |
| 294 | +++ | +++ | +++ |
| 295 | +++ | +++ | +++ |
| 296 | +++ | +++ | +++ |
| 297 | +++ | +++ | +++ |
| 298 | +++ | +++ | +++ |
| 299 | +++ | +++ | +++ |
| 300 | +++ | +++ | +++ |
| 301 | +++ | +++ | +++ |
| 302 | +++ | +++ | +++ |
| 303 | +++ | +++ | +++ |
| 304 | +++ | +++ | +++ |
| 305 | +++ | +++ | +++ |
| 306 | +++ | +++ | +++ |
| 307 | +++ | +++ | +++ |
| 308 | +++ | +++ | +++ |
| 309 | +++ | +++ | +++ |
| 310 | +++ | +++ | +++ |
| 311 | +++ | +++ | +++ |
| 312 | +++ | +++ | +++ |
| 313 | +++ | +++ | +++ |
| 314 | +++ | +++ | +++ |
| 315 | +++ | +++ | +++ |
| 316 | +++ | +++ | +++ |
| 317 | +++ | +++ | +++ |
| 318 | +++ | +++ | +++ |
| 319 | +++ | +++ | +++ |
| 320 | +++ | +++ | +++ |
| 321 | +++ | +++ | +++ |
| 322 | +++ | +++ | +++ |
| 323 | +++ | ++ | ++ |
| 324 | +++ | +++ | +++ |
| 325 | +++ | +++ | +++ |
| 326 | +++ | +++ | +++ |
| 327 | +++ | +++ | +++ |
| 328 | +++ | +++ | +++ |
| 329 | +++ | +++ | +++ |
| 330 | +++ | +++ | +++ |
| 331 | +++ | +++ | +++ |
| 332 | +++ | +++ | +++ |
| 333 | +++ | +++ | +++ |
| 334 | +++ | +++ | +++ |
| 335 | +++ | +++ | +++ |
| 336 | +++ | +++ | +++ |
| 337 | +++ | +++ | +++ |
| 338 | +++ | +++ | +++ |
| 339 | +++ | +++ | +++ |
| 340 | +++ | +++ | +++ |
| 341 | +++ | +++ | +++ |
| 342 | +++ | +++ | +++ |
| 343 | +++ | +++ | +++ |
| 344 | +++ | +++ | +++ |
| 345 | +++ | +++ | +++ |
| 346 | +++ | +++ | +++ |
| 347 | +++ | +++ | +++ |
| 348 | +++ | +++ | +++ |
| 349 | +++ | +++ | +++ |
| 350 | +++ | +++ | +++ |
| 351 | +++ | +++ | +++ |

++ IC50 greater than 5 μM;
+++ IC50 less than or equal to 5 μM

### Example 6: GTPase Activity Assay

The ability of any compound of the present disclosure to inhibit a Ras protein signaling can be demonstrated by a reduced GTPase activity. This assay can be also used to assess a selective inhibition of a mutant Ras protein relative to a wildtype, or relative to a different mutant Ras protein. For instance, the assay can be used to establish a subject compound's ability to selectively inhibit Kras G12D relative to wildtype, G12S relative to wildtype, Kras G12V relative to wildtype, KrasG12S relative KrasG12V, KrasG12S relative KrasG12D, KrasG12D relative to KrasG12S, or KrasG12D relative KrasG12V, or vice versa. In particular, intrinsic and GTPase-activating protein (GAP)-stimulated GTPase activity for K-Ras construct or a mutant thereof can be measured using EnzCheck phosphate assay system (Life Technologies). For example K-Ras WT, K-Ras D154Q mutant, K-Ras G12D mutant, K-Ras G12S mutant, and K-Ras G12D/D154Q mutant proteins (2.5 mg/ml) in buffer (20 mmol/L Tris, pH 8.0, 50 mM NaCl) is loaded with GTP at room temperature for 2 hours by exposing to exchange buffer containing EDTA. Proteins are buffer exchanged to assay buffer (30 mM Tris, pH 7.5, 1 mM DTT) and the concentration is adjusted to 2 mg/ml. GTP loading is verified by back extraction of nucleotide using 6M urea and evaluation of nucleotide peaks by HPLC using an ion-exchange column. The assay is performed in a clear 384-well plate (Costar) by combining GTP-loaded K-Ras proteins (50 mM final) with 2-amino-6-mercapto-7-methylpurine ribonucleoside (MESG) (200 mM final), and purine nucleotide phosphorylase (5 U/ml final). GTP hydrolysis is initiated by the addition of MgCl2 at a working concentration of 40 mM. For GAP stimulation, Ras p21 protein activator 1 (P120GAP) can be included at 50 mM. Absorbance at 360 nm can be measured every 8 to 15 s for 1,000 s at 20° C. Samples are tested with or without a subject compound disclosed herein including compounds exemplified in Table 1 to assess each compound's ability to inhibit signaling of a given Ras protein (e.g., a given mutant Kras) of interest.

### Example 7: Nucleotide Exchange Assay

The ability of a compound of the present disclosure to inhibit a Ras protein signaling can be demonstrated by a reduced nucleotide exchange activity. This assay can be also used to assess a selective inhibition of a mutant Ras protein relative to a wildtype, or relative to a different mutant Ras protein. For example, 250 nM or 500 nM GDP-loaded K-Ras proteins (e.g., wildtype or a mutant thereof including those mentioned in Example 6), each is incubated with different concentrations of compounds (for example ˜60 μM, ˜20 μM, ˜6.7 μM, ˜2.2 μM, ˜0.7 μM, ˜0.2 μM subject compound). A control reaction without subject compound is also included. SOS1 (catalytic domain) protein is added to the K-Ras protein solution. The nucleotide exchange reaction is initiated by adding fluorescent labelled GDP (Guanosine 5′-Diphosphate, BODIPY™ FL 2′-(or-3′)—O—(N-(2-Aminoethyl) Urethane) to a final concentration of 0.36 μM. Fluorescence is measured every 30 s for 70 minutes at 490 nm/515 nm (excitation/emission) in a M1000Pro plate reader (Tecan). Data is exported and analyzed to calculate an IC50 using GraphPad Prism (GraphPad Software). Sample(s) can be tested with or without a subject compound disclosed herein including compound(s) exemplified in Table 1 to assess compound's ability to inhibit K-Ras signaling or its IC50 against a given Ras protein (e.g., a given mutant K-Ras) of interest.

### Example 8: Testing for Modification of Ras Protein

Test compounds are prepared as 10 mM stock solutions in DMSO (Fisher cat #BP231-100). KRAS protein (e.g., His-tagged GDP-loaded wildtype 1-169, His-tagged GDP-loaded G12S1-169, His-tagged GDP-loaded G12D 1-169, or His-tagged GDP-loaded G12C 1-169) is diluted to ˜2 μM in appropriate buffer (e.g., a Hepes buffer at physiological conditions). For testing KRAS modification, compounds are diluted to 50× final test concentration in DMSO in 96-well storage plates. 2 μl of the diluted 50× compounds are added to appropriate wells in the PCR plate (Fisher cat #AB-0800). ˜49 μl of the stock protein solution is added to each well of the 96-well PCR plate. Reactions are mixed carefully. The plate is sealed well with aluminum plate seal, and stored in drawer at room temperature for 24 hrs. 5 μl of 2% formic acid (Fisher cat #A117-50) in MilliQ H2O is then added to each well followed by mixing with a pipette. The plate is then resealed with aluminum seal and stored until mass spectrometry analysis.

The extent of covalent modification of KRAS proteins is determined by liquid chromatography electrospray mass spectrometry analysis of the intact proteins on a Thermo Q-Exactive Plus mass spectrometer. 20 μl of sample is injected onto a bioZen 3.6 μm Intact C₄ column (Phenomenex cat #OOB-4767-AN) placed in a column oven set to 40° C. and separated using a suitable LC gradient from ˜20% to ˜60% solvent B. Solvent A is 0.1% formic acid and solvent B is 0.1% formic acid in acetonitrile. HESI source settings are set to 40, 5 and 1 for the sheath, auxiliary and sweep gas flow, respectively. The spray voltage is 4 kV, and the capillary temperature is 320° C. S-lens RF level is 50 and auxiliary gas heater temperature is set to 200° C. The mass spectrometry is acquired using a scan range from 650 to 1750 m/z using positive polarity at a mass resolution of 70,000, AGC target of 1e6 ions and maximum injection time of 250 ms. The recorded protein mass spectrum is deconvoluted from the raw data file using Protein Deconvolution v4.0 (Thermo). The protein mass and adduct masses are exported with their peak intensities. The peak intensities for the unmodified and modified protein are used to calculate the percent covalent modification of the KRAS protein based on the following equation: % KRAS protein modification=((KRAS-compound)/(KRAS)+(KRAS-Compound))*100. One or more exemplified compounds (including compound nos. 201, 203, 211, 225, 231, 237, 241, 243, 244, 246, 255, 256, 257, 261, 262, 266, 267, 270, 273, 275, 278, 281, and 283) exhibited the ability to crosslink Kras mutant G12S and/or G12C greater than 0%, 10%, 20%, 30%, 40%, 50%, 60%, 70%, 80%, 90% or up to 100% within 24 hours when assessed in the assay described above. In embodiments, one or more exemplified compounds herein exhibited the ability to crosslink Kras mutant G12S and G12C greater than 0%, 10%, 20%, 30%, 40%, 50%, 60%, 70%, 80%, 90% or up to 100% within 24 hours when assessed in the assay described above. In embodiments, one or more exemplified compounds herein exhibited the ability to crosslink Kras mutant G12S greater than 0%, 10%, 20%, 30%, 40%, 50%, 60%, 70%, 80%, 90% or up to 100% within 24 hours when assessed in the assay described above. In embodiments, one or more exemplified compounds herein exhibited the ability to crosslink Kras mutant G12C greater than 0%, 10%, 20%, 30%, 40%, 50%, 60%, 70%, 80%, 90% or up to 100% within 24 hours when assessed in the assay described above.

### Example 9: Ras Cellular Assay

The ability of any compound of the present disclosure to inhibit a Ras protein signaling can be demonstrated by inhibiting growth of a given Kras mutant cells. For example, this assay can be also used to assess a selective growth inhibition of a mutant Ras protein relative to a wildtype, or relative to a different mutant Ras protein.

a. Growth of Cells with K-Ras G12C Mutation

MIA PaCa-2 (ATCC CRL-1420) and NCI-H1792 (ATCC CRL-5895) cell lines comprise a G12C mutation and can be used to assess Ras cellular signaling in vitro, e.g., in response to a subject inhibitor compounds of the present disclosure. This cellular assay can also be used to discern selective inhibition of a subject compounds against certain types of Kras mutants, e.g., more potent inhibition against KrasG12D relative to KrasG12C mutant, by using MIA PaCa-2 (G12C driven tumor cell line) as a comparison. MIA PaCa-2 culture medium is prepared with DMEM/Ham's F12 (e.g., with stable Glutamine, 10% FCS, and 2.5% Horse Serum. NCI-H1792 culture medium is prepared with RPMI 1640 (e.g., with stable Glutamine) and 10% FCS.

On a first day (e.g., Day 1), Softagar (Select Agar, Invitrogen, 3% in ddH₂O autoclaved) is boiled and tempered at 48° C. Appropriate culture medium (i.e., medium) is tempered to 37° C. Agar (3%) is diluted 1:5 in medium (=0.6%) and 50 mI/well plated into 96 well plates (Corning, #3904), then incubated at room temperature for agar solidification. A 3% agar is diluted to 0.25% in medium (1:12 dilution) and tempered at 42° C. Cells are trypsinized, counted, and tempered at 37° C. The cells (e.g., MIA PaCa-2 at about 125-150 cells, NCI-H1792 at about 1000 cells) are resuspended in 100 mL 0.25% Agar and plated, followed by incubation at room temperature for agar solidification. The wells are overlaid with 50 mL of the medium. Sister wells in a separate plate are plated for time zero determination. All plates are incubated overnight at 37° C. and 5% CO₂.

On a second day (e.g., Day 2), time zero values are measured. A 40 mL volume of Cell Titer 96 Aqueous Solution (Promega) is added to each well and incubated in the dark at 37° C. and 5% CO₂. Absorption can be measured at 490 nm and reference wavelength 660 nm. DMSO-prediluted test compounds are added to wells of interest, e.g., with HP Dispenser, to one or more desired concentrations (e.g., a final DMSO concentration of 0.3%).

On a tenth day (e.g., Day 10), absorption by wells treated with the test compounds and control wells are measured with, for example, Cell Titer 96 AQueous and analyzed in comparison to the time zero measurements. The IC50 values are determined using the four parameter fit. The resulting IC50 value is a measurement of the ability of the compounds herein to reduce cell growth of Ras-driven cells (e.g., tumor cell lines) in vitro and/or in vivo.

b. Growth of Cells with K-Ras G12D Mutation

ASPC-1 (ATCC CRL-1682), Panc-10.05 (ATCC CRL-2547), A427 cell lines comprise a G12D mutation and can be used to assess Ras cellular signaling in vitro, e.g., in response to the compounds herein. ASPC-1 culture medium is prepared with RPMI-1640 and 10% heat-inactivated FBS. Panc-10.05 culture medium is prepared with RPMI-1640, 10 Units/ml human recombinant insulin, and 10% FBS. A427 cell culture is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384UZ). The day after plating, cells are treated with a dilution series (e.g., a 9 point 3-fold dilution series) of the compounds herein (e.g., approximately 40 μl final volume per well). Cell viability can be monitored (e.g., approximately 5 days later) according to the manufacturer's recommended instructions, where the CellTiter-Glo reagent is added (e.g., approximately 10 μl), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the compounds herein to reduce cell growth of Ras-driven cells (e.g., tumor cell lines) in vitro and/or in vivo. The IC50 values are determined using the four parameter fit. The resulting IC50 value is a measurement of the ability of the compounds herein to reduce cell growth of Ras-driven cells (e.g., tumor cell lines) in vitro and/or in vivo. The ability of one or more compounds exemplified in Table 1 to inhibit growth of one or more cell lines comprising a given Kras mutation is demonstrated utilizing the procedures described above.

c. Growth of Cells with K-Ras G12S Mutation

A549 (ATCC CRL-185) and LS123 (ATCC CRL-255) cell lines comprise a G12S mutation and can be used to assess Ras cellular signaling in vitro, e.g., in response to the compounds herein. A549 culture medium is prepared with RPMI-1640 and 10% heat-inactivated FBS. LS123 culture medium is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384WZ). The day after plating, cells are treated with a dilution series (e.g., a 10 point 3-fold dilution series) of the compounds herein (e.g., approximately 40 μl final volume per well). Cell viability can be monitored (e.g., approximately 6 days later) according to the manufacturer's recommended instructions, where the CellTiter-Glo reagent is added (e.g., approximately 10 μl), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the compounds herein to reduce cell growth of Ras-driven cells (e.g., tumor cell lines) in vitro and/or in vivo. The ability of one or more compounds exemplified in Table 1 to inhibit growth of one or more cell lines comprising a given Kras mutation is demonstrated utilizing the procedures described above. One or more compounds disclosed herein selectively inhibits growth of cells with a K-Ras G12S mutation compared to cells with a K-Ras G12D mutation and/or wildtype K-Ras.

### Example 10: In Vivo Ras Inhibition

The in vivo reduction in Ras signaling output by a compound of the present disclosure is determined in a mouse tumor xenograft model, such as a K-Ras G12D model utilizing cells including a KRas G12D mutant or a K-Ras G12C model utilizing cells including a KRas G12C mutant, or a K-Ras G12S model utilizing cells including a KRas G12S mutant.

Xenograft with K-Ras G12D, G12C, or G12S Mutation

Tumor xenografts are established by administration of tumor cells with a K-Ras G12D mutation (e.g., ASPC-1 cells), a K-Ras G12C mutation (e.g., MIA PaCa-2 cells), or a K-Ras G12S mutation (e.g., A549 or LS123 cells) into mice. Female 6- to 8-week-old athymic BALB/c nude (NCr) nu/nu mice are used for xenografts. The tumor cells (e.g., approximately 5×10⁶) are harvested on the day of use and injected in growth-factor-reduced Matrigel/PBS (e.g., 50% final concentration in 100 μL). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L×w²)/2, in which L and w refer to the length and width of the tumor, respectively. Percent tumor volume change can be calculated using the following formula: (V_{final}−Vᵢₙᵢₜₐₗ)/Vᵢₙᵢₜᵢₐₗ×100. Percent of tumor growth inhibition (% TGI) can be calculated using the following formula: % TGI=100×(1−(average V_{final}−Vᵢₙᵢₜₐₗ of treatment group)/(average V_{final}−Vᵢₙᵢₜᵢₐₗ of control group). When tumors reach a threshold average size (e.g., approximately 200-400 mm³), mice are randomized into 3-10 mice per group and are treated with vehicle (e.g., 100% Labrasol®) or a compound disclosed herein, using, for example, a daily schedule by oral gavage. Results can be expressed as mean and standard deviation of the mean.

### Example 11: Metabolic (Microsomal) Stability Assay

The metabolic stability of a test compound is assayed at 37° C. using pooled liver microsomes (mouse or human liver microsomes). An aliquot of 10 μL of 50 μM test compound is mixed with 490 μL of 0.611 mg/mL liver microsomes, then 50 μL of the mixtures are dispensed to the 96 well tubes and warmed at 37° C. for 10 minutes. The reactions are initiated by adding 50 μL of the pre-warmed NADPH regeneration system solution (add 1.2 μL solution, 240 μl solution B, mix with 10.56 ml KPBS) and then incubated at 37° C. The final incubation solution contains 100 mM potassium phosphate (pH 7.4), 1.3 mM NADP+, 3.3 mM glucose 6-phosphate, 0.4 Unit/mL of glucose 6-phosphate dehydrogenase, 3.3 mM magnesium chloride, 0.3 mg/mL liver microsomes and 0.5 μM test article. After 0, 15, 30 and 60 minutes in a shaking incubator, the reactions are terminated by adding 100 μL of acetonitrile containing 200 nM buspirone as an internal standard. All incubations are conducted in duplicate. Plates are vortexed vigorously by using Fisher Scientific microplate vortex mixer (Henry Troemner, US). Samples are then centrifuged at 3500 rpm for 10 minutes (4° C.) using Sorvall Legend XRT Centrifuge (Thermo Scientific, GE). Supernatants (40 μL) are transferred into clean 96-deep well plates. Each well is added with 160 μL of ultrapure water (Milli-Q, Millipore Corporation) with 0.1% (v/v) formic acid (Fisher Chemical), mixed thoroughly and subjected to LC/MS/MS analysis in MRM positive ionization mode.

All the samples are measured using a mass spectrometer (QTrap 5500 quadrupole/ion trap) coupled with a Shimadzu HPLC system. The HPLC system consisted of a Shimadzu series degasser, binary quaternary gradient pumps, column heater coupled to an autosampler, and a Phenomenex Gemini-NX, C18, 3.0 μm or Phenomenex Lunar, C8, 5.0 μM HPLC column (Phenomenex, Torrance, CA), and eluted with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+).

The half-life for the metabolic degradation of the test compound is calculated by plotting the time-course disappearance of the test compound during the incubation with liver microsomes. Each plot is fitted to a first-order equation for the elimination of the test compound (% remaining compound) versus time using non-linear regression (Equation 1).

$$\frac{{C}_{t}}{{C}_{0}} = {e}^{- kt} \qquad Equation 1$$

where Cₜ is the mean relative substrate concentration at time t and C₀ is the initial concentration (0.5 μM) at time 0. Note that the area ratio of the substrate peak to an internal standard peak is proportional to the analyte concentration and is used for regression analysis to derive a value of k.

The half-life t_{1/2} for metabolic (microsome) stability is derived from the test compound elimination constant k using Equation 2 below.

$${t}_{1 / 2} = \frac{0.693}{k} \qquad Equation 2$$

### Example 12: CYP2C19 Inhibition Assay

Some xenobiotics can inhibit cytochrome P450 (CYP) enzyme function, which alters their ability to metabolize drugs. Administration of a CYP inhibitor with a drug whose clearance is dependent on CYP metabolism can result in increased plasma concentrations of this concomitant drug, leading to potential toxicity. The inhibition of CYP2C19 by a test compound is assayed in human liver microsomes using S-Mephenytoin as a CYP2C19 substrate. The stock solution of the test compound or known CYP2C19 inhibitor as a positive control (10 mM) is diluted with KPBS to 40 μM. In a similar way, the stock solutions of the human liver microsomes and S-Mephenytoin are diluted with KPBS buffer. The pre-incubations are started by incubating a plate containing 25 μL human liver microsomes (final concentration of 0.2 mg/mL), 25 μL NADPH-generating system, and a 25 μL test compound (final concentration 10 μM) or the positive control for 30 min at 37±1° C. After the pre-incubation, 25 μL S-Mephenytoin (final concentration 200 μM) is added and incubated another 12 minutes at 37±1° C. for substrate metabolism. The reactions are terminated by addition of 100 μL of ice-cold acetonitrile containing an internal standard (buspirone). Precipitated proteins are removed by centrifugation at 3500 rpm for 10 minutes at 4° C. (Allegra 25R, Beckman Co. Fullerton, CA) and then aliquot of the supernatant is transferred to an assay plate.

All the samples are assessed using a mass spectrometer (QTrap 5500 quadrupole/ion trap) coupled with a Shimadzu HPLC system, following the manufacturer's instructions. The metabolism of S-Mephenytoin in human liver microsomes is monitored by LC/MS/MS as representative of CYP2C19 inhibitory activity. The amount of metabolite formed is assessed by the peak area ratio (metabolite/IS) and % inhibition at 10 μM is expressed as a percentage of the metabolite signal reduced compared to the control (i.e. an incubation that contained no inhibitor and represented 100% enzyme activity): % inhibition=(1−A/B)×100%, where A is the metabolite peak area ratio formed in the presence of test compound or inhibitor at 10 μM and B is the metabolite peak area ratio formed without test compound or inhibitor in the incubation.

### Example 13: Mouse and Human Protein Binding Assay to Assess Free Drug Concentration

This assay can be used to determine the plasma protein binding of the test compound in the plasma of human and animal species using a Rapid Equilibrium Dialysis (RED) device for equilibrium dialysis and LC-MS/MS for sample analysis. Test compound is spiked in. The stock solution of the test compound is prepared at 5 mM concentration. One μL of 5 mM working solution is added into 1000 μL plasma to achieve a final concentration of 5 μM. The spiked plasma is placed on a rocker, and gently agitated for approximately 20 minutes. A volume of 300 μL of the plasma sample containing 5 μM test compound from each species is added to designate RED device donor chambers followed by addition of 500 μL of potassium phosphate buffer to the corresponding receiver chambers in duplicate. The RED device is then sealed with sealing tape and shaken at 150 RPM for 4 hours at 37° C. Post-dialysis donor and receiver compartment samples are prepared for LC-MS/MS analysis, including spiking samples with an internal standard for the bioanalytical analysis. Warfarin and propranolol are purchased from Sigma-Aldrich (St. Louis, MO), and used as positive controls for low and high plasma protein binding, respectively.

All the samples are analyzed using an Agilent Technologies 6430 Triple Quad LC/MS system. The HPLC system consists of an Agilent 1290 Infinity Liquid Chromatograph coupled to an autosampler (Agilent 1290 Infinity LC Injector HTC), and a Phenomenex Gemini-NX, C18, 3.0 μm or Phenomenex Lunar, C8, 5.0 μM HPLC column (Phenomenex, Torrance, CA), eluting with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+). The percentage of the test compound bound to plasma is calculated following Equation 3 and 4.

$$\% Free test compound = \frac{\begin{matrix}Peak ratio ( \frac{test compound}{Internal standard} ) , \\ receiver compartment\end{matrix}}{\begin{matrix}Peak ratio ( \frac{test compound}{Internal standard} ) , \\ donor compartment\end{matrix}} * 100 \qquad Equation 3$$

$$\% Plasma protein bound test compound = 100 - \% Free test compound \qquad Equation 4$$

### Example 14: hERG (Automated Patch-Clamp) Assay

The human ether-a-go-go related gene (hERG) encodes the voltage gated potassium channel in the heart (IKr) which is involved in cardiac repolarization. Inhibition of the hERG causes QT interval prolongation and can lead to potential fatal events in humans. It is thus important to assess hERG inhibition early in drug discovery. A hERG automated patch-clamp assay is done using a hERG CHO-K1 cell line using an incubation time of 5 min. The degree of hERG inhibition (%) is obtained by measuring the tail current amplitude, which is induced by a one second test pulse to −40 mV after a two second pulse to +20 mV, before and after drug incubation (the difference current is normalized to control and multiplied by 100 to obtain the percent of inhibition). The percent hERG inhibition is measured in the presence of 10 μM test compound.

### Example 15: Rat Oral Exposure (% F)

A pharmacokinetic profile for a test compound is measured by single dosing in jugular vein cannulated male Sprague-Dawley rats. Animal weights are typically over 200 grams, and animals are allowed to acclimate to their new environment for at least 3 days prior to the initiation of any studies. One set of animals is dosed intravenously (IV) with test compound (2 mg/kg in 20% HP-beta-CD or 20% Captisol, pH adjusted to ˜4 by citric acid). The IV dosing solution concentration is 0.4 mg/mL test compound. Blood is sampled at 5 minutes, 15 minutes, 30 minutes, 90 minutes, 360 minutes, and 24 hours following IV dosing. Another set of animals is dosed oral (po) with test compound (10 mg/kg in 20% HP-beta-CD or 20% Captisol, pH adjusted to ˜4 by citric acid). The oral dosing solution concentration is 1 mg/mL test compound. Blood is sampled at 15 minutes, 30 minutes, 90 minutes, 180 minutes, 360 minutes and 24 hours following oral (po) dosing. Blood samples (˜0.2 mL/sample) is collected via the jugular vein, placed in tubes containing EDTA-K2 and stored on ice until centrifuged. The blood samples are centrifuged at approximately 6800 g for 6 minutes at 2-8° C. and the resulting plasma is separated and stored frozen at approximately −80° C.

The plasma samples are analyzed using an Agilent Technologies 6430 Triple Quad LC/MS system, following the manufacturer's instructions. The analytes are detected with positive-mode electrospray ionization (ES+). A standard curve for each test compound is generated and used to measure test compound concentrations in the rat plasma samples. Based on the time course sampling, an area under the curve is calculated for the oral dose group and the intravenous dose group. Percentage rat bioavailability is calculated based on equation 5.

$$\% F ( rat ) = \frac{{AUC}_{po} * {Dose}_{IV}}{{AUC}_{IV} * {Dose}_{po}} , \qquad Equation 5$$

where F is bioavailability, AUCₚₒ is area under curve of oral drug, AUC_{IV} is area under curve of intravenous drug, Dose_{IV} is the intravenous dose and Doseₚₒ is the oral dose.

Besides the cellular proliferation inhibitory effect and high potency in reducing K-Ras signaling, particularly signaling mediated by K-Ras mutant, compounds disclosed herein exhibit advantageous ADME and/or DMPK properties. Fine-tuned pharmacological properties are of great significance for improving efficacy and safety of K-Ras inhibitors for therapeutic clinical applications.

In some embodiments, a compound of the present disclosure exhibits at least one, two, three or more advantageous pharmacological properties. Exemplary superior DMPK properties may include but are not limited to improved metabolic stability, reduced hERG liability, decreased CYP inhibition, increased oral exposure, and decreased serum protein binding (hence increasing the amount of free and available compound circulating in a subject's blood following administration of the compound).

In some embodiments, a compound of the present disclosure exhibits suitable microsomal stability.

In some embodiments, a subject compound exhibits suitable metabolic stability as ascertained by a T½ of mouse liver microsomal metabolism greater than 10 mins, 20 mins, 30 mins, 40 mins, 50 mins, 60 mins or longer as (see Example 11 for experimental procedures). In some embodiments, a subject compound exhibits suitable metabolic stability as ascertained by a T½ of human liver microsomal metabolism greater than 10 mins, 20 mins, 30 mins, 40 mins, 50 mins, 60 mins, 100 mins, 120 mins or longer as (see Example 11 for experimental procedures). One or more compounds disclosed herein are expected to exhibit a suitable microsomal stability with a T½ greater than 10 mins, 20 mins, 30 mins, 40 mins, 50 mins, 60 mins or longer in mouse and/or human liver microsomal metabolism assays.

1. A compound of Formula (I), or a pharmaceutically acceptable salt or solvate thereof: 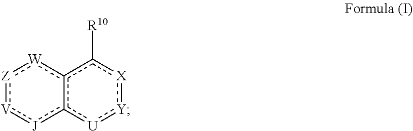
wherein
W is a N, C(R¹⁸), N(R^{18b}), C(R¹⁸)(R^{18a}), C(O), S(O), or S(O)₂;
Z is N, C(R⁸), N(R^{8b}), C(R⁸)(R^{8a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}) and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), or S(O)₂;
U is N, C(R^{2c}), C(R^{2c})(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is 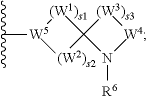
each W¹ and each W³ are independently selected from N(R¹), N(R⁴), C(R¹)(R¹), C(R¹)(R⁴), C(R⁴)(R⁴), C(O), S, O, S(O), and S(O)₂;
each W² is independently selected from N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), S(O), and S(O)₂;
W⁵ is selected from N, C(R¹), and C(R⁴);
s1 is 1 and s2 is 0, 2, or 3; s1 is 2 and s2 is 0, 1, or 3; s1 is 3 and s2 is 0 to 3; s1 is 4 and s2 is 0 to 3;
s1 is 5 and s2 is 0 to 3; or s1 is 6 and s2 is 0 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₆heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵ and wherein R⁶ is optionally capable of forming a covalent bond with a Ras amino acid;
L² is a bond, —O—, —N(R^{4d})—, —C(O)—, —S—, —S(O)₂—, —S(O)—, —P(O)R^{4d}—, CR^{4c}R^{4c}, —OCR^{4c}R^{4d}—, —N(R^{4d})CR^{4c}R^{4c}—, —C(O)CR^{4c}R^{4c}—, —SCR^{4c}R^{4c}—, —S(O)₂CR^{4c}R^{4c}—, —S(O)CR^{4c}R^{4c}—, —P(O)R^{4d}CR^{4c}R^{4c}, —CR^{4c}R^{4c}CR^{4c}R^{4c}, —CR^{4c}R^{4c}O—, —CR^{4c}R^{4c}N(R^{4d})—, —CR^{4c}R^{4c}C(O)—, —CR^{4c}R^{4c}S—, —CR^{4c}R^{4c}S(O)₂—, —CR^{4c}R^{4c}S(O)—, —CR^{4c}R^{4c}, —P(O)R^{4d}—, —N(R^{4d})C(O)—, —N(R^{4d})S(O)₂—, —N(R^{4d})S(O)—, —N(R^{4d})P(O)R^{4d}—, —C(O)N(R^{4d})—, —S(O)₂N(R^{4d})—, —S(O)N(R^{4d})—, —P(O)R^{4d}N(R^{4d})—, —OC(O)—, —OS(O)₂—, —OS(O)—, —OP(O)R^{4d}—, —C(O)O—, —S(O)₂O—, —S(O)O—, —P(O)R^{4d}O—, —CR^{4c}R^{4c}CR^{4c}R^{4c}CR^{4c}R^{4c}—, —OCR^{4c}R^{4c}CR^{4c}R^{4c}—, —N(R^{4d})CR^{4c}R^{4c}CR^{4c}R^{4c}—, —C(O)CR^{4c}R^{4c}CR^{4c}R^{4c}—, —SCR^{4c}R^{4c}CR^{4c}R^{4c}—, —S(O)₂CR^{4c}R^{4c}CR^{4c}R^{4c}—, —S(O)CR^{4c}R^{4c}CR^{4c}R^{4c}—, —P(O)R^{4d}CR^{4c}R^{4c}CR^{4c}R^{4c}—, —CR^{4c}R^{4c}CR^{4c}R^{4c}O—, —CR^{4c}R^{4c}CR^{4c}R^{4c}N(R^{4d})—, —CR^{4c}R^{4c}CR^{4c}R^{4c}C(O)—, —CR^{4c}R^{4c}CR^{4c}R^{4c}S—, —CR^{4c}R^{4c}CR^{4c}R^{4c}S(O)₂—, —CR^{4c}R^{4c}CR^{4c}R^{4c}S(O)—, or —CR^{4c}R^{4c}CR^{4c}R^{4c}P(O)R^{4d}—;
each R^{4c} is independently selected from hydrogen, halogen, —CN, C₁₋₆alkyl, C₂₋₆alkenyl, C₂₋₆alkynyl, C₁₋₆haloalkyl, C₁₋₆alkoxy, C₁₋₆haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, —OC(O)R^{14a}, —N(R¹⁴)(R¹⁴), —N(R¹⁴)C(O)N(R¹⁴)(R¹⁴), —N(R¹⁴)C(O)OR¹⁴, —N(R¹⁴)C(O)R^{14a}, and —N(R¹⁴)S(O)₂R¹⁴, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
each R^{4d} is independently selected from hydrogen, —CN, C₁₋₆alkyl, C₂₋₆alkenyl, C₂₋₆alkynyl, C₁₋₆haloalkyl, C₁₋₆alkoxy, C₁₋₆haloalkoxy, C₃₋₁₀cycloalkyl, —CH₂—C₃₋₁₀cycloalkyl, C₂₋₉heterocycloalkyl, —CH₂—C₂₋₉heterocycloalkyl, —OR¹⁴, —SR¹⁴, —C(O)OR¹⁴, —C(O)N(R¹⁴)(R¹⁴), —C(O)C(O)N(R¹⁴)(R¹⁴), —OC(O)N(R¹⁴)(R¹⁴), —C(O)R^{14a}, —S(O)₂R¹⁴, —S(O)₂N(R¹⁴)(R¹⁴), —OCH₂C(O)OR¹⁴, and —OC(O)R^{14a}, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, and —CH₂—C₂₋₉heterocycloalkyl, are optionally substituted with one, two, or three groups independently selected from halogen, oxo, —CN, C₁₋₆ alkyl, C₁₋₆haloalkyl, C₁₋₆alkoxy, C₁₋₆haloalkoxy, —OR¹⁴, —SR¹⁴, —N(R¹⁴)(R¹⁴), —C(O)OR¹⁴, —C(O)N(R¹⁴)(R¹⁴), —C(O)C(O)N(R¹⁴)(R¹⁴), —OC(O)N(R¹⁴)(R¹⁴), —N(R¹⁴)C(O)N(R¹⁴)(R¹⁴), —N(R¹⁴)C(O)OR¹⁴, —N(R¹⁴)C(O)R¹⁴, —N(R¹⁴)S(O)₂R¹⁴, —C(O)R^{14a}, —S(O)₂R¹⁴, —S(O)₂N(R¹⁴)(R¹⁴), and —OC(O)R^{14a};
R⁵ is selected from halogen, —CN, C₁₋₆alkyl, C₂₋₆alkenyl, C₂₋₆alkynyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, C₁₋₁₁heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein the C₁₋₆alkyl, C₂₋₆alkenyl, C₂₋₆alkynyl, C₃₋₁₂cycloalkyl, —CH₂—C₃₋₁₂cycloalkyl, C₁₋₁₁-heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R¹⁷ is -L¹-R¹⁹;
R^{17b} is -L^{1b}-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from a C₃₋₁₂cycloalkyl, C₂₋₁₁heterocycloalkyl, C₆₋₁₂aryl, and C₂₋₁₂heteroaryl, wherein the C₃₋₁₂cycloalkyl, C₂₋₁₁heterocycloalkyl, C₆₋₁₂aryl, and C₂₋₁₂heteroaryl are optionally substituted with one, two, three, four, five, six, or seven R¹ⁱ;
each R¹ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12″})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³), —S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2c} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}—(C₂₋₆alkenyl)-R^{2b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20a};
each R^{12′} is independently selected from C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂, —C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R^{20c}, R^{20d}, R^{20e}, R^{20f}, R^{20g}, R^{20h}, R²⁰ⁱ, R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵;
wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and [symbol]
indicates a single or double bond such that all valences are satisfied.

2. A compound of Formula (II), or a pharmaceutically acceptable salt or solvate thereof: 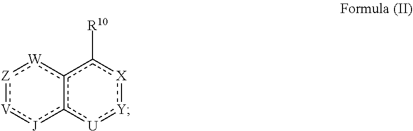
wherein
W is a N, C(R¹⁸), N(R^{18b}), C(R¹⁸)(R^{18a}), C(O), S(O), or S(O)₂;
Z is N, C(R), N(R^{b}), C(R)(R^{a}), C(O), S(O), or S(O)₂; wherein W and Z are not both selected from C(O), S(O), and S(O)₂;
V and J are each independently selected from C(R¹⁷), C(R¹⁷)(R^{16a}), C(R¹⁶), C(R¹⁶)(R^{16a}), N, N(R^{17b}) and N(R^{16b}); wherein exactly one of V and J is C(R¹⁷), C(R¹⁷)(R^{16a}), or N(R^{17b});
Y is N, C(R²), C(R²)(R^{2c}), N(R^{2b}), S(O), S(O)₂, or C(O);
U is N, C(R^{2c}), C(R^{2c})(R^{2c}), N(R^{2b}), S(O), S(O)₂, or C(O);
R¹⁰ is -L⁷-R⁷;
L⁷ is a bond, —O—, —N(R¹⁴)—, —C(O)—, —S—, —S(O)₂—, —S(O)—, C₁₋₄alkyl, or 2-4 membered heteroalkyl linker, wherein the C₁₋₄alkyl and 2-4 membered heteroalkyl linker are each optionally substituted with one, two or three R^{20a};
R⁷ is 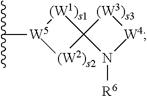
W¹ and W³ are independently selected from N(R¹), N(R⁴), C(R¹)(R¹), C(R¹)(R⁴), C(R⁴)(R⁴), C(O), S, O, S(O), and S(O)₂;
W² is independently selected from a bond, N(R¹), N(R⁴), C(R¹)(R¹), C(R¹)(R⁴), C(R⁴)(R⁴), C(O), S, O, S(O), and S(O)₂;
W⁴ is selected from C(R¹)(R¹), C(R¹)(R⁴), C(R⁴)(R⁴), C(O), S(O), and S(O)₂;
W⁵ is selected from N, C(R¹), and C(R⁴);
s1 is an integer from 1 to 6;
s2 is an integer from 1 to 3;
s3 is an integer from 1 to 3;
each R¹ is independently selected from hydrogen, C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl, wherein C₁₋₆alkyl, C₂₋₆alkenyl, C₂₋₆alkynyl, C₁₋₆haloalkyl, C₃₋₁₂cycloalkyl, —CH₂—C₃₋₁₂cycloalkyl, C₁₋₁₁heterocycloalkyl, —CH₂—C₁₋₁₁heterocycloalkyl, C₆₋₁₂aryl, —CH₂—C₆₋₁₂aryl, —CH₂—C₁₋₁₁heteroaryl, and C₁₋₁₁heteroaryl are optionally substituted with one, two, or three R^{20a};
each R^{4c} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), ˜CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R⁶ is -L²-R⁵ and wherein R⁶ is optionally capable of forming a covalent bond with a Ras amino acid;
L² is —C(O)—;
R⁵ is selected from C₃₋₁₂cycloalkyl, C₁₋₁₁heterocycloalkyl, C₆₋₁₂aryl, and C₁₋₁₁heteroaryl, wherein the C₃₋₁₂cycloalkyl, C₁₋₁₁heterocycloalkyl, C₆₋₁₂aryl, and C₁₋₁₁heteroaryl are optionally substituted with one, two, or three R^{20k};
R⁸ and R^{8a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R^{8b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R¹⁷ is -L¹-R¹⁹;
R^{17b} is -L b-R¹⁹;
L¹ is selected from a bond, C₁-C₄alkyl, C₂-C₄alkenyl, C₂-C₄alkynyl, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, —OCON(R¹⁴)—, —N(R¹⁴)C(O)O—, N(R^{1c}), C(O)N(R^{1c}), S(O)₂N(R^{1c}), S(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
L^{1b} is selected from a bond, C₁-C₄alkyl, C₂-C₄alkenyl, C₂-C₄alkynyl, —C(O)—, —C(O)N(R¹⁴)—, C(O)N(R^{1c}), C(R^{1f})(R^{1g})O, C(R^{1f})(R^{1g})N(R^{1c}), and C(R^{1f})(R^{1g}); wherein the C₁-C₄alkyl, C₂-C₄alkenyl, and C₂-C₄alkynyl are optionally substituted with one, two, or three R²⁰ⁱ;
R^{1e}, R^{1f}, and R^{1g} are independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1f} and R^{1g} are joined to form a 4-7 membered heterocycloalkyl ring or a 4-7 membered cycloalkyl ring, wherein the 4-7 membered heterocycloalkyl ring or 4-7 membered cycloalkyl ring are optionally substituted with one, two, or three R²⁰ⁱ;
R^{1c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁹ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, three, four, five, six, or seven R²⁰ⁱ;
each R²⁰ⁱ is independently selected from halogen, oxo, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ;
R¹⁶ and R^{16a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20g};
R^{16b} is independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R² is halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR^{12′}, —SR^{12′}, —N(R^{12′})(R¹³), —C(O)OR^{12′}, —OC(O)N(R^{12′})(R¹³), —N(R¹⁴)C(O)N(R^{12′})(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R^{12′})(R¹³), —C(O)C(O)N(R^{12′})(R¹³), —N(R¹⁴)C(O)R¹⁵, —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2c} is independently hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{2b} is independently hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R^{12′})(R¹³), —C(O)R¹⁵, —S(O)R¹⁵, —OC(O)R¹⁵, —C(O)N(R^{12′})(R¹³), —C(O)C(O)N(R^{12′})(R¹³), —S(O)₂R¹⁵, —S(O)₂N(R^{12′})(R¹³)—, S(═O)(═NH)N(R^{12′})(R¹³), —CH₂C(O)N(R^{12′})(R¹³), —CH₂N(R¹⁴)C(O)R¹⁵, —CH₂S(O)₂R¹⁵, —CH₂S(O)₂N(R^{12′})(R¹³), —(C₁-C₆alkyl)-R^{12b}, —(C₂₋₆alkenyl)-R^{12b}, —(C₂₋₆alkynyl)-R^{12b}, —(C₃₋₁₀cycloalkyl)-R^{12b}, —(C₂₋₉heterocycloalkyl)-R^{12b}, —(C₆₋₁₀aryl)-R^{12b}, or —(C₁₋₉heteroaryl)-R^{12b}, wherein said C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R^{12b} is selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
X is C(R³), C(R³)(R³), N(R³), or N;
each R³ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R^{12′} is independently selected from hydrogen, C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl, wherein C₁₋₆alkyl, C₂₋₆alkenyl, C₂₋₆alkynyl, C₃₋₁₀cycloalkyl, —C(R^{12c})₂—C₃₋₁₀cycloalkyl, C₂₋₉heterocycloalkyl, —C(R^{12c})₂—C₂₋₉heterocycloalkyl, C₆₋₁₀aryl, —C(R^{12c})₂—C₆₋₁₀aryl, —C(R^{12c})₂—C₁₋₉heteroaryl, and C₁₋₉heteroaryl are optionally substituted with one, two, or three R^{20d};
each R^{12c} is independently selected from hydrogen and R^{20m};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R^{14a} is independently selected from C₁₋₆alkyl and C₁₋₆haloalkyl;
each R¹⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
R¹⁸ and R^{18a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
R^{18b} is selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20h};
each R^{20a}, R^{20b}, R^{20c}, R^{20d}, R^{20e}, R^{20f}, R^{20g}, R^{20h}, R²⁰ⁱ, R^{20k}, and R^{20m} is independently selected from halogen, oxo, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵;
wherein two R^{20k} bonded to the same or adjacent atoms may optionally be joined to form a C₃₋₁₀cycloalkyl, C₂₋₉heterocycloalkyl, C₆₋₁₀aryl, or C₁₋₉heteroaryl; wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; and [symbol]
indicates a single or double bond such that all valences are satisfied.

3. The compound of claim 1, or a pharmaceutically acceptable salt or solvate thereof, wherein L² is —C(O)—.

4. The compound of claim 1, or a pharmaceutically acceptable salt or solvate thereof, wherein R⁵ is a 5 or 6 membered partially unsaturated heterocycloalkyl or a 5 or 6 membered heteroaryl, each optionally substituted with one, two or three R^{20k}, wherein the partially unsaturated 5 or 6 membered heterocycloalkyl or 5 or 6 membered heteroaryl comprises one, two, or three ring nitrogen atoms; and each is bonded to L² through a ring nitrogen.

5. The compound claim 1, or a pharmaceutically acceptable salt or solvate thereof, wherein R⁵ is a 5 membered heteroaryl optionally substituted with one, two or three R^{20k}, wherein the 5 membered heteroaryl comprises one, two, or three ring nitrogen atoms; and the 5 membered heteroaryl is bonded to L² through a ring nitrogen.

6. The compound of claim 1, or a pharmaceutically acceptable salt or solvate thereof, wherein L² is —C(O)—; and R⁵ is a C₃₋₁₂cycloalkyl optionally substituted with one, two or three R^{20k}.

7. (canceled)

8. The compound of claim 1, or a pharmaceutically acceptable salt or solvate thereof, wherein L² is —C(O)—; and R⁵ is C₂₋₆alkenyl, wherein C₂₋₆alkenyl is optionally substituted with one, two, or three R^{20k}.

9. The compound of claim 1, or a pharmaceutically acceptable salt or solvate thereof, wherein
L² is —C(O)—; and R⁵ is C₂₋₆alkynyl, wherein C₂₋₆alkynyl is optionally substituted with one, two, or three R^{20k}.

10.-18. (canceled)

19. The compound of claim 1, or a pharmaceutically acceptable salt or solvate thereof, wherein Z is N: Y is C(R²); W is a CH; U is N; J is C(R¹⁶); and X is N.

20. The compound of claim 1, or a pharmaceutically acceptable salt or solvate thereof, wherein Z is C(R⁸); Y is C(R²); W is a CH: U is N; J is C(R¹⁶); and X is N.

21. (canceled)

22. The compound of claim 1, or a pharmaceutically acceptable salt or solvate thereof, wherein Z is N(R^{8b}); Y is C(R²); W is a C(O); U is N; J is C(R¹⁶) and X is N.

23.-45. (canceled)

46. The compound of claim 1, or a pharmaceutically acceptable salt or solvate thereof, wherein R⁷ is selected from 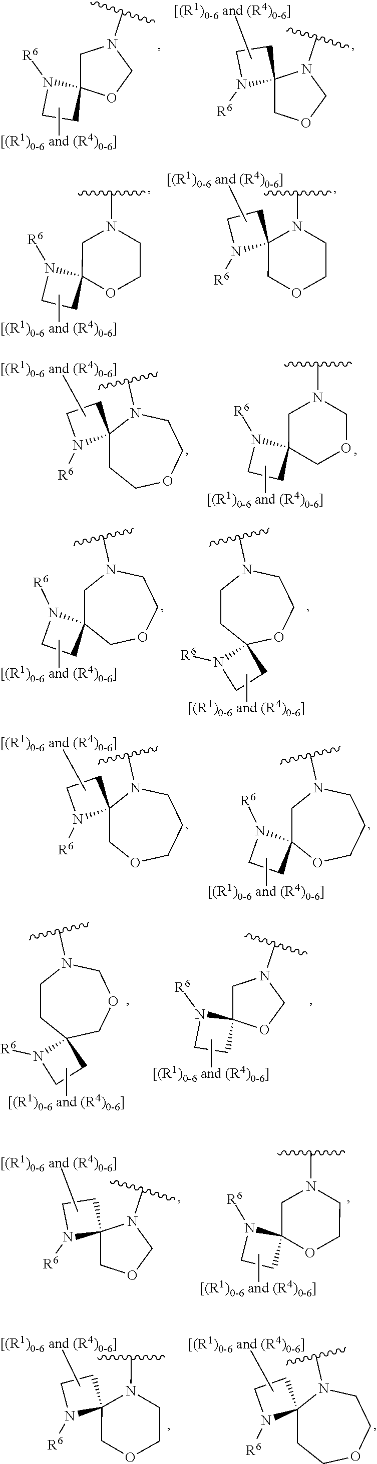 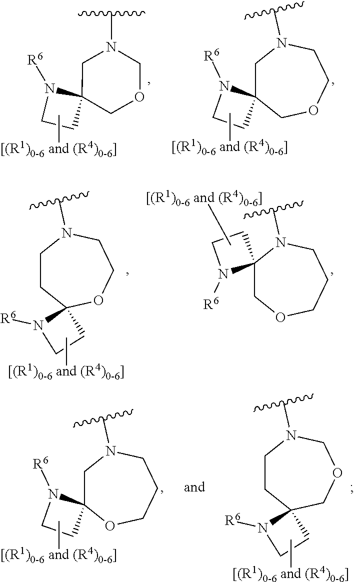
wherein R¹ and R⁴ substituents may be bonded to either spirocyclic ring.

47. The compound of claim 1, or a pharmaceutically acceptable salt or solvate thereof, wherein R⁷ is selected from 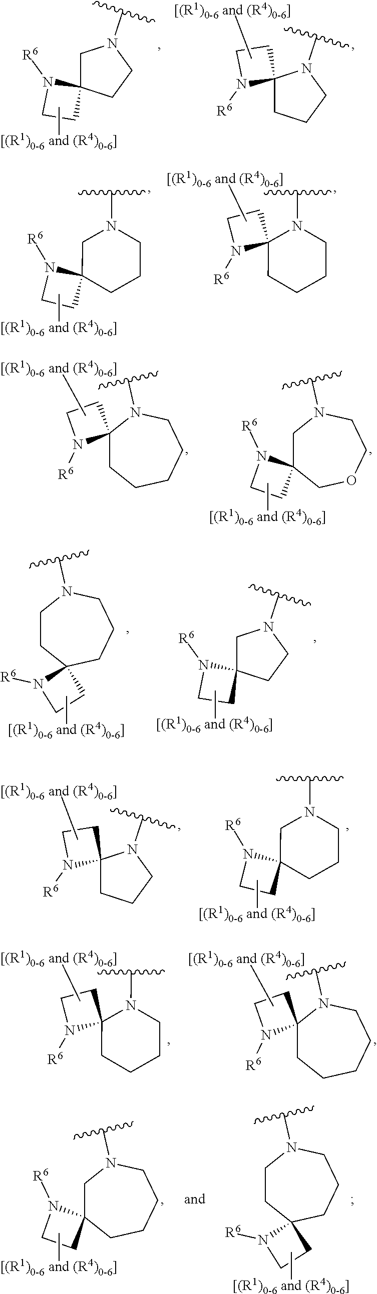
wherein R¹ and R⁴ substituents may be bonded to either spirocyclic ring.

48. The compound of claim 1, or a pharmaceutically acceptable salt or solvate thereof, wherein R⁷ is selected from 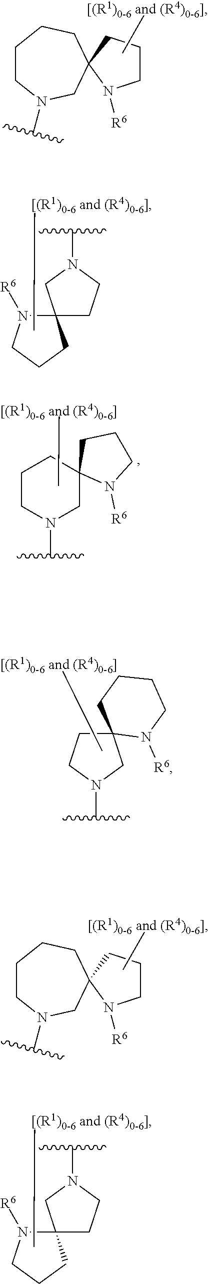 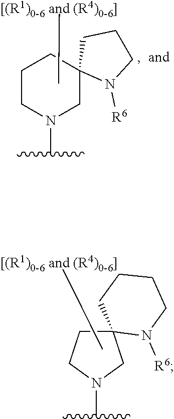
wherein R¹ and R⁴ substituents may be bonded to either spirocyclic ring.

49. (canceled)

50. The compound of claim 1, or a pharmaceutically acceptable salt or solvate thereof, wherein R¹⁷ is -L¹-R¹⁹ or -L^{1b}-R¹⁹; L¹ is a bond, and L^{1b} is a bond.

51. The compound of claim 1, or a pharmaceutically acceptable salt or solvate thereof, wherein R¹⁹ is: 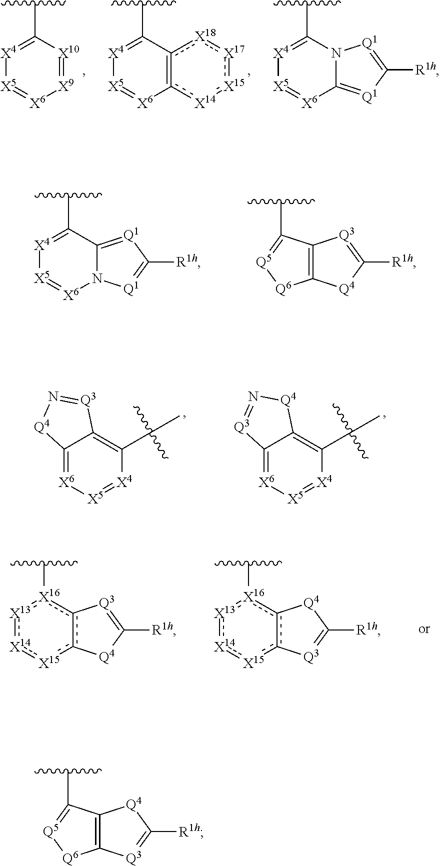
Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}) C(R^{1a})(R^{1b})N(R^{1c}), and N(R^{1c});
X¹⁴, X¹⁵, X¹⁷, X¹⁸ are independently selected from C(O), C(R^{1a}), N, C(R^{1a})(R^{1b}), and N(R^{1c});
X¹⁶ is selected from C, N, and C(R^{1a});
each R^{1a}, R^{1b}, R^{1d}, and R^{1b} is independently selected from hydrogen, halogen, —CN, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ; or R^{1a} and R^{1b} bonded to the same carbon are joined to form a 3-10 membered heterocycloalkyl ring or a C₃₋₁₀cycloalkyl ring, wherein the 3-10 membered heterocycloalkyl ring or C₃₋₁₀cycloalkyl ring are optionally substituted with one, two, or three R²⁰ⁱ; or two R^{1a} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, C₆₋₁₀aryl ring, 5-12 membered heteroaryl ring, or C₃₋₁₀cycloalkyl ring are optionally substituted with one, two, or three R²⁰ⁱ; or R^{1b} and one of R^{1a}, R^{1b}, R^{1c}, and R^{1d} bonded to adjacent atoms are joined to form a 3-10 membered heterocycloalkyl ring, a C₆₋₁₀aryl ring, a 5-12 membered heteroaryl ring, or a C₃₋₁₀cycloalkyl ring, wherein the 3-10 membered heterocycloalkyl ring, a C₆₋₁₀aryl ring, a 5-12 membered heteroaryl ring, and C₃₋₁₀cycloalkyl ring are optionally substituted with one, two, or three R²⁰ⁱ; and
each R^{1c} is independently selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ.

52. The compound of claim 1, or a pharmaceutically acceptable salt or solvate thereof, wherein R¹⁹ is selected from

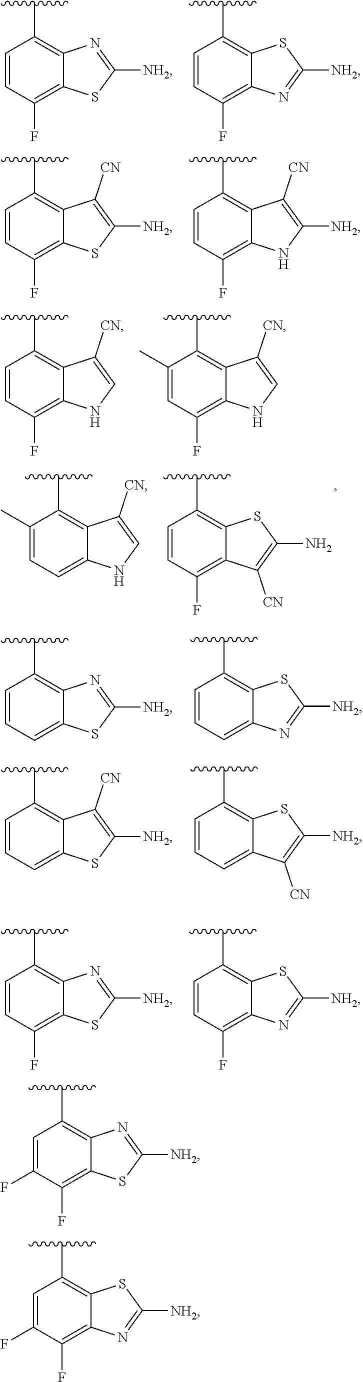

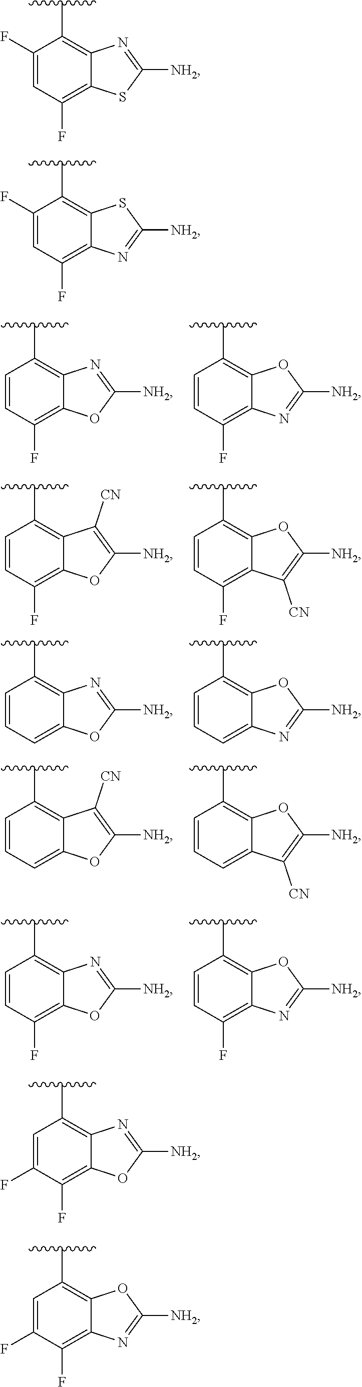

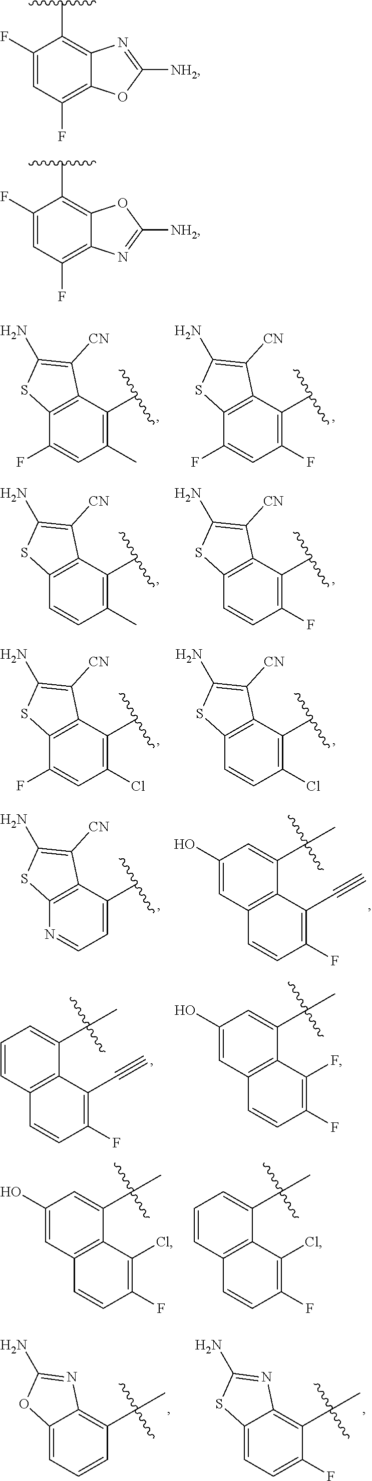

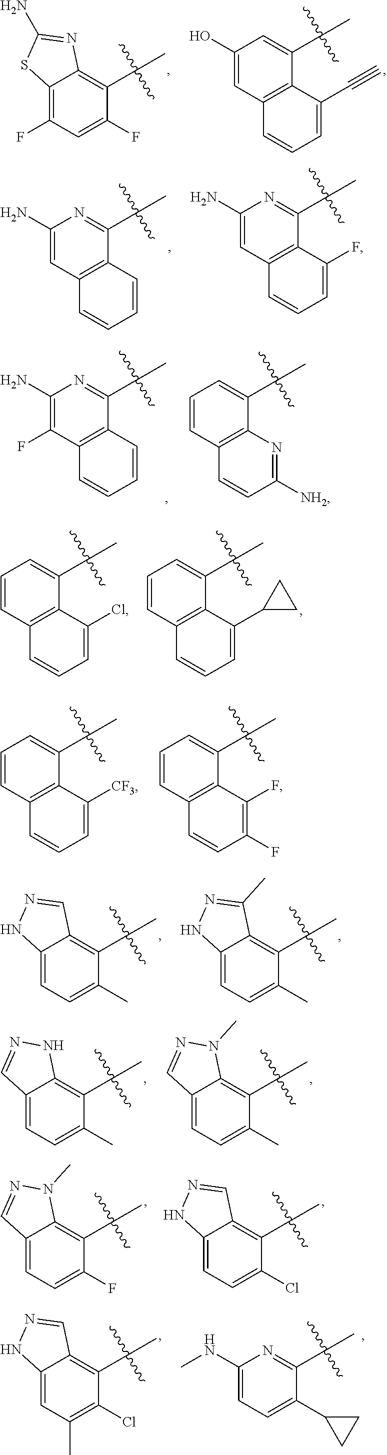

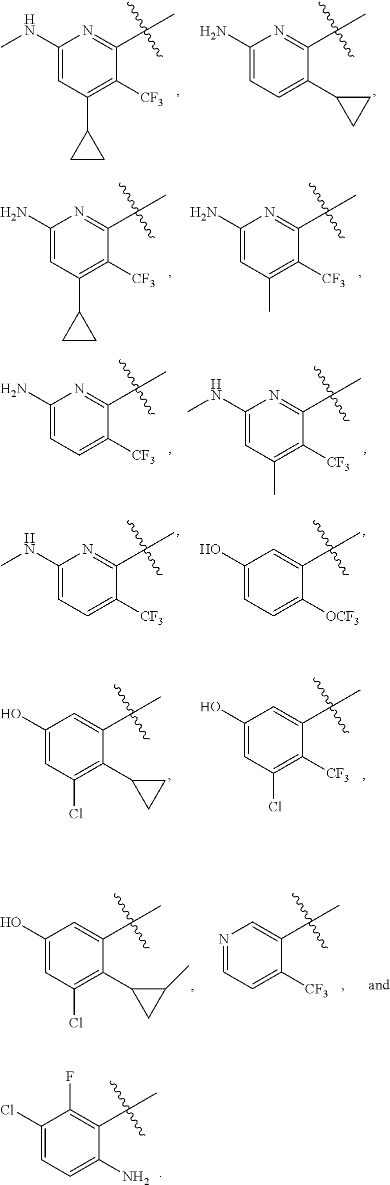

53. (canceled)

54. (canceled)

55. The compound of claim 1, or a pharmaceutically acceptable salt or solvate thereof, wherein R² is selected from

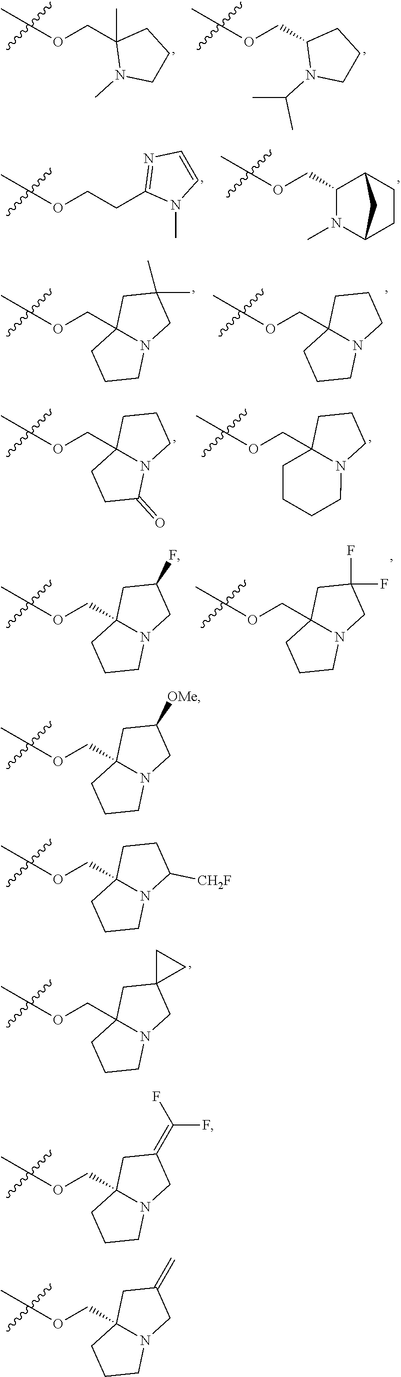

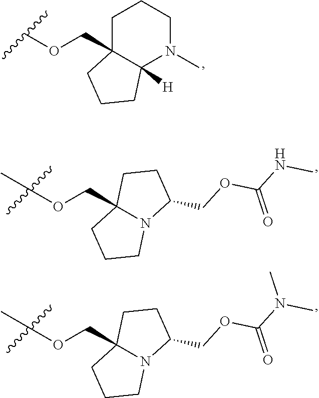 [SMILES: *OC[C@]12CCC[C@H]1N(C)CCC2 ; *OC[C@]12CCCN1[C@@H](COC(=O)NC)CC2 ; *OC[C@]12CCCN1[C@@H](COC(=O)N(C)C)CC2]

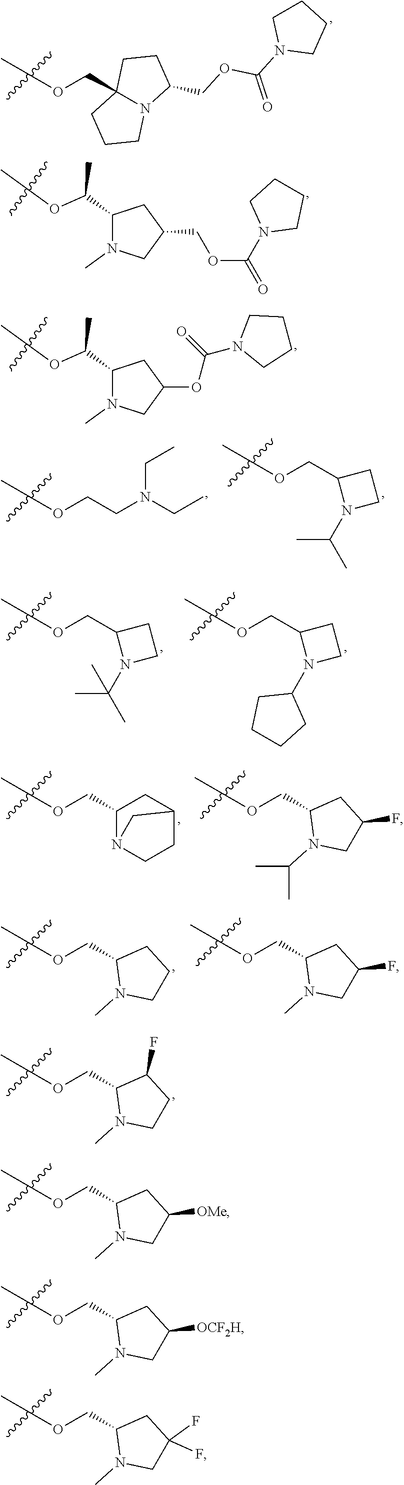

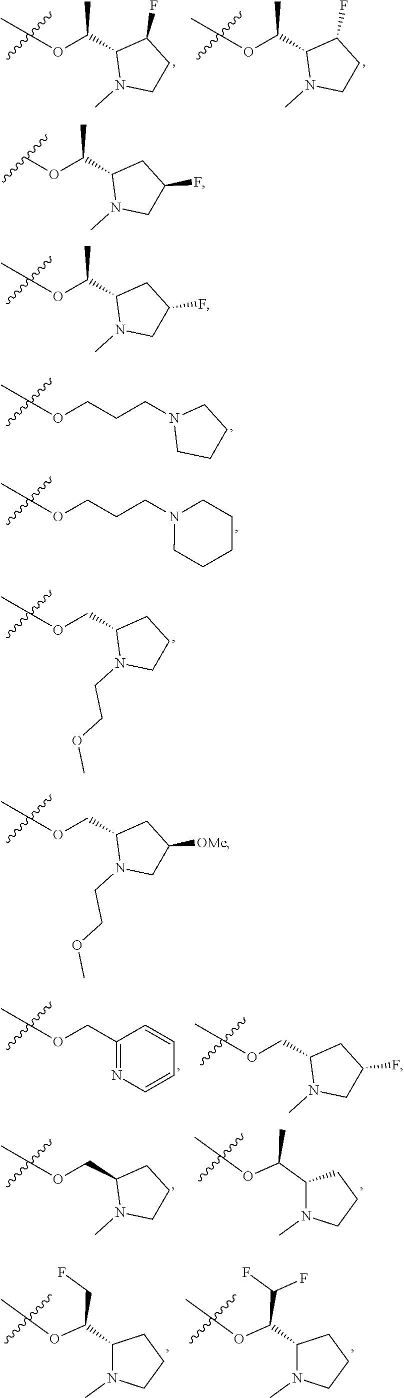

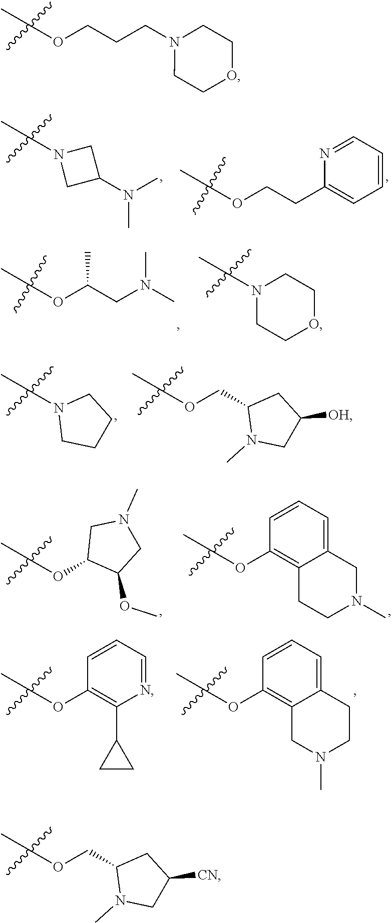

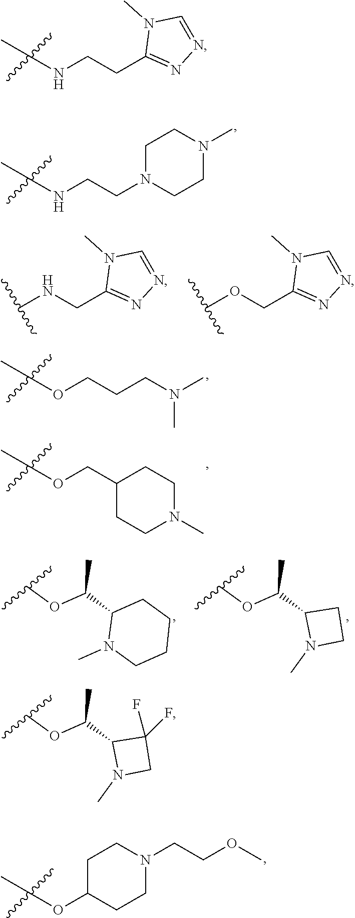

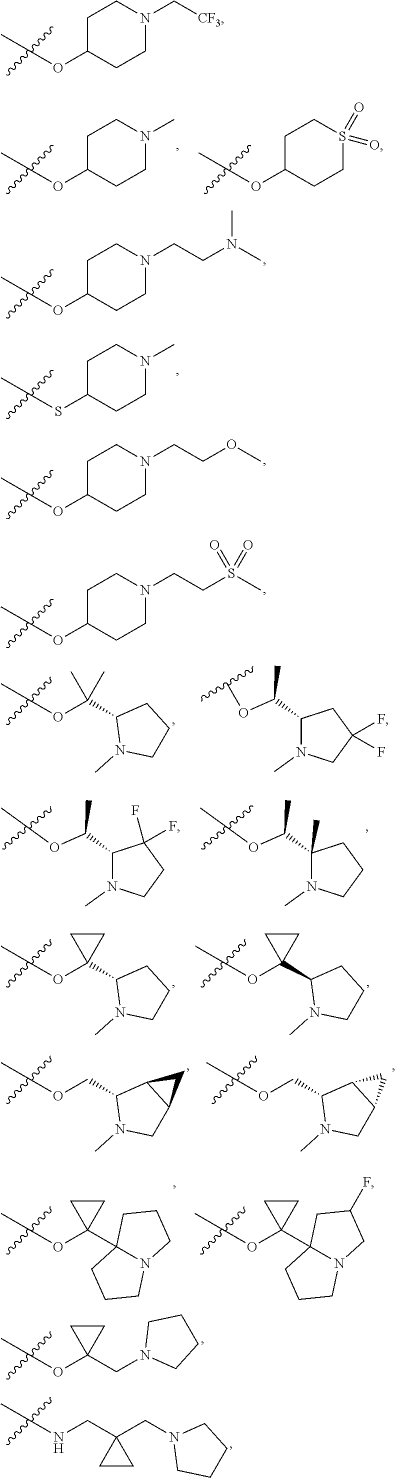

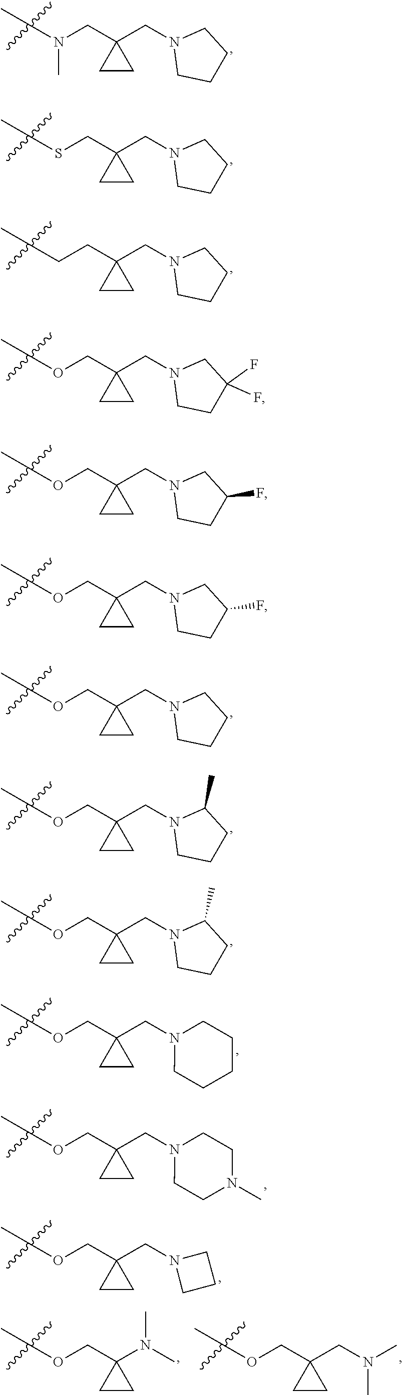

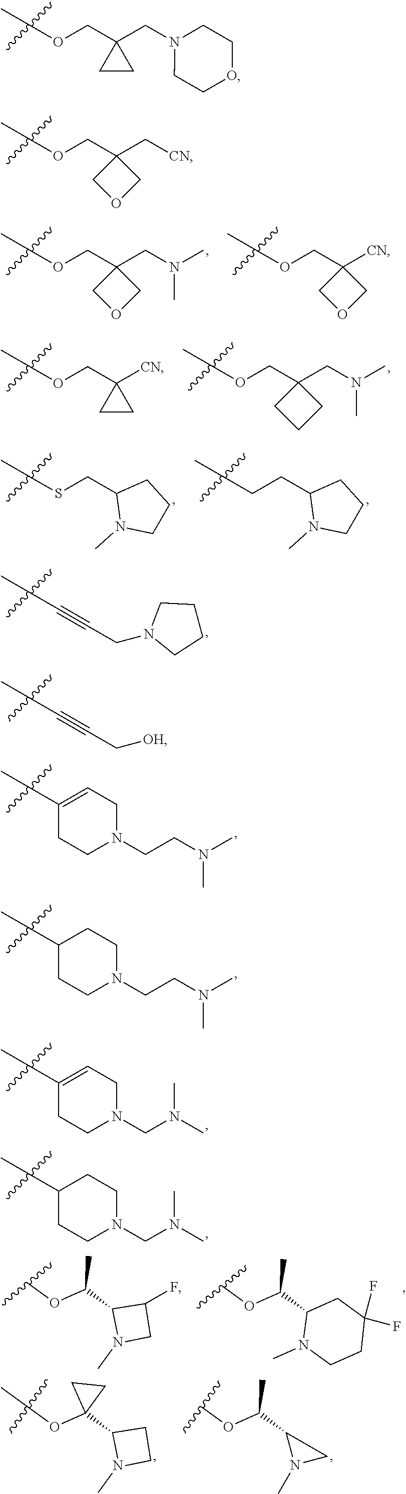

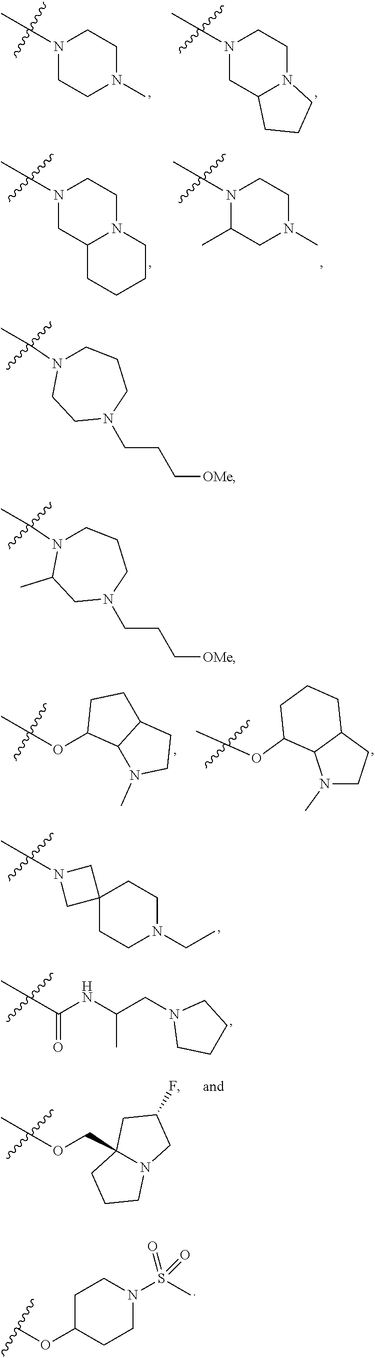

56. (canceled)

57. A pharmaceutical composition comprising a compound of claim 1, or a pharmaceutically acceptable salt or solvate thereof, and a pharmaceutically acceptable excipient.

58. A method of treating cancer in a subject in need thereof, comprising administering to the subject a therapeutically effective amount of a compound of claim 1, or a pharmaceutically acceptable salt or solvate thereof.

59.-62. (canceled)
